SEQUENCE LISTING (526 sequences)

SEQ ID NO: 1 - protein, 1359 aa, synthetic construct
MRETGKDYYLGLDMGTNSVGWAVTDENYVLMRAKGKDLWGIREFDEALTAVDRRTHRVARRRHQREIARIGLLKEYFHDAIAEVDPDFYQRLDNSKYHEEDKDSAVKGKNGIFNDANYNDKDYFKQYPTIFHLRKELIESTEKHDVRLVYLALLNMFKHRGHFLNAGLSTESENTMDTAYHNFVETAAQTIECNFMETVDIEKIKEILGSRDYSRSKKAELVAQILHVDSKNKVQMACIKCICGLKVTAAAIFGDKMAADEEKKTDICFSDFGYDEKVPVILEIVGEENFELVLAMQEIYDIGSLAGIMKDSLYLSMARVKEYEQHGKDLRILKGVVKKYGTKEEYDTLFRTMEEGTYSAYVNSVNTKKKSRRDVKKRTRDEFYGRLKKIISKMPQNDSDVLYIKDKMEKEAFLPKQLTASNGVIPNQIHAREMKKILQNAEIYLPFLKEKDTSGLTVSERILKLFSFQIPYYVGPVSEKSKLNGGNGWVVRKEEGTVLPWNIEEKIDMTATSREFVERMVRRCSYLSDERVLPKGSLLYERFCVLNEINAIKVDGVKIPVEIKQDLYSKYFKKGKKVTKKKIAEFLIARGVIKEDQTELISGIDIAVNSTLSTYAKFYAIFGEKMEQDSYKKMVEQIVEWCTIYGDAKKFLEQKIKECYGNVLTQEEIKRITGFKFRDWGRLSKEFLELSGCSKATGEVMPLISMMWETNDNLMELLDDNYYTYREALEEKQNNAFKLLADIEPEDLDEMYFSAPVKRMVWQTLCIIKELEKVLGKAPKRLFVEMTRNEQKDKKRTDSRKQQFLDLYKNIKDESRDWKELITRADENGTLKSKKMYLYLIQMGRCMYTGHPIDLEDLFKDNRYDIDHIYPRHFVKDDNIMNNLVLVEKEKNAHKSDNYPLEEEIYKKQLPMWKDLHQRKLITDEKFKRLTGRNEFTDEQKAGFIARQLVETSQGTKGVTTILERALPDTKIVYAKASNVSEFRQKRDLLKSRVVNDFHHAQDAYLNIVVGNVYFVKFTQNPLHFIKAEYDKNKAQYEYNLSRMFDRDVRRGNETAWIGCKKGEEAGTILTVKKMMAKNTPMLTRLSFEGKGGLADQTLYSAEKAKGNGYIPLKTSDSKMQDVTKYGGFNSVSTAYFFLVEHELKGKKVRTLETVPIIWRDRIQKDETMLLKYCTDVLELKNPSIRVKKIKLQSYIKKDGYYMYITGKTNKQITTKNAVELCVNQRWIDYIKIIEKTVEKKKEECEEIDYDKNVEFYDLLLTKHTQEIFSNRPNPVGAKLKQGRDKFVSLTLLEQCRVLYEILQLSIIGNTRADLQLIGGAAISGVMLLSKNITDSKEFILINQSVTGLFEQKVDLLTV

SEQ ID NO: 2 - protein, 1095 aa, synthetic construct
MAEDQTIGVDIGVASVGWALLNIKKEILESGVCLFPEADAAQNGVRRSMRQSRRLKRRERTRLGDFEKLWLNSLGKYNRVGNDILGIRNKGLSDSVSMDEIHAVLHYMLKHRGISYLEDSIDESSGSDYTKGIMVNQKELEKKLPCQIQAERISQYGAFRGNNTVEINGEKVTLSNTFTTSAYKNELSVFLAKQKEFHPEITDEFVDSYMTIFSRKREYYEGPGNELSRTDYGKYTLKLDANGNYITENNIFEKLIGKCSVYPEEIRASAASYTAQEFNALEDLNNLVVNGRKLDEDEKTKIIKVYKSEDTVSVKKIIKKIIGEEIASLSGFRIDKNDKELFHTMEVYRSMRKAMRDRELDIAVFSRDDIDEIARILTINTEKDGIVKAFNESSLDVDNNIVDFFIEFRKKNGKYFTKWHSFSLKIMNELIPEMYEQSKNQQQLLTDIGYFKRKSDKLKGYDRVPIDIMTEEIFNPVVKRAVATTVKVINALIDKYGYPGKIVIEMPRDKNSDEEKKRIKDAQKTNENELKDIIKKVKNEYGIEITDEMFRHHKGLVLKLKLWNEQQGKCLYSGRTIQIDELINNQNMYEVDHIIPISISFDDSRANKVLVYATENQLKGNRTPFAYLSTVNREWNLEQYISYVKLLKFSSKNKIKNLLYAEDITKIDVVKGFINRNINDTSYASRVVLNLLQGFFAAKEEKTVVSVVRGAFTHQMRVNLNLEKDRDESFNHHAVDAMLIALSQLGYDNYAARQKAIIDFETGEILSEKEWNDQDNLYRQLLYQAEWYGIKNRIAMAEKSIKYHHRVEMKCNKGLCNQTIYGTREYNGKTYTIDHLNLYDDSDVARFKKMIEGGKETNFLMYDNDPETFKALMIVYNQYKDEKNPFVAYHKATDDYLRKYTKSGHNGTIVKKVKFRKEITGSCIDISHKYGFDKGSKKVILDTLVPYRMDVYKNRNDGLYYFVGVKQSDIKCMGRDYVIDEEAYTRTLINERMISEGQSRANLGTLGYEFMISFYKNNIIEYEKGEKIFKERFLSRTMPNDRNYIETKPVVMQNFGKRNLVGLSKTTSVVKVVTDILGNEKRISKEKFSYIVDSH

SEQ ID NO: 3 - protein, 1313 aa, synthetic construct
VENVKKYNIGLDIGVASVGWCVTDMESNILKKGNKNMWGSRIFEEAETAANRRSFRSSKRRLTRRKERIRILQDLLNEDMEKEYPNFFPMLKETSLNYEDKKIDVFVDGKKFNLFSENGNTDKNYYAKFPTIYHLRKYLINTNEKVDIRLVYLAIHHIIKYRGNFLYEGDFSENINAVNEKIQNLIEYLESNYQIIINGKAEDFINILIKKDYSKSRKKELLLQLFNYDKSEKPIIVNVINAIVGSPFEINKIFDTELEKSKISFSTEIEEIESIKDNLGENLEIFNILNEIYSWYTLQDILKGKKFISEAFIEKYNKYSDDLELLKKIYKKYFKSEYSSMFRKDGNDNYVAYNGKSCGKTYKKCAPEAFFNSLKKQMEKLPDNVKEKEIILNGIADNTFLEKINVTENGAIPYQLHMIELEKIVNNQAKYYKTLEENKNKILQLFSFRIPYYVGPLAKKAEGWAWLVRKSDDKIMPWNFGDVVDEDATAEEFIKRMTNKCTYLINEDVIPKQSLLYSYFCVLNELNNIRINNKHIAKDMKLVVIRDLFQNKKKVTLKMLKDYYKKNAFSCDIVTGLQDGENFNSSLTSYIDMKNIFGSVNEQDIEKYEKIIYWITVFEEKKILKRKIKKSFPEITDEQLKKLLKLNYSGWSRLSKKMLEGLKSYDGDNVIEKLEKTSLNFMQIINSEKFGINKKIEELLPVPSKGITYKDIEDIPTSPANKRAIWQSICIVKEIVSIMKTEPSNIYIEFARNEEKKKQMKDARTKQLLKKYEDINNQLRILKEYDSLVYKQLKKHQSDKILTEKLYLYFIQNGKCLYSRKPLNIDELDKYEVDHIIPQSYIKDDSLDNKALVIKRENQRKKDSLLLDDKIINDQEEWWKSLLDAGLISQNKYYKLVRRKMFETEEEKDKFVQRQLVETRQITKYVTNLLINEYEKTEIFAIRSNLTHEFREEFKLYKNRNVNNYHHAQDAYILNVIGNIIKKHMYNHNEFKYSEYVKKHKDENNKNSNVKYSLVMSLIRKYVEQDLVEEVMGYKDCYLSRMLIENTGEFYHQTLYSPSQKPVIPLKKGMPVEKYGGYSNENKAYCSIFKYTNQKNKVEYELIGIPIQISYEIKQGKITLEEYVSKYYLKEKKFYDLVIVKEKILINQEYLDENNNLMRLCSDKEIRSSKELIVDKDISEIIYLMNKDEKHLDDEQIEMIEKEYSVVFDYLVKKLDKEYKIFESTVEKLKTKYKEFENSSSTNKKLIINGLIDLMSTGQGNLKAIGLADREGRKSGQTFKNEKLLNMTFIDKSITGMYERRYKINGMENCSSK

SEQ ID NO: 4 - protein, 1344 aa, synthetic construct
LENNKKYNIGLDIGTTSVGWAVVECENQKIIRKGNKALWGVRLFEEANTAADRRNKRSTRRRYDRRRERIRLLQEEFKEEINKIDSNFFTKLKESFYHEDDKENKTIPLTKEEKETIKEYNNKYKTIYHLRNKLINSTEKEDIRLVYLAIHHIIKYRGNFLYNSDNFSVENLNLETQIETILNSAINFCPSLECPTDYKEYIKINDLSIALLDSSKNDRKVKVQKILESILPKIFVSEFIKLINGNKFNIIKMIGLENIDKLEISFNGTDYEEKYNELESILGDSIEVLDSMKQLYDMVFLKRLFKSSESTNISALMVEKYELHKKDLKFLKSILDIDRKIYNKIFKSRKEICPYENYIKNKITNEEFVKEIKKYLPSIFEKNINQELLTYYEANIQKRMENGDFLPRITDKDNGKYPYQLNKEELIKIIENQGKYYPFLLDKTKDDTYKLVKLLEFRIPYYVGPLTEESHSKFAWMIRKNYNEKITPYNFDDIIDKEATAEKFIKRMISHCTYLLEEPAMPNNSILYSKYKVMNELKQIRINGKKMSCDMQNKILRNLFMTTSGNITDKKFKEYLYANKDFDMYQGDFNITGYSADGKFANTMQSYIDFFGENGIFSNTNYTLEEAEQIIEWITIFEDKDILKTKVERDYPLLNDSQIKKIISKKYSGWGSLCKKLLTTKYYVDKESGVTKSIIDLMEETENNFMQIINDDTYNFQKMIKENNKIDVTQKLNYSVVECLATSPSTKRGIYQALRVVDEIVKYMKKEPEAIMVEMARGDDQKKRKDDRKKYLIKLYEKAKEEIEDYNQLMQQLGKFEKIDSQKLFLYFIQEGKSLYSGKPLNIEDLATYEIDHIIPRTLIKDDSIDNKALVYREENQTKAANYILPREFRSQFNRAWWEHLRKIGLISSKKFHNLIRSEYKEEEIENFINRQLVETRQITKHVANILNSYYKNTQVVYLHADLSHNYREKFELFKFRDINDYHHAHDAYLAAVLGEYKEKYLKCKVNFEMLKELNKSLIEQKDYKKLKYGYVINSLDSSMNDVMKEVVSIHFDEKTGEVLFDAEEFNRRVEDTLYRNDILISRKPEIKTGQLYKETIYSHNDAKIKSGIKISKTLPVEKYGFYTNIEYSYLVLVKVKEKNYMFGIPIMYDNKIDKAKYIEEKLKVSIDDYEIICDRIPFYTKINYHGHLAYLTGSIEFWNAYQLKISKKSMKKWKYMLNFALNDKKIPVIDNQEVLTEQEVNKQMLEFIKYLLSLHELFPLYNNTFKKLEKIDIENLDMEKKKILIKELLKMLSASSMNANLKEFGLTDREGRVHKSNIGEGTLIYNSITGIREKTIVIEGKRK

SEQ ID NO: 5 - protein, 1364 aa, synthetic construct
MNLKKGKKYNIGLDIGTTSVGWAVVEANTQKVIKKGKAKKALWGVTLFEPASTAEKTRSFRGTRRRYDRRRERIKLLQNEFASEIDKVDKNFFKKLKESKYHEEDTVNKSIILTVDEKKKVKEYNDKYKTIYHLRSRLIHDDLKEDIRLVYLAIHHMVKYRGNFLYNVKEFNTNNLDLTTTLKNGFDTLVNLVPHLNIIENYEEMISLEEISNAIMMPSKNDIKVKMKELLDNFSDNKKFGTEFGKLVVGNKFSLKKLLMIEDLEKDISISFDGTDFDDKYNELEDSLGDLIEVVSVLKELYDTIFLKKLFKGNTISSLSDLMVERYNIHKEDLKFLKQLFNKERDLYDQLFRTKTKDKSCFYEQYIHNQLTYEDFIKELKKLLEKISDTNIEDRLRDKYTKEIQPRIDNGDFLPRITDTENGKYPYQLNKDELIKIIENQGKYYPFLFDKINDDYKIVKLLEFKIPYYVGPLVSSDKSRFAWMERNIDKVKITPYNFDEVIDKETTAEKFIKRMVSHCTYLLNEYALPTNSILYSKYKVMNELKQIRINGEKLSKLNEKLPHKIMEELFMKTSGPITDKKFKEYLYYSNELTMYENDLIVTGYSSDMKFANNMQPYIDFFGEKGIFENTSYDLDNADEIIEWITIFEDKDILEKKVRNHYPLLNDFQIKKVLSRKYSGWGSLSKELLTGVTIKDKKSGVPKNIMTLMEETDENFMQIINNDEYNFQKLIKEKNILKDVDKINYDLVKDLATSPATKKGIYQALKVIEEIIDYIGYEPESIMIEMARGEDKKKERKDDRKKYLQNLYQKMKEENRIDDFDDYDVVSQKLNKIEKIDSEKLFLYFIQEGKCLYSGESLNIEDLNSADYEVDHILPRTLIKDDSIDNKALVYRKYNQKKAASFVLPSEYRTGYMKKWWNHLKKNGLISAKKFDRLIRDSYKDEDIQGFIHRQLVETRQITKHVANIISNFYKTTNVIYLKANLSHNYRERYELFKFREINDYHHAHDAYLAAVLGEYKEKFMKKDINFAMVKEMNEKLKALGNYQQLKYGFVINSLDENVSDIVAEISKNIVDKNTGEVLFEAKRFNDQVEKTLYRNDILVSRKTEIRTGKLFKETIYPKGVGNIPIKENMPTSLYGGYSNVETSYLVLVRYGKKKKLMGIPLEIELKSKKDINIKYDFIKKHLKSVDDIEIVKDYIPYETLMELKNQKVYIKGYSISHKNCEVSNALQLKLPSIKAKEWRYVLNKVLNKKNIPMVNGISIVSDELLIKTMKEIILYLYEQKKNYPLFQGEISKIEEKLNLDEMTLDELEKVIIQLFIIYHCNSANGNLKDFGLSDRIGRLSGYNIDNGTIISTSTTGIKERDYEL

SEQ ID NO: 6 - protein, 1104 aa, synthetic construct
MGLRFGIDIGVASVGWAVVNDEYEVVESGANLFPSADASKNVDRRTFRQMKRVHRRRKTRVSDFDKLWKNHGRKLPESNPEEVLELRVKGLTQALTEEELYAVLKNALLHRGISYLDDAGDEAVSGRSDYERGIMLNRQALDENMHPCEIQFKRLKEYGSYRGNLTIDVDGEKVMLSNVFTTGAYRKEIFEILERQQESHAFLTDDFVENYLKIFSRKREYYVGPGNEKSRTDYGKYTTRIDAETGQYVTEDNIFEKLIGKCSVYKDLTRAAGATYTAQEFNLLNDLNNLTVNGRKLEKEEKVQIIEEVKTANTVNMRKILKRVIGEELETLTGARIDKKEKELFHHFEQYNAIRKAFEKQGWDISELSREDLDSIGDILTLNTERESILGGFQREGLTLSEEKKDCLIALRKKSGALFSKWQSFSLKIMQELIPELYAQPKNQMQLLTDMGVFKTNQEKFAEYHQIPDEMILENIYNPVVSRSVRITVKVLNALVKKYGNPSQIVIEMPRDNSEEEEKKRTKEIQAKNEKELEGIVKRISEEYGIKITEQDFHQHKKLVLKLKLWQEQDGICVYSGRAISIQDLLKNPYLFEIDHIIPKSISFDDSRTNKVLVYRTENQEKRNQTPYLYLKNLRRSWGYDEYRSYVLELKRKGLIPKAKLDKLLFTEDITKIEVLKGFLSRNLNDTRYASRMVLNILQSYFKAKKADTTIRVVRGSFTHQLRNVMRLEKDREESYAHHAVDAMLMCYSQMGFDAYHARQKEIIDFETGEILDENKWNQIMSDKAYDELMYQNQWMRIRANIQKAEKEVKYWHKRDSKPNRGLCNQTIRGTREKDGATMKVNKLNIYEPSGWAKLKKMIEQGKQERFLMYLHDRKTWDDMLRIIEEYQDAANPFVEYEKETGDYLRKYAKKHNGPRIRQLKYLDGEVGSCIDISHKYGHEQGSRKVILESLNPYRMDVYYHAGKKAYFFVGVKYADLKFEKGRYILNEEAYQRALVSEKLIAASQNRFDLEELGYRFVLSFYRNEIIEYEKDGEYYTERFLSRTMPKQKNYIETKPMDAPKFEFVDRHFVGLRKTKSVKKIRLDILGNRFYCSQEKFTLKVDVV

SEQ ID NO: 7 - protein, 1132 aa, synthetic construct
MQIYVFKDIPYSGKLYTDKPETKYTLEEMKMGVRFGLDIGVASVGWAVVNDNYEVEEAGSNLFTSADANQNVERRSFRQTRRLHRRRGNRINDFKKLWLNTGLPIPATNIPDMLELRNRGLNGRLSEEEMFHVLKNELLHRGISYLDDATDGSETAQGDYAKGISQNQKELDSGKFPCEIQSERLDKYGKYHGNISVGNGEEKIILSNVFTTGAYKKEILAILEQQKKFLGFLTDSFCEKYIEIFLRKREYYTGPGNEKSRTDYGKYTTRTDETGKYITEDNIFEKLIGKCSVYKHLTRAAGATYTAQEFNLLNDLNNITVNGRKLSKDEKEVIINEVKNANSFNMRKILKKAIGEDIETLTGARLDKNEKEDFHKFAQYNAIRKEFEKNGWDITVLSREELDSIGNVLTLNTEKEAILNGFKREGLSLTEEMENCLISLRKQKSPLFSKWQSFSLDIMLELIPGLYEQPKNQMELLTDMGIFKSNKDIFKNYSSIPANAILENIYNPVVTRSIRITIQIINALIKKYGYPDQIIIEMPRDKNEEEEKKNIAERQKKNQKELDGIIKKISSEYGIKITEQDFHQHKKLVLKLKLWNEQGGKCPYSGRTINIQDLLYNPSLFEVDHIIPKSISFDDSRSNKVLVYSTENQKKGNTTPYNYLRHLSGNWGYKEYFDYVCRLRKDNMINKRKMENLLFTEDINKIEVLKGFITRNINDTRYASRMVLNTLQGYFQAKEAPTKIKVVRGSFTHQFRNNLYLEKNRDESYSHHAVDAMIMCYSQMGFDEYHKLRSEIIDFETGEILDIKRWEEEVNEKTYNELIYQNKMIQIRANIQKAEKKVKFWHKRDSKPNRGLCNQTIRGTRNDNGVIKKVNKLNIYDPSGVETLKKMINNGKQDCFLMYKHDKRTWENMLQIMEDYRETKNPFTEYENETGDYLRKYSKKHNGPRIVKLKYLDGEVGSCIDISHKYGFETGSRKVILESLKPYRMDVYYNEKDQKYYLIGVKYSDLKFNKGTYIINEAAYNKTLVSEKLIKPGQTISDLERLGYKFMLSFYKNEIIEYEKDGEYYTERFLSRTMPKARNYIETKPLDAPKFQKPQKQIGLSKTTSIKKVRLDILGNRYYSGQENFKLTVDIN

SEQ ID NO: 8 - protein, 1111 aa, synthetic construct
MKKYVLGIDIGITSVGYGIIDLETGAFVDYGVRLFKEGTAAENETRRTTRGRRRLTRRRKTRLSDMRKLLQNNHILNENYHPINNVYELRCKGLHQKLTSDELSAVILHITKHRGSILETVEDDIEKSKDAESLKATLQKNEQLVKQGNYICEIQLDRLHSQNKIKGHNNNFSTKDYVNELTEILKHQGLSDDLNKQIINIVKRRRAYYEGPGSEKSPTPYGRFIEVDGQIQQIDLIEKMRGKCSIYPDQLRAPKLSVSAELFNLLNDLNNLSVSGEKLTVDEKTMLLKLANEKGNLTLKQIAKNLEVEEAEIKGYRIDKNDKPLFTELKGYKKIRNIFKKEGVSVSLKDYEFIDFIVEILTNKKGIEERKQALSESHFNITEPLADTLANTTGVSGYHSLSLKALRLLNTELFESTLNQMQLLYELKLFGANKKSYKGKKNIEADIDAILSPVAKRAQNETFKVINALRKKYGEFDSIVVETTRDKNSDERKKRIKKYQSQNEASSKEMNKMLKESGYDPEKINGKTRMKIRLYQQQQGKSAYTNQPLELHQVIKDPSYTEIDHIIPISVSLDDSQNNKVLTLRSENQAKGNLTPFMAFQANKFKYSYAEFKANVLANKNIPYKKRQNLLNEENITKEEVAKKFINRNLNDTSYACRVVFNTLADYFKDNEIDTKVHTVNGRLTDLFRKKIKLEKERDEDFLHHAVDALIVASIKKMNLLNGYLSKCSFDNLYNETTGEIFTVAGDDKFFDERYIRYIIDLKTMYLQSNQYYNHLIERKDMIYPPMKISHKINTKPNRKIADETIYSTRSVDDVDYLIEKIADIYDPKEKKCKTLVEDILNHNEQKYLMAQHDPKTFDLIRQIVEYHYTQYHDDPKMYKTEIKKEKSEIKLIGDNPLTSYREEHGPIRKYSKKGNGPEICSIKYQSEKLGNHIDISANYQVDSKKVILKQISPYRTDFYHCSDGKFKFVTVRYINVEYQKSTQNYVISEKWYEKEKQKKGIKASDTFLFSLHRDELIGIVRQKGKPYYYDLSTEKEGDKMIADGIRPEILKFTATNNDSTNIIEVKPLYTYCNKRLMCSISNSINLRKYATDVLGNLYEVKENRLKLEFD

SEQ ID NO: 9 - protein, 1351 aa, synthetic construct
MKDKEYYLGLDMGTASVGWAVTDENYHLIRKKGKDLWGIREFEEALTAVDRRTKRTSRRSRQRETARIGLLKEYFHDAITQKDEMFYSRLENSKYHIEDKDENVRYKNVLFCDADYTDKDYFKEFPTIFHLRKALLASPDTHAVRQDIRLVYLALLNMFKHRGHFLNTSLSEDGGNRRLSEAYTDLRNMLIEELGISFPEANSDQMLDILTNKELSRTRKKEELAKLLEIERKDKYKMEFVKAICGLKANAKLLFENFEADGNEKIEICFAEYGYEEKAADISATLGETYYGVVELLKEIYDICTLANIMKGYSYLSEARVAEYEKHGTDLKILKRTLKRYRTIKEYNNFFREHKEGTYCAYIGSVNSKKKLRRNIKERKQDDLYKSIKRLLRGLPESDTDVAYILGELDKGSFLPKQLTPSNGVIPNQVHIKEMKKILENAETYLPFLKERDEYGLTISERILKLFSFQIPYYIGPTTEKSKTGWVIRKAPGPILPWNIEEKIDVKKTSEEFVSKMVRRCSYINGEKVLPKASLLYESFCVLNEINTISIDGERIPVSLKQGIYTALYKKGKKVTRKQLVNYLRAQGALSDESQLSGIDVTPNNTLSTYGKFKAILGEKIDEDDGKQMVENIIFWCTIYGDSKRFLRETLSEHYGNVLSAEQIKRIAGIKFKDWGRLSKAFLTLPGCQKSIGENTSLIRMMWETNFNLMELLNHPDYSYHEELDARQENGLKPLSQLQAEDLDEFSFSAPVKRMVWQTLLVIRELENVLGAPPKRLFVEMTRKPDDKKGRTITRKQKFLNLYHNIDKEIKDWKEEIEKADANGTLKSKKMYLYFTQMGRCMYTGEEIELSQLFNDNIYDIDHIYPRHFVKDDNLDNNLVLVKKTVNAHKSDIYPLEASIYETQKENWKRLYHANLITEEKYKRLMGRTPFTDEQKADFIARQLVETRQGTKGVTDILKDLLPNTTIVYSKASNVSEFRQKRDLIKCRSINEFHHAQDAYLNIVVGNVYYVKFTQNPLHFIQKEYSRNAAQYQYNLSRMFDRDVVRGNETAWIGSKNDKTPGTIATVKKMMAKNTPLMTRRNFEGHGGIADETLYGAQKATAEKYIPLKSSDTKLADVTKYGGFSSVSTAYFFLVEHGAANKRTRTLETVPIYMKEKINDKEELLLKYCTDVLKLTNPCIRLPKIKLQSLIKKDGYFMHISGKTGNQIALRNAVNLCLRQEWISYIRKLEKHQDSGGNCLCTEKNIELYLLLLNKHKNSIYAKRPNPFGEKLAASLEKFKGLALSEQCEVLNQILRLTSIGITCADLTLLGESPNSGKMLMSKNIGKSKELLLLNQSVTGIFESRIDLLTV

SEQ ID NO: 10 - protein, 1359 aa, synthetic construct
MNKKYNLGLDIGTTSVGWAVVESDIQKIIRKGKGKNRKALWGVRLFDEAQTAAGRRGFRSTRRRYDRRRQRIKLLQEEFKEEIDKVDNTFFQKLQESKYHENDLANKTIILSTSEKEKIKDYREKYKTIYHLRKELIDDTSKKDIRLVYLAIHHIIKYRGNFLYSGDNFNVDNLNINSALDEFFEALKNYNQVLNIQENYKEIINYNELVKAMLNPSKNDSKAEIKELFSELSSNKNLGSEIGKALVGNKFSVKKLLLLNDVEKDLTISFSDSDYDEKYSELEQTLAENIEIIDVLKKIYDTIFLKKMFRGSDLSLISDLMVKRYKTHEIDLKILKKLFAKERILYNKLLKTKKEMCLYDKYIHNKISYEDFSKELMKLIEKLFDTFEDIDLKEKWLTEIQPRIDTGDFLPRITDTDNGRYPYQLNLKELQIIITNQGKYYPFLLEQISGKYKIVKLLEFKIPYYVGPLNNTTSKKGVLNKNAWMIRKQENVKITPYNFDEIINKDVTAERYIKRMISHCTYLLEEYALPNNSILYSRYKLMNELKQIKINNVRLTIEQQHNILEELFMKTSGTISDRKFKLYLQISKEFDMYGTEINVTGYSADGKFANSMQSYIDFFGNEGIFEDTSYNEDDAEEIIEWITIFEDKDILEKKIRNKYLELNDNQIKKIITKKYSGWGSLSKKLLTELIIKDKRTGTPKNIMKLMYETEENFMQIINNDEYDFQTLIKNCNINNEIKKVNYKLVEDLATSPSTKKGIYQALKVIEEIINYMGYEPERIMIETARGEDTIKVRKDDKKKQLLKLYSKLKEEQELFQLHNLEKVSKELKNIEKIDSQKLFLYFIQEGKCLYSGKPLNIDDLNTYEIDHILPRTLVKDDSIDNKALVYRECNQIKGANFVLPQEYRTTYMKNWWSHLKKIGLISASKFYRLTRDKYTDEDIKGFINRQLVETRQITKHVANIISNLYKNTKVVYLKANLSHNYRERYELFKFREINDYHHAHDAYLAAVLGEYKEKYMKKNINFEMIKEMNLRLKNLGNSKQLKYGFVINSLDENVNDIVTDISNNLIDDTTGEVLFDAKDFNDRVENTLYRNDILISRKTEIRSGEFYNQTKQKKGGNGVPLKSNMSIEKYGSYTSLNPSYAVMVKYKKREQICKRLVGMLIYLEKVSDKERKLYYEKILNLKENSVMDISLKKIPFYSLLCWNGQMGYLVGASSKVEICNAKQFAYNKDFYKSHKNELNHLFNAKNKENENIDYSEKLSEIIDYIVEKIEKEYKLFENLVIELKEIVRHKENFSLSIENKEKIIFQLTKMLNCHSDNANFKFLDSSYSSAFGKKNGRIIENVSVINLSVTGVKRNQNEF

SEQ ID NO: 11 - protein, 1346 aa, synthetic construct
MKREYNIGLDIGTASVGYAVVDKNTHKVIRKGGKKLLGASLFKPGESAKERRTFRSSRKRYNRRRQRIKLLQREFKNEIEKVDKDFYQKLKESYYHPKDEMNKTIVISKNEQEKIKGYHKNYPTIYHLRNKLLTCDKKMDIRLLYLAIHHIIKYRGNFLYTTSDFRIDELNIPKKQQEIFQDLNQLSDKVTIDEDAIETFPYQELEKALLLSSKTDQSKIVPALFQNILPKEIAKEMSNLLTGKKASVKKLFQLDLEEDMKVQLEGDSYEQLHDAIESAIGDLIEILEELKELYNMIFLKKMFKDDHITSLSALMVKKYEKHKEDLKFLKNILQNHSKEYAEMFKNQEKKLCLYEQYVKNKITKEEFRNKTISLVEKIVSKEKKDAIYNDYMTKIKPQLEQEEFLPRITDTDNGKYPYQLNKEELRIIIEKQSKYYPFLLETTSSGENKLIKLLEFRIPYYVGPLNDTTDKKDVKNPNAWMIRKIDKRAITPYNFDEVIDKEQTAEEFITRMIGKCTYLLDPDIKAIAANSILYSKFKVWNELKQIKICENSLSLEMQQKIYKELFLTTKGTITEKKFENYLKKTGEYKMYDQLTIKGYSADRRFANNMNSYIDFFGEGGFFENTEYREEDAEKIIDWITIFEDKEILQSKLEKEYSKLTEKTIKKILNCRYKGWGNLSRELLTNIYYEDQYDGIKKNIMDLLMETNENFMQIISNKKYGFQEQIDQYNSKHHPSKMNYELVKSLVTSPATKKGIYIALKVVEELIDYMGEEPNSIIIEMARGEEKKQRKDARKDYLKKLYQNNKNLISDYKKLMKELNEQDNIDQKLFLYFIQEGKCLYTGTPLEITQLINYEIDHILPRSLYKNDSIDNKALVLREANQRKAASLVLPAEYRNRNNIAWWKRLTEIGLMSPQKFSKLTRDKYSDKDIEGFINRQLVETRQITKHVVNIIKNYHKDTNVICIPANLSSHYREKFKLYKFRELNDHHHAKDAYLAAVLGEYKETYLKKSTDFEKLREQTKKWIDTKKYKELKYGYVINSIDNEVGQFNQETGEIVFDADEFNKTIAKEMYSNQVLVSRKTEIQTGKFYDETKYKKQSDKAKGIPIKTNLKVELYGYYQKPQASYAIVVKYEQKGKTKQQMVGIPIYIDKISKPNDQKRIDYIKELLKLPKEVNLEIVKDKIPFGITLNVDGEICCLVGASDKIEVCNAKEFYIDKENMKKWNQTFHRLFHDAKDAVDNVTYEKHLNEIIEYIIRKIETEFLLYRSSVDTIKEICKINNIEKVSIENKEIIIKELLRLLRYNSETANFKLLDKKWNERFGRKSGKTITHAIIINRSITGIWEQKDEF

SEQ ID NO: 12 - protein, 1206 aa, synthetic construct
MNEKYVLGLDIGISSVGWGLLRLDEENNPIRIIDTGVKIFSPGEVPKTGASKNLNRRQKRGIRRISRRREFRVDRIRYLLNLNGYLGESIVTGKVSEINDELTILYNNMLNDYYKDKNINPYMLKVEALDRKLSKDELSIILVHYAKHRGYKSNREEDSSDSENGKVKSSIEENKKIMEESKYRIISEMFINDNKFKERIRNTNGDYKMCVTREMYLEEIEKVLNAQIKYGLITEDFKNEYVEIWSSQRHYAKGPGGDSKYGGNLIEKMTGKCKFDNKPRAPKCAPSSEIFVALTNLVNFRYKSKRNDEYQNLNSEQIKKIIELAKNQDSITYNKVFKELGIENGIVKGISATSKEKASAINAFKKKNKLTEFEYGALNEEEKNVYDNLKIEEINKREFIKLKSYSFMRKEFIKQLGTLKWEEISENLELLDLIAKILTDYKLDDDVEKAIKDNDIDEIYKDAILALPNFKDHIMLSSDLVRKINAIMIEGITYDEAMSKLGYSHTDLNSEKEKFDLLIPINQDEELNNQRVIRSLSQARGIINSIIKKYGMPERINIETARELAKSREERNEIAKKRDENQEKNKKIKLELVSLGIFNNIENVKATDVLKYKLWKEQACKCAYSLEAIPIEYLFKDNLVQIDHILPYSRTFDDTYFNKTLVLSKYNQDKGSKTPYEWLGKTEKWLEYKGFISNLDISEKKKENYLLLKLTPEIENDFRNQNLNDTKFISKYLVSYIKANLNVSKVGCPSGSITGKLRNYWNLNGLTHSLDSETYYINNRDENFKKNRENNLHHAMDALVIAATNDKVIRRVSNYERYKRYLDNKPLEYIIDYGNKIDDNFDVNQFVDENGELQEGSLKDYIKDLRANEYFIGKHNNTIRTYVPEPYKGFVNEAKIRVYEQNKEIMDFKLKEFNYSDSEINNALPIIPKFAKDKIGGSLHGETYYSLRVRKSGEDEVKSQISRIDLNSDSFDSKKLENILEKGGGSKVVYETIKEWIGEEKGSIAYKKNGGYPINPKTGNMIKKIKIENEYKGKGHLLGEKIVDKADICQIDVYKKNGEEKLYFVAYDALDLYQIKEKKDKCGNIIKKSQNIDVDIWWGQGKNHMMMSFSSLNNEYQKYLSLKKNDLVEIELNNGNKGCGYVTGCTSGMIEIKSPLGDGKDLIGKNLLFQQIRDRYTITISTIKNINKIKISNLGKVEEWDIDKFL

SEQ ID NO: 13 - protein, 1361 aa, synthetic construct
MEKELKKYNIGLDIGTTSVGWAVVEENTQKIIRKGKGNNRKALWGVRLFEEATTAAERRGYRSSRRRYDRRRNRIKLLQQEFEEEINKVDKSFYKKLAESKYQSDDKINKSILLSADEQAKVKKYNDNYKTIYHLRRRLINDKAKEDIRLIYLAIHHIIKYRGNFLNSNENFNTNNLNLVDHIKGCFEAFINLVPTLNIPENYEEIIDLETLATTIMDNNKNDVKINVENSLKNISDNKKFSSEFAKIVVGNKFSIKNLFYLESIDKNDEITINFSEADMDNSEKVVKIEQLLDDKIEVINLLKELYDAIYLKKLFKNSDKASLSELMVARYKTHREDLRFLKDLFDKERNLYNELFRTKNTKKICLYEKYIHNQISYDEFIKELNKRLEKVFESDIDNALKEKYTISIKDRIINKEFLPRITDPENGRYPYQLNKDELIKIIENQGKYYPFLLDKVGNKYKLVKLLEFRIPYYVGPLVSEDKSDFAWMERKIPNVKITPYNFDEVVNKEVTAEKFIKRMISHCTYLLNEPALPNNSILYSRYKVMNELKQIKINGEKLTQDIQHKIIEELFMKTSGSITDKKFKIYLHSLNEFSMLGNNINITGYSSDEKFANNMQSYVDFFGENGIFKDTSYNEEDADEIIEWITIFDDKDILEKKVKDKYKELNPEKIKMILTKKYSGWGNLSKKLLTELSIKDKKTNTSKNIMTLLSETEENFMQIINNDEYKFQDLIKKNNVIKETKKINYDLVKDLATSPSTKKGIYQALKVIEELIKYMGYPPTNIMIEMARENNNKKERTLDKKKYLLKLYDKIKNDYKDIYSELKKVDKIDSQKLFLYFIQEGKCLYSGEPLSIENLNEYEIDHILPRTLIKDDSIDNKALVKRECNQIKAANFVLPEQYRSSYMRKWWNHLKSVNLISAKKFYLLTRSSYNDKDIQGFINRQLVETRQITKHVANIINNFYKNTKVIYLKANLSHNYRERFELFKFREINDYHHAHDAYLAVVLGEYKEKFMKREINFTMVKVMNDKLRSLGNYKQLKYGFVINSLDENVSEIVTEISKNMIDENTGEVLFDAKTFNEQVEKTLYRNDILISKKTEVRSGEFFNQTKEPKGGKGVPLKKNMPIKRYGSYTSLKPIYAVLLKYEKKKKYEQKLVGMPIYLAKSPEDMQTNYFKGLLELEKDSYCEVISKPLPFYSLLNWNNQICYLVGASNKVEVCNAKEFHYHKNFYKSHKHILNKVFNNNKIEIDETVYDNGLDDVIKYIINKIEQEYVLFDNLISELKNIVQYDDMEVLSIEDKENIIIQLTKLLNCKSENANFKLLNSSYSSAFGRKHNKIIDNASVLSLSVTGIKENDYELSDSSNN

SEQ ID NO: 14 - protein, 1334 aa, synthetic construct
MKNYNIGLDIGTTSVGWAVVEEDTQKVIKKGGKRLWGVRLFEEAHVAADRRMKRSTRRGYDRRRERIRLLQEEFREEILKVDPNFFTKLKESFYNELDVENKTIKISSVEKNLIKEYHQKFPTIYHLRDRLVHDTSKMDIRLVYLAIHHIVKYRGNFLYHTENFNVEHLDIEKNLSVLFENLLPILDISLEPDYSMLASAISISSKNDRKLAISECLSGFPKTFVNEFIKVFNGGKFDFVKMLALSMEDAKVKLTFNGSDYEDSYEELEKVLGEYIEILELLRELYDMFFLKSLFRNSEHTNLSSLMVSKYDTHQKDLKFLKHIFCYDEAIYSKFFKTKKQLCIYEKYVHNHMTNDEFCREIKKYLPDVFEKVECSSLIDEYTNSVCIRMDNGEFMPRITDGDNSKYPYQLNKYELIKIIENQGKYYPFLLNKTEDHVEKIVKLLSFRIPYYVGPLVSKEQSQFAWMIRKDSGKITPYNFDQMIDKEKTAEEFIKRMISHCTYLLNEFAMPACSILYSKYKVMNELKQIKVNNEGLTNDFQHKVLNQLFMKRSGAITDKVFKEYLYQSREFDMYGTDLTVTGYSADLKFASNMQSYYDFFGDGGIFEGTSYQEEEAEQIIECITIFEDKEILEKKVKTNYPELTTEKVGQIVRKKYKGWGSLSRKLLIEKYYVDPTCGTKKSIFDLMYETKKNFMQILNEDQYQFQKMIDDYNQVDSNQKITYALVENLATAPSIKRGIYQSLLVVEELVKYMGKDPSSIVIEMARSHESKVRTNDRKKYLTQLYENSKEGIEHYNELMAELQKVEQIDDRLYLYFMQWGKSLYSQTPLSIENLGEYEIDHIIPRTLIKDDSLDNKVLVLKEENKNKAASFVVPACYRNSVNQKWWEQLKKMGFLSFKKLHRLSRKYYTDQDIEGFINRQIVETRQITKHVANILNHYYPSTKMIYLKSKLLHDYRKRYQLFKFREINNYHHAHDAYLAAVLGEYKEKHLCRKIDYDIVKTLNAKLFASKDYERLNFGFVINSLDSSFNQVLLPLTKNVVDVKTGEILLDVDEFHKRVENTLYCNDILISKKVEFRTGEFYQQTKNAKGMSGVSLKKNLSTKLYGSYTSLHPAYAILVRYTHKGKEEQRLLGLPIYFLEQSKTDTSAISDYVRRTLKLTDHDTFSIQKERIPFYSLLNWEGQICYLVGASDKVEVCNAKEFYFDREFMKKYKTTLEKLYHQHRLEVEDYDSNLESVICYIVDKMEKEYKLYENLIVQLKHIVCYDHLVDLSLEEKEKIIIELLRLLKCDSANANFKFLDSSYSSAFGKKNSRIISSAIIKYHSVTGIRGRHYEF

SEQ ID NO: 15 - protein, 1367 aa, synthetic construct
MVLRQFHSNIVVGGVSMKNKKKYNIGLDIGTTSVGWAVVESESQKIFRKGNQALWGVRLFEEANTAAERRAFRSIRRRYDRRRGRIRLLQEEFMTEIDKVDSTFFIKLKESFYHESDKNNKTVLLTKREKEEIKEYHERYKTIYHLRKELITSSNKQDIRLVYLALHHMIKYRGNFLYHVDGFNIENLDLNEQVKIVLNSITNLCSSLGCSEDYDNYLDIKKFTELLLESSKAKRKEELQKILKNLFPKNFINEFIKLVNGNKFNFIKMLNLEMEESRIELSFDGTDYEEKYVVFESILGEHMEILTAMKQLYDMIFLKKLFHGSKSISLSSLMVEKYVTHEQDLRFLKDLFQIDRKIYNKIFRSKKDSCIYEKYIKNKISNDEFIKEIQKYLPEIFDKSGDRKLLDTYELEVKNRMENGEFLPRITDTENGKYPYQLNKDELIKIIENQGKYYPFLLDKLEDGTYKLVKLLEFKIPYYVGPLTDETHSNFAWMIRKNNTERITPYNFDKIVDKEATAEKFIKRMISHCTYLLEESAMPNHSILYSKYKVMNELKQIKVNGERLTNNFQQKILKELFMKVSGGITDKKFKEYLYASKEVTMYQGDISVTGYSADGKFANTMQSYVDFFGENGIFDGTIYNEENAEEIIEWITIFEDKDILETKIRKKYSKLNEIQIKKIIAKKYHGWGNLSKKLLTTKYYVEKESGIPKSIMDLMTETKDNFMQIINNEDYHFQKMISDYNKVDTTKKIDYSFVENLVTSPSIKRGIYQALKVVEEIVKFMGMQPQAIMIEMARGGAKKERRDDKKKYLTKLYEKEKDSIHDYYQLMKELGEFEKIDSQKLFLYFIQEGKSLYSGRPLNIEDLDSYEIDHIIPRTLIKDDSIDNKALVYREENQIKAASYILPSKYRTEINIKWWQHLKSINLMSQKKYYNLIRKQYKTEDIDGFINRQLVETRQITKHVASILGNYYNKSKILYLPVNLSHNYRKRFDLFKFREINDYHHAHDAYLAAVLGEYKEKYLRKKVNFEMLKELNKKLLEQKDYNKLKYGYVINSLDYSMNKVLKSMILSESNKEVGELSFDAEGFNKRVEDTLYRNDILISRKTEIRTGEFYNQTKNKKNLPGVCLKKNLPTKLYGSYTSLNPAYAVFVKYTKRGKKEQRFIGMPIYIIESSKSDLSIQSNYIRDLLKLNENDVLEFDDRKIPFLSLIDWDGQICYLVGAADTIEVCNAKQFKFDKNSMKRFKYTLNQVFNVKNRMDTLNEEEYEKNLGELIRYIVDKMEKEYSLYQNLILNLKNIVCYDYPEMLSIEEKEQSIKELFKLLNCRSTTANFKFLNSKYSTAFGRKNQRVINHAKVFNKSITGIRESVYEF

SEQ ID NO: 16 - protein, 1112 aa, synthetic construct
MNSYVLGLDIGITSVGYGVIDLKTNNFVDYGVRLFKEGTAAENEARRSARGRRRLLSRRKNRLEDMRNLLKKYQIIDENFKPLSNVYELRNKGLSTSLTNDELASALLHLTKHRGSVLDTVEDDEEAAKDSEKTKAVLGTNAKLLAQGRYVCQLQLERLQSEGKVRGHANNFKTTDYIEEANEILKHQNIDEKLKEKIIEIIQRKRAYYEGPGSEKSPTPYGRFIEVDGQIQQIDLIEKMRGKCSVYPNESRAPKMSLSAELFNFLNDLNNLTVDGEKLTTEEKNQILKVVSQKGSITLKQVAKLLEVEEDSFAGYRIDKSNKPLLTEFKGYKKVKKIFKECGEDISFHDYEKIDFIIEIITKQKGMQERKTSLENSKYTFTNTLLDKLVLMNGVSGYHALSFKALRELNKELYMTDMNQMQILHQMNLFDKNRISHKGRNNIMADETAILSPVARRAQNETFKVINELRKKYGEFDSIVIEMTRDKNSVEQAKKQRESQRYFEGLNKEVNRILEEYGYDADKVNGKTKTKLRLYMEQDGKSAYTLQPLDIRRIISDPTYTEIDHVIPVSISLDDSLNNKVLITRMENQLKGQMTPVEAYQKGKFVDTGCTLVEYIESVKNNKKYNNKKKGNLLFKEDITKFSVIQQFINRNLVDTSYACRVVLNTLSDYFKDNGIDTKVHTVVGKLTNKLRQQIQLEKIRDEDYLHHAVDALIVASIKKMGLLNSYLMKYTFDKLYDEMTGEVKEVPEDKAFFDEKYISFIVNLKTLYMQSNQYYRGILERKEMVYAPIKISHKINTKPNRQVADETIYSTRNIDNKEMIVQKYKDIYEPKFDKLTNDILSGVTEQKYLMARNDPQTFDILKQIIMHHFDEFKDSKEYYKKEVKKGVVTYKLVGDNPLYLYKEEFGPVRKCSKKGNGPVITSMKYVDGQLGSHIDISKNYDVKNKKIILKQVSPYRTDIYLCSDGKYRFVTVRYKDVFYKKEKQKYVIDANWYAEEKKRKKIDDNAKFICSMHRDELIGIVRKEGDKYIYDESTEHGGEVKYHDGIHSEILKFTATNNDQNGIIEVKPVFTYCKKQLKPAILNCVEVSKYATDVLGNLYKVKQNILKLEFE

SEQ ID NO: 17 - protein, 1313 aa, synthetic construct
MADNLKYNIGLDIGTSSVGWCVTDEENNIVKKSGKHLWGSRLFDEGQTAAETRTFRGTRRRTERRKNRIRYLQSMLLEDIEKVDENFIPRLQQSNLIKDDKNQFKFNLFEDEDFIDEDYYKQYPTIYHLRKALATEDKKFDIRLVYLALHHIIKYRGNFLTKGEISNETNAINLDLENIIEYLKENEIELKYSIGKIKETLVNKELTKSEKAKEILSLFDYGKEDKQIIDNIFKSVVGLKFNLNAIFNLEDERKLEFASDNDWDSIAGELNENAEIFYSMKNIYSWIIVESFLDYEDNSDEKEDKTISKAFVIKYDKYHEDLVKLKNAYKKYLDIIQYDKMFRKYEHDNYYSYNGKLSEIKKSEKCSNEELCERIKEDLKGFEDKSDVSEIFNDISNENYLIKLNTTDNGEIPYQLHEKELKKILENQSKYHDSINENKDKILSLLTFRIPYYVGPLSTKSKWAWVKTNSKEAIRPWNFDEIVDKDATAEEFIIRMTNKCTYLLNEDVIPKESLLYAKFSVLNEINNIQVNDKRVSKDMKTKIIELFKNKKSVGKKDIKNLYAKDGINAEKIEGFTTSNGKFNSNMNAYVDFSKIFDNIDENEEMIEELIYWITIFEDKSILKHKIEEKYSSILTKEQINKIIKLKYIGWSRLSKKLLIGLKSTENNETIIEKLMNTNMNFMQIITSDKFGFKEQLEQALKEVIGEEKKKISYDDIAEIKTSPANKKGIWQAILVVKEITKIMKHDPENIYIEFARSDEEKGTLTTKKLEKVKKIYDEIENFKQANKALYEDIKKHQGDKNINERLYLYFIQCGKCLYSDTKLDIEKLSLYQIDHIIPQSYKKDDSIENKALVLSSENQRKKDSYLLTDEIIRKMTPTWNYLLDKGLISEIKYKKLIKRKMFSNEEEEVNFIARQLVETRQITKHIRGLLSKAYPKTKIQGIKATITHDFRSKYKIYKIRELNDCHHAQDAYIMCIVGNVLNAADFFEYERMKIFLKNRKKKTAEERNEQLKEEHGAIMSIIQNKIDINKVKEIINYKDYLVTRKLEEQTGEFYNQNLLKKIDEPKIKSKTNKDTLKYGGYTNENEAYFVVYEYYNKKGNKEYQLLGIPIKYSYDIKNGKTTIDEYVKNIAKLQEQTFIRILKEKMLKNQAYLNEFNENELLTSSKEYKNAKQLYLNTKEIEKMYIVLDSKNKKQLKQEEIEANSIEIYEILLNKIKKEFKTFNTFDKKKEYEKLDLKDRRIVIINLLRLVNRNHGDLRKIGLSDREGRKRNNSGFNKKKLDNMTFIDQSVTGFYERRYRIDELANSNNKKTM

SEQ ID NO: 18 - protein, 1205 aa, synthetic construct
MKYDYILGLDIGISSVGWAMLALNEENKPYRIINAGSRIFSPGEVEKTGASRAQARRENRGSRRLIRRREFRIDRVRNLLYEEGYLSGNVTSDVVSIKNNELTEIYIKMINNYYREHETNPYKLKVEALDRKLSNDELAIILVHYAKKRGYKSNREDKSENDTGKVLSAIDANKDIMAEKRYRTISEMLIKDEKFSKKIKNSPNNYQMSIENEDYLKEINLVLDTQIKFGTITEEFKNKYLEIYNSRRSYAKGPGGDSKYGGNLIERMVGKCKFDNQPRAPKYAYSSELFVALSKLVNLRYKESDGEYKSLTAPEISKIVDLAKNKTQVTYLDVQKVIGQDIKIKDLSLNIKEFKKVIENLYKVLDIPSSEKIDIKELNEDDTKKYLTLYNEQLMSKNFIQLKGYHDLRKAITKSFGKEEWNNISSNISALDDLVLFSTNYKDNTDIKNAIKNSSNLDDKYSDNNFVELLENYKDHLMLSTDIIRKLIPIMEQGKTYDKAMEELGYNHYNLTENIEKKSLLVPISKCDNITNQRVIRSLTQSRKVINSIIKKYGTPKVINIETARELAKTMDERNKIAKAQAERQGDNERIRQELFEKGFFTDPRKITSTDILKYKLWTEQKEYCPYSLKKITLEDIYVDNLVQIDHILPYSRTFNDNYLNKTLVFTKYNQEKGNKTPFEWLGNGLKWEEYKSFINSLTISPLKKENYLLKDLSLDVEREMREQNLNDTKYISKELYSLIKAYLDVPNVNVYQGAITAKLRARWGFNRLTHSYISETYNMPSEMKQGIQKDRDNHLHHAMDAIVIASMTKSLQNKITLYEKFSRYIDGLTKNMLLNLNSDELKETIGEFYDDETGEVKEIDLKEYLKKQFAANNITFLNHDIGRLKFPVPYETFVEEAKIRVYEQNEEVLKFKLKGLGTYTNEELKKVHVLTPSFAKSKISGAMHKDTLVGLKQFNNDDSNITYKTSRIPIEKVTPKNLESLIKCNPSGGTKDIYEAIHEWLGSSSKGEDALKEHNGKYPVSKNDKENKEIKRVKIYEEYKNTGHNIRNANVDKGDIYKIIIYKSKNKEDNKLYAVGLDIFDIKKIEKYNADKLKNDFNLKLEYGQGSNFIMKKYSEVLSEYEEYIVLVKNDLIKITCGSKSSIVYVNSCANGQLELKSPLGDGYDIIGDQNIFNEIVKNYKKTISTIDNIQKLSINTLGEISGL

SEQ ID NO: 19 - protein, 1354 aa, synthetic construct
MKKYNIGLDIGTNSVGWAVVESEKQKVLRKGNKKLWGVRLFDEAVSASSRRNFRSTRRRYDRRRYRIKLLQEEFQNEINKVDPNFYQIMKESFYNKNDKVNKTIELNKEEKDFFNSYQKNYPTIYHLRNALINEDKKFDIRLVYLAIHHIIKYRGSFLVQGEFNTESLNIEESFHNVVSLICDYCQEFDIDENRDFKDLEKILLLDSKNDKKVELTSYFNFMPKEFTNEFIKMINGNKFNFIKMFLFDIDSAKIELSFSTTEYDDKYDEYEKILGDRIELLEALKNLYDIVYLKKIFKESKTSSLSNLMIGYYDKHKENLKFLKQVFNVDRTIGGKKESLYDKLFRTKNNAKKENYCLYDRYITNGITYDDFQKELKKLFDSVNWNNVDSYLVKKYNEEILMRLENGDFLPRLTSVENGKFPYQLNKNELVKIIENQKKYYPFLGDEYNGTNKLVQLLEFRIPYYVGPLTTTEKSNFAWMIRKSGQESIRITPYNFDEVVDKEASAEEFIKKMISTCTYLLNEKAIPANSILYSKFKVLNELKQIKINDNPLSKELQEQIYNDLFLNTSGTITDKKFKEYLYANKVTDIYGDDILVKGYSADEKFANSMKSYVDFFGIDGVFENTNYTLNDAEKIIEWITIFEDKNILKVKLNREYPDLNEEKVNKLLKTKYTGWSSLSRKLLTGVLYFDKETNTSKSIMDLIIETKENFMQILNNKKYKFLELIDKINGEQITKKVNYDLVRDLVTSPATKRGIYQALKIVDEITNYIGYDPEYISIEMARNEDKKERKPDRKKYLESLYKEYKNKIDNYNDLNKELKNQEKIDSEKLFLYFIQEGKSLYSMTPLKIENLSDYEVDHIIPRTLIKDDSIDNKALVLKKENQNKAASFVLPSNYKTDTNRKWWSRLKSIGLISNKKYYNLIRKEFNSKDIEGFINRQIVETRQITKHVANILANLYENSKVIYLKANLSHNYREKFELYKFRDINDYHHAHDAYLAAVLGEYKEKYLRKNIDFDMVRELNKDLIANKDYKSLKYGYVINSLDDKMNYLINDIIPMQVDEKTGEVITKEFDSKIFNKNIENALYSNDILISKKVEYKSGEFYNQTKQRKGSKGVSLKENLPTKLYGSYTSLNPAYAIMVTYLNKGKETKKIVGMPIYVLSTSKKDETKINEYLVNLLNLKNNKDILKISRPIPFYSLLDWNGQICYLVGASDKVEVVNAKEFKFDKDFFIKHKYTLNKLFNNYKAEIDNYDNNLNEIIKYIVDKIEKEYVLYNNLVSELKSMISYDSLDINSTENKEKIIIELLKLLKCNSANANFKFLNDKFSTAFGKKNGRIIENVKVKSNSYTGIWSKYNEF

SEQ ID NO: 20 - protein, 1311 aa, synthetic construct
VKNNQSYNIGLDIGVGSVGWCVTDEENNILKKSGKNMWGSRIFSEANTAQERRNFRSSRRRLDRRKERINILQSLMQQDIENEYPNFLQMLKESSLDFEDKTSSISINGIKYNIFSNEKYTDQIYYKQFPTIYHLRDYLIKNKEKVDIRLVYLAIHHIIKYRGNFLYEGDFSDDTTEIIDKIDILTNFLREKYNINLKQDIEKIIDILKQKSMSKANKKDKLISIFEFDKSDKPIIVNVINAILGYAFDLNKIFDINLSKNKISFASDIENEEEIKQQLQDDELIYDCLNETYSWFILQDILKGKTYISEAFKEKYYKYAKDLKKLKAIYKEYFPQKYNKMFKSYDKNNYVAYTGKCSGKTFKKCKAEEFFLKLKKDLDELPDTCVEKEEIKKDIENSNFLRKINVTDNGAIPHQLHQKELEIILNNQSKYYKTLEENKDNILKLFCFRIPYYVGPLAKDNSNWAWVIRKNNDEIRPWNFEDVVDEDATAEEFIKRMTNKCTYILNENVIPKQSLLYSRFCVLNELNNIRINNHHIVKDIKKLIIERLFKTKKKITRKMLVDLLTKEEIKIESLTGLQDGENFMSNMQSYIDMEKILGKIDESNIEQCEKIIYWITIFEEKKILKRKIEKEYPKLSLEQINKLAKLKYTGWSRLSKKLLVGLKATDGESIMEKLEKTSLNFMQIINNDVYGFNKQLEKLMPQKSGGVQYNDIAEIPTSPANKRAIWQSISIVKEISKIMKQNPKNIFIEFARSEEMNKSMKDTRVKQLLKKYEEVEKQLKDLKNYDKKVYEELKQHQSDKTLTEKMYLYYIQNGKCLYSGKALNIDELSQYEVDHILPQSYIKDDSIDNKALVLKEENQRKKDNLLLLDEIINNRTQWWKSLLENGLISQTKYYRLIKRKMFETDYEREKFVQRQLVETRQITKYVTNLLKNEYDTSEIYAIRADLTHGFRDKYKIYKNRNINNYHHAHDAYILSVIGNTLNKYWNGTGEFKYSQYVKKYVEDDKNKYEKNGMILGFINKYIHIEKIKSVMEYKDCFVSRMLEEGTGAFYNQTLYSPKDNPTIQLKKDKNVLKYGGYSGAQKAYCIIFKYIDKNNKEEYQLIGVPIQVSYEIKLGKLNLEEYIIDTYLKEKKYSNLKIVKKKILINQEYLDDNYEPMRLCSDSEIRSNKELVVPHKISELVYLMNLDENRLNDDEKEKLSKGYSIMFNYFLEKLKDEYKIFESIYLKLIDKMLKFEELKEVDKKSIINGLISLMETGQGNLKSIGLTEREGRKSGQKFKTDRLSKMIFIDKSITGMYERRYKIGGLENSCS

SEQ ID NO: 21 - protein, 1354 aa, synthetic construct
MEKDYNIGLDIGTTSVGWAVVETNTQKIIRKGKSNHRKALWGVRLFEEATTAAARRSFRSSRRRYDRRRQRIKLLQEEFKNEMNKVDSDFFRKLVKSKYCEEDKTNKSIILSADEKRQVKEYNDKYKTIYHLRNRLISDSSKEDIRLVYLALHHIIKYRGNFIYSGDNFNIDSLNLSEYLTGVIESLINYIPLFNIPNNYREIIDYKEFESIVLLPSKNDIKVKIKDFFKNIIDNKNLGSELGKLLTGNKFSVKKLLLLEELENDISISFSEANYDEKYEELSQALGDKIEIIDTLKQLYDAIFLKKLFKGSDNASISDLMVKRYKAHKEDLKFLKQLFDKNRNLYNKLLRTKKELCLYDQYIHNKINYEDFIKELVKLVESLFDTLEDNELKEKWLIEIKPRIDSGDFLPRITDTDNGRYPYQLNLKELNAILENQGKYYPFLLEKVNNKYKIQKILEFKIPYYVGPLVSEDKSNFAWMERKVNNVKITPYNFDEIVDKASTAEKFIERMISHCTYLLNEYALPNNSILYSRFKVMNELKQIKVNDRKLEIDLQHKIIEDLFIKTSGTITDKKFKLYLYTLPDLDMYKGDINITGYSADMKFANNMQSYVDFFGENGIFNNTSYSEENAEEIIKWITIFEDKDILEEKVRSKYTELDDEQIKRIVLKRYSGWGKLSRKLLKELTIKDKESELPKSIIQLMYETEDNFMQIINKDEYGFQELIKSNNIYLETTRVNYKLVENLATSPATKKGIYQALKVVEEIIDYMGHEPKNIMIEMARAEEQVKARKDDRKKQLLKLYEKIKETQGELEVSNLRKVSEELKSIEKIDSQKLFLYFIQEGKCLYSGKPLNIEDLNEYEIDHILPRTLIKDDSIDNKALVYRECNQKKADSFVLPENYRTTYMINWWRHLKKNGLISASKFYRLTRKEYNDEDIQGFINRQLIETRQITKHVSNILSNLYKNTKIIYLKANLSHNYRERYELFKFREINDYHHAHDAYLAAVLGEYKEKYMKKNYNFEMVKEMNHRLRSLGNYRQLNYGFVINSLDENVSDIILELSKNLIDEETGEVLFDARKFNERVENTLYRNDILISRKTEIRSGQLFKQTIYSKGIGNIQLKKNMPTKLYGGYLNVETAYLVLVKYKKKKKLIAIPLEIALKSEHSNEIKIEFIKKHLNVDNVEIIKDFIPFETLIDYKSQVVYVKGYGVANKVCEVSNAFQLKMPFLHTKRWKYVLNKVLNKGSIPVVNTKPILSEEEILNVMKEIITYLYEQKSNYPLFENEICKIEKQLILNEMNYEELAKAIIELLKIYHCNSVNGNLKEFGLGDRIGRLSGKNIDKGIIITKSITGIKERRYEF

SEQ ID NO: 22 - protein, 1353 aa, synthetic construct
MKNGEYYLGLDMGTSSVGWAVTDDSYQLLKVKGKDLWGIREFEEAKIAVDRRTHRISRRRRQRETVRIGILKEYFNDAIMKADPCFYQRLDNSKYCLADKELSVRDKNGIFHDANYTDREYYAQYPTIFHLRKELLESDQPHDVRLVYLAVLNMFKHRGHFLNASIGTASGERKMSEIYGEFCSLTLEWMDVAFPELDDCSMIENILSMREYSRKRKAEKLADIFHVSSNEKKKKALVSGICGLKMDLKLLFGDSIEYDADKKMAISFSDAGYEEKTQEILEVLGDDRFELIQTMKEMYDIGSLASIMKGYTYLSEARVAQYEKHGVDLKILKALVRKYCDKETYDRFFRKAENGTYSAYVNSTNTGKSLSRDMKGRTREDLYAAIRKLMNHMPAEDEKVQYVREEMEKESFLPKQLTASNGIIPNQIHLAELKKILENAQGYLLFLQDVDESGLTVSERIIKLFSFQIPYYLGPLSEHSAENGGNGWVVRRESGQVMPWNLEEKVDVKETAEMFITRMVRRCTYMNGENVLPKASLLYEKFCVLNEINNIKVYGEKIPVSVKQDIYNELFKRGKKVTRKQLYQYLLGRGLVKEGEIEQITGIDITINNYLATYGKFKNILGEGFDTDDGRNMVEKIVFWCTVYGDFKKFLREQIEEKYGEKLNQEQIKRITGLKFKDWGNLSKTFLEMQGCSKSTGDILPLISMLWETNDNLMELLAKDAYTYREELEKYQGDSYKVLAELQAEDLEDMYFSAPVKRMVWQTILIIKEVEKIMGAPPRRLFVEMTRSGEKEKNRKDSRKQQFLELYKNIKDEEHDWKSIIVDADEKGTLRSKKMFLYLTQRGRSMYTGNPIDLDDLFNDNLYDIDHIYPRHFVKDDNLANNLVLVEKELNAHKSDTYPLEHDIYTSQLPMWRELHRQHLISDEKLKRLTGRNPFTDEQKAGFIARQLVETSQGTKGITTILQHALPATTIVYAKASNVSEFRQKRDLLKNRTVNDFHHAQDAYLNIVVGNVYFVKFTQNPLKFIQKEYEKDRDKYNYNLSRMFDWDVKRGDEVAWVAQHGKEESGTIVTVRKMMEKNTPLLTRLNFEGHGGIAEQTLYSAKKASGVGYIPLKEKDTRMQDVTKYGGFNGVSTAYYILVEHGKTGKRIRTLETVPVLWKDRIEKNLQLLQEYCEKELQLTEPSVRMKKIKMQSLVKKDGYYMHLSGKTGNQIYVRNAVPLCLKQNWMAYLKELEKGERNEMVSVEKNMELYDILCGKHSEGIFKRKPTPMGKKLQNGRVVFERLSCEEQGKVLIEMLNLTKIGEKFANLSLIGDVAKTGVMRIGKKISDAEEFVLINQSVTGVYENRIDLLTV

SEQ ID NO: 23 - protein, 1359 aa, synthetic construct
MQETGKDYYLGLDMGTNSVGWAVTDENYVLMRAKGKDLWGIREFDEALTAVDRRTHRVARRRRQRETARIGLLKEYFHDAIAEVDPDFYQRLDNSKYHEEDKDSAVKGKNGIFNDANYNDKDYFKQYPTIFHLRKELIESTGKHDVRLVYLALLNMFKHRGHFLNAGLSTESENTMDTAYHNFVETAAQTIECNFMETVDIEKIKEILGSRDYSRSKKAELVAQLLHVDSKNKVQMACIKCICGLKVTAAAIFGDKMAADEEKKTDICFSDFGYDEKVPVILEIVGEENFELVLAMQEIYDIGSLAGIMKDSLYLSMARVKEYEQHGKDLRILKGIVKKYGTKEEYDTLFRTMEEGTYSAYVNSVNTKKKSRRDVKKRTRDEFYGRLKKIISKMPQDDSDVLYIKDKMEKEAFLPKQLTASNGVIPNQIHAREMKKILQNAEIYLPFLKEKDTSGLTVSERILKLFSFQIPYYVGPVSEKSKLNGGNGWVVRKEEGTVLPWNIEEKIDMTATSREFVERMVRRCSYLSDERVLPKGSLLYERFCVLNEINAIKVDGVKIPVEIKQDLYSKYFKKGKKVTKKKIAEFLIARGVIKEDQTELISGIDIAVNSTLSTYAKFYAIFGEKMEQDSYKKMVEQIVEWCTIYGDAKKFLEQKIKECYGNILTQEEIKRITGFKFRDWGRLSKEFLELSGCSKATGEIMPLISMMWETNDNLMELLDDNYYTYREALEEKQNNAFKLLADIEPEDLDEMYFSAPVKRMVWQTLCIIKELEKVLGKAPKRLFVEMTRNEQKDKKRTDSRKQQFLDLYKNIKDESRDWKELITRADENGTLKSKKMYLYLTQMGRCMYTGHPIDLEDLFKDNRYDIDHIYPRHFVKDDNIMNNLVLVEKEKNAHKSDNYPLEEEIYKKQLPMWKDLHQRKLITDEKFKRLTGRNEFTDEQKAGFIARQLVETSQGTKGVTTILERALPDTKIVYAKASNVSEFRQKRDLLKSRVVNDFHHAQDAYLNIVVGNVYFVKFTQNPLHFIKAEYDKNKAQYEYNLSRMFDWDVRRGNETAWIGCKKGEEAGTILTVKKMMAKNTPMLTRLSFEGKGGLTQKQTLYSAKQAKEDSYMPLKSSDYRMLNVKKYGGFMATSTAYFFLVEYEVDGKKVRTLEMVPNTWKDRIEKNENMLLKYCTEVLKLENPSIRLKKIKLQSYIKKDGYFMYLSGRSEDRITVKNAVQLCMNSFWMKYIREVEKTVEMKRITEKISYEENMKLYKELCEKYNNGIYSKRPTAVGEKLKGAEQAFAKISLYEQCIVISQILALSRIENPSADLTLIGLSKQTGTMKISKNITNCKEFILINQSVTGLFEQKVDLLTV

SEQ ID NO: 24 - protein, 1352 aa, synthetic construct
MNERYYVGLDMGTASLGWAVTDEKYRLIRRKGKDLWGVRLFDTANTAAERRTNRVAKRRLARERAREGYLRMVFAPAIEAVDPGFYQRLDDSKYCEEDKIVKQPFALFAGNGYTDKDYYEQYPTVFHLRKELLTSKEPHDVRLVFLALLNMYKHRGHFLNSNIGEGSTGSIEEMYENLRSDISQYMELELPDIDDYDKLKNILTSKEYSASRRVELIIESLNLSKSKDKNIVEIWKLTCGLKGKLSNLFAVNTEDDEAKNFSISFRDGSFDEKVTVVENILGEEQFDIFMLIKEIHDHCVLANIMKGYEYLSFARVDSYEKHGKDLAILKKLIKENAPGEYCGMFREMANDNYSAYVGSVNYKNGPVRRGAKCDRDAFFKKVSTLVSKMPESQDREYVLQEISKETFLPKQLTASNGVIPNQVHKKEMKKILKNAEGYLPFLLDKDESGYTVSERIIKMFEFQIPYYVGPIAEKVGEDSKSKNVWSVRLESGRVYPWNFEQKIDVKESAEKFISRMVNKCTYLNGEQVLPKNSLLYERFTLLNELNNLRINGEKISVELKQNIYNDLFKSGKKVTARKLKEYLKTNGYVEAKEDPVISGIDGDFTNRLANYGKFAAVFGVETLTYDQEKMAEDIIKWSTLYGDTKSFLKQKITENYGEDKLTKDQLKRILGFKFKDWGRLSKALLETRGCSKDDGVIDTVIGRMWNENYNLMELIESRNFTYKEEISERSMAVDKLLGEIKYEDLDELYISAPVKRMTWQTILALKEICKVMGYGPSKIFVEMARDPNAEKKRTESRKSKFAYLYRSVKDDTFDWQKAIKETDEGQFRSKKLYLYYTQKGRCMYTGERIELSDLFKDNLYDIDHIYPRHFVKDDSLDNNMVLVKKEKNAHKSDTFPIEADIRGKMSGFWHALADGGFITKEKLKRLMRTDGFSDDERVGFINRQLVETRQGTKVITDLFKQTFPDTDIVYVKAPNVSDFRHEFDLIKCRMINNFHHANDAYLNIVVGNVYDVKFTRNPYNFIKDYEKGKEKYNMAKMFKYNVSRNGVTAWSIDGEKSIDTVRKVMARNTPLVTRMNYEAHGALYDQNLVTKRGITKANRDAYLPAKATDDRLAAVEKYGGYRKYTGTYFFLVEYTEKGKRVRSLEALPLYLKDHYNTKEQLEEYCRERLGLKEPSVRYRRIKMYSKIRVNGFEVLLTGRSGKQLVISNAQELKISYEYAKYIKKLAEFKEKDVQEEIYEENGITREENKKVFLVLKDKFVKEIYSKRVNPIGNRLDEDKFDGLSVKAQAEIILQVVGITQQVNYGADLSLFDESKTSGVMFLNKMISKQREVVLLNQSVLGIYEERVDLLTI

SEQ ID NO: 25 - protein, 1112 aa, synthetic construct
MSKLVLGLDIGITSVGYGVIDLDENSFVDYGVRLFKEGTAADNETRRAKRSHRRLLSRKKTRLADLRKLLKAYQIIDDEQFESLDNVYEIRKLGLTSQLTNEQLASAMLHIAKHRGSSIDTIDDQKENEKEAEALKDTLSKNAILLSSGKYVCEIQLERMKEDGKVRGHQNNFKTSDYLNEAKEILNHQQIDENLKTQILEIIARKRAYYEGPGSFNSPTPYGRFIEVDGQIHEIDLIEKMRGKCSIYPNELRAPKMSITAELFNFLNDLNNLSVKDHKLDIASKKEILMAVSQNGGITFKQLAKKIGVNEEDIKGYRIDTKKKPLLTEFKGFKVMRKIFSQCSQIVSLEDYEMLDDLIEIITKKKGIEERKEAFVQTGYPLSDELIEKLAVVSGVTGYHSLSLKAMRELNTEMYETTFNQMQLISQMENNSQKQVSFKGKKEIMADETAILSPVTKRAQREAFKVINALRKQYGEFDTIVIETTRDKNSEERKKRLRETQKFYEQQNKQVDELLKERGYDLTKLSGKTKTKVRLYLQQEGKSAYTLKPLDLDSVIRDETYTEIDHIIPISISLDDSFNNKALITHGENQAKGNMTPYGAYQNGKFADSGCDYAKYNTFVKNAKNIPYRKKQNLLFEENINKFSVIQEFINRNLVDTSYANRVVLNTLTQYFKDNEIPTKVHTIRGALTDKFRTQIQLKKDREEDYLHHAIDALIVASVKKMNLLNGYLSKYSFNQLYDETTGEVLEIPNEKAYFDTKYIQFIVGLKNLYYESNQFYNGLIAREKMQFAPIKLSHKVNTKPNRQVADETIYSTRKVDGQDILIEKIKNIYDPKMTRLTDDIINQNSQKYLMAKHDPQTFELIEKIIIDHFNTYKDSPKHYKANTKKGIITYQLVGENPLTAYKNEFGFVCKYSKKGNGPAIISMKYESEQLGNHIDISSNYNVSDKKVILKQLSPYRTDFYQCSDGKYRFVTIYYKDVHYQKSIGKYIIDKTWYQKEMDKKKIDDKAQFLFSLHRDELLGVVKNDGEKYIYDLSKENDGDIRYHDGQNVEILKFTATNNDKKGIVEVKPIYTYCKKQLMISPLNFKIISKYATDVLGNLYEVRQNILKLEFD

SEQ ID NO: 26 - protein, 1314 aa, synthetic construct
MEYNLGLDIGVGSVGWCVTDINGNIIKKNNKNLWGSRLFNEAQTAKQRRTYRGSRRRLERRKERINMLQSLLLDDMEKQYPNFFPMLRDTSLFIEDRKVNKDKYNLFNDTSFSDPIYYDKYPTIYHLRKHLVESKEKEDIRLVYLAIHHIIKYRGNFLYEGDLNENFDNTKLSILVDFLKENDIDFNNNEEDIKKIITNVADNKATKKELFKSLFHTQDTITKNILDNVSKALFGYKFDLNKIFKSDYEIKSISFREEIPDEEDIIEMLGDDAELYVILKDIYNYMVLYDIKKDFKYVSDAFIEKYNQFKIDLKLLKSIYRKYYASEYSDMFRKKKENNYWHYNKEILVKSDKKQSKFSEEEFFKTIKKKIENLPDSFNEKENILSRINEGDFLKKLNTTDNAAIPYQLNKIELEEILKNQSQYYETLSKNNEKIISLLIFKIPYYAGPLSQGDSSKWGWVIRNSDEKIYPWNYDKVVNVDKTAEEFITRMTNNCTYLVNESVIPKNSLLYSEFCVLNELNNIRINGYPIAKDFKIKIVKQIFKNKNSVSEKDIRKFYKENGITVTSFTGLSKDDGKFISNLKSYRDFEKIFTNINDNNKDLCEEIIYWITIFEDKDILKQKLENINLNQQQIKAILSLNYSGWSRLSKKLINELKSNTGETIMDILSNTSMNFMQIITDEELGFDKQIDAMLPKGSNKLSYTELIDELPTSPANKRAIWQTLKVVKELKKVMKGSPSNIFIEFARREDEKIRTTDKLKTLKKIYDEIDKYNRDENVYNELKAHINDKNISEKLYLYFMQHGKCLYSGTPLSINELNLYEVDHILPQSYTSNDSIDNKALVIKRENQRKKDSLLLSDTIINKQINWWKNLLDNGLISSIKYANLTRREIIKTENDGEKFVKRQLVETRQITKSVANILKNNFENTNVLTIRAYLVTNVRKKYDLYKNRNVNDYHHAYDAFILCQVGNIINNKFKHKEKFVYGDYVKKYIEEEYEKYLKTKKLSSDGIVIGLIRKYIDATKLKKVLYYKDCYISRMLEENTGEFYNQTIYSHKFSDNLIPLKKYLPANKYGGFSNLNKAYMAVYSYEEIMKNSIKKRYDLIGIPINISYDIRNGKITLYQYLEETLKDKKIQNINIIIERLLIGQQYSDQNGNTFKLCSEQEIRTDKQLILNELVSKCVSIINKDETKRSQEENGFLKDNYEIVYDYLVEKINNHYTFDTFSSIANKLQIKKNDYNSLESNEKEEVLKEIIKLMSTSQGNLKKLGLGERAGRMSGKSFKDETLLGMTFINKSVTGIYEKRVTINGLENNSNKSSM

SEQ ID NO: 27 - protein, 1355 aa, synthetic construct
MEYYLGLDMGTNSVGWAVTDRQYHILRAKGKDLWGIREFDEACSAEDRRATRTSRRRWQREKARIGLLQDYFKDEIDKVDPSFYQRLDNSFFKIEDKDSEVQYRNILFNDQNFTDADYNRQYPTVFHLRCELIKDPSPHDVRLVYLAALNIFKHRGNFLNPSLSVSGTQIDLTETYSVFRNMLNTYTEIQFPNAFDETLFRSIMCSRRIGRTQRFEALKQFFGVEKAEKQKLEILRALAGLKSSAAKLFSIETEEKLEFCFSDAGYDEKLSELEAAIGDDDSSILGSMKQIFDACAWDTIMDGYQYLSEARIASYDKHKKDLKVLKSVFRKYKTPEDYDRFFRSDADGSYGDYVGSYNSGEKHRRRGSKHTAEDFYSAVKKVLKDCSQDDENVRYILSEIELGTFMPKQLTASNGIIPFQAHYKELKAILENASGYLQFLNNINPESGLTKKEEILRIFSFKIPYYIGPLTEQSKKNGGNGWVIRKEAGPVRPWNLDKKVDIAATSEEFIQRMVRRCTYMAGEKVLPKASLAYERFSLLNELNNIRIDGERLDTSLKQEIYEELFLGGKGVTRKRLASYLAGRGLIENENQISGIDERIHGQLASYCRFYNIFGDELKKDSCREMVEDIIYLSTIFGDEKKFLREKLNKKYGDRLDRNTMKRILALKFTDWGKLSRAFLNLQGVQHGANECMTLSEALWRTPYNIMELINSDEFSFKENLSREINNQLGVLQDVTFDDLDEMYFSAPVKRMVWQTLLVMKELEKVLGAAPSRVFVEFTREHQESKRTVSRKQQFLKLFQGSEFEQEWKDLIERSEENGTIRSKKMYLYLHQKGIDLYTGQPIALEDLFDSGRYDIDHIYPQSLTKDDNLDNNMVLTDKTVNNRKQDNYPLPGEMRRKMQGLWFSLHQSHFINDEKYKRLTGTEPLTDDQLAGFIARQLVETSQGTKAVADILKCVLPADSKVIYSKAHTVSDFRNQYGLVKSRAVNDLHHAHDAYLNIVVGNSYYVKFTDSPYRYIKERYSKGRDRYNLPRLFETDISNGKETAWIGSGKQNGNSENMKPEKGTIVTVRKMLSRPSPLMTRLTFTGHGGLTNQTIYSAEKSKPQIYLPLKGTDARYDVTKYGGVTSASTAYFILVESVKGKKKIRTLEAIPIYVASQVEKDSSVLEKYCKDFLKIEKPVIVLRKIKLQSLVKWNGYFLNINGKYDDSRISVRNATQMLLSQESVKYIKKLEKYSEKRELDTAITEEHNLELYDELTMKHTDGIFSRRPNPIGDVLTKDRPVFEALSVTDQITALLEIIKTTAIVVGGQADLTLIGEKKGSGRMKIPKTISSASEMKLYYQSITGLFCEVVDLLKI

SEQ ID NO: 28 - protein, 1097 aa, synthetic construct
MKIGLDIGIASVGWAVVDDDYKVLEAGSNLFESADASKNVERRSFRQLKRLHRRQQNRISDFNKLWKKQGLEIPQKVCNTQLSLRVKGLTEKLTNDEFYYVLVNMLKHRGISYLDDAIEDEGKSSSKYEMGIKANKEQLDEGKFPCQIQMARLDKYGKYRGEIIDGDMIMSNVYTVSSYEKEIEAIFEKQKQENNCITDNFEKEYISIFNRKRKYYEGPGNELSRTDYGIYTTKIDENTGRYKTELNLFDKLIGKCTIYPDLRRAAGASYTAQEFNLLNDLNNLIINNRKLEEEEKHKIVQTILVSNTVNMRKIIEKVIGEKIEVMSGARIDKDEKEIFHKFEQYNKMRKELNEISVDITSFERDELDKIGEILTLNTDKEAILLAFENEGLNYSEEIKECFVQIRKKNGSLFSKWQSFSLQIMNELIPEMYIQPKNQMELLTDMGVFKTSADVYKNYKYIPKDIITQEIYNPVVGRSIRITIDILNALIKKYGYPEKIVIEMPRDRNSEEEKQRISSEQSKNEKELAKIIKSVHDEYGIEITKKDFYRHKQLGLKLKLWDEQGRRCPYSGKPIAIEDILNDPYKFEIDHIIPRSISFDDSRSNKVLVYGVENQEKGNRTPYMYLTNVKREWDWHSYMSYILELKANKLIDKSKVDKLLFTEDITKISVLKGFIARNLNDTRYASRVVLNTVQSFMKAKEADTKVKVIRGSFTHQMRKAMRLDKNRDESFVHHAVDAMLICYSQMGYDTFYKLQQGFIDFEDEKVTDKSSWDECMNEEVFERYMYTDKWSKIRENISDAEKRVKYWHKVDKKANRGLCNQTIRGTREYNGKIEKINKINIYSRDGYKTFVNMINRGKEEQFLMYKNDRRTFEDILKIYNQYKDAVNAFTEYEKETGDYVRKYAKNHKGCRIENLKYRDGEVGACIDISNKYGCKEGSKKVILESLNPYRMDVYYNTNDKKYYFVGLKMSDLKFSGGKYMLDMNKYNNVLVKEKMLKEGENREDLLRKGYEFRLSFYKNEYIKYDKDGELYIERFLSRTMPKARNYIETKPIEASKFEKQKLVGLSKTEYVSKVRVDILGNQYECKREKFELYIDNTF

SEQ ID NO: 29 - protein, 1099 aa, synthetic construct
MGIRIGVDIGIASVGWAVVDDEYQVLESGSNLFDSADASRNVDRRGFRQIKRLHRRRKNRVHDYNSLWCAYGNKIPDENIVDVLDARIKGLSSELSLLELYCVLRSALLHRGISYMDDALDDASSGKSDYQKGIQINQEKLGQDLFPCQIQKERLEKYGSYRGTITVTENNEKVTLSNIFTTSAYVKEVKSILENQMAYHSFIDSDFVDSYVQILMRKRKYYEGPGNEFSRTDYGIYTTKKDETGQYITEKNIFEKLIGRCSVYPEEIRAAGASYTAQEFNLLNDLNNLVVNERKLTEEERRKIVDVIKNSNSVNMRKIIAKVIGEDIETMSGARIDKNDKEIFHTFEQYRAMKKEFAVNGWNIDDFTRDEQDEIAYILTLNTEKEGIMEAFNTAKINLLPEQKELLVQFRKKRGSFYSKWQSFSLKIMKEIIPDMYITGKNQMQILTDMGVFKTSKEKFVNTNKIPADTLLENIYNPVVCRSIRITVNVLNALIKKYGYPDQIVVEMPRDKNSDEEKARIKDMQKKNEKELAGIIKKIRAEHGITIKDEDFHHHKNLALKLKLWNEQKGKCIYSGRPIEIEQLLKDQWAFEIDHIIPKSISFDDSRMNKVLVYEGENRDKGNKTPFMYLSKLKREWDFEMFKNFVCSMEIPSGKKQKLLFMQDITKVEVLQGFIARNINDTRYASRVVLNALQDYFMAKETNTKVRVIRGSFTSQLRKVLQLEKDREESFSHHAVDAMIMCYSQMGFDVYHKMQMEIVDEETGEIFKVGEFDRVMSNKVYNEMMYQNKLITIRKNIAAAKEKTKYWHKVDKKANRSLGNQTIRGTRNKNGKVIKINKLDLYDISDVKRFKEKIKKGKESSFLMFEKDPKTWEQLMLLMSDYADSPNPLQDYLEETGDYFRKYAKKHNGPKIQCIKYYDGEVKSCLDISHKYGFSKGSKKVILEGINPFRMDVYCNEKKKEYILVGVKYADLKYTQGRYVIDEDAYTQILRNEKMIQNDQVYTDVEENGYEFQMSFYKNEIIQYEKDGEFFTERFSSRTMPNKKNYIETKPVDRPKFGEKKQNLVGLSKTNLIEKIRTDILGNRYKANKEKFSLWVDLY

SEQ ID NO: 30 - protein, 1356 aa, synthetic construct
MTICNDYYLGLDIGTSSVGWAVTDPQYNLIRAKGKDLWGVRLFREAETGEQRRYFRISRRRRQREIARIGLLKEFFADEIIKVDPSFYQRLSESKYHYEDKEVSSKHSLFSDKLFTDKEYYKKYPTIFHLREELIENQEQHDIRLVFLTILNIFKHRGHFLNSSLEVNSNNNFIFAYEELKTLIADRLEIELDSSVEAKEFEKILSAKGFSRTRLAEKAADLLGIKKTKDKQVYEIIKSLCGLSFSVDIIYGEKFESDGQKTPGISFRESDYEEKIVGIKEVIDEDYSEILDLLKRLHDQALLSNILQGKDYLSQARVASYEKHKKDLLVLKRAVKEFCPEQYNILFRSNEEGSYSAYIGSVNSDIKQRRSLKKGKQDDIYSTIKKILVEAPKEHPDINYILNEIEAENFLAKQLTTSNGVIPNQLHLSELHAILENAKNHFPFLQATDDNEITVAEKIMELFKFQIPYYVGPLNGYHKGKGGNAWVVRKRQGQVLPWNFDEMIDSKKSSQEFIERLIRKCTYLKDESVMPKSSLLYEKFMLLNELNNLKIRGVKIDVQLKQKIYCDLFLSGKKVTRKKLTNYLVSEGIILPNEEGAISGVDGDFKNYLASYGKFISVLGDIVRREDTQNRIERIIFWGTVYGGDKSYFKENLCEHYSEELSKEQIKRILGLKFRDWGRLSKEFLEMPGVNKETGEMLPLIQMLWSYNENLMELMGERFTFKEELDKEVGAITKTLTEMEHSDLDDLYLSSPVKRMVWQTLLVVKELEKVMECPPKRIFVEMARGSGEKGKRTSSRKEKLLSLYKSCKEDGRDWASEINTKTDSELRGKKLYLYYIQHGLCMYTGEPIEINQLFDNNLYDIDHIYPRRIVKDDSIENNLVLVKKQTNAHKSDIYPIEDNIYNRQKNFWKSLLSKDMKDGFITREKYNRLINRTPLSDEQLAGFISRQIVETRQGTKMLTQILERVFNESDTVFVKAGNISDFRHDFDLLKVRSVNDFHHAQDAYLSIVVGNVYYIKFTRDPMNFIKNFRGGASNESYNMNKLFSSNIRRGNETAWVADGSIENVNSIAIVKKHVNKNTPIITKMIYEGHGELFNATLYSSKKANQKAYVPLKASDNKLVDVTKYGGYSSVSTAYFFLVEHEVKGQKIRTIEALPLIYKEAIGNDVDNLHNYCVESLGYINPDVRVPKIKMQSLIKRNGFYLNITGKTGDQFIVSNAVPLILAPSWAKYIKKIEKTIETQFLEREVSKEKNNGLYEELESKHLSCIFKTRPNPIGNILKEGKAQFKELDEKSQCLVLAQIMQISQLSNMGANLELIGGSAKSGISLISKKISGVDEFVLVNQSVTGLFRSEIDLLTI

SEQ ID NO: 31 - protein, 1354 aa, synthetic construct
MAQNYYLGLDMGTSSLGWAVTDRDYHLLRAKGKDLWGVRLFPEAESAAGRRTQRVSRRRLQREKARIGFVKDVFADAINEVDPGFYQRLDDSKYFPEDKTEMQRFALFSGNGFTDKEYYDQYKTIFHLRSKLIHSAEPMDVRLVYLAVANIFRHRGHFLNANLTDGQSAELEEVYARLVDATSNLPSTVNIEKLKEILSSNKVTNSLRNEQLMELLQISKKMPEAEMLKLVCGLKGMLSKAFTEVEFDEEHQKYSVSFRDGNYEEKDAEMQTLLDEESYGVVQLLKQMHDWGLLANIMTGEQYLSDARVKSYEKHAYDLKILKDLYKEYGNGQYNKMFRVMEDHNYSAYVGSVNSDKEKGKKRRGAKKDRDGFYKKLKAEMQTMQKKAPDDVRIRYILDEIDKEALLPKQLTSANGVIPYQVHLKELKTILSNAEQYLPFLRDKDETGLTKSEKIQQLFAFQIPYYIGPLYNDGNASHNAWVCRKEAGKVFPWNFDQKIDVKASSEEFIRRLVRKCTYLTSENVLPKSSLLYQKFMVLNELNNLKINGVSIDTELKQEMYRDLFCREKKITSKKIKEYLITRGVCPKKTGAEISGIDGDFRSSLSSYLLFCEILKVSVLTYEQEKMAEQIIFWATVYGDTKSFLKEKIEENYGDKLTAAQIKRICGIKFKDWGRLSKAMLELEGADRETGEVLTIISRMWNENVNLMECLSDKYTYLDTIQDEARAISKSFDEIHYDDLEELYISAPVKRMVWQTILVVKELVKVMGGPPAKIFVEMARDVDGKNDKIRKDSRQKKFADLYKKCKDDGRDWSKEIAETPESKFRSKKLYLYYTQKGRCMYTGEPIDLGNLFNDNLYDIDHIYPRHFVKDDSIEKNLVLVKKQINSHKSDNYPLETEIRKSQFGWWKHLCDNGFITKEKFDRLIRSTQFSDEELAAFISRQIVETRQGTKMVTELFENSFPEAEVIYSKAGVVSDFRHKFNLLKCREINNFHHANDAYLNIVVGNTYNVKFTKHPANFIKDYQRDPEHFKYHMDKLFDYPVSRGSEFAWKTKGGESIKTVRKVMSRYTPLVTRMNYEEHGQLWDQTIYSADDAAKAKGVGYVAVNTSDPKMSDVAKYGGYKKYTGAYFFLVEHTKKEKKVRSLEAMPLYLKDSLDSKEKIEAYCREELGYQEPSVRIEKIKMYSLIKVDGFLLYLTGRTGNQLNVINAVEMKLPNEWNTYVKRLFSSSTVIDSNNDISTSGDLISKEKNISLYSILAEKHCAGIYSKRPNPIGTKLKDWKDKFEELPVDKQVYVLKQIMQLSSNSNQGADLQCLGGAAKTGVSLLNKVISGYKEFVLVSYSPTGIYKSENELLTI

SEQ ID NO: 32 - protein, 1352 aa, synthetic construct
MEYYLGLDMGTNSVGWAVTDPQYRLMRAKGKDLWGARLFQDAKTSATRRTFRTNRRRKQRERARIGFLQECFSDEINAKDAGFFQRLEDSKFFEEDKTEKQPYALFADTGYTDVEYFKDYPTIFHLRKELIQSSEEHDVRLVYLAVLNIFKHRGHFLNQNLSDSGLPDLERIYTELVQNYAESLSLDFSEMSDLDYSAIKDLLSTKKLSNSRRAEELTKQFGINKKSASGEMIKAMCGLSATVVKMFPNETIEEESDVYKMKFSFRDSDLEGKLAQLESALSDEAYECLILIKQIHDWGLLASIMTGNGKNYTYLSEARVDQYNKHHRDLILLKKVVRKYSPDKYNEVFRIMKDNNYSAYIGHVNSRIEKTRRGGTKRQEDFYKTINSLLPNNTEDPDVRQIKDEMERGTFLPKLLTNSNGVIPMQLNKIELSAILHNAENYLSFLKKKDENGLTVSEKIVKMFSFQIPYYVGPLLNTENNNAWVVRKEGGKVYPWNFEQKIDVKKSSEEFIARMVKHCTYLGEETVLPKASLLYEKFMVLNELNNLRINGEKIAPELKQELYKALFLKGKKVKAKQVVEYLQSRGLVEKEREVELTGIDGDFVNTLSSNAKFCAIFGVDQLSAEQEKMAEQIIYWSTIYNDSKKFLQEKIQESYGDILSTSQIKRITGIRFREWGRLSREMVELEGADKENGEIKTIIHRMWDENVNLMELLSNRFTYTQAIENRTRLIDKTLNEIEYEDLDGMYLSAPVRRMIWQTILIMKELYFVLKKAPAKIFVEMAREGNAEKVRKQSRKKKFAELYKNIKDEEINWKELIDSTDEGKFSSKKFYLYLTQCGRCMYTGERIELEDLFKDNLYDIDHIYPRHFVKDDSIENNLVLVKKEKNSHKSDVFPLERSIRSQQASFWKTLCDHKLITKEKYNRLMRTESFSEGEMAQFINRQLVETRQGTKMIAKLMQRTFPDSKIVYAKAENVSLFRQKYNLIKCRRVNDFHHAQDAYLNIVVGNTYSVKFTDDPRRFIQEFKKDPNHNKYHMYKLFDYDVVRGVEKAWIAENGESIGIVRNMMAKNTPLVTQYSYKAHGGFADQTVYSAKKAGAAKDGAYMPLKSKDPRLCDVAKYGGVTKISGSYFFLVEYTEKKKRIRSIEAVPIYLAEELNSTGALKEYAEKTLGYTDPDVRIPCIKINSLLEVNGFFATLTGRTGDRLILPSATQLKMDKSSVDYIRLLENNAGEKDDSRLQYKKVSREANEILYGLFIEKHLYGIYSRKPNAIGQKLKDKESVFKNLTIGEQVEVLLQILQISCRVNTGADLKLLKESSSTGITLISKHISNQQSIFMINQSVTGLYQKKIDLLKI

SEQ ID NO: 33 - protein, 1339 aa, synthetic construct
MEYYLGLDMGTSSVGWAVTDSSYHLLRAKGKDLWGVRLFNEANTAADRRVFRTNRRRLQREKARVGFLRECFADSINQIDPNFYQRLDDSKYFLEDKTLNQPFALFSDKEFTDKDYYQKYPTIFHLRKELIESSEPHDVRLVFLAILNIYKHRGHFLNQNLSDDGIGDLNELYCLLSQDDYFCFPDLKETEEIKSILSSKSISNLQRSEEIVKFLNLDKKSKEAEAVKMICGLTGTISKIFAISEDDEEGQKLKISFRDGTLDEKLNQAETTLSEEDYERLLVFKQIHDFGLLSSIMPSSGSKYLSYARVESYEKHHRDLLLLKKVYKKYMPDKFDEMFRIMRDNNYSAYSGSVNSKIQKERRGGKAKTEDFYKGIITDIAKMPKDEDTEYITNEINKETFLPKQLTNSNGVIPMQVHKMELAAILKNAENYLDFLKVKDETGIAVSQKILDVFSFQIPYYIGPLVNNGDNNAWVVRKEEGKVYPWNFEQKIDVKASSEEFIKRMVNHCTYLTGETVLPKQSLIYQRFMVLNELNNLKINGEDITVELKQDIYNDLFKKGNKVKGSTIIKYLKSRNLIDAKDTPEITGIDGDFVNSLSSLKKFRDVYGVDELSGEQQKMTEQIIYWSTVYGDSRKFLNEKITENFGDILKKEQIKRITSLKFKDWGRLSRQMLTLEGASKGTGEIKSLIGRMWDENLNLMELLSDRFTYLEEINKRVKAINKSLSEITYEDLDELYISAPVRRMTWQTILIIKELYSVLGEAPRKIFVEMARDTDAKKERKDSRKKKFIDLYKSCKDDGVNWSKQIKDTPDERFRSKKLYLYYTQKGRCMYTGEIIELQNLFNDNLYDIDHIYPQSVVKDDSLENNCVLVKKTENAGKSNTFPIAESIRHKQASFWKTLLDGEFITKEKYNRLMRREAFSDDELAQFVNRQIVETRQGTKFIAELFKEVFPDSDTVYVKAGNVSEFRKRFDLLKCRSINDFHHANDAYLNIVVGNVYDVKFTKHPINFIKEYRKDPKSHPYHMNKIFDYSVRRGDQIAWIAENGESIAVVKKMMQKNTPLVTQMNYEVHGGFAEQTIYSADLVNKAGGEGYIPVKCNDKVANINKYGGYSSVKGTYYFLVEFTKGKKRIRKLEFLPLYLKNSLNDIKALEEYCKDKLHYEKPSIRLCRIKMNSLVKRNGYFVTLNGRTGMQIYASNAVQLIMDKKHQDYVHLLDKYSGINDSEYLKRNNISKESNEEIYQYLVEKLNNKIYDNRPASILSKLKSKYNVFKELELSEQNYVLKQIVLYFSRANISIDLKLLNEKSSPMKISNTVSNEKEFILINQSVTGLFKNEINLLTI

SEQ ID NO: 34 - protein, 1092 aa, synthetic construct
MNYNVGLDIGVASVGSAVVTEDYEIVESTSNIFPEADAAENLTRRSMRQNRRLTRRRKTRITDFVKLWKRMGFDVPESFDCNVLELRVRGLTEELSMDELFLVLQNDLKHRGISYLEDATDVSDSSQYAKGLKKNHEEMEDKYPCEIQLERLSKYGFYRGQHTIKESNEEVVLSNVFTVSAYREEIEQLFKTQRKYNEKITETFEEKYLDIFRRKREYYVGPGNEYSRTDYGKYTTRVNPVTGEYITEENIFEKLVGKCSVYTEERRASAASYTAQEFNVLNDLNNLVVNGRKLEKDEKIEIIRLMKTEKTVSVPRFLKKVMGETVESIEGARIDKNDKQIFHSFEIYNKQRKAFDELGMNIDDIFSVDELDLIGDILTLNTDKDSILSALNRNGFLLDEQISDCLISIRKKNTNLFNKWQSFSIKIMRELIPAMYEESKNQMQLLSDIGVFKSNTDKYKGLKYIPDKELLEEIYNPIVRRSVHTSIKVLNAYIKKYGYPQKIVIEMPRDKNSDEEKKFIKKIQDENEKELDNIKKKCKSEYGFEIKDEHFRNHKKLALKLKLWNEQKGKCLYSGRDIDIKELVENSSSFEVDHIIPLSISLDDSRNNKVLVYAIENQRKGNNTPYKYLKGIDRDWDYEKYREYVLSLGLTNSKRKKIQNLLFEQDITKLEVVRGFINRNLNDTAYASKLVLNTLQSFFKANDTNTKVYVIRGAFTHQMRMNLKLDKDREINSHHAVDAMLIAFSKMGYDAYHKLQENVIDFETGEILDGHDENDIVDDATYKKMLYEDKWFEIRTRISKAEKEVKYNYYVDKKINRGLCNQTIRGSREYDGKIYKINKLNIYTQDGIKTFKKKIEGGKEDQFLMKKNDPKTFEDLMSIYNQYKDAANPFVEYEKETGEYPRKYSKKHNGPRITNMKYTDGEVGSCIDISHKYGYEKGAKKVFLESLVPYRMDVYFNKNDNLYYFVGLKHSDFKYIKGKYMLDDDAYNRVLQEEKLIDSTQTHKDLEARGFEYRLSFYKNDLISYEKNGEIYERRFISRTMPQKRNYIEIKSVDGKELDKRTDGLSKAMNVTKIQTDILGNKFYVKREKFLK

SEQ ID NO: 35 - protein, 1089 aa, synthetic construct
MGLRFGFDIGIASVGVAVVDDNYDVKEAISYIFPAAEASENVSRRSFRQMKRLHRRKANRLNDFNKLWIKYGYKIPDIKINEVIELKVRALSQEISMDELYAVLHHSLKMRGISYLEDAIDENGAKGDYQRGIAINQKELEEKEYPCEIQYERLRKYGSYRGENTVEIDGEKVTLSNVFTTGAYKKEIEVILKKQSEFHGEISESFIKDFMDIFSRKREYFVGPGNELSRTDYGKYTTKKDAEGNYITEDNIFEKLIGKCSINPDETRAAAASYSAQEFNLLNDLNNLTVNERKLSEEEKRKIIDIVKNSNSVNMEKIIKKVIGEDIYKWTGFRVDKNDKPEFHTFKQYNLIRKALEEIGVDIQDFDRDTLDNIAYVLTINTDKEAILKGFDRENIELSDEVKEVLISVRKKNSSYFNKWHSFGLTIINELIEPLYKEPKNQMELLTNMGYFKPDVERYKDYNSVPVNELLDNIYNPVVTRAVRITIKALNALIKKYGYPDEVVIEMPRDKNSDEEKKRIDSNNKANAKELDEIVKKIKLEYGIEISESTFYKHKGLVMKLKLWNEQGGKCLYSGKTIDVKDLINNFDMFQIDHVIPKSISFDDSRNNKVLVYGTENQNKGNMTPFMYLSTVNRDWNYDKYKEYIKSLGYSRNKRKNLFFEEDITKMEVLQGFINRNLNDTRYASKVVLNTLQSFFRAKDVDTKIKVIRGSFTHMMRVNLRLDKDRDESYSHHAVDAMLMCFAQMGYEAYRKLNHDIVDYESGEILDEKELIKSFDDNKFDDLLYQDKWMNIKNGIKKAEQEVKFWFKKDTKVNRGLCNQTIYGTREVDGKTIKVNYLNIYTKDGYKRLLNLIKKGKQEDFLMYRNDPKTWENLMTILEDYKDSANAFVEYEKETGDCLRKYSKKHNGPRIEKIKYLDNEVNSCIDISHKYGFKKNSKKVILESLNPYRTDVYYDLEKKIYYLIGIKYSDIIMQHGEYYIDEKKYNELLIKEGAKLDRINTEIVFKFSLYKNDIIKYEKAGEIYLERFSARIMPNVKGYIETKPVNKSKFEKRNLTGVNKSLFIEKINLDDLGNQYKSVGEKFSLKVDM

SEQ ID NO: 36 - protein, 1350 aa, synthetic construct
MEQKYYLGLDMGTSSLGWAVTDQNYHLLRAKGKDLWGVRLFQEANVSASRRTNRVSRRRLQREKARIGFVRAVFADAINEVDPGFYQRLEDSKYYPEDKAEHQKYALFTGERFTDKEYYEKYPTIFHLRSDLLKASEPRDVRLVYLAVLNIFKHRGHFLNANLSDEKTNGFEHIYAQIAERIEKFPSNPKFEYLKEILSSNKGTKSQKAEQMIAALHFNKKDPEAEMAKMVCGLKAVLPKAFPDCYFDEEHTKYSVSFRDGNYEEKETEMQEFLDEETFEIVQLLKQMHDWGLLANIMNGEEYLSDARVASYEKHASDLKILKDLYKQYGCGSYNKMFRVMEDHNYSAYVGSVNSDKERGKERRGAKGNRDDFYKRIKNDLKKMLEKASDDARISYVLDEIEKETFLPKQLTFSNGVIPYQVHFRELKQILENAETYLPFLKEVDETGLSNTEKILRLFSFQIPYYIGPLYNDGNPAHNAWVVRKSRGKVFPWNFEEKVDVKASSEAFIQKMVRDCTYLQGETGLPKESLLYEKYMVLNELNNLKVNGVPIDVELKQNIYLDLFTHHKKVTEKKIRDYLITNGAVAKNIPTELSGIDGDFKSSLRSRIIFQDIFETETLTFEQEQMAEKIVFWSTIYGDSKPFLKERIEEHFGGKLSEKQIKRICGLRFRDWGRLSKAFLQLEGADIETGEIKTIIERMWDENLNLMECLSGRFTYIQEIREQGASINKSFHEFRYEDLEDLYVSAPVRRMIWQTILVVREITKVMGCPPDKIFVEMARDPGDKNEKVRKDSRKKKLFDLYKNCKGERDWCKEIDSIEEARFRSKKLYLYYTQMGRCMYTGEPIDLEDLFNDNLYDIDHIYPRHFVKDDSIEKNLVLVKKQVNSHKSDTFPIEKEIRVAQSNWWRQLCDLQLITKEKYERLMRNTEFSEAEMAAFISRQIVETRQGTKVITTLFENSFPETDVIYTKASTVSDFRHKYGLLKCREVNNLHHANDAYLNIVVGNVYNTKFTNNPINFIKEYQRDPEKYKYHMDRLFDFTVVRGDQIAWVAKGGESIRTVRTTMAKNTPLVTRMNYEAHGQLWDVQICSAKDAEKAKGVGYIPIKTTDRKLHDVMRYGGYRKYTGGYFFLVEHVVKGNTIRTIETMPIYLKRNLDTKEKMETYCRDVLNLEEPHIRVERIKMYSLIKVNGFPMYLTGRTGNRLVVVNAAELKLSKEWMTYIKRIFSSADEGEQAEGGLNYITRDKNVELYDILMVKHCKGIYSKRPNPVGAKLSEWRITFQKLSVDRQIYVLKQILMLSSNTNQGSDLHDLGGASKTGVSLLSKKVNDYQEFKLISQSPTGIFETERDLLTV

SEQ ID NO: 37 - protein, 1370 aa, synthetic construct
MARKYYLGLDMGTSSLGWAVTDPEYHLIRAKGKDLWGVRLFQEAESAAGRRTQRVSRRRLQREKARIGLVKEIFADAIDKVDPGFYQRLDDSKYFPEDKTEEQRFALFAGDSYSDKDYYKEYPTVFHLRSDLIHSADPKDVRLVYLAILNIFKHRGHFLNANLSDGDADGLEEIYAQLVERTVNLPATYDAGKMKDILSSRKVGNSRRSEQLMELLQINKKMPEAEMVKLICGLKGVLPKVFPDIEFDEENKKFSVSFREGNYEEKDAEIQSMLDEDSYETVQLLKQMHDWGLLAHIMGGEQYLSDARIQSYNKHADDLRLLKELYREYGKGQYNKMFRIMADNNYSAYVGSVNSDKEKGKVRRGAKKNRDEFYSRIKKDLKAFQEKVPQDERIQYVLDEIDKESFLPKQLTSANGVIPNQVHLKELRKILSNAEGYLPFLRNTDESGLTNSEKIVALFSFQIPYYIGPLYNDGAHNAWVVRREAGRVLPWNFNKKIDVKASSEEFIQKMVRHCTYIQSEYVLPKNSLLYEKFMVLNELNNLKINGNPIGVELKQSIYNDLFRREKKVTDKKIREYLIVRGLASKKDEIEITGIDGDFKNSLSSYLRFREIFSTDVLSYEQEKMAEKIIFWATVYGDSKSFLKERIKEKFGDQITDKQIKRICGIKFRDWGRLSKAVLELEGADRETGEVLTLIGRMWNENVNLMECLSGHYTYIDELQEKGREINKSFEEIEYEDLEDLYISAPVRRMVWQTILVVREIVKVMKCPPAKIFIEMARDAEGKNDKTRKDSRKKKFEELYKGCKEDGREWSKEISDTPDSRFRSKKLYLYYTQKGRCMYTGEPIELGDLFNDNLYDIDHIYPRHFVKDDSIEKNLVLVKKQLNSHKSDTFPIEAEIRRVRFAWWKNLCDQGFITKEKFERLVRSTQFSEEELASFISRQIVETRQGTKTVTDIFENSFPDSEIIYSKAGVVSDFRHRFDLIKCREVNNFHHANDAYLNIVVGNAYNVKFTKNPINFVKQYKRDPEHYKYHMDKLFDYPVIRGDEVAWKTKGWESIRTVRSVMARYTPLVTRMNYEEHGQLWDINICSADDATKAKGVGYVPVSSTDGRMHDVTRYGGYKKYTGAYFFLVEHTVKKQRIRTLEAMPLYLKDKLNSKEKMEQYCKDTLGYQDPSVRLKKIKMYSLIKVNGFLLYLTGRTNKQLTVTSAVETKLQAEMNEYVSRLIKCTEDPKNVSGAVEKWNELVNKNRSEGKNNRNTRSLINKENNKTLYDALKEKHTNGIYKNRPNSIGDKLAKYEEEFENITIAEQLLVLKQILQLTSGSKKQADLSLIGGSPNEGVPTLNKKINGYNQFMLVSMSPSGIYEYETDMLTI

SEQ ID NO: 38 - protein, 1359 aa, synthetic construct
MEKRDYYLGLDLGTDSVGWAVTDTNYNLLRAKGKDLWGIREFNEALVAADRRSHRVNRRRRQRELVRIGLLKGYFSDALQSVDPLFLIRLQESMFHKEDKDRALSGNVNGIFNDKDYTDKDYYKEYPTIFHLRKELIDNKSPHDVRLVFLAILNLFKHRGHFLNATLSSEGEIRPFISVYKELLVNLQENFEINLPESINEDEILHVLGDRSLSRTRKKEQISNALSIDKKEKSKDAIIKLFCGLAVKASALIGDSFDSEEDINICFSDAGYDEKSEEIYSKVGEEWSNVIELCKEIYDIGSLSEILRGNNYLSFARVEDYNKHKKDLEILKKLFHQTSEEAFDHMFRSNNPGTYSAYVNSTNSNKKTRRNVKERKRDDLYKTIKSYAKGWNPEDPDVKYVLAEIESENFLPKQLTFANGVIPNQVHLKELKAILRNAEGYLPFLLEKDEYGNTVSNRIEQLFSFQIPYYIGPTSEKSKEMNGNGWVVRKEAGPVLPWNIDEKIDIKATSEAFIEKMVKKCSYISDEKAMPKNSLLYEKYRVLNEINNIRINDHRITTELKQDIFNDLFLSGKRVTRKKLESYLIGRGAIENAAQLSGIDININNYLSSYGKFKDIFGESIKEDSTKNMAEDIIYWCTIYGDSKKFLKERISEKYKDKLSEKQIKSIMGIKFSDWGRLSKTFLELPGTDKETGEKITLIRAMWDNNLNMMELINDTEHFTFKESLEDMVISGTKVLSEFSHEDLNDYYFSAPVKRMIWQTLSIIKELESVLGAPPKRIFIEMTRKPDDKKERKESRGKQLIDLYKNIKAEDKAWKNEMISKIESADSDGSLRSKKLYLYYTQMGRDMYTGEVIDLDELFNNNLYDIDHIYPQQLVKDDNLSNNLVLVKKQKNSEKQNVYPLDEKIRKSPKVRDLWNMLHLNNLINDEKYKRLTRNTGFDDDELAGFIARQIVETGQATKGVADLLKTLLPEPETKVVYAKASAVSEFRKDNGFLKSRLINDNHHAQDAYLNIVVGNAYYVKFTQNPINYIKKEYKDKKDSYHLGKMFKNTITRGKETAWIAAGEGEEGTIKTVKKVMSRNTPLLTRLSFVGHGQISEATLYGADTTKEKGYLPLKEKDDRFLNMAKYGGFSGISVAYFFLVEHISKKKPVRTLETVPIYIADRIESDPSALKRYCIEKLKLVDPNIRLRKIKLHSLVEKDGYFGHITGVTGNRIYFRNAVSLILPIEWNNYIKTIENSIEKKSLNDIITANKNISLYDLLTNKLSNSIFAKRPNPIGDNLQVSRDRFITLNLEEQITLLEQILHLTAIGMAKADLTSLGYSKLSGISLLNKEITKSNSFTLVYQSITGLYEKRIDLLTV

SEQ ID NO: 39 - protein, 1097 aa, synthetic construct
MGKIIGLDIGIASVGWAVLDKDSYKIVEAGSNIFPAAEASKNQERREFRGLRRMTRRRRTRLDDFKKLWKNYYKNEYSVTPNNVTELKVKGLSEQLSELELYTVLYSYLKHRGISYLEDAVDEGKTGSDYLKGLQTNQKEMNTKFPSQIQYERLEKYGCFRGENKVSDDEVLSNVFTTSAYKKEIEELISVQDNLGNELDKDFIKKYFEIFSRKRKYYEGPGNELSRTDYGIYTTKIDENGNYITDKNLFEKLIGKCSVYPEEMRASAASYTAQEFNALNDLNNLIVNGRKLTKEEKISIIECYKTEKTINVRKIIKNVIGENIDQLSGYRMDKNDKELYHTFEVYNKLRKAFEEAGISFDFSREDFDEIGRILTLNIEREGIEDAIRISNLVLSSDAVDVLISVRKKNASLFSKWHSFSLKIMNELIPEMYEQPKNQMELLSDMKSIKSKMDEFKGLEDIPVETIIEDIYNPVVKRSIRITIKIINALKKKYSDIEKIVIEMPRDKNSDEEKERIKKFQKDQENELPNIIKRVKDEYGITLTPELIARKKGLAFKLKLWNEQNGRCPYSGRMIDIESLINDGDLFEVDHIIPLSISFDDSRSNKTLVYRVENQEKKNNTPFMYLSRLNRQWNYDKFAAFIKELYGSSGKDRKKFRKKMNNFLFTEDINKIDVLKGFINRNINDTRYASRVVLNSLQGYFRANDNNVKVSVINGSFTHQMRVNLSLGKDRDESFAHHAVDAMLIALSQMGYDAFRARQKDIVDFETGEIINGSAWENQDEMYKTVLYQEKWKNIKDNIIEAEPNIKYWHKVDKKCNRGLCNQTIYSTRKCDDKIWKVDKLNIRDKDDVKKLIYMIQNGKEDKILMAQNDPKTFANLKYILDEYSDCENPFVSYEKETGDLVRKYSKKHNGPKITYLKYMVEEVNSCIDISHKYGHEKGSKKVFLAQLVPFRTDVYYNNNSGKYYLIGIKQSDIKCKGNQYIIDEEKYTDNLIQNGLLKEGQKIEDLEKLGYLFKFSIFKNDTIEFEKKGELIKCRFLSQNSASKNRIEVKPIGKKDYESRNLPTLSTTTVINKYVKDILGNEKQVSGEKFEIIVDSK

SEQ ID NO: 40 - protein, 1351 aa, synthetic construct
MKYYLGLDMGTDSVGWAVTDEHYNIVRAKGKDLWGIREFEEAKTAADRRIQRTSRRRLEREKARIGLLKDYFKDEIDKVDPSFYARLDNSFLKIEDKAEEVQYKNILFNDEDFTDRDYYKKYPTIFHLISELIHNTTPHDVRLVYLACLNIFKHRGNFLNPALSKDNTRIDISDKYTELRQALSEQTNIILSEEFNENSFKAIMCDSSKSRTLKYEGLKDLFSIEKSDKQKQEILKAIAGLKVNAKNIFDLDTEEKIEFSFYEGNFDDKAAELEKEMGEDFFNIIALMKEIYDACEWDTVMAGYEYLSDSRVASYEKHKKDLVILKRAFRNNKSKDDYERFFRTDEDGTYGDYTGSNNSREKHRRRGSKHRQEDFYDSVKKEFKGLEKNGDPDIDYILTEISNGTFMPKQLTPSNGVIPFQAHYKELNAILNNASNYLGFLNDIDSESGQSVKADILQIFEFKLPYYIGPVTTKSAENGGNGWVIRRENGKVLPWNIEQKIDTKATSEAFIQRMVRKCTFLSDEKVLPKSSLLYEKFSVLNAINAISIKGERISTNIKQDLYHDKFENGKKVTKTQIVRYLNGRGLDVTENDITGIDDIITSQLSTYHRFYQIFGDDMKKDSYKNMAEDIIYLSTIFGDEKKFLKDKIKEKYGDKLSDEQIKKVLSIKCTDWGKLSKEFLELKGCRKNTGEEISLIDAMWETSLNLMELINSDEFNFNEVLNERQTKAFSSLKELKPEDLDDMYFSAPVKRMVWQTLLIIKELEQVIGSAPERVFVEVTRSHEESKRTVSREKLLLDLYKNEDKYWKDIISEASVNGRIRSKRMYLYLKQQGKDLYTGKEIPLDEVFNKGKYDIDHIYPQSLTKDDSLDNNMVLTDKSVNNHKQNIYPLEKSIRDNCYALWTALHAKGFINDEKYKRLTGNQPLTDGQLEGFIARQLVETSQGTKAICDILTQALPATKVIYSKAKVVSDFRNQYHIYKSRIANDLHHAHDAYLNVVVGNSYYVKFTDSPHRFIKERFRCDGGKYNYNLATFFESDVSNGKETAWIGSGKEYNNDNEKGTILTVRKMLAKTSPMMTRLNFVNKGSIAKEQLISADKCKKDSYLQIKVNDARLDVTKYGGYQKVSAAYFFLVESAGKKGEKIRTLETVPVYLADKIEKNPALLEQYCKDILKLNNIRISVRKIKIQSLIKRNGYFLHISGKSSNQILLRNATNIFYNQEKINYIKKIEKYVDKQEVDEEINTEKNIELYDYLTAKHNGKILLHRPNPIGEKLTKRKDKFAELSLAEQILCLYEILTSTAIVAGGAANLKQLGESPSTGVMTMNKNISKEKELLLINQSVTGIYTNTIDLLKI

SEQ ID NO: 41 - protein, 1347 aa, synthetic construct
MEEKEYYLGLDMGTNSVGWAVTDPQYIILRAKGKDMWGIREFESAETAETRRVHRTSRRRRQREVVRMGLVKDYFSDAINNVDGNFYARLENSKYHLEDKDETVRNKNGIFADEHYTDKEYYAEYPTIYHLIMELINNPAPHDVRLVYLAVANLFKRRGHFLNAGLSEDGGNQSMQTAYDEFNTTCNETLEFSFPGIDATQIEEVLKNRNYSKTVKSEKLAELLGVEKKEKQRIEILRAIAGRKVNAKVIFPNVETEDKVEFSFSDAAFDDNATELMTLLGDECYSLVLCMREMYDIGTLAEIMHGYQYLSESRMADYQKHHEDLVLLKSVIKSECTQKQYDELFRLDGEGSYSAYVNSILAKNHKQRRSMKGRTRDDFYKKVREFIGKSSNPDAIKICTEIENETFMPKMLTSANGIIPNQVHYKELKAILSNAEKYLPFLLEKDESGLTTSDRIQRLYKFRIPYYVGPVSNRDGKGNGWAVRKEEGMVLPWNLEEKIDLSKTKEAFIENLIRRCTYISDEKVLPKNSLIYEKFTVLNEINNIRINDERISVELKQDIYNDLFKKGKKVTRKTLAKYLINRGAIKAENQISGIDIAINNSLSSYGKFKAVFGESMDTDSTKKMVEDIIKTSTIYGDDKRELKNILRDKYGEILSEDQIKRIAGFRCKDWGRLSRAFLELAGTNCGTGEVTSIIQALWESQLNLMELLHSEDYTFGESLNEKLVKNEKSLSEITAEDLDDYYFSAPVKRMAWQTILLIKEISKIKGCEPSKIFIEMTRTDEEKGDKGRKNSRAKELTALYKNIKKEGKAWIELISKEDASGRLKSKKMYLYIKQMGRDMYTGEQIDLDRLFDDNLYDIDHIYPRHFVKDNNIENNLVLVNKAKNSRKSDNYPLDSDIVRNPKVRELWEMLHTNHLMSDEKYHRLTSRKAFTDEDMAGFIARQLVETGQATKGVADLLKDLTNSEIIYSKAGNVSDFRHTYDIPKSRIVNEFHHAHDAYLNIVVGNAYYTKFTSNPLNYIRKEYKKNTKENNYHLNKIFASTISRNGYVAWFAGSDKNPGTIQTVKKMLSRNTPLMTRMTFVVHGSIAEETLYGKNTAKEDSYLPLKGSDPRMSVEKYGGFKKASGAYFFLVEHELKGKKVRTIETVPVYLMKKLSDKRELQKYCEEKLGLINPSIRLKQIKMQSLIKLNGYFVNVTGRTNKQLTLRNAVNLKLDKEDIYIIHLVEKCIGEERDDRLSEESILSLYQTLTEKQVSGIYRNRPNSVYEKLNEGMIRFKKLERKQQIKVLANMLNLTMVGKAQADLKDIGGSANSGVMLMAKKINNYNEVKLINQSVTGLYQTEVDLLTV

SEQ ID NO: 42 - protein, 1348 aa, synthetic construct
MEEKEYYLGLDMGTNSVGWAVTDPQYRILRAKGKDMWGIREFESAETAEKRRGHRTSRRRRQREVVRMGLVKDYFSDAINIVDGNFYARLENSKYHLEDKDEAVRNKNGIFADEHYTDKEYYAEYPTIYHLIMELINNPAPHDVRLVYLAVANLFKRRGHFLNAGLSEDGGNQSMQTAYVEFNTTCNETLGFSFPGIDATQIEEVLKNRNYSKTVKSEKLAELLGVEKKEKQRIEILRAIAGRKVNAKVIFPNVETEDKVEFSFSDAAFDDNATELMTLLGDECYSLVLCMREIYDIGTLAEIMHGYQYLSESRMADYQKHHEDLVLLKSVIKSECTQKQYDELFRLDGEGSYSAYVNSTLAKNHKQRRSMKGRTRDDFYKKVKGLIGKSSNPDAIKICTEIENETFMPKMLTSANGIIPNQVHYKELKAILSNAEKYLPFLLEKDESGLTTSDRIQRLYKFRIPYYVGPVSNRDGKGNGWAVRKEEGMVLPWNLEEKIDLSKTKEAFIENLIRRCTYISDEKVLPKNSLIYEKFTVLNEINNIRINDERISVDLKQDIYNELFKKGKKVTRKTLAKYLINRGVIEAENQISGIDIAINNSLSSYGKFKAVFGESMDTDTTKKMVEDIIKTSTIYGDDKRELKNILRDKYGEILSEDQIKRIAGFRFKDWGRLSRAFLELAGADRRTGEVTSIIQALWESQLNLMELLHSEDYTFGESLNEKLVKNEKSLSEITAEDLDDYYFSAPVKRMAWQTILLIKEISKIKGCEPSKIFIEMTRTDEEKGDKGRKNSRAKELTALYKNIKKEGKAWIELIGKEDASGRLKSKKMYLYIKQMGRDMYTGEQIDLDRLFDDNLYDIDHIYPRHFVKDNNIENNLVLVNKAKNSRKSDNYPLDSDIVRNPKVRELWEMLHTNHLMSDEKYHRLTSRKAFTDEDMAGFIARQLVETGQATKGVADLLKELTSSEIIYSKAGNVSDFRHAYDIPKSRIVNEFHHAHDAYLNIVVGNAYYTKFTSNPLNYIRNEYKKNTKENNYHLNKIFESTISRNGYVAWLAGSDKNPGTIQTVKKMLSRNTPLMTRMTFVVHGSIADETLYGKNTAKEDSYLPLKGSDPRMSVEKYGGFNKASGAYFFLVEHELKGKKVRTIETVPVYLMKKLSDERELQKYCEEKLGLINPSIRLKQIKMQSLIKLNGYFVNVTGRSGNQVKLRNAVNLILSNKQVEYCRLVEKTTSNNSLDERVNAEANLNLYNVLVAKQGQGIYQHRPNSVYQKLKDGREVFTKLSLMEQCSVLMQVLNLTIVGQTLGQLTEIGASSKTGTMKISKRIDLNDTVFLINQSVTGLYQTEVDLLTV

SEQ ID NO: 43 - protein, 1354 aa, synthetic construct
MAQKYYLGLDMGTSSLGWAVTDSEYHLLRAKGKDLWGVRLFPEAEAAAGRRTNRISRRRLQREKARIGFVRDVFADAIGKVDPGFYQRLDDSKYFPEDKTEVQRFALFSGNGFTDREYYDQFKTIFHLRSKLIHSTEPVDVRLVYLAVANIFRHRGHFLNTNLTDGQTAELEDIYAQLVDVTSNLPASVDTEKMKEILSSNKITNSRRNEQLMELLQVSKKMPEAEMLKMICGLKGTLSKAFTDIEFDEDQQKFSISFRDGNYDEKDAEMQTLLDEDSYETVQLLKQIHDWGLLANIMRGEQYLSDARVKSYEKHAQDLKILKELYKEYGSGQYNKMFRIMEDHNYSAYVGSVNSDKEKGKVRRGSKKDRDGFYKKIKSDMLGMQKKAPEDDRIRYILDEIDKESLLPKQLTSANGVIPYQVHLKELRKILSNAEQYLEFLKESDETGLTNSEKILQLFSFQIPYYIGPLYNEGNASHNAWVCRKESGKVFPWNFEQKIDVKSSSEEFIRRLVRKCTYLTSENVLPKSSLLYQKFMVLNELNNLKINGVSIDTELKQDIYRELFCREKKITSKKIKEYLIMHGVCSKKTGVEISGIDGDFKSSLSSYLLFREILNVSVLSYEQEKMAEQIIFWATVYGDTKSFLKEKIEESYGDILSPAQIKRICSIKFRDWGRLSKAVLELEGADCETGEVLTIISRMWNENVNLMECLSDKYTYLEAIRNEGRSISHSFEEIQYEDLEELYISAPVRRMVWQTILVVKELVKVMGGPPEKIFVEMARDVDGKNEKVRKDSRQKKFADLYKKCKDEDRDWSKEIADTPEAKFRSKKLYLYYTQKGRCMYTGEPIDLGDLFNDNLYDIDHIYPRHFVKDDSIEKNLVLVKKQVNSHKSDNFPLEADIRTNQYSWWKYLCDGHFITKEKFERLTRSTPFSAEELAAFISRQIVETRQGTKVVTELFENSFPNAEVVYSKAGVVSDFRHKFNLLKCREINNFHHANDAYLNIVVGNTYNVKFTKHPANFIKEYQRDPKHFEYHMDKLFDYPVTRGNEVAWKTKGGESIKTVRKVMSRYTPLVTRMNYEEHGQLWDQTIYSADDAAKAKGVGYVPVNTNDPKMQDVTKYGGYKKYTGAYFFLVEHLKKGKKVRTLEAMPLYLKDSLDSKEKMEEYCREKLGYQDPSVRVERIKMYSLIKVNGFFMYLTGRTGNQLNVINSVEMKLPAHWSAYIKRLFAYSPSSDSDIEESDVKDLVTNERNCTLYSILAEKHYSGIYSKRPNPVGTKLKSWEKSFEALPIDKQVYILKQLLQLSSNSNQGADLQYLGGAAKTGVALLNKMINGYREFRLVSLSPTGVYKSETDLLTI

SEQ ID NO: 44 - protein, 1348 aa, synthetic construct
MERNYYLGLDMGTSSLGWAVTDPEYHLLRAKGKDLWGVRLFPEAESAAGRRTQRVSRRRIQRERARINFIREVFSDAINEIDPGFYQRLDDSKYFPEDKTEKQPFALFAGGMFTDKEYYQRYKTIFHLRSDLLHSGEARDVRLVYLAVLNIFKHRGHFLNANLTDSGTLEIGEIYAQLLEKTVNLPSDIDLDALKMILSSGKITNSRRNQQLMELLQINKKMPEAEMLKLICGLKGTLSKAFPEVEFEEEYQKYSVSFRDGNYEEKETEMMSFLDEDCYETVQILKQIHDWGLLANIMQGEQYLSDARVKAYEKHAQDLNILKSVYKEYGKGRYNKMFRVMEDNNYSAYVGSVNSDKEKGKERRGAKKDRDGFYKKLKVELHKMQETAPDDDRIKYLLDEIDKETLLPKQLTSANGVIPYQVHLKELRKILSNAENYLPFLKEKDETGLSVSEKIAKLFAFQIPYYIGPLYNDGKPGHNAWVCRKEPGKVTPWNFEQKVDVKASSEEFIRRMVKHCTYMQSENVLPKCSLLYEKFMVLNELNKLKINGIPIDVDLKHDIYRDLFCKGKRITARKIRDYLVVRGIVSKKADLELSGIDGDFKSELTSYLRFREIFGVDVLTYEQEQMAEKIIFWSTIYGDARAFLKERLKEHYGDQLTEAQIKRICGIKFRDWGRLSRAFLELEGADRETGEVLTLINRMWQENVNLMECLSDRYTYVDVIREEGNGINKAFAEIRHEDLDELYISAPVRRMVWQTILVVRELVKVMGGPPAKIFVEMARDVNGKNEKERKDSRQKKFLDLYRKCKEEGRDWSKEIADTPESRFRSKKLYLYYTQKGRCMYTGEPIDLGDLFNDNLYDIDHIYPRHFVKDDSIEKNLVLVKKQVNSHKSDSFPIEPEIRKARYIWWKDLCEKGFITKEKFERLTRSTEFTADELAAFISRQIVETRQGTKTITDIFENSFPDAKVIYSKAGVVSDFRHKFDMLKCREINNFHHANDAYLNIVVGNTYNVKFTRNPVNFIKEYQRDPEKYQYHMDKLFDYPVSRGGEIAWKTKGWESIKTVRSVMSRNTPLVTRMNYEEHGQLWDQTIYSADDAAKAKGVGYVPINTSDPKMRDVSKYGGYKKFTGAYFFLVEHTVKKDRIRSLEAMPLYLKDVLDTNDKIEEYCREKLGYIDPSVRMKKIKMYSLVKVNGAFFYLTGRTGNQLIIANAMELKLPYKWCKYIKRMLTFAYEKDDEDSTISRETNIELFDILNKKHKNTVFGKRPNSIGKKLSESVDKFKELNINEQVYVLKQILQISSFGNQGADLQLLGGAAKTGISLVSKKINSQKQFQLISISPTGLYRKEIDLLQV

SEQ ID NO: 45 - protein, 1357 aa, synthetic construct
MMSKYYLGLDMGTATVGWAVTDENYELLRKKGKDMWGVRLFREAETAVARRTLRVSRRRLQRKIARIGYLKEIFAEEIGKVDAGFYQRLEDSKYYGEDKKENQPYALFTNRGYTDREYHKEYPTIFHLRKELIESKEPHDVRLVFLSLLNMYKHRGHFLNTNLGDSGIGSFKDLYFMWQTTAAELMDVNIPKIEDSKWLEEILASRKYSNSGKLYEIIKKFELVKSVEKQQIEMWKLICGLTSTIATIFQNEQLEEEQKKFSISFRDGNYEEKATQVETMLGEESFELFLLLKQLHDWGVLANIMKGEDKTYEYLSFARVDSYDKHGKDLDILQGLYKTYAKKSQYDEMFRIMKDHNYSAYVGSVNSGKEIIRRGGKCNAEEFFKGLKKQVESFPEVPDRDYVLKEIEKGSFLPKQLTAANGVIPNQIHKNEMKKILENAENYLPFLGEKDETGLTYSEKLVRVFEFQIPYYIGPLANGGKNEDSKNHWSVRKEPGQIFPWNFEDKINVKESAEKFIEKMVNHCTYISGEMVLPKNSLLYEKFTLLNELNNLRINGDKISSELKQRIYCELFQGGRKITGKKLEQYLKINGHIDARQEVVISGIDQDFVNRRANYSKFQSVFETEILTYEQENIAEKIIFWSTIYGDTKKFLREKIQEKYGQVLSETQIKKIIGFKFRDWGRLSRELLELEAADKETGEISSVIGRMWNENYNLMEIIASDKFTYREEIVKKQKTIEKTLSEVEYEDLEELYCSSPVRRMVWQTLLILKEIQEVMEDEPSKIFVEMARDPNAEKKRTVSRKRKFAELYKNCKEDGINWSEEIANREEAAFRSKKLYLYYTQKGRCMYTGERIELYDLMNDNKYDLDHIYPRHFVKDDSIENNLVLVKKEKNAHKSDVFPIESQIRREQYRLWKSLREGGFITEEKYKRLNRNDEFSDDERVGFISRQLVETRQGTKLITDLFVRTFPESEVVYVKAGNVSAFRQQHKFVKCRNVNDFHHAQDAYLNIVVGNVYHVKFTKDAKRFIKEYRKEPEKNAYHMYKMFDYDVTRAGQTAWRAKSGESIRMVKSIMRKNTPLVTRMNFEVHGQFSDQNIIGTDQIIKANGKGYLPIKTTDGRLLDVTKYGGFNKMTGAYFFLVEHKQKKELVRTVEAFPLYLKGKLDTKEKLESYCREVLELNEPCIKLEKIKMYSLLKVNGFYLYLTGRTGERLLVCNAVQLVLPEKQLNYIKKITKEGIYSEEDYTRLEVTKQQNQELYNELEQKHCKLIYAKRPNPIGEKLTKGKAIFEKLPLDRQIYVLLQILQLSQLINQGADLRDIDEAKGTGVTLIAKRISSNQEIKLINQSVAGLFENEIDLLSV

SEQ ID NO: 46 - protein, 1103 aa, synthetic construct
MSRLVFAYDLGIGQTGWAVVDADSRDIIESGVNNFPSGDAAENQERRGFRQMRRMHRRKKTRMADFEKLWVSSGFSVPTLSYTDVLSLRNRGLSENLTEDEIFYVLRFMLKCRGISYLEDELNEDATGEYERAIALNQKELQNKLPCQIQKERMDKYGAYRGEREVEIEGEKVALRNIFTTDAYKKEIELFLQVQARGNKKITDSFVKSYQEIFSRKREYYVGPGNELSRTDYGIYTTIKNPDGTFHTDENLFDKLIGKCSVYPEERRAAAASYTAQEFNALNDLNDLVINGRKLDEEEKRLIIEEYKSSGVVNVNRILKKAIGENIDSITGFRVDKDGKELFHTMEIYRKMVKFFVEKGMDITTFSIDELDRIGEILTINTEKDGIIKAFQNSFVKCDANIVDAFVEFRRKNVRMFTKWHSFSVKIMSELIPEMYAQPKNQMVLLTDMGVFRSRSERFIECKEIPIRETTEEIYNPVVRRSVSMAYRITNALIEKYGYPYKVVIEMPRETNSDEEKKKIGEIQRKNEKELSTIIKKIRDDYGFEIKDEDFRNHKGLVLKLKLWNEQGGKCLYSGKEIPIDQLVKNQGLFEVDHIIPISISFNDSRSNKVLVYASENQKKGNRTPLSYLSGVNREWNDEVFTAYVKSLGYGKDKQDYSGKEKLKNLLFAEDITKQEVVQEFVNRNLNDTRYASREVLNTLKDYFKAKEAGTIVSVIRGAFTHQMRVNLELSKDRDESYRHHAVDAMLIAFSQLGYDAYLARQKQIIDFETGEILDKASWEKQDSLYRKLVYQETWYQTRIKINEAEKQVKFYHKVDRKCNRSLCKQTIYGTREYEGDKYVIDKLDLYVNSDVDRFRKMISDGKEKDFLMYENDPKTFENLMTVFHQYETEKNPFVAYNKETGDFVRKYAKNHNGPKIEKIKFRKEKLGSCIDISHKYGLEKGSGRVALRSLKPYRMDVYRNKNTGLYRFVGLKQSDIKCQGNNYVIDEEAYAKILLSEKMIESGQTRTDLGDRGWEFQLSFYRNDIIQYEKKGVIYTERFLSRTMPEDCNYIETKPIDAPDFKKRNLVGLSQTKSVEKIITSILGNQRNVKREKFSEIVDTIR

SEQ ID NO: 47 - protein, 1123 aa, synthetic construct
MGGITMDNKYVLGLDIGISSCGYGVIDLETGKFVDYGVRLFKEGTAEDNGKRRDARSRRRLTSRRHTRIMDMQKLLKENGIMSNDYHPLQNVYELRCKGLNEKLTNDELTAAILHITKHRGSVIETVEEDVKKSDDELSLKATLQHNEQLIKQGKYICQIQLDNLNNKDHIRGHENNFSTKDYVNELNEILHHQELDEQLINKIIDIVARRRAYYEGPGSEKSPTPYGRFIEVDGQIKEIDLIEKMRGKCSVFPDELRAPKMTVTADLFNFLNDMNNLTVDGNKLCPEEKKGILTIVSQKGNITLRQLAKELNVDEVDIKGYRIDKNQKAIFTEFKGYKKIKSILEKEGYSISLNDYEMLDRIIEILTNKKGIQERKDYLYHLEYRLSDSVVDTLANTTGITGYHSLSFKALNLLNKELFESEMNQMQLLHELKLFDKNRVSHKGKKNIESDPEAILSPVAKRAQNETFKVINALRKKYGEFDSIVVEMTRDKNSDEKKKRISNYQKNRENGSKEMDKFLNEKGYDPTKINGKTRMKISLYLQQGGKSAYTFEPLDLNRVIKDPKYTEIDHIIPISISLDDSQNNKALALHSENQVKGNLTPFMAYAANKFNGIGCSYPEFKTNVLSNKNIPYKKKLNLLNEENITKEEVAKKFINRNLVDTSYACRVVLNTLSDYFKDNEIDTKVHTINGRVTDLFRKKIHLEKDRDENYLHHAVDALIVASVKKMNLLNGYLLKQEHDFNNLYNEETGEIITIPDDKVFYDERYIQYIVNLKTLFEESSKYYDHVIEKENMCFNPIKISHKVNTKPNRQIADETIYSTRNVDGTDYLIEKIPNIYDPKDKKCIRLIQDILNGEDEKYLMERHDPQTFDLIRDIVKYHYEQFKSDSKMYKVTKKKGEEIITLVGENPLTKYKEENGVIHKYSKKGNGPEIVSMKYESEKLGNHLDISSNYQTNNKKVILKQIKPYRTDFYQCEDGKFRFVTVRYTNVSYHKAKGMYVIDEQWYKQEKANKKIKETDKFLFSMHRDELMGIKRKEGQPYYYDSSTENVDGVTCYHDGITTEMLKFTATNNDTTGKIEVNPLYTYCKKQIMVGVANLYGVQKYATDVLGNIYEVKDNKLKLEFK

SEQ ID NO: 48 - protein, 1118 aa, synthetic construct
MEGITMDNKYVLGLDIGISSCGYGVIDLETGKFVDYGVRLFKEGTAEENEKRRGARSRRRLTSRRHTRIMDMQKLLKENGIMSDDYHPLQNVYELRCKGLNEKLTNDELTTAILHITKHRGSVIETVEEDAKKSDDELSLKATLQHNEQLIKQGKFVCQIQLDNLNNKDHIRGHENNFSTKDYVNELNEILHHQELNEQLIQKIIDIVARRRAYYEGPGSEKSPTPYGRFIEVDGQIKEIDLIEKMRGKCSVFPDELRAPKMAVTADLFNFLNDMNNLTVDGNKLCPEEKKGILTIVSQKGNITLRQLAKELNVDEVDIKGYRIDKNQKAIFTEFKGYKKIKSILEKEGYSISLNDYEMLDRIIEILTNKKGIQERKDCLYHLEYKLSDSTVDTLANTTGITGYHSLSFKALNLLNKELFESEMNQMQLLHELKLFDKNRVSHKGKKNIESDPEAILSPVAKRAQNETFKVVNALRKKYGEFDSIVVEMTRDKNSDEKKKRISNYQKNRENGSKEMDKFLNEKGYDPTKINGKTRMKISLYLQQGGKSAYTFEPLDLNRVIKDPKYTEIDHIIPISISLDDSQNNKVLALHSENQVKGNLTPFMAYAANKFNGMGCSYPEFKTNVLSNKNIPYKKKLNLLNEENITKEEVAKKFINRNLVDTSYACRVVLNTLSDYFKDNEIDTKVHTINGRVTDLFRKKIHLEKDRDENYLHHAVDALIVASVKKMNLLNGYLARQEHDFNDLYNEETGEIITIPDDKVFFDERYIQYIVNLKTLYQESNQYYNRMIDKEDMHFNPIKISHKVNTKPNRQIANETIYSTRNVDGTDYLIERIPNIYDSKDKRLVEDILNGNDTKYLMSKHDPQTFDLIRDIVKYHYEQFKSDSKMYKVTKKKDKEIITLVGENPLTKYKEENGSIRKYSKKGNGPEIVTMKFESEVLGNHLDISSNYHTNNKKVILKQIKTYRTDFYKCADGKFRFVTIYFKDVSFNRTKGTYVIDRKWYDGEKEKKRIKSTDKFLFSMHRDELLGVVKEQGQPYFYDVYDEVPDKKILHDGVNPEILLFALTGNDKTNVVNLYPIFAYKKRQQKVLTTAKDIRKYATDVLGNIYEVKDNKLKLEFK

SEQ ID NO: 49 - protein, 1311 aa, synthetic construct
MEQKNCKKVDAYNIGLDIGTNSVGWAVTDLDNNILKRGNKNMWGARIFDEGSTAAQTRILRGTRRRIERRKERINILQSLLLDDMEKEYPNFFPMLRETSKTKEDKNSDSINGKKYNLFSELELSDPIYYQKYPTIYHLRRELMESKEKHDIRLVYLAIHHIIKYRGNFLYEGDLKNNNNEIIESINELLDFIQNTLEISFISEAEKIQEILKDKTKSKGEKKDLIIGLFDYDKDEKSVITGIVNAILGYKFDVDKIFDIEVENSNISFSNEIANEEEIKENLQDHAYAYDVLLKIYNWFILQDILQGNNSISEAFINKYDKYKKDLSNIKSLYRNYLKDEYNNMFRKEKDDSYACFEKHTSACPIENLYKRIKKDLADVPDCKEKEEILKDIEDDAFLVKINTTANSAIPYQLHYQELEKILENQAQYYPTIQENKQHILELMKFRIPYYVGPLAQEGKSRFAWIVRNSNEKILPWTFDDVVNVDETAEKFIKRMTNKCTYLINEDVIPKQSILYSEFCVLNELANIRVNNHKLTPKTKQLIIDKLFKVKKTVKLRDLKQVLTDFQVFPEVTSITGLAEVDKFMSNMSSYIDIENILGCINDENIEMVENIIEWITIFEDKKILKRKIKNTYNLEDKVVSQLIKKNYSGWSRLSKELLVGLKAADDGKSIMEKLRTTKDNFMQIINNDMYGFNKQIECRMKLQEKQITYKDVDEIPTSPANKRAIWQSLKIVEEIKKVMKKDPKNIYIEFAREEQEKNKRKDNRAKALLKIYEKYEEEIKMLKDYNPNVYKELKKKQNEKDFNERLYLYFIQNGRCMYSSRPLNIDTLYLYEVDHILPQSYVKDDSLSNKALVYKEENQRKSGSLLIDEKIINRQETWWKQLRKNGLIDEKKYYNLTRRKMFETDKEKVKFVSRQLIETRQSTKYITNLLVNQYSNTDVFAIRSNLTHNFRKFFEVYKNRNVNDYHHAQDAYIISVIGNVIDQKLQYKDEYKYTEYVKKYIKKSEEEANKVWIIMGMVSNNINKEKVKKALEYKDCFITHKLEEQTGAFYNQTLYSPKDKNIEIPLKEGLDVKKYGGYSGEIKAYFTIYSYIDQKGKMQIEMIGIPVKTKYDIENGKTTLIKYIQSKNCEASKIEIIRPKILKYQEYIDENNEPMMLLSDSEIRTNRQLIVNNEMSKLIYLMNQEKVADDEKEKLQEKMEEIYNYLLGKLKNEYKTFNSIYQKLSSEETKEVFQKLEYKDKVSAINGIIDLMHRGQGNLTKLKQGDRAGRKSGKSFKTEALNKMIFIDKSVTGMYERRYKVNGMENCSSK

SEQ ID NO: 50 - protein, 1104 aa, synthetic construct
MGVIIGLDIGIASVGWAVVDRESYKVIESGSNLFTSAEASQNVARREFRQKKRLLRREYNRIKDFEVLWMKSGYSIPTGSRNDILQLRVKGLQEKLSQDELFLVLRNLLKHRGISYLDEALDGADSGNSDYEKGIVKNEQELKEGKFPCEIQLKRLNVYGKYRGRIMIAKDEERITLSNVYTTSAYQKEAEQILETQRKEGVGITEDVVQQYMMIFSRKRKYYEGPGNEKSRTDYGKYTTRINPETGEYVTEDNIFEKLIGKCSVKPNLMRAAGATYTAQEFNVLNDLNNLMVNGRKLEEMEKIEIVEAMKSATSVTVNVEKIIRQVIGTDILSLTGARLDKDDKPEFHQFELYRKLKREFGKCNIDIDKFSIEQLDKIGNILTLNTEKEGILQGFVRQELILTEDEKECLIQFRKKNGKLFNKWQSFSLEVMQELIPDMYEQPKNQMELLTEKKMFQSNMEKFKDCTRIPSACITEKIYNPVVCRAIRVTISIVNALKKKYKSIDQIIVEMPRDRNEDEQKKRITKAQKTNAKELENIIKKIKGEYGITISNEEFRGHKKLALKLKLWNEQEGKCLYSGKAIEILDLLHNPNMFEIDHIIPLSISFDDSRSNKVLVYGTENQEKGNLTPFMYLQRVNRDWDFDEFMSFVLELKERKSISKAKVDKLLEYRDITKVEVLKGFIARNINDTRYASRVVLNTLQEYFKAKGANTKVKVIRGNFTHQMRNALYLEKNREASYAHHAVDAMLLCYSQMGFDAYHKLQEKFIDFEQEIILDTKVWEQQMDSRTYKEVMYQDKWMTIRKNIVDAEKRIKYWHKVDKKPNRALCNQTIRGTRQIDGKIMKINKVDLYSKNGWKTLKKLLDSGKEERFLMYEKDQRTWTDMLKIIEEYRDAENPFVEYERETGDYLRKYSKKHNGPRIVQLRYTDGEVGSCIDISHKYGFDRGSQKVILEKLNPFRSDVYYNRQEQKYYIVGIKYSDLKFEKGKYVISEEAYNQILVQEKMMSVSDTRLDLERLGYEFCFSLYKNDFIEYEKDGKYFTERFLSRTNPKAKNYIETKPIAAAKYEKQRLVGLSKTKSIKKIYVDILGNRYYCKKEKFIKEVDI

SEQ ID NO: 51 - protein, 1115 aa, synthetic construct
MYSGAGGIFDVKKIGGNVMNYVLGLDIGIASVGWAVIDLETLQVIESCSNIFTAAEAENNKDRRSFRQMRRLKRRLRTRISDFAKMWKSLGFSIPGDIDNMPLLLRVKGISEELSMDEIYTVLLNELKHRGISYLEDFGDNANDGTDYSKSIAINQKELEEKFPCQIQYERYCKYGNYRGQFVADINGEKVTLCNVFTTSSYRKEITSILDTQKKFHSEITDKFIENYMKIFDRKREYYVGPGNDNSRTDYGIYTTKLNDDGTYKTDKNLFEKLIGKCSVYNEELRAAAASYTAQEFNVLNDLNNLIVNGRKLEEDEKRKIVDTLKTSDSINMRKIIKKIIGEDIETLEGARIDKDEKEIFHCFEIYNKLRKELKKIEYDINLLSRDTLDDIGRILTINTERDMIVNSLSELGLDNDVVNCLADVRKKNGKLFSKWHSFSLKIMQELIPAMYEQPKEQMQLLTEMGVFKKKGDLFKDCQYIPVDIAKDDIYNPVVVKSIHIAIRIVNAIIKKYGMPEKVVVEMPRERNTEEQKKRIKDTQRKNEKELEEITKKIYAEYELRIEDKDFRNHKGLALKLKLWNEQGGVCPYSGRKIDIFDLVNHPDMFEVDHIIPLSVSLVDSRINKTLVYKVENQEKGVNTPYMYLSKITRDWGWEEYKNYILGLKIEKAKKANYLFMEDITKYDVLKKFINRNLNDTRYASRVILNTLQDFFSTKQTGTIVKVIRGSFTHQMRVNLHIEKDRDESYSHHAVDAMLLCYSQLGYDAFHKLQGSFIDFETGEILDKRMWNENMQEDVYKDYLYGNKWSQIKRHIEDAEKKVKYWHAVDSKCNRGLCNQTIYGTREIDGKIYKISRTADIRTKSGFESLKKLLAESNGKSKKSDILMYRNDPKTFQILMGIMKQYADATNPFVKYEEETGDVIRKYSKKHNGPKVTKISYMDGEVGSCIDISHKYGYAKDSKKVILESLVPYRMDVYYNITDKLYYFVGIKQSDIKCKGDEEIVNMEAYRKALVSEKMILPEQTIDDLEKLGYEFRLSFYKNDIIKYEKNGELFTERFLSRTMPAVRNYIETKPVDAPKFEKQHLIGLSKTKMVKKIRTDILGNCYECEKEEFSFICK

SEQ ID NO: 52 - protein, 1111 aa, synthetic construct
MGIRFAVDIGVASVGWAVVNDEYVVLESGVNIFPCADASKNAERRNFRQLKRLHRRRKNRVSDFEKLWQEENLEIPKNNQINVLQLRVKGLSEKLTQEELFWVLKNELLHRGISYLDDALEEGAANESDYKKALERNQTELTQNEYPCIIQQKRFESYGEYRGNRSFEKDGEIISLSNVFTTNSYKKEIQAILKRQKEFHLFLTEKFQDKYIEIFSRKRKYYEGPGNEKSRTNYGKYTLRINPKTGEYITEKNIFEKLIGKCTIYKDQVRAAGASYTAQEFNLLNDLNNLTVNNRKLTEEEKRNVVNEVKTAKSVNVKRILKKVIGEDIETLTGLVLDKNDKEKIHSFEQYNLMRRAFEKNGWDIEKYTREELDRIGEILTLNTEKEGIIAAFQNKDDQDDFKMINISDEQIECLIELRRKNSAKFSKWQNFSLKLMKEIIPDLYENSKNQMQILTEKELLKNNKEKYKEYKYLPEEDIIEQIYNPVVVRAIRITIRVVNALLKKYGSPRQIIVEMPRDKNEEEQKKKIKDAQNKNKKELDDIIKQVKEEYGIAITEQHFYHHKKLGLKLKLWKEQNEQCLYSGKKIEIYDLLNDQKKFEIDHIIPRSISFDDSRSNKVLVYSSENQQKGNLTPYLYLRGLKREWNFDRFKAVVVELNKKKSITKNKLDKLLCMENITKQDVLQGFVERNINDTRYASRVVLNALQDYFYSHEANTSVRVVRGSFTHQMRVALELNKDRDESFVHHAVDAMLMCYSQMGYETFHRQQKNLIDIETGEILDVEEWDKLMDDEKCTEIMYQQKRMRIIREIKVAQSRVKYWFKVDKKPNRGLCNQTIYGTRNVDGETYKINKLNLYNAEDVKKIRQMIAENQQSKFLMYDLDRKTWDGLVKILEEYSDSRNPFLDYEKETGDAFRKYAKKNNGPKVVKLKYKKEKVGSCIDISHKYGFEKGSRKVLLDSLTPFRTDVYYQPETKQYFLVGLKHSDFKFEGGHYLIKEEVYNQNLIKEKVIQTGQTWKDLGKMGIEFCFTFYRNDIIQYEKNGEVYTERFISRTKKNLNYIEKKPLKKPDWGKEEKRYRTVGLVKTCSIRKIRTDILGNQYVCEREKFRLEVDC

SEQ ID NO: 53 - protein, 1316 aa, synthetic construct
MEQKNLKKYPEYSIGLDIGTNSVGWAVTDSENNILKHGGKNMWGARLFDAGDTAAQTRTFRGTRRRIERRRERVNILQSLLLDDMEREYPNFFPMLRESSKVEDEKQQEKLDGKKYNLFSELNFSDQDYYAKYPTIYHLRKELTTSKEKHDIRLVYLAIHHIIKYRGNFLYEGDLKSNTDEVLESIEFLVNFIRENFEIEFNSTSEKLRNILKDKETTKAEKKDALMRLFDYSKEDKAIINGIICAILGYKFDINKIFDVELENSNISFSSDIANEEEIKDALQENADVYDVLQKIYNWFVLQDILQGNSSISEAFIKKYEKYKKDLENLKNIYKTYLKTEYNNMFRQEEIGSYAMYDKNISKCPIEELYKKIKKDFASIPDCKEKQEILQEIDNEAFLIKINTTANAAIPFQLHYQELEKILENQAKFYSTINENKENILNLMKFRIPYYVGPLAKNDNSRFAWIVRKSDDAIRPWNFEECVDVDATAEMFIRRMTNKCTYLLSEDVIPKQSILYSEFCVRNELANVRVNNHKLSPQTKDLIIEKLFKMKKTAKAKDLKQILIDNQIFKKISSVTGLAEVDKFMSNMSSYIDMKEIFGIVDENSIEMIENIIEWITIFEDKKILKRKIEREYHLDEKIINRLAKKNYSGWSRLSKELLIGLKSFDDGKNIMEKLQSTKDNFMQIINNDQYGFNKQIEDRMKNNDTKITYEMVDEIPTSPANKRAIWQSVKIVKEITKIMKSEPKNIYVEFAREEQKKKVRKDNRAKALLKIYDTFAEEMKMLRDYNPKVYQELKRKQNDRDFNERLYLYFTQNGRCMYSSRPLNIDTLYLYEVDHILPQSYVKDDSLSNKALVYKEENQRKSGSLLLDEKIINKQELWWKQLHKNGLIDDKKLRNLTRRKMYETNDDKVKFVSRQLIETRQSIKYITNLLVNQYKDSDVFAIRAELTHNFRTFFEVYKNRNVNDYHHAQDAYIISVVGNVIDTKLHFKDEYKYTEYVKNYIKKNEEEQSNKRKVWIIMEMVSNNIDKEKVKKTLNYKDCFVTRKLEEQTGEFYKQTLYGPNDKNVKPVIPLKKGMNVEKYGGYSGENKSYFTIFSYIDKKNKKQVEMIGIPVKTSYDIKNNKITLKEYVESQIDNTTEVKIIKPKILKYQEYLDENNESMVLLSDRELRTNKQLVLPSKVNKLIYLMNKEKATEDEKIEVEMNIEEVYNYLIKKLNDEYKSFSSIYEKAKDDKTKNKFNEMSYDDKVVTINGLLDLMNKGQGNLSKIGLGDRAGRTRKSAFKTKDLENMTFIDKSVTGIYERRYKVNGMENGSSK

SEQ ID NO: 54 - protein, 1356 aa, synthetic construct
MKVIDKYYVGLDLGTSSVGWAVTDDKYQLLRAKGKDLWGVRLFEEADVAAGRRVNRVSRRRRQREVARIGLLKEYFAEELSKVDSNFLERLAESKYHREDKKVATKYGIFADDNYTDEDYFREYPTIFHLRYELIQNPQAHDVRLVYLALLNMFKHRGHFLNAGLKTGGETESMEDIYGLLYMTAQEWEIELPETVDFIKMKDILSRRDISRTVMTEQLSEAFAIEKKKNKQVYEILKGFCGLAVSLNTLFSREEFDENHKKVQISFRDSKYDEKIEEYSSFLSEEEVELIGRIKQIHDLGVLSNVMKGSKYLSEARIREYDKHKQDLKILKRVIREYSPDKYDSMFRELEPGSYSAYVGSVNSGESHRRNVKNRQREDLYATIKKILKDIPDNEDKEYILKEMELEAFLPKQLTASNGIIPNQVHVIEMKKILSNAEGYLPFLKETDESNLTVSERILQLFEFQIPYYVGPLNLQHKNMPNGHAWAVRKKKGRVLPWNIEEMIDIKQSSEEFINRMVRHCTYLQGERAIPKDSLLYERYCVLNELNNLQIHGERISPQLKQNIFCTLFEKGKHVTQKQLFQYLVREGILEETELDAISGIDKDFKNALTSYGKFKAVFGESIKLDSTKKMVEDIIYWGTIYGDDKKFLREKIKDEYGECVTEEQLRRIAGFKFHDWGRLSRAFLQLSGCEKGIGEKLSLIDMMWENNYNLMELMSDKFTYQDSLAEMCENSEKVLKELEYEDLEDLYLSAPVKRMIWQTVLVLKDIEHVLGKEPSRVFVEMPREDGDKGVRKDSRKQKLKELYKGCKVDSKNWIEEIDGWSDQELRSKKLYLYYMQQGRCMYTGEIIDLHDLFNDNLYDIDHIYPRHFVKDDSLENNLVLVKKEVNAHKSDTYPIEKSIYDARHNWWRSLLTADMQSKFITREKYNRLVNRNEFTDEQLTGFISRQIVETRQGTKVVAHLFEEMFPTSEVVYVKAGNVSAFRQERKLLKSRVVNDFHHAQDAYLNIVVGNTYFVKFTRNPANFIREYRKNATANRYHINKMFDNTVERNGEIAWVAAEKNGASGTIQTVRAVMAKNSPLITRMNFESHGQIAEATLYGAKNANEESYIPLKESDERLQDVSKYGGFNSISITYFFLVEHTKGKKRIRTIEAMPLYLKDKIGTDKKALVKYCEEQLGLIEPDIRLEKIKIQSLIKRNGFYLYISGKSNKQLIVRNAVPLCLKQDWITYIKGVEKVNETGREYSLVTAEKNLELYEMLMDKHMTGVYAKRPNPVGEKLVHAKDRFEQLEVKEQCKVIMQILQLSQLSNMGADLSLIGEAKKTGICMISKNVSEADEFKLIHQSPSGLFETEIDLKTI

SEQ ID NO: 55 - protein, 1344 aa, synthetic construct
MEKFKKYNIGLDIGTTSVGWAVVETENQKILRKGNKPLWGIRLFEEASKAEVRRNFRSTRRRYDRRRQRISLLQEEFKEEINKVDSNFYIKLQESKYVTSDTINKTIKITDSEKEKLKKYLKECPTIYHLREKLINNTEKEDIRLVYLAIHHIIKYRGNFLYNSKGFNVNNLNIEEKIKEAFDTYFSYINTEYDDYSSLLDIKKLSEALMQQSKKDAREEIKNVLTESGLFENKIVNEFIKMMIGNKFKITTLLSLEDSDSKLEISFDGTEFEDKYDEIESITLDKIEILETMKELYDTLFLKKLFKGSNETNISSLMVKKYNEHKEDLKFLKKILKSDKELYKKFFKNDCIYEKYITNNITYDEFIKEVNNAISKVNIADYNLSNEYVTKIAPEIANGNFMPRITSTENGKYPYQLNKDELLKIIENQGKHYPFLLNKTNDGTYKIVKLLEFRIPYFVGPLVNSNQSKFAWMERNIQNVKITPYNFNEVINKELTAEKFIKRMISNCTYLLNEPALPNNSISYNNFKVLNELKQIRINGELLEINLQHKLIEELFKKTNGTITEKKFKDYLLSNNEFPMHGTELNITGYSADGAFANNMQSYIDFFGKNGIFEDTSYTINDAENIIEWITIFDDKDILEQKVRKKYPKLNDLKIKSLLSKKYKGWGSLSSKLLNTKYYIDKKSNTKKSILDLMYETKENFMQIINNDEYNFQQMINEINNTNNDIEKLSYKVVEPLATSPANKRGIYQALKIVDEIVNYIGYEPESIAIEMARNEDKKERKDNKKKYIENLYKKSKDEIENYNKLQKELNQEEINSQKMFLYFIQEGKCLYSGKSLNIEDLDSYEIDHIIPRTLIKDDSIDNKALVYRECNQNKAASYVLPQEYRNKYNQEWWKHLRKIGLMSAKKYHNLIRKEYSEEDIEGFINRQLVETRQITKHVANILNTYYKNTKVIYLKAKLSHDYRDRYDLFKFREINDYHHAHDAYLAAVLGEYKEKYLNKDVNFEVVKELNYMLINSGNKDKIKYGYVINSLNEQAHDLIFELSKKYMNEETGEVIFNAEEFNKRIEDTLYRNDILVSKKTEIRTGEFYNQTKNSKGNKGIELKKNMPTELYGSYTSLNPAYAIMVKYTKKNKTSQKLIGIPIYLTKQNDETIKDNYIRNILNISMNDSYEFTSKPIPFFSKLNWNGQICYLVGASDTVEVCNAQEFNYDKEFFKYHKLALQKLFNNKNLEINDNIYSNMLDEIIKYIVDKIEKQYKLFGNLIFELKIMINYDNIESNSVENKENIIKQLTKLLNCKSDNANFKFLNNKFSSAFGKKNGRTIEKFSLINLSVSEIYQKETIIGE

SEQ ID NO: 56 - protein, 1097 aa, synthetic construct
MRYVLGLDIGIASVGWAVIDLETLKVIDACSNIFAAAEAENNKDRRSFRQMRRIKRRMRTRISDFAKMWKLFGFNIPEEVNNMPLVLRVKGINEELSMDEIYAVLLNELKHRGISYLEDFGDNTNDGTNYSKSLEINQKELEDKYPCQIQYDRYCKYGNYRGQFVADINGEKVTLCNVFTSSSYRKEIIKILDTQKKYHSEINDEFIEKYMDIFNRKREYYVGPGNEYSRTDYGIYTTQLNEDGTYKTDENLFEKLIGKCSVYSEELRASAASYTAQEFNVLNDLNNLIINGRKLEEKEKYKIVDIIKSSDSINMRKIIKKAINEEIETLEGARIDNNEKEIFHCFETYNKMRKEFKKIDVDINSLSRDELDEIGKILTINTEKDMIVKKLSEMGLDDKVVDCLTNIRKNNGKLFTKWHSFSLKIMNELIPTMYEQPKEQMQLLTEMGVFKKQSDLFNNCKYIPSDIVKDDIYNPVVAKSVHIAIRIVNAVIKKYGMPEKVVVEMPRERNSEEQKKKIKDIQNKNKNELDGIIKKIDSEYDLKIGEKDFRNHKGLVLKLKLWNEQGGVCPYSGRKIDIFDLVKHPDMFEVDHIIPLSVSLVDSRINKTLVYKVENQEKGVNTPYMYLSKITRDWGWDEYKDFIHGLKIEKAKKVNYLFMEDITKYDVLKKFINRNLNDTRYASRIILNTLQDFFNANKTGTIVKVIRGNFTHQMRTNLNIEKDRDESFSHHAVDAMLLCYSQLGYDAFHKLQGSFIDFETGEILDKRMFNENMQEDVYKDYLYGNKWGQIKHHIEDAEKKVKYWHAVDTKCNRGLCNQTIYGTREIDGKTYKISRTADIRTKGGFDSLKKLLAEKNCKSKINNILMYRNDPKTFQFLMDIMEQYSDASNPFVKYEEETGDIIRKYSKKHNGPKITKISYIDNEVGSCIDISHKYGYEKASKKVILESLVPYRMDVYYNTKDERYYFVGLKQSDVKCKGNEKIINTENYKRSLVMEKMISDDQTIEDLEKQGFKFKLTFYKNDIIKYEKDGRIYVERFLSRNLSARNRIEIKPINAEKFDKPQVFVGLSNTKMVKKIRTDILGNYYECEQEKFSLICK

SEQ ID NO: 57 - protein, 1113 aa, synthetic construct
MGRLALGIDEGIASVGYGIIDLDMLEFVDYGVRLFEESTAADNKTRRTTRGHRRLISRKVTRLDDMKHLLKSLGILTDGFHPLNNVYEIRAKGLKEKLSNDELVAAILHITKRRGSTIETADDTEEASKETGELKEILQANSKKLAGGKFVCEVQLELLNNGKSIRGHHNNFKTKEYVKELKEILSHQELPEEACEAIIQIVSRRRAYYEGPGGRKSPTPYGRYIEANQGEPIDLIEKMRGKCSVYPDEPRAPKLAVTSDIFNLLNDLNNLAYDGDNKITMDQKDKVFKIVDKKAKVEPKDIAKIFGTDLSNISGFRIDKKKKPLKVEFKGYAKLKKCFKDCGETISLKDYEVLDQIIEILTNRKGIEERKQLLNLVKSSLITDKSVELLANLNGISAYHSVSLKAMRELNKEMYVTNYNQMQLLQQIHKGNDVSALKGKKYIEADDTAILSPVAKKSQRETFKVINELRREYGEFDAIVVEMAREKNSLDKKKRITERQKFFEEKNKEVDKLLKESGYDPGEINGKTKMKIRLYLEQDGKSAYTLEPLILKEVINDPDYTEIEHIIPISISLDDSYSNKVLATAAENKVKGNRTPIDAFNKHLFNDSDNGLHGDLQLYISSVKSSKKYSKKKKRYLLFKDDITKMDVIQKFIARNLVDTRYANRVVLNTLTDYFKANDIPTKVFTINGNITNIFRKKIHMTKDRDADFLHHAVDALIAASVKTLGLLNGYLGKYDLYDLYDEVTGEVKKVPDEDEFFDEKYITFISALKNIYDESSKFYRGIMTRNQLAYAPIKISHKVDTKPNRQVANDTIYSTRIVDGEEKRVMKFKDIYDPKFKELTEDVINNAAENKYIMAKKDPATFEEISRIILDHYETFKDDPKQYVMGKDGVKLKGENPLTAYKEEFGKIRKFSKKGNGSEVTSIKCYYETLGNCIDISKNYNLTQGKRVVLVQVSPYRTDFYRDVDEKIKIVTIRYKDVFFKENLGLYCIDHDWYKSEKSKKNISDKAVFLCSLHHNELIGIVKKEGQKYIYDDSRESDGNTKCHDGITPEILKFTATANDLENKIEVKPVYKKAKSRKSPSVGTFINVKKYATDVLGNLYEIKDNNLKLEFK

SEQ ID NO: 58 - protein, 1315 aa, synthetic construct
MEQKSLKKLIDYNIGLDIGTNSVGWAVTDFDNNILKHGNKNMWGARLFNEGDTAEQTRTFRGTRRRIERRKERINILQSLLLSDMEREYPNFFPMLRETSKVKEEKKEESLNGKKYNLFSELKFSDATYYAKYPTIYHLRRALIENNEKHDFRLVYLAIHHIIKYRGNFLYEGNLKNNSNEILESIDELLNFIQNTLEIVYVCRAEDIQNILQDKTKSKGEKKDDLMSCFDYEKDEKTIINGIICAILGYKFDVNKIFDTNIENSSITFSGEILNEEEIRDCLQDQAYVYDVLQKIYNWFILQDILQGNASISTAFVNKYEKYKKDLQNLKSLYREYLRNEYNDMFRKENNSSYASFDKNVSSCPIENLYKRIKLDLEKIPECKEKNDILKEIENQNFLIKINTTANAAIPYQLHYQELEKILTNQEPYYATIRENKQHILELMKFRIPYYVGPLAKEGESRFAWVVRNSKERILPWTFDDVVDVDATAEKFIKRMTNKCTYLINEDVIPKQSLLYSEFCVMNELANIRVNNHKLSSKEKKLIIDKLFKVKKTVKLKDLKQILLDYQVYSEITSITGLAEVDKFMSNMTSYIDMENILGEINETNIEMVENIIEWITIFEDKKILKRKIKKEYNLEDNIINQLSKKNYSGWSRLSKRLLVGLKSYDDGKNIMEKLQTSKDNFMQIINNDKYGFNKQIEELMQIQEKEMTYEAIEEIPTSPANKRAIWQSLKIVKEITKVMKKEPKNIYIEFAREEQEKKKRKDNRAKALLKIYDKYEEEIKMLRDYNPNVYKELKKKQNEKDFNERLYLYFTQNGKCMYSSRPLNIDTLYLYEVDHILPQSYIKDDSLSNKALVYKEENQRKSGNLLLDEKIINKQENWWKQLRKNGLIDEKKYFNLTRRKMFETDKEKIKFVSRQLIETRQSTKYITNLLVNQYPKTDVFAIRSNLTHNFRNFFEIYKNRNINDYHHAQDAYIISIIGNVIDKKMHYKDEYRYTEYVKNYIKKSEEELNKNKTWMIMGMVSNNIDKSKVKSTLCYKDCFITHKLEEQTGAFYNQTLYGPRDKKVTAVIPLKDNMSVEKYGGYSGENKAYFVIYSYTDKKNRQQTEMIGIPVKIAYDIKEKKITLSKYIKNKIKDGSNIKIIKSKILKYQEYLDENKEPMMLLSDSEIRINKQLVVTPEINRLIYVMNQEKTTEEDRKEIEEQIENIYDYLLDKLKKEYRFFNSIYTKLNAPETKELFEKLEYQDKVLTINGIIDLMHKGQGNLSKLKQGDRAGRKSGKTFKTETLNNMIFIDKSITGMYERRYKVNGMENSCSK

SEQ ID NO: 59 - protein, 1318 aa, synthetic construct
LKNEKNYNIGLDLGTGSVGWCVTDENNNIVKHNSKNMWGARIFEEAHTAETRRNFRSARRRADRRKERINILQSLLKDDMDKEYENFFPMLRNTYLVPEEKGFEYEMNGKKYNLFSDSNFTDENYYKKFPTIYHLRNYLIETTEKVDFRLVYLAIHHIIKYRGNFLHEGNFSNDNNSINESLIELTDFLKEKYDISLINDNEDIISILLNKNIKRQNKKQELLKMFDFDKEDKKVITNIVNAIFGYKFELSTIFDIEYDGKIDFSTEIVDEDSIIENLNEESSIYEILKSIYSWSVLQEILNYDDSANEENKKYISKAFMKKYDDYADDLKCLKEFYRKYLPKNKYKEMFKIYDEKKNNYVAYNGKNKGESNKKCDRYALYSYIKNDIDEVKESTDKKYIIDKMDNCDFLKKLNVTDNGAIPYQLHRDELLKIIENQEKYYKTLRENKTKIKDLLEFRIPYYVGPLEKNEENCNAWVVRKSNEKIRPWNFDDVVDKDATAEKFIRKMTNKCTYLINEDVMPKQSILYSKYCVLNELNNIKLDENRISKDTKKKVFCRLFLTKTKVSANDIKKLLAQDGLVIDNITGFTDESSKVFNSNMKSYIDLKSIIGDITENNFDDCEKMIYWVTIFEDKSILKKKLKENFEYLSDKQINKISKLNYSGWSRLSKKLINGLKSYDKNETIMEKLENTNMNFMQIINKDEFGFKKQIEDFMPKTEKGIHYKDIEDIPTSPANKRAIWQAMLVVKEIVKVMKCEPQNIFIEFARNEGKKGEITKKRFNKIIDIYNEIDNIKKNDELYKELKSHQSDKEFSERLYLYFIQLGKSLYSGKPIEINRLSEYQVDHIIPRSYTKDDSIDNKALVYADENQRKADDLLLDSSIQRNMRSWWEYLLKNGLISPAKFNRLTKTKMFTNDDDRSKFVKRQLVETRQITKYVTNILNNNYKNTNVFSLRSNITHNFRNQYKIYKNRNVNDCHHAQDAYIMALTGNILNKEWKNINEFKYGKYVKDYMNDKNFSKNKNGIIVGFISKYVDAGKLRKVMEYKDFYISTMLMEQTGAFYKQTICSPKKHPTISLKNGLDTSKYGGYTNEFKAYCAIYKYLDKKKIERYRMIGIPVKIASDIKNNKITLQQYLEEYSKEKNEVFVNIFKSKIMKYQEYLDENGNKMILKSDSEIKSNKQLILSLDMNELIYAINNDQADKEEYLNKYGNVFEFVYDKLAEKMHIEYPIFETNYKKIIESKNTFNELDLKDKKDTINGIINLMSGEEGNLTKIGLGDRTGRMSKQKFEDKRVRNIIFIEKSVTGMYERRYKIDGMENSCSN

SEQ ID NO: 60 - protein, 1100 aa, synthetic construct
MGVRFGLDIGIASVGWAVVSDEYQILESGANLFEAAEASKNADRRTYRQTKRLLRRRRTRVKDFEKLWIKYGLPVPEHMCNNQLDMRIKGQHEELSKEEIYFVLKYMLLHRGISYLDDVVDDTNVTGEYKKGLLYNQKELNENKLPCEIQYERLCKYGKYRGEITTTNNEEKITFSNVFTKSAYKTEITNFLDMQKKFHPFISAGFISEYMVIFERKREYYEGPGNEKSRTDYGKYTTLINPETGQYITEENIFEKLIGKCSIYTEERRISAASYTAQEFNLLNDLNNINVNNRKLSKEEKCAIICKVKTEKVVNMKRIISDVMGEKIEYLTGCRIDKNGKEIFHKMEQYNKIRREFEKVGLDVDYFSRDELDQIGDILTLNTEKEAILCAFKRNGVVMSTEMQETLMEIRRKNASMFSKWHKFSKKLMLELIPELYEQPKNQMQLLSEMGVFKLDTEKYKQYSKLPKEEILDDIYNPVVRRSVSVTIDIINALIKKYGWPKEIVIEMPRDRNTEEQKQRIRDFQKNNEKELKAIIKKVKDEYGYNITEDHFYKHKKLAMKLKLWNEQDGLCLYSGKTIDIYDLVNNRNAFEIDHIIPRSISFDDSRTNKVLVYATENQLKGNQTPYMYLRETKKMWGLDDFVDYCCTLKDKKKISKTKLDKLLFREDITKIDVLKRFISRNINDTRYASRVVLNSIQSFIKAKESETKVKVVRGSFTSQMRMAMQLPKDREKDYSHHAVDAMLICFSQMGYEAYHLIQKEFIDFENEEITNYSLWSKNMDDATYQKVMFSQRWKLIRDNILNAEKKVKYWHRVDKKANRQLCRQTIRGTRRIDDKNMIVYKLNIYDKTGYDTLKKKLSNGKESDFLMFHNDPKTWEQLKMVLNEYSDAKNAFVQYELETGDCFRKYSKKHNGPKVTNLKYISGEVGSCIDISHKYNLSPNCQKVVLENINPYRTDVYYSQKEKRYFLVGLKYSDFKYTSEGYRIDEKAYCNALKNEEMLQSGMSLKDLSRQGFEYLFSLYKNDYIEYELKGKVYVERFLSRTMPQKANYIETKPIECANYEKRHPVGLKKAISIKKIRTDILGNRYYAQKEKFTLDVDN

SEQ ID NO: 61 - protein, 1358 aa, synthetic construct
MEKEYYLGLDMGTNSVGWAVTDDAYNLIRFKGKDMWGIREFDEANPAVERRTHRTSRRRKQRNAVRMGLIRSYFADEIQKVDSNFYQRLDNSFFHVEDKQDNVKDKNGIFNDKNYKDADYYKQYPTIFHLRKELIEDASQKDVRLVYLAVANMFKHRGHFLLSGIGSESSKQTITDLYNQVSGLITEEYGISLKTDIAGTDFEQVLCSREYSRTRKSEKVCEILEIKNSDKLKKAFVKSMCGLSVKITDIIDDTFTEISDEDKKITFSFVDSDYDEKAGVLEEKLGTDVYDIISKLKEIHDKAQVAEIMKNYQYLSFARVADYEKHKKDLKILKRVVKKYFEPTVYDKLFRSEENGTYSAYVNSFNSEEKSRRNYKERKQEDLYNSIKKLLKSVDNVDDTDIEYIKNEMDKENFLPKQLTFKNGVIPNQIHASELKAILKNAEKYLPFLNEKDESGYTVSERILMLFSFQIPYYVGPVSSNSNGWVKRLEPGQVLPWNIDKKIDLKETSEQFIQRMVRNCTYIAGEKVLPKASLLYENFKVLNEINNLSINGERITVNLKQDLYNTLFLKGKRVTRKQIFNYFHSRGLVEEDSQISGIDIAVNNSLSTYGKFLPLFGEHLKEDKYKKMVDDIVFWCTVYGDSRKFLREQLEEKYSDVLDENQIKRITGMKFKDWGNLSKEFLELSGCCKSTGEVMPLIRAMWETNYNLMELLHSEEFTYSEELEEKQGKIEKVLSEYTIDDLNEEYFSAPVKRMVWQTLLIIKEIEKIMKCPPTRIFVEMTRKDDEKGDNGRKSSRKDQLLALYKKIQSEDKEWKKEMSSRIEDYDKNGALRSKKLYLYFIQKGLDMYTGKPIDLDNLMSANSNYDIDHIYPRHFVKDDNINNNLVLASKPANAEKSDIYPIKDSIYQKMKYTWKVLRDEGLITEEKYKRLTGRNPFTDEQKAGFIARQLVETGQGTKGVADLLTALLPDTKLVYSKAGNVSDFRKRYDIPKSRLINDFHHAHDAYLNIVVGNIYFTKFTSNPLNYIRKECSSKDNKYQYNLGNMYKWDVKRDGKTAWVAPEEGETGTLATVKKMLSRNTPILTRLTIEGHGGIADATLYSHKKAKQDTYIPLKSSDARMADVTKYGGYSSVTTAYLFLVEHEIKGKKIRTLETVPIYLKDRLENSKEKLEEYCRETLGYTNPSVRMKKIKLQSLIKLNGYYMQISGRTGNRFTLRNYVQLCLNGKWVDYIHKLEKYQDKGVIDDLITDERNVELYNVLCEKHSVGIFTLRPNLVGDKIKNGYDKFVNLTTEEQIKLLIEIIKLTCVGIVSANLTLIGGAAISGKMLMSKNITYANECILINQSVTGLYENRIDLLRV

SEQ ID NO: 62 - protein, 1360 aa, synthetic construct
MKYYIGLDMGTSSLGWAVTDDKYRLLRKKGKDMWGVRLFDEADTAAARRTNRVAKRRRNREKARIGYLKELFATEIEKIDSGFYQRLDDSKFWEEDKTVQQKFALFADTGYTDVDYYKEYPTIFHLRKKLLESTEPQDVRLVYLAILNIYKHRGHFLNANLDENGNDDFADIYANFVKMAKDVVDIDFSQDISDNWLRETLSSSELSKTGKHSAIIEHYGIKKSAEKKKVEMLKLICGLKGVLSKIFEDTEWGDEDKKFSISYRDSNYDETSQKAEELLSESAYELFCTMKQMHDWSLLANIMKGEDGEYKYLSLARVAAYEKHQKDLKILKKLYKDNDLKKYNEMFRIMNKNNYSGYVGSVNSDKDKTRRGAQCSRDDLFKKIKEDIKDFAETAEKNYVLEEIGKENFLPKQLTASNGVIPNQIHKKELKKILENAENYLPFLREKDESNLTVSERIVKMFEFQIPYYIGPISPIVNGDMKYSKNVWSVRKEAGKVYPWNFEQKIDTEESAQRFISRMVKHCTYIGGEEVLPKNSLLYEKFMVLNELNNLRINGERISVKLKQDIYEYLLKKEKKVTKSKLIEFLRIHGEVEPQATPEISGIDDGFTNKRANYCKFCRLFGVDTLTYEQEKIAEQIISWSTVYGDTKKFVKKKIQDNYGDVLSDKQIAQAAGYKFKDWGRLSEELLKVEGIDKSTGEESTVIQKMWSDNYNLMELIESDNFTYKQNIEERAKVIDKTLSEIVYEDLDELYISAPVRRMTWQTILVLKEITEVMGEAPARVFVEMARDPNAEKKRTESRKNKFLALYKNCKKEERELYNQINKRDEADFRSKKLYLYYTQKGRCMYSGERIDLDDLFNDNLYDIDHIYPRHFVKDDSIDNNLVLVKKEINAHKSDGYPLADGIQKKCRVFWNELKSGGFITAEKYERLTRTFEFTEDERVSFINRQLVETRQGTKAITDLFKKTFPQADIVYVKAPNVSAFRQKYKLLKCRNVNDFHHAQDAYLNIAVGNVYHVKFTKNPYNFIREYKKAPEKNPYHMDKIFNYPVVRNDEVAWQTKNDESITMVKSMMKKNTPLVTRRNFEVHGALENKLGIKSAQLAKKANGVGYIGVKTSDARLQNVCRYGGVTDIKGAYFCLVEHTQKKKRIRTIEVVPLYLKHQLDTKEKLEQYFAESYGYENPNVRIEKIKMYSLIKVNGFYMYLTGRSKKRLLVCNAVQMVLTQEWMMYIKEITKAYNAQYSDEYMQKQKLISKLKNEKLYDVLCEKHLHSIYRLRPNGVGQKLLEGRQKFEEISLLEQIYVILQILQLSQLINNGANLDIIEKGAHAGVATLNKMISECNEFKLINQSITGLYENEVDLLTI

SEQ ID NO: 63 - protein, 1339 aa, synthetic construct
MEKIKKYNIGLDIGTNSVGWAVVESGQQKIIKKGGKALWGVRLFDAASTAKDRRLKRSIRRRYDRRRQRIKLLQDEFANEINKVDSNFYKKLQDSFISPNDKNNSKTELTDFDKFNIFSNNIRNKINNISEEKKYPTIYHLRNDLVNSKDKKDIRLVYLAIHHIIKYRGNFLYENSDFNVNNINLKEKIEETFNHYIEECMPGEFNIPINYEKIANILLKDSKNEIKDLLKDELLVIDTYNKGFSSEFAKMIVGNKFKIERLIPVSNEEKSMSISFNGNDFDEEYEKLEELLGQNIEILDELKELYDNLFLKRLFKGSESNNISSLMISIYDEHKKDLKMLKKIFKNNKKIYNELFKGENCKYEEYIHNKITQEDFCKIILNSYEKLGMSEETIEFYNKNLKSKIENNSLLPRISEPSNGKYPFQLNRNELIKIIDNQKEYYKFLGEKINNKYKLVKLLEFKIPYYVGPLVSSDKSCFAWMERNNNEKITPYNFDEVVNKELTAEKFIKRMISHCTYLINEPALPSNSLLYNKFKVLNELKQIKVNDRKMTLEQQQNAIKDLFEKTSGSITEKKFINYLKQTKEWDMYSEINVKGYSADKKFANNMQSYVDFFGKNGIFDNTNYNEENAEEIIEWITIFNDKEILESKVRDTYPDLTDDQVTKILRKKYSGWGELSKKLLTLPYYKDKETEVYKSIIDLMFETKENFMQILNNDEYKFQNMIKENNKITNTKELNYKDTVSELATSPAVKRGIFQSLKIIEEIVDYMGYEPDSVSIEMSRGDDKKERKPDRKKYLTELYKKSKDSIENYNTLNKQLVEKEIDSQKLFLYFIQEGKCMYCGKSLNVEDMISYEIDHIIPRTLIKDDSIDNKVLVHKECNQNKAASFVLPPYYRSDYNKKWWTRLKNIGLVSNKKYNNLVREKYSEDDINGFINRQLVETRQIIKHVANIIENYYDTKVIYLKTNLSHNYRERYELFKFRELNDYHHAHDAYLAAVLGEYKEKYMKYNITYDLVRTLNNKIKEKGQTEKLKYGFIINSLDSDVSSVIDGLNKNKINKETGEMIFSPDAFNKTIENNLYRNDILISYKTEYNTGEFYNQTIYGKGKGNIRLKDNLPTEIYGGYSGVNVSYFMLVEYKGKRKIIGIPILIAKNNNEKDLNDYIINQINFSEYRVLKNKIPYNTLFEYKGQQVYIKGYSSKNKNGEISNAIQLKISKELYLKWKYALNRILNNKENNLNESEYKENLIEILNYLLQLNDYYPLFDNEIIQIKKYVEKNYQNLGIKDFEKIIKQLLILYKCSSENANLKEFGLKDRIGRLTGKVISEGTIIYKSITGIREKKYEF

SEQ ID NO: 64 - protein, 1119 aa, synthetic construct
MNNNYVLGLDIGITSVGYGVIDIENGEFVDYGVRLFEEGTPAENKKRRDSRSRRRLTARRHNRIHDMQKLLKENGIMLDSYHPFQNIYELRCKGLTKELTKDELTAVILNITKHRGSVIETVEEDASKSEDELSLKATLQQNEQLLKNGKFVCQIQLDRLNNKNSVRGHENNFSTKDYLRELEVILQHQNLNDDLINKILDIVGRRRAYYEGPGSEKSPTPYGRFVEVDGQIQYFDMIEKMRGKCSVFPEELRAPKMSIRADLFNILNDFNNLSVNGNKLTVEEKERLLKIISKKGKISLGELAKELDVDKNSIKGFRVDKKDKPIFTEFKGYKKIKSILEKEKPISLEDCEMIDSVIEILTNKKGIQERKEALQHLEYHFSNAVVETLANTTGITGYHSLSFKALRLLNKELFESEMNQMQLLHELKLFDKNRVSYKGKKRIEANSDAILSPVAKRAQNETFKVVNALRKKYGEFDSIVVEMTRDKNSKEKQKRIKAFQSMREEGSQNMNKMLEEWGYDPSEVNGKTRMKIRLYEQQNGKSAYTLQPLDLNRVIKDPSYTEIDHIIPISISLDDSQNNKVLALRSENQMKGNLTPFLAYSSNKFQGMGCSYSEYKTNVLSNKNIPYRKRLNLLNEEDIIKEDIAKKFINRNLVDTSYACRVVLNTLSNYFKDNEIDTKVHTINGRVTDIFRKKIHLEKQRDEDYLHHAIDALIVASIKKMNLLNGYLAKQDHDFNDLYNEETGEIRTIPGDKVFFDAKYIQYIINLKTIYQESSQYYNHVIDKEAMNFNPIKISYKIDTKPNRQLFDEQIKGVRTFDNKKYVIEKISDIYNPKENTKLIEDILNNNCEKYFINKNDPRTFEVIKNIVKEDYEIYQSNKEIYKVQNGKIKYVNKYEKDVDTKKVKIENPLKKFYEENGAITKFSNKKMGPPVKSLKYKGKLLKEHAEAIDHTQNRTIGVKLKLKPYRTDFYKCKDGKVRFVTIRYMDVEYKKNDNMYHINQETYEELLTRKKIDSHAIFLFSLHTNELLMIERDEGTNFYFNPNKVSSLKYDGKNFELFMFLGTNNDSMNRIEVKPRYKFTKDRINPITTTFLQCKKMATDVLGNLYEMKDNKLKLDFK

SEQ ID NO: 65 - protein, 1113 aa, synthetic construct
MGRLVLGLDIGITSVGYGVIDIDKYEFVDYGVRLFKEGTAADNEIRRNTRGHRRLISRKATRINDMKHLLKSLGIINEDFHSLDNVYEIRVKGLKEKLSNDELATAILHITKRRGSTIETVDDTEEASKETGELKEILQANSKKLADGKFICEVQLELLNNGESIRGHHNNFKTKDYIKELNEILSHQELPEEACEAIVQIVSRKRAYYEGPGSQKSPTPYGRYIEANQKEPIDLIEKMRGKCSVYPDEPRAPKLAVSSDIFNLLNDLNNLTYDGNRKITMDQKDEVFKIVDKKAKVEPKDIAKILGTDLSNISGFRIDKKKNPLKSEFKGYAKLKKCFKECGETISLKDYEALDQIIEILTNRKGIEERKQLLNLVNSSLITDETVNKLASLDGIANYHSLSLKAMRELNEEMYVSNYNQMQLIQQIHKGKDVSALKGKKSIEADDTAILSPVTKRAQRETFKVINALRRKYGEFDTIVVEMTREKNSADQKKSINNRQKFYEEKNKEVDTLLKENGYNPDKINGKTKMKVRLYLEQDGKSAYTREPLSLGEVITNPRYTEIDHIIPISISLDDSYNNKILATANENKAKGNRTPIDAYNKHLFDNPTLGLSGDLQTYISVVQSSKRFSRRKKNYLLYKDDITKMDVVQKFIARNLIDTSYANRVVLNTLTDYFKANEIPTKVFTLNGNITHKFRTQINLPKDREEDYLHHAVDGLIIASVKTMGLLNGCLAKYELKDLYDKDTGEVKKVPGKEEFFDPKYIEFISTLKNIYDESNKYYRGIMTRDRLAYAPIKISHKVDTKPNRQVADETIYSTRVVDGQEILVQKYKDIYDPKFKNLVEDIVNGKAEDKYIMAKKDPATFEEIVRIVLNHYETFKDDPKQYVKDKKGVKLKGENPLTAYKEEFGKIRKFSKKGNGPEITCIKYYAEKLGNHIDISKNYDTKHGKKVILKQISPYRTDFYRDTDGKVKMITIRYKDVSFKESLGLYCIDRDWYQTEKTKKKISDEAVFLCSLHHDELVGIVKKEGQKYIYDDSTESDGNTKYHDGVTPEVVKFTATNNDIKNIVEFKPRYTKSKKQLMVAIGTFVDVKKYATDVLGNLYEVKDNNLKLEFK

SEQ ID NO: 66 - protein, 1315 aa, synthetic construct
MENKKNKKIKEYNIGLDIGTNSVGWAVTDLDNNILKHNSKNMWGARLFEEGKTAAETRSFRGARRRIERRRERINILQSLMLDDVEKEYPNFFPMLRETSKIKEEKNAEAINGKKYNLFSELNFSDATYYKEYPTIYHLREDLIKTQKKLDIRLVYLAIHHIIKYRGNFLYEGDLKSSNDKILDSVNILVSFIQENLDINFNSKAENICRILMNKEKTKVEKKDELLKLFDYDKSEKSIISNIISAILGYKFDVNKIFESNLDNSSISFSNEIANEEEIKEAIKEQCEIYEILQRIYSWFVLQDILQGNTNISTAFINKYNKYKKDLKLLKKVYKDNLKKEYNKMFRIEDNNSYAVYDKNISKCSRENLYKRIKQDLDGIDGCKEKEEILKYIENDEFLVKINTTENAAIPYQLHYQELEKILENQSRYYLTLKENKQHILELMKFRIPYYIGPLAKSGDGKFSWIVRNSDEKILPWTFEKIVDTDKTAEKFIKRMTNKCTYLLNEDVIPKQSILYSEFCVLNELANIKVNNHKLAPKEKEMIIEKLFKYRKTVKLKEFKQFLIDYQIYKEVDSIIGLAEEDRFISNMSSYIEMKEILGNIDEKNIEKVENIIEWITIFEDKKILKRKIKKEYHLDDNTIEKLAKKNYNGWSRLSKKLLTEMKSYDDNKSVMEKLKSTRENFMQIINNTKYGFDKQIEEKMREKQKDNITYEEVNEIPTSPANKKAIWQCLKIVKEITKIMKREPKNIYIEFAREEQEKKKRNDTRAKQLLKIYEKYEEQIQILRDYNPKIYKELKNKQNDKDFNERLYLYFTQNGRCMYSSKPLNIDTLHLYEVDHILPQSYVKDDSLSNKVLVYKKENQRKSDSLLLNEEIINRQQTWWKQLRKSGLIDDKKYNNLIRRKMFETNYDKVTFISRQLVETRQSTKYIANLLLAQYKNTDVFAIRSQLTHRFREYFEIYKNRNINDYHHAQDAYIISVIGNVIDTKFYFRNEYKYAEYVKSYIKKEEEEKDKKNKPWIIMKMISNNINHDEVKRTLNYKDCYVTRKLEEQTGAFYNQTLYSPKDKNALIPLKENMQAEKYGGYSNEYKAYFVIYSYIDNKGKKQVEMIGVPVKIAYDIKNKRITLVDYITTIVKNISEIKIIKGKILKYQEYINKENEPMVLLSDSEVRTNKQLIVVPMINRLIYIMNKEKITEEEKEEIQKEMQNIYDYLLEKLKNEYKVYGSIYQKLNDKKTRKVYENLTYEDKKIIINGLIDLMHKGQGNLKKLNQGDRAGRKTGKSFKTKELSEMTFVDKSITGMYERRYRVNGMEDHCSN

SEQ ID NO: 67 - protein, 1338 aa, synthetic construct
MKKEYNIGLDIGTTSVGWAVVEVDNQKVMKKGKKALWGARLFEEADTAADRRTSRSTRRRYDRRRERIKLLQEEFKDEINKVDQEFFTKLQNSKYSKKDKNNKRLRYSEEEINGAAAYYKVYPTIYHLRKALMEEQKQFDIRLVYLAIHHIIKYRGNFLYEGKNFSVKDISLEDQLRKLIELLIKELPELELTEDDAMDIDFKEFAEAFFKETRNDVKVTLKSILKSIDSTFASEFSKLMVGNKANIPKMLKQDSSEKIEVTFLKSDYENKYDKCLTALGEKIEIYDLIKKIYDSVTLKKILGKEDNPSISNKMIKMYDKFSKDFRWLKETLRKDREVYNAFFKSSKGEKSPYEKYMSSDIDNNELKNSIDKSISALIEHNNISQENLDKYKNEILPQLENNTFLCKTNSTDNGIYPYQLNENELIQIIENQGKYYPFLLEKYNEDTYRIVRLLEFKIPYYIGPLVTKEKSKNAWLIKTTKTGKITPFNFDTMIDKEKTAIEFIKRMSSHCSYLLDEYAMPTNSILYSEFKVRNELKQIKINEKSIDLNLQQKIFEEFFKTTTGSITERKFINYIKTTGEFSMYNDDIKITGFSSTNSFANNMQTYIDFFGTNGIFENTKYNIADAEQIIEWITIFEDKGILEKTVKDNYPLLSNEQIQLILNKKYKGWGNLSKKLLTTKYYKDKQTEVYKSIMDLLKETKENFMQIINNKEYKFQKMIEEYNNNSNATLSKLEIVNKLATSPANKKGIYQALKVVDEIVGYLGYEPKNIMIEMSREEGKKVRTVDRKSKLQNLYNECKNQIVDYKRLRHELDSRNITTEKLYLYFIQEGKCLYSGKPLEIEKIEDVSMYEVDHIIPRSLVKDDSIDNKALVIRKYNQEKKDNLVLPKAYRNLAQIQWWQRLKNNGLMSAKKFHNLTRDEYSDEDIDGFINRQLVETRQIIKHVANILGNEYRKTKILYPKASLSHNYRERYELYKFRNINDYHHAHDAYLAAVLGEYSDKYMKKSFDFSQIKEINKKIIELDEKEKIKYGYVINSLDERVSNIVINSSKNMTDEKTGEVLFDAKKFNETVAKNLYRNDILISRKTEIRSGQFYKKTIYKRCVGTIPIKKDMPVDIYGGYSNNETKYLKLVKYGNGKKKLIGIPMNLAALNDTKIIDNYIKYQLKLKNSDTYEVLKNKIPFETEIIYNNQNVYIKGYSVMGRTCEVSNAHELKIKKDLLYNWRYALNYFINGKEKDVEEAKKHSIEIYNFLVESIDYPLFNNELKKVRENIKFDTLEIEKQKKVILELFKLFKCSSTNANLKEFGLSDRIGRLSGINIKDGYIITKSTTGLKEFKYKI

SEQ ID NO: 68 - protein, 1123 aa, synthetic construct
MEGITMDNKYVLGLDIGISSCGYGIIDLETGKFVDYGVRLFKEGTAEDNGKRRDARSRRRLTSRRHTRIMDMQKLLKENGIMSNDYHPLQNVYELRCKGLSEKLTNDELTAAILHITKHRGSVIETVEEDAKKSEDELSLKATLQHNEQLIKQGKYICQIQLDNLNNRNHIRGHENNFSTKDYVNELNEILHHQELDEQFINKIIDIVARRRAYYEGPGSEKSPTPYGRFIEVDGQIKEIDLIEKMRGKCSVFPDELRAPKMAVTADLFNFLNDMNNLTVDGNKLCPEEKKGILTIVSQKGNITLRQLAKELNVDEVDIKGYRIDKNQKAIFTEFKGYKKIKSILEKEGYSISLNDYEMLDRIIEILTNKKGIQERKDYLYHLEYRLSDSVVDTLANTTGITGYHSLSFKALKLLNKELFESEMNQMQLLHELKLFDKNRVSHKGKKNIESDPEAILSPVAKRAQNETFKVVNALRKKYGEFDSIVVEMTRDKNSDEKKKRISNYQKIRESGSKEMDKMLIEKGYDPTKINGKTRMKIRLYEQQGGKSAYTFEPLDLNRVIKDSKYTEIDHIIPISISLDDSQNNKVLALHSENQVKGNLTPFMAYAANKFNGMGCSYSEFKKNVLSNDNIPYKKKLNLLNEENITKEEVAKKFINRNLVDTSYACRVVLNTLSDYFKDNEINTKVHTINGRVTDLFRKKIHLEKDRDEDYLHHAVDALIVASVKKMNLLNGYLAKQEHDFNDLYNEETGEIITIPDDKVFYDERYIQYIVNLKTLYQESNQYYNRMIDKEDMHFNPIKISHKIDTKPNRQLFNEQIKGFRKIEGKNYVIEKISDIYNSKENIKLIEDILNGNCEKYFIAKNDPRTFDIIKNIVLADFEIYKDNKDIYKIDKNKIKYVNAYKADKETKEIRIKNPLTKFFEENGPVTKFSPKRKGPPIKSMKYKGKILNEHVEAVDRTQNKLLGARLTLKPYRTDFYRCEDGKIRFVTVRHVDVEYKKSDGMYHIDNGLYNELLNKKKIGSQAEFLFSLHTDELLYIKKEIGESFCFSPKEKFVYKDSDYELFTFLGTNNDVRNVIEVKPRYMFTKTRINPYTTTFVECKKYATDVLGNLYEVKDNKLKLEFK

SEQ ID NO: 69 - protein, 1219 aa, synthetic construct
MKYNYILGLDVGISSVGWGLLALDENNNPYKIIDVGSRIFPPGEVEKTGDSRAKERREKRGARRVTRRREFRIDRVRNLLFEEGYLKGNITKSKVSEKNEELTIIYNDMINSYYKNKNTNPYKLKVEALDRKLTNDELCIILVHYAKKRGYKSNREESADKENGKVLSAIKENEEIIKTKHYRSISEMYIKDEKFKDKIKNSAGNYKISVTNEMYLDEINKVLDKQITFGLITNEFKQKYLEIYNSRRHYSEGPGNYYTYDESGKRITNRSKYGGNLIEKMIGKCAFDKKPRAPKYAPSSELFVAISKLVNLRYKQENEQYTKLSSEEIKQIIAEAKQKNKITYKTLIKILNKEKIKIKDLNLSKKEYAKVIEELKNQLKISKDEKLDIYSLEDSKREIYEKIYNDKLYSKALIELKGYHTLRKVITDCYDKSIWTEVSNNIEFLDELALYCTNYKLNDEILKSIKESQIIEEKFEDYNFVEKLPNFKDHLMLSTEIIRKIIPLMEEGNTYDKAMAKIGYDHSNLYDKIEKKDLLVPVCVDRNITNQRVIRSLTQTRKVINAIIKKYGTPKIVNVETARELAKSRRERNEISKNQEQKQEENEQDKNHLVELGFFSDINKISSNDLLKYRLWKEQKEFCGYSMEKIKIEDLFRNNIVQIDHILPYSRTYNDNYLNKTLVYTKYNQDKGNKTPYEWFGKTSKWEEYESYINNLSISQKKKDNYLLKKLDFETEREMREQNLNDTKYISKELATLLKSHLNVEEVNTYPGAITSKLRARWGFNRLTHSYISKTYIMPENMKKDINKDRDNHLHHAMDALVIASITKSLQQKITLYEKFSRYIDGKLKEKISDITINDEAITYDNETGEVISTNYKEYLKEQLSKDNIFYNKHNIGKLKFPLPYENFVQEAEARVYEQDINTLRYELQNLRTYTKKELKNVHILTPSLAKTKATGAMHKETYYGIYEEKDKDETIRTWKTLRTPIEKVKRKDLENIQGKDGGSKDIYNALVEWFGKDETGEKALKRHEGKYPINPNDKEQKEIKKVKIYTEYKNTGHMIKDANVEKGGIYRIEIYKSKDEKDNKLYFAAYDMYEIKTIERIKKSTKENKPVPALRVKVDYGRDKNTMIIPYEKLTTDYKLYKVLNKSDLIKVILKNGKESMGYVVGFTSGMLEIKSIIGDGYDLISEGNIFTQFRTQYTITVSTIKEIEKLSISILGEISGL

SEQ ID NO: 70 - protein, 1205 aa, synthetic construct
MKKEKYVLGLDIGISSVGWAAVRLDENDNPIRIIDANSKIFSPGEVPKTGASKNLIRRTKRDTGRVIRRRSFRIDRVRHLLNLNGYLGDDIIEGLVSDVNDKLTLIYSNMLNKYYKNKNTNPYELKVAALDRKLTPDELSIILVHYAKHRGYRSNREDNTDDKETGKVKKSITDNEELMQNKNYRIISEMFLKDEKFKDRIRNSTDDYKMCVSREMYQKEIEIVLDSQIKYGLITEEFKNDYIKIWSSQRHYAKGPGKPSKYGGDLIVRMTGKCKFDNNLRAPKYAPSSEIFVALTNLVNLRYHKFGNINYIGLTKEEIEKVIALAKSKDNVTYASLFKELGYTNVTIKGLSLSKKEYNKALTKFKSKYKIDEKSTIDFNTLSGEKKIEFNELKTKEVLSRNFISLKAYSDMRKAFSTKLNTEEWYKISDNYELLDTIATILTNYKLNEDVEKAIKENNIDERYKDIIKSLPNYKEHMMLSLDLIRKLNKIMITGKTYDTAMEELGYNHSLVNENSVKEDLLIPINQDNELTNQRVIRSLSQARSIINSVIKKYGMPERINIETARELAKSMEERREIENKQKENHENNEKTREEIFNLGIFKSKDDIKSFDLLKYKLWKEQLEHCTYSGEPITIEDLFDNNIVQVDHILPYSRTFDDTYFNKTLVLSKYNQDKKNNTPYEWFGKDKEKWNNFVGYISSLNIPTKKKDNYLLTNLTPEMEAEYRNQNLNDTKYIARYLVSYIKANLNVKKVGCPSGSITGKLRNYWQLNGLTHSLESETYYVKNRDENTKKNRENHLHHAMDAILIAITNEKIIKRVSDYERYKRYLDNKPISYIINYAKSMDDSFEINNFTDEETGELYENSLKKYIADLRTNEFFQTNKKNNTIKTYLPTPYADFREEAIVRVYEQNKEILRNKLRAFHYNEEDLENAVPLIPKFAKNKVGGPLHGETYYGLKTYKDGNETKYYKTSRISLNSDKFTKDKLDNIIDKDSGSRIVYDTISAWLENDNGKEAFAKHNGYPINPTTNAPIKKIKIKSDYDGKGHIIGNKIVEKEDIYQIDIYKKKDEDRLYFVAYDAMELYQIKERKDKNGNIIKPLYNIPVDLWWGQGKNHKKMKYLDLQENYEFYVSLQKNEFIEIILNNGNKGYAYVKGFSSGQFEVGSALGDSLDIIGEKNLFQGARERYQLTISTIKSIKKVKLNNLGKIE

SEQ ID NO: 71 - protein, 1105 aa, synthetic construct
MKVHFGLDIGIASVGWAVVNENYEILEAASNIFSSADASKNVERRNFRQSKRLLRRRRTRILDFNKLWCANGMTIPETRCNNQLELRVLGLKEELSQEEIYYVLSNMLTHRGISYLEDAIDDSNAGKSDYEKGLIYNQKELDTKYPCEVQFERLQKYGKYRGHIVVNDNNGEKITLSNIFTISAYEREIVDFFNEQRCYHNFLTDDFEEEYLDIFRRKREYYEGPGNEKSRTDYGKYTTKINPENGEYLTEDNIFEKLIGKCSVYPQFRRAAAASYTAQEFNVLNDLNNLIINGRKLKQEEKEEIIRELLSAPARIDMRKIIKKVIGEDIESFSGARIDKDEKEIFHTFEVYRKMKKAFMDKDLSIDRFEMNQLDLIGEILTINTDKKSIVEAFKREQEKIQATDEEIDIFVEFRKKNNQLFSKWHAFSIQIMDELIPEMYNQPKNQMELLTEMGVFRNKGKAFAEMTSLTPNVVTEEIYNPVVKRSVRVAVDIINALVKKYGYPEEIVIEMPRDNNSEEEQKRIKDTQKKNEKELADIISKVKKEYGRDITGADFREHKKLALKLKLWNEQQERCLYSGKVISINDLLDNPNLFEIDHIIPRSISFDNSRSNKVLVYATENQKKGNFTPYMYLSAVNREWNYEKYKAYVLSLKASKSIGSGKINNLLYTEDITKVQVLKGFISRNINDTRYASRTILNVLQAYFKEKNPDTKIKVVRGGFTHQMRENLKLDKDREKSYAHHAVDAMLICYSQMGYEAYHKLQSQFIDFEEERIIDKNAWQQQMNDRVYEDTLYQNKWQQIKSNIVEAEKHVKYWHKVDKKANRGLCNQTIRGTRVSEGEVYKINKINIYTKDGIKTFRNMIEGGKEKSFLMYENDPKTFENLMIIYNQYKDTDNPFAAYEKETGDFVRKYSKNHNGPRIECIKYRDGIVNSCIDISHKYGYEKGSKKVILESLKPYRMDVYYNKEKQQYYFIGIKYSDCKFEKGGYTISEEAYTKALISEKIIKEGQSRKDLDSLGWEYQFSFYENDIIEYEKDGIIYTERFLSRTMPQARNYIETKPIDAPKFEKQNLVGLSKTTSVRKLYMDILGNWHYCRGEKFKLHIDNV

SEQ ID NO: 72 - protein, 1104 aa, synthetic construct
MMEFKVALDIGVASVGWAIVQNDLKVVDSGARLFDSADPSKNVERRDNRGTRRLLRRKAHRLTVFDRFWVGKGLDSMDEITENSLILRNKAIIERIDIKEVYCVLRNMLKHRGISYLDDAIDDNVKGAYKEALNNNALELKVKFPCEIQLERFNEFGKFRGDVIIELDKKEQRALSNVFTKSGYRREIEQFFEVQGAYYPFMDETFITEYMDIFESKRDYYVGPGNEKSRTDYGRFTTKIDTETGKYITEDNIFEKLIGKCSVYNDELRASSASFTAQEFNVLNDLNNLTVNGRELEEDEKRKIIKSIFTAKSMRMRKFITEAIGEPIIDMKGARIDGKDKEIFHTFEAYREIKKALNTIDVDINDLSLESMNLSAHILTINTEKDAIIKAFAKTDMHLANEVVECFIKTRKKTPQLFNKWHSLSLKIMNELIDELYKRPVNQMVLLTEMGLFKDNIEKFKGLLKIPVDSVLEELYNPVVRKAVRQSIEVLNAIMKTYGQPTDIIIEMARDKNEEDQKKRIKKIQGDNEKELAKIIKKISLEYGVDIEQKHYHQNKGLKTKLRLWNEQGGVCLYSGKPIVIADLLYDSEKFEIDHVIPISISFDDSRKNKVLVYRSENQTKGNRTPYMYLSTVSREWDFERYAYHISELEKKKLITTYKKRNLLFKENITKQDVVKGFIQRNLNDTRYASRVVLNCVQDYMRANDKPTKVKVIRGSFTSQLRKRLNLPKDRDESYAHHAVDAMIMCYSLMGLDAYKSSQKDVIDYETGEILDQKVYDKLSEEELYNRYMFYMRLMEIKLAIKKGEERIKYSHKVDKKVNRKLSNETIYGVREKIDGTKQKISKINNIYDINGYVNFKNKKDKNKLNDFLMYHHDPKTFEKLLKILEEYADQPNPFLTYKQEIGEPIRKYSKKDNGPFINSLKYYDGEVGSCIDISHKYGHEKGSKKVVLDSLSPYRTDVYYREEDKSYHLVGIKYANFKFMKGEYILDEESYNEILVSEKLLKSGECYTDIEKLGYEFRFSLYKNDIILYEKNGEEKIERFLSRTMPKVKNYIETKPIDANKFSKQNPVGLGKTTNVVKINTDILGNKHFSQKEKFIIKFKLDR

SEQ ID NO: 73 - protein, 1354 aa, synthetic construct
VKERGNYFLGLDLGTSSIGWAVTDQNYRLIRAKGKDLWGVRLFKEAETSESRRTFRVSRRRRQREVARIGLLKEFFSEEISKVDPSFYQRLNESKYHRDEKLVDSRHALFSDKSFTDQDYFSCYPTIFHLRKELILSKEPHDVRLVFLAILNLFKRRGHFLNTNLDAEQSLGFNTLFDKLLYLLSDRFDLRLDTSIVIQNIEETLSDKTYSRTQKAEQIAAELSVKKTSDRMFYELIRAITGLKFTIDVMFGDKYRDSDNNKMSLSFRDNNYDEEVINISEIIGEENMEIINILKLIHDQALLSNVMQGHKYLSEARVESYQKHHEDLKLLKKTIKKFTIEDYDILFRSDEDGSYSAYVGSVNSRKKQRRNMKKRNSEEFYVNLKKILKKCPSTDADVKYILKEIKNENLLPKQLTSDNGVIPNQVHLAELKAILCNASKYLNFLNEKDEKGLTISDKIIGLFSFRIPYYLGPLNNYHSNKGGNAWIVRKSKGQVLPWNIEEMVDEEETSKVFIERLVRKCTYIKDETVLPKSSLLYERFLVLNELNNLKIRGVKPDVELKQEIFNELFLAGKKVSRKKLNSFLVSRGHINPNEIEAITGIDGDFANSLSTFGKFHSVLGDLACEDEGKAIIEQIVFWGTVYGDDKKFYKEKLKRELADKLNAEQIKKIIGFKFRDWGRLSKEFLEMQGVNKSTGEMLPMIQMLWNYNENIMELLSDRYTYTETLINEYSAISKTLSELEYDDLEGLNLSSPVKRMVWQTLLLVKEIEKTLGNPPERIFVEMARERGERGKRTTSRKQRLIDLYKNCKKDTRDWVGELENLEDSILRSKKLYLYYVQNGRCMYTGEPIDLDKLMNDNLYDIDHIYPRRYVKDDSLDNNLVLVKKQTNASKSDQYPIDNSIYIKQHAYWKSLLTKDMTDGFITREKYNRLINRNPLSEEQLAGFINRQIVETRQGTKVITQILKQAFSGSEMVYVKARNISDFRDEFDLIKVRSVNDFHHAQDAYLSCVVGNTYYVKFTKDSLNFVKESRRNNNSKYHMSKIFHYTVKRGEEVAWIAEEQSGDNSIRIVKTMTNKNTPIITKMTLEGHGKLFDATLYSSQKAKGESYVPLKTTDSRIQNVQKYGGYTSVSTAHFFLVEHVEKGKRIRTIEALPLMFRDSIGTNLTKLNDYCKNVLQLKEPDVRLAKIKLQSLIKRDGFYMNITGKTLNRNIVSNAVPLILSRKWLDYIKKVEKTVERDYTDHKITSELNMRLYEELTEKHKNSIYSKRPNPIGKVLKTGQEYFAKLDLMEQCFVLSQILQISQLSNMGANLKLIQGSKKSGISLIPKNISMAEEFLLVNQSITGLYCSEIDLKTI

SEQ ID NO: 74 - protein, 1101 aa, synthetic construct
LGIRFGLDIGIASVGWAVVNDNYEVEEAGSNIFSSADANQNTERRMFRQTKRLHRRRKNRISDFKKLWIDTGLEIPDLNINNILELRIKGLKEKLSQEEIFHVLRNELLHRGISYLDDAIDSSAINQSDYAKGINENQKELDNGCFPCEIQAKRLKKYGKYHGNTSVGTEEDKIILSNVFTTGAYKKEIQAILEQQQQFSSFLTESFCGKYMEIFLRKREYYVGPGNEKSRTDYGIFTTKIDKTTGKYFTDKNIFEKLIGKCSVYKNKLRAAGATYTAQEFNLLNDLNNISINGRKLSKQEKEAVIEEVKTANSFNMRKILKKITGEEIETLTGARLDKNEKEDFHKFAQYNTMRKEFEKNGWDITTLSREELDNIGNILTLNTEKEAIIKEFIHKDLSLTEEMTACLVNLRKQKSSLFNKWQSFSLEIMLELIPGLYEQPKNQMELLTDMGAFKSNKDLFTEYKNIPAEIILENIYNPVVSRSIRVTIQIINALIKKYGYPDQIIIEMPRDKNEDEMKKRISDCQKNNQKELDNIIKRISSEYGIKITDQDFYQHKKLVLKLKLWNEQGGKCLYSGKKIDIHDLLYNPTLFEIDHIIPKSISFDDSRSNKVLVYSTENQKKGNTTPYIYLNSIKKEWGYNEYMAYVLQLKKDKLITKNKMEKLLFTEDITKIQVLKGFISRNINDTRYASRVVLNTLQNYFKAKNAITKIKVVRGSFTHQMRDNLHLDKNREESYSHHAVDAMIMCYSQMGFDNYHKLQTDLIDVETGEIIDIHKWQNEINEKTYNELIYQNKMIHIRANIQKAEKQIKFWHKRDTKPNRGLCNQTIRGTRTENGVIKKVNKLNIYNPSDVETLKKMIKNGKEDSFLMYQHDPRTWENMIQIMQDYRSSKNPFADYENETGGYFRKYSKKHNGPRITKLKYLNGEVNSCIDISHKYGLETGSKKVILESLKPYRMDVYYNEKEQKYYLIGIKYSDLKFSKNKCIINEEAYKKILVYEKLITPEQNRADLEKLGYKFMLSFYKDEIIQYEKNGEYFTERFLSRTMPKQRNYIETKPIDAPQFEKQPRHLVGLGKTTSIKKIRLDILGNRYYCTQEKFQTEC

SEQ ID NO: 75 - protein, 1311 aa, synthetic construct
VKENEEYNIGLDIGTASVGWCVTDLNNNVLKIKGKNMWGSRIFEEAQTAAERRNFRSARRRLTRRKERINILQDLMREDIEKEYSNFLPRLKETSLKYEDKIFSNSKYNLFSDNNRHEISYYEEFPTIYHLRNKLIKEENKVDIRLVYLAIHHIIKYRGNFLYDGDFSNDTSKVDDNLEIILEFLNQNYQIELKSKDDLKNILLSKNLSKAQKKDELMACFNFVSSDKTIITNIINAILGYSFEINKIFDCDLEKSKIKFSNEIDNEEEIENSLESNIDIYYALKDLYSWYILQDIMSNCNYISESFMKRYEKFKEDLKLLKKIYKEYFPNQYDEMFKVKGKNNFVNYIGKNKGEKFGKCDPEDFFKMLKSKIKELPDTCTYKEQIELDLQNDDFLRKLNIIDNGAIPYQLHKNELSIIIKNQSKYYNTLKENGEKIISLLEFRIPYYVGPLANDKFNKWNWVVRNNENEKIRPWNFSEIINEEETAEKFINRMTNKCTYLLNEKVMPKESLLYSKFCVLNELNNIRINEKHIPRDLKKKIVDKLFKQNKKVTIDMFKELCKIEGLPECDIIGIQDGKNFMSNMSSYMDMSKIFGKVDENNYDMCETLIYWITIFEDKKILKKKIKATYSTITEEQLNKLLKLKYSGWSRLSKKLLVGLKSNDYETIMDKLEKTSMNFMQIINNKNFGFDKKINDLAPVIDKNITYKDVSEIPTSPANKRGIWQAICVVKEITKVMNAEPKNIFIEFARSEEFNKNMSQKRAKKLLKIYDNIQDELATLKEYDSNVYEKLKENKSNKDFSERMQLYFMQLGKCLYSGEPLNIEDLNLYEVDHILPQAYIKDDSIDNKALVKRIENQTKKDNLLLSDKIINNMSGYWKKLLDLGLITKEKYLRLTKRKIFENDYEQENFVKRQLVETRQITKYVTNLLKNSYTNTDIFTIRAELTHAFREEFGIYKNRNVNNYHHAHDAYIISIIGNVMNTYIRNNEEFKYGDFVKKYLKENKNKAEKNGMIIGSIKKNIDIGNIKKVFYYKDCFISRMLIEGTGAFYDQTLYSPKINKNKLISLKDDKDSTKYGGYSGEKKAYFSILEYINENGLKEYKLLGIPIKVAYDIKAKKYSEEEYIKKVFFKNNDITELKVIKKKVLKNQQYLDENKEVMRFCSDSEIRVDKELVLNYKMNELIYLMNMKENQLKDNEKDLLNNNFEYMFDYLLEKIKTEYKVFSSIYKKLLDKKEQYMLLENEDKKCVINGLVDLMKNGQGNLKKVGLTEREGRKSKQNFDTKRLTNMIFIDKSVTGIYERRINIYGMENSSSK

SEQ ID NO: 76 - protein, 1364 aa, synthetic construct
MSFCQIFIKVGGKTMKKKYYIGLDMGTSSCGWAVTDENYCLVRAKGKDMWGVRLFSEAQTAAERRTHRIARRRLQREKARIGYLREYFSNEINKVDPGFYQRMDDSKYYPEDKREEQKNTIFADRDYTDQDYYKEYPTIFHLRKELIDSEEKHDIRLIYLAILNMFKHRGHFLNANLGDGEVGNLNELYQSLTEMLSEFMEISLVSLDSTKELEHILVSKEYSNTAKWEQLMEVLNVKKNKNKSEAEMLRLICGLSGRLSKIFVNDAFDEESAKLSISFRDGNFEDKCVTVEQILSEESYEIFLLLKQIHDWGVLAGIMKGYEYLSYARVESYEKHKKDLEILKRVFQKYDKKQYDEMFRCMKKDNYSAYVGSVNSGKESARRTEKTTATDFYKNLEKRVKKLPVDEDTSYILDEISKETFLPKQLTPSNGVIPNQVHKKELIKILKNAEKHYVFLSRKDETGLSVSEKIVQLFEFQIPYYIGPFDNADGKGNGWAVRKENGPVMPWNIEEKIDTKQSAEKFIEKMVNHCTYLNGETVLPKNSLLYEKFMVLNELNNLKINGESISVDLKQQIYTQLFQKGKKVTAKKLINFLKIHNYVDQEDVEVSGIDGDFVNTLSNHKKFCEVFQVDCLSQSQLEMAEKIIFWSTIYGDSKKFLQEKILEEYGEVLDAAQIKRITGYKFKDWGRLSKQFLQLEGADTDTGEVQTIISRMWNENYNLMELLSNHFTYLDEIENKSAKIEKTLQDIMYEDLDELYISAPVKRMVWQTILILKEIYQVMGCAPERIFVEMARDQDAPKKRTISRKNKFAELYKKCKEDGRDWLKEIKNIDESRFRSKKLYLYYTQKGRCMYTGEPIDLEDLFNDNLYDIDHIYPRHFTKDDSIENNLVLVKKEKNAHKSDSFPIENSIRMKQHGWWKTLQDGGFITKEKYKRLVRNTEFTNEELANFVSRQIVETRQGTKVITQLFEKTFPESDIVYAKAGNVSAFRQKFDLLKCRIVNDFHHANDAYLNIVVGNTYFVKFTKNPYQFINGYRKDPSKYRFHMYKIFDYLVRNRNEIAWQTENDQSIRIVKKMMRKNTPLITRMNYEEHGGIADQTIYSAADAKKAKGVGYLPIKASDNKLANTEKYGGYKKFTGTYFFLVEHTVKKKRIRTLEAMPLYKVENLDTIEKLENYCRQELKLDDPSIRMKKIKMYSLIKVNGFYLYLTGRSGNQLLVSNAVPMVLSYDYVKYVKKISQIQERDHSLEEIEEMEEISSKKNLELYNQLTYKYSKAIYAQRPNPIGDKLSNWKEKFISLNIKDQINVLLQILQLSMLSNMGADLTAIGGVKKTGVATLNKKISDKDEFKLINQSVTGLYESEIDLLKV

SEQ ID NO: 77 - protein, 1343 aa, synthetic construct
MAKRYNIGLDIGTSSVGWAVVEADTQKIIKKGNKKTALWGVRLFDGAQTAESRRQSRSTRRRYDRRRQRIQLLQEEFKNEINKVDVNFYTKLKESKYNKSDKINKTIIISKDEQKLINEYNKKYPTIYHLRNKLVISDEKEDIRLVYLAMHHIIKYRGNFNYQGDHFNVNDLNIKDKLIFLLTSLSEQYPELEIDEEYSSKITDDIIQNSLLNPSKNDMKINTKELLLELTSNSKFVSEFIKLLNGNKFSIKKLFGIEELEEDIQISFSNSDFDDNYEKLEQTVPDKIETLNIMKELYDMIFLKKLFVGSDSTNLSALMVERYNQHKEDLKFLKSIFNIDRKIYNKIFRDGKEKCLYTKYITNEISNDEFIKEIEKYLPKKYEGSNEKIRMDNGIFLPRITSTENGKYPYQLNEDELIKIIEKQGKYYKFLLDKVDDKYKIVKLLEFKIPYYVGPLNKNSQFAWLEKTINEKITPYNFDKIVDKESTAEKFIKRMISHCTYLDDKVDNNGNIIEENYALPNCSILYSEFKVRNELKQIKINGDSIKNENQHKIFDELFKKTNGTITEKKFKDYLKVCPDYYPFEGNFNITGYSADGRFANNMQSYVDFFGENGIFDGTDYTLDDAEKIIEWITIFEDKDILKSKVLNNYDKLSEEAVKKILNKKYSGWGSLSRRLLCGIVTKDKKSGIGKTIMDYMMETKENFMQILNNDEYDFQGKISEYNKVNNHVKLNYSLVENLATSPSTKRGIYQALKVVDELTKYIGYAPENIMVEMAREDEKKVRKDDRKTYLEKLYKENKIEIDNYNRLYSELESRDKIDSEKLYLYFIQEGKCLYTGEPINIDELNNDEYEVDHIIPRTLIKDDSIDNKALVLRRCNQDKKDSFVLPKEYRTERMLDWWHHLKKMKLMTAKKFYSLKRKEYTDDDIQGFINRQLVETRQITKHVADILGSLYSKTKIVYLKANISHNYRERYELFKFREINDYHHAHDAYLAAVLGEYKEKYMKCKIDFESVKELNKKFYENGDYKKLKYGFVINCLDENEFATISKIIKNFVDDKTGEVLFDVSKFNRTIENTLYRNDILISKKTEIRTGEFYNQTKKKKCNDGIKLKGNMPTAMYGAYTSVNPSYAVMVEYDYKKCKNRKMVGMPIYLKTDAEKVSYLKMKLGLNEGDSLDIISKPIPFYAYLNWDNQLCYLVGASDKVEVVNAKEFNYNKEFYIKHKYTLHKVFHNKNYEIEDSKYSQELSEIIKYIVDKIENEYELFKNLVPELKSIVKYNEKIDFSVENKEKIIIQLTKLLNCTSDNANFKFLNDSYSSAFGKKHGRTIEHCLFVSKSVTSIYEATNEF

SEQ ID NO: 78 - protein, 1187 aa, synthetic construct
MKDDNEKAKFILGIDVGISSVGWAIVDLDELDSPYKIKDLGVRIFTAAEVPKTGDSKAKSRREKRGIRRLIRRKAFRMDRVRYLLNQNGFLGEKITDGKVDIINHNLEKEAAKIIENYNIGKNIMPYELKVKGLTEKLLKEELLIIMLHYAKHRGYKSNREDNEAAKESTSESGKTLSAINDNRKIMIENKYEIVSELFLNDDKFKGRIRNTNNDYKMCVTREMYLEEINKVLDKQISLEVITSEFKDEFIKIWSDQRHYSKGPGGNSKYGGDLIERMVGKCQFTGEPRAPKCAPSSEVFVSVSKLVNLRYKQPDDKEYKSLTENEITIAIELAKEQDKVTYKKLIKELKLPDNTIIKGLELSKKDFIEFIVKFKKEVLNREDKEKVDKATLTEEESNEYDKRYFDKLNSKTCLELKSYSIIRKAFIKQLGTEKWDNIKNDFDLLDEFARLLTYYKLNEEIKRELDKIENLRDEYKDVVISLPTFKDHLSISLSLVKSLIPLISSGMRYDEAMNELGYNHSDLNGDIEKYDLLVSLNKGTSINNQRVMRSLAQTRKVVNAVIKKYGIPYQINVETARELAKSRQERNEIEKKQRENQENNEKIKNDLVDKFPNIFSDTSKVSGVDIQKYKLWIEQNETCAYSLERITIEKLFDKNEVQIDHILPYSRTMNDNYSNRTLVLTKANQEKGNRTPYEWFGKTDKWANFENFINNINISTSKKDNYLLINLTQEVEQEMRDQNLNDTKYISRHLSTMIRKYLNVEKFQMVSGSITGRLRARWGLNNLTHSLVSPTYYQSGKNKDREHHLHHAMDALVIAVTTPSLIQKVSHYEKFRRYIVGKTETEIRNATSDEVFTFYETGEVLTFREYLNKTIEEKYLFQNKKGTMDVLFPLPYDKFITEAKLRVYERNEEVLKRELLELKTYTREEIDNATVIIPSFMKIKPSGAYHAETFYGIKKVEDTTYKIEKKKVTSLKKKDLEDIVEKNGGSKEVYNTLVDWFEDNEEGSEVFKQKGYPVNKKSGNLIKAVRLQEEYKGKGHIINGAVVEKDNIYQIEIYKKDGSDKLYFIGLDNLEINKIKNGKEFNVTLWYGRDNNKIDNISYKKVNEEYTKVASLKKNDLIEIELNSGVKGKCCANGFSGGKFEISSLIGDGYDLVSSKLFGNLIERYQIAISTIKSIEILKISVLGKIE

SEQ ID NO: 79-122 - skipped (44 entries)

SEQ ID NO: 123 - RNA, 16 nt, synthetic construct
  source 1..16: mol_type = other RNA
gtttgagagacttgta

SEQ ID NO: 124 - RNA, 17 nt, synthetic construct
  source 1..17: mol_type = other RNA
atttaagtacctggaga

SEQ ID NO: 125 - RNA, 17 nt, synthetic construct
  source 1..17: mol_type = other RNA
gtttaagatctatgtca

SEQ ID NO: 126 - RNA, 16 nt, synthetic construct
  source 1..16: mol_type = other RNA
gtttgagagtcttgtt

SEQ ID NO: 127 - RNA, 17 nt, synthetic construct
  source 1..17: mol_type = other RNA
gttttagctctatgtta

SEQ ID NO: 128 - RNA, 16 nt, synthetic construct
  source 1..16: mol_type = other RNA
gtttagttaccatatg

SEQ ID NO: 129 - RNA, 17 nt, synthetic construct
  source 1..17: mol_type = other RNA
gttttgttaccatatgg

SEQ ID NO: 130 - RNA, 17 nt, synthetic construct
  source 1..17: mol_type = other RNA
gttttagaagtatgtta

SEQ ID NO: 131 - RNA, 17 nt, synthetic construct
  source 1..17: mol_type = other RNA
gtttaagaagtatgtta

SEQ ID NO: 132 - RNA, 17 nt, synthetic construct
  source 1..17: mol_type = other RNA
gttttagaattatgtta

SEQ ID NO: 133 - RNA, 17 nt, synthetic construct
  source 1..17: mol_type = other RNA
gtttaagaattatgtta

SEQ ID NO: 134 - RNA, 16 nt, synthetic construct
  source 1..16: mol_type = other RNA
gtttgagagtattgta

SEQ ID NO: 135 - RNA, 17 nt, synthetic construct
  source 1..17: mol_type = other RNA
atttaagtacctagtag

SEQ ID NO: 136 - RNA, 16 nt, synthetic construct
  source 1..16: mol_type = other RNA
gtttgagagccctgta

SEQ ID NO: 137 - RNA, 16 nt, synthetic construct
  source 1..16: mol_type = other RNA
gtttgagaaccttgta

SEQ ID NO: 138 - RNA, 17 nt, synthetic construct
  source 1..17: mol_type = other RNA
gtttaagtacctagtaa

SEQ ID NO: 139 - RNA, 16 nt, synthetic construct
  source 1..16: mol_type = other RNA
gtttgagaaccctgta

SEQ ID NO: 140 - RNA, 16 nt, synthetic construct
  source 1..16: mol_type = other RNA
gtttgagagacttgtt

SEQ ID NO: 141 - RNA, 16 nt, synthetic construct
  source 1..16: mol_type = other RNA
gtttgagagccttgtt

SEQ ID NO: 142 - RNA, 16 nt, synthetic construct
  source 1..16: mol_type = other RNA
gtttgagagctttgtt

SEQ ID NO: 143 - RNA, 17 nt, synthetic construct
  source 1..17: mol_type = other RNA
atttaagtacccggtga

SEQ ID NO: 144 - RNA, 17 nt, synthetic construct
  source 1..17: mol_type = other RNA
gtttagttaccataagg

SEQ ID NO: 145 - RNA, 17 nt, synthetic construct
  source 1..17: mol_type = other RNA
gttttgttaccataagg

SEQ ID NO: 146 - RNA, 17 nt, synthetic construct
  source 1..17: mol_type = other RNA
gttttagtattgtgtta

SEQ ID NO: 147 - RNA, 17 nt, synthetic construct
  source 1..17: mol_type = other RNA
atttaagtacctgtaag

SEQ ID NO: 148 - RNA, 17 nt, synthetic construct
  source 1..17: mol_type = other RNA
gttttagaattgtgtta

SEQ ID NO: 149 - RNA, 17 nt, synthetic construct
  source 1..17: mol_type = other RNA
gtttaagaattgtgtta

SEQ ID NO: 150 - RNA, 17 nt, synthetic construct
  source 1..17: mol_type = other RNA
gtttaagttctatgtca

SEQ ID NO: 151 - RNA, 17 nt, synthetic construct
  source 1..17: mol_type = other RNA
gtttagctaccatctat

SEQ ID NO: 152 - RNA, 17 nt, synthetic construct
  source 1..17: mol_type = other RNA
gtttaagtattgtgtta

SEQ ID NO: 153 - RNA, 17 nt, synthetic construct
  source 1..17: mol_type = other RNA
gttttagaaatatgtta

SEQ ID NO: 154 - RNA, 17 nt, synthetic construct
  source 1..17: mol_type = other RNA
gtttaagaaatatgtta

SEQ ID NO: 155 - RNA, 17 nt, synthetic construct
  source 1..17: mol_type = other RNA
gtttaagtacctggtag

SEQ ID NO: 156 - RNA, 17 nt, synthetic construct
  source 1..17: mol_type = other RNA
gtttagctaccatatgg

SEQ ID NO: 157 - RNA, 17 nt, synthetic construct
  source 1..17: mol_type = other RNA
gttttagagttgtgtta

SEQ ID NO: 158 - RNA, 17 nt, synthetic construct
  source 1..17: mol_type = other RNA
gtttagttaccataaag

SEQ ID NO: 159 - RNA, 17 nt, synthetic construct
  source 1..17: mol_type = other RNA
atttaagtacctgtaga

SEQ ID NO: 160 - RNA, 17 nt, synthetic construct
  source 1..17: mol_type = other RNA
attttagtacctgaaga

SEQ ID NO: 161 - RNA, 17 nt, synthetic construct
  source 1..17: mol_type = other RNA
gttttagatttatgtta

SEQ ID NO: 162 - RNA, 17 nt, synthetic construct
  source 1..17: mol_type = other RNA
gtttaagatttatgtta

SEQ ID NO: 163 - RNA, 17 nt, synthetic construct
  source 1..17: mol_type = other RNA
atttaagtacctgtaaa

SEQ ID NO: 164 - RNA, 17 nt, synthetic construct
  source 1..17: mol_type = other RNA
gttttattaccattaat

SEQ ID NO: 165 - RNA, 17 nt, synthetic construct
  source 1..17: mol_type = other RNA
gtttagttaccatatga

SEQ ID NO: 166 - RNA, 103 nt, synthetic construct
  source 1..103: mol_type = other RNA
gtttgagagacttgtagaaatacaagacgagtgcaaataaggatttatccgaaatcgtcgcaagactctacatggtgtagaacttaaaagacttggtttcaag

SEQ ID NO: 167 - RNA, 61 nt, synthetic construct
  source 1..61: mol_type = other RNA
atttaagtacctggagagaaattatcttcagacctacttaaacaaggctttatgccgaaag

SEQ ID NO: 168 - RNA, 83 nt, synthetic construct
  source 1..83: mol_type = other RNA
gtttaagatctatgtcagaaatgacatagagagtttaaataaagctttaagcctaaatgctcacgtatgtgttcgaacagtta

SEQ ID NO: 169 - RNA, 103 nt, synthetic construct
  source 1..103: mol_type = other RNA
gtttgagagtcttgttgaaaaacaagacgagtgcaaataaggtttaaccgtaatcgtcgaaagactctgcatggtgcagaaagaaagaggctgtataaagcct

SEQ ID NO: 170 - RNA, 83 nt, synthetic construct
  source 1..83: mol_type = other RNA
gtttaagatctatgtcagaaatgacatagagagtttaaataaagctttaagcctaaatgctcacatatgtgttcagacggtta

SEQ ID NO: 171 - RNA, 105 nt, synthetic construct
  source 1..105: mol_type = other RNA
gttttagctctatgttagaaatgacatagataagttaaaataaaagtttcctatatggatactctaaacacttactattgtaggttgctgcgtgtgcagctcttt

SEQ ID NO: 172 - RNA, 83 nt, synthetic construct
  source 1..83: mol_type = other RNA
gtttaagatctatgtcagaaatgacatagaaaagtttaaataaaggcttagccctaaatactcctttggagaacgactgtgta

SEQ ID NO: 173 - RNA, 83 nt, synthetic construct
  source 1..83: mol_type = other RNA
gtttaagatctatgtcagaaatgacatagagagtttaaataaaggttttaccctaaatactcttctgagttgctgcttaggca

SEQ ID NO: 174 - RNA, 73 nt, synthetic construct
  source 1..73: mol_type = other RNA
gtttagttaccatatgggaaactatatgacctaactaaacaagggtttatcccggattcggctcttcggagcc

SEQ ID NO: 175 - RNA, 73 nt, synthetic construct
  source 1..73: mol_type = other RNA
gttttgttaccatatgggaaactatatgacctaacaaaacaagggtttatcccggattcggctcttcggagcc

SEQ ID NO: 176 - RNA, 83 nt, synthetic construct
  source 1..83: mol_type = other RNA
gttttagaagtatgttagaaataacataccgagttaaaataaggtttaaccttaaatactggctatgccagattcatttagat

SEQ ID NO: 177 - RNA, 111 nt, synthetic construct
  source 1..111: mol_type = other RNA
gtttaagaagtatgttagaaataacataccgagtttaaataaggtttaaccttaaatactggctatgccagattcatttagatgaagtagcgcctctttggaggtgcttta

SEQ ID NO: 178 - RNA, 83 nt, synthetic construct
  source 1..83: mol_type = other RNA
gttttagaattatgttagaaatagcataatgagttaaaataaggttaaacctttaatgctgatttatatcagtttcgcttagg

SEQ ID NO: 179 - RNA, 109 nt, synthetic construct
  source 1..109: mol_type = other RNA
gtttaagaattatgttagaaatagcataatgagtttaaataaggttaaacctttaatgctgatttatatcagtttcgcttaggtgaaatgagctctttgagctcttttt

SEQ ID NO: 180 - RNA, 83 nt, synthetic construct
  source 1..83: mol_type = other RNA
gtttaagatctatgtcagaaatgacatagagagtttaaataaagctttaagcctaaatgctcacttgtgttcggacggttagc

SEQ ID NO: 181 - RNA, 103 nt, synthetic construct
  source 1..103: mol_type = other RNA
gtttgagagtattgtagaaatacaagacgagtgcaaataaggtttatccgaaatcgtcgtatgactctacatggtgtagataataaaaaggacttgcgtgcaa

SEQ ID NO: 182 - RNA, 103 nt, synthetic construct
  source 1..103: mol_type = other RNA
gtttgagagacttgtagaaatacaagacgagtgcaaataaggatttatccgaaatcgtcgcaagactctacatggtgtagaacttaaaaggcttggtttcaag

SEQ ID NO: 183 - RNA, 78 nt, synthetic construct
  source 1..78: mol_type = other RNA
atttaagtacctagtaggaaatattagatctacttaaacaaggctttatgccgaaatcaaggacaccgacgggtgtcc

SEQ ID NO: 184 - RNA, 103 nt, synthetic construct
  source 1..103: mol_type = other RNA
gtttgagagccctgtagaaatacagggtgagttcaaataaggctttagccgaaatcgcgcctcgtgtccgcattgtgcgggtttaaaagagaatcgtaagatt

SEQ ID NO: 185 - RNA, 103 nt, synthetic construct
  source 1..103: mol_type = other RNA
gtttgagaaccttgtagaaatacaaggtgagttcaaataaggagcaatccgaaattacattgaccgcattgtgcggatatgaaagacgtagcctatggctaag

SEQ ID NO: 186 - RNA, 103 nt, synthetic construct
  source 1..103: mol_type = other RNA
gtttgagaaccttgtagaaaacaaggtgagttcaaataaggatttatccgaaattacatttgcccgcattgtgcggattgaaaaggttcgacttatgtcgaat

SEQ ID NO: 187 - RNA, 81 nt, synthetic construct
  source 1..81: mol_type = other RNA
gtttaagtacctagtaagaaattattagacctacttaaataaggctttatgccgaaatcaagaacacccgacggggtgttc

SEQ ID NO: 188 - RNA, 103 nt, synthetic construct
  source 1..103: mol_type = other RNA
gtttgagaaccctgtagaaatacagggtgagttcaaataaggcattagccgaaattgcacctgatgtccgcattgtgcgggattaaagaggatcgaaggattc

SEQ ID NO: 189 - RNA, 103 nt, synthetic construct
  source 1..103: mol_type = other RNA
gtttgagagccctgtagaaatacagggtgagttcaaataaggctttagccgatatcgcacctcgtgtccgcattgtgcgggattttaagagaatcgtttcgat

SEQ ID NO: 190 - RNA, 103 nt, synthetic construct
  source 1..103: mol_type = other RNA
gtttgagagacttgttgaaaaacaggtcgagtgcaaataaggtttatccgaactcgtcaatttgatccgcattgtgcggtttttaaaatccgatgtttcatcg

SEQ ID NO: 191 - RNA, 103 nt, synthetic construct
  source 1..103: mol_type = other RNA
gtttgagagacttgttgaaaaacaggtcgagtgcaaataaggtttatccgaactcgtcaatttgatccgcattgtgcggttattaaaatccgatgtttcatcg

SEQ ID NO: 192 - RNA, 103 nt, synthetic construct
  source 1..103: mol_type = other RNA
gtttgagagccttgttgaaaaacaaggcgagttcaaataaggatttttccgaaatcgctatttagtctgcagtgtgcagtttcgtgtccggctttgccgggca

SEQ ID NO: 193 - RNA, 103 nt, synthetic construct
  source 1..103: mol_type = other RNA
gtttgagagccttgttgaaaaacaaggcgagttcaaataaggattattccgaaatcgctatttagtctgcagtgtgcagtttcgtgtccggctttgccgggca

SEQ ID NO: 194 - RNA, 103 nt, synthetic construct
  source 1..103: mol_type = other RNA
gtttgagagctttgttgaaaaacaaagcgagtgcaaataaggtttatccgaaatcgcttttatagcaagcattgtgcttattaaatctggctttccagccaga

SEQ ID NO: 195 - RNA, 103 nt, synthetic construct
  source 1..103: mol_type = other RNA
gtttgagagccctgtagaaatacagggtgagttcaaataaggctttagccgaaatcgcacctcgtgtccgcattgtgcgggtttaaagagaatcgtttgattc

SEQ ID NO: 196 - RNA, 103 nt, synthetic construct
  source 1..103: mol_type = other RNA
gtttgagagccctgtagaaatacagggtgagttcaaataaggctttagccgaaatcgcacctgagtgtccgcattgtgcgggattaagagaatcgaaagattc

SEQ ID NO: 197 - RNA, 103 nt, synthetic construct
  source 1..103: mol_type = other RNA
gtttgagaaccttgtagaaatacaaggtgagttcaaataaggatttatccgacatcgccgtaaggcccacattgtgtggtttaaaagacttgagaaagtcttt

SEQ ID NO: 198 - RNA, 61 nt, synthetic construct
  source 1..61: mol_type = other RNA
atttaagtacccggtgagaaaatttcttcggacctatttaaacaaggctttatgccgaaag

SEQ ID NO: 199 - RNA, 73 nt, synthetic construct
  source 1..73: mol_type = other RNA
gtttagttaccataagggaaattttatgacctaactaaacaagggtttatcccggattcggctcttcggagcc

SEQ ID NO: 200 - RNA, 73 nt, synthetic construct
  source 1..73: mol_type = other RNA
gttttgttaccataagggaaattttatgacctaacaaaacaagggtttatcccggattcggctcttcggagcc

SEQ ID NO: 201 - RNA, 83 nt, synthetic construct
  source 1..83: mol_type = other RNA
gttttagtattgtgttagaaatagcacaatgagttaaaataaggtttaaaccttaaatgccaacttgatgttggtttcactta

SEQ ID NO: 202 - RNA, 81 nt, synthetic construct
  source 1..81: mol_type = other RNA
atttaagtacctgtaaggaaatttacagatctacttaaataaggctttatgccgaaatcaaggagtgccgacgggcactcc

SEQ ID NO: 203 - RNA, 83 nt, synthetic construct
  source 1..83: mol_type = other RNA
gttttagaattgtgttagaaatagcacaatgagttaaaataagggtaaccttaaatgccaattcatattggtttcacttaggt

SEQ ID NO: 204 - RNA, 114 nt, synthetic construct
  source 1..114: mol_type = other RNA
gtttaagaattgtgttagaaatagcacaatgagtttaaataagggtaaccttaaatgccaattcatattggtttcacttaggtgaataaagagttctagcttgctagggctttt

SEQ ID NO: 205 - RNA, 103 nt, synthetic construct
  source 1..103: mol_type = other RNA
gtttgagaaccttgtagaaatacaaggtgagtgcaaataaggatttatccgacatcgccgtttggtctgcatggtgcaggtaaaagacttggtttcaagtctt

SEQ ID NO: 206 - RNA, 83 nt, synthetic construct
  source 1..83: mol_type = other RNA
gtttaagttctatgtcagaaatgacatagagagtttaaataaaggttttaccctaaatgctcacgtatgtgttcggctgctta

SEQ ID NO: 207 - RNA, 81 nt, synthetic construct
  source 1..81: mol_type = other RNA
atttaagtacctgtaaggaaatttacagatctacttaaataaggctttatgccgagattaaggagtgccgacgggtactcc

SEQ ID NO: 208 - RNA, 77 nt, synthetic construct
  source 1..77: mol_type = other RNA
gtttagctaccatctatgaaagatatatgatctagctaaacaagggtttatcccggattcggctcctataaggagcc

SEQ ID NO: 209 - RNA, 114 nt, synthetic construct
  source 1..114: mol_type = other RNA
gtttaagtattgtgttagaaatagcacaatgagtttaaataaggtttaaccttaaatgccaatttatgttggtttcacttaggtgaatttaagttctagttatctagagctttt

SEQ ID NO: 210 - RNA, 83 nt, synthetic construct
  source 1..83: mol_type = other RNA
gttttagaaatatgttagaaatagcatattgagttaaaataagtgtaaactataatgccaagtaattggtttcacttaggtga

SEQ ID NO: 211 - RNA, 108 nt, synthetic construct
  source 1..108: mol_type = other RNA
gtttaagaaatatgttagaaatagcatattgagtttaaataagtgtaaactataatgccaagtaattggtttcacttaggtgaagttagagcttttcgaagctctttt

SEQ ID NO: 212 - RNA, 79 nt, synthetic construct
  source 1..79: mol_type = other RNA
gtttaagtacctggtaggaaattatcagatctacttaaacaaggctttatgccgaaatcaaggacatcgacggatgtcc

SEQ ID NO: 213 - RNA, 103 nt, synthetic construct
  source 1..103: mol_type = other RNA
gtttgagaaccttgtagaaatacaaggtgagttcaaataaggatttatccgaaattgcattttgcccacattgtgtggatttaaaaagaccgttagggtcttt

SEQ ID NO: 214 - RNA, 83 nt, synthetic construct
  source 1..83: mol_type = other RNA
gtttaagatctatgtcagaaatgacatagagagtttaaataaaggcttagccctaaaaatttacacttatgtgggtggttgtt

SEQ ID NO: 215 - RNA, 76 nt, synthetic construct
  source 1..76: mol_type = other RNA
gtttagctaccatatgggaaattatatgacctggctaaacaagggtttatcccggattcggctcctataaggagcc

SEQ ID NO: 216 - RNA, 83 nt, synthetic construct
  source 1..83: mol_type = other RNA
gttttagagttgtgttagaaatagcacaacgagttaaaataaggtttaaccttaaatgtcaattaaatattgattttcactta

SEQ ID NO: 217 - RNA, 73 nt, synthetic construct
  source 1..73: mol_type = other RNA
gtttagttaccataaaggaaattttatgacctaactaaacaagggtttatcccggattcggctcttcggagcc

SEQ ID NO: 218 - RNA, 79 nt, synthetic construct
  source 1..79: mol_type = other RNA
atttaagtacctgtagagaaatttacagatctacttaaacaaggctttatgccgaactcaaggacatcgaaagatgtcc

SEQ ID NO: 219 - RNA, 79 nt, synthetic construct
  source 1..79: mol_type = other RNA
attttagtacctgaagagaaattttcagatctactaaaacaaggctttatgccgaactcaaggacatcgaaagatgtcc

SEQ ID NO: 220 - RNA, 103 nt, synthetic construct
  source 1..103: mol_type = other RNA
gtttgagaaccttgtagaaaacaagtgtgagtgcaaataaggattttccgaaatcgccgaaaggcctgcatggtgcagattaaaagacttgacttttcaagtc

SEQ ID NO: 221 - RNA, 83 nt, synthetic construct
  source 1..83: mol_type = other RNA
gttttagatttatgttagaaatagcataaatgagttaaaataaggtaaaaccttaaatgccaatttaatattggttttgctta

SEQ ID NO: 222 - RNA, 115 nt, synthetic construct
  source 1..115: mol_type = other RNA
gtttaagatttatgttagaaatagcataaatgagtttaaataaggtaaaaccttaaatgccaatttaatattggttttgcttaggcaagtaaagtcttagttatctaaggctttt

SEQ ID NO: 223 - RNA, 103 nt, synthetic construct
  source 1..103: mol_type = other RNA
gtttgagaaccttgtagaaatgcaaggtgagttcaaataaggatttatccgaaatcgccgtacggcccacattgtgtggatgaaaagacttggtttccaggtc

SEQ ID NO: 224 - RNA, 79 nt, synthetic construct
  source 1..79: mol_type = other RNA
atttaagtacctgtaaggaaatttacagatctacttaaacaaggctttatgccgaaatcaaggacatcgacggatgtcc

SEQ ID NO: 225 - RNA, 80 nt, synthetic construct
  source 1..80: mol_type = other RNA
atttaagtacctgtaaagaaatttacagaatctacttaaacaaggctttatgccgaaatcaaggacatcgacggatgtcc

SEQ ID NO: 226 - RNA, 73 nt, synthetic construct
  source 1..73: mol_type = other RNA
gttttattaccattaatgaaattatatgacctaataaaacaagggtttatcccggattcggctcttcggagcc

SEQ ID NO: 227 - RNA, 73 nt, synthetic construct
  source 1..73: mol_type = other RNA
gtttagttaccatatgagaaattatatgacctaactaaacaagggtttatcccggattcggctcttcggagcc

SEQ ID NO: 228 - DNA, 123 bp, synthetic construct
  source 1..123: mol_type = other DNA
tattaaatactcgtattgctgtttgagagacttgtagaaatacaagacgagtgcaaataaggatttatccgaaatcgtcgcaagactctacatggtgtagaacttaaaagacttggtttcaag

SEQ ID NO: 229 - DNA, 81 bp, synthetic construct
  source 1..81: mol_type = other DNA
tattaaatactcgtattgctatttaagtacctggagagaaattatcttcagacctacttaaacaaggctttatgccgaaag

SEQ ID NO: 230 - DNA, 103 bp, synthetic construct
  source 1..103: mol_type = other DNA
tattaaatactcgtattgctgttttagaattgtgttagaaatagcacaatgagttaaaataaggttaaaccttaaatgccagttttctggtttcacttaggtg

SEQ ID NO: 231 - DNA, 131 bp, synthetic construct
  source 1..131: mol_type = other DNA
tattaaatactcgtattgctgtttaagaattgtgttagaaatagcacaatgagtttaaataaggttaaaccttaaatgccagttttctggtttcacttaggtgaattaagttctgtgtaaacagaactttt

SEQ ID NO: 232 - DNA, 103 bp, synthetic construct
  source 1..103: mol_type = other DNA
tattaaatactcgtattgctgtttaagatctatgtcagaaatgacatagagagtttaaataaaggtttaaccctaaatactctatttgagcgactgctttggc

SEQ ID NO: 233 - DNA, 103 bp, synthetic construct
  source 1..103: mol_type = other DNA
tattaaatactcgtattgctgtttaagatctatgtcagaaatgacatagagagtttaaataaagctttaagcctaaatgctcacgtatgtgttcgaacagtta

SEQ ID NO: 234 - DNA, 99 bp, synthetic construct
  source 1..99: mol_type = other DNA
tattaaatactcgtattgctatttaagtacctgtaaggaaatttacagatctacttaaacaaggctttatgccgaaatcaaggacatcgacggatgtcc

SEQ ID NO: 235 - DNA, 100 bp, synthetic construct
  source 1..100: mol_type = other DNA
tattaaatactcgtattgctatttaagtacctgtaaagaaatttacagaatctacttaaacaaggctttatgccgaaatcaaggacatcgacggatgtcc

SEQ ID NO: 236 - DNA, 93 bp, synthetic construct
  source 1..93: mol_type = other DNA
tattaaatactcgtattgctgtttagttaccatatgggaaactatatgacctagctaaacaagggtttatcccggattcggctcttcggagcc

SEQ ID NO: 237 - DNA, 123 bp, synthetic construct
  source 1..123: mol_type = other DNA
tattaaatactcgtattgctgtttgagagtcttgttgaaaaacaagacgagtgcaaataaggtttaaccgtaatcgtcgaaagactctgcatggtgcagaaagaaagaggctgtataaagcct

SEQ ID NO: 238 - DNA, 103 bp, synthetic construct
  source 1..103: mol_type = other DNA
tattaaatactcgtattgctgtttaagatctatgtcagaaatgacatagagagtttaaataaagctttaagcctaaatgctcacatatgtgttcagacggtta

SEQ ID NO: 239 - DNA, 103 bp, synthetic construct
  source 1..103: mol_type = other DNA
tattaaatactcgtattgctgtttaagctctatgttagaaatgacatagataagtttaaataaaagtttcctatatggatactctaaacacttactattgtag

SEQ ID NO: 240 - DNA, 125 bp, synthetic construct
  source 1..125: mol_type = other DNA
tattaaatactcgtattgctgttttagctctatgttagaaatgacatagataagttaaaataaaagtttcctatatggatactctaaacacttactattgtaggttgctgcgtgtgcagctcttt

SEQ ID NO: 241 - DNA, 101 bp, synthetic construct
  source 1..101: mol_type = other DNA
tattaaatactcgtattgctgttacagtaccctgtgagaaattacaggttactgtaataaggcataatgccttaagtgtttgtcgaccactacttgtcgac

SEQ ID NO: 242 - DNA, 103 bp, synthetic construct
  source 1..103: mol_type = other DNA
tattaaatactcgtattgctgtttaagatctatgtcagaaatgacatagagagtttaaataaagctttaagccttaatactcacttagtgagggacggatagc

SEQ ID NO: 243 - DNA, 103 bp, synthetic construct
  source 1..103: mol_type = other DNA
tattaaatactcgtattgctgtttaagatctatgtcagaaatgacatagaaaagtttaaataaaggcttagccctaaatactcctttggagaacgactgtgta

SEQ ID NO: 244 - DNA, 103 bp, synthetic construct
  source 1..103: mol_type = other DNA
tattaaatactcgtattgctgtttaagatctatgtcagaaatgacatagagagtttaaataaaggttttaccctaaatactcttctgagttgctgcttaggca

SEQ ID NO: 245 - DNA, 93 bp, synthetic construct
  source 1..93: mol_type = other DNA
tattaaatactcgtattgctgtttagttaccatatgggaaactatatgacctaactaaacaagggtttatcccggattcggctcttcggagcc

SEQ ID NO: 246 - DNA, 93 bp, synthetic construct
  source 1..93: mol_type = other DNA
tattaaatactcgtattgctgttttgttaccatatgggaaactatatgacctaacaaaacaagggtttatcccggattcggctcttcggagcc

SEQ ID NO: 247 - DNA, 103 bp, synthetic construct
  source 1..103: mol_type = other DNA
tattaaatactcgtattgctgttttagaagtatgttagaaataacataccgagttaaaataaggtttaaccttaaatactggctatgccagattcatttagat

SEQ ID NO: 248 - DNA, 131 bp, synthetic construct
  source 1..131: mol_type = other DNA
tattaaatactcgtattgctgtttaagaagtatgttagaaataacataccgagtttaaataaggtttaaccttaaatactggctatgccagattcatttagatgaagtagcgcctctttggaggtgcttta

SEQ ID NO: 249 - DNA, 97 bp, synthetic construct
  source 1..97: mol_type = other DNA
tattaaatactcgtattgctgttacaggaccctgtatgaaaatacaggtttctgtaataaggcataatgccttaggcttaatcccccttaaggattt

SEQ ID NO: 250 - DNA, 103 bp, synthetic construct
  source 1..103: mol_type = other DNA
tattaaatactcgtattgctgtttaagttctatgtcagaaatgacatagagagtttaaataaaggattacctaattactcattatgtgatgagaggctgctta

SEQ ID NO: 251 - DNA, 115 bp, synthetic construct
  source 1..115: mol_type = other DNA
tattaaatactcgtattgctgttttagttctatgtcagaaatgacatagagagttaaaataaaggattacctaattactcattatgtgatgagaggctgcttaggctggcctttt

SEQ ID NO: 252 - DNA, 103 bp, synthetic construct
  source 1..103: mol_type = other DNA
tattaaatactcgtattgctgttttagaattatgttagaaatagcataatgagttaaaataaggttaaacctttaatgctgatttatatcagtttcgcttagg

SEQ ID NO: 253 - DNA, 129 bp, synthetic construct
  source 1..129: mol_type = other DNA
tattaaatactcgtattgctgtttaagaattatgttagaaatagcataatgagtttaaataaggttaaacctttaatgctgatttatatcagtttcgcttaggtgaaatgagctctttgagctcttttt

SEQ ID NO: 254 - DNA, 103 bp, synthetic construct
  source 1..103: mol_type = other DNA
tattaaatactcgtattgctgtttaagatctatgtcagaaatgacatagagagtttaaataaagctttaagcctaaatgctcacttgtgttcggacggttagc

SEQ ID NO: 255 - DNA, 123 bp, synthetic construct
  source 1..123: mol_type = other DNA
tattaaatactcgtattgctgtttgagagtattgtagaaatacaagacgagtgcaaataaggtttatccgaaatcgtcgtatgactctacatggtgtagataataaaaaggacttgcgtgcaa

SEQ ID NO: 256 - DNA, 123 bp, synthetic construct
  source 1..123: mol_type = other DNA
tattaaatactcgtattgctgtttgagagacttgtagaaatacaagacgagtgcaaataaggatttatccgaaatcgtcgcaagactctacatggtgtagaacttaaaaggcttggtttcaag

SEQ ID NO: 257 - DNA, 123 bp, synthetic construct
  source 1..123: mol_type = other DNA
tattaaatactcgtattgctgtttgagaaccttgtagaaatacaaggtgagttcaaataaggattttatccgacatcgccgtaaggttagcattgtgcgacagaaaagacttcttcggaagtc

SEQ ID NO: 258 - DNA, 93 bp, synthetic construct
  source 1..93: mol_type = other DNA
tattaaatactcgtattgctgtttaattaccattaatgaaattatatgacctaattaaacaagggtttatcccggattcggctcttcggagcc

SEQ ID NO: 259 - DNA, 93 bp, synthetic construct
  source 1..93: mol_type = other DNA
tattaaatactcgtattgctgttttattaccattaatgaaattatatgacctaataaaacaagggtttatcccggattcggctcttcggagcc

SEQ ID NO: 260 - DNA, 103 bp, synthetic construct
  source 1..103: mol_type = other DNA
tattaaatactcgtattgctgttttagaagtatgttagaaataacatactgagttaaaataagtgtaaacttaaccactgatttatcagttctacttaggtag

SEQ ID NO: 261 - DNA, 129 bp, synthetic construct
  source 1..129: mol_type = other DNA
tattaaatactcgtattgctgtttaagaagtatgttagaaataacatactgagtttaaataagtgtaaacttaaccactgatttatcagttctacttaggtagaataaagtcctatttatagggctttt

SEQ ID NO: 262 - DNA, 123 bp, synthetic construct
  source 1..123: mol_type = other DNA
tattaaatactcgtattgctgtttgagagccttgttgaaaaacaaggcgagttcaaataaggattattccgaaaccgctttatagtctgcattgtgcaggttctcattgatccggcgaaggcc

SEQ ID NO: 263 - DNA, 99 bp, synthetic construct
  source 1..99: mol_type = other DNA
tattaaatactcgtattgctatttaagtacctgtaaggaaatttacagatctacttaaacaaggctttatgccgaaatcaaggacacgcttgcgtgtcc

SEQ ID NO: 264 - DNA, 99 bp, synthetic construct
  source 1..99: mol_type = other DNA
tattaaatactcgtattgctattttagtacctgaaaggaaattttcagatctactaaaacaaggctttatgccgaaatcaaggacacgcttgcgtgtcc

SEQ ID NO: 265 - DNA, 98 bp, synthetic construct
  source 1..98: mol_type = other DNA
tattaaatactcgtattgctatttaagtacctagtaggaaatattagatctacttaaacaaggctttatgccgaaatcaaggacaccgacgggtgtcc

SEQ ID NO: 266 - DNA, 123 bp, synthetic construct
  source 1..123: mol_type = other DNA
tattaaatactcgtattgctgtttgagaaccttgtagaaaacaaggtgagtgcaaataaggattttccgaaatcgccgtaaggctctgcatggtgcagattcaaaaggcctgtttacaggtct

SEQ ID NO: 267 - DNA, 123 bp, synthetic construct
  source 1..123: mol_type = other DNA
tattaaatactcgtattgctgtttgagagccctgtagaaatacagggtgagttcaaataaggctttagccgaaatcgcgcctcgtgtccgcattgtgcgggtttaaaagagaatcgtaagatt

SEQ ID NO: 268 - DNA, 123 bp, synthetic construct
  source 1..123: mol_type = other DNA
tattaaatactcgtattgctgtttgagaaccttgtagaaatacaaggtgagttcaaataaggagcaatccgaaattacattgaccgcattgtgcggatatgaaagacgtagcctatggctaag

SEQ ID NO: 269 - DNA, 123 bp, synthetic construct
  source 1..123: mol_type = other DNA
tattaaatactcgtattgctgtttgagaaccttgtagaaaacaaggtgagttcaaataaggatttatccgaaattacatttgcccgcattgtgcggattgaaaaggttcgacttatgtcgaat

SEQ ID NO: 270 - DNA, 101 bp, synthetic construct
  source 1..101: mol_type = other DNA
tattaaatactcgtattgctgtttaagtacctagtaagaaattattagacctacttaaataaggctttatgccgaaatcaagaacacccgacggggtgttc

SEQ ID NO: 271 - DNA, 99 bp, synthetic construct
  source 1..99: mol_type = other DNA
tattaaatactcgtattgctgtttaagtacctggtaggaaattatcagatctacttaaacaaggctttatgccgaaatcaaggacatcgacggatgtcc

SEQ ID NO: 272 - DNA, 123 bp, synthetic construct
  source 1..123: mol_type = other DNA
tattaaatactcgtattgctgtttgagaaccctgtagaaatacagggtgagttcaaataaggcattagccgaaattgcacctgatgtccgcattgtgcgggattaaagaggatcgaaggattc

SEQ ID NO: 273 - DNA, 123 bp, synthetic construct
  source 1..123: mol_type = other DNA
tattaaatactcgtattgctgtttgagagccctgtagaaatacagggtgagttcaaataaggctttagccgatatcgcacctcgtgtccgcattgtgcgggattttaagagaatcgtttcgat

SEQ ID NO: 274 - DNA, 123 bp, synthetic construct
  source 1..123: mol_type = other DNA
tattaaatactcgtattgctgtttgagagacttgttgaaaaacaggtcgagtgcaaataaggtttatccgaactcgtcaatttgatccgcattgtgcggtttttaaaatccgatgtttcatcg

SEQ ID NO: 275 - DNA, 123 bp, synthetic construct
  source 1..123: mol_type = other DNA
tattaaatactcgtattgctgtttgagagacttgttgaaaaacaggtcgagtgcaaataaggtttatccgaactcgtcaatttgatccgcattgtgcggttattaaaatccgatgtttcatcg

SEQ ID NO: 276 - DNA, 83 bp, synthetic construct
  source 1..83: mol_type = other DNA
tattaaatactcgtattgctgtttaagtacctggagagaaatttacttcagacctacttaaataaggatttattccgaaagaa

SEQ ID NO: 277 - DNA, 123 bp, synthetic construct
  source 1..123: mol_type = other DNA
tattaaatactcgtattgctgtttgagagccttgttgaaaaacaaggcgagttcaaataaggatttttccgaaatcgctatttagtctgcagtgtgcagtttcgtgtccggctttgccgggca

SEQ ID NO: 278 - DNA, 123 bp, synthetic construct
  source 1..123: mol_type = other DNA
tattaaatactcgtattgctgtttgagagccttgttgaaaaacaaggcgagttcaaataaggattattccgaaatcgctatttagtctgcagtgtgcagtttcgtgtccggctttgccgggca

SEQ ID NO: 279 - DNA, 123 bp, synthetic construct
  source 1..123: mol_type = other DNA
tattaaatactcgtattgctgtttgagagctttgttgaaaaacaaagcgagtgcaaataaggtttatccgaaatcgcttttatagcaagcattgtgcttattaaatctggctttccagccaga

SEQ ID NO: 280 - DNA, 123 bp, synthetic construct
  source 1..123: mol_type = other DNA
tattaaatactcgtattgctgtttgagagctttgttgaaaaacaaagcgagtgcaaataaggtttatccgaaatcgcttttatagcaagcatcgtgcttattaaatctggctttttagccaga

SEQ ID NO: 281 - DNA, 123 bp, synthetic construct
  source 1..123: mol_type = other DNA
tattaaatactcgtattgctgtttgagagctttgttgaaaaacaaagcgagtgcaaataaggtttatccgaaatcgcttttatagcaagcatcgtgcttattaaatctggcttattagccaga

SEQ ID NO: 282 - DNA, 123 bp, synthetic construct
  source 1..123: mol_type = other DNA
tattaaatactcgtattgctgtttgagagccctgtagaaatacagggtgagttcaaataaggctttagccgaaatcgcacctcgtgtccgcattgtgcgggtttaaagagaatcgtttgattc

SEQ ID NO: 283 - DNA, 123 bp, synthetic construct
  source 1..123: mol_type = other DNA
tattaaatactcgtattgctgtttgagagccctgtagaaatacagggtgagttcaaataaggctttagccgaaatcgcacctgagtgtccgcattgtgcgggattaagagaatcgaaagattc

SEQ ID NO: 284 - DNA, 123 bp, synthetic construct
  source 1..123: mol_type = other DNA
tattaaatactcgtattgctgtttgagaaccttgtagaaatacaaggtgagttcaaataaggatttatccgacatcgccgtaaggcccacattgtgtggtttaaaagacttgagaaagtcttt

SEQ ID NO: 285 - DNA, 81 bp, synthetic construct
  source 1..81: mol_type = other DNA
tattaaatactcgtattgctatttaagtacccggtgagaaaatttcttcggacctatttaaacaaggctttatgccgaaag

SEQ ID NO: 286 - DNA, 93 bp, synthetic construct
  source 1..93: mol_type = other DNA
tattaaatactcgtattgctgtttagttaccataagggaaattttatgacctaactaaacaagggtttatcccggattcggctcttcggagcc

SEQ ID NO: 287 - DNA, 93 bp, synthetic construct
  source 1..93: mol_type = other DNA
tattaaatactcgtattgctgttttgttaccataagggaaattttatgacctaacaaaacaagggtttatcccggattcggctcttcggagcc

SEQ ID NO: 288 - DNA, 93 bp, synthetic construct
  source 1..93: mol_type = other DNA
tattaaatactcgtattgctgtttagttaccataaaggaaattttatgacctaactaaacaagggtttatcccggattcggctcttcggagcc

SEQ ID NO: 289 - DNA, 103 bp, synthetic construct
  source 1..103: mol_type = other DNA
tattaaatactcgtattgctgttttagtattgtgttagaaatagcacaatgagttaaaataaggtttaaaccttaaatgccaacttgatgttggtttcactta

SEQ ID NO: 290 - DNA, 136 bp, synthetic construct
  source 1..136: mol_type = other DNA
tattaaatactcgtattgctgtttaagtattgtgttagaaatagcacaatgagtttaaataaggtttaaaccttaaatgccaacttgatgttggtttcacttaggtgaatttaagttctagttatctagagctttt

SEQ ID NO: 291 - DNA, 99 bp, synthetic construct
  source 1..99: mol_type = other DNA
tattaaatactcgtattgctatttaagtacctggtaagaaattatcagatctacttaaacaaggctttatgccgaaatcaaggacatcgatggatgtcc

SEQ ID NO: 292 - DNA, 99 bp, synthetic construct
  source 1..99: mol_type = other DNA
tattaaatactcgtattgctattttagtacctggaaagaaattttcagatctactaaaacaaggctttatgccgaaatcaaggacatcgatggatgtcc

SEQ ID NO: 293 - DNA, 101 bp, synthetic construct
  source 1..101: mol_type = other DNA
tattaaatactcgtattgctatttaagtacctgtaaggaaatttacagatctacttaaataaggctttatgccgaaatcaaggagtgccgacgggcactcc

SEQ ID NO: 294 - DNA, 101 bp, synthetic construct
  source 1..101: mol_type = other DNA
tattaaatactcgtattgctatttaagtacctggtaggaaattatcagatctacttaaacaaggctttatgccgaaatgaaaaggacatcctcggatgtcc

SEQ ID NO: 295 - DNA, 103 bp, synthetic construct
  source 1..103: mol_type = other DNA
tattaaatactcgtattgctgttttagaattgtgttagaaatagcacaatgagttaaaataagggtaaccttaaatgccaattcatattggtttcacttaggt

SEQ ID NO: 296 - DNA, 134 bp, synthetic construct
  source 1..134: mol_type = other DNA
tattaaatactcgtattgctgtttaagaattgtgttagaaatagcacaatgagtttaaataagggtaaccttaaatgccaattcatattggtttcacttaggtgaataaagagttctagcttgctagggctttt

SEQ ID NO: 297 - DNA, 123 bp, synthetic construct
  source 1..123: mol_type = other DNA
tattaaatactcgtattgctgtttgagaaccttgtagaaatacaaggtgagtgcaaataaggatttatccgacatcgccgtttggtctgcatggtgcaggtaaaagacttggtttcaagtctt

SEQ ID NO: 298 - DNA, 103 bp, synthetic construct
  source 1..103: mol_type = other DNA
tattaaatactcgtattgctgtttaagttctatgtcagaaatgacatagagagtttaaataaaggttttaccctaaatgctcacgtatgtgttcggctgctta

SEQ ID NO: 299 - DNA, 101 bp, synthetic construct
  source 1..101: mol_type = other DNA
tattaaatactcgtattgctatttaagtacctgtaaggaaatttacagatctacttaaataaggctttatgccgagattaaggagtgccgacgggtactcc

SEQ ID NO: 300 - DNA, 97 bp, synthetic construct
  source 1..97: mol_type = other DNA
tattaaatactcgtattgctgtttagctaccatctatgaaagatatatgatctagctaaacaagggtttatcccggattcggctcctataaggagcc

SEQ ID NO: 301 - DNA, 103 bp, synthetic construct
  source 1..103: mol_type = other DNA
tattaaatactcgtattgctgttttagtattgtgttagaaatagcacaatgagttaaaataaggtttaaccttaaatgccaatttatgttggtttcacttagg

SEQ ID NO: 302 - DNA, 134 bp, synthetic construct
  source 1..134: mol_type = other DNA
tattaaatactcgtattgctgtttaagtattgtgttagaaatagcacaatgagtttaaataaggtttaaccttaaatgccaatttatgttggtttcacttaggtgaatttaagttctagttatctagagctttt

SEQ ID NO: 303 - DNA, 103 bp, synthetic construct
  source 1..103: mol_type = other DNA
tattaaatactcgtattgctgttttagaaatatgttagaaatagcatattgagttaaaataagtgtaaactataatgccaagtaattggtttcacttaggtga

SEQ ID NO: 304 - DNA, 128 bp, synthetic construct
  source 1..128: mol_type = other DNA
tattaaatactcgtattgctgtttaagaaatatgttagaaatagcatattgagtttaaataagtgtaaactataatgccaagtaattggtttcacttaggtgaagttagagcttttcgaagctctttt

SEQ ID NO: 305 - DNA, 99 bp, synthetic construct; identical to SEQ ID NO: 271
  source 1..99: mol_type = other DNA

SEQ ID NO: 306 - DNA, 123 bp, synthetic construct
  source 1..123: mol_type = other DNA
tattaaatactcgtattgctgtttgagagctttgttgaaaaacaaagcgagtgcaaataaggttaatccgaaatcgccactttgtggctctgcatggtgcagattttattggtctcatatgag

SEQ ID NO: 307 - DNA, 123 bp, synthetic construct
  source 1..123: mol_type = other DNA
tattaaatactcgtattgctgtttgagaaccttgtagaaatacaaggtgagttcaaataaggatttatccgaaattgcattttgcccacattgtgtggatttaaaaagaccgttagggtcttt

SEQ ID NO: 308 - DNA, 103 bp, synthetic construct
  source 1..103: mol_type = other DNA
tattaaatactcgtattgctgtttaagatctatgtcagaaatgacatagagagtttaaataaaggcttagccctaaaaatttacacttatgtgggtggttgtt

SEQ ID NO: 309 - DNA, 93 bp, synthetic construct
  source 1..93: mol_type = other DNA
tattaaatactcgtattgctgtttagttaccatatgagaaattatatgacctaactaaacaagggtttatcccggattcggctcttcggagcc

SEQ ID NO: 310 - DNA, 96 bp, synthetic construct
  source 1..96: mol_type = other DNA
tattaaatactcgtattgctgtttagctaccatatgggaaattatatgacctggctaaacaagggtttatcccggattcggctcctataaggagcc

SEQ ID NO: 311 - DNA, 103 bp, synthetic construct
  source 1..103: mol_type = other DNA
tattaaatactcgtattgctgttttagagttgtgttagaaatagcacaacgagttaaaataaggtttaaccttaaatgtcaattaaatattgattttcactta

SEQ ID NO: 312 - DNA, 136 bp, synthetic construct
  source 1..136: mol_type = other DNA
tattaaatactcgtattgctgtttaagagttgtgttagaaatagcacaacgagtttaaataaggtttaaccttaaatgtcaattaaatattgattttcacttaggtgattttaagttctagttttctagaactttt

SEQ ID NO: 313 - DNA, 103 bp, synthetic construct
  source 1..103: mol_type = other DNA
tattaaatactcgtattgctgtttaagttctatgtcagaaatgacatagagagtttaaataaaggcttagccctaaatgctcattaatgtgagaatgaaccgt

SEQ ID NO: 314 - DNA, 116 bp, synthetic construct
  source 1..116: mol_type = other DNA
tattaaatactcgtattgctgttttagttctatgtcagaaatgacatagagagttaaaataaaggcttagccctaaatgctcattaatgtgagaatgaaccgttagctggttcttt

SEQ ID NO: 315 - DNA, 93 bp, synthetic construct; identical to SEQ ID NO: 288
  source 1..93: mol_type = other DNA

SEQ ID NO: 316 - DNA, 99 bp, synthetic construct
  source 1..99: mol_type = other DNA
tattaaatactcgtattgctgttacaggaccctgtgagaaattacaggtttctgtaataaggcataatgccttaggtttgatctaaccttaaagatcat

SEQ ID NO: 317 - DNA, 101 bp, synthetic construct
  source 1..101: mol_type = other DNA
tattaaatactcgtattgctgttacagtaccctgtgagaaattacagtaccctgtagattttaagcaatcttaaacacggaacatttgatttatttctttg

SEQ ID NO: 318 - DNA, 99 bp, synthetic construct
  source 1..99: mol_type = other DNA
tattaaatactcgtattgctatttaagtacctgtagagaaatttacagatctacttaaacaaggctttatgccgaactcaaggacatcgaaagatgtcc

SEQ ID NO: 319 - DNA, 99 bp, synthetic construct
  source 1..99: mol_type = other DNA
tattaaatactcgtattgctattttagtacctgaagagaaattttcagatctactaaaacaaggctttatgccgaactcaaggacatcgaaagatgtcc

SEQ ID NO: 320 - DNA, 98 bp, synthetic construct
  source 1..98: mol_type = other DNA
tattaaatactcgtattgctgtttaagtacccttagagaaatttaaggacctacttaaataaggctttatgccggattcaaagacaccgacgggtgtc

SEQ ID NO: 321 - DNA, 123 bp, synthetic construct
  source 1..123: mol_type = other DNA
tattaaatactcgtattgctgtttgagaaccttgtagaaaacaagtgtgagtgcaaataaggattttccgaaatcgccgaaaggcctgcatggtgcagattaaaagacttgacttttcaagtc

SEQ ID NO: 322 - DNA, 99 bp, synthetic construct
  source 1..99: mol_type = other DNA
tattaaatactcgtattgctatttaagtacctgtaaagaaatttacagatctgcttaaacaaggctttatgccgaaatcaaggacatcgacggatgtcc

SEQ ID NO: 323 - DNA, 103 bp, synthetic construct
  source 1..103: mol_type = other DNA
tattaaatactcgtattgctgttttagatttatgttagaaatagcataaatgagttaaaataaggtaaaaccttaaatgccaatttaatattggttttgctta

SEQ ID NO: 324 - DNA, 135 bp, synthetic construct
  source 1..135: mol_type = other DNA
tattaaatactcgtattgctgtttaagatttatgttagaaatagcataaatgagtttaaataaggtaaaaccttaaatgccaatttaatattggttttgcttaggcaagtaaagtcttagttatctaaggctttt

SEQ ID NO: 325 - DNA, 123 bp, synthetic construct
  source 1..123: mol_type = other DNA
tattaaatactcgtattgctgtttgagaaccttgtagaaatgcaaggtgagttcaaataaggatttatccgaaatcgccgtacggcccacattgtgtggatgaaaagacttggtttccaggtc

SEQ ID NO: 326 - DNA, 103 bp, synthetic construct
  source 1..103: mol_type = other DNA
tattaaatactcgtattgctgtttaagatctatgtcagaaatgacatagagagtttaaataaagctttaagcctaaatgctcacttatgtgttctgaaccgtt

SEQ ID NO: 327 - RNA, 123 nt, synthetic construct
  source 1..123: mol_type = other RNA
tattaaatactcgtattgctgtttgagagacttgtagaaatacaagacgagtgcaaataaggatttatccgaaatcgtcgcaagactctacatggtgtagaacttaaaagacttggtttcaag

SEQ ID NO: 328 - RNA, 81 nt, synthetic construct
  source 1..81: mol_type = other RNA
tattaaatactcgtattgctatttaagtacctggagagaaattatcttcagacctacttaaacaaggctttatgccgaaag

SEQ ID NO: 329 - RNA, 103 nt, synthetic construct
  source 1..103: mol_type = other RNA
tattaaatactcgtattgctgttttagaattgtgttagaaatagcacaatgagttaaaataaggttaaaccttaaatgccagttttctggtttcacttaggtg

SEQ ID NO: 330 - RNA, 131 nt, synthetic construct
  source 1..131: mol_type = other RNA
tattaaatactcgtattgctgtttaagaattgtgttagaaatagcacaatgagtttaaataaggttaaaccttaaatgccagttttctggtttcacttaggtgaattaagttctgtgtaaacagaactttt

SEQ ID NO: 331 - RNA, 103 nt, synthetic construct
  source 1..103: mol_type = other RNA
tattaaatactcgtattgctgtttaagatctatgtcagaaatgacatagagagtttaaataaaggtttaaccctaaatactctatttgagcgactgctttggc

SEQ ID NO: 332 - RNA, 103 nt, synthetic construct
  source 1..103: mol_type = other RNA
tattaaatactcgtattgctgtttaagatctatgtcagaaatgacatagagagtttaaataaagctttaagcctaaatgctcacgtatgtgttcgaacagtta

SEQ ID NO: 333 - RNA, 99 nt, synthetic construct
  source 1..99: mol_type = other RNA
tattaaatactcgtattgctatttaagtacctgtaaggaaatttacagatctacttaaacaaggctttatgccgaaatcaaggacatcgacggatgtcc

SEQ ID NO: 334 - RNA, 100 nt, synthetic construct
  source 1..100: mol_type = other RNA
tattaaatactcgtattgctatttaagtacctgtaaagaaatttacagaatctacttaaacaaggctttatgccgaaatcaaggacatcgacggatgtcc

SEQ ID NO: 335 - RNA, 93 nt, synthetic construct
  source 1..93: mol_type = other RNA
tattaaatactcgtattgctgtttagttaccatatgggaaactatatgacctagctaaacaagggtttatcccggattcggctcttcggagcc

SEQ ID NO: 336 - RNA, 123 nt, synthetic construct
  source 1..123: mol_type = other RNA
tattaaatactcgtattgctgtttgagagtcttgttgaaaaacaagacgagtgcaaataaggtttaaccgtaatcgtcgaaagactctgcatggtgcagaaagaaagaggctgtataaagcct

SEQ ID NO: 337 - RNA, 103 nt, synthetic construct
  source 1..103: mol_type = other RNA
tattaaatactcgtattgctgtttaagatctatgtcagaaatgacatagagagtttaaataaagctttaagcctaaatgctcacatatgtgttcagacggtta

SEQ ID NO: 338 - RNA, 103 nt, synthetic construct
  source 1..103: mol_type = other RNA
tattaaatactcgtattgctgtttaagctctatgttagaaatgacatagataagtttaaataaaagtttcctatatggatactctaaacacttactattgtag

SEQ ID NO: 339 - RNA, 125 nt, synthetic construct
  source 1..125: mol_type = other RNA
tattaaatactcgtattgctgttttagctctatgttagaaatgacatagataagttaaaataaaagtttcctatatggatactctaaacacttactattgtaggttgctgcgtgtgcagctcttt

SEQ ID NO: 340 - RNA, 101 nt, synthetic construct
  source 1..101: mol_type = other RNA
tattaaatactcgtattgctgttacagtaccctgtgagaaattacaggttactgtaataaggcataatgccttaagtgtttgtcgaccactacttgtcgac

SEQ ID NO: 341 - RNA, 103 nt, synthetic construct
  source 1..103: mol_type = other RNA
tattaaatactcgtattgctgtttaagatctatgtcagaaatgacatagagagtttaaataaagctttaagccttaatactcacttagtgagggacggatagc

SEQ ID NO: 342 - RNA, 103 nt, synthetic construct
  source 1..103: mol_type = other RNA
tattaaatactcgtattgctgtttaagatctatgtcagaaatgacatagaaaagtttaaataaaggcttagccctaaatactcctttggagaacgactgtgta

SEQ ID NO: 343 - RNA, 103 nt, synthetic construct
  source 1..103: mol_type = other RNA
tattaaatactcgtattgctgtttaagatctatgtcagaaatgacatagagagtttaaataaaggttttaccctaaatactcttctgagttgctgcttaggca

SEQ ID NO: 344 - RNA, 93 nt, synthetic construct
  source 1..93: mol_type = other RNA
tattaaatactcgtattgctgtttagttaccatatgggaaactatatgacctaactaaacaagggtttatcccggattcggctcttcggagcc

SEQ ID NO: 345 - RNA, 93 nt, synthetic construct
  source 1..93: mol_type = other RNA
tattaaatactcgtattgctgttttgttaccatatgggaaactatatgacctaacaaaacaagggtttatcccggattcggctcttcggagcc

SEQ ID NO: 346 - RNA, 103 nt, synthetic construct
  source 1..103: mol_type = other RNA
tattaaatactcgtattgctgttttagaagtatgttagaaataacataccgagttaaaataaggtttaaccttaaatactggctatgccagattcatttagat

SEQ ID NO: 347 - RNA, 131 nt, synthetic construct
  source 1..131: mol_type = other RNA
tattaaatactcgtattgctgtttaagaagtatgttagaaataacataccgagtttaaataaggtttaaccttaaatactggctatgccagattcatttagatgaagtagcgcctctttggaggtgcttta

SEQ ID NO: 348 - RNA, 97 nt, synthetic construct
  source 1..97: mol_type = other RNA
tattaaatactcgtattgctgttacaggaccctgtatgaaaatacaggtttctgtaataaggcataatgccttaggcttaatcccccttaaggattt

SEQ ID NO: 349 - RNA, 103 nt, synthetic construct
  source 1..103: mol_type = other RNA
tattaaatactcgtattgctgtttaagttctatgtcagaaatgacatagagagtttaaataaaggattacctaattactcattatgtgatgagaggctgctta

SEQ ID NO: 350 - RNA, 115 nt, synthetic construct
  source 1..115: mol_type = other RNA
tattaaatactcgtattgctgttttagttctatgtcagaaatgacatagagagttaaaataaaggattacctaattactcattatgtgatgagaggctgcttaggctggcctttt

SEQ ID NO: 351 - RNA, 103 nt, synthetic construct
  source 1..103: mol_type = other RNA
tattaaatactcgtattgctgttttagaattatgttagaaatagcataatgagttaaaataaggttaaacctttaatgctgatttatatcagtttcgcttagg

SEQ ID NO: 352 - RNA, 129 nt, synthetic construct
  source 1..129: mol_type = other RNA
tattaaatactcgtattgctgtttaagaattatgttagaaatagcataatgagtttaaataaggttaaacctttaatgctgatttatatcagtttcgcttaggtgaaatgagctctttgagctcttttt

SEQ ID NO: 353 - RNA, 103 nt, synthetic construct
  source 1..103: mol_type = other RNA
tattaaatactcgtattgctgtttaagatctatgtcagaaatgacatagagagtttaaataaagctttaagcctaaatgctcacttgtgttcggacggttagc

SEQ ID NO: 354 - RNA, 123 nt, synthetic construct
  source 1..123: mol_type = other RNA
tattaaatactcgtattgctgtttgagagtattgtagaaatacaagacgagtgcaaataaggtttatccgaaatcgtcgtatgactctacatggtgtagataataaaaaggacttgcgtgcaa

SEQ ID NO: 355 - RNA, 123 nt, synthetic construct
  source 1..123: mol_type = other RNA
tattaaatactcgtattgctgtttgagagacttgtagaaatacaagacgagtgcaaataaggatttatccgaaatcgtcgcaagactctacatggtgtagaacttaaaaggcttggtttcaag

SEQ ID NO: 356 - RNA, 123 nt, synthetic construct
  source 1..123: mol_type = other RNA
tattaaatactcgtattgctgtttgagaaccttgtagaaatacaaggtgagttcaaataaggattttatccgacatcgccgtaaggttagcattgtgcgacagaaaagacttcttcggaagtc

SEQ ID NO: 357 - RNA, 93 nt, synthetic construct
  source 1..93: mol_type = other RNA
tattaaatactcgtattgctgtttaattaccattaatgaaattatatgacctaattaaacaagggtttatcccggattcggctcttcggagcc

SEQ ID NO: 358 - RNA, 93 nt, synthetic construct
  source 1..93: mol_type = other RNA
tattaaatactcgtattgctgttttattaccattaatgaaattatatgacctaataaaacaagggtttatcccggattcggctcttcggagcc

SEQ ID NO: 359 - RNA, 103 nt, synthetic construct
  source 1..103: mol_type = other RNA
tattaaatactcgtattgctgttttagaagtatgttagaaataacatactgagttaaaataagtgtaaacttaaccactgatttatcagttctacttaggtag

SEQ ID NO: 360 - RNA, 129 nt, synthetic construct
  source 1..129: mol_type = other RNA
tattaaatactcgtattgctgtttaagaagtatgttagaaataacatactgagtttaaataagtgtaaacttaaccactgatttatcagttctacttaggtagaataaagtcctatttatagggctttt

SEQ ID NO: 361 - RNA, 123 nt, synthetic construct
  source 1..123: mol_type = other RNA
tattaaatactcgtattgctgtttgagagccttgttgaaaaacaaggcgagttcaaataaggattattccgaaaccgctttatagtctgcattgtgcaggttctcattgatccggcgaaggcc

SEQ ID NO: 362 - RNA, 99 nt, synthetic construct
  source 1..99: mol_type = other RNA
tattaaatactcgtattgctatttaagtacctgtaaggaaatttacagatctacttaaacaaggctttatgccgaaatcaaggacacgcttgcgtgtcc

SEQ ID NO: 363 - RNA, 99 nt, synthetic construct
  source 1..99: mol_type = other RNA
tattaaatactcgtattgctattttagtacctgaaaggaaattttcagatctactaaaacaaggctttatgccgaaatcaaggacacgcttgcgtgtcc

SEQ ID NO: 364 - RNA, 98 nt, synthetic construct
  source 1..98: mol_type = other RNA
tattaaatactcgtattgctatttaagtacctagtaggaaatattagatctacttaaacaaggctttatgccgaaatcaaggacaccgacgggtgtcc

SEQ ID NO: 365 - RNA, 123 nt, synthetic construct
  source 1..123: mol_type = other RNA
tattaaatactcgtattgctgtttgagaaccttgtagaaaacaaggtgagtgcaaataaggattttccgaaatcgccgtaaggctctgcatggtgcagattcaaaaggcctgtttacaggtct

SEQ ID NO: 366 - RNA, 123 nt, synthetic construct
  source 1..123: mol_type = other RNA
tattaaatactcgtattgctgtttgagagccctgtagaaatacagggtgagttcaaataaggctttagccgaaatcgcgcctcgtgtccgcattgtgcgggtttaaaagagaatcgtaagatt

SEQ ID NO: 367 - RNA, 123 nt, synthetic construct
  source 1..123: mol_type = other RNA
tattaaatactcgtattgctgtttgagaaccttgtagaaatacaaggtgagttcaaataaggagcaatccgaaattacattgaccgcattgtgcggatatgaaagacgtagcctatggctaag

SEQ ID NO: 368 - RNA, 123 nt, synthetic construct
  source 1..123: mol_type = other RNA
tattaaatactcgtattgctgtttgagaaccttgtagaaaacaaggtgagttcaaataaggatttatccgaaattacatttgcccgcattgtgcggattgaaaaggttcgacttatgtcgaat

SEQ ID NO: 369 - RNA, 101 nt, synthetic construct
  source 1..101: mol_type = other RNA
tattaaatactcgtattgctgtttaagtacctagtaagaaattattagacctacttaaataaggctttatgccgaaatcaagaacacccgacggggtgttc

SEQ ID NO: 370 - RNA, 99 nt, synthetic construct
  source 1..99: mol_type = other RNA
tattaaatactcgtattgctgtttaagtacctggtaggaaattatcagatctacttaaacaaggctttatgccgaaatcaaggacatcgacggatgtcc

SEQ ID NO: 371 - RNA, 123 nt, synthetic construct
  source 1..123: mol_type = other RNA
tattaaatactcgtattgctgtttgagaaccctgtagaaatacagggtgagttcaaataaggcattagccgaaattgcacctgatgtccgcattgtgcgggattaaagaggatcgaaggattc

SEQ ID NO: 372 - RNA, 123 nt, synthetic construct
  source 1..123: mol_type = other RNA
tattaaatactcgtattgctgtttgagagccctgtagaaatacagggtgagttcaaataaggctttagccgatatcgcacctcgtgtccgcattgtgcgggattttaagagaatcgtttcgat

SEQ ID NO: 373 - RNA, 123 nt, synthetic construct
  source 1..123: mol_type = other RNA
tattaaatactcgtattgctgtttgagagacttgttgaaaaacaggtcgagtgcaaataaggtttatccgaactcgtcaatttgatccgcattgtgcggtttttaaaatccgatgtttcatcg

SEQ ID NO: 374 - RNA, 123 nt, synthetic construct
  source 1..123: mol_type = other RNA
tattaaatactcgtattgctgtttgagagacttgttgaaaaacaggtcgagtgcaaataaggtttatccgaactcgtcaatttgatccgcattgtgcggttattaaaatccgatgtttcatcg

SEQ ID NO: 375 - RNA, 83 nt, synthetic construct
  source 1..83: mol_type = other RNA
tattaaatactcgtattgctgtttaagtacctggagagaaatttacttcagacctacttaaataaggatttattccgaaagaa

SEQ ID NO: 376 - RNA, 123 nt, synthetic construct
  source 1..123: mol_type = other RNA
tattaaatactcgtattgctgtttgagagccttgttgaaaaacaaggcgagttcaaataaggatttttccgaaatcgctatttagtctgcagtgtgcagtttcgtgtccggctttgccgggca

SEQ ID NO: 377 - RNA, 123 nt, synthetic construct
  source 1..123: mol_type = other RNA
tattaaatactcgtattgctgtttgagagccttgttgaaaaacaaggcgagttcaaataaggattattccgaaatcgctatttagtctgcagtgtgcagtttcgtgtccggctttgccgggca

SEQ ID NO: 378 - RNA, 123 nt, synthetic construct
  source 1..123: mol_type = other RNA
tattaaatactcgtattgctgtttgagagctttgttgaaaaacaaagcgagtgcaaataaggtttatccgaaatcgcttttatagcaagcattgtgcttattaaatctggctttccagccaga

SEQ ID NO: 379 - RNA, 123 nt, synthetic construct
  source 1..123: mol_type = other RNA
tattaaatactcgtattgctgtttgagagctttgttgaaaaacaaagcgagtgcaaataaggtttatccgaaatcgcttttatagcaagcatcgtgcttattaaatctggctttttagccaga

SEQ ID NO: 380 - RNA, 123 nt, synthetic construct
  source 1..123: mol_type = other RNA
tattaaatactcgtattgctgtttgagagctttgttgaaaaacaaagcgagtgcaaataaggtttatccgaaatcgcttttatagcaagcatcgtgcttattaaatctggcttattagccaga

SEQ ID NO: 381 - RNA, 123 nt, synthetic construct
  source 1..123: mol_type = other RNA
tattaaatactcgtattgctgtttgagagccctgtagaaatacagggtgagttcaaataaggctttagccgaaatcgcacctcgtgtccgcattgtgcgggtttaaagagaatcgtttgattc

SEQ ID NO: 382 - RNA, 123 nt, synthetic construct
  source 1..123: mol_type = other RNA
tattaaatactcgtattgctgtttgagagccctgtagaaatacagggtgagttcaaataaggctttagccgaaatcgcacctgagtgtccgcattgtgcgggattaagagaatcgaaagattc

SEQ ID NO: 383 - RNA, 123 nt, synthetic construct
  source 1..123: mol_type = other RNA
tattaaatactcgtattgctgtttgagaaccttgtagaaatacaaggtgagttcaaataaggatttatccgacatcgccgtaaggcccacattgtgtggtttaaaagacttgagaaagtcttt

SEQ ID NO: 384 - RNA, 81 nt, synthetic construct
  source 1..81: mol_type = other RNA
tattaaatactcgtattgctatttaagtacccggtgagaaaatttcttcggacctatttaaacaaggctttatgccgaaag

SEQ ID NO: 385 - RNA, 93 nt, synthetic construct
  source 1..93: mol_type = other RNA
tattaaatactcgtattgctgtttagttaccataagggaaattttatgacctaactaaacaagggtttatcccggattcggctcttcggagcc

SEQ ID NO: 386 - RNA, 93 nt, synthetic construct
  source 1..93: mol_type = other RNA
tattaaatactcgtattgctgttttgttaccataagggaaattttatgacctaacaaaacaagggtttatcccggattcggctcttcggagcc

SEQ ID NO: 387 - RNA, 93 nt, synthetic construct
  source 1..93: mol_type = other RNA
tattaaatactcgtattgctgtttagttaccataaaggaaattttatgacctaactaaacaagggtttatcccggattcggctcttcggagcc

SEQ ID NO: 388 - RNA, 103 nt, synthetic construct
  source 1..103: mol_type = other RNA
tattaaatactcgtattgctgttttagtattgtgttagaaatagcacaatgagttaaaataaggtttaaaccttaaatgccaacttgatgttggtttcactta

SEQ ID NO: 389 - RNA, 136 nt, synthetic construct
  source 1..136: mol_type = other RNA
tattaaatactcgtattgctgtttaagtattgtgttagaaatagcacaatgagtttaaataaggtttaaaccttaaatgccaacttgatgttggtttcacttaggtgaatttaagttctagttatctagagctttt

SEQ ID NO: 390 - RNA, 99 nt, synthetic construct
  source 1..99: mol_type = other RNA
tattaaatactcgtattgctatttaagtacctggtaagaaattatcagatctacttaaacaaggctttatgccgaaatcaaggacatcgatggatgtcc

SEQ ID NO: 391 - RNA, 99 nt, synthetic construct
  source 1..99: mol_type = other RNA
tattaaatactcgtattgctattttagtacctggaaagaaattttcagatctactaaaacaaggctttatgccgaaatcaaggacatcgatggatgtcc

SEQ ID NO: 392 - RNA, 101 nt, synthetic construct
  source 1..101: mol_type = other RNA
tattaaatactcgtattgctatttaagtacctgtaaggaaatttacagatctacttaaataaggctttatgccgaaatcaaggagtgccgacgggcactcc

SEQ ID NO: 393 - RNA, 101 nt, synthetic construct
  source 1..101: mol_type = other RNA
tattaaatactcgtattgctatttaagtacctggtaggaaattatcagatctacttaaacaaggctttatgccgaaatgaaaaggacatcctcggatgtcc

SEQ ID NO: 394 - RNA, 103 nt, synthetic construct
  source 1..103: mol_type = other RNA
tattaaatactcgtattgctgttttagaattgtgttagaaatagcacaatgagttaaaataagggtaaccttaaatgccaattcatattggtttcacttaggt

SEQ ID NO: 395 - RNA, 134 nt, synthetic construct
  source 1..134: mol_type = other RNA
tattaaatactcgtattgctgtttaagaattgtgttagaaatagcacaatgagtttaaataagggtaaccttaaatgccaattcatattggtttcacttaggtgaataaagagttctagcttgctagggctttt

SEQ ID NO: 396 - RNA, 123 nt, synthetic construct
  source 1..123: mol_type = other RNA
tattaaatactcgtattgctgtttgagaaccttgtagaaatacaaggtgagtgcaaataaggatttatccgacatcgccgtttggtctgcatggtgcaggtaaaagacttggtttcaagtctt

SEQ ID NO: 397 - RNA, 103 nt, synthetic construct
  source 1..103: mol_type = other RNA
tattaaatactcgtattgctgtttaagttctatgtcagaaatgacatagagagtttaaataaaggttttaccctaaatgctcacgtatgtgttcggctgctta

SEQ ID NO: 398 - RNA, 101 nt, synthetic construct
  source 1..101: mol_type = other RNA
tattaaatactcgtattgctatttaagtacctgtaaggaaatttacagatctacttaaataaggctttatgccgagattaaggagtgccgacgggtactcc

SEQ ID NO: 399 - RNA, 97 nt, synthetic construct
  source 1..97: mol_type = other RNA
tattaaatactcgtattgctgtttagctaccatctatgaaagatatatgatctagctaaacaagggtttatcccggattcggctcctataaggagcc

SEQ ID NO: 400 - RNA, 103 nt, synthetic construct
  source 1..103: mol_type = other RNA
tattaaatactcgtattgctgttttagtattgtgttagaaatagcacaatgagttaaaataaggtttaaccttaaatgccaatttatgttggtttcacttagg

SEQ ID NO: 401 - RNA, 134 nt, synthetic construct
  source 1..134: mol_type = other RNA
tattaaatactcgtattgctgtttaagtattgtgttagaaatagcacaatgagtttaaataaggtttaaccttaaatgccaatttatgttggtttcacttaggtgaatttaagttctagttatctagagctttt

SEQ ID NO: 402 - RNA, 103 nt, synthetic construct
  source 1..103: mol_type = other RNA
tattaaatactcgtattgctgttttagaaatatgttagaaatagcatattgagttaaaataagtgtaaactataatgccaagtaattggtttcacttaggtga

SEQ ID NO: 403 - RNA, 128 nt, synthetic construct
  source 1..128: mol_type = other RNA
tattaaatactcgtattgctgtttaagaaatatgttagaaatagcatattgagtttaaataagtgtaaactataatgccaagtaattggtttcacttaggtgaagttagagcttttcgaagctctttt

SEQ ID NO: 404 - RNA, 99 nt, synthetic construct; identical to SEQ ID NO: 370
  source 1..99: mol_type = other RNA

SEQ ID NO: 405 - RNA, 123 nt, synthetic construct
  source 1..123: mol_type = other RNA
tattaaatactcgtattgctgtttgagagctttgttgaaaaacaaagcgagtgcaaataaggttaatccgaaatcgccactttgtggctctgcatggtgcagattttattggtctcatatgag

SEQ ID NO: 406 - RNA, 123 nt, synthetic construct
  source 1..123: mol_type = other RNA
tattaaatactcgtattgctgtttgagaaccttgtagaaatacaaggtgagttcaaataaggatttatccgaaattgcattttgcccacattgtgtggatttaaaaagaccgttagggtcttt

SEQ ID NO: 407 - RNA, 103 nt, synthetic construct
  source 1..103: mol_type = other RNA
tattaaatactcgtattgctgtttaagatctatgtcagaaatgacatagagagtttaaataaaggcttagccctaaaaatttacacttatgtgggtggttgtt

SEQ ID NO: 408 - RNA, 93 nt, synthetic construct
  source 1..93: mol_type = other RNA
tattaaatactcgtattgctgtttagttaccatatgagaaattatatgacctaactaaacaagggtttatcccggattcggctcttcggagcc

SEQ ID NO: 409 - RNA, 96 nt, synthetic construct
  source 1..96: mol_type = other RNA
tattaaatactcgtattgctgtttagctaccatatgggaaattatatgacctggctaaacaagggtttatcccggattcggctcctataaggagcc

SEQ ID NO: 410 - RNA, 103 nt, synthetic construct
  source 1..103: mol_type = other RNA
tattaaatactcgtattgctgttttagagttgtgttagaaatagcacaacgagttaaaataaggtttaaccttaaatgtcaattaaatattgattttcactta

SEQ ID NO: 411 - RNA, 136 nt, synthetic construct
  source 1..136: mol_type = other RNA
tattaaatactcgtattgctgtttaagagttgtgttagaaatagcacaacgagtttaaataaggtttaaccttaaatgtcaattaaatattgattttcacttaggtgattttaagttctagttttctagaactttt

SEQ ID NO: 412 - RNA, 103 nt, synthetic construct
  source 1..103: mol_type = other RNA
tattaaatactcgtattgctgtttaagttctatgtcagaaatgacatagagagtttaaataaaggcttagccctaaatgctcattaatgtgagaatgaaccgt

SEQ ID NO: 413 - RNA, 116 nt, synthetic construct
  source 1..116: mol_type = other RNA
tattaaatactcgtattgctgttttagttctatgtcagaaatgacatagagagttaaaataaaggcttagccctaaatgctcattaatgtgagaatgaaccgttagctggttcttt

SEQ ID NO: 414 - RNA, 93 nt, synthetic construct; identical to SEQ ID NO: 387
  source 1..93: mol_type = other RNA

SEQ ID NO: 415 - RNA, 99 nt, synthetic construct
  source 1..99: mol_type = other RNA
tattaaatactcgtattgctgttacaggaccctgtgagaaattacaggtttctgtaataaggcataatgccttaggtttgatctaaccttaaagatcat

SEQ ID NO: 416 - RNA, 101 nt, synthetic construct
  source 1..101: mol_type = other RNA
tattaaatactcgtattgctgttacagtaccctgtgagaaattacagtaccctgtagattttaagcaatcttaaacacggaacatttgatttatttctttg

SEQ ID NO: 417 - RNA, 99 nt, synthetic construct
  source 1..99: mol_type = other RNA
tattaaatactcgtattgctatttaagtacctgtagagaaatttacagatctacttaaacaaggctttatgccgaactcaaggacatcgaaagatgtcc

SEQ ID NO: 418 - RNA, 99 nt, synthetic construct
  source 1..99: mol_type = other RNA
tattaaatactcgtattgctattttagtacctgaagagaaattttcagatctactaaaacaaggctttatgccgaactcaaggacatcgaaagatgtcc

SEQ ID NO: 419 - RNA, 98 nt, synthetic construct
  source 1..98: mol_type = other RNA
tattaaatactcgtattgctgtttaagtacccttagagaaatttaaggacctacttaaataaggctttatgccggattcaaagacaccgacgggtgtc

SEQ ID NO: 420 - RNA, 123 nt, synthetic construct
  source 1..123: mol_type = other RNA
tattaaatactcgtattgctgtttgagaaccttgtagaaaacaagtgtgagtgcaaataaggattttccgaaatcgccgaaaggcctgcatggtgcagattaaaagacttgacttttcaagtc

SEQ ID NO: 421 - RNA, 99 nt, synthetic construct
  source 1..99: mol_type = other RNA
tattaaatactcgtattgctatttaagtacctgtaaagaaatttacagatctgcttaaacaaggctttatgccgaaatcaaggacatcgacggatgtcc

SEQ ID NO: 422 - RNA, 103 nt, synthetic construct
  source 1..103: mol_type = other RNA
tattaaatactcgtattgctgttttagatttatgttagaaatagcataaatgagttaaaataaggtaaaaccttaaatgccaatttaatattggttttgctta

SEQ ID NO: 423 - RNA, 135 nt, synthetic construct
  source 1..135: mol_type = other RNA
tattaaatactcgtattgctgtttaagatttatgttagaaatagcataaatgagtttaaataaggtaaaaccttaaatgccaatttaatattggttttgcttaggcaagtaaagtcttagttatctaaggctttt

SEQ ID NO: 424 - RNA, 123 nt, synthetic construct
  source 1..123: mol_type = other RNA
tattaaatactcgtattgctgtttgagaaccttgtagaaatgcaaggtgagttcaaataaggatttatccgaaatcgccgtacggcccacattgtgtggatgaaaagacttggtttccaggtc

SEQ ID NO: 425 - RNA, 103 nt, synthetic construct
  source 1..103: mol_type = other RNA
tattaaatactcgtattgctgtttaagatctatgtcagaaatgacatagagagtttaaataaagctttaagcctaaatgctcacttatgtgttctgaaccgtt

SEQ ID NO: 426 - protein, 166 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 427 - protein, 32 aa, synthetic construct
SGGSSGGSSGSETPGTSESATPESSGGSSGGS

SEQ ID NO: 428 - protein, 20 aa, synthetic construct
  VARIANT 4: note = K, H, or R
  VARIANT 8: note = K, H, or R
  VARIANT 11: note = K, H, or R
  VARIANT 15: note = K, H, or R
  VARIANT 19: note = K, H, or R
GLFXALLXLLXSLWXLLLXA

SEQ ID NO: 429 - protein, 20 aa, synthetic construct
GLFHALLHLLHSLWHLLLHA

SEQ ID NO: 430 - DNA, 19 bp, synthetic construct
  source 1..19: mol_type = other DNA
acatgaggatcacccatgt

SEQ ID NO: 431 - DNA, 25 bp, synthetic construct
  source 1..25: mol_type = other DNA
ggagcagacgatatggcgtcgctcc

SEQ ID NO: 432 - DNA, 15 bp, synthetic construct
  source 1..15: mol_type = other DNA
gccctgaagaagggc

SEQ ID NO: 433 - protein, 16 aa, synthetic construct
KRPAATKKAGQAKKKK

SEQ ID NO: 434 - protein, 7 aa, synthetic construct
PKKKRKV

SEQ ID NO: 435 - protein, 9 aa, synthetic construct
LPPLERLTL

SEQ ID NO: 436 - RNA, 123 nt, synthetic construct
  source 1..123: mol_type = other RNA
tggatgaaacccagacacatgtttgagagtcttgttgaaaaacaagacgagtgcaaataaggtttaaccgtaatcgtcgaaagactctgcatggtgcagaaagaaagaggctgtataaagcct

SEQ ID NO: 437 - RNA, 123 nt, synthetic construct
  source 1..123: mol_type = other RNA
agatacatctcataaatagtgtttgagagccctgtagaaatacagggtgagttcaaataaggctttagccgaaatcgcacctgagtgtccgcattgtgcgggattaagagaatcgaaagattc

SEQ ID NO: 438 - RNA, 123 nt, synthetic construct
  source 1..123: mol_type = other RNA
attcctcctcagatgccaccgtttgagagccctgtagaaatacagggtgagttcaaataaggctttagccgaaatcgcacctgagtgtccgcattgtgcgggattaagagaatcgaaagattc

SEQ ID NO: 439 - RNA, 123 nt, synthetic construct
  source 1..123: mol_type = other RNA
gggatcaagccgctgccaccgtttgagagccctgtagaaatacagggtgagttcaaataaggctttagccgaaatcgcacctgagtgtccgcattgtgcgggattaagagaatcgaaagattc

SEQ ID NO: 440 - RNA, 123 nt, synthetic construct
  source 1..123: mol_type = other RNA
tggatgaaacccagacacatgtttgagagccctgtagaaatacagggtgagttcaaataaggctttagccgaaatcgcacctgagtgtccgcattgtgcgggattaagagaatcgaaagattc

SEQ ID NO: 441 - RNA, 123 nt, synthetic construct
  source 1..123: mol_type = other RNA
cccagacacatagcaattcagtttgagagccctgtagaaatacagggtgagttcaaataaggctttagccgaaatcgcacctgagtgtccgcattgtgcgggattaagagaatcgaaagattc

SEQ ID NO: 442 - RNA, 123 nt, synthetic construct
  source 1..123: mol_type = other RNA
agaatggaaagtcaaatttcgtttgagagccctgtagaaatacagggtgagttcaaataaggctttagccgaaatcgcacctgagtgtccgcattgtgcgggattaagagaatcgaaagattc

SEQ ID NO: 443 - RNA, 123 nt, synthetic construct
  source 1..123: mol_type = other RNA
atccagcagagaatggaaaggtttgagagccctgtagaaatacagggtgagttcaaataaggctttagccgaaatcgcacctgagtgtccgcattgtgcgggattaagagaatcgaaagattc

SEQ ID NO: 444 - RNA, 123 nt, synthetic construct
  source 1..123: mol_type = other RNA
ccaccaaaaggcaaaggaatgtttgagagccctgtagaaatacagggtgagttcaaataaggctttagccgaaatcgcacctgagtgtccgcattgtgcgggattaagagaatcgaaagattc

SEQ ID NO: 445 - RNA, 123 nt, synthetic construct
  source 1..123: mol_type = other RNA
accttctcctttgggtccaggtttgagaaccctgtagaaatacagggtgagttcaaataaggcattagccgaaattgcacctgatgtccgcattgtgcgggattaaagaggatcgaaggattc

SEQ ID NO: 446 - RNA, 123 nt, synthetic construct
  source 1..123: mol_type = other RNA
gggaaagccaggggctccaggtttgagaaccctgtagaaatacagggtgagttcaaataaggcattagccgaaattgcacctgatgtccgcattgtgcgggattaaagaggatcgaaggattc

SEQ ID NO: 447 - RNA, 123 nt, synthetic construct
  source 1..123: mol_type = other RNA
ggcagaagccatctgcaagggtttgagaaccctgtagaaatacagggtgagttcaaataaggcattagccgaaattgcacctgatgtccgcattgtgcgggattaaagaggatcgaaggattc

SEQ ID NO: 448 - RNA, 123 nt, synthetic construct
  source 1..123: mol_type = other RNA
catggtaaacggggccattagtttgagaaccctgtagaaatacagggtgagttcaaataaggcattagccgaaattgcacctgatgtccgcattgtgcgggattaaagaggatcgaaggattc

SEQ ID NO: 449 - RNA, 123 nt, synthetic construct
  source 1..123: mol_type = other RNA
tgtatatcccaggtttccccgtttgagaaccctgtagaaatacagggtgagttcaaataaggcattagccgaaattgcacctgatgtccgcattgtgcgggattaaagaggatcgaaggattc

SEQ ID NO: 450 - RNA, 99 nt, synthetic construct
  source 1..99: mol_type = other RNA
ccctggccccaccaagtggggtttaagtacctggtaggaaattatcagatctacttaaacaaggctttatgccgaaatcaaggacatcgacggatgtcc

SEQ ID NO: 451 - RNA, 99 nt, synthetic construct
  source 1..99: mol_type = other RNA
ggccccaccaagtgggacaagtttaagtacctggtaggaaattatcagatctacttaaacaaggctttatgccgaaatcaaggacatcgacggatgtcc

SEQ ID NO: 452 - RNA, 99 nt, synthetic construct
  source 1..99: mol_type = other RNA
tgagggcaaagccttgctgagtttaagtacctggtaggaaattatcagatctacttaaacaaggctttatgccgaaatcaaggacatcgacggatgtcc

SEQ ID NO: 453 - RNA, 101 nt, synthetic construct
  source 1..101: mol_type = other RNA
ggccccaccaagtgggacaaatttaagtacctgtaaggaaatttacagatctacttaaataaggctttatgccgagattaaggagtgccgacgggtactcc

SEQ ID NO: 454 - RNA, 101 nt, synthetic construct
  source 1..101: mol_type = other RNA
gatagaaattggatgtgaggatttaagtacctgtaaggaaatttacagatctacttaaataaggctttatgccgagattaaggagtgccgacgggtactcc

SEQ ID NO: 455 - RNA, 101 nt, synthetic construct
  source 1..101: mol_type = other RNA
tttacaacatagccacgaggatttaagtacctgtaaggaaatttacagatctacttaaataaggctttatgccgagattaaggagtgccgacgggtactcc

SEQ ID NO: 456 - RNA, 101 nt, synthetic construct
  source 1..101: mol_type = other RNA
tgagggcaaagccttgctgaatttaagtacctgtaaggaaatttacagatctacttaaataaggctttatgccgagattaaggagtgccgacgggtactcc

SEQ ID NO: 457 - RNA, 101 nt, synthetic construct
  source 1..101: mol_type = other RNA
tccttcacaggcagatattaatttaagtacctgtaaggaaatttacagatctacttaaataaggctttatgccgagattaaggagtgccgacgggtactcc

SEQ ID NO: 458 - RNA, 99 nt, synthetic construct
  source 1..99: mol_type = other RNA
caagtggggaatggcaagcaatttaagtacctagtaggaaattattagatctacttaaacaaggctttatgccgaaatcaaggacaccgacgggtgtcc

SEQ ID NO: 459 - RNA, 99 nt, synthetic construct
  source 1..99: mol_type = other RNA
tggggaagaaggtgtcttctatttaagtacctagtaggaaattattagatctacttaaacaaggctttatgccgaaatcaaggacaccgacgggtgtcc

SEQ ID NO: 460 - RNA, 99 nt, synthetic construct
  source 1..99: mol_type = other RNA
tgatagcattagctggccgcatttaagtacctagtaggaaattattagatctacttaaacaaggctttatgccgaaatcaaggacaccgacgggtgtcc

SEQ ID NO: 461 - RNA, 99 nt, synthetic construct
  source 1..99: mol_type = other RNA
agccacagctctgtctctgaatttaagtacctagtaggaaattattagatctacttaaacaaggctttatgccgaaatcaaggacaccgacgggtgtcc

SEQ ID NO: 462 - RNA, 99 nt, synthetic construct
  source 1..99: mol_type = other RNA
taacctaaaaggagcctaccatttaagtacctagtaggaaattattagatctacttaaacaaggctttatgccgaaatcaaggacaccgacgggtgtcc

SEQ ID NO: 463 - RNA, 81 nt, synthetic construct
  source 1..81: mol_type = other RNA
cgggaaccagatctctcaccatttaagtacccggtgagaaattatcttcggacctatttaaacaaggctttatgccgaaag

SEQ ID NO: 464 - RNA, 81 nt, synthetic construct
  source 1..81: mol_type = other RNA
cgattcaagtggggaatggcatttaagtacccggtgagaaattatcttcggacctatttaaacaaggctttatgccgaaag

SEQ ID NO: 465 - RNA, 81 nt, synthetic construct
  source 1..81: mol_type = other RNA
ttttggaaaccagcagaaagatttaagtacccggtgagaaattatcttcggacctatttaaacaaggctttatgccgaaag

SEQ ID NO: 466 - RNA, 93 nt, synthetic construct
  source 1..93: mol_type = other RNA
ggcaccaatgtagataccaagtttagttaccataaaggaaattttatgacctaactaaacaagggtttatcccggattcggctcttcggagcc

SEQ ID NO: 467 - RNA, 103 nt, synthetic construct
  source 1..103: mol_type = other RNA
agggaaggaccaggggatgggtttaagatctatgtcagaaatgacatagagagtttaaataaaggttttaccctaaatactcttctgagttgctgcttaggca

SEQ ID NO: 468 - RNA, 103 nt, synthetic construct
  source 1..103: mol_type = other RNA
gaggcggtgagccgggcagggtttaagatctatgtcagaaatgacatagagagtttaaataaaggttttaccctaaatactcttctgagttgctgcttaggca

SEQ ID NO: 469 - RNA, 103 nt, synthetic construct
  source 1..103: mol_type = other RNA
ggagaagaggggcaatgcaggtttaagatctatgtcagaaatgacatagagagtttaaataaaggttttaccctaaatactcttctgagttgctgcttaggca

SEQ ID NO: 470 - RNA, 103 nt, synthetic construct
  source 1..103: mol_type = other RNA
gtcttgctgctcctcccggcgtttaagatctatgtcagaaatgacatagagagtttaaataaaggttttaccctaaatactcttctgagttgctgcttaggca

SEQ ID NO: 471 - RNA, 103 nt, synthetic construct
  source 1..103: mol_type = other RNA
ccacctggcttcccaatgccgtttaagatctatgtcagaaatgacatagagagtttaaataaaggttttaccctaaatactcttctgagttgctgcttaggca

SEQ ID NO: 472 - RNA, 103 nt, synthetic construct
  source 1..103: mol_type = other RNA
cctggagggcctgggggtccgtttaagatctatgtcagaaatgacatagagagtttaaataaaggttttaccctaaatactcttctgagttgctgcttaggca

SEQ ID NO: 473 - RNA, 103 nt, synthetic construct
  source 1..103: mol_type = other RNA
gcccaactaatataaacaccgtttaagatctatgtcagaaatgacatagagagtttaaataaaggttttaccctaaatactcttctgagttgctgcttaggca

SEQ ID NO: 474 - RNA, 103 nt, synthetic construct
  source 1..103: mol_type = other RNA
accggcgccaagaatgggcagtttaagatctatgtcagaaatgacatagagagtttaaataaaggttttaccctaaatactcttctgagttgctgcttaggca

SEQ ID NO: 475 - RNA, 103 nt, synthetic construct
  source 1..103: mol_type = other RNA
agctgttttgaagactctccgtttaagatctatgtcagaaatgacatagagagtttaaataaaggttttaccctaaatactcttctgagttgctgcttaggca

SEQ ID NO: 476 - RNA, 103 nt, synthetic construct
  source 1..103: mol_type = other RNA
gaggtgcagtaattcaggccgtttaagatctatgtcagaaatgacatagagagtttaaataaaggttttaccctaaatactcttctgagttgctgcttaggca

SEQ ID NO: 477 - RNA, 103 nt, synthetic construct
  source 1..103: mol_type = other RNA
cagtttggcaggcgaccagtgttttagaattatgttagaaatagcataatgagttaaaataaggttaaacctttaatgctgatttatatcagtttcgcttagg

SEQ ID NO: 478 - RNA, 123 nt, synthetic construct
  source 1..123: mol_type = other RNA
agatacatctcataaatagtgtttgagaaccttgtagaaatacaaggtgagtgcaaataaggatttatccgacatcgccgtttggtctgcatggtgcaggtaaaagacttggtttcaagtctt

SEQ ID NO: 479 - RNA, 123 nt, synthetic construct
  source 1..123: mol_type = other RNA
attcctcctcagatgccaccgtttgagaaccttgtagaaatacaaggtgagtgcaaataaggatttatccgacatcgccgtttggtctgcatggtgcaggtaaaagacttggtttcaagtctt

SEQ ID NO: 480 - RNA, 123 nt, synthetic construct
  source 1..123: mol_type = other RNA
gggatcaagccgctgccaccgtttgagaaccttgtagaaatacaaggtgagtgcaaataaggatttatccgacatcgccgtttggtctgcatggtgcaggtaaaagacttggtttcaagtctt

SEQ ID NO: 481 - RNA, 123 nt, synthetic construct
  source 1..123: mol_type = other RNA
tggatgaaacccagacacatgtttgagaaccttgtagaaatacaaggtgagtgcaaataaggatttatccgacatcgccgtttggtctgcatggtgcaggtaaaagacttggtttcaagtctt

SEQ ID NO: 482 - RNA, 123 nt, synthetic construct
  source 1..123: mol_type = other RNA
cccagacacatagcaattcagtttgagaaccttgtagaaatacaaggtgagtgcaaataaggatttatccgacatcgccgtttggtctgcatggtgcaggtaaaagacttggtttcaagtctt

SEQ ID NO: 483 - RNA, 123 nt, synthetic construct
  source 1..123: mol_type = other RNA
gacttactgaagaatggagagtttgagaaccttgtagaaatacaaggtgagtgcaaataaggatttatccgacatcgccgtttggtctgcatggtgcaggtaaaagacttggtttcaagtctt

SEQ ID NO: 484 - RNA, 123 nt, synthetic construct
  source 1..123: mol_type = other RNA
atccagcagagaatggaaaggtttgagaaccttgtagaaatacaaggtgagtgcaaataaggatttatccgacatcgccgtttggtctgcatggtgcaggtaaaagacttggtttcaagtctt

SEQ ID NO: 485 - RNA, 99 nt, synthetic construct
  source 1..99: mol_type = other RNA
ccccaaattctaagcagagtatttaagtacctgtaaggaaatttacagatctacttaaacaaggctttatgccgaaatcaaggacatcgacggatgtcc

SEQ ID NO: 486 - RNA, 99 nt, synthetic construct
  source 1..99: mol_type = other RNA
ccctacattttgtgcataaaatttaagtacctgtaaggaaatttacagatctacttaaacaaggctttatgccgaaatcaaggacatcgacggatgtcc

SEQ ID NO: 487 - RNA, 99 nt, synthetic construct
  source 1..99: mol_type = other RNA
aaatacagaggaaattaagcatttaagtacctgtaaggaaatttacagatctacttaaacaaggctttatgccgaaatcaaggacatcgacggatgtcc

SEQ ID NO: 488 - RNA, 99 nt, synthetic construct
  source 1..99: mol_type = other RNA
cctccctggtctcagccaccatttaagtacctgtaaggaaatttacagatctacttaaacaaggctttatgccgaaatcaaggacatcgacggatgtcc

SEQ ID NO: 489 - RNA, 99 nt, synthetic construct
  source 1..99: mol_type = other RNA
tctcaaacaaatgtgtcacaatttaagtacctgtaaggaaatttacagatctacttaaacaaggctttatgccgaaatcaaggacatcgacggatgtcc

SEQ ID NO: 490 - RNA, 99 nt, synthetic construct
  source 1..99: mol_type = other RNA
tttgtacaagatccgcagacatttaagtacctgtaaggaaatttacagatctacttaaacaaggctttatgccgaaatcaaggacatcgacggatgtcc

SEQ ID NO: 491 - RNA, 99 nt, synthetic construct
  source 1..99: mol_type = other RNA
ggcaactcagaagcaggtgaatttaagtacctgtaaggaaatttacagatctacttaaacaaggctttatgccgaaatcaaggacatcgacggatgtcc

SEQ ID NO: 492 - RNA, 99 nt, synthetic construct
  source 1..99: mol_type = other RNA
aagtaggttcatctttctccatttaagtacctgtaaggaaatttacagatctacttaaacaaggctttatgccgaaatcaaggacatcgacggatgtcc

SEQ ID NO: 493 - RNA, 99 nt, synthetic construct
  source 1..99: mol_type = other RNA
catcttgaatctgttccatcatttaagtacctgtaaggaaatttacagatctacttaaacaaggctttatgccgaaatcaaggacatcgacggatgtcc

SEQ ID NO: 494 - RNA, 99 nt, synthetic construct
  source 1..99: mol_type = other RNA
tgctcccggagccaaggctgatttaagtacctgtaaggaaatttacagatctacttaaacaaggctttatgccgaaatcaaggacatcgacggatgtcc

SEQ ID NO: 495 - RNA, 99 nt, synthetic construct
  source 1..99: mol_type = other RNA
tggagtaagcgcctgaggtgatttaagtacctgtaaggaaatttacagatctacttaaacaaggctttatgccgaaatcaaggacatcgacggatgtcc

SEQ ID NO: 496 - RNA, 99 nt, synthetic construct
  source 1..99: mol_type = other RNA
cttggtggataccctgaagtatttaagtacctgtaaggaaatttacagatctacttaaacaaggctttatgccgaaatcaaggacatcgacggatgtcc

SEQ ID NO: 497 - RNA, 79 nt, synthetic construct
  source 1..79: mol_type = other RNA
atttaagtacctagtaggaaattattagatctacttaaacaaggctttatgccgaaatcaaggacaccgacgggtgtcc

SEQ ID NO: 498 - RNA, 61 nt, synthetic construct
  source 1..61: mol_type = other RNA
atttaagtacccggtgagaaattatcttcggacctatttaaacaaggctttatgccgaaag

SEQ ID NO: 499 - protein, 1104 aa, synthetic construct
MGLRFGIDIGVASVGWAVVNDEYEVVESGANLFPSADASKNVDRRTFRQMKRVHRRRKTRVSDFDKLWKNHGRKLPESNPEEVLELRVKGLTQALTEEELYAVLKNALLHRGISYLDDAGDEAVSGRSDYERGIMLNRQALDENMHPCEIQFKRLKEYGSYRGNLTIDVDGEKVMLSNVFTTGAYRKEIFEILERQQESHAFLTDDFVENYLKIFSRKREYYVGPGNEKSRTDYGKYTTRIDAETGQYVTEDNIFEKLIGKCSVYKDLTRAAGATYTAQEFNLLNDLNNLTVNGRKLEKEEKVQIIEEVKTANTVNMRKILKRVIGEELETLTGARIDKKEKELFHHFEQYNAIRKAFEKQGWDISELSREDLDSIGDILTLNTERESILGGFQREGLTLSEEKKDCLIALRKKSGALFSKWQSFSLKIMQELIPELYAQPKNQMQLLTDMGVFKTNQEKFAEYHQIPDEMILENIYNPVVSRSVRITVKVLNALVKKYGNPSQIVIEMPRDNSEEEEKKRTKEIQAKNEKELEGIVKRISEEYGIKITEQDFHQHKKLVLKLKLWQEQDGICVYSGRAISIQDLLKNPYLFEIDAIIPKSISFDDSRTNKVLVYRTENQEKRNQTPYLYLKNLRRSWGYDEYRSYVLELKRKGLIPKAKLDKLLFTEDITKIEVLKGFLSRNLNDTRYASRMVLNILQSYFKAKKADTTIRVVRGSFTHQLRNVMRLEKDREESYAHHAVDAMLMCYSQMGFDAYHARQKEIIDFETGEILDENKWNQIMSDKAYDELMYQNQWMRIRANIQKAEKEVKYWHKRDSKPNRGLCNQTIRGTREKDGATMKVNKLNIYEPSGWAKLKKMIEQGKQERFLMYLHDRKTWDDMLRIIEEYQDAANPFVEYEKETGDYLRKYAKKHNGPRIRQLKYLDGEVGSCIDISHKYGHEQGSRKVILESLNPYRMDVYYHAGKKAYFFVGVKYADLKFEKGRYILNEEAYQRALVSEKLIAASQNRFDLEELGYRFVLSFYRNEIIEYEKDGEYYTERFLSRTMPKQKNYIETKPMDAPKFEFVDRHFVGLRKTKSVKKIRLDILGNRFYCSQEKFTLKVDVV

SEQ ID NO: 500-501 - skipped (2 entries)

SEQ ID NO: 502 - protein, 8 aa, synthetic construct
  VARIANT 3: note = Any naturally occurring amino acid
  VARIANT 4: note = W, F, or Y
  VARIANT 5: note = Any naturally occurring amino acid
  VARIANT 6: note = Any naturally occurring amino acid
KRXXXXAF

SEQ ID NO: 503 - skipped

SEQ ID NO: 504 - protein, 6 aa, synthetic construct
  VARIANT 5: note = K or R
  VARIANT 6: note = W, F, or Y
LGKRXX

SEQ ID NO: 505-511 - skipped (7 entries)

SEQ ID NO: 512 - protein, 16 aa, synthetic construct
APKKKRKVGIHGVPAA

SEQ ID NO: 513 - protein, 5 aa, synthetic construct
GSGGS

SEQ ID NO: 514 - protein, 6 aa, synthetic construct
GGSGGS

SEQ ID NO: 515 - protein, 4 aa, synthetic construct
GGGS

SEQ ID NO: 516 - protein, 4 aa, synthetic construct
GGSG

SEQ ID NO: 517 - protein, 5 aa, synthetic construct
GGSGG

SEQ ID NO: 518 - protein, 5 aa, synthetic construct
GSGSG

SEQ ID NO: 519 - protein, 5 aa, synthetic construct
GSGGG

SEQ ID NO: 520 - protein, 5 aa, synthetic construct
GGGSG

SEQ ID NO: 521 - protein, 5 aa, synthetic construct
GSSSG

SEQ ID NO: 522 - protein, 6 aa, synthetic construct
HHHHHH

SEQ ID NO: 523 - DNA, 20 bp, synthetic construct
  source 1..20: mol_type = other DNA
  misc_difference 1..20: note = This sequence may encompass 5-20 nucleotides
aaaaaaaaaaaaaaaaaaaa

SEQ ID NO: 524 - DNA, 20 bp, synthetic construct
  source 1..20: mol_type = other DNA
  misc_difference 1..20: note = This sequence may encompass 5-20 nucleotides
tttttttttttttttttttt

SEQ ID NO: 525 - DNA, 20 bp, synthetic construct
  source 1..20: mol_type = other DNA
  misc_difference 1..20: note = This sequence may encompass 5-20 nucleotides
cccccccccccccccccccc

SEQ ID NO: 526 - DNA, 20 bp, synthetic construct
  source 1..20: mol_type = other DNA
  misc_difference 1..20: note = This sequence may encompass 5-20 nucleotides
gggggggggggggggggggg

(19) United States
(12) Patent Application Publication
(10) Pub. No.: US 2024/0384247 A1
(43) Pub. Date: Nov. 21, 2024

(54) EFFECTOR PROTEINS AND USES THEREOF

(71) Applicant: Mammoth Biosciences, Inc., Brisbane, CA (US)

(72) Inventors: Aaron DELOUGHERY, San Francisco, CA (US); Benjamin RAUCH, San Francisco, CA (US); Clarissa Oriel RHINES, San Mateo, CA (US); Stepan TYMOSHENKO, Sacramento, CA (US); Fnu YUNANDA, Daly City, CA (US); Lucas Benjamin HARRINGTON, San Francisco, CA (US)

(21) Appl. No.: 18/669,081

(22) Filed: May 20, 2024

Related U.S. Application Data
(63) Continuation of application No. PCT/US2022/080266, filed on Nov. 21, 2022.
(60) Provisional application No. 63/282,118, filed on Nov. 22, 2021.
(60) Provisional application No. 63/332,938, filed on Apr. 20, 2022.

(51) Int. Cl. C12N 9/22; A61K 9/51; A61K 38/00; C12N 15/11

(52) U.S. Cl. CPC C12N 9/22; A61K 9/5123; C12N 15/11; A61K 38/00; C12N 2310/16; C12N 2310/20; C12Y 207/07049

(57) ABSTRACT

Provided herein are compositions, systems, and methods comprising effector proteins and uses thereof. These effector proteins may be characterized as CRISPR-associated (Cas) proteins. Various compositions, systems, and methods of the present disclosure may leverage the activities of these effector proteins for the modification, detection, and engineering of nucleic acids.

### CROSS REFERENCE TO RELATED APPLICATIONS

This application is a continuation of International Patent Application No. PCT/US2022/080266, filed Nov. 21, 2022, which claims the benefit of priority of U.S. Provisional Application No. 63/282,118, filed Nov. 22, 2021, and U.S. Provisional Application No. 63/332,938, filed Apr. 20, 2022, the disclosures of which are incorporated herein by reference in their entirety.

### SEQUENCE LISTING

The instant application contains a Sequence Listing, which has been submitted via Patent Center. The Sequence Listing titled 203477-706301_US_SL.xml, which was created on May 10, 2024, and is 558,354 bytes in size, is hereby incorporated by reference in its entirety.

### FIELD

The present disclosure relates generally to compositions of effector proteins and guide nucleic acids, and methods and systems of using such compositions.

### BACKGROUND

Programmable nucleases are proteins that bind and cleave nucleic acids in a sequence-specific manner. A programmable nuclease may bind a target region of a nucleic acid and cleave the nucleic acid within the target region or at a position adjacent to the target region. In some embodiments, a programmable nuclease is activated when it binds a target region of a nucleic acid to cleave regions of the nucleic acid that are near, but not adjacent to the target region. A programmable nuclease, such as a CRISPR-associated (Cas) protein, may be coupled to a guide nucleic acid that imparts activity or sequence selectivity to the programmable nuclease. In general, guide nucleic acids comprise a CRISPR RNA (crRNA) that is at least partially complementary to a target nucleic acid. In some cases, guide nucleic acids comprise a trans-activating crRNA (tracrRNA) sequence, at least a portion of which interacts with the programmable nuclease. In some cases, a tracrRNA is provided separately from the crRNA and hybridizes to a portion of the crRNA that does not hybridize to the target nucleic acid. In other cases, the tracrRNA sequence and crRNA are linked as a single guide RNA (sgRNA).

Programmable nucleases may cleave nucleic acids, including single stranded RNA (ssRNA), double stranded DNA (dsDNA), and single-stranded DNA (ssDNA). Programmable nucleases may provide cis cleavage activity, trans cleavage activity, nickase activity, or a combination thereof. Cis cleavage activity is cleavage of a target nucleic acid that is hybridized to a guide nucleic acid, wherein cleavage occurs within or directly adjacent to the region of the target nucleic acid that is hybridized to guide RNA. Trans cleavage activity includes cleavage of DNA or RNA that is near, but not hybridized to the guide RNA. Trans cleavage activity may be triggered by the hybridization of guide RNA to the target nucleic acid. Nickase activity is the selective cleavage of one strand of a dsDNA molecule.

Programmable nucleases may be modified to have reduced nuclease or nickase activity relative to its unmodified version, but retain their sequence selectivity. For instance, amino acid residues of the programmable nuclease that impart catalytic activity to the programmable nuclease may be substituted with an alternative amino acid that does not impart catalytic activity to the programmable nuclease.

While certain programmable nucleases may be used to edit and detect nucleic acid molecules in a sequence specific manner, challenging biological and sample conditions (e.g., high viscosity, metal chelating) may limit their accuracy and effectiveness. There is thus a need for systems and methods that employ programmable nucleases having specificity and efficiency across a wide range of biological and sample conditions.

### SUMMARY

The present disclosure provides compositions, systems, and methods comprising effector proteins and uses thereof. Compositions, systems, and methods disclosed herein leverage nucleic acid modifying activities (e.g., cis cleavage activity and trans cleavage activity) of these effector proteins for the modification, detection, and engineering of target nucleic acids.

### Certain Embodiments

In some aspects, provided herein are compositions comprising an effector protein or a nucleic acid encoding the effector protein, and a guide nucleic acid or a nucleic acid encoding the guide nucleic acid, wherein the effector protein comprises an amino acid sequence that is at least 75%, at least 80%, at least 85%, at least 90%, at least 95%, at least 98%, at least 99%, or 100% identical to any one of the effector proteins described herein (e.g., SEQ ID NOS: 1-78 and 499).

In some aspects, provided herein are compositions comprising an effector protein or a nucleic acid encoding the effector protein, and a guide nucleic acid or a nucleic acid encoding the guide nucleic acid, wherein the amino acid sequence of the effector protein is at least 75%, at least 80%, at least 85%, at least 90%, at least 95%, at least 98%, at least 99%, or 100% identical to any one of the effector proteins described herein (e.g., SEQ ID NOS: 1-78 and 499).

In some aspects, provided herein are compositions comprising an effector protein or a nucleic acid encoding the effector protein, and a guide nucleic acid or a nucleic acid encoding the guide nucleic acid, wherein the effector protein comprises about 100, about 120, about 140, about 160, about 180, about 200, about 220, about 240, about 260, about 280, about 300, about 320, about 340, about 360, about 380, about 400, about 420, about 440, about 460, about 480, about 500, about 520, about 540, about 560, about 580, about 600, about 620, about 640, about 660, about 680, about 700, about 720, about 740, about 760, about 780, about 800, about 820, about 840, about 860, about 880, about 900, about 920, about 940, about 960, about 980, about 1000, about 1020, about 1040, about 1060, about 1080, about 1100, about 1120, about 1140, about 1160, about 1180, about 1200, about 1220, about 1240, about 1260, about 1280, about 1300, about 1320, about 1340, about 1360, about 1380, or about 1400 contiguous amino acids of a sequence described herein (e.g., SEQ ID NOS: 1-78 and 499).

In some aspects, provided herein are compositions comprising an effector protein or a nucleic acid encoding the effector protein, and a guide nucleic acid or a nucleic acid encoding the guide nucleic acid, wherein the effector protein comprises the amino acid sequence located at positions 1-100, 150-250, 101-200, 250-350, 201-300, 350-450, 301-400, 350-450, 401-500, 450-550, 501-600, 550-650, 601-700, 650-750, 701-800, 750-850, 801-900, 850-950, 901-1000, 950-1050, 1001-1100, 1050-1150, 1101-1200, 1150-1250, 1201-1300, 1250-1350, 1301-1400, 1350-1450, 1401-1500, 1450-1550, 1501-1600, 1550-1650, 1601-1700, 1650-1750, 1701-1800, 1850-1950, or 1801-1900 of a sequence described herein (e.g., SEQ ID NOS: 1-78 and 499).

In some aspects, provided herein are compositions comprising an effector protein or a nucleic acid encoding the effector protein, and a guide nucleic acid or a nucleic acid encoding the guide nucleic acid, wherein the effector protein comprises an amino acid sequence that is at least 90%, at least 95%, or 100% identical to a portion of a sequence described herein (e.g., SEQ ID NOS: 1-78 and 499), wherein the portion of the sequence is about 30%, about 40% about 50%, about 60%, about 70%, about 80%, or about 90% of a sequence described herein (e.g., SEQ ID NOS: 1-78 and 499).

In some embodiments, at least a portion of the guide nucleic acid binds the effector protein. In some embodiments, the guide nucleic acid comprises a crRNA. In some embodiments, the guide nucleic acid comprises a tracrRNA sequence. In some embodiments, the guide nucleic acid does not comprise a tracrRNA. In some embodiments, the guide nucleic acid comprises a crRNA covalently linked to a tracrRNA sequence. In some embodiments, the guide nucleic acid comprises a first sequence and a second sequence, wherein the first sequence is heterologous with the second sequence. In some embodiments, the first sequence comprises at least five amino acids and the second sequence comprises at least five amino acids.

In some embodiments, at least one of the effector protein, the guide nucleic acid, and the combination thereof, are not naturally occurring. In some embodiments, at least one of the effector protein and the guide nucleic acid is recombinant or engineered.

In some embodiments, the guide nucleic acid comprises a sequence that hybridizes to a target sequence of a target nucleic acid, and wherein the target nucleic acid comprises a protospacer adjacent motif (PAM). In some embodiments, the PAM is located within 1, 5, 10, 15, 20, 40, 60, 80, or 100 nucleotides of the 5′ end of the target sequence. In some embodiments, the PAM comprises a nucleotide sequence that is at least 80%, at least 85%, at least 90%, at least 95%, at least 98% or 100% identical to a nucleotide sequence selected from SEQ ID NOS: 79-122. In some embodiments, the PAM comprises a nucleotide sequence that is at least 90% identical to SEQ ID NO: 79. In some embodiments, the PAM comprises a nucleotide sequence that is at least 90% identical to SEQ ID NO: 80. In some embodiments, the PAM comprises a nucleotide sequence that is at least 90% identical to SEQ ID NO: 81. In some embodiments, the PAM comprises a nucleotide sequence that is at least 90% identical to SEQ ID NO: 82. In some embodiments, the PAM comprises a nucleotide sequence that is at least 90% identical to SEQ ID NO: 83. In some embodiments, the PAM comprises a nucleotide sequence that is at least 90% identical to SEQ ID NO: 84. In some embodiments, the PAM comprises a nucleotide sequence that is at least 90% identical to SEQ ID NO: 85. In some embodiments, the PAM comprises a nucleotide sequence that is at least 90% identical to SEQ ID NO: 86. In some embodiments, the PAM comprises a nucleotide sequence that is at least 90% identical to SEQ ID NO: 87. In some embodiments, the PAM comprises a nucleotide sequence that is at least 90% identical to SEQ ID NO: 88. In some embodiments, the PAM comprises a nucleotide sequence that is at least 90% identical to SEQ ID NO: 89. In some embodiments, the PAM comprises a nucleotide sequence that is at least 90% identical to SEQ ID NO: 90. In some embodiments, the PAM comprises a nucleotide sequence that is at least 90% identical to SEQ ID NO: 91. In some embodiments, the PAM comprises a nucleotide sequence that is at least 90% identical to SEQ ID NO: 92. In some embodiments, the PAM comprises a nucleotide sequence that is at least 90% identical to SEQ ID NO: 93. In some embodiments, the PAM comprises a nucleotide sequence that is at least 90% identical to SEQ ID NO: 94. In some embodiments, the PAM comprises a nucleotide sequence that is at least 90% identical to SEQ ID NO: 95. In some embodiments, the PAM comprises a nucleotide sequence that is at least 90% identical to SEQ ID NO: 96. In some embodiments, the PAM comprises a nucleotide sequence that is at least 90% identical to SEQ ID NO: 97. In some embodiments, the PAM comprises a nucleotide sequence that is at least 90% identical to SEQ ID NO: 98. In some embodiments, the PAM comprises a nucleotide sequence that is at least 90% identical to SEQ ID NO: 99. In some embodiments, the PAM comprises a nucleotide sequence that is at least 90% identical to SEQ ID NO: 100. In some embodiments, the PAM comprises a nucleotide sequence that is at least 90% identical to SEQ ID NO: 101. In some embodiments, the PAM comprises a nucleotide sequence that is at least 90% identical to SEQ ID NO: 102. In some embodiments, the PAM comprises a nucleotide sequence that is at least 90% identical to SEQ ID NO: 103. In some embodiments, the PAM comprises a nucleotide sequence that is at least 90% identical to SEQ ID NO: 104. In some embodiments, the PAM comprises a nucleotide sequence that is at least 90% identical to SEQ ID NO: 105. In some embodiments, the PAM comprises a nucleotide sequence that is at least 90% identical to SEQ ID NO: 106. In some embodiments, the PAM comprises a nucleotide sequence that is at least 90% identical to SEQ ID NO: 107. In some embodiments, the PAM comprises a nucleotide sequence that is at least 90% identical to SEQ ID NO: 108. In some embodiments, the PAM comprises a nucleotide sequence that is at least 90% identical to SEQ ID NO: 109. In some embodiments, the PAM comprises a nucleotide sequence that is at least 90% identical to SEQ ID NO: 110. In some embodiments, the PAM comprises a nucleotide sequence that is at least 90% identical to SEQ ID NO: 111. In some embodiments, the PAM comprises a nucleotide sequence that is at least 90% identical to SEQ ID NO: 112. In some embodiments, the PAM comprises a nucleotide sequence that is at least 90% identical to SEQ ID NO: 113. In some embodiments, the PAM comprises a nucleotide sequence that is at least 90% identical to SEQ ID NO: 114. In some embodiments, the PAM comprises a nucleotide sequence that is at least 90% identical to SEQ ID NO: 115. In some embodiments, the PAM comprises a nucleotide sequence that is at least 90% identical to SEQ ID NO: 116. In some embodiments, the PAM comprises a nucleotide sequence that is at least 90% identical to SEQ ID NO: 117. In some embodiments, the PAM comprises a nucleotide sequence that is at least 90% identical to SEQ ID NO: 118. In some embodiments, the PAM comprises a nucleotide sequence that is at least 90% identical to SEQ ID NO: 119. In some embodiments, the PAM comprises a nucleotide sequence that is at least 90% identical to SEQ ID NO: 120. In some embodiments, the PAM comprises a nucleotide sequence that is at least 90% identical to SEQ ID NO: 121. In some embodiments, the PAM comprises a nucleotide sequence that is at least 90% identical to SEQ ID NO: 122.

In some embodiments, the guide nucleic acid comprises a repeat sequence that comprises at least 8, at least 9, at least 10, at least 11, at least 12, at least 13, at least 14, at least 15, at least 16, at least 17, at least 18, at least 19, or at least 20 contiguous nucleotides of a nucleotide sequence described herein (e.g., SEQ ID NOS: 123-165). In some embodiments, the guide nucleic acid comprises a repeat sequence that comprises a nucleotide sequence that is at least 80%, at least 85%, at least 90%, at least 95%, at least 98% or 100% identical to a nucleotide sequence described herein (e.g., SEQ ID NOS: 123-165). In some embodiments, the effector protein comprises an amino acid sequence that is at least 90% identical to SEQ ID NO: 1, and the guide nucleic acid comprises a repeat sequence that comprises a nucleotide sequence that is at least 90% identical to SEQ ID NO: 123. In some embodiments, the effector protein comprises an amino acid sequence that is at least 90% identical to SEQ ID NO: 2, and the guide nucleic acid comprises a repeat sequence that comprises a nucleotide sequence that is at least 90% identical to SEQ ID NO: 124. In some embodiments, the effector protein comprises an amino acid sequence that is at least 90% identical to SEQ ID NO: 5, and the guide nucleic acid comprises a repeat sequence that comprises a nucleotide sequence that is at least 90% identical to SEQ ID NO: 125. In some embodiments, the effector protein comprises an amino acid sequence that is at least 90% identical to SEQ ID NO: 9, and the guide nucleic acid comprises a repeat sequence that comprises a nucleotide sequence that is at least 90% identical to SEQ ID NO: 126. In some embodiments, the effector protein comprises an amino acid sequence that is at least 90% identical to SEQ ID NO: 10, and the guide nucleic acid comprises a repeat sequence that comprises a nucleotide sequence that is at least 90% identical to SEQ ID NO: 125. In some embodiments, the effector protein comprises an amino acid sequence that is at least 90% identical to SEQ ID NO: 11, and the guide nucleic acid comprises a repeat sequence that comprises a nucleotide sequence that is at least 90% identical to SEQ ID NO: 127. In some embodiments, the effector protein comprises an amino acid sequence that is at least 90% identical to SEQ ID NO: 14, and the guide nucleic acid comprises a repeat sequence that comprises a nucleotide sequence that is at least 90% identical to SEQ ID NO: 125. In some embodiments, the effector protein comprises an amino acid sequence that is at least 90% identical to SEQ ID NO: 15, and the guide nucleic acid comprises a repeat sequence that comprises a nucleotide sequence that is at least 90% identical to SEQ ID NO: 125. In some embodiments, the effector protein comprises an amino acid sequence that is at least 90% identical to SEQ ID NO: 16, and the guide nucleic acid comprises a repeat sequence that comprises a nucleotide sequence that is at least 90% identical to SEQ ID NO: 128. In some embodiments, the effector protein comprises an amino acid sequence that is at least 90% identical to SEQ ID NO: 16, and the guide nucleic acid comprises a repeat sequence that comprises a nucleotide sequence that is at least 90% identical to SEQ ID NO: 129. In some embodiments, the effector protein comprises an amino acid sequence that is at least 90% identical to SEQ ID NO: 17, and the guide nucleic acid comprises a repeat sequence that comprises a nucleotide sequence that is at least 90% identical to SEQ ID NO: 130. In some embodiments, the effector protein comprises an amino acid sequence that is at least 90% identical to SEQ ID NO: 17, and the guide nucleic acid comprises a repeat sequence that comprises a nucleotide sequence that is at least 90% identical to SEQ ID NO: 131. In some embodiments, the effector protein comprises an amino acid sequence that is at least 90% identical to SEQ ID NO: 20, and the guide nucleic acid comprises a repeat sequence that comprises a nucleotide sequence that is at least 90% identical to SEQ ID NO: 132. In some embodiments, the effector protein comprises an amino acid sequence that is at least 90% identical to SEQ ID NO: 20, and the guide nucleic acid comprises a repeat sequence that comprises a nucleotide sequence that is at least 90% identical to SEQ ID NO: 133. In some embodiments, the effector protein comprises an amino acid sequence that is at least 90% identical to SEQ ID NO: 21, and the guide nucleic acid comprises a repeat sequence that comprises a nucleotide sequence that is at least 90% identical to SEQ ID NO: 125. In some embodiments, the effector protein comprises an amino acid sequence that is at least 90% identical to SEQ ID NO: 22, and the guide nucleic acid comprises a repeat sequence that comprises a nucleotide sequence that is at least 90% identical to SEQ ID NO: 134. In some embodiments, the effector protein comprises an amino acid sequence that is at least 90% identical to SEQ ID NO: 23, and the guide nucleic acid comprises a repeat sequence that comprises a nucleotide sequence that is at least 90% identical to SEQ ID NO: 123. In some embodiments, the effector protein comprises an amino acid sequence that is at least 90% identical to SEQ ID NO: 29, and the guide nucleic acid comprises a repeat sequence that comprises a nucleotide sequence that is at least 90% identical to SEQ ID NO: 135. In some embodiments, the effector protein comprises an amino acid sequence that is at least 90% identical to SEQ ID NO: 31, and the guide nucleic acid comprises a repeat sequence that comprises a nucleotide sequence that is at least 90% identical to SEQ ID NO: 136. In some embodiments, the effector protein comprises an amino acid sequence that is at least 90% identical to SEQ ID NO: 32, and the guide nucleic acid comprises a repeat sequence that comprises a nucleotide sequence that is at least 90% identical to SEQ ID NO: 137. In some embodiments, the effector protein comprises an amino acid sequence that is at least 90% identical to SEQ ID NO: 33, and the guide nucleic acid comprises a repeat sequence that comprises a nucleotide sequence that is at least 90% identical to SEQ ID NO: 137. In some embodiments, the effector protein comprises an amino acid sequence that is at least 90% identical to SEQ ID NO: 34, and the guide nucleic acid comprises a repeat sequence that comprises a nucleotide sequence that is at least 90% identical to SEQ ID NO: 138. In some embodiments, the effector protein comprises an amino acid sequence that is at least 90% identical to SEQ ID NO: 36, and the guide nucleic acid comprises a repeat sequence that comprises a nucleotide sequence that is at least 90% identical to SEQ ID NO: 139. In some embodiments, the effector protein comprises an amino acid sequence that is at least 90% identical to SEQ ID NO: 37, and the guide nucleic acid comprises a repeat sequence that comprises a nucleotide sequence that is at least 90% identical to SEQ ID NO: 136. In some embodiments, the effector protein comprises an amino acid sequence that is at least 90% identical to SEQ ID NO: 38, and the guide nucleic acid comprises a repeat sequence that comprises a nucleotide sequence that is at least 90% identical to SEQ ID NO: 140. In some embodiments, the effector protein comprises an amino acid sequence that is at least 90% identical to SEQ ID NO: 40, and the guide nucleic acid comprises a repeat sequence that comprises a nucleotide sequence that is at least 90% identical to SEQ ID NO: 141. In some embodiments, the effector protein comprises an amino acid sequence that is at least 90% identical to SEQ ID NO: 41, and the guide nucleic acid comprises a repeat sequence that comprises a nucleotide sequence that is at least 90% identical to SEQ ID NO: 142. In some embodiments, the effector protein comprises an amino acid sequence that is at least 90% identical to SEQ ID NO: 43, and the guide nucleic acid comprises a repeat sequence that comprises a nucleotide sequence that is at least 90% identical to SEQ ID NO: 136. In some embodiments, the effector protein comprises an amino acid sequence that is at least 90% identical to SEQ ID NO: 44, and the guide nucleic acid comprises a repeat sequence that comprises a nucleotide sequence that is at least 90% identical to SEQ ID NO: 136. In some embodiments, the effector protein comprises an amino acid sequence that is at least 90% identical to SEQ ID NO: 45, and the guide nucleic acid comprises a repeat sequence that comprises a nucleotide sequence that is at least 90% identical to SEQ ID NO: 137. In some embodiments, the effector protein comprises an amino acid sequence that is at least 90% identical to SEQ ID NO: 46, and the guide nucleic acid comprises a repeat sequence that comprises a nucleotide sequence that is at least 90% identical to SEQ ID NO: 143. In some embodiments, the effector protein comprises an amino acid sequence that is at least 90% identical to SEQ ID NO: 47, and the guide nucleic acid comprises a repeat sequence that comprises a nucleotide sequence that is at least 90% identical to SEQ ID NO: 144. In some embodiments, the effector protein comprises an amino acid sequence that is at least 90% identical to SEQ ID NO: 47, and the guide nucleic acid comprises a repeat sequence that comprises a nucleotide sequence that is at least 90% identical to SEQ ID NO: 145. In some embodiments, the effector protein comprises an amino acid sequence that is at least 90% identical to SEQ ID NO: 49, and the guide nucleic acid comprises a repeat sequence that comprises a nucleotide sequence that is at least 90% identical to SEQ ID NO: 146. In some embodiments, the effector protein comprises an amino acid sequence that is at least 90% identical to SEQ ID NO: 51, and the guide nucleic acid comprises a repeat sequence that comprises a nucleotide sequence that is at least 90% identical to SEQ ID NO: 147. In some embodiments, the effector protein comprises an amino acid sequence that is at least 90% identical to SEQ ID NO: 53, and the guide nucleic acid comprises a repeat sequence that comprises a nucleotide sequence that is at least 90% identical to SEQ ID NO: 148. In some embodiments, the effector protein comprises an amino acid sequence that is at least 90% identical to SEQ ID NO: 53, and the guide nucleic acid comprises a repeat sequence that comprises a nucleotide sequence that is at least 90% identical to SEQ ID NO: 149. In some embodiments, the effector protein comprises an amino acid sequence that is at least 90% identical to SEQ ID NO: 54, and the guide nucleic acid comprises a repeat sequence that comprises a nucleotide sequence that is at least 90% identical to SEQ ID NO: 137. In some embodiments, the effector protein comprises an amino acid sequence that is at least 90% identical to SEQ ID NO: 55, and the guide nucleic acid comprises a repeat sequence that comprises a nucleotide sequence that is at least 90% identical to SEQ ID NO: 150. In some embodiments, the effector protein comprises an amino acid sequence that is at least 90% identical to SEQ ID NO: 56, and the guide nucleic acid comprises a repeat sequence that comprises a nucleotide sequence that is at least 90% identical to SEQ ID NO: 147. In some embodiments, the effector protein comprises an amino acid sequence that is at least 90% identical to SEQ ID NO: 57, and the guide nucleic acid comprises a repeat sequence that comprises a nucleotide sequence that is at least 90% identical to SEQ ID NO: 151. In some embodiments, the effector protein comprises an amino acid sequence that is at least 90% identical to SEQ ID NO: 58, and the guide nucleic acid comprises a repeat sequence that comprises a nucleotide sequence that is at least 90% identical to SEQ ID NO: 152. In some embodiments, the effector protein comprises an amino acid sequence that is at least 90% identical to SEQ ID NO: 59, and the guide nucleic acid comprises a repeat sequence that comprises a nucleotide sequence that is at least 90% identical to SEQ ID NO: 153. In some embodiments, the effector protein comprises an amino acid sequence that is at least 90% identical to SEQ ID NO: 59, and the guide nucleic acid comprises a repeat sequence that comprises a nucleotide sequence that is at least 90% identical to SEQ ID NO: 154. In some embodiments, the effector protein comprises an amino acid sequence that is at least 90% identical to SEQ ID NO: 60, and the guide nucleic acid comprises a repeat sequence that comprises a nucleotide sequence that is at least 90% identical to SEQ ID NO: 155. In some embodiments, the effector protein comprises an amino acid sequence that is at least 90% identical to SEQ ID NO: 62, and the guide nucleic acid comprises a repeat sequence that comprises a nucleotide sequence that is at least 90% identical to SEQ ID NO: 137. In some embodiments, the effector protein comprises an amino acid sequence that is at least 90% identical to SEQ ID NO: 63, and the guide nucleic acid comprises a repeat sequence that comprises a nucleotide sequence that is at least 90% identical to SEQ ID NO: 125. In some embodiments, the effector protein comprises an amino acid sequence that is at least 90% identical to SEQ ID NO: 65, and the guide nucleic acid comprises a repeat sequence that comprises a nucleotide sequence that is at least 90% identical to SEQ ID NO: 156. In some embodiments, the effector protein comprises an amino acid sequence that is at least 90% identical to SEQ ID NO: 66, and the guide nucleic acid comprises a repeat sequence that comprises a nucleotide sequence that is at least 90% identical to SEQ ID NO: 157. In some embodiments, the effector protein comprises an amino acid sequence that is at least 90% identical to SEQ ID NO: 68, and the guide nucleic acid comprises a repeat sequence that comprises a nucleotide sequence that is at least 90% identical to SEQ ID NO: 158. In some embodiments, the effector protein comprises an amino acid sequence that is at least 90% identical to SEQ ID NO: 71, and the guide nucleic acid comprises a repeat sequence that comprises a nucleotide sequence that is at least 90% identical to SEQ ID NO: 159. In some embodiments, the effector protein comprises an amino acid sequence that is at least 90% identical to SEQ ID NO: 71, and the guide nucleic acid comprises a repeat sequence that comprises a nucleotide sequence that is at least 90% identical to SEQ ID NO: 160. In some embodiments, the effector protein comprises an amino acid sequence that is at least 90% identical to SEQ ID NO: 73, and the guide nucleic acid comprises a repeat sequence that comprises a nucleotide sequence that is at least 90% identical to SEQ ID NO: 137. In some embodiments, the effector protein comprises an amino acid sequence that is at least 90% identical to SEQ ID NO: 75, and the guide nucleic acid comprises a repeat sequence that comprises a nucleotide sequence that is at least 90% identical to SEQ ID NO: 161. In some embodiments, the effector protein comprises an amino acid sequence that is at least 90% identical to SEQ ID NO: 75, and the guide nucleic acid comprises a repeat sequence that comprises a nucleotide sequence that is at least 90% identical to SEQ ID NO: 162. In some embodiments, the effector protein comprises an amino acid sequence that is at least 90% identical to SEQ ID NO: 76, and the guide nucleic acid comprises a repeat sequence that comprises a nucleotide sequence that is at least 90% identical to SEQ ID NO: 137. In some embodiments, the effector protein comprises an amino acid sequence that is at least 90% identical to SEQ ID NO: 6, and the guide nucleic acid comprises a repeat sequence that comprises a nucleotide sequence that is at least 90% identical to SEQ ID NO: 147. In some embodiments, the effector protein comprises an amino acid sequence that is at least 90% identical to SEQ ID NO: 7, and the guide nucleic acid comprises a repeat sequence that comprises a nucleotide sequence that is at least 90% identical to SEQ ID NO: 163. In some embodiments, the effector protein comprises an amino acid sequence that is at least 90% identical to SEQ ID NO: 25, and the guide nucleic acid comprises a repeat sequence that comprises a nucleotide sequence that is at least 90% identical to SEQ ID NO: 164. In some embodiments, the effector protein comprises an amino acid sequence that is at least 90% identical to SEQ ID NO: 64, and the guide nucleic acid comprises a repeat sequence that comprises a nucleotide sequence that is at least 90% identical to SEQ ID NO: 165.

In some embodiments, the guide nucleic acid comprises at least 8, at least 9, at least 10, at least 11, at least 12, at least 13, at least 14, at least 15, at least 16, at least 17, at least 18, at least 19, or at least 20 contiguous nucleotides of a nucleotide sequence described herein (e.g., SEQ ID NOS: 166-227, 327-425 and 436-498). In some embodiments, the guide nucleic acid comprises at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, or at least 220 contiguous nucleotides of a nucleotide sequence described herein (e.g., SEQ ID NOS: 166-227, 327-425 and 436-498). In some embodiments, the guide nucleic acid comprises a nucleotide sequence that is at least 80%, at least 85%, at least 90%, at least 95%, at least 98% or 100% identical to a nucleotide sequence described herein (e.g., SEQ ID NOS: 166-227, 327-425 and 436-498). In some embodiments, the effector protein comprises an amino acid sequence that is at least 90% identical to SEQ ID NO: 1, and the guide nucleic acid comprises a nucleotide sequence that is at least 90% identical to SEQ ID NO: 166. In some embodiments, the effector protein comprises an amino acid sequence that is at least 90% identical to SEQ ID NO: 2, and the guide nucleic acid comprises a nucleotide sequence that is at least 90% identical to SEQ ID NO: 167. In some embodiments, the effector protein comprises an amino acid sequence that is at least 90% identical to SEQ ID NO: 5, and the guide nucleic acid comprises a nucleotide sequence that is at least 90% identical to SEQ ID NO: 168. In some embodiments, the effector protein comprises an amino acid sequence that is at least 90% identical to SEQ ID NO: 9, and the guide nucleic acid comprises a nucleotide sequence that is at least 90% identical to SEQ ID NO: 169. In some embodiments, the effector protein comprises an amino acid sequence that is at least 90% identical to SEQ ID NO: 10, and the guide nucleic acid comprises a nucleotide sequence that is at least 90% identical to SEQ ID NO: 170. In some embodiments, the effector protein comprises an amino acid sequence that is at least 90% identical to SEQ ID NO: 11, and the guide nucleic acid comprises a nucleotide sequence that is at least 90% identical to SEQ ID NO: 171. In some embodiments, the effector protein comprises an amino acid sequence that is at least 90% identical to SEQ ID NO: 14, and the guide nucleic acid comprises a nucleotide sequence that is at least 90% identical to SEQ ID NO: 172. In some embodiments, the effector protein comprises an amino acid sequence that is at least 90% identical to SEQ ID NO: 15, and the guide nucleic acid comprises a nucleotide sequence that is at least 90% identical to SEQ ID NO: 173. In some embodiments, the effector protein comprises an amino acid sequence that is at least 90% identical to SEQ ID NO: 16, and the guide nucleic acid comprises a nucleotide sequence that is at least 90% identical to SEQ ID NO: 174. In some embodiments, the effector protein comprises an amino acid sequence that is at least 90% identical to SEQ ID NO: 16, and the guide nucleic acid comprises a nucleotide sequence that is at least 90% identical to SEQ ID NO: 175. In some embodiments, the effector protein comprises an amino acid sequence that is at least 90% identical to SEQ ID NO: 17, and the guide nucleic acid comprises a nucleotide sequence that is at least 90% identical to SEQ ID NO: 176. In some embodiments, the effector protein comprises an amino acid sequence that is at least 90% identical to SEQ ID NO: 17, and the guide nucleic acid comprises a nucleotide sequence that is at least 90% identical to SEQ ID NO: 177. In some embodiments, the effector protein comprises an amino acid sequence that is at least 90% identical to SEQ ID NO: 20, and the guide nucleic acid comprises a nucleotide sequence that is at least 90% identical to SEQ ID NO: 178. In some embodiments, the effector protein comprises an amino acid sequence that is at least 90% identical to SEQ ID NO: 20, and the guide nucleic acid comprises a nucleotide sequence that is at least 90% identical to SEQ ID NO: 179. In some embodiments, the effector protein comprises an amino acid sequence that is at least 90% identical to SEQ ID NO: 21, and the guide nucleic acid comprises a nucleotide sequence that is at least 90% identical to SEQ ID NO: 180. In some embodiments, the effector protein comprises an amino acid sequence that is at least 90% identical to SEQ ID NO: 22, and the guide nucleic acid comprises a nucleotide sequence that is at least 90% identical to SEQ ID NO: 181. In some embodiments, the effector protein comprises an amino acid sequence that is at least 90% identical to SEQ ID NO: 23, and the guide nucleic acid comprises a nucleotide sequence that is at least 90% identical to SEQ ID NO: 182. In some embodiments, the effector protein comprises an amino acid sequence that is at least 90% identical to SEQ ID NO: 29, and the guide nucleic acid comprises a nucleotide sequence that is at least 90% identical to SEQ ID NO: 183. In some embodiments, the effector protein comprises an amino acid sequence that is at least 90% identical to SEQ ID NO: 31, and the guide nucleic acid comprises a nucleotide sequence that is at least 90% identical to SEQ ID NO: 184. In some embodiments, the effector protein comprises an amino acid sequence that is at least 90% identical to SEQ ID NO: 32, and the guide nucleic acid comprises a nucleotide sequence that is at least 90% identical to SEQ ID NO: 185. In some embodiments, the effector protein comprises an amino acid sequence that is at least 90% identical to SEQ ID NO: 33, and the guide nucleic acid comprises a nucleotide sequence that is at least 90% identical to SEQ ID NO: 186. In some embodiments, the effector protein comprises an amino acid sequence that is at least 90% identical to SEQ ID NO: 34, and the guide nucleic acid comprises a nucleotide sequence that is at least 90% identical to SEQ ID NO: 187. In some embodiments, the effector protein comprises an amino acid sequence that is at least 90% identical to SEQ ID NO: 36, and the guide nucleic acid comprises a nucleotide sequence that is at least 90% identical to SEQ ID NO: 188. In some embodiments, the effector protein comprises an amino acid sequence that is at least 90% identical to SEQ ID NO: 37, and the guide nucleic acid comprises a nucleotide sequence that is at least 90% identical to SEQ ID NO: 189. In some embodiments, the effector protein comprises an amino acid sequence that is at least 90% identical to SEQ ID NO: 38, and the guide nucleic acid comprises a nucleotide sequence that is at least 90% identical to SEQ ID NO: 190. In some embodiments, the effector protein comprises an amino acid sequence that is at least 90% identical to SEQ ID NO: 38, and the guide nucleic acid comprises a nucleotide sequence that is at least 90% identical to SEQ ID NO: 191. In some embodiments, the effector protein comprises an amino acid sequence that is at least 90% identical to SEQ ID NO: 40, and the guide nucleic acid comprises a nucleotide sequence that is at least 90% identical to SEQ ID NO: 192. In some embodiments, the effector protein comprises an amino acid sequence that is at least 90% identical to SEQ ID NO: 40, and the guide nucleic acid comprises a nucleotide sequence that is at least 90% identical to SEQ ID NO: 193. In some embodiments, the effector protein comprises an amino acid sequence that is at least 90% identical to SEQ ID NO: 41, and the guide nucleic acid comprises a nucleotide sequence that is at least 90% identical to SEQ ID NO: 194. In some embodiments, the effector protein comprises an amino acid sequence that is at least 90% identical to SEQ ID NO: 43, and the guide nucleic acid comprises a nucleotide sequence that is at least 90% identical to SEQ ID NO: 195. In some embodiments, the effector protein comprises an amino acid sequence that is at least 90% identical to SEQ ID NO: 44, and the guide nucleic acid comprises a nucleotide sequence that is at least 90% identical to SEQ ID NO: 196. In some embodiments, the effector protein comprises an amino acid sequence that is at least 90% identical to SEQ ID NO: 45, and the guide nucleic acid comprises a nucleotide sequence that is at least 90% identical to SEQ ID NO: 197. In some embodiments, the effector protein comprises an amino acid sequence that is at least 90% identical to SEQ ID NO: 46, and the guide nucleic acid comprises a nucleotide sequence that is at least 90% identical to SEQ ID NO: 198. In some embodiments, the effector protein comprises an amino acid sequence that is at least 90% identical to SEQ ID NO: 47, and the guide nucleic acid comprises a nucleotide sequence that is at least 90% identical to SEQ ID NO: 199. In some embodiments, the effector protein comprises an amino acid sequence that is at least 90% identical to SEQ ID NO: 47, and the guide nucleic acid comprises a nucleotide sequence that is at least 90% identical to SEQ ID NO: 200. In some embodiments, the effector protein comprises an amino acid sequence that is at least 90% identical to SEQ ID NO: 49, and the guide nucleic acid comprises a nucleotide sequence that is at least 90% identical to SEQ ID NO: 201. In some embodiments, the effector protein comprises an amino acid sequence that is at least 90% identical to SEQ ID NO: 51, and the guide nucleic acid comprises a nucleotide sequence that is at least 90% identical to SEQ ID NO: 202. In some embodiments, the effector protein comprises an amino acid sequence that is at least 90% identical to SEQ ID NO: 53, and the guide nucleic acid comprises a nucleotide sequence that is at least 90% identical to SEQ ID NO: 203. In some embodiments, the effector protein comprises an amino acid sequence that is at least 90% identical to SEQ ID NO: 53, and the guide nucleic acid comprises a nucleotide sequence that is at least 90% identical to SEQ ID NO: 204. In some embodiments, the effector protein comprises an amino acid sequence that is at least 90% identical to SEQ ID NO: 54, and the guide nucleic acid comprises a nucleotide sequence that is at least 90% identical to SEQ ID NO: 205. In some embodiments, the effector protein comprises an amino acid sequence that is at least 90% identical to SEQ ID NO: 55, and the guide nucleic acid comprises a nucleotide sequence that is at least 90% identical to SEQ ID NO: 206. In some embodiments, the effector protein comprises an amino acid sequence that is at least 90% identical to SEQ ID NO: 56, and the guide nucleic acid comprises a nucleotide sequence that is at least 90% identical to SEQ ID NO: 207. In some embodiments, the effector protein comprises an amino acid sequence that is at least 90% identical to SEQ ID NO: 57, and the guide nucleic acid comprises a nucleotide sequence that is at least 90% identical to SEQ ID NO: 208. In some embodiments, the effector protein comprises an amino acid sequence that is at least 90% identical to SEQ ID NO: 58, and the guide nucleic acid comprises a nucleotide sequence that is at least 90% identical to SEQ ID NO: 209. In some embodiments, the effector protein comprises an amino acid sequence that is at least 90% identical to SEQ ID NO: 59, and the guide nucleic acid comprises a nucleotide sequence that is at least 90% identical to SEQ ID NO: 210. In some embodiments, the effector protein comprises an amino acid sequence that is at least 90% identical to SEQ ID NO: 59, and the guide nucleic acid comprises a nucleotide sequence that is at least 90% identical to SEQ ID NO: 211. In some embodiments, the effector protein comprises an amino acid sequence that is at least 90% identical to SEQ ID NO: 60, and the guide nucleic acid comprises a nucleotide sequence that is at least 90% identical to SEQ ID NO: 212. In some embodiments, the effector protein comprises an amino acid sequence that is at least 90% identical to SEQ ID NO: 62, and the guide nucleic acid comprises a nucleotide sequence that is at least 90% identical to SEQ ID NO: 213. In some embodiments, the effector protein comprises an amino acid sequence that is at least 90% identical to SEQ ID NO: 63, and the guide nucleic acid comprises a nucleotide sequence that is at least 90% identical to SEQ ID NO: 214. In some embodiments, the effector protein comprises an amino acid sequence that is at least 90% identical to SEQ ID NO: 65, and the guide nucleic acid comprises a nucleotide sequence that is at least 90% identical to SEQ ID NO: 215. In some embodiments, the effector protein comprises an amino acid sequence that is at least 90% identical to SEQ ID NO: 66, and the guide nucleic acid comprises a nucleotide sequence that is at least 90% identical to SEQ ID NO: 216. In some embodiments, the effector protein comprises an amino acid sequence that is at least 90% identical to SEQ ID NO: 68, and the guide nucleic acid comprises a nucleotide sequence that is at least 90% identical to SEQ ID NO: 217. In some embodiments, the effector protein comprises an amino acid sequence that is at least 90% identical to SEQ ID NO: 71, and the guide nucleic acid comprises a nucleotide sequence that is at least 90% identical to SEQ ID NO: 218. In some embodiments, the effector protein comprises an amino acid sequence that is at least 90% identical to SEQ ID NO: 71, and the guide nucleic acid comprises a nucleotide sequence that is at least 90% identical to SEQ ID NO: 219. In some embodiments, the effector protein comprises an amino acid sequence that is at least 90% identical to SEQ ID NO: 73, and the guide nucleic acid comprises a nucleotide sequence that is at least 90% identical to SEQ ID NO: 220. In some embodiments, the effector protein comprises an amino acid sequence that is at least 90% identical to SEQ ID NO: 75, and the guide nucleic acid comprises a nucleotide sequence that is at least 90% identical to SEQ ID NO: 221. In some embodiments, the effector protein comprises an amino acid sequence that is at least 90% identical to SEQ ID NO: 75, and the guide nucleic acid comprises a nucleotide sequence that is at least 90% identical to SEQ ID NO: 222. In some embodiments, the effector protein comprises an amino acid sequence that is at least 90% identical to SEQ ID NO: 76, and the guide nucleic acid comprises a nucleotide sequence that is at least 90% identical to SEQ ID NO: 223. In some embodiments, the effector protein comprises an amino acid sequence that is at least 90% identical to SEQ ID NO: 6, and the guide nucleic acid comprises a nucleotide sequence that is at least 90% identical to SEQ ID NO: 224. In some embodiments, the effector protein comprises an amino acid sequence that is at least 90% identical to SEQ ID NO: 7, and the guide nucleic acid comprises a nucleotide sequence that is at least 90% identical to SEQ ID NO: 225. In some embodiments, the effector protein comprises an amino acid sequence that is at least 90% identical to SEQ ID NO: 25, and the guide nucleic acid comprises a nucleotide sequence that is at least 90% identical to SEQ ID NO: 226. In some embodiments, the effector protein comprises an amino acid sequence that is at least 90% identical to SEQ ID NO: 64, and the guide nucleic acid comprises a nucleotide sequence that is at least 90% identical to SEQ ID NO: 227.

In some embodiments, the guide nucleic acid comprises a nucleotide sequence that is at least 80%, at least 85%, at least 90%, at least 95%, at least 98% or 100% identical to a nucleotide sequence described herein (e.g., SEQ ID NOS: 228-425). In some embodiments, the guide nucleic acid comprises at least 8, at least 9, at least 10, at least 11, at least 12, at least 13, at least 14, at least 15, at least 16, at least 17, at least 18, at least 19, or at least 20 contiguous nucleotides of a nucleotide sequence described herein (e.g., SEQ ID NOS: 228-425). In some embodiments, the guide nucleic acid comprises at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, or at least 220 contiguous nucleotides of a nucleotide sequence described herein (e.g., SEQ ID NOS: 228-425).

In some embodiments, the effector protein comprises a specific amino acid sequence as described herein (e.g., TABLE 1) with a specific guide nucleic acid as described herein (e.g., TABLE 6). In some embodiments, the effector protein comprises an amino acid sequence that is at least 90% identical to SEQ ID NO: 1, and the guide nucleic acid comprises a nucleotide sequence that is at least 90% identical to SEQ ID NO: 228 or 327. In some embodiments, the effector protein comprises an amino acid sequence that is at least 90% identical to SEQ ID NO: 2, and the guide nucleic acid comprises a nucleotide sequence that is at least 90% identical to SEQ ID NO: 229 or 328. In some embodiments, the effector protein comprises an amino acid sequence that is at least 90% identical to SEQ ID NO: 3, and the guide nucleic acid comprises a nucleotide sequence that is at least 90% identical to SEQ ID NO: 230 or 231 or SEQ ID NO: 329 or 330. In some embodiments, the effector protein comprises an amino acid sequence that is at least 90% identical to SEQ ID NO: 4, and the guide nucleic acid comprises a nucleotide sequence that is at least 90% identical to SEQ ID NO: 232 or 331. In some embodiments, the effector protein comprises an amino acid sequence that is at least 90% identical to SEQ ID NO: 5, and the guide nucleic acid comprises a nucleotide sequence that is at least 90% identical to SEQ ID NO: 233 or 332. In some embodiments, the effector protein comprises an amino acid sequence that is at least 90% identical to SEQ ID NO: 6, and the guide nucleic acid comprises a nucleotide sequence that is at least 90% identical to SEQ ID NO: 234 or 333. In some embodiments, the effector protein comprises an amino acid sequence that is at least 90% identical to SEQ ID NO: 7, and the guide nucleic acid comprises a nucleotide sequence that is at least 90% identical to SEQ ID NO: 235 or 334. In some embodiments, the effector protein comprises an amino acid sequence that is at least 90% identical to SEQ ID NO: 8, and the guide nucleic acid comprises a nucleotide sequence that is at least 90% identical to SEQ ID NO: 236 or 335. In some embodiments, the effector protein comprises an amino acid sequence that is at least 90% identical to SEQ ID NO: 9, and the guide nucleic acid comprises a nucleotide sequence that is at least 90% identical to SEQ ID NO: 237 or 336. In some embodiments, the effector protein comprises an amino acid sequence that is at least 90% identical to SEQ ID NO: 10, and the guide nucleic acid comprises a nucleotide sequence that is at least 90% identical to SEQ ID NO: 238 or 337. In some embodiments, the effector protein comprises an amino acid sequence that is at least 90% identical to SEQ ID NO: 11, and the guide nucleic acid comprises a nucleotide sequence that is at least 90% identical to SEQ ID NO: 239 or 240 or SEQ ID NO: 338 or 339. In some embodiments, the effector protein comprises an amino acid sequence that is at least 90% identical to SEQ ID NO: 12, and the guide nucleic acid comprises a nucleotide sequence that is at least 90% identical to SEQ ID NO: 241 or 340. In some embodiments, the effector protein comprises an amino acid sequence that is at least 90% identical to SEQ ID NO: 13, and the guide nucleic acid comprises a nucleotide sequence that is at least 90% identical to SEQ ID NO: 242 or 341. In some embodiments, the effector protein comprises an amino acid sequence that is at least 90% identical to SEQ ID NO: 14, and the guide nucleic acid comprises a nucleotide sequence that is at least 90% identical to SEQ ID NO: 243 or 342. In some embodiments, the effector protein comprises an amino acid sequence that is at least 90% identical to SEQ ID NO: 15, and the guide nucleic acid comprises a nucleotide sequence that is at least 90% identical to SEQ ID NO: 244 or 343. In some embodiments, the effector protein comprises an amino acid sequence that is at least 90% identical to SEQ ID NO: 16, and the guide nucleic acid comprises a nucleotide sequence that is at least 90% identical to SEQ ID NO: 245 or 246 or SEQ ID NO: 344 or 345. In some embodiments, the effector protein comprises an amino acid sequence that is at least 90% identical to SEQ ID NO: 17, and the guide nucleic acid comprises a nucleotide sequence that is at least 90% identical to SEQ ID NO: 247 or 248 or SEQ ID NO: 346 or 347. In some embodiments, the effector protein comprises an amino acid sequence that is at least 90% identical to SEQ ID NO: 18, and the guide nucleic acid comprises a nucleotide sequence that is at least 90% identical to SEQ ID NO: 249 or 348. In some embodiments, the effector protein comprises an amino acid sequence that is at least 90% identical to SEQ ID NO: 19, and the guide nucleic acid comprises a nucleotide sequence that is at least 90% identical to SEQ ID NO: 250 or 251 or SEQ ID NO: 349 or 350. In some embodiments, the effector protein comprises an amino acid sequence that is at least 90% identical to SEQ ID NO: 20, and the guide nucleic acid comprises a nucleotide sequence that is at least 90% identical to SEQ ID NO: 252 or 253 or SEQ ID NO: 351 or 352. In some embodiments, the effector protein comprises an amino acid sequence that is at least 90% identical to SEQ ID NO: 21, and the guide nucleic acid comprises a nucleotide sequence that is at least 90% identical to SEQ ID NO: 254 or 353. In some embodiments, the effector protein comprises an amino acid sequence that is at least 90% identical to SEQ ID NO: 22, and the guide nucleic acid comprises a nucleotide sequence that is at least 90% identical to SEQ ID NO: 255 or 354. In some embodiments, the effector protein comprises an amino acid sequence that is at least 90% identical to SEQ ID NO: 23, and the guide nucleic acid comprises a nucleotide sequence that is at least 90% identical to SEQ ID NO: 256 or 355. In some embodiments, the effector protein comprises an amino acid sequence that is at least 90% identical to SEQ ID NO: 24, and the guide nucleic acid comprises a nucleotide sequence that is at least 90% identical to SEQ ID NO: 257 or 356. In some embodiments, the effector protein comprises an amino acid sequence that is at least 90% identical to SEQ ID NO: 25, and the guide nucleic acid comprises a nucleotide sequence that is at least 90% identical to SEQ ID NO: 258 or 259 or SEQ ID NO: 357 or 358. In some embodiments, the effector protein comprises an amino acid sequence that is at least 90% identical to SEQ ID NO: 26, and the guide nucleic acid comprises a nucleotide sequence that is at least 90% identical to SEQ ID NO: 260 or 261 or SEQ ID NO: 359 or 360. In some embodiments, the effector protein comprises an amino acid sequence that is at least 90% identical to SEQ ID NO: 27, and the guide nucleic acid comprises a nucleotide sequence that is at least 90% identical to SEQ ID NO: 262 or 361. In some embodiments, the effector protein comprises an amino acid sequence that is at least 90% identical to SEQ ID NO: 28, and the guide nucleic acid comprises a nucleotide sequence that is at least 90% identical to SEQ ID NO: 263 or 264 or SEQ ID NO: 362 or 363. In some embodiments, the effector protein comprises an amino acid sequence that is at least 90% identical to SEQ ID NO: 29, and the guide nucleic acid comprises a nucleotide sequence that is at least 90% identical to SEQ ID NO: 265 or 364. In some embodiments, the effector protein comprises an amino acid sequence that is at least 90% identical to SEQ ID NO: 30, and the guide nucleic acid comprises a nucleotide sequence that is at least 90% identical to SEQ ID NO: 266 or 365. In some embodiments, the effector protein comprises an amino acid sequence that is at least 90% identical to SEQ ID NO: 31, and the guide nucleic acid comprises a nucleotide sequence that is at least 90% identical to SEQ ID NO: 267 or 366. In some embodiments, the effector protein comprises an amino acid sequence that is at least 90% identical to SEQ ID NO: 32, and the guide nucleic acid comprises a nucleotide sequence that is at least 90% identical to SEQ ID NO: 268 or 367. In some embodiments, the effector protein comprises an amino acid sequence that is at least 90% identical to SEQ ID NO: 33, and the guide nucleic acid comprises a nucleotide sequence that is at least 90% identical to SEQ ID NO: 269 or 368. In some embodiments, the effector protein comprises an amino acid sequence that is at least 90% identical to SEQ ID NO: 34, and the guide nucleic acid comprises a nucleotide sequence that is at least 90% identical to SEQ ID NO: 270 or 369. In some embodiments, the effector protein comprises an amino acid sequence that is at least 90% identical to SEQ ID NO: 35, and the guide nucleic acid comprises a nucleotide sequence that is at least 90% identical to SEQ ID NO: 271 or 370. In some embodiments, the effector protein comprises an amino acid sequence that is at least 90% identical to SEQ ID NO: 36, and the guide nucleic acid comprises a nucleotide sequence that is at least 90% identical to SEQ ID NO: 272 or 371. In some embodiments, the effector protein comprises an amino acid sequence that is at least 90% identical to SEQ ID NO: 37, and the guide nucleic acid comprises a nucleotide sequence that is at least 90% identical to SEQ ID NO: 273 or 372. In some embodiments, the effector protein comprises an amino acid sequence that is at least 90% identical to SEQ ID NO: 38, and the guide nucleic acid comprises a nucleotide sequence that is at least 90% identical to SEQ ID NO: 274 or 275 or SEQ ID NO: 373 or 374. In some embodiments, the effector protein comprises an amino acid sequence that is at least 90% identical to SEQ ID NO: 39, and the guide nucleic acid comprises a nucleotide sequence that is at least 90% identical to SEQ ID NO: 276 or 375. In some embodiments, the effector protein comprises an amino acid sequence that is at least 90% identical to SEQ ID NO: 40, and the guide nucleic acid comprises a nucleotide sequence that is at least 90% identical to SEQ ID NO: 277 or 278 or SEQ ID NO: 376 or 377. In some embodiments, the effector protein comprises an amino acid sequence that is at least 90% identical to SEQ ID NO: 41, and the guide nucleic acid comprises a nucleotide sequence that is at least 90% identical to SEQ ID NO: 279 or 378. In some embodiments, the effector protein comprises an amino acid sequence that is at least 90% identical to SEQ ID NO: 42, and the guide nucleic acid comprises a nucleotide sequence that is at least 90% identical to SEQ ID NO: 280 or 281 or SEQ ID NO: 379 or 380. In some embodiments, the effector protein comprises an amino acid sequence that is at least 90% identical to SEQ ID NO: 43, and the guide nucleic acid comprises a nucleotide sequence that is at least 90% identical to SEQ ID NO: 282 or 381. In some embodiments, the effector protein comprises an amino acid sequence that is at least 90% identical to SEQ ID NO: 44, and the guide nucleic acid comprises a nucleotide sequence that is at least 90% identical to SEQ ID NO: 283 or 382. In some embodiments, the effector protein comprises an amino acid sequence that is at least 90% identical to SEQ ID NO: 45, and the guide nucleic acid comprises a nucleotide sequence that is at least 90% identical to SEQ ID NO: 284 or 384. In some embodiments, the effector protein comprises an amino acid sequence that is at least 90% identical to SEQ ID NO: 46, and the guide nucleic acid comprises a nucleotide sequence that is at least 90% identical to SEQ ID NO: 285 or 384. In some embodiments, the effector protein comprises an amino acid sequence that is at least 90% identical to SEQ ID NO: 47, and the guide nucleic acid comprises a nucleotide sequence that is at least 90% identical to SEQ ID NO: 286 or 287 or SEQ ID NO: 386 or 387. In some embodiments, the effector protein comprises an amino acid sequence that is at least 90% identical to SEQ ID NO: 48, and the guide nucleic acid comprises a nucleotide sequence that is at least 90% identical to SEQ ID NO: 288 or 387. In some embodiments, the effector protein comprises an amino acid sequence that is at least 90% identical to SEQ ID NO: 49, and the guide nucleic acid comprises a nucleotide sequence that is at least 90% identical to SEQ ID NO: 289 or 290 or SEQ ID NO: 388 or 389. In some embodiments, the effector protein comprises an amino acid sequence that is at least 90% identical to SEQ ID NO: 50, and the guide nucleic acid comprises a nucleotide sequence that is at least 90% identical to SEQ ID NO: 291 or 292 or SEQ ID NO 390 or 391. In some embodiments, the effector protein comprises an amino acid sequence that is at least 90% identical to SEQ ID NO: 51, and the guide nucleic acid comprises a nucleotide sequence that is at least 90% identical to SEQ ID NO: 293 or 392. In some embodiments, the effector protein comprises an amino acid sequence that is at least 90% identical to SEQ ID NO: 52, and the guide nucleic acid comprises a nucleotide sequence that is at least 90% identical to SEQ ID NO: 294 or 393. In some embodiments, the effector protein comprises an amino acid sequence that is at least 90% identical to SEQ ID NO: 53, and the guide nucleic acid comprises a nucleotide sequence that is at least 90% identical to SEQ ID NO: 295 or 296 or SEQ ID NO: 394 or 395. In some embodiments, the effector protein comprises an amino acid sequence that is at least 90% identical to SEQ ID NO: 54, and the guide nucleic acid comprises a nucleotide sequence that is at least 90% identical to SEQ ID NO: 297 or 395. In some embodiments, the effector protein comprises an amino acid sequence that is at least 90% identical to SEQ ID NO: 55, and the guide nucleic acid comprises a nucleotide sequence that is at least 90% identical to SEQ ID NO: 298 or 397. In some embodiments, the effector protein comprises an amino acid sequence that is at least 90% identical to SEQ ID NO: 56, and the guide nucleic acid comprises a nucleotide sequence that is at least 90% identical to SEQ ID NO: 299 or 398. In some embodiments, the effector protein comprises an amino acid sequence that is at least 90% identical to SEQ ID NO: 57, and the guide nucleic acid comprises a nucleotide sequence that is at least 90% identical to SEQ ID NO: 300 or 399. In some embodiments, the effector protein comprises an amino acid sequence that is at least 90% identical to SEQ ID NO: 58, and the guide nucleic acid comprises a nucleotide sequence that is at least 90% identical to SEQ ID NO: 301 or 302 or SEQ ID NO: 400 or 401. In some embodiments, the effector protein comprises an amino acid sequence that is at least 90% identical to SEQ ID NO: 59, and the guide nucleic acid comprises a nucleotide sequence that is at least 90% identical to SEQ ID NO: 303 or 304 or SEQ ID NO: 402 or 403. In some embodiments, the effector protein comprises an amino acid sequence that is at least 90% identical to SEQ ID NO: 60, and the guide nucleic acid comprises a nucleotide sequence that is at least 90% identical to SEQ ID NO: 305 or 404. In some embodiments, the effector protein comprises an amino acid sequence that is at least 90% identical to SEQ ID NO: 61, and the guide nucleic acid comprises a nucleotide sequence that is at least 90% identical to SEQ ID NO: 306 or 405. In some embodiments, the effector protein comprises an amino acid sequence that is at least 90% identical to SEQ ID NO: 62, and the guide nucleic acid comprises a nucleotide sequence that is at least 90% identical to SEQ ID NO: 307 or 406. In some embodiments, the effector protein comprises an amino acid sequence that is at least 90% identical to SEQ ID NO: 63, and the guide nucleic acid comprises a nucleotide sequence that is at least 90% identical to SEQ ID NO: 308 or 407. In some embodiments, the effector protein comprises an amino acid sequence that is at least 90% identical to SEQ ID NO: 64, and the guide nucleic acid comprises a nucleotide sequence that is at least 90% identical to SEQ ID NO: 309 or 408. In some embodiments, the effector protein comprises an amino acid sequence that is at least 90% identical to SEQ ID NO: 65, and the guide nucleic acid comprises a nucleotide sequence that is at least 90% identical to SEQ ID NO: 310 or 409. In some embodiments, the effector protein comprises an amino acid sequence that is at least 90% identical to SEQ ID NO: 66, and the guide nucleic acid comprises a nucleotide sequence that is at least 90% identical to SEQ ID NO: 311 or 312 or SEQ ID NO: 410 or 411. In some embodiments, the effector protein comprises an amino acid sequence that is at least 90% identical to SEQ ID NO: 67, and the guide nucleic acid comprises a nucleotide sequence that is at least 90% identical to SEQ ID NO: 313 or 314 or SEQ ID NO: 412 or 413. In some embodiments, the effector protein comprises an amino acid sequence that is at least 90% identical to SEQ ID NO: 68, and the guide nucleic acid comprises a nucleotide sequence that is at least 90% identical to SEQ ID NO: 315 or 414. In some embodiments, the effector protein comprises an amino acid sequence that is at least 90% identical to SEQ ID NO: 69, and the guide nucleic acid comprises a nucleotide sequence that is at least 90% identical to SEQ ID NO: 316 or 415. In some embodiments, the effector protein comprises an amino acid sequence that is at least 90% identical to SEQ ID NO: 70, and the guide nucleic acid comprises a nucleotide sequence that is at least 90% identical to SEQ ID NO: 317 or 416. In some embodiments, the effector protein comprises an amino acid sequence that is at least 90% identical to SEQ ID NO: 71, and the guide nucleic acid comprises a nucleotide sequence that is at least 90% identical to SEQ ID NO: 318 or 319 or SEQ ID NO: 417 or 418. In some embodiments, the effector protein comprises an amino acid sequence that is at least 90% identical to SEQ ID NO: 72, and the guide nucleic acid comprises a nucleotide sequence that is at least 90% identical to SEQ ID NO: 320 or 419. In some embodiments, the effector protein comprises an amino acid sequence that is at least 90% identical to SEQ ID NO: 73, and the guide nucleic acid comprises a nucleotide sequence that is at least 90% identical to SEQ ID NO: 321 or 420. In some embodiments, the effector protein comprises an amino acid sequence that is at least 90% identical to SEQ ID NO: 74, and the guide nucleic acid comprises a nucleotide sequence that is at least 90% identical to SEQ ID NO: 322 or 421. In some embodiments, the effector protein comprises an amino acid sequence that is at least 90% identical to SEQ ID NO: 75, and the guide nucleic acid comprises a nucleotide sequence that is at least 90% identical to SEQ ID NO: 323 or 324 or SEQ ID NO: 422 or 423. In some embodiments, the effector protein comprises an amino acid sequence that is at least 90% identical to SEQ ID NO: 76, and the guide nucleic acid comprises a nucleotide sequence that is at least 90% identical to SEQ ID NO: 325 or 424. In some embodiments, the effector protein comprises an amino acid sequence that is at least 90% identical to SEQ ID NO: 77, and the guide nucleic acid comprises a nucleotide sequence that is at least 90% identical to SEQ ID NO: 326 or 425. In some embodiments, the effector protein comprises an amino acid sequence that is at least 90% identical to SEQ ID NO: 78.

In some embodiments, the effector protein comprises a nuclear localization signal.

In some embodiments, the length of the effector protein is at least 300, at least 350, at least 400, at least 450, at least 500, at least 550, at least 600, at least 650, at least 700, at least 750, at least 800, at least 850, at least 900, at least 950, at least 1000, at least 1050, at least 1100, at least 1150, at least 1200, at least 1250, or at least 1300 linked amino acid residues. In some embodiments, the length of the effector protein is less than about 1900 linked amino acids. In some embodiments, the length of the effector protein is about 300 to about 400, about 350 to about 450, about 400 to about 500, about 450 to about 550, about 500 to about 600, about 550 to about 650, about 600 to about 700, about 650 to about 750, about 700 to about 800, about 750 to about 850, about 800 to about 900, about 850 to about 950, about 900 to about 1000, about 950 to about 1050, about 1000 to about 1100, about 1050 to about 1150, about 1100 to about 1200, about 1150 to about 1250, about 1200 to about 1300, about 1250 to about 1350, about 1300 to about 1400, about 1350 to about 1450, about 1400 to about 1500, about 1450 to about 1550, about 1500 to about 1600, about 1550 to about 1650, about 1600 to about 1700, about 1650 to about 1750, about 1700 to about 1800, about 1750 to about 1850, or about 1800 to about 1900 linked amino acids.

In some embodiments, compositions described herein comprise a donor nucleic acid.

In some embodiments, compositions described herein comprise a fusion partner protein linked to the effector protein. In some embodiments, the fusion partner protein is directly fused to the N terminus or C terminus of the effector protein via an amide bond. In some embodiments, the fusion partner protein is directly fused to the N terminus or C terminus of the effector protein via a peptide linker. In some embodiments, the fusion partner protein comprises a polypeptide selected from a deaminase, a transcriptional activator, a transcriptional repressor, or a functional domain thereof. In some embodiments, the effector protein comprises at least one mutation that reduces its nuclease activity relative to the effector protein without the mutation as measured in a cleavage assay, optionally wherein the effector protein is a catalytically inactive nuclease.

In some aspects, provided herein are compositions comprising a nucleic acid expression vector, wherein the nucleic acid vector encodes at least one of the effector protein and the guide nucleic acid of a composition described herein. In some embodiments, a donor nucleic acid, optionally wherein the donor nucleic acid is encoded by the nucleic acid expression vector or an additional nucleic acid expression vector. In some embodiments, the nucleic acid expression vector is a viral vector. In some embodiments, the viral vector is an adeno-associated viral (AAV) vector.

In some aspects, provided herein are compositions comprising a virus, wherein the virus comprises a composition as described herein.

In some aspects, provided herein are pharmaceutical compositions comprising a composition as described herein, and a pharmaceutically acceptable excipient.

In some aspects, provided herein are systems comprising a composition as described herein, and at least one detection reagent for detecting a target nucleic acid. In some embodiments, the at least one detection reagent is selected from a reporter nucleic acid, a detection moiety, an additional effector protein, or a combination thereof, optionally wherein the reporter nucleic acid comprises a fluorophore, a quencher, or a combination thereof. In some embodiments, the system comprises at least one amplification reagent for amplifying a target nucleic acid. In some embodiments, the at least one amplification reagent is selected from the group consisting of a primer, a polymerase, a deoxynucleoside triphosphate (dNTP), a ribonucleoside triphosphate (rNTP), and combinations thereof. In some embodiments, the system comprises a device with a chamber or solid support for containing the composition, target nucleic acid, detection reagent or combination thereof.

In some aspects, provided herein are methods of detecting a target nucleic acid in a sample, comprising the steps of: (a) contacting the sample with: (i) a composition as described herein or a system as described herein; and (ii) a reporter nucleic acid comprising a detectable moiety that produces a detectable signal in the presence of the target nucleic acid and the composition or system, and (b) detecting the detectable signal. In some embodiments, the reporter nucleic acid comprises a fluorophore, a quencher, or a combination thereof, and wherein the detecting comprises detecting a fluorescent signal. In some embodiments, the method comprises reverse transcribing the target nucleic acid, amplifying the target nucleic acid, in vitro transcribing the target nucleic acid, or any combination thereof. In some embodiments, the method comprises reverse transcribing the target nucleic acid and/or amplifying the target nucleic acid before contacting the sample with the composition. In some embodiments, the method comprises reverse transcribing the target nucleic acid and/or amplifying the target nucleic acid after contacting the sample with the composition. In some embodiments, the amplifying comprises isothermal amplification. In some embodiments, the target nucleic acid is from a pathogen. In some embodiments, the pathogen is a virus. In some embodiments, the target nucleic acid comprises RNA. In some embodiments, the target nucleic acid comprises DNA.

In some aspects, provided herein are methods of modifying a target nucleic acid, the methods comprising contacting the target nucleic acid with a composition as described herein, or a system as described herein, thereby modifying the target nucleic acid. In some embodiments, modifying the target nucleic acid comprises cleaving the target nucleic acid, deleting a nucleotide of the target nucleic acid, inserting a nucleotide into the target nucleic acid, substituting a nucleotide of the target nucleic acid with an alternative nucleotide or an additional nucleotide, or any combination thereof. In some embodiments, the method comprises contacting the target nucleic acid with a donor nucleic acid. In some embodiments, the target nucleic acid comprises a mutation associated with a disease. In some embodiments, the disease is selected from an autoimmune disease, a cancer, an inherited disorder, an ophthalmological disorder, a metabolic disorder, or a combination thereof. In some embodiments, the disease is cystic fibrosis, thalassemia, Duchenne muscular dystrophy, myotonic dystrophy Type 1, or sickle cell anemia. In some embodiments, contacting the target nucleic acid comprises contacting a cell, wherein the target nucleic acid is located in the cell. In some embodiments, the contacting occurs in vitro. In some embodiments, the contacting occurs in vivo. In some embodiments, the contacting occurs ex vivo.

In some aspects, provided herein are cells comprising a composition as described herein.

In some aspects, provided herein are cells modified by a composition as described herein.

In some aspects, provided herein are cells modified by a system as described herein.

In some aspects, provided herein are cells comprising a modified target nucleic acid, wherein the modified target nucleic acid is a target nucleic acid modified according to a method as described herein. In some embodiments, the cell is a eukaryotic cell. In some embodiments, the cell is a mammalian cell. In some embodiments, the cell is a prokaryotic cell. In some embodiments, the cell is a plant cell. In some embodiments, the cell is an animal cell. In some embodiments, the cell is a T cell, optionally wherein the T cell is a natural killer T cell (NKT). In some embodiments, the cell is a chimeric antigen receptor T cell (CAR T-cell). In some embodiments, the cell is an induced pluripotent stem cell (iPSC).

In some aspects, provided herein are populations of cells as described herein.

In some aspects, provided herein are methods of producing a protein, the methods comprising, (i) contacting a cell comprising a target nucleic acid to a composition as described herein, thereby editing the target nucleic acid to produce a modified cell comprising a modified target nucleic acid; and (ii) producing a protein from the cell that is encoded, transcriptionally affected, or translationally affected by the modified nucleic acid.

In some aspects, provided herein are methods of treating a disease comprising administering to a subject in need thereof a composition as described herein, or a cell as described herein.

In some aspects, provided herein are systems for modifying a target nucleic acid. In some embodiments, the system comprises at least two components each individually comprising one of the following: an effector protein or a nucleic acid encoding the effector protein; and a guide nucleic acid or a nucleic acid encoding the guide nucleic acid. In some embodiments, the effector protein comprises an amino acid sequence that is at least 75%, at least 80%, at least 85%, at least 90%, at least 95%, at least 98%, at least 99%, or 100% identical to any one of SEQ ID NOS: 1-78 and 499. In some embodiments, the effector protein comprises about 100, about 120, about 140, about 160, about 180, about 200, about 220, about 240, about 260, about 280, about 300, about 320, about 340, about 360, about 380, about 400, about 420, about 440, about 460, about 480, about 500, about 520, about 540, about 560, about 580, about 600, about 620, about 640, about 660, about 680, about 700, about 720, about 740, about 760, about 780, about 800, about 820, about 840, about 860, about 880, about 900, about 920, about 940, about 960, about 980, about 1000, about 1020, about 1040, about 1060, about 1080, about 1100, about 1120, about 1140, about 1160, about 1180, about 1200, about 1220, about 1240, about 1260, about 1280, about 1300, about 1320, about 1340, about 1360, about 1380, or about 1400 contiguous amino acids of a sequence selected from SEQ ID NOS: 1-78 and 499. In some embodiments, at least a portion of the guide nucleic acid is complementary to a target sequence of a target nucleic acid. In some embodiments, a length of the effector protein is at least 300, at least 350, at least 400, at least 450, at least 500, at least 550, at least 600, at least 650, at least 700, at least 750, at least 800, at least 850, at least 900, at least 950, at least 1000, at least 1050, at least 1100, at least 1150, at least 1200, at least 1250, or at least 1300 linked amino acid residues. In some embodiments, at least one of the effector proteins, the guide nucleic acid, and the combination thereof, are not naturally occurring. In some embodiments, the target nucleic acid comprises a protospacer adjacent motif (PAM). In some embodiments, the PAM comprises a nucleotide sequence that is at least 80%, at least 85%, at least 90%, at least 95%, at least 98% or 100% identical to a nucleotide sequence selected from SEQ ID NOS: 79-122. In some embodiments, the guide nucleic acid comprises a repeat sequence that comprises at least 8, at least 9, at least 10, at least 11, at least 12, at least 13, at least 14, at least 15, at least 16, at least 17, at least 18, at least 19, or at least 20 contiguous nucleotides of a nucleotide sequence selected from SEQ ID NOS: 123-165. In some embodiments, the guide nucleic acid comprises at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, or at least 220 contiguous nucleotides of a nucleotide sequence selected from SEQ ID NOS: 166-227, 327-425 and 436-498. In some embodiments, the effector protein comprises a nuclear localization signal comprising any one of the amino acid sequences recited in TABLE 1.1. In some embodiments, the system further comprises a component comprising a donor nucleic acid. In some embodiments, the system further comprises a component comprising a fusion partner protein. In some embodiments, the fusion partner protein is fused to the effector protein. In some embodiments, the fusion partner protein is not fused to the effector protein. In some embodiments, the effector protein comprises a catalytic activity that is 100%, 90%, 80%, 70%, 60%, 50%, 40%, 30%, 20%, 10%, 5%, 1%, or less, relative to a naturally occurring counterpart.

### INCORPORATION BY REFERENCE

All publications, patents, and patent applications mentioned in this specification are herein incorporated by reference in their entirety for any purpose and to the same extent as if each individual publication, patent, or patent application was specifically and individually indicated to be incorporated by reference.

### BRIEF DESCRIPTION OF THE DRAWINGS

FIG. 1 shows an exemplary gel that demonstrates effector protein-sgRNA complexes that were capable of cis-cleavage.

FIGS. 2A-2D illustrate PAM preferences for different effector proteins disclosed herein. Frequency of nucleotides at each PAM position was independently calculated using a position frequency matrix (PFM) and plotted as a WebLogo. The effector protein, corresponding guide nucleic acid, and consensus PAM sequence are indicated in TABLE 9.

FIG. 3 is a graphical depiction of an experiment employing a split protein design and split RNA design according to an embodiment of the disclosure.

FIG. 4A summarizes results of % precise gene editing generated by a nD2L-932 and reverse transcriptase in HEK293T cells. The following conditions were assayed (results depicted from left to right): negative control (Neg Ctrl) and four guide nucleic acids (R15PI1, R15P15, R20P11, and R20P15).

FIG. 4B summarizes results of % indel generated by a nD2L-932 and reverse transcriptase in HEK293T cells. The following conditions were assayed (results depicted from left to right): negative control (Neg Ctrl) and four guide nucleic acids (R15PI1, R15P15, R20P11, and R20P15).

### DETAILED DESCRIPTION OF THE INVENTION

It is to be understood that both the foregoing general description and the following detailed description are exemplary, and explanatory only, and are not restrictive of the disclosure.

The section headings used herein are for organizational purposes only and are not to be construed as limiting the subject matter described.

### Definitions

Unless otherwise indicated, all technical terms used herein have the same meaning as commonly understood by one of ordinary skill in the art to which this invention belongs. Unless otherwise indicated or obvious from context, the following terms have the following meanings:

As used in this specification and the appended claims, the singular forms “a,” “an,” and “the” include plural references unless the context clearly dictates otherwise.

Any reference to “or” herein is intended to encompass “and/or” unless otherwise stated. As used herein, the term “and/or” includes any and all combinations of one or more of the associated listed items.

Use of the term “including” as well as other forms, such as “includes” and “included,” is not limiting.

As used herein, the term, “comprise” and its grammatical equivalents, specifies the presence of stated features, integers, steps, operations, elements, and/or components, but does not preclude the presence or addition of one or more other features, integers, steps, operations, elements, components, and/or groups thereof.

As used herein, the term, “about,” in reference to a number or range of numbers, is understood to mean the stated number and numbers +/−10% thereof, or 10% below the lower listed limit and 10% above the higher listed limit for the values listed for a range.

The terms, “percent identity,” “% identity,” “% identical” and grammatical equivalents thereof, as used herein, refer to the extent to which two sequences (nucleotide or amino acid) have the same residue at the same positions in an alignment. For example, “an amino acid sequence is X % identical to SEQ ID NO: Y” can refer to % identity of the amino acid sequence to SEQ ID NO: Y and is elaborated as X % of residues in the amino acid sequence are identical to the residues of sequence disclosed in SEQ ID NO: Y. Generally, computer programs can be employed for such calculations. Illustrative programs that compare and align pairs of sequences, include ALIGN (Myers and Miller, Comput Appl Biosci. 1988 March; 4(1):11-7), FASTA (Pearson and Lipman, Proc Natl Acad Sci USA. 1988 April; 85(8):2444-8; Pearson, Methods Enzymol. 1990; 183:63-98) and gapped BLAST (Altschul et al., Nucleic Acids Res. 1997 Sep. 1; 25(17):3389-40), BLASTP, BLASTN, or GCG (Devereux et al., Nucleic Acids Res. 1984 Jan. 11; 12(1 Pt 1):387-95).

The terms, “amplification,” “amplifying” and grammatical equivalents thereof, as used herein, refers to a process by which a nucleic acid molecule is enzymatically copied to generate a plurality of nucleic acid molecules containing the same sequence as the original nucleic acid molecule or a distinguishable portion thereof.

The term, “base editing enzyme,” as used herein, refers to a protein, polypeptide, or fragment thereof that is capable of catalyzing the chemical modification of a nucleobase of a deoxyribonucleotide or a ribonucleotide. Such a base editing enzyme, for example, is capable of catalyzing a reaction that modifies a nucleobase that is present in a nucleic acid molecule, such as DNA or RNA (single stranded or double stranded). Non-limiting examples of the type of modification that a base editing enzyme is capable of catalyzing includes converting an existing nucleobase to a different nucleobase, such as converting a cytosine to a guanine or thymine or converting an adenine to a guanine, hydrolytic deamination of an adenine or adenosine, or methylation of cytosine (e.g., CpG, CpA, CpT or CpC). A base editing enzyme itself may or may not bind to the nucleic acid molecule containing the nucleobase.

The term, “base editor,” as used herein refers to a fusion protein comprising a base editing enzyme fused to an effector protein. The base editor is functional when the effector protein is coupled to a guide nucleic acid. The guide nucleic acid imparts sequence specific activity to the base editor. By way of non-limiting example, the effector protein may comprise a catalytically inactive effector protein. Also, by way of non-limiting example, the base editing enzyme may comprise deaminase activity. Additional base editors are described herein.

The term, “catalytically inactive effector protein,” as used herein, refers to an effector protein that is modified relative to a naturally-occurring effector protein to have a reduced or eliminated catalytic activity relative to that of the naturally-occurring effector protein, but retains its ability to interact with a guide nucleic acid. The catalytic activity that is reduced or eliminated is often a nuclease activity. The naturally-occurring effector protein may be a wildtype protein. In some embodiments, the catalytically inactive effector protein is referred to as a catalytically inactive variant of an effector protein, e.g., a Cas effector protein.

The term, “cis cleavage,” as used herein, refers to cleavage (hydrolysis of a phosphodiester bond) of a target nucleic acid by an effector protein complexed with a guide nucleic acid refers to cleavage of a target nucleic acid that is hybridized to a guide nucleic acid, wherein cleavage occurs within or directly adjacent to the region of the target nucleic acid that is hybridized to the guide nucleic acid.

The terms, “complementary” and complementarity,” as used herein with reference to a nucleic acid molecule or nucleotide sequence, refer to the characteristic of a polynucleotide having nucleotides that base pair with their Watson-Crick counterparts (C with G; or A with T) in a reference nucleic acid. For example, when every nucleotide in a polynucleotide forms a base pair with a reference nucleic acid, that polynucleotide is said to be 100% complementary to the reference nucleic acid. In a double stranded DNA or RNA sequence, the upper (sense) strand sequence is in general, understood as going in the direction from its 5′- to 3′-end, and the complementary sequence is thus understood as the sequence of the lower (antisense) strand in the same direction as the upper strand. Following the same logic, the reverse sequence is understood as the sequence of the upper strand in the direction from its 3′- to its 5′-end, while the ‘reverse complement’ sequence or the ‘reverse complementary’ sequence is understood as the sequence of the lower strand in the direction of its 5′- to its 3′-end. Each nucleotide in a double stranded DNA or RNA molecule that is paired with its Watson-Crick counterpart called its complementary nucleotide.

The term, “cleavage assay,” as used herein, refers to an assay designed to visualize, quantitate, or identify cleavage of a nucleic acid. In some cases, the cleavage activity may be cis-cleavage activity. In some cases, the cleavage activity may be trans-cleavage activity.

The term, “clustered regularly interspaced short palindromic repeats (CRISPR),” as used herein, refers to a segment of DNA found in the genomes of certain prokaryotic organisms, including some bacteria and archaea, that includes repeated short sequences of nucleotides interspersed at regular intervals between unique sequences of nucleotides derived from the DNA of a pathogen (e.g., virus) that had previously infected the organism and that functions to protect the organism against future infections by the same pathogen.

The terms, “CRISPR RNA” and “crRNA,” as used herein, refer to a type of guide nucleic acid, wherein the nucleic acid is RNA, comprising a first sequence, often referred to herein as a “spacer sequence,” that hybridizes to a target sequence of a target nucleic acid, and a second sequence that either a) hybridizes to a portion of a tracrRNA or b) is capable of being non-covalently bound by an effector protein. In some embodiments, the crRNA is covalently linked to an additional nucleic acid (e.g., a tracrRNA) that interacts with the effector protein.

The term, “detectable signal,” as used herein, refers to a signal that can be detected using optical, fluorescent, chemiluminescent, electrochemical, and other detection methods known in the art.

The term, “donor nucleic acid,” as used herein, refers to nucleic acid that is incorporated into a target nucleic acid.

The term, “donor nucleotide,” as used herein, refers to a single nucleotide that is incorporated into a target nucleic acid. A nucleotide is typically inserted at a site of cleavage by an effector protein.

The term, “effector protein,” as used herein, refers to a protein, polypeptide, or peptide that non-covalently binds to a guide nucleic acid to form a complex that contacts a target nucleic acid, wherein at least a portion of the guide nucleic acid hybridizes to a target sequence of the target nucleic acid. In some embodiments, the complex between an effector protein and a guide nucleic acid can include multiple effector proteins or a single effector protein. In some embodiments, the effector protein modifies the target nucleic acid when the complex contacts the target nucleic acid. In some embodiments, the effector protein does not modify the target nucleic acid, but it is fused to a fusion partner protein that modifies the target nucleic acid when the complex contacts the target nucleic acid. A non-limiting example of modifying a target nucleic acid is cleaving (hydrolysis) of a phosphodiester bond of the target nucleic acid. Additional examples of modifying target nucleic acids are described herein and throughout.

The term, “functional domain,” as used herein, refers to a region of one or more amino acids in a protein that is required for an activity of the protein, or the full extent of that activity, as measured in an in vitro assay. Activities include, but are not limited to nucleic acid binding, nucleic acid modification, nucleic acid cleavage, protein binding. The absence of the functional domain, including mutations of the functional domain, would abolish or reduce activity.

The term, “functional fragment,” as used herein, refers to a fragment of a protein that retains some function relative to the entire protein. Non-limiting examples of functions are nucleic acid binding, protein binding, nuclease activity, nickase activity, deaminase activity, demethylase activity, or acetylation activity.

The term, “fusion effector protein,” “fusion protein,” and “fusion polypeptide,” as used herein, refer to a protein comprising at least two heterologous polypeptides. Often a fusion effector protein comprises an effector protein and a fusion partner protein. In general, the fusion partner protein is not an effector protein. Examples of fusion partner proteins are provided herein.

The terms, “fusion partner protein” and “fusion partner,” as used herein, refers to a protein, polypeptide or peptide that is fused to an effector protein. The fusion partner generally imparts some function to the fusion protein that is not provided by the effector protein. The fusion partner may provide a detectable signal. The fusion partner may modify a target nucleic acid, including changing a nucleobase of the target nucleic acid and making a chemical modification to one or more nucleotides of the target nucleic acid. The fusion partner may be capable of modulating the expression of a target nucleic acid. The fusion partner may inhibit, reduce, activate, or increase expression of a target nucleic acid via additional proteins or nucleic acid modifications to the target sequence.

“Gene therapy”, as used herein, comprises use of a recombinant nucleic acid (DNA or RNA), administered for the purpose to adjust, repair, replace, add, or remove a gene sequence.

A “genetic disease”, as used herein, refers to a disease, disorder, condition, or syndrome caused by one or more mutations in the DNA of an organism. Mutations can be due to several different cellular mechanisms, including, but not limited to, an error in DNA replication, recombination, or repair, or due to environmental factors. A genetic disease comprises, in some embodiments, a single gene disorder, a chromosome disorder, or a multifactorial disorder.

The term, “guide nucleic acid,” as used herein, refers to at least one nucleic acid comprising: a first nucleotide sequence that hybridizes to a target nucleic acid; and a second nucleotide sequence that is capable of being non-covalently bound by an effector protein. The first sequence may be referred to herein as a spacer sequence. The term, “guide nucleic acid,” may be used to refer to two separate nucleic acids, (e.g., a crRNA and tracrRNA), at least a portion of each hybridize to one another. In some embodiments, the first sequence is covalently linked to the second sequence, either directly (e.g., by a phosphodiester bond) or indirectly (e.g., by one more nucleotides). In some embodiments, the first sequence is located 5′ of the second nucleotide sequence. In some embodiments, the first sequence is located 3′ of the second nucleotide sequence.

The term, “heterologous,” as used herein, means a nucleotide or polypeptide sequence that is not found in a native nucleic acid or protein, respectively. In some embodiments, fusion proteins comprise an effector protein and a fusion partner protein, wherein the fusion partner protein is heterologous to an effector protein. These fusion proteins may be referred to as a “heterologous protein.” A protein that is heterologous to the effector protein is a protein that is not covalently linked via an amide bond to the effector protein in nature. In some embodiments, a heterologous protein is not encoded by a species that encodes the effector protein. In some embodiments, the heterologous protein exhibits an activity (e.g., enzymatic activity) when it is fused to the effector protein. In some embodiments, the heterologous protein exhibits increased or reduced activity (e.g., enzymatic activity) when it is fused to the effector protein, relative to when it is not fused to the effector protein. In some embodiments, the heterologous protein exhibits an activity (e.g., enzymatic activity) that it does not exhibit when it is fused to the effector protein. A guide nucleic acid may comprise a first sequence and a second sequence, wherein the first sequence and the second sequence are not found covalently linked via a phosphodiester bond in nature. Thus, the first sequence is considered to be heterologous with the second sequence, and the guide nucleic acid may be referred to as a heterologous guide nucleic acid.

The term, “in vitro,” as used herein, describes an event that takes places contained in a container for holding laboratory reagents such that it is separated from the biological source from which the material is obtained. In vitro assays can encompass cell-based assays in which living or dead cells are employed. In vitro assays can also encompass a cell-free assay in which no intact cells are employed. The term, “in vivo,” is used to describe an event that takes place in a subject's body. The term, “ex vivo,” is used to describe an event that takes place outside of a subject's body. An ex vivo assay is not performed on a subject. Rather, it is performed upon a sample separate from a subject. An example of an ex vivo assay performed on a sample is an “in vitro” assay.

The term, “linked amino acids,” as used herein, refers to at least two amino acids linked by an amide bond.

The term, “linker,” as used herein, refers to a bond or molecule that links a first polypeptide to a second polypeptide. A “peptide linker” comprises at least two amino acids linked by an amide bond.

The term, “modified target nucleic acid,” as used herein, refers to a target nucleic acid, wherein the target nucleic acid has undergone a modification, for example, after contact with an effector protein. In some cases, the modification is an alteration in the sequence of the target nucleic acid. In some cases, the modified target nucleic acid comprises an insertion, deletion, or replacement of one or more nucleotides compared to the unmodified target nucleic acid.

The term, “mutation associated with a disease,” as used herein, refers to the co-occurrence of a mutation and the phenotype of a disease. The mutation may occur in a gene, wherein transcription or translation products from the gene occur at a significantly abnormal level or in an abnormal form in a cell or subject harboring the mutation as compared to a non-disease control subject not having the mutation.

The terms, “non-naturally occurring” and “engineered,” as used herein, are used interchangeably and indicate the involvement of the hand of man. The terms, when referring to a nucleic acid, nucleotide, protein, polypeptide, peptide or amino acid, refer to a nucleic acid, nucleotide, protein, polypeptide, peptide or amino acid that is at least substantially free from at least one other feature with which it is naturally associated in nature and as found in nature, and/or contains a modification (e.g., chemical modification, nucleotide sequence, or amino acid sequence) that is not present in the naturally occurring nucleic acid, nucleotide, protein, polypeptide, peptide, or amino acid. The terms, when referring to a composition or system described herein, refer to a composition or system having at least one component that is not naturally associated with the other components of the composition or system. By way of a non-limiting example, a composition may include an effector protein and a guide nucleic acid that do not naturally occur together. Conversely, and as a non-limiting further clarifying example, an effector protein or guide nucleic acid that is “natural,” “naturally-occurring,” or “found in nature” includes an effector protein and a guide nucleic acid from a cell or organism that have not been genetically modified by the hand of man.

The term, “nucleic acid expression vector,” as used herein, refers to a plasmid that can be used to express a nucleic acid of interest.

The term, “nuclear localization signal,” as used herein, refers to an entity (e.g., peptide) that facilitates localization of a nucleic acid, protein, or small molecule to the nucleus, when present in a cell that contains a nuclear compartment.

The term, “nuclease activity,” as used herein, refers to the enzymatic activity of an enzyme which allows the enzyme to cleave the phosphodiester bonds between the nucleotide subunits of nucleic acids; the term, “endonuclease activity,” refers to the enzymatic activity of an enzyme which allows the enzyme to cleave the phosphodiester bond within a polynucleotide chain. An enzyme with nuclease activity may be referred to as a “nuclease.”

The terms, “nucleotide” and “nucleoside,” when used in the context of a nucleic acid molecule having multiple residues, are used interchangeably and mean the sugar and base of the residue contained in the nucleic acid molecule. The term, “nucleobase,” when used in the context of a nucleic acid molecule can refer to the base of the residue contained in the nucleic acid molecule, for example, the base of a nucleotide or a nucleoside.

The term, “prime editing enzyme,” as used herein, refers to a protein, polypeptide, or fragment thereof that is capable of catalyzing the modification (insertion, deletion, or base-to-base conversion) of a target nucleotide or nucleotide sequence in a nucleic acid. A prime editing enzyme capable of catalyzing such a reaction includes a reverse transcriptase. A prime editing enzyme may require a prime editing guide RNA (pegRNA) to catalyze the modification. Such a pegRNA can be capable of identifying the nucleotide or nucleotide sequence in the target nucleic acid to be edited and encoding the new genetic information that replaces the targeted nucleotide or nucleotide sequence in the nucleic acid. A prime editing enzyme may require a prime editing guide RNA (pegRNA) and a single guide RNA to catalyze the modification.

The term, “protospacer adjacent motif (PAM),” as used herein, refers to a nucleotide sequence found in a target nucleic acid that directs an effector protein to modify the target nucleic acid at a specific location. A PAM sequence may be required for a complex having an effector protein and a guide nucleic acid to hybridize to and modify the target nucleic acid. However, a given effector protein may not require a PAM sequence being present in a target nucleic acid for the effector protein to modify the target nucleic acid.

The term, “recombinant,” as used herein, as applied to proteins, polypeptides, peptides, and nucleic acids, refers to proteins, polypeptides, peptides and nucleic acids that are products of various combinations of cloning, restriction, and/or ligation steps resulting in a construct having a structural coding or non-coding sequence distinguishable from endogenous nucleic acids found in natural systems. Generally, DNA sequences encoding the structural coding sequence can be assembled from cDNA fragments and short oligonucleotide linkers, or from a series of synthetic oligonucleotides, to provide a synthetic nucleic acid which is capable of being expressed from a recombinant transcriptional unit contained in a cell or in a cell-free transcription and translation system. Such sequences can be provided in the form of an open reading frame uninterrupted by internal non translated sequences, or introns, which are typically present in eukaryotic genes. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions and may act to modulate production of a desired product by various mechanisms (see “DNA regulatory sequences”, below).

The term, “recombinant” polynucleotide or “recombinant” nucleic acid, refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques. The term, “recombinant polypeptide” or “recombinant protein,” refers to a polypeptide which is not naturally occurring, e.g., is made by the artificial combination of two otherwise separated segments of amino sequences through human intervention. Thus, e.g., a polypeptide that comprises a heterologous amino acid sequence is a recombinant polypeptide.

The terms, “reporter,” “reporter nucleic acid” and “reporter molecule,” are used interchangeably herein to refer to a non-target nucleic acid molecule that can provide a detectable signal upon cleavage by an effector protein. Examples of detectable signals and detectable moieties that generate detectable signals are provided herein.

The term, “sample,” as used herein, generally refers to something comprising a target nucleic acid. In some embodiments, the sample is a biological sample, such as a biological fluid or tissue sample. In some embodiments, the sample is an environmental sample. The sample may be a biological sample or environmental sample that is modified or manipulated. By way of non-limiting example, samples may be modified or manipulated with purification techniques, heat, nucleic acid amplification, salts, and buffers.

The term, “subject,” as used herein, can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some embodiments, the subject is not necessarily diagnosed or suspected of being at high risk for the disease.

A “syndrome”, as used herein, refers to a group of symptoms which, taken together, characterize a condition.

The term, “target nucleic acid,” as used herein, refers to a nucleic acid that is selected as the nucleic acid for modification, binding, hybridization, or any other activity of or interaction with a nucleic acid, protein, polypeptide, or peptide described herein. A target nucleic acid may comprise RNA, DNA, or a combination thereof. A target nucleic acid may be single-stranded (e.g., single-stranded RNA or single-stranded DNA) or double-stranded (e.g., double-stranded DNA).

The term, “target sequence,” as used herein, when used in reference to a target nucleic acid, refers to a sequence of nucleotides that hybridizes to an equal length portion of a guide nucleic acid. Hybridization of the guide nucleic acid to the target sequence may bring an effector protein into contact with the target nucleic acid.

The term, “trans cleavage,” is used herein in reference to cleavage (hydrolysis of a phosphodiester bond) of one or more nucleic acids by an effector protein that is complexed with a guide nucleic acid and a target nucleic acid. The one or more nucleic acids may include the target nucleic acid as well as non-target nucleic acids.

The term, “trans-activating RNA (tracrRNA),” as used herein, refers to a nucleic acid that comprises a first sequence that is capable of being non-covalently bound by an effector protein. TracrRNAs may comprise a second sequence that hybridizes to a portion of a crRNA, which may be referred to as a repeat sequence. In some embodiments, tracrRNAs are covalently linked to a crRNA.

The term, “transcriptional activator,” as used herein, refers to a polypeptide or a fragment thereof that can activate or increase transcription of a target nucleic acid molecule.

The term, “transcriptional repressor,” as used herein, refers to a polypeptide or a fragment thereof that is capable of arresting, preventing, or reducing transcription of a target nucleic acid.

The terms, “treatment” and “treating,” as used herein, are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying, or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The term, “viral vector,” as used herein, refers to a nucleic acid to be delivered into a host cell via a recombinantly produced virus or viral particle. The nucleic acid may be single-stranded or double stranded, linear or circular, segmented or non-segmented. The nucleic acid may comprise DNA, RNA, or a combination thereof. Non-limiting examples of viruses or viral particles that can deliver a viral vector include retroviruses (e.g., lentiviruses and γ-retroviruses), adenoviruses, arenaviruses, alphaviruses, adeno-associated viruses (AAVs), baculoviruses, vaccinia viruses, herpes simplex viruses and poxviruses. A viral vector delivered by such viruses or viral particles may be referred to by the type of virus to deliver the viral vector (e.g., an AAV viral vector is a viral vector that is to be delivered by an adeno-associated virus). A viral vector referred to by the type of virus to be delivered by the viral vector can contain viral elements (e.g., nucleotide sequences) necessary for packaging of the viral vector into the virus or viral particle, replicating the virus, or other desired viral activities. A virus containing a viral vector may be replication competent, replication deficient or replication defective.

Disclosed herein are non-naturally occurring compositions, methods and systems comprising an effector protein and an engineered guide nucleic acid, which may simply be referred to herein as a guide nucleic acid. Also disclosed herein are non-naturally occurring compositions, methods and systems comprising a nucleic acid encoding an effector protein and nucleic acid encoding a guide nucleic acid. In general, an engineered effector protein and an engineered guide nucleic acid refer to an effector protein and a guide nucleic acid, respectively, that are not found in nature. In some embodiments, systems, methods and compositions comprise at least one non-naturally occurring component. For example, compositions, methods and systems may comprise a guide nucleic acid, wherein the sequence of the guide nucleic acid is different or modified from that of a naturally-occurring guide nucleic acid.

In some embodiments, compositions, methods and systems comprise at least two components that do not naturally occur together. For example, compositions, methods and systems may comprise a guide nucleic acid comprising a repeat sequence and a spacer sequence which do not naturally occur together. Also, by way of example, composition, methods and systems may comprise a guide nucleic acid and an effector protein that do not naturally occur together. Also, by way of non-limiting example, disclosed compositions, systems, and methods may comprise a guide nucleic acid and an effector protein that do not naturally occur together. Likewise, by way of non-limiting example, disclosed compositions, systems, and methods may comprise a ribonucleotide-protein (RNP) complex comprising an effector protein and a guide nucleic acid that do not occur together in nature. Conversely, and for clarity, an effector protein or guide nucleic acid that is “natural,” “naturally-occurring,” or “found in nature” includes effector proteins and guide nucleic acids from cells or organisms that have not been genetically modified by a human or machine.

In some embodiments, a guide nucleic acid comprises: a first nucleotide sequence that hybridizes to a target nucleic acid; and a second nucleotide sequence that is capable of being connected to an effector protein by, for example, being non-covalently bound by an effector protein or hybridized to a separate nucleic acid molecule that is bound by an effector protein. The first sequence may be referred to herein as a spacer sequence. The second sequence may be referred to herein as a repeat sequence. In some instances, the first sequence is located 5′ of the second nucleotide sequence. In some instances, the first sequence is located 3′ of the second nucleotide sequence. In some embodiments, the guide nucleic acid comprises a non-natural nucleobase sequence. In some embodiments, the non-natural sequence is a nucleobase sequence that is not found in nature. The non-natural sequence may comprise a portion of a naturally-occurring sequence, wherein the portion of the naturally-occurring sequence is not present in nature, absent the remainder of the naturally-occurring sequence. In some embodiments, the guide nucleic acid comprises two naturally-occurring sequences arranged in an order or proximity that is not observed in nature. In some embodiments, compositions, methods and systems comprise a ribonucleotide complex comprising an effector protein and a guide nucleic acid that do not occur together in nature. Engineered guide nucleic acids may comprise a first sequence and a second sequence that do not occur naturally together. For example, an engineered guide nucleic acid may comprise a sequence of a naturally-occurring repeat sequence and a spacer sequence that is complementary to a naturally-occurring eukaryotic sequence. The engineered guide nucleic acid may comprise a sequence of a repeat sequence that occurs naturally in an organism and a spacer sequence that does not occur naturally in that organism. An engineered guide nucleic acid may comprise a first sequence that occurs in a first organism and a second sequence that occurs in a second organism, wherein the first organism and the second organism are different. The guide nucleic acid may comprise a third sequence located at a 3′ or 5′ end of the guide nucleic acid, or between the first and second sequences of the guide nucleic acid. For example, an engineered guide nucleic acid may comprise a naturally occurring CRISPR RNA (crRNA) and trans-activating crRNA (tracrRNA) sequence coupled by a linker sequence (e.g., sgRNA).

In some embodiments, compositions, methods and systems described herein comprise an engineered effector protein that is similar to a naturally occurring effector protein. The engineered effector protein may lack a portion of the naturally occurring effector protein. The effector protein may comprise a mutation relative to the naturally-occurring effector protein, wherein the mutation is not found in nature. The effector protein may also comprise at least one additional amino acid relative to the naturally-occurring effector protein. For example, the effector protein may comprise an addition of a nuclear localization signal relative to the natural occurring effector protein. In certain embodiments, the nucleotide sequence encoding the effector protein is codon optimized (e.g., for expression in a eukaryotic cell) relative to the naturally occurring sequence.

### I. Effector Proteins

Provided herein, in certain embodiments, are compositions that comprise one or more effector proteins and uses thereof. Also provided herein are compositions that comprise a nucleic acid, wherein the nucleic acid encodes any of one the effector proteins described herein. The nucleic acid may be a nucleic acid expression vector. By way of non-limiting example, the nucleic acid expression vector may be a viral vector, such as an AAV vector. In general, effector proteins disclosed herein are CRISPR-associated (“Cas”) proteins.

An effector protein provided herein interacts with a guide nucleic acid to form a complex. In some embodiments, the complex interacts with a target nucleic acid, a non-target nucleic acid, or both. In some embodiments, an interaction between the complex and a target nucleic acid, a non-target nucleic acid, or both comprises one or more of: recognition of a protospacer adjacent motif (PAM) sequence within the target nucleic acid by the effector protein, hybridization of the guide nucleic acid to the target nucleic acid, modification of the target nucleic acid and/or the non-target nucleic acid by the effector protein, or combinations thereof. In some embodiments, recognition of a PAM sequence within a target nucleic acid may direct the modification activity of an effector protein.

In some embodiments, the effector protein described herein may bind and, optionally, modify nucleic acids in a sequence-specific manner. Effector proteins described herein may also modify the target nucleic acid within a target sequence or at a position adjacent to the target sequence. In some embodiments, an effector protein is activated when it binds a certain sequence of a nucleic acid described herein, allowing the effector protein to modify a region of a target nucleic acid that is near, but not adjacent to the target sequence. Effector proteins may modify a nucleic acid by cis cleavage or trans cleavage. Alternatively or additionally, effector proteins described herein modify a non-target nucleic acid by trans cleavage on the non-target nucleic acid. In some embodiments, effector proteins may have nickase activity or nuclease activity. Effector proteins disclosed herein may modify nucleic acids, including single stranded RNA (ssRNA), double stranded DNA (dsDNA), and single-stranded DNA (ssDNA). The modification of the target nucleic acid generated by an effector protein, as a non-limiting example, may result in modulation of the expression of the nucleic acid (e.g., increasing or decreasing expression of the nucleic acid) or modulation of the activity of a translation product of the target nucleic acid (e.g., inactivation of a protein binding to an RNA molecule or hybridization).

An effector protein may be a CRISPR-associated (“Cas”) protein. An effector protein may function as a single protein, including a single protein that is capable of binding to a guide nucleic acid and modifying a target nucleic acid. Alternatively, an effector protein may function as part of a multiprotein complex, including, for example, a complex having two or more effector proteins, including two or more of the same effector proteins (e.g., dimer or multimer). In some embodiments, an effector protein, when functioning in a multiprotein complex, may have differing and/or complementary functional activity to other effector proteins in the multiprotein complex. An effector protein, when functioning in a multiprotein complex, may have only one functional activity (e.g., binding to a guide nucleic acid), while other effector proteins present in the multiprotein complex are capable of the other functional activity (e.g., modifying a target nucleic acid). The first and second effector proteins may be the same. The first and second effector proteins may be different. The sequences of the first and second effector proteins may be 15% to 20% identical, 20% to 25% identical, 25% to 30% identical, 30% to 35% identical, 35% to 40% identical, 40% to 45% identical, 45% to 50% identical, 50% to 55% identical, 55% to 60% identical, 60% to 65% identical, 65% to 70% identical, 70% to 75% identical, 75% to 80% identical, 80% to 85% identical, 85% to 90% identical, 90% to 95% identical, 95% to 99.9% identical, or 100% identical.

An effector protein may be a modified effector protein having increased modification activity (e.g., catalytic activity) and/or increased substrate binding activity (e.g., substrate selectivity, specificity, and/or affinity). An effector protein may be a modified effector protein having reduced modification activity (e.g., a catalytically defective effector protein) or no modification activity (e.g., a catalytically inactive effector protein). Accordingly, an effector protein as used herein encompasses a modified or programmable nuclease that does not have nuclease activity.

In some embodiments, effector proteins described herein comprise one or more functional domains. Effector protein functional domains can include a protospacer adjacent motif (PAM)-interacting domain, an oligonucleotide-interacting domain, one or more recognition domains, a non-target strand interacting domain, and a RuvC domain. A PAM interacting domain can be a target strand PAM interacting domain (TPID) or a non-target strand PAM interacting domain (NTPID). In some embodiments, a PAM interacting domain, such as a TPID or a NTPID, on an effector protein describes a region of an effector protein that interacts with target nucleic acid. In some embodiments, the effector proteins comprise a RuvC domain. In some embodiments, a RuvC domain comprises with substrate binding activity, catalytic activity, or both. In some embodiments, the RuvC domain may be defined by a single, contiguous sequence, or a set of RuvC subdomains that are not contiguous with respect to the primary amino acid sequence of the protein. An effector protein of the present disclosure may include multiple RuvC subdomains, which may combine to generate a RuvC domain with substrate binding or catalytic activity. For example, an effector protein may include three RuvC subdomains (RuvC-I, RuvC-II, and RuvC-III) that are not contiguous with respect to the primary amino acid sequence of the effector protein but form a RuvC domain once the protein is produced and folds. In some embodiments, effector proteins comprise one or more recognition domain (REC domain) with a binding affinity for a guide nucleic acid or for a guide nucleic acid-target nucleic acid heteroduplex. An effector protein may comprise a zinc finger domain. In some embodiments, the effector protein does not comprise an HNH domain.

In some embodiments, the effector protein may have a mutation in a nuclease domain. In some embodiments, the nuclease domain is a RuvC domain. In some embodiments, the nuclease domain is an HNH domain. An HNH domain may be characterized as comprising two antiparallel β-strands connected with a loop of varying length, and flanked by an α-helix, with a metal (divalent cation) binding site between the two β-strands. A RuvC domain may be characterized by a six-stranded beta sheet surrounded by four alpha helices, with three conserved subdomains contributing catalytic to the activity of the RuvC domain.

When describing a mutation that changes an amino acid residue or a nucleotide as described herein, such a change or changes can include, for example, deletions, insertions, and/or substitutions. The mutation can refer to a change in structure of an amino acid residue or nucleotide relative to the starting or reference residue or nucleotide. A mutation of an amino acid residue includes, for example, deletions, insertions and substituting one amino acid residue for a structurally different amino acid residue. Such substitutions can be a conservative substitution, a non-conservative substitution, a substitution to a specific sub-class of amino acids, or a combination thereof as described herein. A mutation of a nucleotide includes, for example, changing one naturally occurring base for a different naturally occurring base, such as changing an adenine to a thymine or a guanine to a cytosine or an adenine to a cytosine or a guanine to a thymine. A mutation of a nucleotide base may result in a structural and/or functional alteration of the encoding peptide, polypeptide or protein by changing the encoded amino acid residue of the peptide, polypeptide or protein. A mutation of a nucleotide base may not result in an alteration of the amino acid sequence or function of encoded peptide, polypeptide or protein, also known as a silent mutation.

When a conservative substitution is described herein, such a substitution refers to the replacement of one amino acid for another such that the replacement takes place within a family of amino acids that are related in their side chains. Alternatively, a non-conservative substitution, when described herein, refers to the replacement of one amino acid residue for another such that the replaced residue is going from one family of amino acids to a different family of residues. Genetically encoded amino acids can be divided into four families: (1) acidic (negatively charged)=Asp (D), Glu (G); (2) basic (positively charged)=Lys (K), Arg (R), His (H); (3) non-polar (hydrophobic)=Cys (C), Ala (A), Val (V), Leu (L), Ile (I), Pro (P), Phe (F), Met (M), Trp (W), Gly (G), Tyr (Y), with non-polar also being subdivided into: (i) strongly hydrophobic=Ala (A), Val (V), Leu (L), Ile (I), Met (M), Phe (F); and (ii) moderately hydrophobic=Gly (G), Pro (P), Cys (C), Tyr (Y), Trp (W); and (4) uncharged polar=Asn (N), Gln (Q), Ser (S), Thr (T). In alternative fashion, the amino acid repertoire can be grouped as (1) acidic (negatively charged)=Asp (D), Glu (G); (2) basic (positively charged)=Lys (K), Arg (R), His (H), and (3) aliphatic=Gly (G), Ala (A), Val (V), Leu (L), Ile (I), Ser (S), Thr (T), with Ser (S) and Thr (T) optionally being grouped separately as aliphatic-hydroxyl; (4) aromatic=Phe (F), Tyr (Y), Trp (W); (5) amide=Asn (N), Glu (Q); and (6) sulfur-containing=Cys (C) and Met (M) (see, for example, Biochemistry, 4th ed., Ed. by L. Stryer, WH Freeman and Co., 1995, which is incorporated by reference herein in its entirety).

An effector protein may be brought into proximity of a target nucleic acid in the presence of a guide nucleic acid when the guide nucleic acid includes a nucleotide sequence that is complementary with a target sequence in the target nucleic acid. The ability of an effector protein to modify a target nucleic acid may be dependent upon the effector protein being bound to a guide nucleic acid and the guide nucleic acid being hybridized to a target nucleic acid. An effector protein may recognize a PAM sequence present in the target nucleic acid, which may direct the modification activity of the effector protein. In some embodiments, effector proteins described herein may provide blunt or short stagger ends. Blunt cutting may be advantageous over the staggered cutting that is provided by other effector proteins, as there is a less likely chance of spontaneous (also referred to as perfect) repair which may decrease the chances of successful target nucleic acid editing and/or donor nucleic acid insertion.

TABLE 1 provides illustrative amino acid sequences of effector proteins. In some embodiments, the effector protein comprises an amino acid sequence that is at least 65%, at least 70%, at least 75%, at least 80%, at least 85%, at least 90%, at least 95%, at least 97%, or at least 98%, at least 99%, or 100% identical to any one of SEQ ID NOS: 1-78 and 499. In some embodiments, the amino acid sequence of the effector protein is at least 65%, at least 70%, at least 75%, at least 80%, at least 85%, at least 90%, at least 95%, at least 97%, or at least 98%, at least 99%, or 100% identical to any one of SEQ ID NOS: 1-78 and 499.

In some embodiments, compositions comprise an effector protein, wherein the effector protein comprises an amino acid sequence that is at least 65%, at least 70%, at least 75%, at least 80%, at least 85%, at least 90%, at least 95%, at least 97%, or at least 98%, at least 99%, or 100% identical to any one of the sequences recited in TABLE 1. In some embodiments, compositions comprise an effector protein, wherein the amino acid sequence of the effector protein is at least 65%, at least 70%, at least 75%, at least 80%, at least 85%, at least 90%, at least 95%, at least 97%, or at least 98%, at least 99%, or 100% identical to any one of the sequences recited in TABLE 1. In some embodiments, the amino acid sequence is at least 80% identical to any one of the sequences recited in TABLE 1. In some embodiments, the amino acid sequence is at least 85% identical to any one of the sequences recited in TABLE 1. In some embodiments, the amino acid sequence is at least 90% identical to any one of the sequences recited in TABLE 1. In some embodiments, the amino acid sequence is at least 95% identical to any one of the sequences recited in TABLE 1. In some embodiments, the amino acid sequence is at least 97% identical to any one of the sequences recited in TABLE 1. In some embodiments, the amino acid sequence is at least 99% identical to any one of the sequences recited in TABLE 1. In some embodiments, the amino acid sequence is 100% identical to any one of the sequences recited in TABLE 1.

In some embodiments, compositions, systems and methods described herein comprise an effector protein, or a nucleic acid encoding the effector protein, wherein the amino acid sequence of the effector protein comprises at least about 200 contiguous amino acids or more of any one of the sequences recited in Error! Reference source not found. In some embodiments, the amino acid sequence of an effector protein provided herein comprises at least about 200, at least about 220, at least about 240, at least about 260, at least about 280, at least about 300, at least about 320, at least about 340, at least about 360, at least about 380, at least about 400 contiguous amino acids, at least about 420 contiguous amino acids, at least about 440 contiguous amino acids, at least about 460 contiguous amino acids, at least about 480 contiguous amino acids, at least about 500 contiguous amino acids, at least about 520 contiguous amino acids, at least about 540 contiguous amino acids, at least about 560 contiguous amino acids, at least about 580 contiguous amino acids, at least about 600 contiguous amino acids, at least about 620 contiguous amino acids, at least about 640 contiguous amino acids, at least about 660 contiguous amino acids, at least about 680 contiguous amino acids, at least about 700 contiguous amino acids, or more of any one of the sequences of Error! Reference source not found.

In some embodiments, compositions, systems and methods described herein comprise an effector protein or a nucleic acid encoding the effector protein, wherein the effector protein comprises a portion of any one of the sequences recited in TABLE 1. In some embodiments, the effector protein comprises a portion of any one of the sequences recited in TABLE 1, wherein the portion does not comprise at least the first 10 amino acids, at least the first 20 amino acids, at least the first 40 amino acids, at least the first 60 amino acids, at least the first 80 amino acids, at least the first 100 amino acids, at least the first 120 amino acids, at least the first 140 amino acids, at least the first 160 amino acids, at least the first 180 amino acids, or at least the first 200 amino acids of any one of the sequences recited in TABLE 1. In some embodiments, the effector protein comprises a portion of any one of the sequences recited in TABLE 1, wherein the portion does not comprise the last 10 amino acids, the last 20 amino acids, the last 40 amino acids, the last 60 amino acids, the last 80 amino acids, the last 100 amino acids, the last 120 amino acids, the last 140 amino acids, the last 160 amino acids, the last 180 amino acids, or the last 200 amino acids of any one of the sequences recited in TABLE 1.

In some embodiments, compositions comprise an effector protein, wherein portion of the amino acid sequence of the effector protein is at least 65%, at least 70%, at least 75%, at least 80%, at least 85%, at least 90%, at least 95%, at least 97%, or at least 98%, at least 99%, or 100% identical to an equal length portion of a sequence selected from SEQ ID NOs: 1-78 and 499. In some embodiments, the length of the portion is selected from: 20 to 40, 40 to 60, 60 to 80, 80 to 100, 100 to 120, 120 to 140, 140 to 160, 160 to 180, 180 to 200, 200 to 220, 220 to 240, 240 to 260, 260 to 280, 280 to 300, 320 to 340, 340 to 360, 360 to 380, and 380 to 400 linked amino acids. In some embodiments, the length of the portion is selected from: 400 to 420, 420 to 440, 440 to 460, 460 to 480, 480 to 500, 520 to 540, 540 to 560, 560 to 580, 580 to 600, 600 to 620, 620 to 640, 640 to 660, 660 to 680, and 680 to 700, 700 to 720, 720 to 740, 740 to 760, 760 to 780, 780 to 800, 820 to 840, 840 to 860, 860 to 880, 880 to 900, 900 to 920, 920 to 940, 940 to 960, 960 to 980, and 980 to 1000 linked amino acids. In some embodiments, the length of the portion is selected from: 1000 to 1020, 1020 to 1040, 1040 to 1060, 1060 to 1080, 1080 to 1100, 1100 to 1120, 1120 to 1140, 1140 to 1160, 1160 to 1180, 1180 to 1200, 1220 to 1240, 1240 to 1260, 1260 to 1280, 1280 to 1300, 1300 to 1320, 1320 to 1340, 1340 to 1360, 1360 to 1380, 1380 to 1400, 1420 to 1440, 1440 to 1460, 1460 to 1480, and 1480 to 1500 linked amino acids.

In some embodiments, compositions, systems, and methods described herein comprise an effector protein, or a nucleic acid encoding the effector protein, wherein the effector protein comprises an amino acid sequence that is at least 80%, at least 85%, at least 90%, at least 95%, at least 97%, at least 98%, at least 99%, or 100% similar to any one of the sequences as set forth in TABLE 1. In some embodiments, an effector protein provided herein comprises an amino acid sequence that is at least 80% similar to any one of the sequences as set forth in TABLE 1. In some embodiments, an effector protein provided herein comprises an amino acid sequence that is at least 85% similar to any one of the sequences as set forth in TABLE 1. In some embodiments, an effector protein provided herein comprises an amino acid sequence that is at least 90% identical to any one of the sequences as set forth in TABLE 1. In some embodiments, an effector protein provided herein comprises an amino acid sequence that is at least 95% similar to any one of the sequences as set forth in TABLE 1. In some embodiments, an effector protein provided herein comprises an amino acid sequence that is at least 97% similar to any one of the sequences as set forth in TABLE 1. In some embodiments, an effector protein provided herein comprises an amino acid sequence that is at least 98% identical to any one of the sequences as set forth in TABLE 1. In some embodiments, an effector protein provided herein comprises an amino acid sequence that is at least 99% similar to any one of the sequences as set forth in TABLE 1. In some embodiments, an effector protein provided herein comprises an amino acid sequence that is 100% similar to any one of the sequences as set forth in TABLE 1.

In some embodiments, when describing a certain percent (%) similarity in the context of an amino acid sequence, reference may be made to a value that is calculated by dividing a similarity score by the length of the alignment. In some embodiments, the similarity of two amino acid sequences can be calculated by using a BLOSUM62 similarity matrix (Henikoff and Henikoff, Proc. Natl. Acad. Sci. USA., 89:10915-10919 (1992)) that is transformed so that any value >1 is replaced with +1 and any value <0 is replaced with 0. For example, an Ile (I) to Leu (L) substitution is scored at +2.0 by the BLOSUM62 similarity matrix, which in the transformed matrix is scored at +1. This transformation allows the calculation of percent similarity, rather than a similarity score. Alternately, in some embodiments, when comparing two full protein sequences, the proteins can be aligned using pairwise MUSCLE alignment. Then, the % similarity can be scored at each residue and divided by the length of the alignment. For determining % similarity over a protein domain or motif, a multilevel consensus sequence (or PROSITE motif sequence) can be used to identify how strongly each domain or motif is conserved. In calculating the similarity of a domain or motif, the second and third levels of the multilevel sequence are treated as equivalent to the top level. Additionally, in some embodiments, if a substitution could be treated as conservative with any of the amino acids in that position of the multilevel consensus sequence, +1 point is assigned. For example, given the multilevel consensus sequence: RLG and YCK, the test sequence QIQ would receive three points. This is because in the transformed BLOSUM62 matrix, each combination is scored as: Q-R: +1; Q-Y: +0; I-L: +1; I-C: +0; Q-G: +0; Q-K: +1. For each position, the highest score is used when calculating similarity. In some embodiments, the % similarity can also be calculated using commercially available programs, such as the Geneious Prime software given the parameters matrix=BLOSUM62 and threshold >1.

In some embodiments, compositions, systems, and methods described herein comprise an effector protein, or a nucleic acid encoding the effector protein, wherein the effector protein comprises one or more amino acid alterations relative to any one of the sequences recited in TABLE 1. In some embodiments, the effector protein comprising one or more amino acid alterations is a variant of an effector protein described herein. It is understood that any reference to an effector protein herein also refers to an effector protein variant as described herein. In some embodiments, the one or more amino acid alterations comprises conservative substitutions, non-conservative substitutions, conservative deletions, non-conservative deletions, or combinations thereof. In some embodiments, an effector protein or a nucleic acid encoding the effector protein comprises 1 amino acid alteration, 2 amino acid alterations, 3 amino acid alterations, 4 amino acid alterations, 5 amino acid alterations, 6 amino acid alterations, 7 amino acid alterations, 8 amino acid alterations, 9 amino acid alterations, 10 amino acid alterations or more relative to any one of the sequences recited in TABLE 1.

In some embodiments, effector proteins comprise a functional domain. The functional domain may comprise nucleic acid binding activity. The functional domain may comprise catalytic activity, also referred to as enzymatic activity. The catalytic activity may be nuclease activity. The nuclease activity may comprise cleaving a strand of a nucleic acid. The nuclease activity may comprise cleaving only one strand of a double stranded nucleic acid, also referred to as nicking. In some embodiments, the functional domain is an HNH domain. In some embodiments, the functional domain is a RuvC domain. In some embodiments, the RuvC domain comprises multiple subdomains. In some embodiments, the functional domain is a zinc finger binding domain. In some embodiments, effector proteins lack a certain functional domain. In some embodiments, the effector protein lacks an HNH domain. In some embodiments, effector proteins lack a zinc finger binding domain. In some embodiments, the effector protein lacks a HEPN domain.

In some embodiments, effector proteins catalyze cleavage of a target nucleic acid in a cell or a sample. In some embodiments, the target nucleic acid is single stranded (ss). In some embodiments, the target nucleic acid is double stranded (ds). In some embodiments, the target nucleic acid is dsDNA. In some embodiments, the target nucleic acid is ssDNA. In some embodiments, the target nucleic acid is RNA. In some embodiments, effector proteins cleave the target nucleic acid within a target sequence of the target nucleic acid. In some embodiments, effector proteins cleave the target nucleic acid, as well as additional nucleic acids in the cell or the sample, which may be referred to as trans cleavage activity or simply trans cleavage. In some embodiments, trans cleavage may occur near, but not within or directly adjacent to, a target nucleic acid that is hybridized to a guide nucleic acid. Trans cleavage activity may be triggered by the hybridization of the guide nucleic acid to the target nucleic acid.

In some embodiments, cleaving a nucleic acid molecule includes hydrolysis of a phosphodiester bond of a nucleic acid molecule resulting in breakage of that bond. In some embodiments, the breakage is a nick (hydrolysis of a single phosphodiester bond on one side of a double-stranded molecule), single strand break (hydrolysis of a single phosphodiester bond on a single-stranded molecule) or double strand break (hydrolysis of two phosphodiester bonds on both sides of a double-stranded molecule) depending upon whether the nucleic acid molecule is single-stranded (e.g., ssDNA or ssRNA) or double-stranded (e.g., dsDNA) and the type of nuclease activity being catalyzed by the effector protein.

In some embodiments, effector proteins catalyze cis cleavage activity. In some embodiments, effector proteins cleave both strands of dsDNA.

### Engineered Proteins

In some embodiments, effector proteins disclosed herein are engineered proteins. Engineered proteins are not identical to a naturally-occurring protein. Such an engineered protein can include one or more mutations, including an insertion, deletion or substitution (e.g., conservative or non-conservative substitution). An engineered protein, in some embodiments, includes at least one mutation relative to a reference protein (e.g., a naturally-occurring protein). In some embodiments, an engineered protein includes at least 1, at least 2, at least 3, at least 4, at least 5, at least 6, at least 7, at least 8, at least 9, at least 10, at least 15, at least 20, at least 25 or at least 30 mutations relative to a reference protein (e.g., a naturally-occurring protein). In some embodiments, an engineered protein includes no more than 10, 20, 30, 40, or 50 mutations relative to a reference protein (e.g., a naturally-occurring protein). Engineered proteins may not comprise an amino acid sequence that is identical to that of a naturally-occurring protein. In some embodiments, the amino acid sequence of an engineered protein is not identical to that of a naturally occurring protein. Engineered proteins may provide an increased activity relative to a naturally occurring protein. Engineered proteins may provide a reduced activity relative to a naturally occurring protein. The activity may be nuclease activity. The activity may be nickase activity. The activity may be nucleic acid binding activity. Accordingly, in some embodiments, engineered proteins may provide enhanced activity (e.g., enhanced binding of a guide nucleic acid, and/or target nucleic acid, enhanced nuclease activity, enhanced nickase activity, etc.) as compared to a naturally-occurring counterpart. In such embodiments, the effector protein may have a 1%, 5%, 10%, 20%, 30%, 40%, 50%, 60%, 70%, 80%, 90%, 100%, 120%, 140%, 160%, 180%, 200%, or more, increased activity relative to a naturally-occurring counterpart. Alternatively, in some embodiments, engineered proteins may provide reduced activity (e.g., reduced binding of a guide nucleic acid, and/or target nucleic acid, reduced nuclease activity, reduced nickase activity, etc.) relative to a naturally occurring effector protein. In such embodiments, the engineered proteins may have a 100%, 90%, 80%, 70%, 60%, 50%, 40%, 30%, 20%, 10%, 5%, 1%, or less, decreased activity relative to a naturally occurring counterpart.

An effector protein that has decreased catalytic activity may be referred to as catalytically or enzymatically inactive, catalytically or enzymatically dead, as a dead protein or a dCas protein. In some embodiments, such a protein may comprise an enzymatically inactive domain (e.g., inactive nuclease domain). For example, a nuclease domain (e.g., RuvC domain) of an effector protein may be deleted or mutated relative to a wildtype counterpart so that it is no longer functional or comprises reduced nuclease activity.

In some embodiments, a catalytically inactive effector protein may bind to a guide nucleic acid and/or a target nucleic acid but does not cleave the target nucleic acid. In some embodiments, a catalytically inactive effector protein may associate with a guide nucleic acid to activate or repress transcription of a target nucleic acid. In some embodiments, a catalytically inactive effector protein is fused to a fusion partner protein that confers an alternative activity to an effector protein activity. Such fusion proteins are described herein and throughout.

Engineered proteins may provide an increased or reduced activity relative to a naturally occurring protein under a given condition of a cell or sample in which the activity occurs. The condition may be temperature. The temperature may be greater than 20° C., greater than 25° C., greater than 30° C., greater than 35° C., greater than 40° C., greater than 45° C., greater than 50° C., greater than 55° C., greater than 60° C., greater than 65° C., or greater than 70° C., but not greater than 80° C. The condition may be the presence of a salt. The salt may be a magnesium salt, a zinc salt, a potassium salt, a calcium salt, or a sodium salt. The condition may be the concentration of one or more salts.

In some embodiments, the amino acid sequence of an engineered protein comprises at least one residue that is different from that of a naturally occurring protein. In some embodiments, the amino acid sequence of an engineered protein comprises at least 2, at least 3, at least 4, at least 5, at least 6, at least 7, at least 8, at least 9 or at least 10 residues that are different from that of a naturally occurring protein. The residues in the engineered protein that differ from those at corresponding positions of the naturally occurring protein (when the engineered and naturally occurring proteins are aligned for maximal identity) may be referred to as substituted residues or amino acid substitutions. In some embodiments, the substituted residues are non-conserved residues relative to the residues at corresponding positions of the naturally occurring protein. A non-conserved residue has a different physicochemical property from the amino acid for which it substitutes. Physicochemical properties include aliphatic, cyclic, aromatic, basic, acidic and hydroxyl-containing. Glycine, alanine, valine, leucine, and isoleucine are aliphatic. Serine, Cysteine, threonine, and methionine are hydroxyl-containing. Proline is cyclic. Phenylalanine, tyrosine, tryptophan are basic. Aspartate, Glutamate, Asparagine, and glutamine are acidic.

In some embodiments, engineered proteins are designed to be catalytically inactive or to have reduced catalytic activity relative to a naturally occurring protein. A catalytically inactive effector protein may be generated by substituting an amino acid that confers a catalytic activity (also referred to as a “catalytic residue”) with a substituted residue that does not support the catalytic activity. In some embodiments, the substituted residue has an aliphatic side chain. In some embodiments, the substituted residue is glycine. In some embodiments, the substituted residue is valine. In some embodiments, the substituted residue is leucine. In some embodiments, the substituted residue is alanine. In some embodiments, the amino acid is aspartate and it is substituted with asparagine. In some embodiments, the amino acid is glutamate and it is substituted with glutamine. An amino acid that confers catalytic activity may be identified by performing sequence alignment of an unmodified effector protein with a similar enzyme having at least one identified catalytic residue; selecting at least one putative catalytic residue in the unmodified effector protein within the portion of the unmodified effector protein that aligns with a portion of the similar enzyme that comprises the identified catalytic residue; substituting the at least one putative catalytic residue of the unmodified effector protein with the different amino acid; and comparing the catalytic activity of the unmodified effector protein to the modified effector protein. A similar enzyme may be an enzyme that is at least 50%, at least 60%, at least 70%, at least 80%, or at least 90% identical to the unmodified effector protein. A similar enzyme may be an enzyme that is not greater than 99.9% identical to the unmodified effector protein. In some embodiments, the portion of the unmodified effector protein that aligns with a portion of the similar enzyme is at least 10 amino acids, at least 20 amino acids, at least 30 amino acids, at least 40 amino acids, at least 50 amino acids, at least 60 amino acids, at least 70 amino acids, at least 80 amino acids, at least 90 amino acids, or at least 100 amino acids in length. In some embodiments, the portion of the unmodified effector protein that aligns with a portion of the similar enzyme is not greater than 200 amino acids. In some embodiments, the portion of the unmodified effector protein that aligns with a portion of the similar enzyme comprises a functional domain (e.g., HNH, RuvC, zinc finger binding). In some embodiments, comparing the catalytic activity comprises performing a cleavage assay. An example of generating a catalytically inactive effector protein is provided in Example 7.

In some embodiments, effector proteins described herein are encoded by a codon optimized nucleic acid. In some embodiments, a nucleic acid sequence encoding an effector protein described herein is codon optimized. A nucleic acid encoding an effector protein may be codon optimized for expression in a specific cell, for example, a bacterial cell, a plant cell, a eukaryotic cell, an animal cell, a mammalian cell, or a human cell. This type of optimization can entail a mutation of an effector protein encoding nucleotide sequence to mimic the codon preferences of the intended host organism or cell while encoding the same polypeptide. Thus, the codons can be changed, but the encoded protein remains unchanged. For example, if the intended target cell was a human cell, a human codon-optimized effector protein-encoding nucleotide sequence could be used. Accordingly, in some embodiments, the nucleic acid encoding an effector protein is codon optimized for a human cell. As another non-limiting example, if the intended host cell were a mouse cell, then a mouse codon-optimized effector protein-encoding nucleotide sequence could be generated. As another non-limiting example, if the intended host cell were a eukaryotic cell, then a eukaryote codon-optimized effector protein-encoding nucleotide sequence could be generated. As another non-limiting example, if the intended host cell were a prokaryotic cell, then a prokaryote codon-optimized effector protein-encoding nucleotide sequence could be generated. Codon usage tables are readily available, for example, at the “Codon Usage Database” available at www.kazusa.or.jp/codon.

It is understood that when describing coding sequences of polypeptides described herein, said coding sequences do not necessarily require a codon encoding a N-terminal Methionine (M) or a Valine (V) as described for the effector proteins described herein. One skilled in the art would understand that a start codon could be replaced or substituted with a start codon that encodes for an amino acid residue sufficient for initiating translation in a host cell.

### Fusion Proteins

In some embodiments, compositions comprise a fusion effector protein, wherein the fusion effector protein comprises an effector protein described herein. In some embodiments, compositions comprise a nucleic acid encoding the fusion effector protein. In general, fusion effector proteins comprise an effector protein or a portion thereof, and a fusion partner protein. In some embodiments, compositions comprise a fusion effector protein and a guide nucleic acid, wherein at least a portion of the guide nucleic acid hybridizes to a target sequence of a target nucleic acid, and the fusion partner modulates the target nucleic acid or expression thereof. A fusion partner protein may also simply be referred to herein as a fusion partner. In general, the effector protein and the fusion partner protein are heterologous proteins. Alternatively, in some embodiments, the effector proteins described herein may function in combination with any of the fusion partner proteins described herein, wherein the effector protein and the fusion partner protein are not fused together.

In some cases, fusion effector proteins modify a target nucleic acid or the expression thereof. In some cases, the modifications are transient (e.g., transcription repression or activation). In some cases, the modifications are inheritable. For instance, epigenetic modifications made to a target nucleic acid, or to proteins associated with the target nucleic acid, e.g., nucleosomal histones, in a cell, are observed in cells produced by proliferation of the cell.

In some cases, fusion effector proteins modify a target nucleic acid or the expression thereof, wherein the target nucleic acid comprises a deoxyribonucleoside, a ribonucleoside or a combination thereof. The target nucleic acid may comprise or consist of a single stranded RNA (ssRNA), a double-stranded RNA (dsRNA), a single-stranded DNA (ssDNA), or a double stranded DNA (dsDNA). Non-limiting examples of fusion partners for modifying ssRNA include, but are not limited to, splicing factors (e.g., RS domains); protein translation components (e.g., translation initiation, elongation, and/or release factors; e.g., eIF4G); RNA methylases; RNA editing enzymes (e.g., RNA deaminases, e.g., adenosine deaminase acting on RNA (ADAR), including A to I and/or C to U editing enzymes); helicases; and RNA-binding proteins.

In some embodiments, the fusion partner protein is fused to the 5′ end of the effector protein. In some embodiments, the fusion partner protein is fused to the 3′ end of the effector protein. In some embodiments, the effector protein is located at an internal location of the fusion partner protein. In some embodiments, the fusion partner protein is located at an internal location of the Cas effector protein. For example, a base editing enzyme (e.g., a deaminase enzyme) is inserted at an internal location of a Cas effector protein. The effector protein may be fused directly or indirectly (e.g., via a linker) to the fusion partner protein. Exemplary linkers are described herein. Cas proteins may be fused to transcription activators or transcriptional repressors or deaminases or other nucleic acid modifying proteins. In some cases, Cas proteins need not be fused to a fusion partner protein to accomplish the required protein (expression) modification.

In some embodiments, an effector protein described herein comprises 1, 2, 3, 4, 5, 6, 7, 8, 9, 10, or more fusion partner proteins at or near the N-terminus, 1, 2, 3, 4, 5, 6, 7, 8, 9, 10, or more fusion partner proteins at or near the C-terminus, or a combination of these (e.g., one or more fusion partner proteins at the amino-terminus and one or more fusion partner proteins at the carboxy terminus). When more than one fusion partner protein is present, each may be selected independently of the others, such that a single fusion partner protein may be present in more than one copy and/or in combination with one or more other fusion partner proteins present in one or more copies. In some embodiments, a fusion partner protein is considered near the N- or C-terminus when the nearest amino acid of the fusion partner protein is within about 1, 2, 3, 4, 5, 10, 15, 20, 25, 30, 40, 50, or more amino acids along the polypeptide chain from the N- or C-terminus.

In some embodiments, a fusion partner may provide signaling activity. In some embodiments, a fusion partner may inhibit or promote the formation of multimeric complex of an effector protein. By way of non-limiting example, the fusion protein may comprise an effector protein described herein and a fusion partner comprising a Calcineurin A tag, wherein the fusion protein dimerizes in the presence of Tacrolimus (FK506). Also, by way of non-limiting example, the fusion protein may comprise an effector protein described herein and a SpyTag configured to dimerize or associate with another effector protein in a multimeric complex. In an additional example, the fusion partner may directly or indirectly edit a target nucleic acid. Edits can be of a nucleobase, nucleotide, or nucleotide sequence of a target nucleic acid. In some embodiments, the fusion partner may interact with additional proteins, or functional fragments thereof, to make modifications to a target nucleic acid. In other embodiments, the fusion partner may modify proteins associated with a target nucleic acid. In some embodiments, a fusion partner may modulate transcription (e.g., inhibits transcription, increases transcription) of a target nucleic acid. In yet another example, a fusion partner may directly or indirectly inhibit, reduce, activate or increase expression of a target nucleic acid.

### Modifying Target Nucleic Acid

In some cases, fusion partners provide enzymatic activity that modifies a target nucleic acid. Such enzymatic activities include, but are not limited to, nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, and glycosylase activity. In some cases, nuclease activity comprises the enzymatic activity of an enzyme which allows the enzyme to cleave the phosphodiester bonds between the nucleotide subunits of nucleic acids. In some case, an enzyme with nuclease activity can comprise a nuclease.

Disclosed herein are compositions and methods for modifying a target nucleic acid. The target nucleic acid may be a gene or a portion thereof. Methods and compositions may modify a coding portion of a gene, a non-coding portion of a gene, or a combination thereof. Modifying at least one gene using the compositions and methods described herein may reduce or increase expression of one or more genes. In some embodiments, compositions and methods reduce expression of one or more genes by at least 10%, at least 20%, at least 30%, at least 40%, at least 50%, at least 60%, at least 70%, at least 80%, at least 90%, or at least 95%. In some embodiments, compositions and methods remove all expression of a gene, also referred to as genetic knock out. In some embodiments, compositions and methods increase expression of one or more genes by at least 10%, at least 20%, at least 30%, at least 40%, at least 50%, at least 60%, at least 70%, at least 80%, at least 90%, or at least 100%.

In some embodiments, compositions and methods use effector proteins that are fused to a heterologous protein. Heterologous proteins include, but are not limited to, transcriptional activators, transcriptional repressors, deaminases, methyltransferases, acetyltransferases, and other nucleic acid modifying proteins. In some cases, effector proteins need not be fused to a partner protein to accomplish the required protein (expression) modification.

In some cases, fusion partners have enzymatic activity that modifies the target nucleic acid. The target nucleic acid may comprise or consist of a ssRNA, dsRNA, ssDNA, or a dsDNA. Examples of enzymatic activity that modifies the target nucleic acid include, but are not limited to: nuclease activity such as that provided by a restriction enzyme (e.g., FokI nuclease); methyltransferase activity such as that provided by a methyltransferase (e.g., HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants)); demethylase activity such as that provided by a demethylase (e.g., Ten-Eleven Translocation (TET) dioxygenase 1 (TET1CD), TET1, DME, DML1, DML2, ROS1); DNA repair activity; DNA damage (e.g., oxygenation) activity; deamination activity such as that provided by a deaminase (e.g., a cytosine deaminase enzyme such as rat APOBEC1); dismutase activity; alkylation activity; depurination activity; oxidation activity; pyrimidine dimer forming activity; integrase activity such as that provided by an integrase and/or resolvase (e.g., Gin invertase such as the hyperactive mutant of the Gin invertase, GinH106Y; human immunodeficiency virus type 1 integrase (IN); Tn3 resolvase); transposase activity, recombinase activity such as that provided by a recombinase (e.g., catalytic domain of Gin recombinase); as well as polymerase activity, ligase activity, helicase activity, photolyase activity, and glycosylase activity.

Non-limiting examples of fusion partners for targeting ssRNA include, but are not limited to, splicing factors (e.g., RS domains); protein translation components (e.g., translation initiation, elongation, and/or release factors; e.g., eIF4G); RNA methylases; RNA editing enzymes (e.g., RNA deaminases, e.g., adenosine deaminase acting on RNA (ADAR), including A to I and/or C to U editing enzymes); helicases; and RNA-binding proteins.

It is understood that a fusion protein may include the entire protein or in some cases may include a fragment of the protein (e.g., a functional domain). In some instances, the functional domain interacts with or binds ssRNA, including intramolecular and/or intermolecular secondary structures thereof, e.g., hairpins, stem-loops, etc.). In some instances, the functional domain interacts with or binds a nucleotide sequence, including intramolecular and/or intermolecular secondary structures thereof, e.g., hairpins, stem-loops, etc.). The functional domain may interact transiently or irreversibly, directly or indirectly. In some cases, a functional domain comprises a region of one or more amino acids in a protein that is required for an activity of the protein, or the full extent of that activity, as measured in an in vitro assay. Activities include, but are not limited to nucleic acid binding, nucleic acid modification, nucleic acid cleavage, protein binding. The absence of the functional domain, including mutations of the functional domain, would abolish or reduce activity.

Fusion proteins may comprise a protein or domain thereof selected from: endonucleases (e.g., RNase III, the CRR22 DYW domain, Dicer, and PIN (PilT N-terminus); SMG5 and SMG6; domains responsible for stimulating RNA cleavage (e.g., CPSF, CstF, CFIm and CFIIm); exonucleases such as XRN-1 or Exonuclease T; deadenylases such as HNT3; protein domains responsible for nonsense mediated RNA decay (e.g., UPF1, UPF2, UPF3, UPF3b, RNP S1, Y14, DEK, REF2, and SRm160); protein domains responsible for stabilizing RNA (e.g., PABP); proteins and protein domains responsible for repressing translation (e.g., Ago2 and Ago4); proteins and protein domains responsible for stimulating translation (e.g., Staufen); proteins and protein domains responsible for (e.g., capable of) modulating translation (e.g., translation factors such as initiation factors, elongation factors, release factors, etc., e.g., eIF4G); proteins and protein domains responsible for polyadenylation of RNA (e.g., PAP1, GLD-2, and Star-PAP); proteins and protein domains responsible for polyuridinylation of RNA (e.g., CI D1 and terminal uridylate transferase); proteins and protein domains responsible for RNA localization (e.g., from IMP1, ZBP1, She2p, She3p, and Bicaudal-D); proteins and protein domains responsible for nuclear retention of RNA (e.g., Rrp6); proteins and protein domains responsible for nuclear export of RNA (e.g., TAP, NXF1, THO, TREX, REF, and Aly); proteins and protein domains responsible for repression of RNA splicing (e.g., PTB, Sam68, and hnRNP A1); proteins and protein domains responsible for stimulation of RNA splicing (e.g., Serine/Arginine-rich (SR) domains); proteins and protein domains responsible for reducing the efficiency of transcription (e.g., FUS (TLS)); and proteins and protein domains responsible for stimulating transcription (e.g., CDK7 and HIV Tat). Alternatively, the effector domain may be a domain of a protein selected from the group comprising endonucleases; proteins and protein domains capable of stimulating RNA cleavage; exonucleases; deadenylases; proteins and protein domains having nonsense mediated RNA decay activity; proteins and protein domains capable of stabilizing RNA; proteins and protein domains capable of repressing translation; proteins and protein domains capable of stimulating translation; proteins and protein domains capable of modulating translation (e.g., translation factors such as initiation factors, elongation factors, release factors, etc., e.g., eIF4G); proteins and protein domains capable of polyadenylation of RNA; proteins and protein domains capable of polyuridinylation of RNA; proteins and protein domains having RNA localization activity; proteins and protein domains capable of nuclear retention of RNA; proteins and protein domains having RNA nuclear export activity; proteins and protein domains capable of repression of RNA splicing; proteins and protein domains capable of stimulation of RNA splicing; proteins and protein domains capable of reducing the efficiency of transcription; and proteins and protein domains capable of stimulating transcription. Another suitable fusion partner is a PUF RNA-binding domain, which is described in more detail in WO2012068627, which is hereby incorporated by reference in its entirety.

In some instances, the fusion partner comprises an RNA splicing factor. The RNA splicing factor may be used (in whole or as fragments thereof) for modular organization, with separate sequence-specific RNA binding modules and splicing effector domains. Non-limiting examples of RNA splicing factors include members of the Serine/Arginine-rich (SR) protein family contain N-terminal RNA recognition motifs (RRMs) that bind to exonic splicing enhancers (ESEs) in pre-mRNAs and C-terminal RS domains that promote exon inclusion. As another example, the hnRNP protein hnRNP A1 binds to exonic splicing silencers (ESSs) through its RRM domains and inhibits exon inclusion through a C-terminal Glycine-rich domain. Some splicing factors may regulate alternative use of splice site (ss) by binding to regulatory sequences between the two alternative sites. For example, ASF/SF2 may recognize ESEs and promote the use of intron proximal sites, whereas hnRNP A1 may bind to ESSs and shift splicing towards the use of intron distal sites. One application for such factors is to generate ESFs that modulate alternative splicing of endogenous genes, particularly disease associated genes. For example, Bcl-x pre-mRNA produces two splicing isoforms with two alternative 5′ splice sites to encode proteins of opposite functions. The long splicing isoform Bcl-xL is a potent apoptosis inhibitor expressed in long-lived postmitotic cells and is up-regulated in many cancer cells, protecting cells against apoptotic signals. The short isoform Bcl-xS is a pro-apoptotic isoform and expressed at high levels in cells with a high turnover rate (e.g., developing lymphocytes). The ratio of the two Bcl-x splicing isoforms is regulated by multiple c\acute{ω}-elements that are located in either the core exon region or the exon extension region (i.e., between the two alternative 5′ splice sites). For more examples, see WO2010075303, which is hereby incorporated by reference in its entirety.

### Base Editors

In some embodiments, fusion partners modify a nucleobase of a target nucleic acid. Fusion proteins comprising such fusion partners and an effector protein may be referred to as base editors. In some embodiments, base editors modify a sequence of a target nucleic acid. In some embodiments, a base editor comprises a base editing enzyme variant that differs from a naturally occurring base editing enzyme, but it is understood that any reference to a base editing enzyme herein also refers to a base editing enzyme variant. In some embodiments, base editors provide a nucleobase change in a DNA molecule. In some embodiments, the nucleobase change in the DNA molecule is selected from: an adenine (A) to guanine (G); cytosine (C) to thymine (T); and cytosine (C) to guanine (G). In some embodiments, base editors provide a nucleobase change in an RNA molecule. In some embodiments, the nucleobase change in the RNA molecule is selected from: adenine (A) to guanine (G); uracil (U) to cytosine (C); cytosine (C) to guanine (G); and guanine (G) to adenine (A). In some embodiments, the fusion partner is a deaminase, e.g., ADAR1/2.

Some base editors modify a nucleobase of on a single strand of DNA. In some embodiments, base editors modify a nucleobase on both strands of dsDNA. In some embodiments, upon binding to its target locus in DNA, base pairing between the guide RNA and target DNA strand leads to displacement of a small segment of single-stranded DNA in an “R-loop”. In some embodiments, DNA bases within the R-loop are modified by the deaminase enzyme. In some embodiments, DNA base editors for improved efficiency in eukaryotic cells comprise a catalytically inactive effector protein that may generate a nick in the non-edited DNA strand, inducing repair of the non-edited strand using the edited strand as a template.

In some embodiments, a catalytically inactive effector protein can comprise an effector protein that is modified relative to a naturally-occurring nuclease to have a reduced or eliminated catalytic activity relative to that of the naturally-occurring nuclease, but retains its ability to interact with a guide nucleic acid. The catalytic activity that is reduced or eliminated is often a nuclease activity. The naturally-occurring nuclease may be a wildtype protein. In some embodiments, the catalytically inactive effector protein is referred to as a catalytically inactive variant of a nuclease, e.g., a Cas nuclease.

Some base editors modify a nucleobase of an RNA. In some embodiments, RNA base editors comprise an adenosine deaminase. In some embodiments, ADAR proteins bind to RNAs and alter their sequence by changing an adenosine into an inosine. In some embodiments, RNA base editors comprise an effector protein that is activated by or binds RNA.

In some embodiments, base editors are used to treat a subject having or a subject suspected of having a disease related to a gene of interest. In some embodiments, base editors are useful for treating a disease or a disorder caused by a point mutation in a gene of interest. In some embodiments, compositions comprise a base editor and a guide nucleic acid, wherein the guide nucleic acid directs the base editor to a sequence in a target gene. The target gene may be associated with a disease. In some embodiments, the guide nucleic acid directs that base editor to or near a mutation in the sequence of a target gene. The mutation may be the deletion of one more nucleotides. The mutation may be the addition of one or more nucleotides. The mutation may be the substitution of one or more nucleotides. The mutation may be the insertion, deletion or substitution of a single nucleotide, also referred to as a point mutation. The point mutation may be a SNP. The mutation may be associated with a disease. In some embodiments, the guide nucleic acid directs the base editor to bind a target sequence within the target nucleic acid that is within 0, 1, 2, 3, 4, 5, 6, 7, 8, 9, 10, 11, 12, 13, 14, 15, 16, 17, 18, 19, or 20 nucleotides of the mutation. In some embodiments, the guide nucleic acid comprises a sequence that is identical, complementary or reverse complementary to a target sequence of a target nucleic acid that comprises the mutation. In some embodiments, the guide nucleic acid comprises a sequence that is identical, complementary or reverse complementary to a target sequence of a target nucleic acid that is within 1, 2, 3, 4, 5, 6, 7, 8, 9, 10, 11, 12, 13, 14, 15, 16, 17, 18, 19, or 20 nucleotides of the mutation.

Some base editors modify a nucleobase of an RNA. In some embodiments, RNA base editors comprise an adenosine deaminase. In some embodiments, ADAR proteins bind to RNAs and alter their sequence by changing an adenosine into an inosine. In some embodiments, RNA base editors comprise an effector protein that is activated by or binds RNA.

In some embodiments, base editors are used to treat a subject having or a subject suspected of having a disease related to a gene of interest. In some embodiments, base editors are useful for treating a disease or a disorder caused by a point mutation in a gene of interest. In some embodiments, compositions comprise a base editor and a guide nucleic acid, wherein the guide nucleic acid directs the base editor to a sequence in a target gene

In some embodiments, fusion partners comprise a base editing enzyme. In some embodiments, the base editing enzyme modifies the nucleobase of a deoxyribonucleotide. In some embodiments, the base editing enzyme modifies the nucleobase of a ribonucleotide. A base editing enzyme that converts a cytosine to a guanine or thymine may be referred to as a cytosine base editing enzyme. A base editing enzyme that converts an adenine to a to a guanine may be referred to as an adenine base editing enzyme. In some embodiments, the base editing enzyme comprises a deaminase enzyme. In some embodiments, the deaminase functions as a monomer. In some embodiments, the deaminase functions as heterodimer with an additional protein. In some embodiments, base editors comprise a DNA glycosylase inhibitor. In some embodiments, base editors comprise a uracil glycosylase inhibitor (UGI) or uracil N-glycosylase (UNG). In some embodiments, base editors do not comprise a UGI. In some embodiments, base editors do not comprise a UNG. In some embodiments, base editors do not comprise a functional fragment of a UGI. A functional fragment of a UGI is a fragment of a UGI that is capable of excising a uracil residue from DNA by cleaving an N-glycosydic bond. In some cases, a functional fragment comprises a fragment of a protein that retains some function relative to the entire protein. Non-limiting examples of functions are nucleic acid binding, protein binding, nuclease activity, nickase activity, deaminase activity, demethylase activity, or acetylation activity.

In some embodiments, a base editing enzyme can comprise a protein, polypeptide or fragment thereof that is capable of catalyzing the chemical modification of a nucleobase of a deoxyribonucleotide or a ribonucleotide. Such a base editing enzyme, for example, is capable of catalyzing a reaction that modifies a nucleobase that is present in a nucleic acid molecule, such as DNA or RNA (single stranded or double stranded). Non-limiting examples of the type of modification that a base editing enzyme is capable of catalyzing includes converting an existing nucleobase to a different nucleobase, such as converting a cytosine to a guanine or thymine or converting an adenine to a guanine, hydrolytic deamination of an adenine or adenosine, or methylation of cytosine (e.g., CpG, CpA, CpT or CpC). A base editing enzyme itself may or may not bind to the nucleic acid molecule containing the nucleobase. In some cases, a base editor can be a fusion protein comprising a base editing enzyme fused to an effector protein. The base editor is functional when the effector protein is coupled to a guide nucleic acid. The guide nucleic acid imparts sequence specific activity to the base editor. By way of non-limiting example, the effector protein may comprise a catalytically inactive effector protein. Also, by way of non-limiting example, the base editing enzyme may comprise deaminase activity. Additional base editors are described herein.

In some embodiments, the base editor is a cytidine deaminase base editor generated by ancestral sequence reconstruction as described in WO2019226953, which is hereby incorporated by reference in its entirety.

Exemplary deaminase domains are described WO 2018027078 and WO2017070632, and each are hereby incorporated in its entirety by reference. Also, additional exemplary deaminase domains are described in Komor et al., Nature, 533, 420-424 (2016); Gaudelli et al., Nature, 551, 464-471 (2017); Komor et al., Science Advances, 3:eaao4774 (2017), and Rees et al., Nat Rev Genet. 2018 December; 19(12):770-788. doi: 10.1038/s41576-018-0059-1, which are hereby incorporated by reference in their entirety.

In some embodiments, the base editor is a cytosine base editor (CBE). In general, a CBE comprises a cytosine base editing enzyme and a catalytically inactive effector protein. In some embodiments, the catalytically inactive effector protein is a catalytically inactive variant of an effector protein described herein. The CBE may convert a cytosine to a thymine. In some embodiments, the base editor is an adenine base editor (ABE). In general, an ABE comprises an adenine base editing enzyme and a catalytically inactive effector protein. In some embodiments, the catalytically inactive effector protein is a catalytically inactive variant of an effector protein described herein. The ABE generally converts an adenine to a guanine. In some embodiments, the base editor is a cytosine to guanine base editor (CGBE). In general, a CGBE converts a cytosine to a guanine.

In some embodiments, the base editor is a CBE. In some embodiments, the cytosine base editing enzyme is a cytidine deaminase. In some embodiments, the cytosine deaminase is an APOBEC1 cytosine deaminase, which accept ssDNA as a substrate but is incapable of cleaving dsDNA, fused to a catalytically inactive effector protein. In some embodiments, when bound to its cognate DNA, the catalytically inactive effector protein performs local denaturation of the DNA duplex to generate an R-loop in which the DNA strand not paired with the guide RNA exists as a disordered single-stranded bubble. In some embodiments, the catalytically inactive effector protein generated ssDNA R-loop enables the CBE to perform efficient and localized cytosine deamination in vitro. In some examples, deamination activity is exhibited in a window of about 4 to about 10 base pairs. In some embodiments, fusion to the catalytically inactive effector protein presents the target site to APOBEC1 in high effective molarity, enabling the CBE to deaminate cytosines located in a variety of different sequence motifs, with differing efficacies. In some embodiments, the CBE is capable of mediating RNA-programmed deamination of target cytosines in vitro. In some embodiments, the CBE is capable of mediating RNA-programmed deamination of target cytosines in vivo. In some embodiments, the cytosine base editing enzyme is a cytosine base editing enzyme described by Koblan et al. (2018) Nature Biotechnology 36:848-846; Komor et al. (2016) Nature 533:420-424; Koblan et al. (2021) “Efficient C•G-to-G•C base editors developed using CRISPRi screens, target-library analysis, and machine learning,” Nature Biotechnology; Kurt et al. (2021) Nature Biotechnology 39:41-46; Zhao et al. (2021) Nature Biotechnology 39:35-40; and Chen et al. (2021) Nature Communications 12:1384, all incorporated herein by reference.

In some embodiments, CBEs comprise a uracil glycosylase inhibitor (UGI) or uracil N-glycosylase (UNG). In some embodiments, base excision repair (BER) of U•G in DNA is initiated by a UNG, which recognizes the U•G mismatch and cleaves the glyosidic bond between uracil and the deoxyribose backbone of DNA. In some embodiments, BER results in the reversion of the U•G intermediate created by the first CBE back to a C•G base pair. In some embodiments, UNG may be inhibited by fusion of uracil DNA glycosylase inhibitor (UGI), in some embodiments, a small protein from bacteriophage PBS, to the C-terminus of the CBE. In some embodiments, UGI is a DNA mimic that potently inhibits both human and bacterial UNG. In some embodiments, a UGI inhibitor is any protein or polypeptide that inhibits UNG. In some embodiments, the CBE mediates efficient base editing in bacterial cells and moderately efficient editing in mammalian cells, enabling conversion of a C•G base pair to a T•A base pair through a U•G intermediate. In some embodiments, the CBE is modified to increase base editing efficiency while editing more than one strand of DNA.

In some embodiments, the CBE nicks the non-edited DNA strand. In some embodiments, the non-edited DNA strand nicked by the CBE biases cellular repair of the U•G mismatch to favor a U•A outcome, elevating base editing efficiency. In some embodiments, the APOBEC1-nickase-UGI fusion efficiently edits in mammalian cells, while minimizing frequency of non-target indels. In some embodiments, base editors do not comprise a functional fragment of the base editing enzyme. In some embodiments, base editors do not comprise a function fragment of a UGI, where such a fragment may be capable of excising a uracil residue from DNA by cleaving an N-glycosidic bond.

In some embodiments, the cytidine deaminase is selected from APOBEC1, APOBEC2, APOBEC3C, APOBEC3D, APOBEC3F, APOBEC3G, APOBEC3H, APOBEC4, APOBEC3A, BE1 (APOBEC1-XTEN-dCas9), BE2 (APOBEC1-XTEN-dCas9-UGI), BE3 (APOBEC1-XTEN-dCas9(A840H)-UGI), BE3-Gam, saBE3, saBE4-Gam, BE4, BE4-Gam, saBE4, or saBE4-Gam as described in WO2021163587, WO202108746, WO2021062227, and WO2020123887, which are incorporated herein by reference in their entirety.

In some embodiments, the fusion protein further comprises a non-protein uracil-DNA glcosylase inhibitor (npUGI). In some embodiments, the npUGI is selected from a group of small molecule inhibitors of uracil-DNA glycosylase (UDG), or a nucleic acid inhibitor of UDG. In some embodiments, the non-protein uracil-DNA glcosylase inhibitor (npUGI) is a small molecule derived from uracil. Examples of small molecule non-protein uracil-DNA glcosylase inhibitors, fusion proteins, and Cas-CRISPR systems comprising base editing activity are described in WO202108746, which is incorporated by reference in its entirety.

In some embodiments, the fusion partner is a deaminase, e.g., ADAR1/2, ADAR-2, or AID. In some embodiments, the base editor is an ABE. In some embodiments, the adenine base editing enzyme of the ABE is an adenosine deaminase. In some embodiments, the adenine base editing enzyme is selected from ABE8e, ABE8.20m, APOBEC3A, Anc APOBEC (a.k.a. AncBE4Max), and BtAPOBEC2. In some embodiments, the ABE base editor is an ABE7 base editor. In some embodiments, the deaminase or enzyme with deaminase activity is selected from ABE8.1m, ABE8.2m, ABE8.3m, ABE8.4m, ABE8.5m, ABE8.6m, ABE8.7m, ABE8.8m, ABE8.9m, ABE8.10m, ABE8.11m, ABE8.12m, ABE8.13m, ABE8.14m, ABE8.15m, ABE8.16m, ABE8.17m, ABE8.18m, ABE8.19m, ABE8.20m, ABE8.21m, ABE8.22m, ABE8.23m, ABE8.24m, ABE8.1d, ABE8.2d, ABE8.3d, ABE8.4d, ABE8.5d, ABE8.6d, ABE8.7d, ABE8.8d, ABE8.9d, ABE8.10d, ABE8.11d, ABE8.12d, ABE8.13d, ABE8.14d, ABE8.15d, ABE8.16d, ABE8.17d, ABE8.18d, ABE8.19d, ABE8.20d, ABE8.21d, ABE8.22d, ABE8.23d, or ABE8.24d. In some embodiments, the adenine base editing enzyme is ABE8.1d. In some embodiments, the adenosine base editor is ABE9. Exemplary deaminases are described in US20210198330, WO2021041945, WO2021050571A1, and WO2020123887, all of which are incorporated herein by reference in their entirety. In some embodiments, the adenine base editing enzyme is an adenine base editing enzyme described in Chu et al., (2021) The CRISPR Journal 4:2:169-177, incorporated herein by reference. In some embodiments, the adenine deaminase is an adenine deaminase described by Koblan et al. (2018) Nature Biotechnology 36:848-846, incorporated herein by reference. In some embodiments, the adenine base editing enzyme is an adenine base editing enzyme described by Tran et al. (2020) Nature Communications 11:4871. Additional examples of deaminase domains are also described in WO2018027078 and WO2017070632, which are hereby incorporated by reference in their entirety.

In some embodiments, an ABE converts an A•T base pair to a G•C base pair. In some embodiments, the ABE converts a target A•T base pair to G•C in vivo. In some embodiments, the ABE converts a target A•T base pair to G•C in vitro. In some embodiments, ABEs provided herein reverse spontaneous cytosine deamination, which has been linked to pathogenic point mutations. In some embodiments, ABEs provided herein enable correction of pathogenic SNPs (˜47% of disease-associated point mutations). In some embodiments, the adenine comprises exocyclic amine that has been deaminated (e.g., resulting in altering its base pairing preferences). In some embodiments, deamination of adenosine yields inosine. In some embodiments, inosine exhibits the base-pairing preference of guanine in the context of a polymerase active site, although inosine in the third position of a tRNA anticodon is capable of pairing with A, U, or C in mRNA during translation. In some embodiments, an ABE comprises an engineered adenosine deaminase enzyme capable of acting on ssDNA.

In some embodiments, abase editor comprises an adenosine deaminase variant that differs from a naturally occurring deaminase. Relative to the naturally occurring deaminase, the adenosine deaminase variant may comprise a V82S alteration, a T166R alteration, or a combination thereof. In some embodiments, the adenosine deaminase variant comprises at least one of the following alterations relative to a naturally occurring adenosine deaminase: Y147T, Y147R, Q154S, Y123H, and Q154R., which are incorporated herein by reference in their entirety.

In some embodiments, a base editor comprises a deaminase dimer. In some embodiments, a base editor is a deaminase dimer further comprising a base editing enzyme and an adenine deaminase (e.g., TadA).

TadA comprises or consists of at least a portion of the sequence: SEVEFSHEYWMRHALTLAKRAWDEREVPVGAVLVHNNRVIGEGWNRPIGRHDPTAHAEIM ALRQGGLVMQNYRLIDATLYVTLEPCVMCAGAMIHSRIGRVVFGARDAKTGAAGSLMDVL HHPGMNHRVEITEGILADECAALLSDFFRMRRQEIKAQKKAQSSTD (SEQ ID NO: 426).

In some embodiments, the adenosine deaminase is a TadA monomer (e.g., Tad*7.10, TadA*8 or TadA*9). In some embodiments, the adenosine deaminase is a TadA*8 variant. Such a TadA*8 variant includes TadA*8.1, TadA*8.2, TadA*8.3, TadA*8.4, TadA*8.5, TadA*8.6, TadA*8.7, TadA*8.8, TadA*8.9, TadA*8.10, TadA*8.11, TadA*8.12, TadA*8.13, TadA*8.14, TadA*8.15, TadA*8.16, TadA*8.17, TadA*8.18, TadA*8.19, TadA*8.20, TadA*8.21, TadA*8.22, TadA*8.23, or TadA*8.24 as described in WO2021163587 and WO2021050571, which are each hereby incorporated by reference in its entirety.

In some embodiments, a base editor is a deaminase dimer comprising a base editing enzyme fused to TadA via a linker. In some embodiments the linker comprises or consists of at least a portion of the sequence: SGGSSGGSSGSETPGTSESATPESSGGSSGGS (SEQ ID NO: 427). In some embodiments, the amino acid sequence of the linker is 70%, 75%, 80%, 85%, 90%, or 95% identical to SGGSSGGSSGSETPGTSESATPESSGGSSGGS (SEQ ID NO: 427).

In some embodiments, the amino terminus of the fusion partner protein is linked to the carboxy terminus of the effector protein via the linker. In some embodiments, the carboxy terminus of the fusion partner protein is linked to the amino terminus of the effector protein via the linker.

In some embodiments, the base editing enzyme is fused to TadA at the N-terminus. In some embodiments, the base editing enzyme is fused to TadA at the C-terminus. In some embodiments, the base editing enzyme is a deaminase dimer comprising an ABE. In some embodiments, the deaminase dimer comprises an adenosine deaminase. In some embodiments, the deaminase dimer comprises TadA fused to an adenine base editing enzyme selected from ABE8e, ABE8.20m, APOBEC3A, Anc APOBEC (a.k.a. AncBE4Max), and BtAPOBEC2. In some embodiments TadA is fused to ABE8e or a variant thereof. In some embodiments TadA is fused to ABE8e or a variant thereof at the amino-terminus (ABE8e-TadA). In some embodiments, TadA is fused to ABE8e or a variant thereof at the carboxy terminus (ABE8e-TadA).

### Prime Editing

In some embodiments, a fusion protein and/or a fusion partner can comprise a prime editing enzyme. In some embodiments, a fusion protein comprises an effector protein and a prime editing enzyme. In some embodiments, the effector protein is a nickase. In some embodiments, the effector protein is capable of breaking a single strand of a double stranded DNA molecule. In some embodiments, a prime editing enzyme is a protein, a polypeptide or a fragment thereof that is capable of catalyzing a reaction for modifying a target nucleic acid. In some embodiments, a modified target nucleic acid comprises one or more insertions, deletions, or base-to-base conversions relative to the target nucleic acid before modification. Accordingly, in some embodiments, a prime editing enzyme is a protein, a polypeptide or a fragment thereof that is capable of catalyzing the modification (insertion, deletion, or base-to-base conversion) of a target nucleotide or nucleotide sequence in a nucleic acid. In some embodiments, a prime editing enzyme capable of catalyzing such a reaction includes a reverse transcriptase. In some embodiments, such a prime editing enzyme is an RNA dependent DNA polymerase (RDDP). (e.g., Moloney Murine Leukemia Virus reverse transcriptase (M-MLV RT) or a mutant thereof). Accordingly, in some embodiments, such a prime editing enzyme is an M-MLV RT enzyme or a mutant thereof. In some embodiments, the M-MLV RT enzyme comprises at least one mutation selected from D200N, L603W, T330P, T306K, and W313F relative to wildtype M-MLV RT enzyme.

A prime editing enzyme may require a prime editing guide RNA (pegRNA) to catalyze the modification. A pegRNA generally comprises a primer binding site and a template RNA comprising the modification. The primer binding site and template RNA are linked to the guide RNA, generally at the 3′ end of the guide RNA. Such a pegRNA can be capable of identifying the target sequence to be edited and encoding the new genetic information that replaces the targeted nucleotide or nucleotide sequence in the nucleic acid. A prime editing enzyme may require a prime editing guide RNA (pegRNA) and a single guide RNA to catalyze the modification. In some embodiments, a prime editing guide RNA (pegRNA) comprises an RNA aptamer. In some embodiments, the RNA aptamer recruits other protein (e.g., MS2 coat protein).

In some embodiments, the effector protein is fused to the prime editing enzyme. In some embodiments, the effector protein is not fused to the prime editing enzyme. In some embodiments, the prime editing enzyme is fused to a protein that binds an RNA aptamer, wherein the pegRNA comprises the RNA aptamer, thereby recruiting an MS2 coat protein to the target nucleic acid. In some embodiments, the effector protein comprises an amino acid sequence that is at least 90%, at least 95%, at least 98%, at least 99% or 100% identical to SEQ ID NO: 499.

### Recombinases

In some embodiments, the fusion partners comprise a recombinase domain. In some embodiments, the recombinase is a site-specific recombinase. Examples of site-specific recombinases include a tyrosine recombinase, a serine recombinase, an integrase, or mutants or variants thereof. In some embodiments, the site-specific recombinase is a tyrosine recombinase. Non-limiting examples of a tyrosine recombinase is Cre, Flp or lambda integrase. In some embodiments, the recombinase is a serine recombinase. Non-limiting examples of serine recombinases include, but are not limited to, gamma-delta resolvase, Tn3 resolvase, Sin resolvase, Gin invertase, Hin invertase, Tn5044 resolvase, IS607 transposase, and IS607 integrase. In some embodiments, the site-specific recombinase is an integrase. Non-limiting examples of integrases include, but are not limited to: Bxb1, wBeta, BL3, phiR4, A118, TG1, MR11, phi370, SPBc, TP901-1, phiRV, FC1, K38, phiBT1, and phiC31. In some embodiments, the recombinase is a Tn5 transposase, SB100X, phage encoded serine integrases/recombinase 2, phage encoded serine integrase/recombinase 13, or Human WT Exonuclease 1a. Further discussion and examples of suitable recombinase fusion partners are described in U.S. Pat. No. 10,975,392, which is incorporated herein by reference in its entirety.

In some embodiments, the fusion protein comprises a linker that links the recombinase domain to the Cas-CRISPR domain of the effector protein. In some embodiments, the linker is The-Ser.

### Modifying Proteins

In some cases, a fusion partner provides enzymatic activity that modifies a protein (e.g., a histone) associated with a target nucleic acid. Such enzymatic activities include, but are not limited to, methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, de-ribosylation activity, myristoylation activity, and demyristoylation activity. In some embodiments, the fusion partner provides enzymatic activities that may include, but are not limited to, nuclease activity, DNA repair activity, DNA damage activity, deamination activity, dismutase activity, alkylation activity, depurination activity, oxidation activity, dimer forming activity (e.g., pyrimidine dimer forming activity), integrase activity, transposase activity, recombinase activity, polymerase activity, ligase activity, helicase activity, photolyase activity, and glycosylase activity.

In some cases, the fusion partner has enzymatic activity that modifies a protein associated with a target nucleic acid. The protein may be a histone, an RNA binding protein, or a DNA binding protein. Examples of such protein modification activities include methyltransferase activity such as that provided by a histone methyltransferase (HMT) (e.g., suppressor of variegation 3-9 homolog 1 (SUV39H1, also known as KMT1A), euchromatic histone lysine methyltransferase 2 (G9A, also known as KMT1C and EHMT2), SUV39H2, ESET/SETDB1, SET1A, SET1B, MLL1 to 5, ASH1, SYMD2, NSD1, DOT1L, Pr-SET7/8, SUV4-20H1, EZH2, RIZ1); demethylase activity such as that provided by a histone demethylase (e.g., Lysine Demethylase 1A (KDM1A also known as LSD1), JHDM2a/b, JMJD2A/JHDM3A, JMJD2B, JMJD2C/GASC1, JMJD2D, JARID1A/RBP2, JARID1B/PLU-1, JARID1C/SMCX, JARID1D/SMCY, UTX, JMJD3); acetyltransferase activity such as that provided by a histone acetylase transferase (e.g., catalytic core/fragment of the human acetyltransferase p300, GCN5, PCAF, CBP, TAF1, TIP60/PLIP, MOZ/MYST3, MORF/MYST4, HBO1/MYST2, HMOF/MYST1, SRC1, ACTR, P160, CLOCK); deacetylase activity such as that provided by a histone deacetylase (e.g., HDAC1, HDAC2, HDAC3, HDAC8, HDAC4, HDAC5, HDAC7, HDAC9, SIRT1, SIRT2, HDAC11); kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, and demyristoylation activity.

### CRISPRi Fusions

In some embodiments, fusions partners inhibit or reduce expression of a target nucleic acid. Fusion proteins comprising such fusion partners and an effector protein may be referred to as CRISPRi fusions. In some embodiments, fusion partners reduce expression of the target nucleic acid relative to its expression in the absence of the fusion effector protein. Relative expression, including transcription and RNA levels, may be assessed, quantified, and compared, e.g., by RT-qPCR. In some embodiments, fusion partners may comprise a transcriptional repressor. Transcriptional repressors may inhibit transcription via: recruitment of other transcription factor proteins; modification of target DNA such as methylation; recruitment of a DNA modifier; modulation of histones associated with target DNA; recruitment of a histone modifier such as those that modify acetylation and/or methylation of histones; or a combination thereof.

Non-limiting examples of fusion partners that decrease or inhibit transcription include, but are not limited to: transcriptional repressors such as the Kruppel associated box (KRAB or SKD); KOX1 repression domain; the ZIM3 KRAB domain, the Mad mSIN3 interaction domain (SID); the ERF repressor domain (ERD), the SRDX repression domain (e.g., for repression in plants); histone lysine methyltransferases such as Pr-SET7/8, SUV4-20H1, RIZ1, and the like; histone lysine demethylases such as JMJD2A/JHDM3A, JMJD2B, JMJD2C/GASC1, JMJD2D, JARID1A/RBP2, JARID1B/PLU-1, JARID1C/SMCX, JARID1D/SMCY; histone lysine deacetylases such as HDAC1, HDAC2, HDAC3, HDAC8, HDAC4, HDAC5, HDAC7, HDAC9, SIRT1, SIRT2, HDAC11; DNA methylases such as HhaI DNA m5c-methyltransferase (M.HhaI), DNA methyltransferase 1 (DNMT1), DNA methyltransferase 3a (DNMT3a), DNA methyltransferase 3b (DNMT3b), DNA methyltransferase 3 like (DNMT3L), METI, DRM3 (plants), ZMET2, CMT1, CMT2 (plants); and periphery recruitment elements such as Lamin A, and Lamin B; and functional domains thereof.

### CRISPRa Fusions

In some embodiments, fusion partners activate or increase expression of a target nucleic acid. Fusion proteins comprising such fusion partners and an effector protein may be referred to as CRISPRa fusions. In some embodiments, fusion partners increase expression of the target nucleic acid relative to its expression in the absence of the fusion effector protein. Relative expression, including transcription and RNA levels, may be assessed, quantified, and compared, e.g., by RT-qPCR. In some embodiments, fusion partners comprise a transcriptional activator. Transcriptional activators may promote transcription via: recruitment of other transcription factor proteins; modification of target DNA such as demethylation; recruitment of a DNA modifier; modulation of histones associated with target DNA; recruitment of a histone modifier such as those that modify acetylation and/or methylation of histones; or a combination thereof. Non-limiting examples of fusion partners that promote or increase transcription include, but are not limited to: transcriptional activators such as VPR, VP16, VP64, VP48, VP160, p65 subdomain (e.g., from NFkB), and activation domain of EDLL and/or TAL activation domain (e.g., for activity in plants); histone lysine methyltransferases such as SET1A, SET1B, MLL1 to 5, ASH1, SYMD2, NSD1; histone lysine demethylases such as JHDM2a/b, UTX, JMJD3; histone acetyltransferases such as GCN5, PCAF, CBP, p300, TAF1, TIP60/PLIP, MOZ/MYST3, MORF/MYST4, SRC1, ACTR, P160, CLOCK; and DNA demethylases such as Ten-Eleven Translocation (TET) dioxygenase 1 (TET1CD), TET1, TET2, DME, DML1, DML2, and ROS1; and functional domains thereof.

### Additional Fusion Partners

An effector protein disclosed herein or fusion effector protein may comprise a nuclear localization signal (NLS). In some embodiments, the NLS facilitates localization of a nucleic acid, protein, or small molecule to the nucleus, when present in a cell that contains a nuclear compartment. TABLE 1.1 lists exemplary NLS sequences. In some embodiments, a heterologous polypeptide described herein comprises a heterologous polypeptide sequence recited in TABLE 1.1.

In some embodiments, effector proteins described herein comprise an amino acid sequence that is at least about 65%, at least about 70%, at least about 75%, at least about 80%, at least about 85%, at least about 90%, at least about 95%, at least about 97%, at least about 98%, at least about 99%, or about 100% identical to any one of the sequences recited in TABLE 1 and further comprises one or more of the sequences set forth in TABLE 1.1. In some cases, the NLS may comprise a sequence of KRPAATKKAGQAKKKK (SEQ ID NO: 433). In some cases, the NLS comprises or consists of a sequence of PKKKRKV (SEQ ID NO: 434). In some cases, the NLS comprises or consists of a sequence of LPPLERLTL (SEQ ID NO: 435). The NLS may be located at a variety of locations, including, but not limited to 5′ of the effector protein, 5′ of the fusion partner, 3′ of the effector protein, 3′ of the fusion partner, between the effector protein and the fusion partner, within the fusion partner, within the effector protein.

An effector protein disclosed herein or fusion effector protein may comprise a nuclear export signal (NES), a mitochondrial localization signal, a chloroplast localization signal, an ER retention signal or combination thereof. In some embodiments, the NES retains the fusion effector protein in the cytoplasm. In some embodiments, the mitochondrial localization signal targets the fusion protein to the mitochondria. In some embodiments, the chloroplast localization signal targets the fusion protein to a chloroplast.

In some cases, the fusion partner is a chloroplast transit peptide (CTP), also referred to as a plastid transit peptide. In some instances, this targets the fusion protein to a chloroplast. Chromosomal transgenes from bacterial sources must have a sequence encoding a CTP sequence fused to a sequence encoding an expressed protein if the expressed protein is to be compartmentalized in the plant plastid (e.g. chloroplast). The CTP is removed in a processing step during translocation into the plastid. Accordingly, localization of an exogenous protein to a chloroplast is often accomplished by means of operably linking a polynucleotide sequence encoding a CTP sequence to the 5′ region of a polynucleotide encoding the exogenous protein. In some cases, the CTP is located at the N-terminus of the fusion protein. Processing efficiency may, however, be affected by the amino acid sequence of the CTP and nearby sequences at the amino terminus (NH2 terminus) of the peptide.

In some cases, the fusion partner is an endosomal escape peptide. An endosomal escape peptide is an agent that quickly disrupts the endosome in order to minimize the amount of time that a delivered molecule, such an effector protein, spends in the endosome-like environment, and to avoid getting trapped in the endosomal vesicles and degraded in the lysosomal compartment. In some cases, an endosomal escape protein comprises the amino acid sequence GLFXALLXLLXSLWXLLLXA (SEQ ID NO: 428), wherein each X is independently selected from lysine, histidine, and arginine. In some cases, an endosomal escape protein comprises the amino acid sequence GLFHALLHLLHSLWHLLLHA (SEQ ID NO: 429). In some cases, the amino acid sequence of the endosomal escape protein is SEQ ID NO: 428 or SEQ ID NO: 429.

Further suitable fusion partners include, but are not limited to, proteins (or fragments/domains thereof) that are boundary elements (e.g., CTCF), proteins and fragments thereof that provide periphery recruitment (e.g., Lamin A, Lamin B, etc.), protein docking elements (e.g., FKBP/FRB, P111/Aby1, etc.).

An effector protein disclosed herein or fusion effector protein comprises an endosomal escape peptide (EEP).

Further suitable fusion partners include a cell penetrating peptide (CPP), also known as a Protein Transduction Domain (PTD). A CPP or PTD is a polypeptide, polynucleotide, carbohydrate, or organic or inorganic compound that facilitates traversing a lipid bilayer, micelle, cell membrane, organelle membrane, or vesicle membrane.

### Linkers for Fusion Partners

In general, effector proteins and fusion partners of a fusion effector protein are connected via a linker. The linker may comprise or consist of a covalent bond. The linker may comprise or consist of a chemical group. In some embodiments, the linker comprises an amino acid. In some cases, a linker comprises a bond or molecule that links a first polypeptide to a second polypeptide. In some instances, a peptide linker comprises at least two amino acids linked by an amide bond. In general, the linker connects a terminus of the effector protein to a terminus of the fusion partner. In some embodiments, the carboxy terminus of the effector protein is linked to the amino terminus of the fusion partner. In some embodiments, the carboxy terminus of the fusion partner is linked to the amino terminus of the effector protein.

In some cases, a terminus of the effector protein is linked to a terminus of the fusion partner through an amide bond. In some cases, an effector protein is coupled to a fusion partner via a linker protein. The linker protein may have any of a variety of amino acid sequences. A linker protein may comprise a region of rigidity (e.g., beta sheet, alpha helix), a region of flexibility, or any combination thereof. In some embodiments, the linker comprises small amino acids, such as glycine and alanine, that impart linker flexibility. In some embodiments, the linker comprises amino acids that impart linker rigidity, such as valine and isoleucine. In some instances, the linker comprises small amino acids, such as glycine and alanine, that impart high degrees of flexibility. The ordinarily skilled artisan will recognize that design of a peptide conjugated to any desired element may include linkers that are all or partially flexible, such that the linker may include a flexible linker as well as one or more portions that confer less flexible structure. Suitable linkers include proteins of 4 linked amino acids to 40 linked amino acids in length, or between 4 linked amino acids and 25 linked amino acids in length. In some cases, a linked amino acid comprises at least two amino acids linked by an amide bond. These linkers may be produced by using synthetic, linker-encoding oligonucleotides to couple the proteins, or may be encoded by a nucleic acid sequence encoding a fusion protein (e.g., an effector protein coupled to a fusion partner). Examples of linker proteins include glycine polymers (G)n, glycine-serine polymers (including, for example, (GS)n, GSGGSn, GGSGGSn, and GGGSn, where n is an integer of at least one), glycine-alanine polymers, and alanine-serine polymers. Exemplary linkers may comprise amino acid sequences including, but not limited to, GGSG, GGSGG, GSGSG, GSGGG, GGGSG, and GSSSG.

In some embodiments, linkers comprise or consist of 4 to 60, 6 to 55, 8 to 50, 10 to 45, 12 to 40, 14 to 35, 16 to 30, 18 to 25 linked amino acids. In some embodiments, linkers comprise or consist of 1 to 10, 10 to 20, 20 to 30, 30 to 40, 40 to 50, or 50 to 60 linked amino acids. In some embodiments, linkers comprise or consist of 5 to 10, 10 to 15, 15 to 20, 20 to 25, 25 to 30, 30 to 35, 35 to 40, 40 to 45, 45 to 50, 50 to 55 or 55 to 60 linked amino acids. In some embodiments, linkers comprise or consist of about 5, about 10, about 15, about 20, about 25, about 30, about 35, about 40, about 45, about 50, about 55 or about 60 amino acids.

In some embodiments, linkers comprise or consists of a non-peptide linker. Non-limiting examples of non-peptide linkers are linkers comprising polyethylene glycol (PEG), polypropylene glycol (PPG), co-poly(ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacrylamide, polyacrylate, polycyanoacrylates, lipid polymers, chitins, hyaluronic acid, heparin, an alkyl linker, or a combination thereof.

In some embodiments, linkers comprise or consist of a nucleic acid. In some embodiments, the nucleic acid comprises DNA. In some embodiments, the nucleic acid comprises RNA. In some embodiments, the effector protein and the fusion partner each interact with the nucleic acid, the nucleic acid thereby linking the effector protein and the fusion partner. In some embodiments, the nucleic acid serves as a scaffold for both the effector protein and the fusion partner to interact with, thereby linking the effector protein and the fusion partner. Such nucleic acids include those described by Tadakuma et al., (2016), Progress in Molecular Biology and Translational Science, Volume 139, 2016, Pages 121-163, incorporated herein by reference.

In some embodiments, the fusion effector protein or the guide nucleic acid comprises a chemical modification that allows for direct crosslinking between the guide nucleic acid or the effector protein and the fusion partner. By way of non-limiting example, the chemical modification may comprise any one of a SNAP-tag, CLIP-tag, ACP-tag, Halo-tag, and an MCP-tag. In some embodiments, modifications are introduced with a Click Reaction, also known as Click Chemistry. The Click reaction may be copper dependent or copper independent.

In some embodiments, guide nucleic acids comprise an aptamer. The aptamer may serve as a linker between the effector protein and the fusion partner by interacting non-covalently with both. In some embodiments, the aptamer binds a fusion partner, wherein the fusion partner is a transcriptional activator. In some embodiments, the aptamer binds a fusion partner, wherein the fusion partner is a transcriptional inhibitor. In some embodiments, the aptamer binds a fusion partner, wherein the fusion partner comprises a base editor. In some embodiments, the aptamer binds the fusion partner directly. In some embodiments, the aptamer binds the fusion partner indirectly. Aptamers may bind the fusion partner indirectly through an aptamer binding protein. By way of non-limiting example, the aptamer binding protein may be MS2 and the aptamer sequence may be ACATGAGGATCACCCATGT (SEQ ID NO: 430); the aptamer binding protein may be PP7 and the aptamer sequence may be GGAGCAGACGATATGGCGTCGCTCC (SEQ ID NO: 431); or the aptamer binding protein may be BoxB and the aptamer sequence may be GCCCTGAAGAAGGGC (SEQ ID NO: 432).

In some embodiments, the fusion partner is located within effector protein. For example, the fusion partner may be a domain of a fusion partner protein that is internally integrated into the effector protein. In other words, the fusion partner may be located between the 5′ and 3′ ends of the effector protein without disrupting the ability of the fusion effector protein to recognize/bind a target nucleic acid. In some embodiments, the fusion partner replaces a portion of the effector protein. In some embodiments, the fusion partner replaces a domain of the effector protein. In some embodiments, the fusion partner does not replace a portion of the effector protein.

Effector proteins of the present disclosure may be produced in vitro or by eukaryotic cells or by prokaryotic cells. Effector proteins can be further processed by unfolding, e.g., heat denaturation, dithiothreitol reduction, etc. and may be further refolded, using any suitable method. Effector proteins of the present disclosure of the present disclosure may be synthesized, using any suitable method.

In some embodiments, effector proteins described herein can be isolated and purified for use in compositions, systems, and/or methods described herein. Methods described here can include the step of isolating the effector protein described herein. Compositions and/or systems described herein can further comprise a purification tag that can be attached to an effector protein, or a nucleic acid encoding for a purification tag that can be attached to a nucleic acid encoding for an effector protein as described herein. A purification tag, as used herein, can be an amino acid sequence which can attach or bind with high affinity to a separation substrate and assist in isolating the protein of interest from its environment, which can be its biological source, such as a cell lysate. Attachment of the purification tag can be at the N or C terminus of the effector protein. Furthermore, an amino acid sequence recognized by a protease or a nucleic acid encoding for an amino acid sequence recognized by a protease, such as TEV protease or the HRV3C protease can be inserted between the purification tag and the effector protein, such that biochemical cleavage of the sequence with the protease after initial purification liberates the purification tag. Purification and/or isolation can be through high performance liquid chromatography (HPLC), exclusion chromatography, gel electrophoresis, affinity chromatography, or other purification technique. In some embodiments, purification tags can be a fluorescent protein, e.g., green fluorescent protein (GFP), yellow fluorescent protein (YFP), red fluorescent protein (RFP), cyan fluorescent protein (CFP), mCherry, tdTomato, and the like; a histidine tag, e.g., a 6×His tag; a hemagglutinin (HA) tag; a FLAG tag; a Myc tag; and the like.

For example, in some embodiments, effector proteins described herein are isolated from cell lysate. In some embodiments, the compositions described herein can comprise 20% or more by weight, 75% or more by weight, 95% or more by weight, or 99.5% or more by weight of an effector protein, related to the method of preparation of compositions described herein and its purification thereof, wherein percentages can be upon total protein content in relation to contaminants. Thus, in some cases, an effector protein described herein is at least 80% pure, at least 85% pure, at least 90% pure, at least 95% pure, at least 98% pure, or at least 99% pure (e.g., free of contaminants, non-engineered polypeptide proteins or other macromolecules, etc.).

PAM sequences

In some embodiments, effector proteins cleave or nick a target nucleic acid within or near a protospacer adjacent motif (PAM) sequence of the target nucleic acid. In some embodiments, the target nucleic acid is a double stranded nucleic acid comprising a target strand and a non-target strand. In some embodiments, cleavage occurs within 1, 2, 3, 4, 5, 6, 7, 8, 9 or 10 nucleosides of a 5′ or 3′ terminus of a PAM sequence. In some embodiments, effector proteins described herein recognize a PAM sequence. In some embodiments, recognizing a PAM sequence comprises interacting with a sequence adjacent to the PAM. A target nucleic acid may comprise a PAM sequence adjacent to a sequence that is complementary to a guide nucleic acid spacer sequence. In some embodiments, effector proteins do not require a PAM sequence to cleave or a nick a target nucleic acid.

In some embodiments, a target nucleic acid is a single stranded target nucleic acid comprising a target sequence. Accordingly, in some embodiments, the single stranded target nucleic acid comprises a PAM sequence described herein that is adjacent (e.g., within 1, 2, 3, 4, 5, 6, 7, 8, 9, 10, 15, 20, 25, 30, 35, 40, 45, or 50 nucleotides) or directly adjacent to the target sequence. In some embodiments, an RNP cleaves the single stranded target nucleic acid.

In some embodiments, a target nucleic acid is a double stranded nucleic acid comprising a target strand and a non-target strand, wherein the target strand comprises a target sequence. In some embodiments, the PAM sequence is located on the target strand. In some embodiments, the PAM sequence is located on the non-target strand. In some embodiments, the PAM sequence described herein is adjacent (e.g., within 1, 2, 3, 4, 5, 6, 7, 8, 9, 10, 15, 20, 25, 30, 35, 40, 45, or 50 nucleotides) to the target sequence on the target strand or the non-target strand. In some embodiments, such a PAM described herein is directly adjacent to the target sequence on the target strand or the non-target strand. In some embodiments, an RNP cleaves the target strand or the non-target strand. In some embodiments, the RNP cleaves both, the target strand and the non-target strand. In some embodiments, an RNP recognizes the PAM sequence, and hybridizes to a target sequence of the target nucleic acid. In some embodiments, the RNP cleaves the target nucleic acid, wherein the RNP has recognized the PAM sequence and is hybridized to the target sequence.

In some embodiments, an effector protein described herein, or a multimeric complex thereof, recognizes a PAM on a target nucleic acid. In some embodiments, multiple effector proteins of the multimeric complex recognize a PAM on a target nucleic acid. In some embodiments, at least two of the multiple effector proteins recognize the same PAM sequence. In some embodiments, at least two of the multiple effector proteins recognize different PAM sequences. In some embodiments, only one effector protein of the multimeric complex recognizes a PAM on a target nucleic acid.

An effector protein of the present disclosure, or a multimeric complex thereof, may cleave or nick a target nucleic acid within or near a protospacer adjacent motif (PAM) sequence of the target nucleic acid. In some embodiments, cleavage occurs within 1, 2, 3, 4, 5, 6, 7, 8, 9 or 10 nucleotides of a 5′ or 3′ terminus of a PAM sequence

In some embodiments, the PAM sequence comprises a nucleotide sequence in TABLE 2. In some embodiments, the PAM sequence comprises a nucleotide sequence that is at least 80%, at least 85%, at least 90%, at least 95%, at least 98% or 100% identical to a nucleotide sequence in TABLE 2. In some embodiments, the nucleotide sequence of the PAM sequence is at least 80%, at least 85%, at least 90%, at least 95%, at least 98% or 100% identical to a nucleotide sequence in TABLE 2. In some embodiments, the PAM sequence comprises at least 3, at least 4, at least 5, at least 6, or at least 7 contiguous nucleotides of a nucleotide sequence in TABLE 2.

Consensus PAM sequences as listed in TABLE 2 comprise, in some instances, an ambiguity code, wherein one or more nucleotides can appear in the stated sequence location. Ambiguity codes are listed in TABLE 3.

### II. Guide Nucleic Acids

The compositions, systems, and methods of the present disclosure may comprise a guide nucleic acid or a use thereof. A guide nucleic acid may comprise a sequence that is bound by an effector protein. Guide nucleic acids may comprise DNA, RNA, or a combination thereof (e.g., RNA with a thymine base). Guide nucleic acids may be referred to herein as a guide RNA (gRNA). However, a guide RNA is not limited to ribonucleotides, but may comprise deoxyribonucleotides and other chemically modified nucleotides. Accordingly, a guide nucleic acid, as well as any components thereof (e.g., spacer sequence, repeat sequence, linker nucleotide sequence, etc.) may comprise one or more deoxyribonucleotides, ribonucleotides, biochemically or chemically modified nucleotides (e.g., one or more engineered modifications as described herein), or any combinations thereof. Guide nucleic acids may include a chemically modified nucleobase or phosphate backbone.

A guide nucleic acid may comprise a naturally occurring guide nucleic acid. A guide nucleic acid may comprise a non-naturally occurring guide nucleic acid, including a guide nucleic acid that is designed to contain a chemical or biochemical modification. The sequence of a guide nucleic acid may comprise two or more heterologous sequences. Guide RNAs may be chemically synthesized or recombinantly produced.

Guide nucleic acids, when complexed with an effector protein, may bring the effector protein into proximity of a target nucleic acid. Sufficient conditions for hybridization of a guide nucleic acid to a target nucleic acid and/or for binding of a guide nucleic acid to an effector protein include in vivo physiological conditions of a desired cell type or in vitro conditions sufficient for assaying catalytic activity of a protein, polypeptide or peptide described herein, such as the nuclease activity of an effector protein.

In some embodiments, fusion effector proteins are targeted by a guide nucleic acid (e.g., a guide RNA) to a specific location in the target nucleic acid where they exert locus-specific regulation. Non-limiting examples of locus-specific regulation include blocking RNA polymerase binding to a promoter (which selectively inhibits transcription activator function), and/or modifying local chromatin (e.g., when a fusion sequence is used that modifies the target nucleic acid or modifies a protein associated with the target nucleic acid). The guide nucleic acid may hybridize to a target nucleic acid (e.g., a single strand of a target nucleic acid) or a portion thereof, an amplicon thereof, or a portion thereof. The target nucleic acid, in some embodiments, comprises a mutation. In some embodiments, the mutation is located in a non-coding region of a gene. By way of non-limiting example, a guide nucleic acid may hybridize to a target nucleic acid, such as DNA or RNA, from a cancer gene or gene associated with a genetic disorder, or an amplicon thereof, as described herein.

In some cases, an effector protein cleaves a precursor RNA (“pre-crRNA”) to produce a guide RNA, also referred to as a “mature guide RNA.” An effector protein that cleaves pre-crRNA to produce a mature guide RNA is said to have pre-crRNA processing activity. In some cases, a repeat sequence of a guide RNA comprises mutations or truncations relative to respective regions in a corresponding pre-crRNA.

The guide nucleic acid may comprise a first region complementary to a target nucleic acid (FR1) and a second region that is not complementary to the target nucleic acid (FR2). In some cases, FR1 is located 5′ to FR2 (FR1-FR2). In some cases, FR2 is located 5′ to FR1 (FR2-FR1).

In some cases, the guide nucleic acid comprises 10, 11, 12, 13, 14, 15, 16, 17, 18, 19, 20, 21, 22, 23, 24, 25, 26, 27, 28, 29, or 30 linked nucleosides. In general, a guide nucleic acid comprises at least linked nucleosides. In some embodiments, a guide nucleic acid comprises at least 25 linked nucleosides. A guide nucleic acid may comprise 10 to 50 linked nucleosides. In some cases, the guide nucleic acid comprises or consists essentially of about 12 to about 80 linked nucleosides, about 12 to about 50, about 12 to about 45, about 12 to about 40, about 12 to about 35, about 12 to about 30, about 12 to about 25, from about 12 to about 20, about 12 to about 19, about 19 to about 20, about 19 to about 25, about 19 to about 30, about 19 to about 35, about 19 to about 40, about 19 to about 45, about 19 to about 50, about 19 to about 60, about 20 to about 25, about 20 to about 30, about 20 to about 35, about 20 to about 40, about 20 to about 45, about 20 to about 50, or about 20 to about 60 linked nucleosides. In some cases, the guide nucleic acid has about 10 to about 60, about 20 to about 50, or about 30 to about 40 linked nucleosides.

A guide nucleic acid may comprise a CRISPR RNA (crRNA), a short-complementarity untranslated RNA (scoutRNA), an associated trans-activating RNA (tracrRNA) sequence or a combination thereof. The combination of a crRNA with a tracrRNA sequence may be referred to herein as a single guide RNA (sgRNA), wherein the crRNA and the tracrRNA sequence are covalently linked. In some embodiments, the crRNA and tracrRNA sequence are linked by a phosphodiester bond. In some instances, the crRNA and tracrRNA sequence are linked by one or more linked nucleotides.

### Spacer Sequence

Guide nucleic acids described herein may comprise one or more spacer sequences. In some embodiments, a spacer sequence is capable of hybridizing to a target sequence of a target nucleic acid. Exemplary hybridization conditions are described herein. In some embodiments, the spacer sequence may function to direct an RNP complex comprising the guide nucleic acid to the target nucleic acid for detection and/or modification. The spacer sequence may function to direct a RNP to the target nucleic acid for detection and/or modification. A spacer sequence may be complementary to a target sequence that is adjacent to a PAM that is recognizable by an effector protein described herein.

The spacer sequence may comprise complementarity with (e.g., hybridize to) a target sequence of a target nucleic acid. In some embodiments, a spacer sequence comprises at least 5 to about 50, at least 5 to about 25, at least about 10 to at least about 25, or at least about 15 to about 25 linked nucleotides. In some cases, the spacer sequence is 15-28 linked nucleosides in length. In some cases, the spacer sequence is 15-26, 15-24, 15-22, 15-20, 15-18, 16-28, 16-26, 16-24, 16-22, 16-20, 16-18, 17-26, 17-24, 17-22, 17-20, 17-18, 18-26, 18-24, or 18-22 linked nucleosides in length. In some cases, the spacer sequence is 18-24 linked nucleosides in length. In some cases, the spacer sequence is at least 15 linked nucleosides in length. In some cases, the spacer sequence is at least 16, 18, 20, or 22 linked nucleosides in length. In some cases, the spacer sequence comprises at least 10, 11, 12, 13, 14, 15, 16, 17, 18, 19, 20, 21, 22, 23, 24, 25, 26, 27, 28, 29, or 30 nucleotides. In some cases, the spacer sequence is at least 17 linked nucleosides in length. In some cases, the spacer sequence is at least 18 linked nucleosides in length. In some cases, the spacer sequence is at least 20 linked nucleosides in length. In some embodiments, a spacer sequence comprises at least 5 to about 50 contiguous nucleotides that are complementary to a target sequence in a target nucleic acid. In some cases, the spacer sequence comprises at least 15 contiguous nucleobases that are complementary to the target nucleic acid.

Typically, the repeat sequence is adjacent to the spacer sequence. For example, a guide RNA that interacts with an effector protein comprises a repeat sequence that is 3′ of the spacer sequence; or a guide RNA that interacts with an effector protein comprises a repeat sequence that is 5′ of the spacer sequence. In some embodiments, a spacer sequence precedes a repeat sequence in a 5′ to 3′ direction. Generally, a guide RNA that interacts with an effector protein comprises a spacer sequence that is 5′ to a repeat sequence. In some embodiments, the spacer sequence(s) and the repeat sequence(s) of the guide nucleic acid are present within the same molecule. In some embodiments, the spacer(s) and repeat sequence(s) are linked directly to one another. In some embodiments, a linker is present between the spacer(s) and repeat sequences. Linkers may be any suitable linker. In some embodiments, the spacer sequence(s) and the repeat sequence(s) of the guide nucleic acid are present in separate molecules, which are joined to one another by base pairing interactions.

In some embodiments, a spacer sequence comprises a nucleotide sequence that is at least 65%, at least 70%, at least 75%, at least 80%, at least 85%, at least 90%, at least 95% or 100% complementary to a target sequence of a target nucleic acid. A spacer sequence is capable of hybridizing to an equal length portion of a target nucleic acid (e.g., a target sequence). In some embodiments, a target nucleic acid is a nucleic acid associated with a disease or syndrome described herein. In some embodiments, a target nucleic acid, such as DNA or RNA, may be a cancer gene or gene associated with a genetic disorder, or an amplicon thereof, as described herein. In some embodiments, a target nucleic acid is a gene selected from TABLE 8. In some embodiments, a spacer sequence comprises a sequence that is at least 65%, at least 70%, at least 75%, at least 80%, at least 85%, at least 90%, at least 95% or 100% complementary to a target sequence of a target nucleic acid selected from TABLE 8. In some embodiments, a target nucleic acid is a nucleic acid associated with a disease or syndrome set forth in TABLE 7. In some embodiments, a spacer sequence comprises a sequence that is at least 65%, at least 70%, at least 75%, at least 80%, at least 85%, at least 90%, at least 95% or 100% complementary to a target sequence of a target nucleic acid associated with a disease or syndrome set forth in TABLE 7. In some embodiments, the spacer sequence comprises at least 10, 11, 12, 13, 14, 15, 16, 17, 18, 19, 20, 21, 22, 23, 24, 25, 26, 27, 28, 29, or 30 contiguous nucleotides that are capable of hybridizing to the target sequence. In some embodiments, the spacer sequence comprises at least 10, 11, 12, 13, 14, 15, 16, 17, 18, 19, 20, 21, 22, 23, 24, 25, 26, 27, 28, 29, or 30 contiguous nucleotides that are complementary to the target sequence.

It is understood that the sequence of a spacer sequence need not be 100% complementary to that of a target sequence of a target nucleic acid to hybridize or hybridize specifically to the target sequence. Accordingly, in some embodiments, a spacer sequence may comprise a nucleotide sequence that may have partial complementarity with (e.g., hybridize to) an equal length of a target sequence of a target nucleic acid. The guide nucleic acid may comprise at least one uracil between nucleic acid residues 5 to 20 of the spacer sequence that is not complementary to the corresponding nucleoside of the target sequence. The guide nucleic acid may comprise at least one uracil between nucleic acid residues 5 to 9, 10 to 14, or 15 to 20 of the spacer sequence that is not complementary to the corresponding nucleoside of the target sequence. In some cases, the region of the target nucleic acid that is complementary to the spacer sequence comprises an epigenetic modification or a post-transcriptional modification. In some cases, the epigenetic modification comprises acetylation, methylation, or thiol modification.

### Repeat Sequence

Guide nucleic acids described herein may comprise one or more repeat sequences. In some embodiments, a repeat sequence comprises a nucleotide sequence that is not complementary to a target sequence of a target nucleic acid. In some embodiments, a repeat sequence comprises a nucleotide sequence that may interact with an effector protein. In some embodiments, a repeat sequence is connected to another sequence of a guide nucleic acid that is capable of non-covalently interacting with an effector protein. In some embodiments, a repeat sequence includes a nucleotide sequence that is capable of forming a guide nucleic acid-effector protein complex (e.g., a RNP complex).

In some embodiments, the repeat sequence is between 10 and 50, 12 and 48, 14 and 46, 16 and 44, and 18 and 42 nucleotides in length.

In some embodiments, the repeat sequence comprises two sequences that are complementary to each other and hybridize to form a double stranded RNA duplex (dsRNA duplex). In some embodiments, the two sequences are not directly linked and hybridize to form a stem loop structure. In some embodiments, the dsRNA duplex comprises 5, 10, 15, 20 or 25 base pairs (bp). In some embodiments, not all nucleotides of the dsRNA duplex are paired, and therefore the duplex forming sequence may include a bulge. In some embodiments, the repeat sequence comprises a hairpin or stem-loop structure, optionally at the 5′ portion of the repeat sequence. In some embodiments, a strand of the stem portion comprises a sequence and the other strand of the stem portion comprises a sequence that is, at least partially, complementary. In some embodiments, such sequences may have 65% to 100% complementarity (e.g., 65%, 70%, 75%, 80%, 85%, 90%, 95%, or 100% complementarity). In some embodiments, a guide nucleic acid comprises nucleotide sequence that when involved in hybridization events may hybridize over one or more segments such that intervening or adjacent segments are not involved in the hybridization event (e.g., a bulge, a loop structure or hairpin structure, etc.).

TABLE 4 provides exemplary guide nucleic acids having repeat sequences, as well as compositions comprising an effector protein and a guide nucleic acid having a repeat sequence. For example, each row in TABLE 4 represents an exemplary composition. In some embodiments, the repeat sequence comprises a nucleotide sequence that interacts with the effector protein. In some embodiments, the repeat sequence comprises a nucleotide sequence as described herein (e.g., TABLE 4). In some embodiments, the guide nucleic acid of the composition comprises a repeat sequence of any one of SEQ ID NOS: 123-165 as shown in TABLE 4. In some embodiments, the repeat sequence of the guide nucleic acid is at least 65%, at least 70%, at least 75%, at least 80%, at least 85%, at least 90%, at least 95%, or 100% identical to any one of SEQ ID NOS: 123-165 as shown in TABLE 4.

In some embodiments, a repeat sequence comprises a nucleotide sequence that is at least 65% identical to any one of the SEQ ID NO: 123-165. In some embodiments, a repeat sequence comprises a nucleotide sequence that is at least 70% identical to any one of the SEQ ID NO: 123-165. In some embodiments, a repeat sequence comprises a nucleotide sequence that is at least 75% identical to any one of the SEQ ID NO: 123-165. In some embodiments, a repeat sequence comprises a nucleotide sequence that is at least 80% identical to any one of the SEQ ID NO: 123-165. In some embodiments, a repeat sequence comprises a nucleotide sequence that is at least 85% identical to any one of the SEQ ID NO: 123-165. In some embodiments, a repeat sequence comprises a nucleotide sequence that is at least 90% identical to any one of the SEQ ID NO: 123-165. In some embodiments, a repeat sequence comprises a nucleotide sequence that is at least 95% identical to any one of the SEQ ID NO: 123-165. In some embodiments, a repeat sequence comprises a nucleotide sequence that is at least 97% identical to any one of the SEQ ID NO: 123-165. In some embodiments, a repeat sequence comprises a nucleotide sequence that is at least 99% identical to any one of the SEQ ID NO: 123-165. In some embodiments, a repeat sequence comprises a nucleotide sequence that is identical to any one of the SEQ ID NO: 123-165.

In some embodiments, the guide nucleic acid comprises a nucleotide sequence in TABLE 4. In some embodiments, the guide nucleic acid comprises a nucleotide sequence that is at least 80%, at least 85%, at least 90%, at least 95%, at least 98% or 100% identical to a nucleotide sequence in TABLE 4. In some embodiments, guide nucleic acid comprises at least 8, at least 9, at least 10, at least 11, at least 12, at least 13, at least 14, at least 15, at least 16, or at least 17 contiguous nucleotides of a nucleotide sequence in TABLE 4.

### Linker for Nucleic Acids

In some embodiments, a guide nucleic acid for use with compositions, systems, and methods described herein comprises one or more linkers, or a nucleic acid encoding one or more linkers. In some embodiments, the guide nucleic acid comprises at least one, at least two, at least three, at least four, at least five, at least six, at least seven, at least eight, at least nine, or at least ten linkers. In some embodiments, the guide nucleic acid comprises one, two, three, four, five, six, seven, eight, nine, or ten linkers. In some embodiments, the guide nucleic acid comprises more than one linker. In some embodiments, at least two of the more than one linker are the same. In some embodiments, at least two of the more than one linker are not same.

In some embodiments, a linker comprises one to ten, one to seven, one to five, one to three, two to ten, two to eight, two to six, two to four, three to ten, three to seven, three to five, four to ten, four to eight, four to six, five to ten, five to seven, six to ten, six to eight, seven to ten, or eight to ten linked nucleotides. In some embodiments, the linker comprises one, two, three, four, five, six, seven, eight, nine, or ten linked nucleotides. In some embodiments, a linker comprises a nucleotide sequence of 5′-GAAA-3′.

In some embodiments, the guide nucleic acid comprises one or more linkers connecting one or more repeat sequences and one or more spacer sequences.

### III. A Single Nucleic Acid System

In some embodiments, compositions, systems and methods described herein comprise a single nucleic acid system comprising a guide nucleic acid or a nucleotide sequence encoding the guide nucleic acid, and one or more effector proteins or a nucleotide sequence encoding the one or more effector proteins. In some embodiments, a second region (FR2) of the guide nucleic acid non-covalently interacts with the one or more polypeptides described herein. In some embodiments, a first region (FR1) of the guide nucleic acid hybridizes with a target sequence of the target nucleic acid. In the single nucleic acid system having a complex of the guide nucleic acid and the effector protein, the effector protein is not transactivated by the guide nucleic acid. In other words, activity of effector protein does not require binding to a second non-target nucleic acid molecule. An exemplary guide nucleic acid for a single nucleic acid system is a crRNA or a sgRNA. In general, the FR1 is located 5′ of the FR2.

crRNA

In general, the guide nucleic acid comprises a CRISPR RNA (crRNA), at least a portion of which is complementary to a target sequence of a target nucleic acid. In some embodiments, CRISPR RNA or crRNA is a type of guide nucleic acid, wherein the nucleic acid is RNA comprising a first sequence, often referred to herein as a spacer sequence, that hybridizes to a target sequence of a target nucleic acid, and a second sequence that is capable of being connected to an effector protein by either a) hybridization to a portion of a tracrRNA or b) being non-covalently bound by an effector protein.

In some embodiments, the crRNA comprises a sequence that is bound by an effector protein. In some embodiments, the crRNA of the guide nucleic acid comprises a repeat sequence and a spacer sequence, wherein the repeat sequence binds to the effector protein and the spacer sequence hybridizes to a target sequence of the target nucleic acid. The repeat sequence of the crRNA may interact with an effector protein, allowing for the guide nucleic acid and the effector protein to form a complex. In some embodiments, the repeat sequence and the spacer sequences are directly connected to each other (e.g., covalent bond (phosphodiester bond)). In some embodiments, the repeat sequence and the spacer sequence are connected by a linker. In general, a crRNA comprises a spacer sequence that hybridizes to a target sequence of a target nucleic acid, and a repeat sequence that interacts with a tracrRNA or an effector protein. In some embodiments, the composition does not comprise a tracrRNA.

Guide nucleic acids and portions thereof may be found in or identified from a CRISPR array present in the genome of a host organism. A crRNA may be the product of processing of a longer precursor CRISPR RNA (pre-crRNA) transcribed from the CRISPR array by cleavage of the pre-crRNA within each direct repeat sequence to afford shorter, mature crRNAs. A crRNA may be generated by a variety of mechanisms, including the use of dedicated endonucleases (e.g., Cas6 or Cas5d in Type I and III systems), coupling of a host endonuclease (e.g., RNase III) with tracrRNA (Type II systems), or a ribonuclease activity endogenous to the effector protein itself (e.g., Cpfl, from Type V systems). A crRNA may also be specifically generated outside of processing of a pre-crRNA and individually contacted to an effector protein in vivo or in vitro.

In some embodiments, a crRNA is useful as a single nucleic acid system for compositions, methods, and systems described herein or as part of a single nucleic acid system for compositions, methods, and systems described herein. In some embodiments, a crRNA is useful as part of a single nucleic acid system for compositions, methods, and systems described herein. In some embodiments, a single nucleic acid system comprises a guide nucleic acid comprising a crRNA linked to another nucleotide sequence that is capable of being non-covalently bond by an effector protein. In such embodiments, a repeat sequence of a crRNA can be linked to an additional nucleotide sequence. In some embodiments, a single nucleic acid system comprises a guide nucleic acid comprising a crRNA and an additional nucleotide sequence.

A crRNA may include deoxyribonucleosides, ribonucleosides, chemically modified nucleosides, or any combination thereof. In some embodiments, a crRNA comprises about: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 linked nucleotides. In some embodiments, a crRNA comprises at least: 10, 15, 20, 25, 30, 35, 40, 45, 50, 55, 60 linked nucleotides. In some embodiments, the length of the crRNA is about 20 to about 120 linked nucleotides. In some embodiments, the length of a crRNA is about 20 to about 100, about 30 to about 100, about 40 to about 100, about 40 to about 90, about 40 to about 80, about 40 to about 70, about 40 to about 60, about 40 to about 50, about 50 to about 90, about 50 to about 80, about 50 to about 70, or about 50 to about 60 linked nucleotides. In some embodiments, the length of a crRNA is about 20, about 25, about 30, about 35, about 40, about 45, about 50, about 55, about 60, about 65, about 70 or about 75 linked nucleotides.

sgRNA

In some embodiments, a guide nucleic acid comprises a sgRNA. In some embodiments, a guide nucleic acid is a sgRNA. In some embodiments, a sgRNA comprises a first sequence (FR1) and a second sequence (FR2). In some embodiments, the first sequence (FR1) and the second sequence (FR2) are directly connected to each other (e.g., covalent bond (phosphodiester bond)). In some embodiments, the first sequence (FR1) and the second sequence (FR2) are connected by a linker. In general, the first sequence (FR1) is 5′ of the second sequence (FR2).

In some embodiments, the crRNA covalently linked to another nucleic acid sequence, also referred to herein as a single guide RNA (sgRNA). In some embodiments, the crRNA and an additional nucleic acid sequence are linked by a phosphodiester bond. In some embodiments, the additional nucleic acid sequence may be attached by a linker to the crRNA. In some embodiments, the additional nucleic acid sequence is not transactivated or transactivating in systems, methods, and compositions described herein.

In some embodiments, the crRNA and the tracrRNA sequence are covalently linked, also referred to herein as a single guide RNA (sgRNA). Accordingly, in some embodiments, the guide RNA comprises a tracrRNA sequence. The tracrRNA sequence may be linked to a crRNA to form a composite gRNA. In some cases, the crRNA and the tracrRNA sequence are provided as a single nucleic acid (e.g., covalently linked). In some embodiments, the crRNA and tracrRNA sequence are linked by a phosphodiester bond. The tracrRNA sequence may be attached by an artificial linker to a guide nucleic acid. In some embodiments, the crRNA and tracrRNA sequence are linked by one or more linked nucleotides.

In some embodiments, a second sequence (FR2) of the sgRNA is bound by the effector protein. In some embodiments, the guide nucleic acid comprises a trans-activating CRISPR RNA (tracrRNA) sequence that interacts with the effector protein. In some embodiments, the crRNA is covalently linked to an additional nucleic acid (e.g., a tracrRNA) sequence that is bound by the effector protein.

In some embodiments, the guide nucleic acid is an sgRNA comprising a nucleotide sequence in TABLE 5, TABLE 6, and TABLE 11. In some embodiments, the guide nucleic acid is an sgRNA comprising a nucleotide sequence that is at least 80%, at least 85%, at least 90%, at least 95%, at least 98% or 100% identical to a nucleotide sequence in TABLE 5, TABLE 6, and TABLE 11. In some embodiments, the nucleotide sequence of the guide nucleic acid is at least 80%, at least 85%, at least 90%, at least 95%, at least 98% or 100% identical to a nucleotide sequence in TABLE 5, TABLE 6, and TABLE 11. In some embodiments, guide nucleic acid comprises at least 8, at least 9, at least 10, at least 11, at least 12, at least 13, at least 14, at least 15, at least 16, at least 17, at least 18, at least 19, or at least 20 contiguous nucleotides of a nucleotide sequence in TABLE 5, TABLE 6, and TABLE 11. In some embodiments, guide nucleic acid comprises at least 20, at least 30, at least 40, at least 50, at least 60, at least 70, at least 80, at least 90, at least 100, or at least 110 contiguous nucleotides of a nucleotide sequence in TABLE 5, TABLE 6, and TABLE 11.

TABLE 5, TABLE 6, and TABLE 11 provide exemplary guide nucleic acids (sgRNA), as well as compositions comprising an effector protein and an sgRNA. For example, each row in TABLE 5, TABLE 6, and TABLE 11 represents an exemplary composition. In some embodiments, the sgRNA comprises a nucleotide sequence that interacts with the effector protein. In some embodiments, the sgRNA comprises a nucleotide sequence as described herein (e.g., TABLE 5, TABLE 6, and TABLE 11). In some embodiments, the sgRNA of the composition comprises a nucleotide sequence of any one of SEQ ID NOs: 166-227, 327-425 and 436-498 as shown in TABLE 5, TABLE 6, and TABLE 11. In some embodiments, the nucleotide sequence of the sgRNA is at least 65%, at least 70%, at least 75%, at least 80%, at least 85%, at least 90%, at least 95%, or 100% identical to any one of SEQ ID NOs: 166-227, 327-425 and 436-498 as shown in TABLE 5, TABLE 6, and TABLE 11. In some embodiments, a guide nucleic acid comprises a nucleotide sequence that is at least 65% identical to any one of SEQ ID NOs: 166-227, 327-425 and 436-498. In some embodiments, a guide nucleic acid comprises a nucleotide sequence that is at least 70% identical to any one of SEQ ID NOs: 166-227, 327-425 and 436-498. In some embodiments, a guide nucleic acid comprises a nucleotide sequence that is at least 75% identical to any one of SEQ ID NOs: 166-227, 327-425 and 436-498. In some embodiments, a guide nucleic acid comprises a nucleotide sequence that is at least 80% identical to any one of SEQ ID NOs: 166-227, 327-425 and 436-498. In some embodiments, a guide nucleic acid comprises a nucleotide sequence that is at least 85% identical to any one of SEQ ID NOs: 166-227, 327-425 and 436-498. In some embodiments, a guide nucleic acid comprises a nucleotide sequence that is at least 90% identical to any one of SEQ ID NOs: 166-227, 327-425 and 436-498. In some embodiments, a guide nucleic acid comprises a nucleotide sequence that is at least 95% identical to any one of SEQ ID NOs: 166-227, 327-425 and 436-498. In some embodiments, a guide nucleic acid comprises a nucleotide sequence that is at least 97% identical to any one of SEQ ID NOs: 166-227, 327-425 and 436-498. In some embodiments, a guide nucleic acid comprises a nucleotide sequence that is at least 99% identical to any one of SEQ ID NOs: 166-227, 327-425 and 436-498. In some embodiments, a guide nucleic acid comprises a nucleotide sequence that is identical to any one of SEQ ID NOs: 166-227, 327-425 and 436-498.

### IV. A Dual Nucleic Acid System

In some embodiments, the crRNA and a tracrRNA are in a dual nucleic acid system and are not linked by a covalent bond. In such a dual nucleic acid system, the crRNA can be connected to the effector protein by hybridization to a portion of the tracrRNA, and the tracrRNA includes a separate portion that is bound by the effector protein. In some embodiments, the tracrRNA comprises a repeat hybridization sequence that is capable of hybridizing with an equal length portion of a crRNA to form a tracrRNA-crRNA duplex, wherein the equal length portion of the crRNA does not include a spacer sequence of the crRNA, and wherein the spacer sequence is capable of hybridizing to a target sequence of the target nucleic acid.

In the dual nucleic acid system having a complex of the guide nucleic acid, tracrRNA, and the effector protein, the effector protein is transactivated by the tracrRNA. In other words, activity of effector protein requires binding to a tracrRNA molecule. In some embodiments, a tracrRNA and/or tracrRNA-crRNA duplex may form a secondary structure that facilitates the binding of an effector protein to a tracrRNA or a tracrRNA-crRNA. In some embodiments, a transactivated tracrRNA is enabled to have a binding and/or nuclease activity on a target nucleic acid, by a tracrRNA or a tracrRNA-crRNA duplex by any of the effector proteins described herein. In some embodiments, the tracrRNA or a tracrRNA-crRNA duplex transactivates effector proteins described herein (e.g., enabled to have a binding and/or nuclease activity on a target nucleic acid).

tracrRNA

In some embodiments, a crRNA and tracrRNA function as two separate, unlinked molecules. In some embodiments, compositions comprise a tracrRNA that is separate from, but forms a complex with a crRNA to form a gRNA system.

The tracrRNA may include deoxyribonucleosides, ribonucleosides, chemically modified nucleosides, or any combination thereof. The tracrRNA may be separate from, but form a complex with, a guide nucleic acid and an effector protein. In some embodiments, the crRNA and the tracrRNA are separate polynucleotides. A tracrRNA may include a nucleotide sequence that hybridizes with a portion of a guide nucleic acid. In some embodiments, a tracrRNA comprises a repeat hybridization sequence that hybridizes with a portion of a guide nucleic acid. In some embodiments, a repeat hybridization sequence is at the 3′ end of a tracrRNA. In some embodiments, a repeat hybridization sequence may have a length of about 1, about 2, about 3, about 4, about 5, about 6, about 7, about 8, about 9, about 10, about 12, about 14, about 16, about 18, or about 20 linked nucleotides. In some embodiments, the length of the repeat hybridization sequence is 1 to 20 linked nucleotides.

In general, a tracrRNA comprises a nucleotide sequence that is bound by an effector protein. A tracrRNA may comprise at least one secondary structure (e.g., hairpin loop) that facilitates the binding of an effector protein to a guide nucleic acid and/or modification activity of an effector protein on a target nucleic acid. A tracrRNA may include a repeat sequence and a hairpin region. The repeat sequence may hybridize to all or part of the repeat sequence of a crRNA. The repeat sequence may be positioned 3′ of the hairpin region. The repeat sequence may be positioned 5′ of the hairpin region. The hairpin region may include a first sequence, a second sequence that is reverse complementary to the first sequence, and a stem-loop linking the first sequence and the second sequence,

In some embodiments, the length of a tracrRNA is not greater than 50, 56, 68, 71, 73, 95, or 105 linked nucleosides. In some embodiments, the length of a tracrRNA is about 30 to about 120 linked nucleosides. In some embodiments, the length of a tracrRNA is about 50 to about 105, about 50 to about 95, about 50 to about 73, about 50 to about 71, about 50 to about 68, or about 50 to about 56 linked nucleosides. In some embodiments, the length of a tracrRNA is 56 to 105 linked nucleosides, from 56 to 105 linked nucleosides, 68 to 105 linked nucleosides, 71 to 105 linked nucleosides, 73 to 105 linked nucleosides, or 95 to 105 linked nucleosides. In some embodiments, the length of a tracrRNA is 40 to 60 nucleotides. In some embodiments, the length of a tracrRNA is 50, 56, 68, 71, 73, 95, or 105 linked nucleosides. In some embodiments, the length of a tracrRNA is 50 nucleotides.

In some embodiments, compositions do not comprise a tracrRNA. In some embodiments, methods do not comprise the use of a tracrRNA. In some embodiments, the guide RNA does not comprise a tracrRNA. In some cases, an effector protein does not require a tracrRNA to locate and/or cleave a target nucleic acid.

### Exemplary Guide Nucleic Acids and Compositions

In some embodiments, the guide nucleic acid comprises a nucleotide sequence as described herein (e.g., TABLES 4-6 and 11). Such nucleotide sequences described herein (e.g., TABLES 4-6 and 11) may be described as a nucleotide sequence of either DNA or RNA, however, no matter the form the sequence is described, it is readily understood that such nucleotide sequences can be revised to be RNA or DNA, as needed, for describing a sequence within a guide nucleic acid itself or the sequence that encodes a guide nucleic acid, such as a nucleotide sequence described herein for a viral vector. Similarly, disclosure of the nucleotide sequences described herein (e.g., TABLES 4-6 and 11) also discloses the complementary nucleotide sequence, the reverse nucleotide sequence, and the reverse complement nucleotide sequence, any one of which can be a nucleotide sequence for use in a guide nucleic acid as described herein. In some embodiments, a guide nucleic acid sequence(s) comprises one or more nucleotide alterations at one or more positions in any one of the sequences described herein (e.g., TABLES 4-6 and 11). Alternative nucleotides can be any one or more of A, C, G, T or U, or a deletion, or an insertion.

In some embodiments, the guide nucleic acid comprises a nucleotide sequence in TABLE 5, TABLE 6 and TABLE 11. In some embodiments, the guide nucleic acid comprises a nucleotide sequence that is at least 80%, at least 85%, at least 90%, at least 95%, at least 98% or 100% identical to a nucleotide sequence in TABLE 5, TABLE 6 and TABLE 11. In some embodiments, the nucleotide sequence of the guide nucleic acid is at least 80%, at least 85%, at least 90%, at least 95%, at least 98% or 100% identical to a nucleotide sequence in TABLE 5, TABLE 6 and TABLE 11. In some embodiments, guide nucleic acids comprise at least 8, at least 9, at least 10, at least 11, at least 12, at least 13, at least 14, at least 15, at least 16, at least 17, at least 18, at least 19, or at least 20 contiguous nucleotides of a nucleotide sequence in TABLE 5, TABLE 6 and TABLE 11. In some embodiments, guide nucleic acids comprise at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, or at least 220 contiguous nucleotides of a nucleotide sequence in TABLE 5, TABLE 6 and TABLE 11. In some embodiments, the guide nucleic acid comprises a nucleotide sequence in TABLE 5, TABLE 6 and TABLE 11, minus the 5′ most 20, 21, 22, 23, 24 or 25 nucleotides.

TABLE 6 provides exemplary guide nucleic acids (represented by DNA or RNA sequences), as well as compositions comprising an effector protein and a guide nucleic acid. For example, each row in TABLE 6 represents an exemplary composition. Similarly, TABLE 5 and TABLE 11 provide exemplary guide nucleic acids (represented by RNA sequences), as well as compositions comprising an effector protein and a guide nucleic acid. For example, each row in TABLE 5 and TABLE 11 represents an exemplary composition. In some embodiments, the guide nucleic acid comprises a nucleotide sequence that interacts with the effector protein. In some embodiments, the guide nucleic acid comprises a nucleotide sequence as described herein (e.g., TABLE 5, TABLE 6 and TABLE 11). In some embodiments, the guide nucleic acid of the composition comprises a nucleotide sequence of any one of SEQ ID NOS: 166-425 and 436-496 as shown in TABLE 5, TABLE 6 and TABLE 11. In some embodiments, the nucleotide sequence of the guide nucleic acid is at least 65%, at least 70%, at least 75%, at least 80%, at least 85%, at least 90%, at least 95%, or 100% identical to any one of SEQ ID NOS: 166-425 and 436-496 as shown in TABLE 5, TABLE 6 and TABLE 11.

In some embodiments, a guide nucleic acid comprises a nucleotide sequence that is at least 65% identical to any one of SEQ ID NOS: 166-227, 327-425 and 436-498. In some embodiments, a guide nucleic acid comprises a nucleotide sequence that is at least 70% identical to any one of SEQ ID NOS: 166-227, 327-425 and 436-498. In some embodiments, a guide nucleic acid comprises a nucleotide sequence that is at least 75% identical to any one of SEQ ID NOS: 166-227, 327-425 and 436-498. In some embodiments, a guide nucleic acid comprises a nucleotide sequence that is at least 80% identical to any one of SEQ ID NOS: 166-227, 327-425 and 436-498. In some embodiments, a guide nucleic acid comprises a nucleotide sequence that is at least 85% identical to any one of SEQ ID NOS: 166-227, 327-425 and 436-498. In some embodiments, a guide nucleic acid comprises a nucleotide sequence that is at least 90% identical to any one of SEQ ID NOS: 166-227, 327-425 and 436-498. In some embodiments, a guide nucleic acid comprises a nucleotide sequence that is at least 95% identical to any one of SEQ ID NOS: 166-227, 327-425 and 436-498. In some embodiments, a guide nucleic acid comprises a nucleotide sequence that is at least 97% identical to any one of SEQ ID NOS: 166-227, 327-425 and 436-498. In some embodiments, a guide nucleic acid comprises a nucleotide sequence that is at least 99% identical to any one of SEQ ID NOS: 166-227, 327-425 and 436-498. In some embodiments, a guide nucleic acid comprises a nucleotide sequence that is identical to any one of SEQ ID NOS: 166-227, 327-425 and 436-498.

### V. Modifications

Polypeptides (e.g., effector proteins) and nucleic acids (e.g., guide nucleic acids) described herein can be further modified as described throughout and as further described herein.

Examples are modifications of interest that do not alter primary sequence, including chemical derivatization of polypeptides, e.g., acylation, acetylation, carboxylation, amidation, etc. Also included are modifications of glycosylation, e.g., those made by modifying the glycosylation patterns of a polypeptide during its synthesis and processing or in further processing steps; e.g., by exposing the polypeptide to enzymes which affect glycosylation, such as mammalian glycosylating or deglycosylating enzymes. Also embraced are sequences that have phosphorylated amino acid residues, e.g., phosphotyrosine, phosphoserine, or phosphothreonine.

Modifications disclosed herein can also include modification of described polypeptides and/or guide nucleic acids through any suitable method, such as molecular biological techniques and/or synthetic chemistry, to improve their resistance to proteolytic degradation, to change the target sequence specificity, to optimize solubility properties, to alter protein activity (e.g., transcription modulatory activity, enzymatic activity, etc.) or to render them more suitable. Analogs of such polypeptides include those containing residues other than naturally occurring L-amino acids, e.g., D-amino acids or non-naturally occurring synthetic amino acids. D-amino acids may be substituted for some or all of the amino acid residues. Modifications can also include modifications with non-naturally occurring unnatural amino acids. The particular sequence and the manner of preparation will be determined by convenience, economics, purity required, and the like.

Modifications can further include the introduction of various groups to polypeptides and/or guide nucleic acids described herein. For example, groups can be introduced during synthesis or during expression of a polypeptide (e.g., an effector protein), which allow for linking to other molecules or to a surface. Thus, e.g., cysteines can be used to make thioethers, histidines for linking to a metal ion complex, carboxyl groups for forming amides or esters, amino groups for forming amides, and the like.

Modifications can further include modification of nucleic acids described herein (e.g., guide nucleic acids) to provide the nucleic acid with a new or enhanced feature, such as improved stability. Such modifications of a nucleic acid include a base modification, a backbone modification, a sugar modification, or combinations thereof, of one or more nucleotides, nucleosides, or nucleobases in a nucleic acid.

In some embodiments, nucleic acids (e.g., engineered guide nucleic acids) described herein comprise one or more modifications comprising: 2′O-methyl modified nucleotides, 2′ Fluoro modified nucleotides; locked nucleic acid (LNA) modified nucleotides; peptide nucleic acid (PNA) modified nucleotides; nucleotides with phosphorothioate linkages; a 5′ cap (e.g., a 7-methylguanylate cap (m7G)), phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkyl phosphoramidates, phosphorodiamidates, thionophosphor amidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage; phosphorothioate and/or heteroatom internucleoside linkages, such as —CH₂—NH—O—CH₂—, —CH₂—N(CH₃)—O—CH₂— (known as a methylene (methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester internucleotide linkage is represented as —O—P(═O)(OH)—O—CH₂—); morpholino linkages (formed in part from the sugar portion of a nucleoside); morpholino backbones; phosphorodiamidate or other non-phosphodiester internucleoside linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; other backbone modifications having mixed N, O, S and CH₂ component parts; and combinations thereof.

### VI. Vectors and Multiplexed Expression Vectors

In some embodiments, compositions and systems provided herein comprise a vector system, wherein the vector system comprises one or more vectors. When a vector is described herein, such a vector can be used as a vehicle to introduce one or more molecules of interest into a host cell. A molecule of interest can comprise a polypeptide (e.g., an effector protein), an engineered guide or a component thereof (e.g., crRNA, tracrRNA sequence, or sgRNA), a donor nucleic acid, a nucleic acid encoding a polypeptide, a nucleic acid encoding an engineered guide or a component thereof. The vector may be part of a vector system, wherein a vector system comprises a library of vectors each encoding one or more component of a composition or system described herein. In some embodiments, components described herein (e.g., an effector protein, a guide nucleic acid, and/or a target nucleic acid) are encoded by the same vector. In some embodiments, components described herein (e.g., an effector protein, a guide nucleic acid, and/or a target nucleic acid) are each encoded by different vectors of the system. For example, vector systems described herein can comprise one or more vectors comprising a polypeptide (e.g., an effector protein), an engineered guide (e.g., crRNA, tracrRNA sequence, or sgRNA), or encoding for, or a nucleic acid or nucleic acids encoding a polypeptide, engineered guide, a donor nucleic acid, or any combination thereof.

In some embodiments, compositions and systems provided herein comprise a vector system comprising a polypeptide (e.g., an effector protein) described herein. In some embodiments, compositions and systems provided herein comprise a vector system comprising a guide nucleic acid (e.g., crRNA, tracrRNA sequence, or sgRNA) described herein. In some embodiments, compositions and systems provided herein comprise a vector system comprising a donor nucleic acid described herein.

In some embodiments, compositions and systems provided herein comprise a vector system encoding a polypeptide (e.g., an effector protein) described herein. In some embodiments, compositions and systems provided herein comprise a vector system encoding a guide nucleic acid (e.g., crRNA, tracrRNA sequence, or sgRNA) described herein. In some embodiments, compositions and systems provided herein comprise a multi-vector system encoding an effector protein and a guide nucleic acid described herein, wherein the guide nucleic acid and the effector protein are encoded by the same or different vectors. In some embodiments, the guide nucleic acid and the effector protein are encoded by different vectors of the system. In some embodiments, a nucleic acid encoding a polypeptide (e.g., an effector protein) comprises an expression vector. In some embodiments, a nucleic acid encoding a polypeptide is a messenger RNA. In some embodiments, an expression vector comprises or encodes an engineered guide nucleic acid. In some cases, the expression vector encodes the crRNA or sgRNA.

In some embodiments, a vector may encode one or more effector proteins. In some embodiments, the one or more effector proteins comprise at least two effector proteins. In some embodiments, the at least two effector protein are the same. In some embodiments, the at least two effector proteins are different from each other. In some embodiments, the nucleotide sequence is operably linked to a promoter that is operable in a target cell, such as a eukaryotic cell. In some embodiments, a vector may encode 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 effector proteins. In some embodiments, a vector can encode one or more effector proteins comprising an amino acid sequence of any one of SEQ ID NOs: 1-78 and 499.

In some embodiments, a vector may encode one or more guide nucleic acids. In some embodiments, the one or more guide nucleic acids comprise at least two guide nucleic acids. In some embodiments, the at least two guide nucleic acids are the same. In some embodiments, the at least two guide nucleic acids are different from each other. In some embodiments, a vector may encode 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 different guide nucleic acids as described herein. In some embodiments, a vector can encode one or more guide nucleic acids comprising a nucleotide sequence of any one of SEQ ID NOs: 123-227, 327-425 and 436-498. In some embodiments, a vector can encode one or more guide nucleic acids comprising a nucleotide sequence with at least 75%, 80%, 85%, 90%, 95% or 98% sequence identity to any one of SEQ ID NOs: 123-227, 327-425 and 436-498.

In some embodiments, a vector comprises one or more donor nucleic acids as described herein. In some embodiments, the one or more donor nucleic acids comprise at least two donor nucleic acids. In some embodiments, the at least two donor nucleic acids are the same. In some embodiments, the at least two donor nucleic acids are different from each other. In some embodiments, the vector comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more donor nucleic acids.

In some embodiments, a vector can comprise or encode one or more regulatory elements. Regulatory elements can refer to transcriptional and translational control sequences, such as promoters, enhancers, polyadenylation signals, terminators, protein degradation signals, and the like, that provide for and/or regulate transcription of a non-coding sequence or a coding sequence and/or regulate translation of an encoded polypeptide. In some embodiments, a vector can comprise or encode for one or more additional elements, such as, replication origins, antibiotic resistance (or a nucleic acid encoding the same), a tag (or a nucleic acid encoding the same), selectable markers, and the like.

Vectors described herein can encode a promoter—a regulatory region on a nucleic acid, such as a DNA sequence, capable of initiating transcription of a downstream (3′ direction) coding or non-coding sequence. As used herein, a promoter can be bound at its 3′ terminus to a nucleic acid the expression or transcription of which is desired, and extends upstream (5′ direction) to include bases or elements necessary to initiate transcription or induce expression, which could be measured at a detectable level. A promoter can comprise a nucleotide sequence, referred to herein as a “promoter sequence”. A promoter sequence can include a transcription initiation site, and one or more protein binding domains responsible for the binding of transcription machinery, such as RNA polymerase. When eukaryotic promoters are used, such promoters can contain “TATA” boxes and “CAT” boxes. Various promoters, including inducible promoters, may be used to drive expression, i.e., transcriptional activation, of the nucleic acid of interest. Accordingly, in some embodiments, the nucleic acid of interest can be operably linked to a promoter.

Promotors can be any suitable type of promoter envisioned for the compositions, systems, and methods described herein. Examples include constitutively active promoters (e.g., CMV promoter), inducible promoters (e.g., heat shock promoter, tetracycline-regulated promoter, steroid-regulated promoter, metal-regulated promoter, estrogen receptor-regulated promoter, etc.), spatially restricted and/or temporally restricted promoters (e.g., a tissue specific promoter, a cell type specific promoter, etc.), etc. Suitable promoters include, but are not limited to: SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6), an enhanced U6 promoter, and a human H1 promoter (H1). By transcriptional activation, it is intended that transcription will be increased above basal levels in the target cell by 10-fold, by 100-fold, or by 1000-fold, or more. In addition, vectors used for providing a nucleic acid encoding an engineered guide nucleic acid and/or an effector protein to a cell may include nucleic acid sequences that encode for selectable markers in the target cells, so as to identify cells that have taken up the engineered guide nucleic acid and/or an effector protein.

In general, plasmids and vectors described herein comprise at least one promoter. In some embodiments, the length of the promoter is less than about 500, less than about 400, or less than about 300 linked nucleotides. In some embodiments, the length of the promoter is at least 100 linked nucleotides. In some embodiments, the promoters are constitutive promoters. In other embodiments, the promoters are inducible promoters. In some embodiments, the promoter is an inducible promoter that only drives expression of its corresponding gene when a signal is present, e.g., a hormone, a small molecule, a peptide. Non-limiting examples of inducible promoters are the T7 RNA polymerase promoter, the T3 RNA polymerase promoter, the Isopropyl-beta-D-thiogalactopyranoside (IPTG)-regulated promoter, a lactose induced promoter, a heat shock promoter, a tetracycline-regulated promoter (tetracycline-inducible or tetracycline-repressible), a steroid regulated promoter, a metal-regulated promoter, and an estrogen receptor-regulated promoter. In some embodiments, the promoter is an activation-inducible promoter, such as a CD69 promoter, as described further in Kulemzin et al., (2019), BMC Med Genomics, 12:44. In additional embodiments, the promoters are prokaryotic promoters (e.g., drive expression of a gene in a prokaryotic cell). In some embodiments, the promoters are eukaryotic promoters, (e.g., drive expression of a gene in a eukaryotic cell). Exemplary promoters include, but are not limited to, CMV, 7SK, EF1a, RPBSA, hPGK, EFS, SV40, PGK1, Ube, human beta actin, CAG, TRE, UAS, Ac5, polyhedron, CaMKIIa, GAL1-10, TEF1, GDS, ADH1, CaMV35S, Ubi, H1, U6, CaMV35S, MNDU3, MSCV and HSV TK promoter. In some embodiments, the promoter is CMV. In some embodiments, the promoter is EF1a. In some embodiments, the promoter is ubiquitin. In some embodiments, vectors are bicistronic or polycistronic vector (e.g., having or involving two or more loci responsible for generating a protein) having an internal ribosome entry site (IRES) is for translation initiation in a cap-independent manner.

In some embodiments, a vector described herein is a delivery vector. In some examples, the delivery vector may be a eukaryotic vector, a prokaryotic vector (e.g., a bacterial vector) a viral vector, or any combination thereof. In some embodiments, the delivery vehicle may be a non-viral vector. In some embodiments, the delivery vehicle may be a plasmid. In some embodiments, the plasmid comprises DNA. In some embodiments, the plasmid comprises RNA. In some examples, the plasmid comprises circular double-stranded DNA. In some examples, the plasmid may be linear. In some examples, the plasmid comprises one or more genes of interest and one or more regulatory elements. In some examples, the plasmid comprises a bacterial backbone containing an origin of replication and an antibiotic resistance gene or other selectable marker for plasmid amplification in bacteria. In some examples, the plasmid may be a minicircle plasmid. In some examples, the plasmid contains one or more genes that provide a selective marker to induce a target cell to retain the plasmid. In some examples, the plasmid may be formulated for delivery through injection by a needle carrying syringe. In some examples, the plasmid may be formulated for delivery via electroporation. In some examples, the plasmids may be engineered through synthetic or other suitable means known in the art. For example, in some cases, the genetic elements may be assembled by restriction digest of the desired genetic sequence from a donor plasmid or organism to produce ends of the DNA which may then be readily ligated to another genetic sequence.

In some embodiments, vectors comprise an enhancer. Enhancers are nucleotide sequences that have the effect of enhancing promoter activity. In some embodiments, enhancers augment transcription regardless of the orientation of their sequence. In some embodiments, enhancers activate transcription from a distance of several kilo basepairs. Furthermore, enhancers are located optionally upstream or downstream of a gene region to be transcribed, and/or located within the gene, to activate the transcription. Exemplary enhancers include, but are not limited to, WPRE; CMV enhancers; the R-U5′ segment in LTR of HTLV-I (Mol. Cell. Biol., Vol. 8(1), p. 466-472, 1988); SV40 enhancer; the intron sequence between exons 2 and 3 of rabbit β-globin (Proc. Natl. Acad. Sci. USA., Vol. 78(3), p. 1527-31, 1981); and the genome region of human growth hormone (J Immunol., Vol. 155(3), p. 1286-95, 1995).

In some embodiments, an effector protein (or a nucleic acid encoding same) and/or an engineered guide nucleic acid (or a nucleic acid encoding same) are co-administered with a donor nucleic acid. Co-administration can be contact with a target nucleic acid, administered to a cell, such as a host cell, or administered as method of nucleic acid detection, editing, and/or treatment as described herein, in a single vehicle, such as a single expression vector. In certain embodiments, an effector protein (or a nucleic acid encoding same) and/or an engineered guide nucleic acid (or a nucleic acid encoding same) are not co-administered with donor nucleic acid in a single vehicle. In certain embodiments, an effector protein (or a nucleic acid encoding same), an engineered guide nucleic acid (or a nucleic acid encoding same), and/or donor nucleic acid are administered in one or more or two or more vehicles, such as one or more, or two or more expression vectors.

### Administration of a Non-Viral Vector

In some embodiments, the vector is a non-viral vector, and a physical method or a chemical method is employed for delivery into the somatic cell. Exemplary physical methods include electroporation, gene gun, sonoporation, magnetofection, or hydrodynamic delivery. Exemplary chemical methods include delivery of the recombinant polynucleotide via liposomes such as, cationic lipids or neutral lipids; dendrimers; nanoparticles; or cell-penetrating peptides.

In some embodiments, a vector is administered as part of a method of nucleic acid detection, editing, and/or treatment as described herein. In some embodiments, a vector is administered in a single vehicle, such as a single expression vector. In some embodiments, at least two of the three components, a nucleic acid encoding one or more effector proteins, one or more donor nucleic acids, and one or more guide nucleic acids or a nucleic acid encoding the one or more guide nucleic acid, are provided in the single expression vector. In some embodiments, components, such as a guide nucleic acid and an effector protein, are encoded by the same vector. In some embodiments, an effector protein (or a nucleic acid encoding same) and/or an engineered guide nucleic acid (or a nucleic acid that, when transcribed, produces same) are not co-administered with donor nucleic acid in a single vehicle. In some embodiments, an effector protein (or a nucleic acid encoding same), an engineered guide nucleic acid (or a nucleic acid that, when transcribed, produces same), and/or donor nucleic acid are administered in one or more or two or more vehicles, such as one or more, or two or more expression vectors.

In some embodiments, a vector may be part of a vector system. In some embodiments, the vector system comprises a library of vectors each encoding one or more components of a composition or system described herein. In some embodiments, a vector system is administered as part of a method of nucleic acid detection, editing, and/or treatment as described herein, wherein at least two vectors are co-administered. In some embodiments, the at least two vectors comprise different components. In some embodiments, the at least two vectors comprise the same component having different sequences. In some embodiments, at least one of the three components, a nucleic acid encoding one or more effector proteins, one or more donor nucleic acids, and one or more guide nucleic acids or a nucleic acid encoding the one or more guide nucleic acids, or a variant thereof is provided in a different vector. In some embodiments, the nucleic acid encoding the effector protein, and a guide nucleic acid or a nucleic acid encoding the guide nucleic acid are provided in different vectors. In some embodiments, the donor nucleic acid is encoded by a different vector than the vector encoding the effector protein and the guide nucleic acid.

### Lipid Particles and Non-Viral Vectors

In some embodiments, compositions and systems provided herein comprise a lipid particle. In some embodiments, a lipid particle is a lipid nanoparticle (LNP). In some embodiments, a lipid or a lipid nanoparticle can encapsulate an expression vector. In some embodiments, a lipid or a lipid nanoparticle can encapsulate the effector protein, the guide nucleic acid, the nucleic acid encoding the effector protein and/or the DNA molecule encoding the guide nucleic acid. LNPs are a non-viral delivery system for gene therapy. LNPs are effective for delivery of nucleic acids. Beneficial properties of LNP include ease of manufacture, low cytotoxicity and immunogenicity, high efficiency of nucleic acid encapsulation and cell transfection, multi-dosing capabilities and flexibility of design (Kulkarni et al., (2018) Nucleic Acid Therapeutics, 28(3):146-157). In some embodiments, a method can comprise contacting a cell with an expression vector. In some embodiments, contacting can comprise electroporation, lipofection, or lipid nanoparticle (LNP) delivery of an expression vector. In some embodiments, a nucleic acid expression vector is a non-viral vector. In some embodiments, compositions and methods comprise a lipid, polymer, nanoparticle, or a combination thereof, or use thereof, to introduce a Cas protein, guide nucleic acid, donor template or any combination thereof to a cell. Non-limiting examples of lipids and polymers are cationic polymers, cationic lipids, or bio-responsive polymers. In some embodiments, the bio-responsive polymer exploits chemical-physical properties of the endosomal environment (e.g., pH) to preferentially release the genetic material in the intracellular space.

In some embodiments, a LNP comprises an outer shell and an inner core. In some embodiments, the outer shell comprises lipids. In some embodiments, the lipids comprise modified lipids. In some embodiments, the modified lipids comprise pegylated lipids. In some embodiments, the lipids comprise one or more of cationic lipids, anionic lipids, ionizable lipids, and non-ionic lipids. In some embodiments, the LNP comprises one or more of N1,N3,N5-tris(3-(didodecylamino)propyl)benzene-1,3,5-tricarboxamide (TT3), 2-dioleoyl-sn-glycero-3-phosphoethanolamine (DOPE), 1-palmitoyl-2-oleoylsn-glycero-3-phosphoethanolamine (POPE), 1,2-distearoyl-sn-glycero-3-phosphocholine (DSPC), cholesterol (Chol), 1,2-dimyristoyl-sn-glycerol, and methoxypolyethylene glycol (DMG-PEChooo), derivatives, analogs, or variants thereof. In some embodiments, the LNP has a negative net overall charge prior to complexation with one or more of a guide nucleic acid, a nucleic acid encoding the one or more guide nucleic acid, a nucleic acid encoding the effector protein, and/or a donor nucleic acid. In some embodiments, the inner core is a hydrophobic core. In some embodiments, the one or more of a guide nucleic acid, the one or more nucleic acid encoding the one or more guide nucleic acid, one or more nucleic acid encoding one or more effector protein, and/or the one or more donor nucleic acid forms a complex with one or more of the cationic lipids and the ionizable lipids. In some embodiments, the nucleic acid encoding the effector protein or the nucleic acid encoding the guide nucleic acid is self-replicating.

In some embodiments, a LNP comprises one or more of cationic lipids, ionizable lipids, and modified versions thereof. In some embodiments, the ionizable lipid comprises TT3 or a derivative thereof. Accordingly, in some embodiments, the LNP comprises one or more of TT3 and pegylated TT3. The publication WO2016187531 is hereby incorporated by reference in its entirety, which describes representative LNP formulations in Table 2 and Table 3, and representative methods of delivering LNP formulations in Example 7.

In some embodiments, a LNP comprises a lipid composition targeting to a specific organ. In some embodiments, the lipid composition comprises lipids having a specific alkyl chain length that controls accumulation of the LNP in the specific organ (e.g., liver or spleen). In some embodiments, the lipid composition comprises a biomimetic lipid that controls accumulation of the LNP in the specific organ (e.g., brain). In some embodiments, the lipid composition comprises lipid derivatives (e.g., cholesterol derivatives) that controls accumulation of the LNP in a specific cell (e.g., liver endothelial cells, Kupffer cells, hepatocytes).

### Delivery of Viral Vectors

An expression vector can be a viral vector. In some embodiments, a viral vector comprises a nucleic acid to be delivered into a host cell via a recombinantly produced virus or viral particle. The nucleic acid may be single-stranded or double stranded, linear or circular, segmented or non-segmented. The nucleic acid may comprise DNA, RNA, or a combination thereof. In some embodiments, the expression vector is an adeno-associated viral vector. There are a variety of viral vectors that are associated with various types of viruses, including but not limited to retroviruses (e.g., lentiviruses and γ-retroviruses), adenoviruses, arenaviruses, alphaviruses, adeno-associated viruses (AAVs), baculoviruses, vaccinia viruses, herpes simplex viruses and poxviruses. The virus may be a lentivirus. The virus may be an adenovirus. The virus may be a non-replicating virus. The virus may be an adeno-associated virus (AAV). The viral vector may be a retroviral vector. Retroviral vectors may include gamma-retroviral vectors such as vectors derived from the Moloney Murine Leukemia Virus (MoMLV, MMLV, MuLV, or MLV) or the Murine Stem cell Virus (MSCV) genome. Retroviral vectors may include lentiviral vectors such as those derived from the human immunodeficiency virus (HIV) genome. In some embodiments, the viral vector is a chimeric viral vector, comprising viral portions from two or more viruses. In some embodiments, the viral vector is a recombinant viral vector. In some embodiments, the viral vector is a chimeric viral vector. In some embodiments, the chimeric viral vector comprises viral portions from two or more viruses. In some embodiments, the viral vector corresponds to a virus of a specific serotype.

In some embodiments, the viral vector is an AAV. The AAV may be any AAV known in the art. In some embodiments, a viral particle that delivers a viral vector described herein is an AAV. In some embodiments, the viral vector corresponds to a virus of a specific serotype. In some examples, the serotype is selected from an AAV1 serotype, an AAV2 serotype, AAV3 serotype, an AAV4 serotype, AAV5 serotype, an AAV6 serotype, AAV7 serotype, an AAV8 serotype, an AAV9 serotype, an AAV10 serotype, an AAV-rh10 serotype, an AAV 11 serotype, and an AAV12 serotype. In some embodiments the AAV vector is a recombinant vector, a hybrid AAV vector, a chimeric AAV vector, a self-complementary AAV (scAAV) vector, a single-stranded AAV or any combination thereof scAAV genomes are generally known in the art and contain both DNA strands which can anneal together to form double-stranded DNA.

In some embodiments, the AAV vector may be a chimeric AAV vector. In some embodiments, the chimeric AAV vector comprises an exogenous amino acid or an amino acid substitution, or capsid proteins from two or more serotypes. In some examples, a chimeric AAV vector may be genetically engineered to increase transduction efficiency, selectivity, or a combination thereof.

In some embodiments, the viral vector is a replication-defective viral vector, comprising an insertion of a therapeutic gene inserted in genes essential to the lytic cycle, preventing the virus from replicating and exerting cytotoxic effects. A viral vector provided herein can be derived from or based on any such virus. Often the viral vectors provided herein are an adeno-associated viral vector (AAV vector). Generally, an AAV vector has two inverted terminal repeats (ITRs). According, in some embodiments, the viral vector provided herein comprises two inverted terminal repeats of AAV. The DNA sequence in between the ITRs of an AAV vector provided herein may be referred to herein as the sequence encoding the genome editing tools. These genome editing tools can include, but are not limited to, an effector protein, effector protein modifications (e.g., nuclear localization signal (NLS), polyA tail), guide nucleic acid(s), respective promoter(s), and a donor nucleic acid, or combinations thereof. In general, viral vectors provided herein comprise at least one promotor or a combination of promoters driving expression or transcription of one or more genome editing tools described herein. In some embodiments, the coding region of the AAV vector forms an intramolecular double-stranded DNA template thereby generating an AAV vector that is a self-complementary AAV (scAAV) vector. In general, the sequence encoding the genome editing tools of an scAAV vector has a length of about 2 kb to about 3 kb. The scAAV vector can comprise nucleotide sequences encoding an effector protein, providing guide nucleic acids described herein, and a donor nucleic acid described herein. In some embodiments, the AAV vector provided herein is a self-inactivating AAV vector. In some embodiments, an AAV vector provided herein comprises a modification, such as an insertion, deletion, chemical alteration, or synthetic modification, relative to a wild-type AAV vector.

In some embodiments, a fusion effector protein as described herein is inserted into a vector. In some embodiments, the vector comprises one or more promoters, enhancers, ribosome binding sites, RNA splice sites, polyadenylation sites, a replication origin, and/or transcriptional terminator sequences.

### Producing AAV Delivery Vectors

In some embodiments, methods of producing delivery vectors herein comprise packaging a nucleic acid encoding an effector protein and a guide nucleic acid, or a combination thereof, into an AAV vector. In some embodiments, methods of producing the delivery vector comprises, (a) contacting a cell with at least one nucleic acid encoding: (i) a guide nucleic acid; (ii) a Replication (Rep) gene; and (iii) a Capsid (Cap) gene that encodes an AAV capsid protein; (b) expressing the AAV capsid protein in the cell; (c) assembling an AAV particle; and (d) packaging a Cas effector encoding nucleic acid into the AAV particle, thereby generating an AAV delivery vector. In some embodiments, promoters, stuffer sequences, and any combination thereof may be packaged in the AAV vector. In some examples, the AAV vector can package 1, 2, 3, 4, or 5 guide nucleic acids or copies thereof. In some embodiments, the AAV vector comprises inverted terminal repeats, e.g., a 5′ inverted terminal repeat and a 3′ inverted terminal repeat. In some embodiments, the AAV vector comprises a mutated inverted terminal repeat that lacks a terminal resolution site.

In some embodiments, a hybrid AAV vector is produced by transcapsidation, e.g., packaging an inverted terminal repeat (ITR) from a first serotype into a capsid of a second serotype, wherein the first and second serotypes may be not the same. In some examples, the Rep gene and ITR from a first AAV serotype (e.g., AAV2) may be used in a capsid from a second AAV serotype (e.g., AAV9), wherein the first and second AAV serotypes may be not the same. As a non-limiting example, a hybrid AAV serotype comprising the AAV2 ITRs and AAV9 capsid protein may be indicated AAV2/9. In some examples, the hybrid AAV delivery vector comprises an AAV2/1, AAV2/2, AAV 2/4, AAV2/5, AAV2/8, or AAV2/9 vector.

### Producing AAV Particles

The AAV particles described herein can be referred to as recombinant AAV (rAAV). Often, rAAV particles are generated by transfecting AAV producing cells with an AAV-containing plasmid carrying the sequence encoding the genome editing tools, a plasmid that carries viral encoding regions, i.e., Rep and Cap gene regions; and a plasmid that provides the helper genes such as E1A, E1B, E2A, E40RF6 and VA. In some embodiments, the AAV producing cells are mammalian cells. In some embodiments, host cells for rAAV viral particle production are mammalian cells. In some embodiments, a mammalian cell for rAAV viral particle production is a COS cell, a HEK293T cell, a HeLa cell, a KB cell, a derivative thereof, or a combination thereof. In some embodiments, rAAV virus particles can be produced in the mammalian cell culture system by providing the rAAV plasmid to the mammalian cell. In some embodiments, producing rAAV virus particles in a mammalian cell can comprise transfecting vectors that express the rep protein, the capsid protein, and the gene-of-interest expression construct flanked by the ITR sequence on the 5′ and 3′ ends. Methods of such processes are provided in, for example, Naso et al., BioDrugs, 2017 August; 31(4):317-334 and Benskey et al., (2019), Methods Mol Biol., 1937:3-26, each of which is incorporated by reference in their entireties.

In some embodiments, rAAV is produced in a non-mammalian cell. In some embodiments, rAAV is produced in an insect cell. In some embodiments, an insect cell for producing rAAV viral particles comprises a Sf9 cell. In some embodiments, production of rAAV virus particles in insect cells can comprise baculovirus. In some embodiments, production of rAAV virus particles in insect cells can comprise infecting the insect cells with three recombinant baculoviruses, one carrying the cap gene, one carrying the rep gene, and one carrying the gene-of-interest expression construct enclosed by an ITR on both the 5′ and 3′ end. In some embodiments, rAAV virus particles are produced by the One Bac system. In some embodiments, rAAV virus particles can be produced by the Two Bac system. In some embodiments, in the Two Bac system, the rep gene and the cap gene of the AAV is integrated into one baculovirus virus genome, and the ITR sequence and the gene-of-interest expression construct is integrated into another baculovirus virus genome. In some embodiments, in the One Bac system, an insect cell line that expresses both the rep protein and the capsid protein is established and infected with a baculovirus virus integrated with the ITR sequence and the gene-of-interest expression construct. Details of such processes are provided in, for example, Smith et. al., (1983), Mol. Cell. Biol., 3(12):2156-65; Urabe et al., (2002), Hum. Gene. Ther., 1; 13(16):1935-43; and Benskey et al., (2019), Methods Mol Biol., 1937:3-26, each of which is incorporated by reference in its entirety.

In some embodiments, the viral particle that delivers the viral vector described herein is an AAV. AAVs are characterized by their serotype. Non-limiting examples of AAV serotypes are AAV1, AAV2, AAV3, AAV4, AAV5, AAV6, AAV7, AAV8, AAV9, AAV10, AAV11, AAV12, scAAV, AAV-rh10, chimeric or hybrid AAV, or any combination, derivative, or variant thereof.

### VII. Certain Target Nucleic Acids

Disclosed herein are compositions, systems, and methods for modifying and detecting target nucleic acids. In some embodiments, the target nucleic acid is a double stranded nucleic acid. In some embodiments, the target nucleic acid is a single stranded nucleic acid. In some embodiments, the target nucleic acid is a double stranded nucleic acid that is prepared into single stranded nucleic acids before or upon contacting a reagent or sample. In some embodiments, the target nucleic acid comprises DNA. In some embodiments, the target nucleic acid comprises RNA. The target nucleic acids include but are not limited to mRNA, rRNA, tRNA, non-coding RNA, long non-coding RNA, and microRNA (miRNA). In some embodiments, the target nucleic acid is complementary DNA (cDNA) synthesized from a single-stranded RNA template in a reaction catalyzed by a reverse transcriptase. In some cases, the target nucleic acid is single-stranded RNA (ssRNA) or mRNA. In some embodiments, the target nucleic acid is a double stranded nucleic acid comprising a target strand and a non-target strand, wherein the target strand comprises a target sequence. In some embodiments, where a target strand comprises a target sequence, at least a portion of the engineered guide nucleic acid is complementary to the target sequence on the target strand. In some embodiments, where the target nucleic acid is a double stranded nucleic acid comprising a target strand and a non-target strand, and wherein the target strand comprises a target sequence, at least a portion of the engineered guide nucleic acid is complementary to the target sequence on the target strand. In some embodiments, a target nucleic acid comprises a PAM as described herein that is located on the non-target strand. Such a PAM described herein, in some embodiments, is adjacent (e.g., within 1, 2, 3, 4 or 5 nucleotides) to the 3′ end of the target sequence on the non-target strand of the double stranded DNA molecule. In certain embodiments, such a PAM described herein is directly adjacent to the 3′ end of a target sequence on the non-target strand of the double stranded DNA molecule. In some embodiments, the PAM sequence 5′ end to the target sequence. In some embodiments, the PAM sequence is directly 5′ end to the target sequence. In some embodiments, the target nucleic acid as described in the methods herein does not initially comprise a PAM sequence. However, any target nucleic acid of interest may be generated using the methods described herein to comprise a PAM sequence, and thus be a PAM target nucleic acid. A PAM target nucleic acid, as used herein, refers to a target nucleic acid that has been amplified to insert a PAM sequence that is recognized by an effector protein system.

In some embodiments, target nucleic acids comprise a mutation. In some embodiments, a composition, system or method described herein can be used to modify a target nucleic acid comprising a mutation such that the mutation is modified to be a wild-type nucleotide or nucleotide sequence. In some embodiments, a composition, system or method described herein can be used to detect a target nucleic acid comprising a mutation. The mutation may be a mutation of 1, 2, 3, 4, 5, 6, 7, 8, 9, 10, 11, 12, 13, 14, 15, 16, 17, 18, 19, 20 or more nucleotides. The mutation can be a deletion of about 5, about 10, about 15, about 20, about 25, about 30, about 35, about 40, about 45, about 50, about 55, about 60, about 65, about 70, about 75, about 80, about 85, about 90, about 95, about 100, about 200, about 300, about 400, about 500, about 600, about 700, about 800, about 900, or about 1000 nucleotides. The mutation can be a deletion of 1 to 5, 5 to 10, 10 to 15, 15 to 20, 20 to 25, 25 to 30, 30 to 35, 35 to 40, 40 to 45, 45 to 50, 50 to 55, 55 to 60, 60 to 65, 65 to 70, 70 to 75, 75 to 80, 80 to 85, 85 to 90, 90 to 95, 95 to 100, 100 to 200, 200 to 300, 300 to 400, 400 to 500, 500 to 600, 600 to 700, 700 to 800, 800 to 900, 900 to 1000, 1 to 50, 1 to 100, 25 to 50, 25 to 100, 50 to 100, 100 to 500, 100 to 1000, or 500 to 1000 nucleotides. The mutation can be in the open reading frame of a target nucleic acid that results in the insertion of at least one amino acid in a protein encoded by the target nucleic acid. The mutation can also be in the open reading frame of a target nucleic acid that results in the deletion of at least one amino acid in a protein encoded by the target nucleic acid. The mutation can be in the open reading frame of a target nucleic acid that results in the substitution of at least one amino acid in a protein encoded by the target nucleic acid. A mutation that results in the deletion, insertion, or substitution of one or more amino acids of a protein encoded by the target nucleic acid can result in misfolding of the protein. The mutation can result in a premature stop codon. The mutation can result in a truncation of the protein.

In some embodiments, at least a portion of a guide nucleic acid of a composition described herein hybridizes to a region of the target nucleic acid comprising the mutation. In some embodiments, at least a portion of a guide nucleic acid of a composition described herein hybridizes to a region of the target nucleic acid that is within 10 nucleotides, within 50 nucleotides, within 100 nucleotides, or within 200 nucleotides of the mutation. The mutation may be located in a non-coding region or a coding region of a gene.

In some embodiments, compositions, systems, and methods described herein comprise a target nucleic acid may be responsible for a disease, contain a mutation (e.g., single strand polymorphism, point mutation, insertion, or deletion), be contained in an amplicon, or be uniquely identifiable from the surrounding nucleic acids (e.g., contain a unique sequence of nucleotides). In some embodiments, the target nucleic acid has undergone a modification (e.g., an editing) after contacting with an RNP. In some embodiments, the editing is a change in the sequence of the target nucleic acid. In some embodiments, the change comprises an insertion, deletion, or substitution of one or more nucleotides compared to the target nucleic acid that has not undergone any modification.

In some embodiments, the mutation is an autosomal dominant mutation. In some embodiments, the mutation is a dominant negative mutation. In some embodiments, the mutation is a loss of function mutation. In some embodiments, the mutation is a single nucleotide polymorphism (SNP). In some embodiments, the SNP is associated with a phenotype of the sample or a phenotype of the organism from which the sample was taken. The SNP, in some cases, is associated with altered phenotype from wild type phenotype. The SNP may be a synonymous substitution or a nonsynonymous substitution. The nonsynonymous substitution may be a missense substitution, or a nonsense point mutation. The synonymous substitution may be a silent substitution. The mutation may be a deletion of one or more nucleotides. Often, the single nucleotide mutation, SNP, or deletion is associated with a disease such as cancer or a genetic disorder. The mutation, such as a single nucleotide mutation, a SNP, or a deletion, may be encoded in the sequence of a target nucleic acid from the germline of an organism or may be encoded in a target nucleic acid from a diseased cell, such as a cancer cell.

In some embodiments, the target nucleic acid comprises a mutation associated with a disease. In some examples, a mutation associated with a disease refers to a mutation whose presence in a subject indicates that the subject is susceptible to, or suffers from, a disease, disorder, condition, or syndrome. In some examples, a mutation associated with a disease refers to a mutation which causes, contributes to the development of, or indicates the existence of the disease, disorder, condition, or syndrome. A mutation associated with a disease can also refer to any mutation which generates transcription or translation products at an abnormal level, or in an abnormal form, in cells affected by a disease relative to a control without the disease. In some embodiments, the mutation causes the disease.

In some embodiments, the target nucleic acid is from a gene with a mutation associated with a genetic disorder, from a gene whose overexpression is associated with a genetic disorder, from a gene associated with abnormal cellular growth resulting in a genetic disorder, or from a gene associated with abnormal cellular metabolism resulting in a genetic disorder. Non-limiting examples of diseases associated with genetic mutations are recited in TABLE 7.

The disease may comprise, at least in part, a cancer, an inherited disorder, an ophthalmological disorder, a neurological disorder, a neurodegenerative disease, a blood disorder, a metabolic disorder, a genetic disorder, an infection, or a combination thereof. In some embodiments, the cancer is a solid cancer (i.e., a tumor). In some embodiments, the cancer is a blood cell cancer, including a leukemia or lymphoma. In some embodiments, the cancer is colon cancer, rectal cancer, renal-cell carcinoma, liver cancer, bladder cancer, cancer of the kidney or ureter, lung cancer, cancer of the small intestine, esophageal cancer, melanoma, bone cancer, pancreatic cancer, skin cancer, brain cancer (e.g., glioblastoma), cancer of the head or neck, melanoma, uterine cancer, ovarian cancer, breast cancer, testicular cancer, cervical cancer, stomach cancer, Hodgkin's Disease, non-Hodgkin's lymphoma, thyroid cancer. The cancer may be a leukemia, such as, by way of non-limiting example, acute myeloid (or myelogenous) leukemia (AML), chronic myeloid (or myelogenous) leukemia (CML), acute lymphocytic (or lymphoblastic) leukemia (ALL), and chronic lymphocytic leukemia (CLL).

In some embodiments, the target nucleic acid comprises a portion of a gene comprising a mutation associated with cancer, a gene whose overexpression is associated with cancer, a tumor suppressor gene, an oncogene, a checkpoint inhibitor gene, a gene associated with cellular growth, a gene associated with cellular metabolism, or a gene associated with cell cycle. Sometimes, the target nucleic acid encodes a cancer biomarker, such as a prostate cancer biomarker or non-small cell lung cancer. In some cases, the assay may be used to detect “hotspots” in target nucleic acids that may be predictive of lung cancer. In some cases, the target nucleic acid comprises a portion of a nucleic acid that is associated with a hemorrhagic fever.

In some cases, the target nucleic acid is a portion of a nucleic acid from a genomic locus, any DNA amplicon of, a reverse transcribed mRNA, or a cDNA from a locus of at least one of the genes recited in TABLE 8.

In some cases, the target nucleic acid is a portion of a nucleic acid from a genomic locus, any DNA amplicon of, a reverse transcribed mRNA, or a cDNA from a locus of at least one of: ABL, ACE, AF4/HRX, AKT-2, ALK, ALK/NPM, AML1, AML1/MTG8, APC, ATM, AXIN2, AXL, BAP1, BARD1, BCL-2, BCL-3, BCL-6, BCR/ABL, BLM, BMPR1A, BRCA1, BRCA2, BRIP1, c-MYC, CASR, CCR5, CDC73, CDH1, CDK4, CDKN1B, CDKN1C, CDKN2A, CEBPA, CHEK2, CREBBP, CTNNA1, DBL, DEK/CAN, DICER1, DIS3L2, E2A/PBX1, EGFR, ENL/HRX, EPCAM, ERG/TLS, ERBB, ERBB-2, ETS-1, EWS/FLI-1, FH, FKRP, FLCN, FMS, FOS, FPS, GATA2, GCG, GLI, GPC3, GPGSP, GREM1, HER2/neu, HOX11, HOXB13, HRAS, HST, IL-3, INT-2, JAK1, JUN, KIT, KS3, K-SAM, LBC, LCK, LMO1, LMO2, L-MYC, LYL-1, LYT-10, LYT-10/Ca1, MAS, MAX MDM-2, MEN1, MET, MITF, MLH1, MLL, MOS, MSH1, MSH2, MSH3, MSH6, MTG8/AML1, MUTYH, MYB, MYH1/CBFB, NBN, NEU, NF1, NF2, N-MYC, NTHL1, OST, PALB2, PAX-5, PBX11E2A, PCDC1, PDGFRA, PHOX2B, PIM-1, PMS2, POLD1, POLE, POT1, PPARG, PRAD-1, PRKAR1A, PTCH1, PTEN, RAD50, RAD51C, RAD51D, RAF, PAR PML, RAS-H, RAS-K, RAS-N, RB1, RECQL4, REL/NRG, RET, RHOM1, RHOM2, ROS, RUNX1, SDHA, SDHAF, SDHAF2, SDHB, SDHC, SDHD, SET/CAN, SIS, SKI, SMAD4, SMARCA4, SMARCB, SMARCE1, SRC, STKI1, SUFU, TAL1, TAL2, TAN-1, TIAM1, TERC, TERT, TIMP3, TMEM127, TNF, TP53, TRAC, TSC1, TSC2, TRK, VHL, WRN, and WT1. Non-limiting examples of oncogenes are KRAS, NRAS, BRAF, MYC, CTNNB1, and EGFR. In some embodiments, the oncogene is a gene that encodes a cyclin dependent kinase (CDK). Non-limiting examples of CDKs are Cdk1, Cdk4, Cdk5, Cdk7, Cdk8, Cdk9, Cdk11 and CDK20. Non-limiting examples of tumor suppressor genes are TP53, RB1, and PTEN. Any region of the aforementioned gene loci may be probed for a mutation or deletion using the compositions and methods disclosed herein. For example, in the EGFR gene locus, the compositions and methods for detection disclosed herein may be used to detect a single nucleotide polymorphism or a deletion.

In some cases, the target nucleic acid comprises a portion of a nucleic acid from a genomic locus, any DNA amplicon of, a reverse transcribed mRNA, or a cDNA from a locus of at least one of: TRAC, B2M, PD1, PCSK9, DNMT1, HPRT1, RPL32P3, CCR5, FANCF, GRIN2B, EMX1, AAVS1, ALKBH5, CLTA, CDK11, CTNNB1, AXIN1, LRP6, TBK1, BAP1, TLE3, PPM1A, BCL2L2, SUFU, RICTOR, VPS35, TOP1, SIRT1, PTEN, MMD, PAQR8, H2AX, POU5F1, OCT4, SYS1, ARFRP1, TSPAN14, EMC2, EMC3, SEL1L, DERL2, UBE2G2, UBE2J1, and HRD1.

In some embodiments, the method for treating a disease comprises modifying at least one gene associated with the disease or modifying expression of the at least one gene such that the disease is treated. In some embodiments, the disease is Alzheimer's disease and the gene is selected from APP, BACE-1, PSD95, MAPT, PSEN1, PSEN2, and APOEe4. In some embodiments, the disease is congenital muscular dystrophy 1A (MDC1A) and the gene is LAMA1 or LAMA2. In some embodiments, the disease is Ullrich Congenital Muscular Dystrophy (UCMD) and the gene is selected from COL6A1, COL6A2 and COL6A3. In some embodiments, the disease is Limb Girdle Muscular Dystrophies (LGMD1B, LGMD2A, LGMD2B) and the gene is selected from LMNA, DYSF, and CAPN3. In some embodiments, the disease is Nemaline Myopathy and the gene is selected from ACTA1, NEB, TPM2, TPM3, TNNT1, TNNT3, TNNI2 and LMOD3.

In some embodiments, the disease is Parkinson's disease and the gene is selected from SNCA, GDNF, and LRRK2. In some embodiments, the disease comprises Centronuclear myopathy and the gene is DNM2. In some embodiments, the disease is Huntington's disease and the gene is HTT. In some embodiments, the disease is Alpha-1 antitrypsin deficiency (AATD) and the gene is SERPINA1. In some embodiments, the disease is amyotrophic lateral sclerosis (ALS) and the gene is selected from SOD1, FUS, C90RF72, ATXN2, TARDBP, and CHCHD10. In some embodiments, the disease comprises Alexander Disease and the gene is GFAP. In some embodiments, the disease comprises anaplastic large cell lymphoma and the gene is CD30. In some embodiments, the disease comprises Angelman Syndrome and the gene is UBE3A. In some embodiments, the disease comprises Calcific Aortic Stenosis and the gene is Apo(a). In some embodiments, the disease comprises CD3Z-associated primary T-cell immunodeficiency and the gene is CD3Z or CD247. In some embodiments, the disease comprises CD18 deficiency and the gene is ITGB2. In some embodiments, the disease comprises CD40L deficiency and the gene is CD40L. In some embodiments, the disease comprises CNS trauma and the gene is VEGF. In some embodiments, the disease comprises coronary heart disease and the gene is selected from FGA, FGB, and FGG. In some embodiments, the disease comprises MECP2 Duplication syndrome and Rett syndrome and the gene is MECP2. In some embodiments, the disease comprises a bleeding disorder (coagulation) and the gene is FXI. In some embodiments, the disease comprises fragile X syndrome and the gene is FMR1. In some embodiments, the disease comprises Fuchs Corneal Dystrophy and the gene is selected from ZEB1, SLC4A11, and LOXHD1. In some embodiments, the disease comprises GM2-Gangliosidoses (e.g., Tay Sachs Disease, Sandhoff disease) and the gene is selected from HEXA and HEXB. In some embodiments, the disease comprises Hearing loss disorders and the gene is DFNA36. In some embodiments, the disease is Pompe disease, including infantile onset Pompe Disease (IOPD) and late onset Pompe Disease (LOPD) and the gene is GAA. In some embodiments, the disease is Retinitis pigmentosa and the gene is selected from PDE6B, RHO, RP1, RP2, RPGR, PRPH2, IMPDH1, PRPF31, CRB1, PRPF8, TULP1, CA4, HPRPF3, ABCA4, EYS, CERKL, FSCN2, TOPORS, SNRNP200, PRCD, NR2E3, MERTK, USH2A, PROM1, KLHL7, CNGB1, TTC8, ARL6, DHDDS, BEST1, LRAT, SPARA7, CRX, CLRN1, RPE65, and WDR19. In some embodiments, the disease comprises Leber Congenital Amaurosis Type 10 and the gene is CEP290. In some embodiments, the disease is cardiovascular disease and/or lipodystrophies and the gene is selected from ABCG5, ABCG8, AGT, ANGPTL3, APOCIII, APOA1, APOL1, ARH, CDKN2B, CFB, CXCL12, FXI, FXII, GATA-4, MIA3, MKL2, MTHFD1L, MYH7, NKX2-5, NOTCH1, PKK, PCSK9, PSRC1, SMAD3, and TTR. In some embodiments, the disease is cardiovascular disease and/or lipodystrophies and the gene is ANGPTL3. In some embodiments, the disease is cardiovascular disease and/or lipodystrophies and the gene is PCSK9. In some embodiments, the disease is cardiovascular disease and/or lipodystrophies and the gene is TTR. In some embodiments, the disease is severe hypertriglyceridemia (SHTG) and the gene is APOCIII or ANGPTL4. In some embodiments, the disease comprises acromegaly and the gene is GHR. In some embodiments, the disease comprises acute myeloid leukemia and the gene is CD22. In some embodiments, the disease is diabetes and the gene is GCGR. In some embodiments, the disease is NAFLD/NASH and the gene is selected from HSD17B13, PSD3, GPAM, CIDEB, DGAT2 and PNPLA3. In some embodiments, the disease is NASH/cirrhosis and the gene is MARC1. In some embodiments, the disease is cancer and the gene is selected from STAT3, YAP1, FOXP3, AR (Prostate cancer), and IRF4 (multiple myeloma). In some embodiments, the disease is cystic fibrosis and the gene is CFTR. In some embodiments, the disease is Duchenne Muscular Dystrophy and the gene is DMD. In some embodiments, the disease is ornithine transcarbamylase deficiency (OTCD) and the gene is OTC. In some embodiments, the disease is congenital adrenal hyperplasia (CAH) and the gene is CYP21A2. In some embodiments, the disease is atherosclerotic cardiovascular disease (ASCVD) and the gene is LPA. In some embodiments, the disease is hepatitis B virus infection (CHB) and the gene is HBV covalently closed circular DNA (cccDNA). In some embodiments, the disease is citrullinemia type I and the gene is ASS1. In some embodiments, the disease is citrullinemia type I and the gene is SLC25A13. In some embodiments, the disease is citrullinemia type I and the gene is ASS1. In some embodiments, the disease is arginase-1 deficiency and the gene is ARG1. In some embodiments, the disease is carbamoyl phosphate synthetase I deficiency and the gene is CPS1. In some embodiments, the disease is argininosuccinic aciduria and the gene is ASL. In some embodiments, the disease comprises angioedema and the gene is PKK. In some embodiments, the disease comprises thalassemia and the gene is TMPRSS6. In some embodiments, the disease comprises achondroplasia and the gene is FGFR3. In some embodiments, the disease comprises Cri du chat syndrome and the gene is selected from CTNND2. In some embodiments, the disease comprises sickle cell anemia and the gene is Beta globin gene. In some embodiments, the disease comprises Alagille Syndrome and the gene is selected from JAG1 and NOTCH2. In some embodiments, the disease comprises Charcot Marie Tooth Disease and the gene is selected from PMP22 and MFN2. In some embodiments, the disease comprises Crouzon syndrome and the gene is selected from FGFR2, FGFR3, and FGFR3. In some embodiments, the disease comprises Dravet Syndrome and the gene is selected from SCN1A and SCN2A. In some embodiments, the disease comprises Emery-Dreifuss syndrome and the gene is selected from EMD, LMNA, SYNE1, SYNE2, FHL1, and TMEM43. In some embodiments, the disease comprises Factor V Leiden Thrombophilia and the gene is F5. In some embodiments, the disease is fabry disease and the gene is GLA. In some embodiments, the disease is facioscapulohumeral muscular dystrophy (FSHD) and the gene is FSHD1. In some embodiments, the disease comprises Fanconi anemia and the gene is selected from FANCA, FANCB, FANCC, FANCD1, FANCD2, FANCE, FANCF, FANCG, FANCI, FANCJ, FANCL, FANCM, FANCN, FANCP, FANCS, RAD51C, and XPF. In some embodiments, the disease comprises Familial Creutzfeld-Jakob Disease and the gene is PRNP. In some embodiments, the disease comprises Familial Mediterranean Fever and the gene is MEFV. In some embodiments, the disease comprises Friedreich's ataxia and the gene is FXN. In some embodiments, the disease comprises Gaucher disease and the gene is GBA. In some embodiments, the disease comprises human papilloma virus (HPV) infection and the gene is HPV E7. In some embodiments, the disease comprises Hemochromatosis and the gene is HFE, optionally comprising a C282Y mutation. In some embodiments, the disease comprises Hemophilia A and the gene is FVIII. In some embodiments, the disease is hereditary angioedema and the gene is SERPING1 or KLKB1. In some embodiments, the disease comprises histiocytosis and the gene is CD1. In some embodiments, the disease comprises immunodeficiency 17 and the gene is CD3D. In some embodiments, the disease comprises immunodeficiency 13 and the gene is CD4. In some embodiments, the disease comprises Common Variable Immunodeficiency and the gene is selected from CD19 and CD81. In some embodiments, the disease comprises Joubert syndrome and the gene is selected from INPP5E, TMEM216, AHI1, NPHP1, CEP290, TMEM67, RPGRIP1L, ARL13B, CC2D2A, OFD1, TMEM138, TCTN3, ZNF423, and AMRC9. In some embodiments, the disease comprises leukocyte adhesion deficiency and the gene is CD18. In some embodiments, the disease comprises Li-Fraumeni syndrome and the gene is TP53. In some embodiments, the disease comprises lymphoproliferative syndrome and the gene is CD27. In some embodiments, the disease comprises Lynch syndrome and the gene is selected from MSH2, MLH1, MSH6, PMS2, PMS1, TGFBR2, and MLH3. In some embodiments, the disease comprises mantle cell lymphoma and the gene is CD5. In some embodiments, the disease comprises Marfan syndrome and the gene is FBN1. In some embodiments, the disease comprises mastocytosis and the gene is CD2. In some embodiments, the disease comprises methylmalonic acidemia and the gene is selected from MMAA, MMAB, and MUT. In some embodiments, the disease is mycosis fungoides and the gene is CD7. In some embodiments, the disease is myotonic dystrophy and the gene is selected from CNBP and DMPK. In some embodiments, the disease comprises neurofibromatosis and the gene is selected from NF1, and NF2. In some embodiments, the disease comprises osteogenesis imperfecta and the gene is selected from COL1A1, COL1A2, and IFITM5. In some embodiments, the disease is non-small cell lung cancer and the gene is selected from KRAS, EGFR, ALK, METex14, BRAF V600E, ROS1, RET, and NTRK. In some embodiments, the disease comprises Peutz-Jeghers syndrome and the gene is STKI1. In some embodiments, the disease comprises polycystic kidney disease and the gene is selected from PKD1 and PKD2. In some embodiments, the disease comprises Severe Combined Immune Deficiency and the gene is selected from IL7R, RAG1, JAK3. In some embodiments, the disease comprises PRKAG2 cardiac syndrome and the gene is PRKAG2. In some embodiments, the disease comprises Spinocerebellar ataxia and the gene is selected from ATXN1, ATXN2, ATXN3, PLEKHG4, SPTBN2, CACNA1A, ATXN7, ATXN8OS, ATXN10, TTBK2, PPP2R2B, KCNC3, PRKCG, ITPR1, TBP, KCND3, and FGF14. In some embodiments, the disease is thrombophilia due to antithrombin III deficiency and the gene is SERPINC1. In some embodiments the disease is spinal muscular atrophy and the gene is SMN1. In some embodiments, the disease comprises Usher Syndrome and the gene is selected from MYO7A, USH1C, CDH23, PCDH15, USH1G, USH2A, GPR98, DFNB31, and CLRN1. In some embodiments, the disease comprises von Willebrand disease and the gene is VWF. In some embodiments, the disease comprises Waardenburg syndrome and the gene is selected from PAX3, MITF, WS2B, WS2C, SNAI2, EDNRB, EDN3, and SOX10. In some embodiments, the disease comprises Wiskott-Aldrich Syndrome and the gene is WAS. In some embodiments, the disease comprises von Hippel-Lindau disease and the gene is VHL. In some embodiments, the disease comprises Wilson disease and the gene is ATP7B. In some embodiments, the disease comprises Zellweger syndrome and the gene is selected from PEX1, PEX2, PEX3, PEX5, PEX6, PEX10, PEX12, PEX13, PEX14, PEX16, PEX19, and PEX26. In some embodiments, the disease comprises infantile myofibromatosis and the gene is CD34. In some embodiments, the disease comprises platelet glycoprotein IV deficiency and the gene is CD36. In some embodiments, the disease comprises immunodeficiency with hyper-IgM type 3 and the gene is CD40. In some embodiments, the disease comprises hemolytic uremic syndrome and the gene is CD46. In some embodiments, the disease comprises complement hyperactivation, angiopathic thrombosis, or protein-losing enteropathy and the gene is CD55. In some embodiments, the disease comprises hemolytic anemia and the gene is CD59. In some embodiments, the disease comprises calcification of joints and arteries and the gene is CD73. In some embodiments, the disease comprises immunoglobulin alpha deficiency and the gene is CD79A. In some embodiments, the disease comprises C syndrome and the gene is CD96. In some embodiments, the disease comprises hairy cell leukemia and the gene is CD123. In some embodiments, the disease comprises histiocytic sarcoma and the gene is CD163. In some embodiments, the disease comprises autosomal dominant deafness and the gene is CD164. In some embodiments, the disease comprises immunodeficiency 25 and the gene is CD247. In some embodiments, the disease comprises methymalonic acidemia due to transcobalamin receptor defect and the gene is CD320.

In some embodiments, treatment of a disease comprises administration of a gene therapy. In some embodiments, a gene therapy comprises use of a vector to introduce a functional gene or transgene. In some embodiments, vectors comprise nonviral vectors, including cationic polymers, cationic lipids, or bio-responsive polymers. In some embodiments, the bio-responsive polymer exploits chemical-physical properties of the endosomal environment (e.g., pH) to preferentially release the genetic material in the intracellular space. In some embodiments, vectors comprise viral vectors, including retroviruses, adenoviruses, adeno-associated viruses, and herpes simplex viruses. In some embodiments, the vector comprises a replication-defective viral vector, comprising an insertion of a therapeutic gene inserted in genes essential to the lytic cycle, preventing the virus from replicating and exerting cytotoxic effects. Methods of gene therapy are described in more detail in Ingusci et al., “Gene Therapy Tools for Brain Diseases”, Front. Pharmacol. 10:724 (2019) which is hereby incorporated by reference in its entirety.

The target nucleic acid may be from any organism, including, but not limited to, a bacterium, a virus, a parasite, a protozoon, a fungus, a mammal, a plant, and an insect. As another non-limiting example, the target nucleic acid may be responsible for a disease, contain a mutation (e.g., single strand polymorphism, point mutation, insertion, or deletion), be contained in an amplicon, or be uniquely identifiable from the surrounding nucleic acids (e.g., contain a unique sequence of nucleotides). In some embodiments, the target nucleic acid is from a bacteria. In some embodiments, the bacteria is Acholeplasma laidlawii, Brucella abortus, Chlamydia psittaci, Chlamydia trachomatis, Cryptococcus neoformans, Escherichia coli, Legionella pneumophila, Lyme disease spirochetes, methicillin-resistant Staphylococcus aureus, Mycobacterium leprae, Mycobacterium tuberculosis, Mycoplasma arginini, Mycoplasma arthritidis, Mycoplasma genitalium, Mycoplasma hyorhinis, Mycoplasma orale, Mycoplasma pneumoniae, Mycoplasma salivarium, Neisseria gonorrhoeae, Neisseria meningitidis, Pneumococcus, Pseudomonas aeruginosa, Streptococcus agalactiae, Streptococcus pyogenes, Treponema pallidum, or any combination thereof.

In some embodiments, the target nucleic acid is from a virus. In some embodiments, the virus is adenovirus, blue tongue virus, chikungunya, coronavirus (e.g. SARS-CoV-2), cytomegalovirus, Dengue virus, Ebola, Epstein-Barr virus, feline leukemia virus, Hemophilus influenzae B, Hepatitis Virus A, Hepatitis Virus B, Hepatitis Virus C, herpes simplex virus I, herpes simplex virus II, human papillomavirus (HPV) including HPV16 and HPV18, human serum parvo-like virus, human T-cell leukemia viruses, immunodeficiency virus (e.g. HIV), influenza virus, lymphocytic choriomeningitis virus, measles virus, mouse mammary tumor virus, mumps virus, murine leukemia virus, polio virus, rabies virus, Reovirus, respiratory syncytial virus (RSV), rubella virus, Sendai virus, simian virus 40, Sindbis virus, varicella-zoster virus, vesicular stomatitis virus, wart virus, West Nile virus, yellow fever virus, or any combination thereof. In some cases, the target nucleic acid comprises a portion of a nucleic acid that is associated with a hemorrhagic fever.

In some embodiments, the target nucleic acid is from a parasite. In some embodiments, the parasite is a helminth, an annelid, a platyhelminth, a nematode, or a thorny-headed worms. In some embodiments, the parasite is Babesia bovis, Echinococcus granulosus, Eimeria tenella, Leishmania tropica, Mesocestoides corti, Onchocerca volvulus, Plasmodium falciparum, Plasmodium vivax, Schistosoma japonicum, Schistosoma mansoni, Schistosoma spp., Taenia hydatigena, Taenia ovis, Taenia saginata, Theileria parva, Toxoplasma gondii, Toxoplasma spp., Trichinella spiralis, Trichomonas vaginalis, Trypanosoma brucei, Trypanosoma cruzi, Trypanosoma rangeli, Trypanosoma rhodesiense, Balantidium coli, Entamoeba histolytica, Giardia spp., Isospora spp., Trichomonas spp., or any combination thereof.

In some embodiments, the target nucleic acid comprises a nucleic acid sequence from a pathogen responsible for a disease. Non-limiting examples of pathogens are bacteria, a virus and a fungus. The target nucleic acid, in some embodiments, is a portion of a nucleic acid from a sexually transmitted infection or a contagious disease. In some embodiments, the target nucleic acid is a portion of a nucleic acid from a genomic locus, or any DNA amplicon, such as a reverse transcribed mRNA or a cDNA from a gene locus, a transcribed mRNA, or a reverse transcribed cDNA from a gene locus in at least one of: human immunodeficiency virus (HIV), human papillomavirus (HPV), chlamydia, gonorrhea, syphilis, trichomoniasis, sexually transmitted infection, malaria, Dengue fever, Ebola, chikungunya, and leishmaniasis. Pathogens include viruses, fungi, helminths, protozoa, malarial parasites, Plasmodium parasites, Toxoplasma parasites, and Schistosoma parasites. Helminths include roundworms, heartworms, and phytophagous nematodes, flukes, Acanthocephala, and tapeworms. Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, P. vivax, Trypanosoma cruzi and Toxoplasma gondii. Fungal pathogens include, but are not limited to Cryptococcus neoformans, Histoplasma capsulatum, Coccidioides immitis, Blastomyces dermatitidis, Chlamydia trachomatis, and Candida albicans. Pathogenic viruses include but are not limited to coronavirus (e.g., SARS-CoV-2); immunodeficiency virus (e.g., HIV); influenza virus; dengue; West Nile virus; herpes virus; yellow fever virus; Hepatitis Virus C; Hepatitis Virus A; Hepatitis Virus B; papillomavirus; and the like. Pathogens include, e.g., HIV virus, Mycobacterium tuberculosis, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus (RSV), M. genitalium, T. vaginalis, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M arginini, Acholeplasma laidlawii, M. salivarium and M. pneumoniae. In some embodiments, the target sequence is a portion of a nucleic acid from a genomic locus, a transcribed mRNA, or a reverse transcribed cDNA from a gene locus of bacterium or other agents responsible for a disease in the sample comprising a mutation that confers resistance to a treatment, such as a single nucleotide mutation that confers resistance to antibiotic treatment.

In some embodiments, the target nucleic acid comprises a nucleic acid sequence of a virus, a bacterium, or other pathogen responsible for a disease in a plant (e.g., a crop). Methods and compositions of the disclosure may be used to treat or detect a disease in a plant. For example, the methods of the disclosure may be used to target a viral nucleic acid sequence in a plant. An effector protein of the disclosure may cleave the viral nucleic acid. In some embodiments, the target nucleic acid comprises a nucleic acid sequence of a virus or a bacterium or other agents (e.g., any pathogen) responsible for a disease in the plant (e.g., a crop). In some embodiments, the target nucleic acid comprises RNA. The target nucleic acid, in some embodiments, is a portion of a nucleic acid from a virus or a bacterium or other agents responsible for a disease in the plant (e.g., a crop). In some embodiments, the target nucleic acid is a portion of a nucleic acid from a genomic locus, or any NA amplicon, such as a reverse transcribed mRNA or a cDNA from a gene locus, a transcribed mRNA, or a reverse transcribed cDNA from a gene locus in at a virus or a bacterium or other agents (e.g., any pathogen) responsible for a disease in the plant (e.g., a crop). A virus infecting the plant may be an RNA virus. A virus infecting the plant may be a DNA virus. Non-limiting examples of viruses that may be targeted with the disclosure include Tobacco mosaic virus (TMV), Tomato spotted wilt virus (TSWV), Cucumber mosaic virus (CMV), Potato virus Y (PVY), Cauliflower mosaic virus (CaMV) (RT virus), Plum pox virus (PPV), Brome mosaic virus (BMV) and Potato virus X (PVX).

In some embodiments, the target nucleic acid is any one of: a naturally occurring eukaryotic sequence, a naturally occurring prokaryotic sequence, a naturally occurring viral sequence, a naturally occurring bacterial sequence, a naturally occurring fungal sequence, an engineered eukaryotic sequence, an engineered prokaryotic sequence, an engineered viral sequence, an engineered bacterial sequence, an engineered fungal sequence, a fragment of a naturally occurring sequence, a fragment of an engineered sequence, and combinations thereof.

In some embodiments, the target nucleic acid is isolated from any one of: a naturally occurring cell, a eukaryotic cell, a prokaryotic cell, a plant cell, a fungal cell, an animal cell, cell of an invertebrate, a fly cell, a cell of a vertebrate, a mammalian cell, a primate cell, a non-human primate cell, a human cell, a living cell, a non-living cell, a modified cell, a derived cell, and a non-naturally occurring cell.

Nucleic acids, such as DNA and pre-mRNA, described herein can contain at least one intron and at least one exon, wherein as read in the 5′ to the 3′ direction of a nucleic acid strand, the 3′ end of an intron can be adjacent to the 5′ end of an exon, and wherein said intron and exon correspond for transcription purposes. If a nucleic acid strand contains more than one intron and exon, the 5′ end of the second intron is adjacent to the 3′ end of the first exon, and 5′ end of the second exon is adjacent to the 3′ end of the second intron. The junction between an intron and an exon can be referred to herein as a splice junction, wherein a 5′ splice site (SS) can refer to the +1/+2 position at the 5′ end of intron and a 3′SS can refer to the last two positions at the 3′ end of an intron. Alternatively, a 5′ SS can refer to the 5′ end of an exon and a 3′SS can refer to the 3′ end of an exon. In some embodiments, nucleic acids can contain one or more elements that act as a signal during transcription, splicing, and/or translation. In some embodiments, signaling elements include a 5′SS, a 3′SS, a premature stop codon, U1 and/or U2 binding sequences, and cis acting elements such as branch site (BS), polypyridine tract (PYT), exonic and intronic splicing enhancers (ESEs and ISEs) or silencers (ESSs and ISSs). In some embodiments, nucleic acids may also comprise a untranslated region (UTR), such as a 5′ UTR or a 3′ UTR. In some embodiments, the start of an exon or intron is referred to interchangeably herein as the 5′ end of an exon or intron, respectively. Likewise, in some embodiments, the end of an exon or intron is referred to interchangeably herein as the 3′ end of an exon or intron, respectively.

In some embodiments, at least a portion of at least one target sequence is within about 1, about 5 or more, about 10 or more, about 15 or more, about 20 or more, about 25 or more, about 30 or more, about 35 or more, about 40 or more, about 45 or more, about 50 or more, about 55 or more, about 60 or more, about 65 or more, about 70 or more, about 75 or more, about 80 or more, about 85 or more, about 90 or more, about 95 or more, about 100 or more, about 105 or more, about 110 or more, about 115 or more, about 120 or more, about 125 or more, about 130 or more, about 135 or more, about 140 or more, about 145 or more, or about 150 to about 300 nucleotides adjacent to: the 5′ end of an exon; the 3′ end of an exon; the 5′ end of an intron; the 3′ end of an intron; one or more signaling element comprising a 5′SS, a 3′SS, a premature stop codon, U1 binding sequence, U2 binding sequence, a BS, a PYT, ESE, an ISE, an ESS, an ISS; a 5′ UTR; a 3′ UTR; more than one of the foregoing, or any combination thereof. In some embodiments, the target nucleic acid comprises a target locus. In some embodiments, the target nucleic acid comprises more than one target loci. In some embodiments, the target nucleic acid comprises two target loci. Accordingly, in some embodiments, the target nucleic acid can comprise one or more target sequences.

In some embodiments, compositions, systems, and methods described herein comprise an edited target nucleic acid which can describe a target nucleic acid wherein the target nucleic acid has undergone a change, for example, after contact with an effector protein. In some embodiments, the editing is an alteration in the sequence of the target nucleic acid. In some embodiments, the edited target nucleic acid comprises an insertion, deletion, or replacement of one or more nucleotides compared to the unedited target nucleic acid. In some embodiments, the editing is a mutation.

### Mutations

In some embodiments, target nucleic acids described herein comprise a mutation. In some embodiments, a composition, system or method described herein can be used to edit a target nucleic acid comprising a mutation such that the mutation is edited to be the wild-type nucleotide or nucleotide sequence. In some embodiments, a composition, system or method described herein can be used to detect a target nucleic acid comprising a mutation. A mutation may result in the insertion of at least one amino acid in a protein encoded by the target nucleic acid. A mutation may result in the deletion of at least one amino acid in a protein encoded by the target nucleic acid. A mutation may result in the substitution of at least one amino acid in a protein encoded by the target nucleic acid. A mutation that results in the deletion, insertion, or substitution of one or more amino acids of a protein encoded by the target nucleic acid may result in misfolding of a protein encoded by the target nucleic acid. A mutation may result in a premature stop codon, thereby resulting in a truncation of the encoded protein.

Non-limiting examples of mutations are insertion-deletion (indel), a point mutation, single nucleotide polymorphism (SNP), a chromosomal mutation, a copy number mutation or variation, and frameshift mutations. In some embodiments, an indel mutation is an insertion or deletion of one or more nucleotides. In some embodiments, a point mutation comprises a substitution, insertion, or deletion. In some embodiments, a frameshift mutation occurs when the number of nucleotides in the insertion/deletion is not divisible by three, and it occurs in a protein coding region. In some embodiments, a chromosomal mutation can comprise an inversion, a deletion, a duplication, or a translocation of one or more nucleotides. In some embodiments, a copy number variation can comprise a gene amplification or an expanding trinucleotide repeat. In some embodiments, an SNP is associated with a phenotype of the sample or a phenotype of the organism from which the sample was taken. In some embodiments, an SNP is associated with altered phenotype from wild type phenotype. In some embodiments, the SNP is a synonymous substitution or a nonsynonymous substitution. In some embodiments, the nonsynonymous substitution is a missense substitution or a nonsense point mutation. In some embodiments, the synonymous substitution is a silent substitution.

In some embodiments, a target nucleic acid described herein comprises a mutation of one or more nucleotides. In some embodiments, the one or more nucleotides comprises 1, 2, 3, 4, 5, 6, 7, 8, 9, 10, 11, 12, 13, 14, 15, 16, 17, 18, 19, 20 or more nucleotides. In some embodiments, the mutation comprises a deletion, insertion, and/or substitution of about 5, about 10, about 15, about 20, about 25, about 30, about 35, about 40, about 45, about 50, about 55, about 60, about 65, about 70, about 75, about 80, about 85, about 90, about 95, about 100, about 200, about 300, about 400, about 500, about 600, about 700, about 800, about 900, or about 1000 nucleotides. In some embodiments, the mutation comprises a deletion, insertion, and/or substitution of 1 to 5, 5 to 10, 10 to 15, 15 to 20, 20 to 25, 25 to 30, 30 to 35, 35 to 40, 40 to 45, 45 to 50, 50 to 55, 55 to 60, 60 to 65, 65 to 70, 70 to 75, 75 to 80, 80 to 85, 85 to 90, 90 to 95, 95 to 100, 100 to 200, 200 to 300, 300 to 400, 400 to 500, 500 to 600, 600 to 700, 700 to 800, 800 to 900, 900 to 1000, 1 to 50, 1 to 100, 25 to 50, 25 to 100, 50 to 100, 100 to 500, 100 to 1000, or 500 to 1000 nucleotides. The mutation may be located in a non-coding region or a coding region of a gene, wherein the gene is a target nucleic acid. A mutation may be in an open reading frame of a target nucleic acid. In some embodiments, guide nucleic acids described herein hybridize to a portion of the target nucleic acid comprising or adjacent to the mutation.

In some embodiments, target nucleic acids comprise a mutation, wherein the mutation is a SNP. In some embodiments, the single nucleotide mutation or SNP is associated with a phenotype of the sample or a phenotype of the organism from which the sample was taken. In some embodiments, the SNP is associated with altered phenotype from wild type phenotype. In some embodiments, a single nucleotide mutation, SNP, or deletion described herein is associated with a disease, such as a genetic disease. In some embodiments, the SNP is a synonymous substitution or a nonsynonymous substitution. In some embodiments, the nonsynonymous substitution is a missense substitution or a nonsense point mutation. In some embodiments, the synonymous substitution is a silent substitution. In some embodiments, the mutation is a deletion of one or more nucleotides. In some embodiments, the single nucleotide mutation, SNP, or deletion is associated with a disease such as a genetic disorder. In some embodiments, the mutation, such as a single nucleotide mutation, a SNP, or a deletion, may be encoded in the sequence of a target nucleic acid from the germline of an organism or may be encoded in a target nucleic acid from a diseased cell.

In some embodiments, the mutation is associated with a disease, such as a genetic disorder. In some embodiments, the mutation may be encoded in the sequence of a target nucleic acid from the germline of an organism or may be encoded in a target nucleic acid from a diseased cell. In some embodiments, a target nucleic acid described herein comprises a mutation associated with a disease. In some examples, a mutation associated with a disease refers to a mutation whose presence in a subject indicates that the subject is susceptible to or suffers from, a disease, disorder, condition, or syndrome. In some examples, a mutation associated with a disease refers to a mutation which causes, contributes to the development of, or indicates the existence of the disease, disorder, condition, or syndrome. A mutation associated with a disease may also refer to any mutation which generates transcription or translation products at an abnormal level, or in an abnormal form, in cells affected by a disease relative to a control without the disease. In some embodiments, a mutation associated with a disease, comprises the co-occurrence of a mutation and the phenotype of a disease. The mutation may occur in a gene, wherein transcription or translation products from the gene occur at a significantly abnormal level or in an abnormal form in a cell or subject harboring the mutation as compared to a non-disease control subject not having the mutation. In some embodiments, a target nucleic acid described herein comprises a mutation associated with a disease described herein.

### Detection and Identification of Target Nucleic Acid

In some embodiments, a target nucleic acid is in a cell. In some embodiments, the cell is a single-cell eukaryotic organism; a plant cell an algal cell; a fungal cell; an animal cell; a cell of an invertebrate animal; a cell of a vertebrate animal such as fish, amphibian, reptile, bird, and mammal; or a cell of a mammal such as a human, a non-human primate, an ungulate, a feline, a bovine, an ovine, and a caprine. In some embodiments, the cell is a eukaryotic cell. In some embodiments, the cell is a mammalian cell, a human cell, or a plant cell. In some embodiments, the cell is a human cell. In some embodiments, the human cell is a: muscle cell, liver cell, lung cell, cardiac cell, visceral cell, cardiac muscle cell, smooth muscle cell, cardiomyocyte, nodal cardiac muscle cell, smooth muscle cell, visceral muscle cell, skeletal muscle cell, myocyte, red (or slow) skeletal muscle cell, white (fast) skeletal muscle cell, intermediate skeletal muscle, muscle satellite cell, muscle stem cell, myoblast, muscle progenitor cell, induced pluripotent stem cell (iPS), or a cell derived from an iPS cell, modified to have its gene edited and differentiated into myoblasts, muscle progenitor cells, muscle satellite cells, muscle stem cells, skeletal muscle cells, cardiac muscle cells or smooth muscle cells.

In some embodiments, an effector protein-guide nucleic acid complex may comprise high selectivity for a target sequence. In some embodiments, an RNP comprise a selectivity of at least 200:1, 100:1, 50:1, 20:1, 10:1, or 5:1 for a target nucleic acid over a single nucleotide variant of the target nucleic acid. In some embodiments, an RNP may comprise a selectivity of at least 5:1 for a target nucleic acid over a single nucleotide variant of the target nucleic acid.

By leveraging such effector protein selectivity, some methods described herein may detect a target nucleic acid present in the sample in various concentrations or amounts as a target nucleic acid population. In some embodiments, the method detects at least 2 target nucleic acid populations. In some embodiments, the method detects at least 3, 4, 5, 6, 7, 8, 9, 10, 20, 30, 40, or 50 target nucleic acid populations. In some embodiments, the method detects 3 to 50, 5 to 40, or 10 to 25 target nucleic acid populations. In some embodiments, the method detects at least 2 individual target nucleic acids. In some embodiments, the method detects at least 3, 5, 10, 20, 30, 40, 50, 100, 200, 300, 400, 500, 600, 700, 800, 900, 1000, 2000, 3000, 4000, 5000, 6000, 7000, 8000, 9000, or 10000 individual target nucleic acids. In some embodiments, the method detects 1 to 10,000, 100 to 8000, 400 to 6000, 500 to 5000, 1000 to 4000, or 2000 to 3000 individual target nucleic acids. In some embodiments, the method detects target nucleic acid present at least at one copy per 10 non-target nucleic acids, 10² non-target nucleic acids, 10³ non-target nucleic acids, 10⁴ non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids.

In some embodiments, compositions described herein exhibit indiscriminate trans-cleavage of a nucleic acid (e.g., ssRNA or ssDNA), enabling their use for detection of a nucleic acid (e.g., RNA or DNA, respectively) in samples. In some embodiments, target nucleic acids are generated from many nucleic acid templates (e.g., RNA) in order to achieve cleavage of a reporter (e.g., a FQ reporter) in a device (e.g., a DETECTR platform). Certain effector proteins may be activated by a nucleic acid (e.g., ssDNA or ssRNA), upon which they may exhibit trans-cleavage of the nucleic acid (e.g., ssDNA or ssRNA) and may, thereby, be used to cleave reporter molecules (e.g., ssDNA or ssRNA FQ reporter molecules) in a device (e.g., a DETECTR). These effector proteins may target nucleic acids present in the sample or nucleic acids generated and/or amplified from any number of nucleic acid templates (e.g., RNA). Described herein are reagents comprising a single stranded reporter nucleic acid comprising a detection moiety, wherein the reporter nucleic acid (e.g., a ssDNA-FQ reporter described herein) is capable of being cleaved by the effector protein, upon generation (e.g., cDNA) and amplification of nucleic acids from a nucleic acid template (e.g., ssRNA) using the methods disclosed herein, thereby generating a first detectable signal. While DNA and RNA are used as an exemplary reporter in the foregoing, any suitable reporter may be used.

In some embodiments, a target nucleic acid is an amplified nucleic acid of interest. In some embodiments, the nucleic acid of interest is any nucleic acid disclosed herein or from any sample as disclosed herein. In some embodiments, the nucleic acid of interest is an RNA that is reverse transcribed before amplification. In some embodiments, the nucleic acid of interest is amplified then the amplicons is transcribed into RNA.

In some embodiments, target nucleic acids may activate an effector protein to initiate sequence-independent cleavage of a nucleic acid-based reporter (e.g., a reporter comprising an RNA sequence, or a reporter comprising DNA and RNA). For example, an effector protein of the present disclosure is activated by a target nucleic acid to cleave reporters having an RNA (also referred to herein as an “RNA reporter”). Alternatively, an effector protein of the present disclosure is activated by a target nucleic acid to cleave reporters having an RNA. Alternatively, an effector protein of the present disclosure is activated by a target RNA to cleave reporters having an RNA (also referred to herein as a “RNA reporter”). The RNA reporter may comprise a single-stranded RNA labelled with a detection moiety or may be any RNA reporter as disclosed herein.

Further description of editing or detecting a target nucleic acid in a gene of interest can be found in more detail in Kim et al., “Enhancement of target specificity of CRISPR-Cas12a by using a chimeric DNA-RNA guide”, Nucleic Acids Res. 2020 Sep. 4; 48(15):8601-8616; Wang et al., “Specificity profiling of CRISPR system reveals greatly enhanced off-target gene editing”, Scientific Reports volume 10, Article number: 2269 (2020); Tuladhar et al., “CRISPR-Cas9-based mutagenesis frequently provokes on-target mRNA misregulation”, Nature Communications volume 10, Article number: 4056 (2019); Dong et al., “Genome-Wide Off-Target Analysis in CRISPR-Cas9 Modified Mice and Their Offspring”, G3, Volume 9, Issue 11, 1 Nov. 2019, Pages 3645-3651; Winter et al., “Genome-wide CRISPR screen reveals novel host factors required for Staphylococcus aureus α-hemolysin-mediated toxicity”, Scientific Reports volume 6, Article number: 24242 (2016); and Ma et al., “A CRISPR-Based Screen Identifies Genes Essential for West-Nile-Virus-Induced Cell Death”, Cell Rep. 2015 Jul. 28; 12(4):673-83, which are hereby incorporated by reference in their entirety.

### VIII. Compositions

Disclosed herein are compositions comprising one or more effector proteins described herein or nucleic acids encoding the one or more effector proteins, one or more guide nucleic acids described herein or nucleic acids encoding the one or more guide nucleic acids described herein, or combinations thereof. In some embodiments, one or more of a repeat sequence of the one or more guide nucleic acids are capable of interacting with the one or more of the effector proteins. In some embodiments, spacer sequences of the one or more guide nucleic acids hybridizes with a target sequence of a target nucleic acid. In some embodiments, the compositions comprise one or more donor nucleic acids described herein. In some embodiments, the compositions are capable of editing a target nucleic acid in a cell or a subject. In some embodiments, the compositions are capable of editing a target nucleic acid or the expression thereof in a cell, in a tissue, in an organ, in vitro, in vivo, or ex vivo. In some embodiments, the compositions are capable of editing a target nucleic acid in a sample comprising the target nucleic.

In some embodiments, compositions described herein comprise plasmids described herein, viral vectors described herein, non-viral vectors described herein, or combinations thereof. In some embodiments, compositions described herein comprise the viral vectors. In some embodiments, compositions described herein comprise an AAV. In some embodiments, compositions described herein comprise liposomes (e.g., cationic lipids or neutral lipids), dendrimers, lipid nanoparticle (LNP), or cell-penetrating peptides. In some embodiments, compositions described herein comprise an LNP.

### Pharmaceutical Compositions and Modes of Administration

Disclosed herein, in some aspects, are pharmaceutical compositions for modifying a target nucleic acid in a cell or a subject, comprising any one of the effector proteins, engineered effector proteins, or fusion effector proteins described herein. Also disclosed herein, in some aspects, are pharmaceutical compositions comprising a nucleic acid encoding any one of the effector proteins, engineered effector proteins, or fusion effector proteins described herein. In some embodiments, pharmaceutical compositions comprise a guide nucleic acid. In some embodiments, pharmaceutical compositions comprise a plurality of guide nucleic acids. Pharmaceutical compositions may be used to modify a target nucleic acid or the expression thereof in a cell in vitro, in vivo or ex vivo.

In some embodiments, the pharmaceutically acceptable excipient, carrier or diluent allows the active ingredient to retain biological activity. In some embodiments, the pharmaceutically acceptable excipient, carrier or diluent is non-reactive with the subject's immune system. In some embodiments, the pharmaceutically acceptable excipient, carrier or diluent provides for long-term stabilization of the composition. In some embodiments, the pharmaceutically acceptable excipient, carrier or diluent is provided as a bulking agent in solid formulations that contain potent active ingredients in small amounts. In some embodiments, the pharmaceutically acceptable excipient, carrier or diluent confers a therapeutic enhancement on the active ingredient in the final dosage form. In some embodiments, the pharmaceutically acceptable excipient, carrier or diluent facilitates absorption, reduces viscosity, or enhances solubility. In some embodiments, the pharmaceutically acceptable excipient, carrier or diluent is selected based upon the route of administration, dosage form, active ingredient, other factors, or any combination thereof. In some embodiments, the pharmaceutically acceptable excipient, carrier or diluent can be formulated by well-known conventional methods (see, e.g., Remington's Pharmaceutical Sciences, 18th edition, A. Gennaro, ed., Mack Publishing Co., Easton, Pa., 1990; and Remington, The Science and Practice of Pharmacy 21st Ed. Mack Publishing, 2005).

In some embodiments, pharmaceutical compositions comprise one or more nucleic acids encoding an effector protein, fusion effector protein, fusion partner, a guide nucleic acid, or a combination thereof; and a pharmaceutically acceptable carrier or diluent. The effector protein, fusion effector protein, fusion partner protein, or combination thereof may be any one of those described herein. The one or more nucleic acids may comprise a plasmid. The one or more nucleic acids may comprise a nucleic acid expression vector. The one or more nucleic acids may comprise a viral vector. In some embodiments, the viral vector is a lentiviral vector. In some embodiments, the vector is an adeno-associated viral (AAV) vector. In some embodiments, compositions, including pharmaceutical compositions, comprise a viral vector encoding a fusion effector protein and a guide nucleic acid, wherein at least a portion of the guide nucleic acid binds to the effector protein of the fusion effector protein. In some embodiments, pharmaceutical compositions comprise a virus comprising a viral vector encoding a fusion effector protein, an effector protein, a fusion partner, a guide nucleic acid, or a combination thereof; and a pharmaceutically acceptable carrier or diluent.

Pharmaceutical compositions described herein may comprise a salt. In some embodiments, the salt is a sodium salt. In some embodiments, the salt is a potassium salt. In some embodiments, the salt is a magnesium salt. In some embodiments, the salt is NaCl. In some embodiments, the salt is KNO₃. In some embodiments, the salt is Mg²+SO₄²⁻.

Non-limiting examples of pharmaceutically acceptable carriers and diluents suitable for the pharmaceutical compositions disclosed herein include buffers (e.g., neutral buffered saline, phosphate buffered saline); carbohydrates (e.g., glucose, mannose, sucrose, dextran, mannitol); polypeptides or amino acids (e.g., glycine); antioxidants; chelating agents (e.g., EDTA, glutathione); adjuvants (e.g., aluminum hydroxide); surfactants (Polysorbate 80, Polysorbate 20, or Pluronic F68); glycerol; sorbitol; mannitol; polyethyleneglycol; and preservatives. In some embodiments, the vector is formulated for delivery through injection by a needle carrying syringe. In some embodiments, the composition is formulated for delivery by electroporation. In some embodiments, the composition is formulated for delivery by chemical method. In some embodiments, the pharmaceutical compositions comprise a virus vector or a non-viral vector.

In some embodiments, pharmaceutical compositions are in the form of a solution (e.g., a liquid). The solution may be formulated for injection, e.g., intravenous or subcutaneous injection. In some embodiments, the pH of the solution is about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, or about 9. In some embodiments, the pH is 7 to 7.5, 7.5 to 8, 8 to 8.5, 8.5 to 9, or 7 to 8.5. In some cases, the pH of the solution is less than 7. In some cases, the pH is greater than 7.

### IX. Systems for Detection

Disclosed herein, in some aspects, are systems for detecting a target nucleic acid, comprising any one of the effector proteins described herein. In some embodiments, systems comprise a guide nucleic acid. Systems may be used to detect a target nucleic acid. In some embodiments, systems comprise an effector protein described herein, a reagent, support medium, or a combination thereof. In some embodiments, systems comprise a fusion protein described herein. In some embodiments, effector proteins comprise an amino acid sequence that is at least 70%, at least 75%, at least 80%, at least 85%, at least 90%, at least 95%, or 100% identical to any one of the amino acid sequences selected from SEQ ID NOS: 1-78 and 499. In some embodiments, the amino acid sequence of the effector protein is at least 70%, at least 75%, at least 80%, at least 85%, at least 90%, at least 95%, or 100% identical to any one of the amino acid sequences selected from SEQ ID NOS: 1-78 and 499.

Systems may be used for detecting the presence of a target nucleic acid associated with or causative of a disease, such as cancer, a genetic disorder, or an infection. In some embodiments, systems are useful for phenotyping, genotyping, or determining ancestry. Unless specified otherwise, systems include kits and may be referred to as kits. Unless specified otherwise, systems include devices and may also be referred to as devices. Systems described herein may be provided in the form of a companion diagnostic assay or device, a point-of-care assay or device, or an over-the-counter diagnostic assay/device.

In some embodiments, in vitro can be used to describe an event that takes places in a container for holding laboratory reagents such that it is separated from the biological source from which the material is obtained. In vitro assays can encompass cell-based assays in which living or dead cells are employed. In vitro assays can also encompass a cell-free assay in which no intact cells are employed. In some cases, in vivo can be used to describe an event that takes place in a subject's body. In some cases, ex vivo can be used to describe an event that takes place outside of a subject's body. An ex vivo assay is not performed on a subject. Rather, it is performed upon a sample separate from a subject. An example of an ex vivo assay performed on a sample is an in vitro assay.

Reagents and effector proteins of various systems may be provided in a reagent chamber or on a support medium. Alternatively, the reagent and/or effector protein may be contacted with the reagent chamber or the support medium by the individual using the system. An exemplary reagent chamber is a test well or container. The opening of the reagent chamber may be large enough to accommodate the support medium. Optionally, the system comprises a buffer and a dropper. The buffer may be provided in a dropper bottle for ease of dispensing. The dropper may be disposable and transfer a fixed volume. The dropper may be used to place a sample into the reagent chamber or on the support medium.

### System Solutions

In general, systems comprise a solution in which the activity of an effector protein occurs. Often, the solution comprises or consists essentially of a buffer. The solution or buffer may comprise a buffering agent, a salt, a crowding agent, a detergent, a reducing agent, a competitor, or a combination thereof. Often the buffer is the primary component or the basis for the solution in which the activity occurs. Thus, concentrations for components of buffers described herein (e.g., buffering agents, salts, crowding agents, detergents, reducing agents, and competitors) are the same or essentially the same as the concentration of these components in the solution in which the activity occurs. In some embodiments, a buffer is required for cell lysis activity or viral lysis activity.

In some embodiments, systems comprise a buffer, wherein the buffer comprise at least one buffering agent. Exemplary buffering agents include HEPES, TRIS, MES, ADA, PIPES, ACES, MOPSO, BIS-TRIS propane, BES, MOPS, TES, DISO, Trizma, TRICINE, GLY-GLY, HEPPS, BICINE, TAPS, A MPD, A MPSO, CHES, CAPSO, AMP, CAPS, phosphate, citrate, acetate, imidazole, or any combination thereof. In some embodiments, the concentration of the buffering agent in the buffer is 1 mM to 200 mM. A buffer compatible with an effector protein may comprise a buffering agent at a concentration of 10 mM to 30 mM. A buffer compatible with an effector protein may comprise a buffering agent at a concentration of about 20 mM. A buffering agent may provide a pH for the buffer or the solution in which the activity of the effector protein occurs. The pH may be 3 to 4, 3.5 to 4.5, 4 to 5, 4.5 to 5.5, 5 to 6, 5.5 to 6.5, 6 to 7, 6.5 to 7.5, 7 to 8, 7.5 to 8.5, 8 to 9, 8.5 to 9.5, 9 to 10, or 9.5 to 10.5.

In some embodiments, systems comprise a solution, wherein the solution comprises at least one salt. In some embodiments, the at least one salt is selected from potassium acetate, magnesium acetate, sodium chloride, potassium chloride, magnesium chloride, calcium chloride, and any combination thereof. In some embodiments, the concentration of the at least one salt in the solution is 5 mM to 100 mM, 5 mM to 10 mM, 1 mM to 60 mM, or 1 mM to 10 mM. In some embodiments, the concentration of the at least one salt is about 105 mM. In some embodiments, the concentration of the at least one salt is about 55 mM. In some embodiments, the concentration of the at least one salt is about 7 mM. In some embodiments, the solution comprises potassium acetate and magnesium acetate. In some embodiments, the solution comprises sodium chloride and magnesium chloride. In some embodiments, the solution comprises potassium chloride and magnesium chloride. In some embodiments, the salt is a magnesium salt and the concentration of magnesium in the solution is at least 5 mM, 7 mM, at least 9 mM, at least 11 mM, at least 13 mM, or at least 15 mM. In some embodiments, the concentration of magnesium is less than 20 mM, less than 18 mM, or less than 16 mM.

In some embodiments, systems comprise a solution, wherein the solution comprises at least one crowding agent. A crowding agent may reduce the volume of solvent available for other molecules in the solution, thereby increasing the effective concentrations of said molecules. Exemplary crowding agents include glycerol and bovine serum albumin. In some embodiments, the crowding agent is glycerol. In some embodiments, the concentration of the crowding agent in the solution is 0.01% (v/v) to 10% (v/v). In some embodiments, the concentration of the crowding agent in the solution is 0.5% (v/v) to 10% (v/v).

In some embodiments, systems comprise a solution, wherein the solution comprises at least one detergent. Exemplary detergents include Tween, Triton-X, and IGEPAL. A solution may comprise Tween, Triton-X, or any combination thereof. A solution may comprise Triton-X. A solution may comprise IGEPAL CA-630. In some embodiments, the concentration of the detergent in the solution is 2% (v/v) or less. In some embodiments, the concentration of the detergent in the solution is 1% (v/v) or less. In some embodiments, the concentration of the detergent in the solution is 0.00001% (v/v) to 0.01% (v/v). In some embodiments, the concentration of the detergent in the solution is about 0.01% (v/v).

In some embodiments, systems comprise a solution, wherein the solution comprises at least one reducing agent. Exemplary reducing agents comprise dithiothreitol (DTT), β-mercaptoethanol (BME), or tris(2-carboxyethyl) phosphine (TCEP). In some embodiments, the reducing agent is DTT. In some embodiments, the concentration of the reducing agent in the solution is 0.01 mM to 100 mM. In some embodiments, the concentration of the reducing agent in the solution is 0.1 mM to 10 mM. In some embodiments, the concentration of the reducing agent in the solution is 0.5 mM to 2 mM. In some embodiments, the concentration of the reducing agent in the solution is 0.01 mM to 100 mM. In some embodiments, the concentration of the reducing agent in the solution is 0.1 mM to 10 mM. In some embodiments, the concentration of the reducing agent in the solution is about 1 mM.

In some embodiments, systems comprise a solution, wherein the solution comprises a competitor. In general, competitors compete with the target nucleic acid or the reporter nucleic acid for cleavage by the effector protein or a dimer thereof. Exemplary competitors include heparin, and imidazole, and salmon sperm DNA. In some embodiments, the concentration of the competitor in the solution is 1 μg/mL to 100 μg/mL. In some embodiments, the concentration of the competitor in the solution is 40 μg/mL to 60 μg/mL.

In some embodiments, systems comprise a solution, wherein the solution comprises a co-factor. In some embodiments, the co-factor allows an effector protein or a multimeric complex thereof to perform a function, including pre-crRNA processing and/or target nucleic acid cleavage. The suitability of a cofactor for an effector protein or a multimeric complex thereof may be assessed, such as by methods based on those described by Sundaresan et al. (Cell Rep. 2017 Dec. 26; 21(13): 3728-3739). In some embodiments, an effector or a multimeric complex thereof forms a complex with a co-factor. In some embodiments, the co-factor is a divalent metal ion. In some embodiments, the divalent metal ion is selected from Mg²⁺, Mn²⁺, Zn²⁺, Ca²⁺, Cu²⁺. In some embodiments, the divalent metal ion is Mg²⁺. In some embodiments, the co-factor is Mg²⁺.

### Reporters

In some embodiments, systems disclosed herein comprise a reporter. By way of non-limiting and illustrative example, a reporter may comprise a single stranded nucleic acid and a detection moiety (e.g., a labeled single stranded RNA reporter), wherein the nucleic acid is capable of being cleaved by an effector protein (e.g., a CRISPR/Cas protein as disclosed herein) or a multimeric complex thereof, releasing the detection moiety, and generating a detectable signal. In some embodiments, a detectable signal comprises a signal that can be detected using optical, fluorescent, chemiluminescent, electrochemical and other detection methods known in the art. The effector proteins disclosed herein, activated upon hybridization of a guide nucleic acid to a target nucleic acid, may cleave the reporter. Cleaving the “reporter” may be referred to herein as cleaving the “reporter nucleic acid,” the “reporter molecule,” or the “nucleic acid of the reporter.” Reporters may comprise RNA. Reporters may comprise DNA. Reporters may be double-stranded. Reporters may be single-stranded.

In some embodiments, reporters comprise a protein capable of generating a signal. A signal may be a calorimetric, potentiometric, amperometric, optical (e.g., fluorescent, colorimetric, etc.), or piezo-electric signal. In some embodiments, the reporter comprises a detection moiety. Suitable detectable labels and/or moieties that may provide a signal include, but are not limited to, an enzyme, a radioisotope, a member of a specific binding pair; a fluorophore; a fluorescent protein; a quantum dot; and the like.

In some embodiments, the reporter comprises a detection moiety and a quenching moiety. In some embodiments, the reporter comprises a cleavage site, wherein the detection moiety is located at a first site on the reporter and the quenching moiety is located at a second site on the reporter, wherein the first site and the second site are separated by the cleavage site. Sometimes the quenching moiety is a fluorescence quenching moiety. In some embodiments, the quenching moiety is 5′ to the cleavage site and the detection moiety is 3′ to the cleavage site. In some embodiments, the detection moiety is 5′ to the cleavage site and the quenching moiety is 3′ to the cleavage site. Sometimes the quenching moiety is at the 5′ terminus of the nucleic acid of a reporter. Sometimes the detection moiety is at the 3′ terminus of the nucleic acid of a reporter. In some embodiments, the detection moiety is at the 5′ terminus of the nucleic acid of a reporter. In some embodiments, the quenching moiety is at the 3′ terminus of the nucleic acid of a reporter.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilised EGFP (dEGFP), destabilised ECFP (dECFP), destabilised EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2(12), mRFP1, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Suitable enzymes include, but are not limited to, horseradish peroxidase (HRP), alkaline phosphatase (AP), beta-galactosidase (GAL), glucose-6-phosphate dehydrogenase, beta-N-acetylglucosaminidase, β-glucuronidase, invertase, Xanthine Oxidase, firefly luciferase, and glucose oxidase (GO).

In some embodiments, the detection moiety comprises an invertase. The substrate of the invertase may be sucrose. A DNS reagent may be included in the system to produce a colorimetric change when the invertase converts sucrose to glucose. In some embodiments, the reporter nucleic acid and invertase are conjugated using a heterobifunctional linker via sulfo-SMCC chemistry.

Suitable fluorophores may provide a detectable fluorescence signal in the same range as 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). Non-limiting examples of fluorophores are fluorescein amidite, 6-Fluorescein, IRDye 700, TYE 665, Alex Fluor 594, or ATTO TM 633 (NHS Ester). The fluorophore may be an infrared fluorophore. The fluorophore may emit fluorescence in the range of 500 nm and 720 nm. In some embodiments, the fluorophore emits fluorescence at a wavelength of 700 nm or higher. In other cases, the fluorophore emits fluorescence at about 665 nm. In some embodiments, the fluorophore emits fluorescence in the range of 500 nm to 520 nm, 500 nm to 540 nm, 500 nm to 590 nm, 590 nm to 600 nm, 600 nm to 610 nm, 610 nm to 620 nm, 620 nm to 630 nm, 630 nm to 640 nm, 640 nm to 650 nm, 650 nm to 660 nm, 660 nm to 670 nm, 670 nm to 680 nm, 690 nm to 690 nm, 690 nm to 700 nm, 700 nm to 710 nm, 710 nm to 720 nm, or 720 nm to 730 nm. In some embodiments, the fluorophore emits fluorescence in the range 450 nm to 750 nm, 500 nm to 650 nm, or 550 to 650 nm.

Systems may comprise a quenching moiety. A quenching moiety may be chosen based on its ability to quench the detection moiety. A quenching moiety may be a non-fluorescent fluorescence quencher. A quenching moiety may quench a detection moiety that emits fluorescence in the range of 500 nm and 720 nm. A quenching moiety may quench a detection moiety that emits fluorescence in the range of 500 nm and 720 nm. In some embodiments, the quenching moiety quenches a detection moiety that emits fluorescence at a wavelength of 700 nm or higher. In other cases, the quenching moiety quenches a detection moiety that emits fluorescence at about 660 nm or about 670 nm. In some embodiments, the quenching moiety quenches a detection moiety that emits fluorescence in the range of 500 to 520, 500 to 540, 500 to 590, 590 to 600, 600 to 610, 610 to 620, 620 to 630, 630 to 640, 640 to 650, 650 to 660, 660 to 670, 670 to 680, 690 to 690, 690 to 700, 700 to 710, 710 to 720, or 720 to 730 nm. In some embodiments, the quenching moiety quenches a detection moiety that emits fluorescence in the range 450 nm to 750 nm, 500 nm to 650 nm, or 550 to 650 nm. A quenching moiety may quench fluorescein amidite, 6-Fluorescein, IRDye 700, TYE 665, Alex Fluor 594, or ATTO TM 633 (NHS Ester). A quenching moiety may be Iowa Black RQ, Iowa Black FQ or IRDye QC-1 Quencher. A quenching moiety may quench fluorescein amidite, 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). A quenching moiety may be Iowa Black RQ (Integrated DNA Technologies), Iowa Black FQ (Integrated DNA Technologies) or IRDye QC-1 Quencher (LiCor). Any of the quenching moieties described herein may be from any commercially available source, may be an alternative with a similar function, a generic, or a non-tradename of the quenching moieties listed.

The generation of the detectable signal from the release of the detection moiety may indicate that cleavage by the effector protein has occurred and that the sample contains the target nucleic acid. In some embodiments, the detection moiety comprises a fluorescent dye. Sometimes the detection moiety comprises a fluorescence resonance energy transfer (FRET) pair. In some embodiments, the detection moiety comprises an infrared (IR) dye. In some embodiments, the detection moiety comprises an ultraviolet (UV) dye. Alternatively, or in combination, the detection moiety comprises a protein. Sometimes the detection moiety comprises a biotin. Sometimes the detection moiety comprises at least one of avidin or streptavidin. In some embodiments, the detection moiety comprises a polysaccharide, a polymer, or a nanoparticle. In some embodiments, the detection moiety comprises a gold nanoparticle or a latex nanoparticle.

A detection moiety may be any moiety capable of generating a detectable product or detectable signal upon cleavage of the reporter by the effector protein. The detectable product may be a detectable unit generated from the detectable moiety and capable of emitting a detectable signal as described herein. In some embodiments, the detectable product further comprises a detectable label, a fluorophore, a reporter, or a combination thereof. In some embodiments, the detectable product comprises RNA, DNA, or both. In some embodiments, the detectable product is configured to generate a signal indicative of the presence or absence of the target nucleic acid in, for instance, a cell or a sample.

A detection moiety may be any moiety capable of generating a calorimetric, potentiometric, amperometric, optical (e.g., fluorescent, colorimetric, etc.), or piezo-electric signal. A nucleic acid of a reporter, sometimes, is protein-nucleic acid that is capable of generating a calorimetric, potentiometric, amperometric, optical (e.g., fluorescent, colorimetric, etc.), or piezo-electric signal upon cleavage of the nucleic acid. Often a calorimetric signal is heat produced after cleavage of the nucleic acids of a reporter. Sometimes, a calorimetric signal is heat absorbed after cleavage of the nucleic acids of a reporter. A potentiometric signal, for example, is electrical potential produced after cleavage of the nucleic acids of a reporter. An amperometric signal may be movement of electrons produced after the cleavage of nucleic acid of a reporter. Often, the signal is an optical signal, such as a colorimetric signal or a fluorescence signal. An optical signal is, for example, a light output produced after the cleavage of the nucleic acids of a reporter. Sometimes, an optical signal is a change in light absorbance between before and after the cleavage of nucleic acids of a reporter. Often, a piezo-electric signal is a change in mass between before and after the cleavage of the nucleic acid of a reporter.

The detectable signal may be a colorimetric signal or a signal visible by eye. In some embodiments, the detectable signal may be fluorescent, electrical, chemical, electrochemical, or magnetic. In some embodiments, the first detection signal may be generated by binding of the detection moiety to the capture molecule in the detection region, where the first detection signal indicates that the sample contained the target nucleic acid. Sometimes systems are capable of detecting more than one type of target nucleic acid, wherein the system comprises more than one type of guide nucleic acid and more than one type of reporter nucleic acid. In some embodiments, the detectable signal may be generated directly by the cleavage event. Alternatively, or in combination, the detectable signal may be generated indirectly by the signal event. Sometimes the detectable signal is not a fluorescent signal. In some embodiments, the detectable signal may be a colorimetric or color-based signal. In some embodiments, the detected target nucleic acid may be identified based on its spatial location on the detection region of the support medium. In some embodiments, the second detectable signal may be generated in a spatially distinct location than the first generated signal.

In some embodiments, the reporter nucleic acid is a single-stranded nucleic acid sequence comprising ribonucleotides. The nucleic acid of a reporter may be a single-stranded nucleic acid sequence comprising at least one ribonucleotide. In some embodiments, the nucleic acid of a reporter is a single-stranded nucleic acid comprising at least one ribonucleotide residue at an internal position that functions as a cleavage site. In some embodiments, the nucleic acid of a reporter comprises at least 2, at least 3, at least 4, at least 5, at least 6, at least 7, at least 8, at least 9, or at least 10 ribonucleotide residues at an internal position. In some embodiments, the nucleic acid of a reporter comprises from 2 to 10, from 3 to 9, from 4 to 8, or from 5 to 7 ribonucleotide residues at an internal position. Sometimes the ribonucleotide residues are continuous. Alternatively, the ribonucleotide residues are interspersed in between non-ribonucleotide residues. In some embodiments, the nucleic acid of a reporter has only ribonucleotide residues. In some embodiments, the nucleic acid of a reporter has only deoxyribonucleotide residues. In some embodiments, the nucleic acid comprises nucleotides resistant to cleavage by the effector protein described herein. In some embodiments, the nucleic acid of a reporter comprises synthetic nucleotides. In some embodiments, the nucleic acid of a reporter comprises at least one ribonucleotide residue and at least one non-ribonucleotide residue.

In some embodiments, the nucleic acid of a reporter comprises at least one uracil ribonucleotide. In some embodiments, the nucleic acid of a reporter comprises at least two uracil ribonucleotides. Sometimes the nucleic acid of a reporter has only uracil ribonucleotides. In some embodiments, the nucleic acid of a reporter comprises at least one adenine ribonucleotide. In some embodiments, the nucleic acid of a reporter comprises at least two adenine ribonucleotides. In some embodiments, the nucleic acid of a reporter has only adenine ribonucleotides. In some embodiments, the nucleic acid of a reporter comprises at least one cytosine ribonucleotide. In some embodiments, the nucleic acid of a reporter comprises at least two cytosine ribonucleotides. In some embodiments, the nucleic acid of a reporter comprises at least one guanine ribonucleotide. In some embodiments, the nucleic acid of a reporter comprises at least two guanine ribonucleotides. In some embodiments, a nucleic acid of a reporter comprises a single unmodified ribonucleotide. In some embodiments, a nucleic acid of a reporter comprises only unmodified deoxyribonucleotides.

In some embodiments, the nucleic acid of a reporter is 5 to 20, 5 to 15, 5 to 10, 7 to 20, 7 to 15, or 7 to 10 nucleotides in length. In some embodiments, the nucleic acid of a reporter is 3 to 20, 4 to 10, 5 to 10, or 5 to 8 nucleotides in length. In some embodiments, the nucleic acid of a reporter is 5 to 12 nucleotides in length. In some embodiments, the reporter nucleic acid is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 nucleotides in length. In some embodiments, the reporter nucleic acid is 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length.

In some embodiments, systems comprise a plurality of reporters. The plurality of reporters may comprise a plurality of signals. In some embodiments, systems comprise at least 2, at least 3, at least 4, at least 5, at least 6, at least 7, at least 8, at least 9, at least 10, at least 11, at least 12, at least 13, at least 14, at least 15, at least 20, at least 30, at least 40, or at least 50 reporters. In some embodiments, there are 2 to 50, 3 to 40, 4 to 30, 5 to 20, or 6 to 10 different reporters.

In some embodiments, systems comprise an effector protein and a reporter nucleic acid configured to undergo trans cleavage by the effector protein. Trans cleavage of the reporter may generate a signal from the reporter or alter a signal from the reporter. In some embodiments, the signal is an optical signal, such as a fluorescence signal or absorbance band. Trans cleavage of the reporter may alter the wavelength, intensity, or polarization of the optical signal. For example, the reporter may comprise a fluorophore and a quencher, such that trans cleavage of the reporter separates the fluorophore and the quencher thereby increasing a fluorescence signal from the fluorophore. Herein, detection of reporter cleavage to determine the presence of a target nucleic acid sequence may be referred to as ‘DETECTR’. In some embodiments described herein is a method of assaying for a target nucleic acid in a sample comprising contacting the target nucleic acid with an effector protein, a non-naturally occurring guide nucleic acid that hybridizes to a segment of the target nucleic acid, and a reporter nucleic acid, and assaying for a change in a signal, wherein the change in the signal is produced by cleavage of the reporter nucleic acid.

In the presence of a large amount of non-target nucleic acids, an activity of an effector protein (e.g., an effector protein as disclosed herein) may be inhibited. This is because the activated effector proteins collaterally cleave any nucleic acids. If total nucleic acids are present in large amounts, they may outcompete reporters for the effector proteins. In some embodiments, systems comprise an excess of reporter(s), such that when the system is operated and a solution of the system comprising the reporter is combined with a sample comprising a target nucleic acid, the concentration of the reporter in the combined solution-sample is greater than the concentration of the target nucleic acid. In some embodiments, the sample comprises amplified target nucleic acid. In some embodiments, the sample comprises an unamplified target nucleic acid. In some embodiments, the concentration of the reporter is greater than the concentration of target nucleic acids and non-target nucleic acids. The non-target nucleic acids may be from the original sample, either lysed or unlysed. The non-target nucleic acids may comprise byproducts of amplification. In some embodiments, systems comprise a reporter wherein the concentration of the reporter in a solution 1.5 fold, at least 2 fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 16 fold, at least 17 fold, at least 18 fold, at least 19 fold, at least 20 fold, at least 30 fold, at least 40 fold, at least 50 fold, at least 60 fold, at least 70 fold, at least 80 fold, at least 90 fold, at least 100 fold excess of total nucleic acids. In some embodiments, systems comprise a reporter wherein the concentration of the reporter in a solution 1.5 fold to 100 fold, 2 fold to 10 fold, 10 fold to 20 fold, 20 fold to 30 fold, 30 fold to 40 fold, 40 fold to 50 fold, 50 fold to 60 fold, 60 fold to 70 fold, 70 fold to 80 fold, 80 fold to 90 fold, 90 fold to 100 fold, 1.5 fold to 10 fold, 1.5 fold to 20 fold, 10 fold to 40 fold, 20 fold to 60 fold, or 10 fold to 80 fold excess of total nucleic acids.

In some embodiments, systems may comprise one or more hydrogels. In some embodiments, each of the one or more hydrogels comprise one or more reporter molecules as described herein. In some embodiments, the reporter and/or the detection moiety can be immobilized on a support medium, such as a surface or hydrogel. In some embodiments, the detection moiety binds to a capture molecule on the support medium or hydrogel to be immobilized. The detectable signal can be visualized on the support medium or hydrogel to assess the presence or concentration of one or more target nucleic acids associated with an ailment, such as a disease, cancer, or genetic disorder.

### Amplification Reagents/Components

In some embodiments, systems described herein comprise a reagent or component for amplifying a nucleic acid. Non-limiting examples of reagents for amplifying a nucleic acid include polymerases, primers, and nucleotides. In some embodiments, systems comprise reagents for nucleic acid amplification of a target nucleic acid in a sample. Nucleic acid amplification of the target nucleic acid may improve at least one of sensitivity, specificity, or accuracy of the assay in detecting the target nucleic acid. In some embodiments, nucleic acid amplification is isothermal nucleic acid amplification, providing for the use of the system or system in remote regions or low resource settings without specialized equipment for amplification. In some embodiments, amplification of the target nucleic acid increases the concentration of the target nucleic acid in the sample relative to the concentration of nucleic acids that do not correspond to the target nucleic acid.

The reagents for nucleic acid amplification may comprise a recombinase, an oligonucleotide primer, a single-stranded DNA binding (SSB) protein, a polymerase, or a combination thereof that is suitable for an amplification reaction. Non-limiting examples of amplification reactions are transcription mediated amplification (TMA), helicase dependent amplification (HDA), or circular helicase dependent amplification (cHDA), strand displacement amplification (SDA), recombinase polymerase amplification (RPA), loop mediated amplification (LAMP), exponential amplification reaction (EXPAR), rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), and improved multiple displacement amplification (IMDA).

In some embodiments, systems comprise a PCR tube, a PCR well or a PCR plate. The wells of the PCR plate may be pre-aliquoted with the reagent for amplifying a nucleic acid, as well as a guide nucleic acid, an effector protein, a multimeric complex, or any combination thereof. The wells of the PCR plate may be pre-aliquoted with a guide nucleic acid targeting a target sequence, an effector protein capable of being activated when complexed with the guide nucleic acid and the target sequence, and at least one population of a single stranded reporter nucleic acid comprising a detection moiety. A user may thus add the biological sample of interest to a well of the pre-aliquoted PCR plate and measure for the detectable signal with a fluorescent light reader or a visible light reader.

In some embodiments, systems comprise a PCR plate; a guide nucleic acid targeting a target sequence; an effector protein capable of being activated when complexed with the guide nucleic acid and the target sequence; and a single stranded reporter nucleic acid comprising a detection moiety, wherein the reporter nucleic acid is capable of being cleaved by the activated nuclease, thereby generating a detectable signal.

In some embodiments, systems comprise a support medium; a guide nucleic acid targeting a target sequence; and an effector protein capable of being activated when complexed with the guide nucleic acid and the target sequence. In some embodiments, nucleic acid amplification is performed in a nucleic acid amplification region on the support medium. Alternatively, or in combination, the nucleic acid amplification is performed in a reagent chamber, and the resulting sample is applied to the support medium.

In some embodiments, a system for modifying a target nucleic acid comprises a PCR plate; a guide nucleic acid targeting a target sequence; and an effector protein capable of being activated when complexed with the guide nucleic acid and the target sequence. The wells of the PCR plate may be pre-aliquoted with the guide nucleic acid targeting a target sequence, and an effector protein capable of being activated when complexed with the guide nucleic acid and the target sequence. A user may thus add the biological sample of interest to a well of the pre-aliquoted PCR plate.

In some embodiments, wells of the PCR plate may be pre-aliquoted with a guide nucleic acid targeting a target sequence, an effector protein capable of being activated when complexed with the guide nucleic acid and the target sequence, and at least one population of a single stranded reporter nucleic acid comprising a detection moiety. In some embodiments, the reporter nucleic acid is capable of being cleaved by the activated nuclease, thereby generating a detectable signal. A user may thus add the biological sample of interest to a well of the pre-aliquoted PCR plate and measure for the detectable signal with a fluorescent light reader or a visible light reader.

Often, the nucleic acid amplification is performed for no greater than 1, 2, 3, 4, 5, 6, 7, 8, 9, 10, 11, 12, 13, 14, 15, 16, 17, 18, 19, 20, 25, 30, 40, 50, or 60 minutes, or any value 1 to 60 minutes. Sometimes, the nucleic acid amplification is performed for 1 to 60, 5 to 55, 10 to 50, 15 to 45, 20 to 40, or 25 to 35 minutes. Sometimes, the nucleic acid amplification reaction is performed at a temperature of around 20-45° C. In some embodiments, the nucleic acid amplification reaction is performed at a temperature no greater than 20° C., 25° C., 30° C., 35° C., 37° C., 40° C., 45° C., or any value 20° C. to 45° C. In some embodiments, the nucleic acid amplification reaction is performed at a temperature of at least 20° C., 25° C., 30° C., 35° C., 37° C., 40° C., or 45° C., or any value 20° C. to 45° C. In some embodiments, the nucleic acid amplification reaction is performed at a temperature of 20° C. to 45° C., 25° C. to 40° C., 30° C. to 40° C., or 35° C. to 40° C.

Often, systems comprise primers for amplifying a target nucleic acid to produce an amplification product comprising the target nucleic acid and a PAM. For instance, at least one of the primers may comprise the PAM that is incorporated into the amplification product during amplification. The compositions for amplification of target nucleic acids and methods of use thereof, as described herein, are compatible with any of the methods disclosed herein including methods of assaying for at least one base difference (e.g., assaying for a SNP or a base mutation) in a target nucleic acid sequence, methods of assaying for a target nucleic acid that lacks a PAM by amplifying the target nucleic acid sequence to introduce a PAM, and compositions used in introducing a PAM via amplification into the target nucleic acid sequence.

### Additional System Components

In some embodiments, systems include a package, carrier, or container that is compartmentalized to receive one or more containers such as vials, tubes, and the like, each of the container(s) comprising one of the separate elements to be used in a method described herein. Suitable containers include, for example, test wells, bottles, vials, and test tubes. In one embodiment, the containers are formed from a variety of materials such as glass, plastic, or polymers. The system or systems described herein contain packaging materials. Examples of packaging materials include, but are not limited to, pouches, blister packs, bottles, tubes, bags, containers, bottles, and any packaging material suitable for intended mode of use.

A system may include labels listing contents and/or instructions for use, or package inserts with instructions for use. A set of instructions will also typically be included. In one embodiment, a label is on or associated with the container. In some embodiments, a label is on a container when letters, numbers or other characters forming the label are attached, molded, or etched into the container itself; a label is associated with a container when it is present within a receptacle or cater that also holds the container, e.g., as a package insert. In one embodiment, a label is used to indicate that the contents are to be used for a specific therapeutic application. The label also indicates directions for use of the contents, such as in the methods described herein. After packaging the formed product and wrapping or boxing to maintain a sterile barrier, the product may be terminally sterilized by heat sterilization, gas sterilization, gamma irradiation, or by electron beam sterilization. Alternatively, the product may be prepared and packaged by aseptic processing.

In some embodiments, systems comprise a solid support. An RNP or effector protein may be attached to a solid support. The solid support may be an electrode or a bead. The bead may be a magnetic bead. Upon cleavage, the RNP is liberated from the solid support and interacts with other mixtures. For example, upon cleavage of the nucleic acid of the RNP, the effector protein of the RNP flows through a chamber into a mixture comprising a substrate. When the effector protein meets the substrate, a reaction occurs, such as a colorimetric reaction, which is then detected. As another example, the protein is an enzyme substrate, and upon cleavage of the nucleic acid of the enzyme substrate-nucleic acid, the enzyme flows through a chamber into a mixture comprising the enzyme. When the enzyme substrate meets the enzyme, a reaction occurs, such as a calorimetric reaction, which is then detected.

### Certain System Conditions

In some embodiments, systems and methods are employed under certain conditions that enhance an activity of the effector protein relative to alternative conditions, as measured by a detectable signal released from cleavage of a reporter in the presence of the target nucleic acid. The detectable signal may be generated at about the rate of trans cleavage of a reporter nucleic acid. In some embodiments, the reporter nucleic acid is a homopolymeric reporter nucleic acid comprising 5 to 20 consecutive adenines, 5 to 20 consecutive thymines, 5 to 20 consecutive cytosines, or 5 to 20 consecutive guanines. In some embodiments, the reporter is an RNA-FQ reporter.

In some embodiments, effector proteins disclosed herein recognize, bind, or are activated by, different target nucleic acids having different sequences, but are active toward the same reporter nucleic acid, allowing for facile multiplexing in a single assay having a single ssRNA-FQ reporter.

In some embodiments, systems are employed under certain conditions that enhance trans cleavage activity of an effector protein. In some embodiments, under certain conditions, transcolatteral cleavage occurs at a rate of at least 0.005 mmol/min, at least 0.01 mmol/min, at least 0.05 mmol/min, at least 0.1 mmol/min, at least 0.2 mmol/min, at least 0.5 mmol/min, or at least 1 mmol/min. In some embodiments, systems and methods are employed under certain conditions that enhance cis-cleavage activity of the effector protein.

Certain conditions that may enhance the activity of an effector protein include a certain salt presence or salt concentration of the solution in which the activity occurs. For example, cis-cleavage activity of an effector protein may be inhibited or halted by a high salt concentration. The salt may be a sodium salt, a potassium salt, or a magnesium salt. In some embodiments, the salt is NaCl. In some embodiments, the salt is KNO₃. In some embodiments, the salt concentration is less than 150 mM, less than 125 mM, less than 100 mM, less than 75 mM, less than 50 mM, or less than 25 mM.

Certain conditions that may enhance the activity of an effector protein include the pH of a solution in which the activity. For example, increasing pH may enhance trans cleavage activity. For example, the rate of trans cleavage activity may increase with increase in pH up to pH 9. In some embodiments, the pH is about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, or about 9. In some embodiments, the pH is 7 to 7.5, 7.5 to 8, 8 to 8.5, 8.5 to 9, or 7 to 8.5. In some embodiments, the pH is less than 7. In some embodiments, the pH is greater than 7.

Certain conditions that may enhance the activity of an effector protein includes the temperature at which the activity is performed. In some embodiments, the temperature is about 25° C. to about 50° C. In some embodiments, the temperature is about 20° C. to about 40° C., about 30° C. to about 50° C., or about 40° C. to about 60° C. In some embodiments, the temperature is about 25° C., about 30° C., about 35° C., about 40° C., about 45° C., or about 50° C.

### X. Methods of Detecting a Target Nucleic Acid

Provided herein are methods of detecting target nucleic acids. Methods may comprise detecting target nucleic acids with compositions or systems described herein. In some embodiments, the methods of detecting a target nucleic acid comprising: a) contacting the target nucleic acid with a composition comprising an effector protein as described herein, a guide nucleic acid as described herein, and a reporter nucleic acid that is cleaved in the presence of the effector protein, the guide nucleic acid, and the target nucleic acid; and b) detecting a signal produced by cleavage of the reporter nucleic acid, thereby detecting the target nucleic acid in the sample. In some embodiments, the methods result in trans cleavage of the reporter nucleic acid. In some embodiments, the methods result in cis cleavage of the reporter nucleic acid. In some embodiments, the reporter nucleic acid is a single stranded nucleic acid. In some embodiments, the reporter comprises a detection moiety. In some embodiments, the reporter nucleic acid is capable of being cleaved by the effector protein. In some embodiments, a cleaved reporter nucleic acid generates a detectable product or a first detectable signal. In some embodiments, the first detectable signal is a change in color. In some embodiments, the change is color is measured indicating presence of the target nucleic acid. In some embodiments, the first detectable signal is measured on a support medium.

Methods may comprise detecting a target nucleic acid in a sample, e.g., a cell lysate, a biological fluid, or environmental sample. Methods may comprise detecting a target nucleic acid in a cell. In some embodiments, methods of detecting a target nucleic acid in a sample or cell comprises contacting the sample or cell with an effector protein or a multimeric complex thereof, a guide nucleic acid, wherein at least a portion of the guide nucleic acid is complementary to at least a portion of the target nucleic acid, and a reporter nucleic acid that is cleaved in the presence of the effector protein, the guide nucleic acid, and the target nucleic acid, and detecting a signal produced by cleavage of the reporter nucleic acid, thereby detecting the target nucleic acid in the sample. In some embodiments, methods result in trans cleavage of the reporter nucleic acid. In some embodiments, methods result in cis cleavage of the reporter nucleic acid.

In some embodiments, methods of detecting comprise contacting a target nucleic acid, a cell comprising the target nucleic acid, or a sample comprising a target nucleic acid with an effector protein that comprises an amino acid sequence that is at least is at least 70%, at least 75%, at least 80%, at least 85%, at least 90%, at least 92%, at least 95%, at least 97%, at least 98%, at least 99%, or 100% identical to any one of SEQ ID NOS: 1-78 and 499. In some embodiments, the amino acid sequence of the effector protein is at least 70%, at least 75%, at least 80%, at least 85%, at least 90%, at least 92%, at least 95%, at least 97%, at least 98%, at least 99%, or 100% identical to any one of SEQ ID NOS: 1-78 and 499. In some embodiments, a reporter and/or a reporter nucleic acid comprise a non-target nucleic acid molecule that can provide a detectable signal upon cleavage by an effector protein. Examples of detectable signals and detectable moieties that generate detectable signals are provided herein.

In some embodiments, target nucleic acid comprises a nucleic acid that is selected as the nucleic acid for modification, binding, hybridization or any other activity of or interaction with a nucleic acid, protein, polypeptide, or peptide described herein. A target nucleic acid may comprise RNA, DNA, or a combination thereof. A target nucleic acid may be single-stranded (e.g., single-stranded RNA or single-stranded DNA) or double-stranded (e.g., double-stranded DNA). The target nucleic acid may be from any organism, including, but not limited to, a bacterium, a virus, a parasite, a protozoon, a fungus, a mammal, a plant, and an insect. As another non-limiting example, the target nucleic acid may be responsible for a disease, contain a mutation (e.g., single strand polymorphism, point mutation, insertion, or deletion), be contained in an amplicon, or be uniquely identifiable from the surrounding nucleic acids (e.g., contain a unique sequence of nucleotides).

Methods may comprise contacting the sample to a complex comprising a guide nucleic acid comprising a segment that is reverse complementary to a segment of the target nucleic acid and an effector protein that exhibits sequence independent cleavage upon forming a complex comprising the segment of the guide nucleic acid binding to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample.

Methods may comprise contacting the sample comprising the target nucleic acid with a guide nucleic acid targeting a target nucleic acid segment, an effector protein capable of being activated when complexed with the guide nucleic acid and the target nucleic acid segment, a single stranded nucleic acid of a reporter comprising a detection moiety, wherein the nucleic acid of a reporter is capable of being cleaved by the activated effector protein, thereby generating a first detectable signal, cleaving the single stranded nucleic acid of a reporter using the effector protein that cleaves as measured by a change in color, and measuring the first detectable signal on the support medium.

Methods may comprise contacting the sample or cell with an effector protein or a multimeric complex thereof and a guide nucleic acid at a temperature of at least about 25° C., at least about 30° C., at least about 35° C., at least about 40° C., at least about 50° C., or at least about 65° C. In some embodiments, the temperature is not greater than 80° C. In some embodiments, the temperature is about 25° C., about 30° C., about 35° C., about 40° C., about 45° C., about 50° C., about 55° C., about 60° C., about 65° C., or about 70° C. In some embodiments, the temperature is about 25° C. to about 45° C., about 35° C. to about 55° C., or about 55° C. to about 65° C.

In some embodiments, methods of detecting a target nucleic acid by a cleavage assay. In some embodiments, the target nucleic acid is a single-stranded target nucleic acid. In some embodiments, the cleavage assay comprises: a) contacting the target nucleic acid with a composition comprising an effector protein as described; and b) cleaving the target nucleic acid. In some embodiments, the cleavage assay comprises an assay designed to visualize, quantitate or identify cleavage of a nucleic acid. In some embodiments, the method is an in vitro trans-cleavage assay. In some embodiments, a cleavage activity is a trans-cleavage activity. In some embodiments, the method is an in vitro cis-cleavage assay. In some embodiments, a cleavage activity is a cis-cleavage activity. In some embodiments, the cleavage assay follows a procedure comprising: (i) providing a composition comprising an equimolar amounts of an effector protein as described herein, and a guide nucleic acid described herein, under conditions to form an RNP complex; (ii) adding a plasmid comprising a target nucleic acid, wherein the target nucleic acid is a linear dsDNA, wherein the target nucleic acid comprises a target sequence and a PAM (iii) incubating the mixture under conditions to enable cleavage of the plasmid; (iv) quenching the reaction with EDTA and a protease; and (v) analyzing the reaction products (e.g., viewing the cleaved and uncleaved linear dsDNA with gel electrophoresis).

In some cases, there is a threshold of detection for methods of detecting target nucleic acids. In some embodiments, methods are not capable of detecting target nucleic acids that are present in a sample or solution at a concentration less than or equal to 10 nM. The term “threshold of detection” is used herein to describe the minimal amount of target nucleic acid that must be present in a sample in order for detection to occur. For example, when a threshold of detection is 10 nM, then a signal can be detected when a target nucleic acid is present in the sample at a concentration of 10 nM or more. In some cases, the threshold of detection is less than or equal to 5 nM, 1 nM, 0.5 nM, 0.1 nM, 0.05 nM, 0.01 nM, 0.005 nM, 0.001 nM, 0.0005 nM, 0.0001 nM, 0.00005 nM, 0.00001 nM, 10 pM, 1 pM, 500 fM, 250 fM, 100 fM, 50 fM, 10 fM, 5 fM, 1 fM, 500 attomole (aM), 100 aM, 50 aM, 10 aM, or 1 aM. In some cases, the threshold of detection is in a range of from 1 aM to 1 nM, 1 aM to 500 pM, 1 aM to 200 pM, 1 aM to 100 pM, 1 aM to 10 pM, 1 aM to 1 pM, 1 aM to 500 fM, 1 aM to 100 fM, 1 aM to 1 fM, 1 aM to 500 aM, 1 aM to 100 aM, 1 aM to 50 aM, 1 aM to 10 aM, 10 aM to 1 nM, 10 aM to 500 pM, 10 aM to 200 pM, 10 aM to 100 pM, 10 aM to 10 pM, 10 aM to 1 pM, 10 aM to 500 fM, 10 aM to 100 fM, 10 aM to 1 fM, 10 aM to 500 aM, 10 aM to 100 aM, 10 aM to 50 aM, 100 aM to 1 nM, 100 aM to 500 pM, 100 pM to 200 pM, 100 aM to 100 pM, 100 aM to 10 pM, 100 aM to 1 pM, 100 aM to 500 fM, 100 aM to 100 fM, 100 aM to 1 fM, 100 aM to 500 aM, 500 aM to 1 nM, 500 aM to 500 pM, 500 aM to 200 pM, 500 aM to 100 pM, 500 aM to 10 pM, 500 aM to 1 pM, 500 aM to 500 fM, 500 aM to 100 fM, 500 aM to 1 fM, 1 fM to 1 nM, 1 fM to 500 pM, 1 fM to 200 pM, 1 fM to 100 pM, 1 fM to 10 pM, 1 fM to 1 pM, 10 fM to 1 nM, 10 fM to 500 pM, 10 fM to 200 pM, 10 fM to 100 pM, 10 fM to 10 pM, 10 fM to 1 pM, 500 fM to 1 nM, 500 fM to 500 pM, 500 fM to 200 pM, 500 fM to 100 pM, 500 fM to 10 pM, 500 fM to 1 pM, 800 fM to 1 nM, 800 fM to 500 pM, 800 fM to 200 pM, 800 fM to 100 pM, 800 fM to 10 pM, 800 fM to 1 pM, 1 pM to 1 nM, 1 pM to 500 pM, 1 pM to 200 pM, 1 pM to 100 pM, or 1 pM to 10 pM. In some cases, the threshold of detection in a range of from 800 fM to 100 pM, 1 pM to 10 pM, 10 fM to 500 fM, 10 fM to 50 fM, 50 fM to 100 fM, 100 fM to 250 fM, or 250 fM to 500 fM. In some cases, the threshold of detection is in a range of from 2 aM to 100 pM, from 20 aM to 50 pM, from 50 aM to 20 pM, from 200 aM to 5 pM, or from 500 aM to 2 pM.

In some embodiments, the target nucleic acid is present in a cleavage reaction at a concentration of about 10 nM, about 20 nM, about 30 nM, about 40 nM, about 50 nM, about 60 nM, about 70 nM, about 80 nM, about 90 nM, about 100 nM, about 200 nM, about 300 nM, about 400 nM, about 500 nM, about 600 nM, about 700 nM, about 800 nM, about 900 nM, about 1 μM, about 10 μM, or about 100 μM. In some embodiments, the target nucleic acid is present in the cleavage reaction at a concentration of from 10 nM to 20 nM, from 20 nM to 30 nM, from 30 nM to 40 nM, from 40 nM to 50 nM, from 50 nM to 60 nM, from 60 nM to 70 nM, from 70 nM to 80 nM, from 80 nM to 90 nM, from 90 nM to 100 nM, from 100 nM to 200 nM, from 200 nM to 300 nM, from 300 nM to 400 nM, from 400 nM to 500 nM, from 500 nM to 600 nM, from 600 nM to 700 nM, from 700 nM to 800 nM, from 800 nM to 900 nM, from 900 nM to 1 μM, from 1 μM to 10 μM, from 10 μM to 100 μM, from 10 nM to 100 nM, from 10 nM to 1 μM, from 10 nM to 10 μM, from 10 nM to 100 μM, from 100 nM to 1 μM, from 100 nM to 10 μM, from 100 nM to 100 μM, or from 1 μM to 100 μM. In some embodiments, the target nucleic acid is present in the cleavage reaction at a concentration of from 20 nM to 50 μM, from 50 nM to 20 μM, or from 200 nM to 5 μM.

In some cases, methods detect a target nucleic acid in less than 60 minutes. In some cases, methods detect a target nucleic acid in less than about 120 minutes, less than about 110 minutes, less than about 100 minutes, less than about 90 minutes, less than about 80 minutes, less than about 70 minutes, less than about 60 minutes, less than about 55 minutes, less than about 50 minutes, less than about 45 minutes, less than about 40 minutes, less than about 35 minutes, less than about 30 minutes, less than about 25 minutes, less than about 20 minutes, less than about 15 minutes, less than about 10 minutes, less than about 5 minutes, less than about 4 minutes, less than about 3 minutes, less than about 2 minutes, or less than about 1 minute.

Methods may comprise detecting a detectable signal within 5 minutes of contacting the sample and/or the target nucleic acid with the guide nucleic acid and/or the effector protein. In some cases, detecting occurs within 1, 2, 3, 4, 5, 6, 7, 8, 9, 10, 11, 12, 13, 14, 15, 20, 25, 30, 35, 40, 45, 50, 55, 60, 70, 80, 90, 100, 110, or 120 minutes of contacting the target nucleic acid. In some embodiments, detecting occurs within 1 to 120, 5 to 100, 10 to 90, 15 to 80, 20 to 60, or 30 to 45 minutes of contacting the target nucleic acid.

In some embodiments, methods of detecting as disclosed herein are compatible with methods for diagnosis of a disease or disorder. In some embodiments, method of detecting as disclosed herein is compatible with devices and/or kits that may be used for modifying and/or detecting a target nucleic acid. In some embodiments, devices and/or kits comprise components comprising one or more of: compositions described herein; systems described herein; other components or appurtenances as described herein; or combinations thereof. In some embodiments, the devices and/or kits may be used in detection of any one of a disease or disorder associated with a gene selected from a viral genome, a prokaryotic genome, or a eukaryotic genome. In some embodiments, the devices and/or kits may be used in detection of any one of a disease or disorder associated with a non-wild type gene, a gene comprising a non-wild type reading frame, a gene comprising one or more mutations, or abnormal processing upon transcription of a gene. In some embodiments, the devices and/or kits may be used in detection of a modified nucleic acid sequence associated with a disease or disorder associated gene. Also, by way of non-limiting example, the devices and/or kits are compatible with detection of a nucleic acid sequence selected from a viral genome, a prokaryotic genome, or a eukaryotic genome. In some embodiments, the devices are configured to receive a sample, wherein the sample comprises one or more target nucleic acids.

In some embodiments, the methods described herein can further comprise sample preparation. In some embodiments, the method may comprise using a physical filter to filter one or more particles from the sample that do not comprise the at least one analyte of interest (e.g., a target nucleic acid). In some embodiments, the method may comprise lysing the sample before detecting the analyte. In some embodiments, the method may comprise performing enzyme (e.g., Proteinase K or savinase) inactivation on the sample. In some embodiments, the method may comprise performing heat inactivation on the sample. In some embodiments, the method may comprise performing nucleic acid purification on the sample. In some embodiments, the method may comprise contacting a plurality of sub-samples with a plurality of effector protein probes comprising different guide RNAs. In some embodiments, the sample is diluted with a buffer or a fluid or concentrated prior to application to the detection system.

The sample can be prepared before one or more targets are detected within the sample. The sample preparation steps described herein can process a crude sample to generate a pure or purer sample. Sample preparation may comprise one or more physical or chemical processes, including, for example, nucleic acid purification, lysis, binding, washing, and/or eluting. In certain embodiments, sample preparation can comprise the following steps, including sample collection, nucleic acid purification, heat inactivation, enzyme inactivation, and/or base/acid lysis.

In some embodiments, nucleic acid purification can be performed on the sample. Purification can comprise disrupting a biological matrix of a cell to release nucleic acids, denaturing structural proteins associated with the nucleic acids (nucleoproteins), inactivating nucleases that can degrade the isolated product (RNase and/or DNase), and/or removing contaminants (e.g., proteins, carbohydrates, lipids, biological or environmental elements, unwanted nucleic acids, and/or other cellular debris).

In some embodiments, lysis of a collected sample can be performed. Lysis can be performed using a protease (e.g., a Proteinase K or PK enzyme). Exemplary proteases include seine proteases (e.g., Proteinase K, Savinase®, trypsin, Protamex®, etc.), metalloproteinases (e.g., MMP-3, etc.), cysteine proteases (e.g., cathepsin B, papin, etc.), threonine proteases, aspartic proteases (e.g., renin, pepsin, cathepsin D, etc.), glutamic proteases, asparagine peptide lyases, or the like. In some embodiments, a solution of reagents can be used to lyse the cells in the sample and release the nucleic acids so that they are accessible to the effector protein. Active ingredients of the solution can be chaotropic agents, detergents, salts, and can be of high osmolality, ionic strength, and pH. Chaotropic agents or chaotropes are substances that disrupt the three-dimensional structure in macromolecules such as proteins, DNA, or RNA. One example protocol may comprise a 4 M guanidinium isothiocyanate, 25 mM sodium citrate·2H20, 0.5% (w/v) sodium lauryl sarcosinate, and 0.1 M β-mercaptoethanol), but numerous commercial buffers for different cellular targets can also be used. Alkaline buffers can also be used for cells with hard shells, particularly for environmental samples. Detergents such as sodium dodecyl sulphate (SDS) and cetyl trimethylammonium bromide (CTAB) can also be implemented to chemical lysis buffers. Cell lysis can also be performed by physical, mechanical, thermal or enzymatic means, in addition to chemically-induced cell lysis mentioned previously. In some embodiments, depending on the type of sample, nanoscale barbs, nanowires, acoustic generators, integrated lasers, integrated heaters, and/or microcapillary probes can be used to perform lysis.

In some embodiments, heat inactivation can be performed on the sample. In some embodiments, a processed/lysed sample can undergo heat inactivation to inactivate, in the lysed sample, the proteins used during lysing (e.g., a PK enzyme or a lysing reagent) and/or other residual proteins in the sample (e.g., RNases, DNases, viral proteins, etc.). In some embodiments, a heating element integrated into the nucleic acid detection device can be used for heat-inactivation. The heating element can be powered by a battery or another source of thermal or electric energy that is integrated with the nucleic acid detection device.

In some embodiments, enzyme inactivation can be performed on the sample. In some embodiments, a processed/lysed sample can undergo enzyme inactivation to inhibit or inactivate, in the lysed sample, the proteins used during lysing (e.g., a PK enzyme or a lysing reagent) and/or other residual proteins in the sample (e.g., RNases, DNases, etc.). In some embodiments, a solution of reagents can be used to inactivate one or more enzymes present in the sample. Enzyme inactivation can occur before, during, or after lysis, when lysis is performed. For example, an RNase inhibitor may be included as a lysis reagent to inhibit native RNases within the sample (which might otherwise impair target and/or reporter detection downstream). Exemplary RNase inhibitors include RNAse Inhibitor, Murine (NEB), RnaseIn Plus (Promega), Protector Rnase Inhibitor (Roche), SuperaseIn (Ambion), RiboLock (Thermo), Ribosafe (Bioline), or the like. Alternatively, or in combination, when a protease is used for sample lysis, a protease inhibitor can be applied to the lysed sample to inactivate the protease prior to contacting the sample nucleic acids to the effector protein. Additional application of heat may not be required to inhibit the protease (e.g., proteinase K) sufficiently to prevent additional activity of the protease (which could potentially impair effector protein activity downstream, in some embodiments). Exemplary protease inhibitors include AEBSF, antipain, aprotinin, bestatin, chymostatin, EDTA, leupeptin, pepstatin A, phosphoramidon, PMSF, soybean trypsin inhibitor, TPCK, or the like. In some embodiments, enzyme inactivation may occur before, during, after, or instead of heat inactivation. In some embodiments, a target nucleic acid within a sample can undergo amplification before binding to a guide nucleic acid. The target nucleic acid within a purified sample can be amplified.

In some embodiments, the method further comprises reacting the sample liquid with the effector protein, the guide nucleic acid, and the reporter. In some embodiments, the reagents described herein may include a composition for improving detection signal strength, detection reaction time, detection reaction efficiency, stability, solubility, or the like. In some embodiments, the reaction may generate a colorimetric signal, a fluorescent signal, an electrochemical signal, a chemiluminescent signal, or another type of signal. In some embodiments, the reaction may induce color-change in substances.

In some embodiments, the method further comprises detecting a detectable signal when a target nucleic acid is present in the sample. The method can further comprise using an effector protein-based detection module to detect one or more targets (e.g., target sequences or target nucleic acids) in the sample. In some embodiments, the sample can be divided into a plurality of aliquots or subsamples to facilitate sample preparation and to enhance the detection capabilities of the devices of the present disclosure. In some embodiments, the sample is not divided into subsamples. In some embodiments, the detectable signal is a colorimetric signal, a fluorescent signal, an electrochemical signal, a chemiluminescent signal, or another type of signal. In some embodiments, the detectable signal may be a color-change in substances. In some embodiments, detection is achieved using a sensor or detector. In some embodiments, detection is achieved either directly or indirectly. Additional illustrative embodiments for detecting a target nucleic acid using devices described herein are provided herein.

### Amplification of a Target Nucleic Acid

Methods may comprise amplifying a target nucleic acid for detection using any of the compositions or systems described herein. Amplifying may comprise changing the temperature of the amplification reaction, also known as thermal amplification (e.g., PCR). Amplifying may be performed at essentially one temperature, also known as isothermal amplification. Amplifying may improve at least one of sensitivity, specificity, or accuracy of the detection of the target nucleic acid.

In some embodiments, amplification can be accomplished using loop mediated amplification (LAMP), isothermal recombinase polymerase amplification (RPA), and/or polymerase chain reaction (PCR). In some embodiments, digital droplet amplification can used. Such nucleic acid amplification of the sample can improve at least one of a sensitivity, specificity, or accuracy of the detection of the target DNA. The reagents for nucleic acid amplification can comprise a recombinase, an oligonucleotide primer, a single-stranded DNA binding (SSB) protein, and a polymerase. In some embodiments, amplifying may comprise subjecting a target nucleic acid to an amplification reaction selected from transcription mediated amplification (TMA), helicase dependent amplification (HDA), or circular helicase dependent amplification (cHDA), strand displacement amplification (SDA), recombinase polymerase amplification (RPA), loop mediated amplification (LAMP), exponential amplification reaction (EXPAR), rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), and improved multiple displacement amplification (IMDA).

In some embodiments, amplification of the target nucleic acid comprises modifying the sequence of the target nucleic acid. For example, amplification may be used to insert a PAM sequence into a target nucleic acid that lacks a PAM sequence. In some cases, amplification may be used to increase the homogeneity of a target nucleic acid in a sample. For example, amplification may be used to remove a nucleic acid variation that is not of interest in the target nucleic acid sequence.

Amplifying may take 1, 2, 3, 4, 5, 6, 7, 8, 9, 10, 11, 12, 13, 14, 15, 16, 17, 18, 19, 20, 25, 30, 40, 50, or 60 minutes. Amplifying may be performed at a temperature of around 20-45° C. Amplifying may be performed at a temperature of less than about 20° C., less than about 25° C., less than about 30° C., 35° C., less than about 37° C., less than about 40° C., or less than about 45° C. The nucleic acid amplification reaction may be performed at a temperature of at least about 20° C., at least about 25° C., at least about 30° C., at least about 35° C., at least about 37° C., at least about 40° C., or at least about 45° C.

### XI. Methods of Introduction to a Host

A guide nucleic acid (or a nucleic acid comprising a nucleotide sequence encoding same) and/or an effector protein described herein may be introduced into a host cell by any of a variety of well-known methods. As a non-limiting example, a guide nucleic acid and/or effector protein may be combined with a lipid. As another non-limiting example, a guide nucleic acid and/or effector protein may be combined with a particle or formulated into a particle.

### Methods for Introducing System Components and Compositions to a Host

Described herein are methods of introducing various components described herein to a host. A host can be any suitable host, such as a host cell. When described herein, a host cell can be an in vivo or in vitro eukaryotic cell, a prokaryotic cell (e.g., bacterial or archaeal cell), or a cell from a multicellular organism (e.g., a cell line) cultured as a unicellular entity, which eukaryotic or prokaryotic cells can be, or have been, used as recipients for methods of introduction described herein, and include the progeny of the original cell which has been transformed by the methods of introduction described herein. It is understood that the progeny of a single cell may not necessarily be completely identical in morphology or in genomic or total DNA complement as the original parent, due to natural, accidental, or deliberate mutation. A host cell can be a recombinant host cell or a genetically modified host cell, if a heterologous nucleic acid, e.g., an expression vector, has been introduced into the cell.

In certain embodiments, molecules of interest, such as nucleic acids of interest, are introduced to a host. In certain embodiments, polypeptides, such as an effector protein, are introduced to a host. In certain embodiments, vectors, such as lipid particles and/or viral vectors can be introduced to a host. Introduction can be for contact with a host or for assimilation into the host, for example, introduction into a host cell.

In some embodiments, described herein are methods of introducing one or more nucleic acids, such as a nucleic acid encoding an effector protein, a nucleic acid encoding an engineered guide nucleic acid (a nucleic acid that, when transcribed, produces an engineered guide nucleic acid), and/or a donor nucleic acid, or combinations thereof, into a host cell. Any suitable method can be used to introduce a nucleic acid into a cell. Suitable methods include, for example, viral infection, transfection, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct microinjection, nanoparticle-mediated nucleic acid delivery, and the like. Further methods are described throughout.

Introducing one or more nucleic acids into a host cell can occur in any culture media and under any culture conditions that promote the survival of the cells. Introducing one or more nucleic acids into a host cell can be carried out in vivo or ex vivo. Introducing one or more nucleic acids into a host cell can be carried out in vitro.

In some embodiments, an effector protein can be provided as RNA. The RNA can be provided by direct chemical synthesis or may be transcribed in vitro from a DNA (e.g., encoding the effector protein). Once synthesized, the RNA may be introduced into a cell by way of any suitable technique for introducing nucleic acids into cells (e.g., microinjection, electroporation, transfection, etc.). In some embodiments, introduction of one or more nucleic acid can be through the use of a vector and/or a vector system, accordingly, in some embodiments, compositions and system described herein comprise a vector and/or a vector system.

Vectors may be introduced directly to a host. In certain embodiments, host cells can be contacted with one or more vectors as described herein, and in certain embodiments, said vectors are taken up by the cells. Methods for contacting cells with vectors include, but are not limited to, electroporation, calcium chloride transfection, microinjection, lipofection, micro-injection, contact with the cell or particle that comprises a molecule of interest, or a package of cells or particles that comprise molecules of interest.

Components described herein can also be introduced directly to a host. For example, an engineered guide nucleic acid can be introduced to a host, specifically introduced into a host cell. Methods of introducing nucleic acids, such as RNA into cells include, but are not limited to direct injection, transfection, or any other method used for the introduction of nucleic acids.

Polypeptides (e.g., effector proteins) described herein can also be introduced directly to a host. In some embodiments, polypeptides described herein can be modified to promote introduction to a host. For example, polypeptides described herein can be modified to increase the solubility of the polypeptide. Such a polypeptide may optionally be fused to a polypeptide domain that increases solubility. The domain may be linked to the polypeptide through a defined protease cleavage site, such as TEV sequence which is cleaved by TEV protease. The linker may also include one or more flexible sequences, e.g., from 1 to 10 glycine residues. In some embodiments, the cleavage of the polypeptide is performed in a buffer that maintains solubility of the product, e.g., in the presence of from 0.5 to 2 M urea, in the presence of polypeptides and/or polynucleotides that increase solubility, and the like. Domains of interest include endosomolytic domains, e.g., influenza HA domain; and other polypeptides that aid in production, e.g., IF2 domain, GST domain, GRPE domain, and the like. In another example, the polypeptide can be modified to improve stability. For example, the polypeptides may be PEGylated, where the polyethyleneoxy group provides for enhanced lifetime in the blood stream. Polypeptides can also be modified to promote uptake by a host, such as a host cell. For example, a polypeptide described herein can be fused to a polypeptide permeant domain to promote uptake by a host cell. Any suitable permeant domains can be used in the non-integrating polypeptides of the present disclosure, including peptides, peptidomimetics, and non-peptide carriers. Examples include penetratin, a permeant peptide may be derived from the third alpha helix of Drosophila melanogaster transcription factor Antennapaedia; the HIV-1 tat basic region amino acid sequence, e.g., amino acids 49-57 of a naturally-occurring tat protein; and poly-arginine motifs, for example, the region of amino acids 34-56 of HIV-1 rev protein, nonaarginine, octa-arginine, and the like. The site at which the fusion is made may be selected in order to optimize the biological activity, secretion or binding characteristics of the polypeptide. The optimal site can be determined by suitable methods.

### Formulations for Introducing System Components and Compositions to a Host

Described herein are formulations of introducing compositions or components of a system described herein to a host. In some embodiments, such formulations, systems and compositions described herein comprise an effector protein and a carrier (e.g., excipient, diluent, vehicle, or filling agent). In some aspects of the present disclosure, the effector protein is provided in a pharmaceutical composition comprising the effector protein and any pharmaceutically acceptable excipient, carrier, or diluent.

### XII. Methods of Modifying Nucleic Acids

Provided herein are methods of modifying target nucleic acids or the expression thereof. In some embodiments, methods comprise editing a target nucleic acid. In general, editing refers to modifying the nucleobase sequence of a target nucleic acid. Also provided herein are methods of modulating the expression of a target nucleic acid. Fusion effector proteins and systems described herein may be used for such methods. Methods of editing a target nucleic acid may comprise one or more of cleaving the target nucleic acid, deleting one or more nucleotides of the target nucleic acid, inserting one or more nucleotides into the target nucleic acid, modifying one or more nucleotides of the target nucleic acid. Methods of modulating expression of target nucleic acids may comprise modifying the target nucleic acid or a protein associated with the target nucleic acid, e.g., a histone.

In some embodiments, methods comprise contacting a target nucleic acid with a composition described herein. In some embodiments, methods comprise contacting a target nucleic acid with an effector protein described herein. In some embodiments, methods comprise contacting a target nucleic acid with a fusion effector protein described herein. The effector protein may be an effector protein provided in TABLE 1 or a catalytically inactive variant thereof. The effector protein may comprise an amino acid sequence that is at least 70%, at least 75%, at least 80%, at least 85%, at least 90%, at least 95%, at least 97%, at least 98%, at least 99% or 100% identical to a sequence described in TABLE 1. In some embodiments, the amino acid sequence of the effector protein is at least 70%, at least 75%, at least 80%, at least 85%, at least 90%, at least 95%, at least 97%, at least 98%, at least 99% or 100% identical to a sequence described in TABLE 1.

In some embodiments, methods comprise base editing. In some embodiments, base editing comprises contacting a target nucleic acid with a fusion effector protein comprising an effector protein fused to a base editing enzyme, such as a deaminase, thereby changing a nucleobase of the target nucleic acid to an alternative nucleobase. In some embodiments, the nucleobase of the target nucleic acid is adenine (A) and the method comprises changing A to guanine (G). In some embodiments, the nucleobase of the target nucleic acid is cytosine (C) and the method comprises changing C to thymine (T). In some embodiments, the nucleobase of the target nucleic acid is C and the method comprises changing C to G. In some embodiments, the nucleobase of the target nucleic acid is A and the method comprises changing A to G.

In some embodiments, methods introduce a nucleobase change in a target nucleic acid relative to a corresponding wildtype or mutant nucleobase sequence. In some embodiments, methods remove or correct a disease-causing mutation in a nucleic acid sequence, e.g., to produce a corresponding wildtype nucleobase sequence. In some embodiments, methods remove/correct point mutations, deletions, null mutations, or tissue-specific mutations in a target nucleic acid. In some embodiments, methods generate gene knock-out, gene knock-in, gene editing, gene tagging, or a combination thereof. Methods of the disclosure may be targeted to a locus in a genome of a cell.

Modifying at least one gene using the compositions and methods described herein can, in some embodiments, induce a reduction or increase in expression of the one or more genes. In some embodiments, the at least one modified gene results in a reduction in expression, also referred to as gene silencing. In some embodiments, the gene silencing reduces expression of one or more genes by at least 10%, at least 20%, at least 30%, at least 40%, at least 50%, at least 60%, at least 70%, at least 80%, at least 90%, or at least 95%. In some embodiments, gene silencing is accomplished by transcriptional silencing, post-transcriptional silencing, or meiotic silencing. In some embodiments, transcriptional silencing is by genomic imprinting, paramutation, transposon silencing, position effect, or RNA-directed DNA methylation. In some embodiments, post-transcriptional silencing is by RNA interference, RNA silencing, or nonsense mediated decay. In some embodiments, meiotic silencing is by transfection or meiotic silencing of unpaired DNA. In some embodiments, the at least one modified gene results in removing all expression, also referred to as the gene being knocked out (KO). In some embodiments, the compositions, methods or systems increase expression of one or more genes by at least 10%, at least 20%, at least 30%, at least 40%, at least 50%, at least 60%, at least 70%, at least 80%, at least 90%, or at least 100%.

In some embodiments, methods of editing a target nucleic acid or modulating the expression of a target nucleic acid are performed in vivo. In some embodiments, methods of editing a target nucleic acid or modulating the expression of a target nucleic acid are performed in vitro. For example, a plasmid may be modified in vitro using a composition described herein and introduced into a cell or organism. In some embodiments, methods of editing a target nucleic acid or modulating the expression of a target nucleic acid are performed ex vivo. For example, methods may comprise obtaining a cell from a subject, modifying a target nucleic acid in the cell with methods and compositions described herein, and returning the cell to the subject. Methods of editing performed ex vivo may be particularly advantageous to produce CAR T-cells. In some embodiments, methods comprise editing a target nucleic acid or modulating the expression of the target nucleic acid in a cell or a subject. The cell may be a dividing cell. The cell may be a terminally differentiated cell. In some embodiments, the target nucleic acid is a gene.

Methods of editing a target nucleic acid or modulating the expression of a target nucleic acid described herein may be employed to generate a genetically modified cell. The cell may be a prokaryotic cell. The cell may be an archaeal cell. The cell may be a eukaryotic cell. The cell may be a mammalian cell. The cell may be a human cell. The cell may be a T cell. The cell may be a hematopoietic stem cell. The cell may be a bone marrow derived cell, a white blood cell, a blood cell progenitor, or a combination thereof. Generating a genetically modified cell may comprise contacting a target cell with an effector protein or a fusion effector protein described herein and a guide nucleic acid. Contacting may comprise electroporation, acoustic poration, optoporation, viral vector-based delivery, iTOP, nanoparticle delivery (e.g., lipid or gold nanoparticle delivery), cell-penetrating peptide (CPP) delivery, DNA nanostructure delivery, or any combination thereof. In some cases, the nanoparticle delivery comprises lipid nanoparticle delivery or gold nanoparticle delivery. In some cases, the nanoparticle delivery comprises lipid nanoparticle delivery. In some cases, the nanoparticle delivery comprises gold nanoparticle delivery.

Methods may comprise cell line engineering. Generally, cell line engineering comprises modifying a pre-existing cell (e.g., naturally-occurring or engineered) or pre-existing cell line to produce a novel cell line or modified cell line. In some embodiments, modifying the pre-existing cell or cell line comprises contacting the pre-existing cell or cell line with an effector protein or fusion effector protein described herein and a guide nucleic acid. The resulting modified cell line may be useful for production of a protein of interest. Non-limiting examples of cell lines includes: 132-d5 human fetal fibroblasts, 10.1 mouse fibroblasts, 293-T, 3T3, 3T3 Swiss, 3T3-L1, 721, 9L, A-549, A10, A172, A20, A253, A2780, A2780ADR, A2780cis, A375, A431, ALC, ARH-77, B16, B35, BALB/3T3 mouse embryo fibroblast, BC-3, BCP-1 cells, BEAS-2B, BHK-21, BR 293, BS-C-1 monkey kidney epithelial, Bcl-1, bEnd.3, BxPC3, C3H-10T1/2, C6/36, C8161, CCRF-CEM, CHO, CHO Dhfr−/−, CHO-7, CHO-IR, CHO-K1, CHO-K2, CHO-T, CIR, CML T1, CMT, COR-L23, COR-L23/5010, COR-L23/CPR, COR-L23/R23, COS, COS-1, COS-6, COS-7, COS-M6A, COV-434, CT26, CTLL-2, CV1, CaCo2, Cal-27, Calu1, D17, DH82, DLD2, DU145, DuCaP, EL4, EM2, EM3, EMT6/AR1, EMT6/AR10.0, FM3, H1299, H69, HASMC, HB54, HB55, HB56, HCA2, HEK-293, HEKa, HEKn, HL-60, HMEC, HT-29, HUVEC, HeLa, HeLa B, HeLa T4, HeLa-S3, Hep G2, Hepa1c1c7, Huh1, Huh4, Huh7, IC21, J45.01, J82, JY cells, Jurkat, Jurkat, K562 cells, KCL22, KG1, KYO1, Ku812, LNCap, LRMB, MC-38, MCF-10A, MCF-7, MDA-MB-231, MDA-MB-435, MDA-MB-468, MDCK II, MDCK II, MEF, mIMCD-3C8161, MOLT, MONO-MAC 6, MOR/0.2R, MRC5, MTD-1A, Ma-Mel 1-48, MiaPaCell, MyEnd, NALM-1, NCI-H69/CPR, NCI-H69/LX10, NCI-H69/LX20, NCI-H69/LX4, NHDF, NIH-3T3, NRK, NRK-52E, NW-145, OPCN/OPCT cell lines, P388D1, PC-3, PNT-1A/PNT 2, Panel, Peer, RIN-5F, RMA/RMAS, RPTE, Rat6, Raw264.7, RenCa, SEM-K2, SK-UT, SKOV3, SW480, SW620, Saos-2 cells, Sf-9, SkBr3, T-47D, T2, T24, T84, TF1, THP1 cell line, TIB55, U373, U87, U937, VCaP, Vero cells, WEHI-231, WM39, WT-49, X63, YAC-1, and YAR.

In some embodiments, methods described herein comprise CRISPR-Cas9 gene editing techniques. The use of CRISPR-Cas9 gene editing techniques for selection of p53-mutated cells is described in Sinha et al., “A systematic genome-wide mapping of oncogenic mutation selection during CRISPR-Cas9 genome editing”, Nature Comm. 12:6512 (2021), which is hereby incorporated by reference in its entirety. Similarly, the presence of KRAS mutations is described to provide a selective advantage during CRISPR-Cas9 gene editing. Accordingly, in some embodiments, a genome targeted for modification by a composition, system or method described herein comprises a wild-type p53 gene, a wild-type KRAS gene, a mutated p53 gene, a mutated KRAS gene, or any combination thereof. In some embodiments, the genome comprises a p53 mutation and the target nucleic acid comprises WDR48, H2AFX, FANCG, BRIP1, HUS1, XRCC3, PALB2, FANCL, FANCA, FANCC, BRCA1, BRCA2, or any combination thereof. In some embodiments, the genome comprises a wild-type p53 and the target nucleic acid comprises CCNB1, MCM6, ANAPC11, ANAPC10, CDKN1A, or any combination thereof. In some embodiments, the genome comprises a KRAS mutation and the target nucleic acid comprises CRYAA, RTCA, LOR, SLC35B4, EN1, CELA3B, NOG, or any combination thereof.

In some embodiments, the compositions, methods or systems comprise a nucleic acid expression vector, or use thereof, to introduce an effector protein, guide nucleic acid, donor template or any combination thereof to a cell. In some embodiments, the nucleic acid expression vector is a viral vector. Viral vectors include, but are not limited to, retroviruses, adenoviruses, adeno-associated viruses, and herpes simplex viruses. In some embodiments, the viral vector is a replication-defective viral vector, comprising an insertion of a therapeutic gene inserted in genes essential to the lytic cycle, preventing the virus from replicating and exerting cytotoxic effects. In some embodiments, the viral vector is an adeno associated viral (AAV) vector. In some embodiments, the nucleic acid expression vector is a non-viral vector. In some embodiments, compositions and methods comprise a lipid, polymer, nanoparticle, or a combination thereof, or use thereof, to introduce a Cas protein, guide nucleic acid, donor template or any combination thereof to a cell. Non-limiting examples of lipids and polymers are cationic polymers, cationic lipids, or bio-responsive polymers. In some embodiments, the bio-responsive polymer exploits chemical-physical properties of the endosomal environment (e.g., pH) to preferentially release the genetic material in the intracellular space.

Methods of modifying may comprise contacting a target nucleic acid with one or more components, compositions or systems described herein. In some embodiments, a method of modifying comprises contacting a target nucleic acid with at least one of: a) one or more effector proteins, or one or more nucleic acids encoding one or more effector proteins; or b) one or more guide nucleic acids, or one or more nucleic acids encoding one or more guide nucleic acids. In some embodiments, a method of modifying comprises contacting a target nucleic acid with a system described herein wherein the system comprises components comprising at least one of: a) one or more effector proteins, or one or more nucleic acids encoding one or more effector proteins; or b) one or more guide nucleic acids, or one or more nucleic acids encoding one or more guide nucleic acids. In some embodiments, a method of modifying comprises contacting a target nucleic acid with a composition described herein comprising at least one of: a) one or more effector proteins, or one or more nucleic acids encoding one or more effector proteins; or b) one or more guide nucleic acids, or one or more nucleic acids encoding one or more guide nucleic acids; in a composition. In some embodiments, a method of modifying as described herein produces a modified target nucleic acid.

Editing a target nucleic acid sequence may introduce a mutation (e.g., point mutations, deletions) in a target nucleic acid relative to a corresponding wildtype nucleotide sequence. Editing may remove or correct a disease-causing mutation in a nucleic acid sequence to produce a corresponding wildtype nucleotide sequence. Editing a target nucleic acid sequence may remove/correct point mutations, deletions, null mutations, or tissue-specific mutations in a target nucleic acid. Editing a target nucleic acid sequence may be used to generate gene knock-out, gene knock-in, gene editing, gene tagging, or a combination thereof. Methods of the disclosure may be targeted to any locus in a genome of a cell.

Modifying may comprise single stranded cleavage, double stranded cleavage, donor nucleic acid insertion, epigenetic modification (e.g., methylation, demethylation, acetylation, or deacetylation), or a combination thereof. In some embodiments, cleavage (single-stranded or double-stranded) is site-specific, meaning cleavage occurs at a specific site in the target nucleic acid, often within the region of the target nucleic acid that hybridizes with the guide nucleic acid spacer sequence. In some embodiments, the effector proteins introduce a single-stranded break in a target nucleic acid to produce a cleaved nucleic acid. In some embodiments, the effector protein is capable of introducing a break in a single stranded RNA (ssRNA). The effector protein may be coupled to a guide nucleic acid that targets a particular region of interest in the ssRNA. In some embodiments, the target nucleic acid, and the resulting cleaved nucleic acid is contacted with a nucleic acid for homologous recombination (e.g., homology directed repair (HDR)) or non-homologous end joining (NHEJ). In some embodiments, a double-stranded break in the target nucleic acid may be repaired (e.g., by NHEJ or HDR) without insertion of a donor template, such that the repair results in an indel in the target nucleic acid at or near the site of the double-stranded break. In some embodiments, an indel, sometimes referred to as an insertion-deletion or indel mutation, is a type of genetic mutation that results from the insertion and/or deletion of one or more nucleotide in a target nucleic acid. An indel may vary in length (e.g., 1 to 1,000 nucleotides in length) and be detected using methods well known in the art, including sequencing. If the number of nucleotides in the insertion/deletion is not divisible by three, and it occurs in a protein coding region, it is also a frameshift mutation. Indel percentage is the percentage of sequencing reads that show at least one nucleotide has been mutation that results from the insertion and/or deletion of nucleotides regardless of the size of insertion or deletion, or number of nucleotides mutated. For example, if there is at least one nucleotide deletion detected in a given target nucleic acid, it counts towards the percent indel value. As another example, if one copy of the target nucleic acid has one nucleotide deleted, and another copy of the target nucleic acid has 10 nucleotides deleted, they are counted the same. This number reflects the percentage of target nucleic acids that are edited by a given effector protein.

In some embodiments, methods of modifying described herein cleave a target nucleic acid at one or more locations to generate a cleaved target nucleic acid. In some embodiments, the cleaved target nucleic acid undergoes recombination (e.g., NHEJ or HDR). In some embodiments, cleavage in the target nucleic acid may be repaired (e.g., by NHEJ or HDR) without insertion of a donor nucleic acid, such that the repair results in an indel in the target nucleic acid at or near the site of the cleavage site. In some embodiments, cleavage in the target nucleic acid may be repaired (e.g., by NHEJ or HDR) with insertion of a donor nucleic acid, such that the repair results in an indel in the target nucleic acid at or near the site of the cleavage site.

In some embodiments, wherein the compositions, systems, and methods of the present disclosure comprise an additional guide nucleic acid or a use thereof, and such dual-guided compositions, systems, and methods described herein may modify the target nucleic acid in two locations. In some embodiments, dual-guided modifying may comprise cleavage of the target nucleic acid in the two locations targeted by the guide nucleic acids. In some embodiments, upon removal of the sequence between the guide nucleic acids, the wild-type reading frame is restored. A wild-type reading frame may be a reading frame that produces at least a partially, or fully, functional protein. A non-wild-type reading frame may be a reading frame that produces a non-functional or partially non-functional protein.

Accordingly, in some embodiments, compositions, systems, and methods described herein may edit 1 to 1,000 nucleotides or any integer in between, in a target nucleic acid. In some embodiments, 1 to 1,000, 2 to 900, 3 to 800, 4 to 700, 5 to 600, 6 to 500, 7 to 400, 8 to 300, 9 to 200, or 10 to 100 nucleotides, or any integer in between, may be edited by the compositions, systems, and methods described herein. In some embodiments, 1, 2, 3, 4, 5, 6, 7, 8, 9, 10, or more nucleotides may be edited by the compositions, systems, and methods described herein. In some embodiments, 10, 20, 30, 40, 50, 60, 70, 80 90, 100 or more nucleotides, or any integer in between, may be edited by the compositions, systems, and methods described herein. In some embodiments, 100, 200, 300, 400, 500, 600, 700, 800, 900 or more nucleotides, or any integer in between, may be edited by the compositions, systems, and methods described herein.

Methods may comprise use of two or more effector proteins. An illustrative method for introducing a break in a target nucleic acid comprises contacting the target nucleic acid with: (a) a first engineered guide nucleic acid comprising a region that binds to a first effector protein described herein; and (b) a second engineered guide nucleic acid comprising a region that binds to a second effector protein described herein, wherein the first engineered guide nucleic acid comprises an additional region that hybridizes to the target nucleic acid and wherein the second engineered guide nucleic acid comprises an additional region that hybridizes to the target nucleic acid. In some embodiments, the first and second effector protein are identical. In some embodiments, the first and second effector protein are not identical.

In some embodiments, editing a target nucleic acid comprises genome editing. Genome editing may comprise editing a genome, chromosome, plasmid, or other genetic material of a cell or organism. In some embodiments, the genome, chromosome, plasmid, or other genetic material of the cell or organism is modified in vivo. In some embodiments, the genome, chromosome, plasmid, or other genetic material of the cell or organism is modified in a cell. In some embodiments, the genome, chromosome, plasmid, or other genetic material of the cell or organism is modified in vitro. For example, a plasmid may be edited in vitro using a composition described herein and introduced into a cell or organism.

In some embodiments, editing a target nucleic acid may comprise deleting a sequence from a target nucleic acid. For example, a mutated sequence or a sequence associated with a disease may be removed from a target nucleic acid. In some embodiments, editing a target nucleic acid may comprise replacing a sequence in a target nucleic acid with a second sequence. For example, a mutated sequence or a sequence associated with a disease may be replaced with a second sequence lacking the mutation or that is not associated with the disease. In some embodiments, editing a target nucleic acid may comprise deleting or replacing a sequence comprising markers associated with a disease or disorder. In some embodiments, editing a target nucleic acid may comprise introducing a sequence into a target nucleic acid. For example, a beneficial sequence or a sequence that may reduce or eliminate a disease may be inserted into the target nucleic acid.

In some embodiments, methods comprise inserting a donor nucleic acid into a cleaved target nucleic acid. The donor nucleic acid may be inserted at a specified (e.g., effector protein targeted) point within the target nucleic acid. In some embodiments, the cleaved target nucleic acid is cleaved at a single location. In such embodiments, the methods comprise contacting a target nucleic acid with an effector protein described herein, thereby introducing a single-stranded break in the target nucleic acid; and contacting the target nucleic acid with a donor nucleic acid for homologous recombination, optionally by HDR or NHEJ, thereby introducing a new sequence into the target nucleic acid (e.g., at a cleavage site). In some embodiments, the cleaved target nucleic acid is cleaved at two locations. In such embodiments, the methods comprise contacting a target nucleic acid with an effector protein described herein, thereby introducing a single-stranded break in the target nucleic acid; contacting the target nucleic acid with a second effector protein described herein, to generate a second cleavage site in the target nucleic acid, ligating the regions flanking the first and second cleavage site, optionally through NHEJ or single-strand annealing, thereby resulting in the excision of a portion of the target nucleic acid between the first and second cleavage sites from the target nucleic acid; and contacting the target nucleic acid with a donor nucleic acid for homologous recombination, optionally by HDR or NHEJ, thereby introducing a new sequence into the target nucleic acid (e.g., in between two cleavage sites).

In some embodiments, methods comprise editing a target nucleic acid with two or more effector proteins. Editing a target nucleic acid may comprise introducing a two or more single-stranded breaks in a target nucleic acid. In some embodiments, a break may be introduced by contacting a target nucleic acid with an effector protein and a guide nucleic acid. The guide nucleic acid may bind to the effector protein and hybridize to a region of the target nucleic acid, thereby recruiting the effector protein to the region of the target nucleic acid. Binding of the effector protein to the guide nucleic acid and the region of the target nucleic acid may activate the effector protein, and the effector protein may introduce a break (e.g., a single stranded break) in the region of the target nucleic acid. In some embodiments, editing a target nucleic acid may comprise introducing a first break in a first region of the target nucleic acid and a second break in a second region of the target nucleic acid. For example, editing a target nucleic acid may comprise contacting a target nucleic acid with a first guide nucleic acid that binds to a first effector protein and hybridizes to a first region of the target nucleic acid and a second guide nucleic acid that binds to a second programmable nickase and hybridizes to a second region of the target nucleic acid. The first effector protein may introduce a first break in a first strand at the first region of the target nucleic acid, and the second effector protein may introduce a second break in a second strand at the second region of the target nucleic acid. In some embodiments, a segment of the target nucleic acid between the first break and the second break may be removed, thereby editing the target nucleic acid. In some embodiments, a segment of the target nucleic acid between the first break and the second break may be replaced (e.g., with donor nucleic acid), thereby editing the target nucleic acid.

Methods, systems and compositions described herein may edit a target nucleic acid wherein such editing may effect one or more indels. In some embodiments, where compositions, systems, and/or methods described herein effect one or more indels, the impact on the transcription and/or translation of the target nucleic acid may be predicted depending on: 1) the amount of indels generated; and 2) the location of the indel on the target nucleic acid. For example, as described herein, in some embodiments, if the amount of indels is not divisible by three, and the indels occur within or along a protein coding region, then the edit or mutation may be a frameshift mutation. In some embodiments, if the amount of indels is divisible by three, then a frameshift mutation may not be effected, but a splicing disruption mutation and/or sequence skip mutation may be effected, such as an exon skip mutation. In some embodiments, if the amount of indels is not evenly divisible by three, then a frameshift mutation may be effected.

Methods, systems and compositions described herein may edit a target nucleic acid wherein such editing may be measured by indel activity. Indel activity measures the amount of change in a target nucleic acid (e.g., nucleotide deletion(s) and/or insertion(s)) compared to a target nucleic acid that has not been contacted by a polypeptide described in compositions, systems, and methods described herein. For example, indel activity may be detected by next generation sequencing of one or more target loci of a target nucleic acid where indel percentage is calculated as the fraction of sequencing reads containing insertions or deletions relative to an unedited reference sequence. In some embodiments, methods, systems, and compositions comprising an effector protein and guide nucleic acid described herein may exhibit about 0.0001% to about 65% or more indel activity upon contact to a target nucleic acid compared to a target nucleic acid non-contacted with compositions, systems, or by methods described herein. For example, methods, systems, and compositions comprising an effector protein and guide nucleic acid described herein may exhibit about 0.0001%, about 0.001%, about 0.01%, about 0.1%, about 1%, about 5%, about 10%, about 15%, about 20%, about 25%, about 30%, about 35%, about 40%, about 45%, about 50%, about 55%, about 60%, about 65% or more indel activity.

In some embodiments, editing of a target nucleic acid as described herein effects one or more mutations comprising splicing disruption mutations, frameshift mutations (e.g., 1+ or 2+ frameshift mutation), sequence deletion, sequence skipping, sequence reframing, sequence knock-in, or any combination thereof. In some embodiments, the splicing disruption can be an editing that disrupts a splicing of a target nucleic acid or a splicing of a sequence that is transcribed from a target nucleic acid relative to a target nucleic acid without the splicing disruption. In some embodiments, the frameshift mutation can be an editing that alters the reading frame of a target nucleic acid relative to a target nucleic acid without the frameshift mutation. In some embodiments, the frameshift mutation can be a +2 frameshift mutation, wherein a reading frame is edited by 2 bases. In some embodiments, the frameshift mutation can be a +1 frameshift mutation, wherein a reading frame is edited by 1 base. In some embodiments, the frameshift mutation is an editing that alters the number of bases in a target nucleic acid so that it is not divisible by three. In some embodiments, the frameshift mutation can be an editing that is not a splicing disruption. In some embodiments a sequence as described in reference to the sequence deletion, sequence skipping, sequence refraining, and sequence knock-in can be a DNA sequence, a RNA sequence, an edited DNA or RNA sequence, a mutated sequence, a wild-type sequence, a coding sequence, a non-coding sequence, an exonic sequence (exon), an intronic sequence (intron), or any combination thereof. In some embodiments, the sequence deletion is an editing where one or more sequences in a target nucleic acid are deleted relative to a target nucleic acid without the sequence deletion. In some embodiments, the sequence deletion can result in or effect a splicing disruption or a frameshift mutation. In some embodiments, the sequence deletion result in or effect a splicing disruption. In some embodiments, the sequence skipping is an editing where one or more sequences in a target nucleic acid are skipped upon transcription or translation of the target nucleic acid relative to a target nucleic acid without the sequence skipping. In some embodiments, the sequence skipping can result in or effect a splicing disruption or a frameshift mutation. In some embodiments, the sequence skipping can result in or effect a splicing disruption. In some embodiments, the sequence reframing is an editing where one or more bases in a target are edited so that the reading frame of the sequence is reframed relative to a target nucleic acid without the sequence reframing. In some embodiments, the sequence reframing can result in or effect a splicing disruption or a frameshift mutation. In some embodiments, the sequence reframing can result in or effect a frameshift mutation. In some embodiments, the sequence knock-in is an editing where one or more sequences is inserted into a target nucleic acid relative to a target nucleic acid without the sequence knock-in. In some embodiments, the sequence knock-in can result in or effect a splicing disruption or a frameshift mutation. In some embodiments, the sequence knock-in can result in or effect a splicing disruption.

In some embodiments, editing of a target nucleic acid can be locus specific, wherein compositions, systems, and methods described herein can edit a target nucleic acid at one or more specific loci to effect one or more specific mutations comprising splicing disruption mutations, frameshift mutations, sequence deletion, sequence skipping, sequence refraining, sequence knock-in, or any combination thereof. For example, editing of a specific locus can affect any one of a splicing disruption, frameshift (e.g., 1+ or 2+ frameshift), sequence deletion, sequence skipping, sequence refraining, sequence knock-in, or any combination thereof. In some embodiments, editing of a target nucleic acid can be locus specific, modification specific, or both. In some embodiments, editing of a target nucleic acid can be locus specific, modification specific, or both, wherein compositions, systems, and methods described herein comprise an effector protein described herein and a guide nucleic acid described herein.

Methods of editing a target nucleic acid or modulating the expression of a target nucleic acid may be performed in vivo. Methods of editing a target nucleic acid or modulating the expression of a target nucleic acid may be performed in vitro. For example, a plasmid may be edited in vitro using a composition described herein and introduced into a cell or organism. Methods of editing a target nucleic acid or modulating the expression of a target nucleic acid may be performed ex vivo. For example, methods may comprise obtaining a cell from a subject, editing a target nucleic acid in the cell with methods described herein, and returning the cell to the subject.

In some embodiments, methods of modifying described herein comprise contacting a target nucleic acid with one or more components, compositions or systems described herein. In some embodiments, the one or more components, compositions or systems described herein comprise at least one of: a) one or more effector proteins, or one or more nucleic acids encoding one or more effector proteins; and b) one or more guide nucleic acids, or one or more nucleic acids encoding one or more guide nucleic acids. In some embodiments, the one or more effector proteins introduce a single-stranded break or a double-stranded break in the target nucleic acid.

In some embodiments, methods of modifying described herein comprise using one or more guide nucleic acids or uses thereof, wherein the methods modify a target nucleic acid at a single location. In some embodiments, the methods comprise contacting an RNP comprising an effector protein and a guide nucleic acid to the target nucleic acid. In some embodiments, the methods introduce a mutation (e.g., point mutations, deletions) in the target nucleic acid relative to a corresponding wildtype nucleotide sequence. In some embodiments, the methods remove or correct a disease-causing mutation in a nucleic acid sequence to produce a corresponding wildtype nucleotide sequence. In some embodiments, the methods remove/correct point mutations, deletions, null mutations, or tissue-specific mutations in a target nucleic acid. In some embodiments, the methods introduce a single stranded cleavage, a nick, a deletion of one or two nucleotides, an insertion of one or two nucleotides, a substitution of one or two nucleotides, an epigenetic modification (e.g., methylation, demethylation, acetylation, or deacetylation), or a combination thereof to the target nucleic acid. In some embodiments, the methods comprise using an effector protein and two guide nucleic acids, wherein two RNPs cleave the target nucleic acid at the same location, wherein a first RNP comprises the effector protein and a first guide nucleic acid, and wherein a second RNP comprises the effector protein and a second guide nucleic acid. In some embodiments, methods comprising using two effector protein and two guide nucleic acids, wherein both RNPs cleave the target nucleic acid at the same location, wherein a first RNP comprises a first effector protein and a first target nucleic acid, and wherein a second RNP comprises a second effector protein and a second target nucleic acid.

In some embodiments, methods of modifying described herein comprise using one or more guide nucleic acids or uses thereof, wherein the methods modify a target nucleic acid at two different locations. In some embodiments, the methods introduce two cleavage sites in the target nucleic acid, wherein a first cleavage site and a second cleavage site comprise one or more nucleotides therebetween. In some embodiments, the methods cause deletion of the one or more nucleotides. In some embodiments, the deletion restores a wild-type reading frame. In some embodiments, the wild-type reading frame produces at least a partially functional protein. In some embodiments, the deletion causes a non-wild-type reading frame. In some embodiments, a non-wild-type reading frame produces a partially functional protein or non-functional protein. In some embodiments, the at least partially functional protein has at least 50%, at least 60%, at least 70%, at least 80%, at least 90%, at least 100%, at least 110%, at least 120%, at least 130%, at least 140%, at least 150%, at least 180%, at least 200%, at least 300%, at least 400% activity compared to a corresponding wildtype protein. In some embodiments, the methods comprise using an effector protein and two guide nucleic acids, wherein two RNPs cleave the target nucleic acid at different locations, wherein a first RNP comprises the effector protein and a first guide nucleic acid, and wherein a second RNP comprises the effector protein and a second guide nucleic acid. In some embodiments, methods comprising using two effector protein and two guide nucleic acids, wherein both RNPs cleave the target nucleic acid at the same location, wherein a first RNP comprises a first effector protein and a first target nucleic acid, and wherein a second RNP comprises a second effector protein and a second target nucleic acid.

In some embodiments, methods of editing described herein comprise inserting a donor nucleic acid into a cleaved target nucleic acid. In some embodiments, the cleaved target nucleic acid formed by introducing a single-stranded break into a target nucleic acid. The donor nucleic acid may be inserted at a specified (e.g., effector protein targeted) point within the target nucleic acid. In some embodiments, the cleaved target nucleic acid is cleaved at a single location. In such embodiments, the methods comprise contacting a target nucleic acid with an effector protein described herein, thereby introducing a single-stranded break in the target nucleic acid; and contacting the target nucleic acid with a donor nucleic acid for homologous recombination, optionally by HDR or NHEJ, thereby introducing a new sequence into the target nucleic acid (e.g., at a cleavage site). In some embodiments, the cleaved target nucleic acid is cleaved at two locations. In such embodiments, the methods comprise contacting a target nucleic acid with an effector protein described herein, thereby introducing a single-stranded break in the target nucleic acid; contacting the target nucleic acid with a second effector protein described herein, to generate a second cleavage site in the target nucleic acid, ligating the regions flanking the first and second cleavage site, optionally through NHEJ or single-strand annealing, thereby resulting in the excision of a portion of the target nucleic acid between the first and second cleavage sites from the target nucleic acid; and contacting the target nucleic acid with a donor nucleic acid for homologous recombination, optionally by HDR or NHEJ, thereby introducing a new sequence into the target nucleic acid (e.g., in between two cleavage sites).

### Donor Nucleic Acids

In some embodiments, methods comprise contacting a target nucleic acid with a donor nucleic acid. In some embodiments, composition described herein comprise a donor nucleic acid. In some embodiments, a donor nucleic acid comprises a sequence that is derived from a plant, bacteria, fungi, virus, or an animal. In some embodiments, the animal is a non-human animal, such as, by way of non-limiting example, a mouse, rat, hamster, rabbit, pig, bovine, deer, sheep, goat, chicken, cat, dog, ferret, a bird, non-human primate (e.g., marmoset, rhesus monkey). In some embodiments, the non-human animal is a domesticated mammal or an agricultural mammal. In some embodiments, the animal is a human. In some embodiments, the sequence comprises a human wild-type (WT) gene or a portion thereof. In some embodiments, the human WT gene or the portion thereof comprises a nucleotide sequence that is at least 70%, at least 80%, at least 90%, at least 92%, at least 95%, at least 97%, at least 99%, or 100% identical to an equal length portion of the WT sequence of any one of the sequences recited in TABLE 8. In some embodiments, the donor nucleic acid is incorporated into an insertion site of a target nucleic acid.

In some embodiments, the donor nucleic acid comprises single-stranded DNA or linear double-stranded DNA. In some embodiments, the donor nucleic acid comprises a nucleotide sequence encoding a functional polypeptide and/or wherein the donor nucleic acid comprises a wildtype sequence. In some embodiments, the donor nucleic acid comprises a protein coding sequence, a gene, a gene fragment, an exon, an intron, an exon fragment, an intron fragment, a gene regulatory fragment, a gene regulatory region fragment, coding sequences thereof, or combinations thereof. In some embodiments, the donor nucleic acid comprises a naturally occurring sequence. In some embodiments, the naturally occurring sequence does not contain a mutation.

In some embodiments, the donor nucleic acid comprises a gene fragment, an exon fragment, an intron fragment, a gene regulatory region fragment, or combinations thereof. In some embodiments, the fragment is at least 10, at least 20, at least 30, at least 40, at least 50, at least 60, at least 70, or at least 80 contiguous nucleotides.

In some embodiments, a donor nucleic acid of any suitable size is integrated into a target nucleic acid or a genome. In some embodiments, the donor nucleic acid integrated into the target nucleic acid or the genome is less than 3, about 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 16, 17, 18, 19, 20, 25, 30, 35, 40, 45, 50, 100, 150, 200, 250, 300, 350, 400, 450, 500 kilobases in length. In some embodiments, the donor nucleic acid is more than 500 kilobases (kb) in length.

Methods may comprise contacting a target nucleic acid, including but not limited to a cell comprising the target nucleic acid, with such compositions. In some embodiments, the donor nucleic acid is inserted at a site that has been cleaved by a composition disclosed herein, for example, an effector protein, resulting in a nick or double strand break.

In reference to a viral vector, the term donor nucleic acid refers to a sequence of nucleotides that will be or has been introduced into a cell following transfection of the viral vector. The donor nucleic acid may be introduced into the cell by any mechanism of the transfecting viral vector, including, but not limited to, integration into the genome of the cell or introduction of an episomal plasmid or viral genome.

In some embodiments, an effector protein as described herein facilitates insertion of a donor nucleic acid at a site of cleavage or between two cleavage sites by cleaving (hydrolysis of a phosphodiester bond) of a nucleic acid resulting in a nick or double strand break—nuclease activity.

In some embodiments, the donor nucleic acid comprises a sequence that serves as a template in the process of homologous recombination. The sequence may carry one or more nucleobase modifications that are to be introduced into the target nucleic acid. By using this donor nucleic acid as a template, the genetic information, including the modification(s), is copied into the target nucleic acid by way of homologous recombination.

### Genetically Modified Cells and Organisms

Methods of editing described herein may be employed to generate a genetically modified cell. The cell may be a eukaryotic cell (e.g., a mammalian cell) or a prokaryotic cell (e.g., an archaeal cell). The cell may be derived from a multicellular organism and cultured as a unicellular entity. The cell may comprise a heritable genetic modification, such that progeny cells derived therefrom comprise the heritable genetic mutation. The cell may be progeny of a genetically modified cell comprising a genetic modification of the genetically modified parent cell. A genetically modified cell may comprise a deletion, insertion, mutation, or non-native sequence relative to a wild-type version of the cell or the organism from which the cell was derived.

In some aspects, disclosed herein are modified cells or populations of modified cells, wherein the modified cell comprises an effector protein described herein, a nucleic acid encoding an effector protein described herein, or a combination thereof. In some embodiments, the modified cell comprises a fusion effector protein described herein, a nucleic acid encoding an effector protein described herein, or a combination thereof. In some embodiments, the modified cell is a modified prokaryotic cell. In some embodiments, the modified cell is a modified eukaryotic cell. A modified cell may be a modified fungal cell. In some embodiments, the modified cell is a modified vertebrate cell. In some embodiments, the modified cell is a modified invertebrate cell. In some embodiments, the modified cell is a modified mammalian cell. In some embodiments, the modified cell is a modified human cell. In some embodiments, the modified cell is in a subject. A modified cell may be in vitro. A modified cell may be in vivo. A modified cell may be ex vivo. A modified cell may be a cell in a cell culture. A modified cell may be a cell obtained from a biological fluid, organ, or tissue of a subject and modified with a composition and/or method described herein. Non-limiting examples of biological fluids are blood, plasma, serum, and cerebrospinal fluid. Non-limiting examples of tissues and organs are bone marrow, adipose tissue, skeletal muscle, smooth muscle, spleen, thymus, brain, lymph node, adrenal gland, prostate gland, intestine, colon, liver, kidney, pancreas, heart, lung, bladder, ovary, uterus, breast, and testes. Non-limiting examples of cells that may be obtained from a subject are hepatocytes, epithelial cells, endothelial cells, neurons, cardiomyocytes, muscle cells and adipocytes.

Methods of the disclosure may be performed in a cell. A cell may be in vitro. A cell may be in vivo. A cell may be ex vivo. A cell may be an isolated cell. A cell may be a cell inside of an organism. A cell may be an organism. A cell may be a cell in a cell culture. A cell may be one of a collection of cells. A cell may be a mammalian cell or derived from a mammalian cell. A cell may be a rodent cell or derived from a rodent cell. A cell may be a human cell or derived from a human cell. A cell may be a prokaryotic cell or derived from a prokaryotic cell. A cell may be a bacterial cell or may be derived from a bacterial cell. A cell may be an archaeal cell or derived from an archaeal cell. A cell may be a eukaryotic cell or derived from a eukaryotic cell. A cell may be a plant cell or derived from a plant cell. A cell may be an animal cell or derived from an animal cell. A cell may be an invertebrate cell or derived from an invertebrate cell. A cell may be a vertebrate cell or derived from a vertebrate cell. A cell may be a microbe cell or derived from a microbe cell. A cell may be a fungi cell or derived from a fungi cell. A cell may be from a specific organ or tissue. In some embodiments, the cell is a progenitor cell or derived therefrom. In some embodiments, the cell is a pluripotent stem cell or derived therefrom. In some embodiments, the cell is from a specific organ or tissue. In some embodiments, the cell is a hepatocyte. In some embodiments, the tissue is a subject's blood, bone marrow, or cord blood. In some embodiments, the tissue is a heterologous donor blood, cord blood, or bone marrow. In some embodiments, the tissue is an allogenic blood, cord blood, or bone marrow. In some embodiments, the tissue may be muscle. In some embodiments, the muscle may be a skeletal muscle. In some embodiments, skeletal muscles include the following: abductor digiti minimi (foot), abductor digiti minimi (hand), abductor hallucis, abductor pollicis brevis, abductor pollicis longus, adductor brevis, adductor hallucis, adductor longus, adductor magnus, adductor pollicis, anconeus, articularis cubiti, articularis genu, aryepiglotticus, auricularis, biceps brachii, biceps femoris, brachialis, brachioradialis, buccinator, bulbospongiosus, constrictor of pharynx-inferior, constrictor of pharynx-middle, constrictor of pharynx-superior, coracobrachialis, corrugator supercilii, cremaster, cricothyroid, dartos, deep transverse perinei, deltoid, depressor anguli oris, depressor labii inferioris, diaphragm, digastric, digastric (anterior view), erector spinae-spinalis, erector spinae-iliocostalis, erector spinae-longissimus, extensor carpi radialis brevis, extensor carpi radialis longus, extensor carpi ulnaris, extensor digiti minimi (hand), extensor digitorum (hand), extensor digitorum brevis (foot), extensor digitorum longus (foot), extensor hallucis brevis, extensor hallucis longus, extensor indicis, extensor pollicis brevis, extensor pollicis longus, external oblique abdominis, flexor carpi radialis, flexor carpi ulnaris, flexor digiti minimi brevis (foot), flexor digiti minimi brevis (hand), flexor digitorum brevis, flexor digitorum longus (foot), flexor digitorum profundus, flexor digitorum superficialis, flexor hallucis brevis, flexor hallucis longus, flexor pollicis brevis, flexor pollicis longus, frontalis, gastrocnemius, gemellus inferior, gemellus superior, genioglossus, geniohyoid, gluteus maximus, gluteus medius, gluteus minimus, gracilis, hyoglossus, iliacus, inferior oblique, inferior rectus, infraspinatus, intercostals external, intercostals innermost, intercostals internal, internal oblique abdominis, interossei-dorsal of hand, interossei-dorsal of foot, interossei-palmar of hand, interossei plantar of foot, interspinales, intertransversarii, intrinsic muscles of tongue, ishiocavernosus, lateral cricoarytenoid, lateral pterygoid, lateral rectus, latissimus dorsi, levator anguli oris, levator ani-coccygeus, levator ani-iliococcygeus, levator ani-pubococcygeus, levator ani-puborectalis, levator ani-pubovaginalis, levator labii superioris, levator labii superioris, alaeque nasi, levator palpebrae superioris, levator scapulae, levator veli palatini, levatores costarum, longus capitis, longus colli, lumbricals of foot, lumbricals of hand, masseter, medial pterygoid, medial rectus, mentalis, m. uvulae, mylohyoid, nasalis, oblique arytenoid, obliquus capitis inferior, obliquus capitis superior, obturator externus, obturator internus (A), obturator internus (B), omohyoid, opponens digiti minimi (hand), opponens pollicis, orbicularis oculi, orbicularis oris, palatoglossus, palatopharyngeus, palmaris brevis, palmaris longus, pectineus, pectoralis major, pectoralis minor, peroneus brevis, peroneus longus, peroneus tertius, piriformis (A), piriformis (B), plantaris, platysma, popliteus, posterior cricoarytenoid, procerus, pronator quadratus, pronator teres, psoas major, psoas minor, pyramidalis, quadratus femoris, quadratus lumborum, quadratus plantae, rectus abdominis, rectus capitus anterior, rectus capitus lateralis, rectus capitus posterior major, rectus capitus posterior minor, rectus femoris, rhomboid major, rhomboid minor, risorius, salpingopharyngeus, sartorius, scalenus anterior, scalenus medius, scalenus minimus, scalenus posterior, semimembranosus, semitendinosus, serratus anterior, serratus posterior inferior, serratus posterior superior, soleus, sphincter ani, sphincter urethrae, splenius capitis, splenius cervicis, stapedius, sternocleidomastoid, sternohyoid, sternothyroid, styloglossus, stylohyoid, stylohyoid (anterior view), stylopharyngeus, subclavius, subcostalis, subscapularis, superficial transverse perinei, superior oblique, superior rectus, supinator, supraspinatus, temporalis, temporoparietalis, tensor fasciae lata, tensor tympani, tensor veli palatini, teres major, teres minor, thyro-arytenoid & vocalis, thyro-epiglotticus, thyrohyoid, tibialis anterior, tibialis posterior, transverse arytenoid, transversospinalis-multifidus, transversospinalis-rotatores, transversospinalis-semispinalis, transversus abdominis, transversus thoracis, trapezius, triceps, vastus intermedius, vastus lateralis, vastus medialis, zygomaticus major, or zygomaticus minor. In some embodiments, the cell is a myocyte. In some embodiments, the cell is a muscle cell. In some embodiments, the muscle cell is a skeletal muscle cell. In some embodiments, the skeletal muscle cell is a red (slow) skeletal muscle cell, a white (fast) skeletal muscle cell or an intermediate skeletal muscle cell.

Non-limiting examples of cells that may be engineered or modified with compositions and methods described herein include stem cells, such as human stem cells, animal stem cells, stem cells that are not derived from human embryonic stem cells, embryonic stem cells, mesenchymal stem cells, pluripotent stem cells, induced pluripotent stem cells (iPS), somatic stem cells, adult stem cells, hematopoietic stem cells, tissue-specific stem cells. A cell may be a pluripotent cell.

Non-limiting examples of cells that may be engineered or modified with compositions and methods described herein include immune cells, such as CART, T-cells, B-cells, NK cells, granulocytes, basophils, eosinophils, neutrophils, mast cells, monocytes, macrophages, dendritic cells, antigen-presenting cells (APC), or adaptive cells.

Non-limiting examples of cells that may be engineered or modified with compositions and methods described herein include plant cells, such as parenchyma, sclerenchyma, collenchyma, xylem, phloem, germline (e.g., pollen). Cells from lycophytes, ferns, gymnosperms, angiosperms, bryophytes, charophytes, chlorophytes, rhodophytes, or glaucophytes.

Methods of the disclosure may be performed in a eukaryotic cell or cell line. Methods may comprise cell line engineering (e.g., engineering a cell from a cell line for bioproduction). In some embodiments, the eukaryotic cell is a Chinese hamster ovary (CHO) cell. Cell lines may be used to produce a desired protein. In some embodiments, target nucleic acids comprise a genomic sequence. In some embodiments, the eukaryotic cell is a Human embryonic kidney 293 cells (also referred to as HEK or HEK 293) cell. Non-limiting examples of cell lines that may be used with compositions, systems and methods of the present disclosure include C8161, CCRF-CEM, MOLT, mIMCD-3, NHDF, HeLa-S3, Huh1, Huh4, Huh7, HUVEC, HASMC, HEKn, HEKa, MiaPaCell, Panel, PC-3, TF1, CTLL-2, CIR, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr −/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepa1c1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, and YAR.

Methods may comprise contacting a cell with a nucleic acid (e.g., a plasmid or mRNA) comprising a nucleobase sequence encoding an effector protein, wherein the effector protein comprise comprises an amino acid sequence that is at least 70%, at least 75%, at least 80%, at least 85%, at least 90%, at least 95%, at least 97%, at least 98%, or at least 99% identical to any one of SEQ ID NOs: 1-78 and 499. Methods may comprise contacting cells with a nucleic acid (e.g., a plasmid or mRNA) comprising a nucleobase sequence encoding a guide nucleic acid, a tracrRNA, a crRNA, or any combination thereof. Contacting may comprise electroporation, acoustic poration, optoporation, viral vector-based delivery, iTOP, nanoparticle delivery (e.g., lipid or gold nanoparticle delivery), cell-penetrating peptide (CPP) delivery, DNA nanostructure delivery, or any combination thereof.

Methods may comprise contacting a cell with an effector protein or a multimeric complex thereof, wherein the effector protein comprises an amino acid sequence that is at least 70%, at least 75%, at least 80%, at least 85%, at least 90%, at least 95%, at least 97%, at least 98%, or at least 99% identical to any one of SEQ ID NOs: 1-78 and 499. Methods may comprise contacting a cell with an effector protein, wherein the amino acid sequence of the effector protein is at least 70%, at least 75%, at least 80%, at least 85%, at least 90%, at least 95%, at least 97%, at least 98%, or at least 99% identical to any one of SEQ ID NOs: 1-78 and 499.

Methods of the disclosure may be performed in a subject. Compositions of the disclosure may be administered to a subject. A subject may be a human. A subject may be a mammal (e.g., rat, mouse, cow, dog, pig, sheep, horse). A subject may be a vertebrate or an invertebrate. A subject may be a laboratory animal. A subject may be a patient. A subject may be suffering from a disease. A subject may display symptoms of a disease. A subject may at risk of developing a disease. A subject may not display symptoms of a disease, but still have a disease. A subject may be under medical care of a caregiver (e.g., the subject is hospitalized and is treated by a physician). In some embodiments, the subject may have a mutation associated with a gene described herein. In some embodiments, the subject may display symptoms associated with a mutation of a gene described herein. Methods of the disclosure may be performed in a plant, bacteria, or a fungus.

### Agricultural Engineering

Compositions and methods of the disclosure may be used for agricultural engineering. For example, compositions and methods of the disclosure may be used to confer desired traits on a plant. A plant may be engineered for the desired physiological and agronomic characteristic using the present disclosure. In some embodiments, the target nucleic acid sequence comprises a nucleic acid sequence of a plant. In some embodiments, the target nucleic acid sequence comprises a genomic nucleic acid sequence of a plant cell. In some embodiments, the target nucleic acid sequence comprises a nucleic acid sequence of an organelle of a plant cell. In some embodiments, the target nucleic acid sequence comprises a nucleic acid sequence of a chloroplast of a plant cell.

The plant may be a dicotyledonous plant. Non-limiting examples of orders of dicotyledonous plants include Magniolales, Illiciales, Laurales, Piperales, Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Dilleniales, Theales, Malvales, Urticales, Lecythidales, Violales, Salicales, Capparales, Ericales, Diapensales, Ebenales, Primulales, Rosales, Fabales, Podostemales, Haloragales, Myrtales, Cornales, Proteales, San tales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, and Asterales.

The plant may be a monocotyledonous plant. Non-limiting examples of orders of monocotyledonous plants include Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, and Orchid ales. A plant may belong to the order, for example, Gymnospermae, Pinales, Ginkgoales, Cycadales, Araucariales, Cupressales and Gnetales.

Non-limiting examples of plants include plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses, wheat, maize, rice, millet, barley, tomato, apple, pear, strawberry, orange, acacia, carrot, potato, sugar beets, yam, lettuce, spinach, sunflower, rape seed, Arabidopsis, alfalfa, amaranth, apple, apricot, artichoke, ash tree, asparagus, avocado, banana, barley, beans, beet, birch, beech, blackberry, blueberry, broccoli, Brussel's sprouts, cabbage, canola, cantaloupe, carrot, cassava, cauliflower, cedar, a cereal, celery, chestnut, cherry, Chinese cabbage, citrus, clementine, clover, coffee, corn, cotton, cowpea, cucumber, cypress, eggplant, elm, endive, eucalyptus, fennel, figs, fir, geranium, grape, grapefruit, groundnuts, ground cherry, gum hemlock, hickory, kale, kiwifruit, kohlrabi, larch, lettuce, leek, lemon, lime, locust, pine, maidenhair, maize, mango, maple, melon, millet, mushroom, mustard, nuts, oak, oats, oil palm, okra, onion, orange, an ornamental plant or flower or tree, papaya, palm, parsley, parsnip, pea, peach, peanut, pear, peat, pepper, persimmon, pigeon pea, pine, pineapple, plantain, plum, pomegranate, potato, pumpkin, radicchio, radish, rapeseed, raspberry, rice, rye, sorghum, safflower, sallow, soybean, spinach, spruce, squash, strawberry, sugar beet, sugarcane, sunflower, sweet potato, sweet corn, tangerine, tea, tobacco, tomato, trees, triticale, turf grasses, turnips, vine, walnut, watercress, watermelon, wheat, yams, yew, and zucchini. A plant may include algae.

### SEQUENCES AND TABLES

TABLE 1 provides illustrative amino acid sequences of effector proteins.

TABLE 1
Effector Proteins

| ID No. | Effector Protein Amino Acid Sequence |
|---|---|
| 2178307 | MRETGKDYYLGLDMGTNSVGWAVTDENYVLMRAKGKDLWGIREFDEALTAVDRR |
| | THRVARRRHQREIARIGLLKEYFHDAIAEVDPDFYQRLDNSKYHEEDKDSAVKGKN |
| | GIFNDANYNDKDYFKQYPTIFHLRKELIESTEKHDVRLVYLALLNMFKHRGHFLNAG |
| | LSTESENTMDTAYHNFVETAAQTIECNFMETVDIEKIKEILGSRDYSRSKKAELVAQI |
| | LHVDSKNKVQMACIKCICGLKVTAAAIFGDKMAADEEKKTDICFSDFGYDEKVPVIL |
| | EIVGEENFELVLAMQEIYDIGSLAGIMKDSLYLSMARVKEYEQHGKDLRILKGVVKK |
| | YGTKEEYDTLFRTMEEGTYSAYVNSVNTKKKSRRDVKKRTRDEFYGRLKKIISKMP |
| | QNDSDVLYIKDKMEKEAFLPKQLTASNGVIPNQIHAREMKKILQNAEIYLPFLKEKDT |
| | SGLTVSERILKLFSFQIPYYVGPVSEKSKLNGGNGWVVRKEEGTVLPWNIEEKIDMTA |
| | TSREFVERMVRRCSYLSDERVLPKGSLLYERFCVLNEINAIKVDGVKIPVEIKQDLYS |
| | KYFKKGKKVTKKKIAEFLIARGVIKEDQTELISGIDIAVNSTLSTYAKFYAIFGEKMEQ |
| | DSYKKMVEQIVEWCTIYGDAKKFLEQKIKECYGNVLTQEEIKRITGFKFRDWGRLSK |
| | EFLELSGCSKATGEVMPLISMMWETNDNLMELLDDNYYTYREALEEKQNNAFKLLA |
| | DIEPEDLDEMYFSAPVKRMVWQTLCIIKELEKVLGKAPKRLFVEMTRNEQKDKKRT |
| | DSRKQQFLDLYKNIKDESRDWKELITRADENGTLKSKKMYLYLIQMGRCMYTGHPI |
| | DLEDLFKDNRYDIDHIYPRHFVKDDNIMNNLVLVEKEKNAHKSDNYPLEEEIYKKQL |
| | PMWKDLHQRKLITDEKFKRLTGRNEFTDEQKAGFIARQLVETSQGTKGVTTILERAL |
| | PDTKIVYAKASNVSEFRQKRDLLKSRVVNDFHHAQDAYLNIVVGNVYFVKFTQNPL |
| | HFIKAEYDKNKAQYEYNLSRMFDRDVRRGNETAWIGCKKGEEAGTILTVKKMMAK |
| | NTPMLTRLSFEGKGGLADQTLYSAEKAKGNGYIPLKTSDSKMQDVTKYGGFNSVST |
| | AYFFLVEHELKGKKVRTLETVPIIWRDRIQKDETMLLKYCTDVLELKNPSIRVKKIKL |
| | QSYIKKDGYYMYITGKTNKQITTKNAVELCVNQRWIDYIKIIEKTVEKKKEECEEIDY |
| | DKNVEFYDLLLTKHTQEIFSNRPNPVGAKLKQGRDKFVSLTLLEQCRVLYEILQLSIIG |
| | NTRADLQLIGGAAISGVMLLSKNITDSKEFILINQSVTGLFEQKVDLLTV (SEQ ID NO: |
| | 1) |
| 2178521 | MAEDQTIGVDIGVASVGWALLNIKKEILESGVCLFPEADAAQNGVRRSMRQSRRLK |
| | RRERTRLGDFEKLWLNSLGKYNRVGNDILGIRNKGLSDSVSMDEIHAVLHYMLKHR |
| | GISYLEDSIDESSGSDYTKGIMVNQKELEKKLPCQIQAERISQYGAFRGNNTVEINGEK |
| | VTLSNTFTTSAYKNELSVFLAKQKEFHPEITDEFVDSYMTIFSRKREYYEGPGNELSR |
| | TDYGKYTLKLDANGNYITENNIFEKLIGKCSVYPEEIRASAASYTAQEFNALEDLNNL |
| | VVNGRKLDEDEKTKIIKVYKSEDTVSVKKIIKKIIGEEIASLSGFRIDKNDKELFHTME |
| | VYRSMRKAMRDRELDIAVFSRDDIDEIARILTINTEKDGIVKAFNESSLDVDNNIVDFF |
| | IEFRKKNGKYFTKWHSFSLKIMNELIPEMYEQSKNQQQLLTDIGYFKRKSDKLKGYD |
| | RVPIDIMTEEIFNPVVKRAVATTVKVINALIDKYGYPGKIVIEMPRDKNSDEEKKRIKD |
| | AQKTNENELKDIIKKVKNEYGIEITDEMFRHHKGLVLKLKLWNEQQGKCLYSGRTIQ |
| | IDELINNQNMYEVDHIIPISISFDDSRANKVLVYATENQLKGNRTPFAYLSTVNREWN |
| | LEQYISYVKLLKFSSKNKIKNLLYAEDITKIDVVKGFINRNINDTSYASRVVLNLLQGF |
| | FAAKEEKTVVSVVRGAFTHQMRVNLNLEKDRDESFNHHAVDAMLIALSQLGYDNY |
| | AARQKAIIDFETGEILSEKEWNDQDNLYRQLLYQAEWYGIKNRIAMAEKSIKYHHRV |
| | EMKCNKGLCNQTIYGTREYNGKTYTIDHLNLYDDSDVARFKKMIEGGKETNFLMYD |
| | NDPETFKALMIVYNQYKDEKNPFVAYHKATDDYLRKYTKSGHNGTIVKKVKFRKEI |
| | TGSCIDISHKYGFDKGSKKVILDTLVPYRMDVYKNRNDGLYYFVGVKQSDIKCMGR |
| | DYVIDEEAYTRTLINERMISEGQSRANLGTLGYEFMISFYKNNIIEYEKGEKIFKERFLS |
| | RTMPNDRNYIETKPVVMQNFGKRNLVGLSKTTSVVKVVTDILGNEKRISKEKFSYIV |
| | DSH (SEQ ID NO: 2) |
| 2178770 | VENVKKYNIGLDIGVASVGWCVTDMESNILKKGNKNMWGSRIFEEAETAANRRSFR |
| | SSKRRLTRRKERIRILQDLLNEDMEKEYPNFFPMLKETSLNYEDKKIDVFVDGKKFNL |
| | FSENGNTDKNYYAKFPTIYHLRKYLINTNEKVDIRLVYLAIHHIIKYRGNFLYEGDFSE |
| | NINAVNEKIQNLIEYLESNYQIIINGKAEDFINILIKKDYSKSRKKELLLQLFNYDKSEK |
| | PIIVNVINAIVGSPFEINKIFDTELEKSKISFSTEIEEIESIKDNLGENLEIFNILNEIYSWYT |
| | LQDILKGKKFISEAFIEKYNKYSDDLELLKKIYKKYFKSEYSSMFRKDGNDNYVAYN |
| | GKSCGKTYKKCAPEAFFNSLKKQMEKLPDNVKEKEIILNGIADNTFLEKINVTENGAI |
| | PYQLHMIELEKIVNNQAKYYKTLEENKNKILQLFSFRIPYYVGPLAKKAEGWAWLV |
| | RKSDDKIMPWNFGDVVDEDATAEEFIKRMTNKCTYLINEDVIPKQSLLYSYFCVLNE |
| | LNNIRINNKHIAKDMKLVVIRDLFQNKKKVTLKMLKDYYKKNAFSCDIVTGLQDGE |
| | NFNSSLTSYIDMKNIFGSVNEQDIEKYEKIIYWITVFEEKKILKRKIKKSFPEITDEQLK |
| | KLLKLNYSGWSRLSKKMLEGLKSYDGDNVIEKLEKTSLNFMQIINSEKFGINKKIEEL |
| | LPVPSKGITYKDIEDIPTSPANKRAIWQSICIVKEIVSIMKTEPSNIYIEFARNEEKKKQM |
| | KDARTKQLLKKYEDINNQLRILKEYDSLVYKQLKKHQSDKILTEKLYLYFIQNGKCL |
| | YSRKPLNIDELDKYEVDHIIPQSYIKDDSLDNKALVIKRENQRKKDSLLLDDKIINDQE |
| | EWWKSLLDAGLISQNKYYKLVRRKMFETEEEKDKFVQRQLVETRQITKYVTNLLIN |
| | EYEKTEIFAIRSNLTHEFREEFKLYKNRNVNNYHHAQDAYILNVIGNIIKKHMYNHNE |
| | FKYSEYVKKHKDENNKNSNVKYSLVMSLIRKYVEQDLVEEVMGYKDCYLSRMLIE |
| | NTGEFYHQTLYSPSQKPVIPLKKGMPVEKYGGYSNENKAYCSIFKYTNQKNKVEYEL |
| | IGIPIQISYEIKQGKITLEEYVSKYYLKEKKFYDLVIVKEKILINQEYLDENNNLMRLCS |
| | DKEIRSSKELIVDKDISEIIYLMNKDEKHLDDEQIEMIEKEYSVVFDYLVKKLDKEYKI |
| | FESTVEKLKTKYKEFENSSSTNKKLIINGLIDLMSTGQGNLKAIGLADREGRKSGQTF |
| | KNEKLLNMTFIDKSITGMYERRYKINGMENCSSK (SEQ ID NO: 3) |
| | LENNKKYNIGLDIGTTSVGWAVVECENQKIIRKGNKALWGVRLFEEANTAADRRNK |
| 2178869 | RSTRRRYDRRRERIRLLQEEFKEEINKIDSNFFTKLKESFYHEDDKENKTIPLTKEEKE |
| | TIKEYNNKYKTIYHLRNKLINSTEKEDIRLVYLAIHHIIKYRGNFLYNSDNFSVENLNL |
| | ETQIETILNSAINFCPSLECPTDYKEYIKINDLSIALLDSSKNDRKVKVQKILESILPKIFV |
| | SEFIKLINGNKFNIIKMIGLENIDKLEISFNGTDYEEKYNELESILGDSIEVLDSMKQLY |
| | DMVFLKRLFKSSESTNISALMVEKYELHKKDLKFLKSILDIDRKIYNKIFKSRKEICPY |
| | ENYIKNKITNEEFVKEIKKYLPSIFEKNINQELLTYYEANIQKRMENGDFLPRITDKDN |
| | GKYPYQLNKEELIKIIENQGKYYPFLLDKTKDDTYKLVKLLEFRIPYYVGPLTEESHS |
| | KFAWMIRKNYNEKITPYNFDDIIDKEATAEKFIKRMISHCTYLLEEPAMPNNSILYSK |
| | YKVMNELKQIRINGKKMSCDMQNKILRNLFMTTSGNITDKKFKEYLYANKDFDMY |
| | QGDFNITGYSADGKFANTMQSYIDFFGENGIFSNTNYTLEEAEQIIEWITIFEDKDILKT |
| | KVERDYPLLNDSQIKKIISKKYSGWGSLCKKLLTTKYYVDKESGVTKSIIDLMEETEN |
| | NFMQIINDDTYNFQKMIKENNKIDVTQKLNYSVVECLATSPSTKRGIYQALRVVDEIV |
| | KYMKKEPEAIMVEMARGDDQKKRKDDRKKYLIKLYEKAKEEIEDYNQLMQQLGKF |
| | EKIDSQKLFLYFIQEGKSLYSGKPLNIEDLATYEIDHIIPRTLIKDDSIDNKALVYREEN |
| | QTKAANYILPREFRSQFNRAWWEHLRKIGLISSKKFHNLIRSEYKEEEIENFINRQLVE |
| | TRQITKHVANILNSYYKNTQVVYLHADLSHNYREKFELFKFRDINDYHHAHDAYLA |
| | AVLGEYKEKYLKCKVNFEMLKELNKSLIEQKDYKKLKYGYVINSLDSSMNDVMKE |
| | VVSIHFDEKTGEVLFDAEEFNRRVEDTLYRNDILISRKPEIKTGQLYKETIYSHNDAKI |
| | KSGIKISKTLPVEKYGFYTNIEYSYLVLVKVKEKNYMFGIPIMYDNKIDKAKYIEEKL |
| | KVSIDDYEIICDRIPFYTKINYHGHLAYLTGSIEFWNAYQLKISKKSMKKWKYMLNFA |
| | LNDKKIPVIDNQEVLTEQEVNKQMLEFIKYLLSLHELFPLYNNTFKKLEKIDIENLDM |
| | EKKKILIKELLKMLSASSMNANLKEFGLTDREGRVHKSNIGEGTLIYNSITGIREKTIVI |
| | EGKRK (SEQ ID NO: 4) |
| 2178891 | MNLKKGKKYNIGLDIGTTSVGWAVVEANTQKVIKKGKAKKALWGVTLFEPASTAE |
| | KTRSFRGTRRRYDRRRERIKLLQNEFASEIDKVDKNFFKKLKESKYHEEDTVNKSIIL |
| | TVDEKKKVKEYNDKYKTIYHLRSRLIHDDLKEDIRLVYLAIHHMVKYRGNFLYNVK |
| | EFNTNNLDLTTTLKNGFDTLVNLVPHLNIIENYEEMISLEEISNAIMMPSKNDIKVKM |
| | KELLDNFSDNKKFGTEFGKLVVGNKFSLKKLLMIEDLEKDISISFDGTDFDDKYNELE |
| | DSLGDLIEVVSVLKELYDTIFLKKLFKGNTISSLSDLMVERYNIHKEDLKFLKQLFNK |
| | ERDLYDQLFRTKTKDKSCFYEQYIHNQLTYEDFIKELKKLLEKISDTNIEDRLRDKYT |
| | KEIQPRIDNGDFLPRITDTENGKYPYQLNKDELIKIIENQGKYYPFLFDKINDDYKIVK |
| | LLEFKIPYYVGPLVSSDKSRFAWMERNIDKVKITPYNFDEVIDKETTAEKFIKRMVSH |
| | CTYLLNEYALPTNSILYSKYKVMNELKQIRINGEKLSKLNEKLPHKIMEELFMKTSGP |
| | ITDKKFKEYLYYSNELTMYENDLIVTGYSSDMKFANNMQPYIDFFGEKGIFENTSYD |
| | LDNADEIIEWITIFEDKDILEKKVRNHYPLLNDFQIKKVLSRKYSGWGSLSKELLTGV |
| | TIKDKKSGVPKNIMTLMEETDENFMQIINNDEYNFQKLIKEKNILKDVDKINYDLVK |
| | DLATSPATKKGIYQALKVIEEIIDYIGYEPESIMIEMARGEDKKKERKDDRKKYLQNL |
| | YQKMKEENRIDDFDDYDVVSQKLNKIEKIDSEKLFLYFIQEGKCLYSGESLNIEDLNS |
| | ADYEVDHILPRTLIKDDSIDNKALVYRKYNQKKAASFVLPSEYRTGYMKKWWNHL |
| | KKNGLISAKKFDRLIRDSYKDEDIQGFIHRQLVETRQITKHVANIISNFYKTTNVIYLK |
| | ANLSHNYRERYELFKFREINDYHHAHDAYLAAVLGEYKEKFMKKDINFAMVKEMN |
| | EKLKALGNYQQLKYGFVINSLDENVSDIVAEISKNIVDKNTGEVLFEAKRFNDQVEK |
| | TLYRNDILVSRKTEIRTGKLFKETIYPKGVGNIPIKENMPTSLYGGYSNVETSYLVLVR |
| | YGKKKKLMGIPLEIELKSKKDINIKYDFIKKHLKSVDDIEIVKDYIPYETLMELKNQK |
| | VYIKGYSISHKNCEVSNALQLKLPSIKAKEWRYVLNKVLNKKNIPMVNGISIVSDELL |
| | IKTMKEIILYLYEQKKNYPLFQGEISKIEEKLNLDEMTLDELEKVIIQLFIIYHCNSANG |
| | NLKDFGLSDRIGRLSGYNIDNGTIISTSTTGIKERDYEL (SEQ ID NO: 5) |
| 2178932 | MGLRFGIDIGVASVGWAVVNDEYEVVESGANLFPSADASKNVDRRTFRQMKRVHR |
| | RRKTRVSDFDKLWKNHGRKLPESNPEEVLELRVKGLTQALTEEELYAVLKNALLHR |
| | GISYLDDAGDEAVSGRSDYERGIMLNRQALDENMHPCEIQFKRLKEYGSYRGNLTID |
| | VDGEKVMLSNVFTTGAYRKEIFEILERQQESHAFLTDDFVENYLKIFSRKREYYVGPG |
| | NEKSRTDYGKYTTRIDAETGQYVTEDNIFEKLIGKCSVYKDLTRAAGATYTAQEFNL |
| | LNDLNNLTVNGRKLEKEEKVQIIEEVKTANTVNMRKILKRVIGEELETLTGARIDKKE |
| | KELFHHFEQYNAIRKAFEKQGWDISELSREDLDSIGDILTLNTERESILGGFQREGLTL |
| | SEEKKDCLIALRKKSGALFSKWQSFSLKIMQELIPELYAQPKNQMQLLTDMGVFKTN |
| | QEKFAEYHQIPDEMILENIYNPVVSRSVRITVKVLNALVKKYGNPSQIVIEMPRDNSE |
| | EEEKKRTKEIQAKNEKELEGIVKRISEEYGIKITEQDFHQHKKLVLKLKLWQEQDGIC |
| | VYSGRAISIQDLLKNPYLFEIDHIIPKSISFDDSRTNKVLVYRTENQEKRNQTPYLYLK |
| | NLRRSWGYDEYRSYVLELKRKGLIPKAKLDKLLFTEDITKIEVLKGFLSRNLNDTRY |
| | ASRMVLNILQSYFKAKKADTTIRVVRGSFTHQLRNVMRLEKDREESYAHHAVDAML |
| | MCYSQMGFDAYHARQKEIIDFETGEILDENKWNQIMSDKAYDELMYQNQWMRIRA |
| | NIQKAEKEVKYWHKRDSKPNRGLCNQTIRGTREKDGATMKVNKLNIYEPSGWAKL |
| | KKMIEQGKQERFLMYLHDRKTWDDMLRIIEEYQDAANPFVEYEKETGDYLRKYAK |
| | KHNGPRIRQLKYLDGEVGSCIDISHKYGHEQGSRKVILESLNPYRMDVYYHAGKKA |
| | YFFVGVKYADLKFEKGRYILNEEAYQRALVSEKLIAASQNRFDLEELGYRFVLSFYR |
| | NEIIEYEKDGEYYTERFLSRTMPKQKNYIETKPMDAPKFEFVDRHFVGLRKTKSVKKI |
| | RLDILGNRFYCSQEKFTLKVDVV (SEQ ID NO: 6) |
| 2178958 | MQIYVFKDIPYSGKLYTDKPETKYTLEEMKMGVRFGLDIGVASVGWAVVNDNYEV |
| | EEAGSNLFTSADANQNVERRSFRQTRRLHRRRGNRINDFKKLWLNTGLPIPATNIPD |
| | MLELRNRGLNGRLSEEEMFHVLKNELLHRGISYLDDATDGSETAQGDYAKGISQNQ |
| | KELDSGKFPCEIQSERLDKYGKYHGNISVGNGEEKIILSNVFTTGAYKKEILAILEQQK |
| | KFLGFLTDSFCEKYIEIFLRKREYYTGPGNEKSRTDYGKYTTRTDETGKYITEDNIFEK |
| | LIGKCSVYKHLTRAAGATYTAQEFNLLNDLNNITVNGRKLSKDEKEVIINEVKNANS |
| | FNMRKILKKAIGEDIETLTGARLDKNEKEDFHKFAQYNAIRKEFEKNGWDITVLSREE |
| | LDSIGNVLTLNTEKEAILNGFKREGLSLTEEMENCLISLRKQKSPLFSKWQSFSLDIML |
| | ELIPGLYEQPKNQMELLTDMGIFKSNKDIFKNYSSIPANAILENIYNPVVTRSIRITIQII |
| | NALIKKYGYPDQIIIEMPRDKNEEEEKKNIAERQKKNQKELDGIIKKISSEYGIKITEQD |
| | FHQHKKLVLKLKLWNEQGGKCPYSGRTINIQDLLYNPSLFEVDHIIPKSISFDDSRSNK |
| | VLVYSTENQKKGNTTPYNYLRHLSGNWGYKEYFDYVCRLRKDNMINKRKMENLLF |
| | TEDINKIEVLKGFITRNINDTRYASRMVLNTLQGYFQAKEAPTKIKVVRGSFTHQFRN |
| | NLYLEKNRDESYSHHAVDAMIMCYSQMGFDEYHKLRSEIIDFETGEILDIKRWEEEV |
| | NEKTYNELIYQNKMIQIRANIQKAEKKVKFWHKRDSKPNRGLCNQTIRGTRNDNGVI |
| | KKVNKLNIYDPSGVETLKKMINNGKQDCFLMYKHDKRTWENMLQIMEDYRETKNP |
| | FTEYENETGDYLRKYSKKHNGPRIVKLKYLDGEVGSCIDISHKYGFETGSRKVILESL |
| | KPYRMDVYYNEKDQKYYLIGVKYSDLKFNKGTYIINEAAYNKTLVSEKLIKPGQTIS |
| | DLERLGYKFMLSFYKNEIIEYEKDGEYYTERFLSRTMPKARNYIETKPLDAPKFQKPQ |
| | KQIGLSKTTSIKKVRLDILGNRYYSGQENFKLTVDIN (SEQ ID NO: 7) |
| 2178992 | MKKYVLGIDIGITSVGYGIIDLETGAFVDYGVRLFKEGTAAENETRRTTRGRRRLTRR |
| | RKTRLSDMRKLLQNNHILNENYHPINNVYELRCKGLHQKLTSDELSAVILHITKHRG |
| | SILETVEDDIEKSKDAESLKATLQKNEQLVKQGNYICEIQLDRLHSQNKIKGHNNNFS |
| | TKDYVNELTEILKHQGLSDDLNKQIINIVKRRRAYYEGPGSEKSPTPYGRFIEVDGQIQ |
| | QIDLIEKMRGKCSIYPDQLRAPKLSVSAELFNLLNDLNNLSVSGEKLTVDEKTMLLKL |
| | ANEKGNLTLKQIAKNLEVEEAEIKGYRIDKNDKPLFTELKGYKKIRNIFKKEGVSVSL |
| | KDYEFIDFIVEILTNKKGIEERKQALSESHFNITEPLADTLANTTGVSGYHSLSLKALR |
| | LLNTELFESTLNQMQLLYELKLFGANKKSYKGKKNIEADIDAILSPVAKRAQNETFK |
| | VINALRKKYGEFDSIVVETTRDKNSDERKKRIKKYQSQNEASSKEMNKMLKESGYD |
| | PEKINGKTRMKIRLYQQQQGKSAYTNQPLELHQVIKDPSYTEIDHIIPISVSLDDSQNN |
| | KVLTLRSENQAKGNLTPFMAFQANKFKYSYAEFKANVLANKNIPYKKRQNLLNEEN |
| | ITKEEVAKKFINRNLNDTSYACRVVFNTLADYFKDNEIDTKVHTVNGRLTDLFRKKI |
| | KLEKERDEDFLHHAVDALIVASIKKMNLLNGYLSKCSFDNLYNETTGEIFTVAGDDK |
| | FFDERYIRYIIDLKTMYLQSNQYYNHLIERKDMIYPPMKISHKINTKPNRKIADETIYS |
| | TRSVDDVDYLIEKIADIYDPKEKKCKTLVEDILNHNEQKYLMAQHDPKTFDLIRQIVE |
| | YHYTQYHDDPKMYKTEIKKEKSEIKLIGDNPLTSYREEHGPIRKYSKKGNGPEICSIK |
| | YQSEKLGNHIDISANYQVDSKKVILKQISPYRTDFYHCSDGKFKFVTVRYINVEYQKS |
| | TQNYVISEKWYEKEKQKKGIKASDTFLFSLHRDELIGIVRQKGKPYYYDLSTEKEGD |
| | KMIADGIRPEILKFTATNNDSTNIIEVKPLYTYCNKRLMCSISNSINLRKYATDVLGNL |
| | YEVKENRLKLEFD (SEQ ID NO: 8) |
| 2179018 | MKDKEYYLGLDMGTASVGWAVTDENYHLIRKKGKDLWGIREFEEALTAVDRRTKR |
| | TSRRSRQRETARIGLLKEYFHDAITQKDEMFYSRLENSKYHIEDKDENVRYKNVLFC |
| | DADYTDKDYFKEFPTIFHLRKALLASPDTHAVRQDIRLVYLALLNMFKHRGHFLNTS |
| | LSEDGGNRRLSEAYTDLRNMLIEELGISFPEANSDQMLDILTNKELSRTRKKEELAKL |
| | LEIERKDKYKMEFVKAICGLKANAKLLFENFEADGNEKIEICFAEYGYEEKAADISAT |
| | LGETYYGVVELLKEIYDICTLANIMKGYSYLSEARVAEYEKHGTDLKILKRTLKRYR |
| | TIKEYNNFFREHKEGTYCAYIGSVNSKKKLRRNIKERKQDDLYKSIKRLLRGLPESDT |
| | DVAYILGELDKGSFLPKQLTPSNGVIPNQVHIKEMKKILENAETYLPFLKERDEYGLTI |
| | SERILKLFSFQIPYYIGPTTEKSKTGWVIRKAPGPILPWNIEEKIDVKKTSEEFVSKMVR |
| | RCSYINGEKVLPKASLLYESFCVLNEINTISIDGERIPVSLKQGIYTALYKKGKKVTRK |
| | QLVNYLRAQGALSDESQLSGIDVTPNNTLSTYGKFKAILGEKIDEDDGKQMVENIIF |
| | WCTIYGDSKRFLRETLSEHYGNVLSAEQIKRIAGIKFKDWGRLSKAFLTLPGCQKSIG |
| | ENTSLIRMMWETNFNLMELLNHPDYSYHEELDARQENGLKPLSQLQAEDLDEFSFSA |
| | PVKRMVWQTLLVIRELENVLGAPPKRLFVEMTRKPDDKKGRTITRKQKFLNLYHNI |
| | DKEIKDWKEEIEKADANGTLKSKKMYLYFTQMGRCMYTGEEIELSQLFNDNIYDIDH |
| | IYPRHFVKDDNLDNNLVLVKKTVNAHKSDIYPLEASIYETQKENWKRLYHANLITEE |
| | KYKRLMGRTPFTDEQKADFIARQLVETRQGTKGVTDILKDLLPNTTIVYSKASNVSE |
| | FRQKRDLIKCRSINEFHHAQDAYLNIVVGNVYYVKFTQNPLHFIQKEYSRNAAQYQY |
| | NLSRMFDRDVVRGNETAWIGSKNDKTPGTIATVKKMMAKNTPLMTRRNFEGHGGI |
| | ADETLYGAQKATAEKYIPLKSSDTKLADVTKYGGFSSVSTAYFFLVEHGAANKRTRT |
| | LETVPIYMKEKINDKEELLLKYCTDVLKLTNPCIRLPKIKLQSLIKKDGYFMHISGKTG |
| | NQIALRNAVNLCLRQEWISYIRKLEKHQDSGGNCLCTEKNIELYLLLLNKHKNSIYA |
| | KRPNPFGEKLAASLEKFKGLALSEQCEVLNQILRLTSIGITCADLTLLGESPNSGKML |
| | MSKNIGKSKELLLLNQSVTGIFESRIDLLTV (SEQ ID NO: 9) |
| 2179042 | MNKKYNLGLDIGTTSVGWAVVESDIQKIIRKGKGKNRKALWGVRLFDEAQTAAGR |
| | RGFRSTRRRYDRRRQRIKLLQEEFKEEIDKVDNTFFQKLQESKYHENDLANKTIILSTS |
| | EKEKIKDYREKYKTIYHLRKELIDDTSKKDIRLVYLAIHHIIKYRGNFLYSGDNFNVD |
| | NLNINSALDEFFEALKNYNQVLNIQENYKEIINYNELVKAMLNPSKNDSKAEIKELFS |
| | ELSSNKNLGSEIGKALVGNKFSVKKLLLLNDVEKDLTISFSDSDYDEKYSELEQTLAE |
| | NIEIIDVLKKIYDTIFLKKMFRGSDLSLISDLMVKRYKTHEIDLKILKKLFAKERILYNK |
| | LLKTKKEMCLYDKYIHNKISYEDFSKELMKLIEKLFDTFEDIDLKEKWLTEIQPRIDTG |
| | DFLPRITDTDNGRYPYQLNLKELQIIITNQGKYYPFLLEQISGKYKIVKLLEFKIPYYVG |
| | PLNNTTSKKGVLNKNAWMIRKQENVKITPYNFDEIINKDVTAERYIKRMISHCTYLLE |
| | EYALPNNSILYSRYKLMNELKQIKINNVRLTIEQQHNILEELFMKTSGTISDRKFKLYL |
| | QISKEFDMYGTEINVTGYSADGKFANSMQSYIDFFGNEGIFEDTSYNEDDAEEIIEWIT |
| | IFEDKDILEKKIRNKYLELNDNQIKKIITKKYSGWGSLSKKLLTELIIKDKRTGTPKNIM |
| | KLMYETEENFMQIINNDEYDFQTLIKNCNINNEIKKVNYKLVEDLATSPSTKKGIYQA |
| | LKVIEEIINYMGYEPERIMIETARGEDTIKVRKDDKKKQLLKLYSKLKEEQELFQLHN |
| | LEKVSKELKNIEKIDSQKLFLYFIQEGKCLYSGKPLNIDDLNTYEIDHILPRTLVKDDSI |
| | DNKALVYRECNQIKGANFVLPQEYRTTYMKNWWSHLKKIGLISASKFYRLTRDKYT |
| | DEDIKGFINRQLVETRQITKHVANIISNLYKNTKVVYLKANLSHNYRERYELFKFREI |
| | NDYHHAHDAYLAAVLGEYKEKYMKKNINFEMIKEMNLRLKNLGNSKQLKYGFVIN |
| | SLDENVNDIVTDISNNLIDDTTGEVLFDAKDFNDRVENTLYRNDILISRKTEIRSGEFY |
| | NQTKQKKGGNGVPLKSNMSIEKYGSYTSLNPSYAVMVKYKKREQICKRLVGMLIYL |
| | EKVSDKERKLYYEKILNLKENSVMDISLKKIPFYSLLCWNGQMGYLVGASSKVEICN |
| | AKQFAYNKDFYKSHKNELNHLFNAKNKENENIDYSEKLSEIIDYIVEKIEKEYKLFEN |
| | LVIELKEIVRHKENFSLSIENKEKIIFQLTKMLNCHSDNANFKFLDSSYSSAFGKKNGR |
| | IIENVSVINLSVTGVKRNQNEF (SEQ ID NO: 10) |
| 2179044 | MKREYNIGLDIGTASVGYAVVDKNTHKVIRKGGKKLLGASLFKPGESAKERRTFRSS |
| | RKRYNRRRQRIKLLQREFKNEIEKVDKDFYQKLKESYYHPKDEMNKTIVISKNEQEK |
| | IKGYHKNYPTIYHLRNKLLTCDKKMDIRLLYLAIHHIIKYRGNFLYTTSDFRIDELNIP |
| | KKQQEIFQDLNQLSDKVTIDEDAIETFPYQELEKALLLSSKTDQSKIVPALFQNILPKEI |
| | AKEMSNLLTGKKASVKKLFQLDLEEDMKVQLEGDSYEQLHDAIESAIGDLIEILEELK |
| | ELYNMIFLKKMFKDDHITSLSALMVKKYEKHKEDLKFLKNILQNHSKEYAEMFKNQ |
| | EKKLCLYEQYVKNKITKEEFRNKTISLVEKIVSKEKKDAIYNDYMTKIKPQLEQEEFL |
| | PRITDTDNGKYPYQLNKEELRIIIEKQSKYYPFLLETTSSGENKLIKLLEFRIPYYVGPL |
| | NDTTDKKDVKNPNAWMIRKIDKRAITPYNFDEVIDKEQTAEEFITRMIGKCTYLLDP |
| | DIKAIAANSILYSKFKVWNELKQIKICENSLSLEMQQKIYKELFLTTKGTITEKKFENY |
| | LKKTGEYKMYDQLTIKGYSADRRFANNMNSYIDFFGEGGFFENTEYREEDAEKIIDW |
| | ITIFEDKEILQSKLEKEYSKLTEKTIKKILNCRYKGWGNLSRELLTNIYYEDQYDGIKK |
| | NIMDLLMETNENFMQIISNKKYGFQEQIDQYNSKHHPSKMNYELVKSLVTSPATKKG |
| | IYIALKVVEELIDYMGEEPNSIIIEMARGEEKKQRKDARKDYLKKLYQNNKNLISDYK |
| | KLMKELNEQDNIDQKLFLYFIQEGKCLYTGTPLEITQLINYEIDHILPRSLYKNDSIDN |
| | KALVLREANQRKAASLVLPAEYRNRNNIAWWKRLTEIGLMSPQKFSKLTRDKYSDK |
| | DIEGFINRQLVETRQITKHVVNIIKNYHKDTNVICIPANLSSHYREKFKLYKFRELNDH |
| | HHAKDAYLAAVLGEYKETYLKKSTDFEKLREQTKKWIDTKKYKELKYGYVINSIDN |
| | EVGQFNQETGEIVFDADEFNKTIAKEMYSNQVLVSRKTEIQTGKFYDETKYKKQSDK |
| | AKGIPIKTNLKVELYGYYQKPQASYAIVVKYEQKGKTKQQMVGIPIYIDKISKPNDQ |
| | KRIDYIKELLKLPKEVNLEIVKDKIPFGITLNVDGEICCLVGASDKIEVCNAKEFYIDKE |
| | NMKKWNQTFHRLFHDAKDAVDNVTYEKHLNEIIEYIIRKIETEFLLYRSSVDTIKEIC |
| | KINNIEKVSIENKEIIIKELLRLLRYNSETANFKLLDKKWNERFGRKSGKTITHAIIINRS |
| | ITGIWEQKDEF (SEQ ID NO: 11) |
| 2179052 | MNEKYVLGLDIGISSVGWGLLRLDEENNPIRIIDTGVKIFSPGEVPKTGASKNLNRRQ |
| | KRGIRRISRRREFRVDRIRYLLNLNGYLGESIVTGKVSEINDELTILYNNMLNDYYKD |
| | KNINPYMLKVEALDRKLSKDELSIILVHYAKHRGYKSNREEDSSDSENGKVKSSIEEN |
| | KKIMEESKYRIISEMFINDNKFKERIRNTNGDYKMCVTREMYLEEIEKVLNAQIKYGL |
| | ITEDFKNEYVEIWSSQRHYAKGPGGDSKYGGNLIEKMTGKCKFDNKPRAPKCAPSSE |
| | IFVALTNLVNFRYKSKRNDEYQNLNSEQIKKIIELAKNQDSITYNKVFKELGIENGIVK |
| | GISATSKEKASAINAFKKKNKLTEFEYGALNEEEKNVYDNLKIEEINKREFIKLKSYSF |
| | MRKEFIKQLGTLKWEEISENLELLDLIAKILTDYKLDDDVEKAIKDNDIDEIYKDAILA |
| | LPNFKDHIMLSSDLVRKINAIMIEGITYDEAMSKLGYSHTDLNSEKEKFDLLIPINQDE |
| | ELNNQRVIRSLSQARGIINSIIKKYGMPERINIETARELAKSREERNEIAKKRDENQEK |
| | NKKIKLELVSLGIFNNIENVKATDVLKYKLWKEQACKCAYSLEAIPIEYLFKDNLVQI |
| | DHILPYSRTFDDTYFNKTLVLSKYNQDKGSKTPYEWLGKTEKWLEYKGFISNLDISE |
| | KKKENYLLLKLTPEIENDFRNQNLNDTKFISKYLVSYIKANLNVSKVGCPSGSITGKL |
| | RNYWNLNGLTHSLDSETYYINNRDENFKKNRENNLHHAMDALVIAATNDKVIRRVS |
| | NYERYKRYLDNKPLEYIIDYGNKIDDNFDVNQFVDENGELQEGSLKDYIKDLRANEY |
| | FIGKHNNTIRTYVPEPYKGFVNEAKIRVYEQNKEIMDFKLKEFNYSDSEINNALPIIPK |
| | FAKDKIGGSLHGETYYSLRVRKSGEDEVKSQISRIDLNSDSFDSKKLENILEKGGGSK |
| | VVYETIKEWIGEEKGSIAYKKNGGYPINPKTGNMIKKIKIENEYKGKGHLLGEKIVDK |
| | ADICQIDVYKKNGEEKLYFVAYDALDLYQIKEKKDKCGNIIKKSQNIDVDIWWGQG |
| | KNHMMMSFSSLNNEYQKYLSLKKNDLVEIELNNGNKGCGYVTGCTSGMIEIKSPLG |
| | DGKDLIGKNLLFQQIRDRYTITISTIKNINKIKISNLGKVEEWDIDKFL (SEQ ID NO: 12) |
| 2179061 | MEKELKKYNIGLDIGTTSVGWAVVEENTQKIIRKGKGNNRKALWGVRLFEEATTAA |
| | ERRGYRSSRRRYDRRRNRIKLLQQEFEEEINKVDKSFYKKLAESKYQSDDKINKSILL |
| | SADEQAKVKKYNDNYKTIYHLRRRLINDKAKEDIRLIYLAIHHIIKYRGNFLNSNENF |
| | NTNNLNLVDHIKGCFEAFINLVPTLNIPENYEEIIDLETLATTIMDNNKNDVKINVENS |
| | LKNISDNKKFSSEFAKIVVGNKFSIKNLFYLESIDKNDEITINFSEADMDNSEKVVKIE |
| | QLLDDKIEVINLLKELYDAIYLKKLFKNSDKASLSELMVARYKTHREDLRFLKDLFD |
| | KERNLYNELFRTKNTKKICLYEKYIHNQISYDEFIKELNKRLEKVFESDIDNALKEKY |
| | TISIKDRIINKEFLPRITDPENGRYPYQLNKDELIKIIENQGKYYPFLLDKVGNKYKLVK |
| | LLEFRIPYYVGPLVSEDKSDFAWMERKIPNVKITPYNFDEVVNKEVTAEKFIKRMISH |
| | CTYLLNEPALPNNSILYSRYKVMNELKQIKINGEKLTQDIQHKIIEELFMKTSGSITDK |
| | KFKIYLHSLNEFSMLGNNINITGYSSDEKFANNMQSYVDFFGENGIFKDTSYNEEDAD |
| | EIIEWITIFDDKDILEKKVKDKYKELNPEKIKMILTKKYSGWGNLSKKLLTELSIKDKK |
| | TNTSKNIMTLLSETEENFMQIINNDEYKFQDLIKKNNVIKETKKINYDLVKDLATSPST |
| | KKGIYQALKVIEELIKYMGYPPTNIMIEMARENNNKKERTLDKKKYLLKLYDKIKND |
| | YKDIYSELKKVDKIDSQKLFLYFIQEGKCLYSGEPLSIENLNEYEIDHILPRTLIKDDSI |
| | DNKALVKRECNQIKAANFVLPEQYRSSYMRKWWNHLKSVNLISAKKFYLLTRSSYN |
| | DKDIQGFINRQLVETRQITKHVANIINNFYKNTKVIYLKANLSHNYRERFELFKFREIN |
| | DYHHAHDAYLAVVLGEYKEKFMKREINFTMVKVMNDKLRSLGNYKQLKYGFVINS |
| | LDENVSEIVTEISKNMIDENTGEVLFDAKTFNEQVEKTLYRNDILISKKTEVRSGEFFN |
| | QTKEPKGGKGVPLKKNMPIKRYGSYTSLKPIYAVLLKYEKKKKYEQKLVGMPIYLA |
| | KSPEDMQTNYFKGLLELEKDSYCEVISKPLPFYSLLNWNNQICYLVGASNKVEVCNA |
| | KEFHYHKNFYKSHKHILNKVFNNNKIEIDETVYDNGLDDVIKYIINKIEQEYVLFDNLI |
| | SELKNIVQYDDMEVLSIEDKENIIIQLTKLLNCKSENANFKLLNSSYSSAFGRKHNKII |
| | DNASVLSLSVTGIKENDYELSDSSNN (SEQ ID NO: 13) |
| 2179107 | MKNYNIGLDIGTTSVGWAVVEEDTQKVIKKGGKRLWGVRLFEEAHVAADRRMKRS |
| | TRRGYDRRRERIRLLQEEFREEILKVDPNFFTKLKESFYNELDVENKTIKISSVEKNLIK |
| | EYHQKFPTIYHLRDRLVHDTSKMDIRLVYLAIHHIVKYRGNFLYHTENFNVEHLDIEK |
| | NLSVLFENLLPILDISLEPDYSMLASAISISSKNDRKLAISECLSGFPKTFVNEFIKVENG |
| | GKFDFVKMLALSMEDAKVKLTFNGSDYEDSYEELEKVLGEYIEILELLRELYDMFFL |
| | KSLFRNSEHTNLSSLMVSKYDTHQKDLKFLKHIFCYDEAIYSKFFKTKKQLCIYEKYV |
| | HNHMTNDEFCREIKKYLPDVFEKVECSSLIDEYTNSVCIRMDNGEFMPRITDGDNSK |
| | YPYQLNKYELIKIIENQGKYYPFLLNKTEDHVEKIVKLLSFRIPYYVGPLVSKEQSQFA |
| | WMIRKDSGKITPYNFDQMIDKEKTAEEFIKRMISHCTYLLNEFAMPACSILYSKYKV |
| | MNELKQIKVNNEGLTNDFQHKVLNQLFMKRSGAITDKVFKEYLYQSREFDMYGTDL |
| | TVTGYSADLKFASNMQSYYDFFGDGGIFEGTSYQEEEAEQIIECITIFEDKEILEKKVK |
| | TNYPELTTEKVGQIVRKKYKGWGSLSRKLLIEKYYVDPTCGTKKSIFDLMYETKKNF |
| | MQILNEDQYQFQKMIDDYNQVDSNQKITYALVENLATAPSIKRGIYQSLLVVEELVK |
| | YMGKDPSSIVIEMARSHESKVRTNDRKKYLTQLYENSKEGIEHYNELMAELQKVEQI |
| | DDRLYLYFMQWGKSLYSQTPLSIENLGEYEIDHIIPRTLIKDDSLDNKVLVLKEENKN |
| | KAASFVVPACYRNSVNQKWWEQLKKMGFLSFKKLHRLSRKYYTDQDIEGFINRQIV |
| | ETRQITKHVANILNHYYPSTKMIYLKSKLLHDYRKRYQLFKFREINNYHHAHDAYLA |
| | AVLGEYKEKHLCRKIDYDIVKTLNAKLFASKDYERLNFGFVINSLDSSFNQVLLPLTK |
| | NVVDVKTGEILLDVDEFHKRVENTLYCNDILISKKVEFRTGEFYQQTKNAKGMSGVS |
| | LKKNLSTKLYGSYTSLHPAYAILVRYTHKGKEEQRLLGLPIYFLEQSKTDTSAISDYV |
| | RRTLKLTDHDTFSIQKERIPFYSLLNWEGQICYLVGASDKVEVCNAKEFYFDREFMK |
| | KYKTTLEKLYHQHRLEVEDYDSNLESVICYIVDKMEKEYKLYENLIVQLKHIVCYDH |
| | LVDLSLEEKEKIIIELLRLLKCDSANANFKFLDSSYSSAFGKKNSRIISSAIIKYHSVTGI |
| | RGRHYEF (SEQ ID NO: 14) |
| 2179109 | MVLRQFHSNIVVGGVSMKNKKKYNIGLDIGTTSVGWAVVESESQKIFRKGNQALWG |
| | VRLFEEANTAAERRAFRSIRRRYDRRRGRIRLLQEEFMTEIDKVDSTFFIKLKESFYHE |
| | SDKNNKTVLLTKREKEEIKEYHERYKTIYHLRKELITSSNKQDIRLVYLALHHMIKYR |
| | GNFLYHVDGFNIENLDLNEQVKIVLNSITNLCSSLGCSEDYDNYLDIKKFTELLLESSK |
| | AKRKEELQKILKNLFPKNFINEFIKLVNGNKFNFIKMLNLEMEESRIELSFDGTDYEEK |
| | YVVFESILGEHMEILTAMKQLYDMIFLKKLFHGSKSISLSSLMVEKYVTHEQDLRFLK |
| | DLFQIDRKIYNKIFRSKKDSCIYEKYIKNKISNDEFIKEIQKYLPEIFDKSGDRKLLDTY |
| | ELEVKNRMENGEFLPRITDTENGKYPYQLNKDELIKIIENQGKYYPFLLDKLEDGTYK |
| | LVKLLEFKIPYYVGPLTDETHSNFAWMIRKNNTERITPYNFDKIVDKEATAEKFIKRM |
| | ISHCTYLLEESAMPNHSILYSKYKVMNELKQIKVNGERLTNNFQQKILKELFMKVSG |
| | GITDKKFKEYLYASKEVTMYQGDISVTGYSADGKFANTMQSYVDFFGENGIFDGTIY |
| | NEENAEEIIEWITIFEDKDILETKIRKKYSKLNEIQIKKIIAKKYHGWGNLSKKLLTTKY |
| | YVEKESGIPKSIMDLMTETKDNFMQIINNEDYHFQKMISDYNKVDTTKKIDYSFVEN |
| | LVTSPSIKRGIYQALKVVEEIVKFMGMQPQAIMIEMARGGAKKERRDDKKKYLTKL |
| | YEKEKDSIHDYYQLMKELGEFEKIDSQKLFLYFIQEGKSLYSGRPLNIEDLDSYEIDHII |
| | PRTLIKDDSIDNKALVYREENQIKAASYILPSKYRTEINIKWWQHLKSINLMSQKKYY |
| | NLIRKQYKTEDIDGFINRQLVETRQITKHVASILGNYYNKSKILYLPVNLSHNYRKRF |
| | DLFKFREINDYHHAHDAYLAAVLGEYKEKYLRKKVNFEMLKELNKKLLEQKDYNK |
| | LKYGYVINSLDYSMNKVLKSMILSESNKEVGELSFDAEGENKRVEDTLYRNDILISRK |
| | TEIRTGEFYNQTKNKKNLPGVCLKKNLPTKLYGSYTSLNPAYAVFVKYTKRGKKEQ |
| | RFIGMPIYIIESSKSDLSIQSNYIRDLLKLNENDVLEFDDRKIPFLSLIDWDGQICYLVG |
| | AADTIEVCNAKQFKFDKNSMKRFKYTLNQVFNVKNRMDTLNEEEYEKNLGELIRYI |
| | VDKMEKEYSLYQNLILNLKNIVCYDYPEMLSIEEKEQSIKELFKLLNCRSTTANFKFL |
| | NSKYSTAFGRKNQRVINHAKVFNKSITGIRESVYEF (SEQ ID NO: 15) |
| 2180100 | MNSYVLGLDIGITSVGYGVIDLKTNNFVDYGVRLFKEGTAAENEARRSARGRRRLLS |
| | RRKNRLEDMRNLLKKYQIIDENFKPLSNVYELRNKGLSTSLTNDELASALLHLTKHR |
| | GSVLDTVEDDEEAAKDSEKTKAVLGTNAKLLAQGRYVCQLQLERLQSEGKVRGHA |
| | NNFKTTDYIEEANEILKHQNIDEKLKEKIIEIIQRKRAYYEGPGSEKSPTPYGRFIEVDG |
| | QIQQIDLIEKMRGKCSVYPNESRAPKMSLSAELFNFLNDLNNLTVDGEKLTTEEKNQI |
| | LKVVSQKGSITLKQVAKLLEVEEDSFAGYRIDKSNKPLLTEFKGYKKVKKIFKECGE |
| | DISFHDYEKIDFIIEIITKQKGMQERKTSLENSKYTFTNTLLDKLVLMNGVSGYHALSF |
| | KALRELNKELYMTDMNQMQILHQMNLFDKNRISHKGRNNIMADETAILSPVARRAQ |
| | NETFKVINELRKKYGEFDSIVIEMTRDKNSVEQAKKQRESQRYFEGLNKEVNRILEEY |
| | GYDADKVNGKTKTKLRLYMEQDGKSAYTLQPLDIRRIISDPTYTEIDHVIPVSISLDDS |
| | LNNKVLITRMENQLKGQMTPVEAYQKGKFVDTGCTLVEYIESVKNNKKYNNKKKG |
| | NLLFKEDITKFSVIQQFINRNLVDTSYACRVVLNTLSDYFKDNGIDTKVHTVVGKLTN |
| | KLRQQIQLEKIRDEDYLHHAVDALIVASIKKMGLLNSYLMKYTFDKLYDEMTGEVK |
| | EVPEDKAFFDEKYISFIVNLKTLYMQSNQYYRGILERKEMVYAPIKISHKINTKPNRQ |
| | VADETIYSTRNIDNKEMIVQKYKDIYEPKFDKLTNDILSGVTEQKYLMARNDPQTFDI |
| | LKQIIMHHFDEFKDSKEYYKKEVKKGVVTYKLVGDNPLYLYKEEFGPVRKCSKKGN |
| | GPVITSMKYVDGQLGSHIDISKNYDVKNKKIILKQVSPYRTDIYLCSDGKYRFVTVRY |
| | KDVFYKKEKQKYVIDANWYAEEKKRKKIDDNAKFICSMHRDELIGIVRKEGDKYIY |
| | DESTEHGGEVKYHDGIHSEILKFTATNNDQNGIIEVKPVFTYCKKQLKPAILNCVEVS |
| | KYATDVLGNLYKVKQNILKLEFE (SEQ ID NO: 16) |
| 2180475 | MADNLKYNIGLDIGTSSVGWCVTDEENNIVKKSGKHLWGSRLFDEGQTAAETRTFR |
| | GTRRRTERRKNRIRYLQSMLLEDIEKVDENFIPRLQQSNLIKDDKNQFKFNLFEDEDFI |
| | DEDYYKQYPTIYHLRKALATEDKKFDIRLVYLALHHIIKYRGNFLTKGEISNETNAIN |
| | LDLENIIEYLKENEIELKYSIGKIKETLVNKELTKSEKAKEILSLFDYGKEDKQIIDNIFK |
| | SVVGLKFNLNAIFNLEDERKLEFASDNDWDSIAGELNENAEIFYSMKNIYSWIIVESFL |
| | DYEDNSDEKEDKTISKAFVIKYDKYHEDLVKLKNAYKKYLDIIQYDKMFRKYEHDN |
| | YYSYNGKLSEIKKSEKCSNEELCERIKEDLKGFEDKSDVSEIFNDISNENYLIKLNTTD |
| | NGEIPYQLHEKELKKILENQSKYHDSINENKDKILSLLTFRIPYYVGPLSTKSKWAWV |
| | KTNSKEAIRPWNFDEIVDKDATAEEFIIRMTNKCTYLLNEDVIPKESLLYAKFSVLNEI |
| | NNIQVNDKRVSKDMKTKIIELFKNKKSVGKKDIKNLYAKDGINAEKIEGFTTSNGKF |
| | NSNMNAYVDFSKIFDNIDENEEMIEELIYWITIFEDKSILKHKIEEKYSSILTKEQINKII |
| | KLKYIGWSRLSKKLLIGLKSTENNETIIEKLMNTNMNFMQIITSDKFGFKEQLEQALK |
| | EVIGEEKKKISYDDIAEIKTSPANKKGIWQAILVVKEITKIMKHDPENIYIEFARSDEEK |
| | GTLTTKKLEKVKKIYDEIENFKQANKALYEDIKKHQGDKNINERLYLYFIQCGKCLY |
| | SDTKLDIEKLSLYQIDHIIPQSYKKDDSIENKALVLSSENQRKKDSYLLTDEIIRKMTPT |
| | WNYLLDKGLISEIKYKKLIKRKMFSNEEEEVNFIARQLVETRQITKHIRGLLSKAYPK |
| | TKIQGIKATITHDFRSKYKIYKIRELNDCHHAQDAYIMCIVGNVLNAADFFEYERMKI |
| | FLKNRKKKTAEERNEQLKEEHGAIMSIIQNKIDINKVKEIINYKDYLVTRKLEEQTGEF |
| | YNQNLLKKIDEPKIKSKTNKDTLKYGGYTNENEAYFVVYEYYNKKGNKEYQLLGIPI |
| | KYSYDIKNGKTTIDEYVKNIAKLQEQTFIRILKEKMLKNQAYLNEFNENELLTSSKEY |
| | KNAKQLYLNTKEIEKMYIVLDSKNKKQLKQEEIEANSIEIYEILLNKIKKEFKTFNTFD |
| | KKKEYEKLDLKDRRIVIINLLRLVNRNHGDLRKIGLSDREGRKRNNSGFNKKKLDNM |
| 2180553 | TFIDQSVTGFYERRYRIDELANSNNKKTM (SEQ ID NO: 17) |
| | MKYDYILGLDIGISSVGWAMLALNEENKPYRIINAGSRIFSPGEVEKTGASRAQARRE |
| | NRGSRRLIRRREFRIDRVRNLLYEEGYLSGNVTSDVVSIKNNELTEIYIKMINNYYREH |
| | ETNPYKLKVEALDRKLSNDELAIILVHYAKKRGYKSNREDKSENDTGKVLSAIDANK |
| | DIMAEKRYRTISEMLIKDEKFSKKIKNSPNNYQMSIENEDYLKEINLVLDTQIKFGTIT |
| | EEFKNKYLEIYNSRRSYAKGPGGDSKYGGNLIERMVGKCKFDNQPRAPKYAYSSELF |
| | VALSKLVNLRYKESDGEYKSLTAPEISKIVDLAKNKTQVTYLDVQKVIGQDIKIKDLS |
| | LNIKEFKKVIENLYKVLDIPSSEKIDIKELNEDDTKKYLTLYNEQLMSKNFIQLKGYH |
| | DLRKAITKSFGKEEWNNISSNISALDDLVLFSTNYKDNTDIKNAIKNSSNLDDKYSDN |
| | NFVELLENYKDHLMLSTDIIRKLIPIMEQGKTYDKAMEELGYNHYNLTENIEKKSLLV |
| | PISKCDNITNQRVIRSLTQSRKVINSIIKKYGTPKVINIETARELAKTMDERNKIAKAQA |
| | ERQGDNERIRQELFEKGFFTDPRKITSTDILKYKLWTEQKEYCPYSLKKITLEDIYVDN |
| | LVQIDHILPYSRTFNDNYLNKTLVFTKYNQEKGNKTPFEWLGNGLKWEEYKSFINSL |
| | TISPLKKENYLLKDLSLDVEREMREQNLNDTKYISKELYSLIKAYLDVPNVNVYQGAI |
| | TAKLRARWGFNRLTHSYISETYNMPSEMKQGIQKDRDNHLHHAMDAIVIASMTKSL |
| | QNKITLYEKFSRYIDGLTKNMLLNLNSDELKETIGEFYDDETGEVKEIDLKEYLKKQF |
| | AANNITFLNHDIGRLKFPVPYETFVEEAKIRVYEQNEEVLKFKLKGLGTYTNEELKKV |
| | HVLTPSFAKSKISGAMHKDTLVGLKQFNNDDSNITYKTSRIPIEKVTPKNLESLIKCNP |
| | SGGTKDIYEAIHEWLGSSSKGEDALKEHNGKYPVSKNDKENKEIKRVKIYEEYKNTG |
| | HNIRNANVDKGDIYKIIIYKSKNKEDNKLYAVGLDIFDIKKIEKYNADKLKNDFNLKL |
| | EYGQGSNFIMKKYSEVLSEYEEYIVLVKNDLIKITCGSKSSIVYVNSCANGQLELKSPL |
| | GDGYDIIGDQNIFNEIVKNYKKTISTIDNIQKLSINTLGEISGL (SEQ ID NO: 18) |
| 2180948 | MKKYNIGLDIGTNSVGWAVVESEKQKVLRKGNKKLWGVRLFDEAVSASSRRNFRS |
| | TRRRYDRRRYRIKLLQEEFQNEINKVDPNFYQIMKESFYNKNDKVNKTIELNKEEKD |
| | FFNSYQKNYPTIYHLRNALINEDKKFDIRLVYLAIHHIIKYRGSFLVQGEFNTESLNIEE |
| | SFHNVVSLICDYCQEFDIDENRDFKDLEKILLLDSKNDKKVELTSYFNFMPKEFTNEFI |
| | KMINGNKFNFIKMFLFDIDSAKIELSFSTTEYDDKYDEYEKILGDRIELLEALKNLYDI |
| | VYLKKIFKESKTSSLSNLMIGYYDKHKENLKFLKQVFNVDRTIGGKKESLYDKLFRT |
| | KNNAKKENYCLYDRYITNGITYDDFQKELKKLFDSVNWNNVDSYLVKKYNEEILMR |
| | LENGDFLPRLTSVENGKFPYQLNKNELVKIIENQKKYYPFLGDEYNGTNKLVQLLEF |
| | RIPYYVGPLTTTEKSNFAWMIRKSGQESIRITPYNFDEVVDKEASAEEFIKKMISTCTY |
| | LLNEKAIPANSILYSKFKVLNELKQIKINDNPLSKELQEQIYNDLFLNTSGTITDKKFKE |
| | YLYANKVTDIYGDDILVKGYSADEKFANSMKSYVDFFGIDGVFENTNYTLNDAEKII |
| | EWITIFEDKNILKVKLNREYPDLNEEKVNKLLKTKYTGWSSLSRKLLTGVLYFDKET |
| | NTSKSIMDLIIETKENFMQILNNKKYKFLELIDKINGEQITKKVNYDLVRDLVTSPATK |
| | RGIYQALKIVDEITNYIGYDPEYISIEMARNEDKKERKPDRKKYLESLYKEYKNKIDN |
| | YNDLNKELKNQEKIDSEKLFLYFIQEGKSLYSMTPLKIENLSDYEVDHIIPRTLIKDDSI |
| | DNKALVLKKENQNKAASFVLPSNYKTDTNRKWWSRLKSIGLISNKKYYNLIRKEFN |
| | SKDIEGFINRQIVETRQITKHVANILANLYENSKVIYLKANLSHNYREKFELYKFRDIN |
| | DYHHAHDAYLAAVLGEYKEKYLRKNIDFDMVRELNKDLIANKDYKSLKYGYVINS |
| | LDDKMNYLINDIIPMQVDEKTGEVITKEFDSKIFNKNIENALYSNDILISKKVEYKSGE |
| | FYNQTKQRKGSKGVSLKENLPTKLYGSYTSLNPAYAIMVTYLNKGKETKKIVGMPIY |
| | VLSTSKKDETKINEYLVNLLNLKNNKDILKISRPIPFYSLLDWNGQICYLVGASDKVE |
| | VVNAKEFKFDKDFFIKHKYTLNKLENNYKAEIDNYDNNLNEIIKYIVDKIEKEYVLYN |
| | NLVSELKSMISYDSLDINSTENKEKIIIELLKLLKCNSANANFKFLNDKFSTAFGKKNG |
| | RIIENVKVKSNSYTGIWSKYNEF (SEQ ID NO: 19) |
| 2181249 | VKNNQSYNIGLDIGVGSVGWCVTDEENNILKKSGKNMWGSRIFSEANTAQERRNFR |
| | SSRRRLDRRKERINILQSLMQQDIENEYPNFLQMLKESSLDFEDKTSSISINGIKYNIFS |
| | NEKYTDQIYYKQFPTIYHLRDYLIKNKEKVDIRLVYLAIHHIIKYRGNFLYEGDFSDD |
| | TTEIIDKIDILTNFLREKYNINLKQDIEKIIDILKQKSMSKANKKDKLISIFEFDKSDKPII |
| | VNVINAILGYAFDLNKIFDINLSKNKISFASDIENEEEIKQQLQDDELIYDCLNETYSWF |
| | ILQDILKGKTYISEAFKEKYYKYAKDLKKLKAIYKEYFPQKYNKMFKSYDKNNYVA |
| | YTGKCSGKTFKKCKAEEFFLKLKKDLDELPDTCVEKEEIKKDIENSNFLRKINVTDNG |
| | AIPHQLHQKELEIILNNQSKYYKTLEENKDNILKLFCFRIPYYVGPLAKDNSNWAWVI |
| | RKNNDEIRPWNFEDVVDEDATAEEFIKRMTNKCTYILNENVIPKQSLLYSRFCVLNEL |
| | NNIRINNHHIVKDIKKLIIERLFKTKKKITRKMLVDLLTKEEIKIESLTGLQDGENFMSN |
| | MQSYIDMEKILGKIDESNIEQCEKIIYWITIFEEKKILKRKIEKEYPKLSLEQINKLAKLK |
| | YTGWSRLSKKLLVGLKATDGESIMEKLEKTSLNFMQIINNDVYGFNKQLEKLMPQK |
| | SGGVQYNDIAEIPTSPANKRAIWQSISIVKEISKIMKQNPKNIFIEFARSEEMNKSMKD |
| | TRVKQLLKKYEEVEKQLKDLKNYDKKVYEELKQHQSDKTLTEKMYLYYIQNGKCL |
| | YSGKALNIDELSQYEVDHILPQSYIKDDSIDNKALVLKEENQRKKDNLLLLDEIINNR |
| | TQWWKSLLENGLISQTKYYRLIKRKMFETDYEREKFVQRQLVETRQITKYVTNLLK |
| | NEYDTSEIYAIRADLTHGFRDKYKIYKNRNINNYHHAHDAYILSVIGNTLNKYWNGT |
| | GEFKYSQYVKKYVEDDKNKYEKNGMILGFINKYIHIEKIKSVMEYKDCFVSRMLEEG |
| | TGAFYNQTLYSPKDNPTIQLKKDKNVLKYGGYSGAQKAYCIIFKYIDKNNKEEYQLI |
| | GVPIQVSYEIKLGKLNLEEYIIDTYLKEKKYSNLKIVKKKILINQEYLDDNYEPMRLCS |
| | DSEIRSNKELVVPHKISELVYLMNLDENRLNDDEKEKLSKGYSIMFNYFLEKLKDEY |
| | KIFESIYLKLIDKMLKFEELKEVDKKSIINGLISLMETGQGNLKSIGLTEREGRKSGQKF |
| | KTDRLSKMIFIDKSITGMYERRYKIGGLENSCS (SEQ ID NO: 20) |
| 2181365 | MEKDYNIGLDIGTTSVGWAVVETNTQKIIRKGKSNHRKALWGVRLFEEATTAAARR |
| | SFRSSRRRYDRRRQRIKLLQEEFKNEMNKVDSDFFRKLVKSKYCEEDKTNKSIILSAD |
| | EKRQVKEYNDKYKTIYHLRNRLISDSSKEDIRLVYLALHHIIKYRGNFIYSGDNFNIDS |
| | LNLSEYLTGVIESLINYIPLFNIPNNYREIIDYKEFESIVLLPSKNDIKVKIKDFFKNIIDN |
| | KNLGSELGKLLTGNKFSVKKLLLLEELENDISISFSEANYDEKYEELSQALGDKIEIIDT |
| | LKQLYDAIFLKKLFKGSDNASISDLMVKRYKAHKEDLKFLKQLFDKNRNLYNKLLR |
| | TKKELCLYDQYIHNKINYEDFIKELVKLVESLFDTLEDNELKEKWLIEIKPRIDSGDFL |
| | PRITDTDNGRYPYQLNLKELNAILENQGKYYPFLLEKVNNKYKIQKILEFKIPYYVGP |
| | LVSEDKSNFAWMERKVNNVKITPYNFDEIVDKASTAEKFIERMISHCTYLLNEYALP |
| | NNSILYSRFKVMNELKQIKVNDRKLEIDLQHKIIEDLFIKTSGTITDKKFKLYLYTLPD |
| | LDMYKGDINITGYSADMKFANNMQSYVDFFGENGIFNNTSYSEENAEEIIKWITIFED |
| | KDILEEKVRSKYTELDDEQIKRIVLKRYSGWGKLSRKLLKELTIKDKESELPKSIIQLM |
| | YETEDNFMQIINKDEYGFQELIKSNNIYLETTRVNYKLVENLATSPATKKGIYQALKV |
| | VEEIIDYMGHEPKNIMIEMARAEEQVKARKDDRKKQLLKLYEKIKETQGELEVSNLR |
| | KVSEELKSIEKIDSQKLFLYFIQEGKCLYSGKPLNIEDLNEYEIDHILPRTLIKDDSIDNK |
| | ALVYRECNQKKADSFVLPENYRTTYMINWWRHLKKNGLISASKFYRLTRKEYNDED |
| | IQGFINRQLIETRQITKHVSNILSNLYKNTKIIYLKANLSHNYRERYELFKFREINDYHH |
| | AHDAYLAAVLGEYKEKYMKKNYNFEMVKEMNHRLRSLGNYRQLNYGFVINSLDE |
| | NVSDIILELSKNLIDEETGEVLFDARKFNERVENTLYRNDILISRKTEIRSGQLFKQTIY |
| | SKGIGNIQLKKNMPTKLYGGYLNVETAYLVLVKYKKKKKLIAIPLEIALKSEHSNEIK |
| | IEFIKKHLNVDNVEIIKDFIPFETLIDYKSQVVYVKGYGVANKVCEVSNAFQLKMPFL |
| | HTKRWKYVLNKVLNKGSIPVVNTKPILSEEEILNVMKEIITYLYEQKSNYPLFENEICK |
| | IEKQLILNEMNYEELAKAIIELLKIYHCNSVNGNLKEFGLGDRIGRLSGKNIDKGIIITK |
| | SITGIKERRYEF (SEQ ID NO: 21) |
| 2181445 | MKNGEYYLGLDMGTSSVGWAVTDDSYQLLKVKGKDLWGIREFEEAKIAVDRRTHR |
| | ISRRRRQRETVRIGILKEYFNDAIMKADPCFYQRLDNSKYCLADKELSVRDKNGIFHD |
| | ANYTDREYYAQYPTIFHLRKELLESDQPHDVRLVYLAVLNMFKHRGHFLNASIGTAS |
| | GERKMSEIYGEFCSLTLEWMDVAFPELDDCSMIENILSMREYSRKRKAEKLADIFHV |
| | SSNEKKKKALVSGICGLKMDLKLLFGDSIEYDADKKMAISFSDAGYEEKTQEILEVL |
| | GDDRFELIQTMKEMYDIGSLASIMKGYTYLSEARVAQYEKHGVDLKILKALVRKYC |
| | DKETYDRFFRKAENGTYSAYVNSTNTGKSLSRDMKGRTREDLYAAIRKLMNHMPA |
| | EDEKVQYVREEMEKESFLPKQLTASNGIIPNQIHLAELKKILENAQGYLLFLQDVDES |
| | GLTVSERIIKLFSFQIPYYLGPLSEHSAENGGNGWVVRRESGQVMPWNLEEKVDVKE |
| | TAEMFITRMVRRCTYMNGENVLPKASLLYEKFCVLNEINNIKVYGEKIPVSVKQDIY |
| | NELFKRGKKVTRKQLYQYLLGRGLVKEGEIEQITGIDITINNYLATYGKFKNILGEGF |
| | DTDDGRNMVEKIVFWCTVYGDFKKFLREQIEEKYGEKLNQEQIKRITGLKFKDWGN |
| | LSKTFLEMQGCSKSTGDILPLISMLWETNDNLMELLAKDAYTYREELEKYQGDSYK |
| | VLAELQAEDLEDMYFSAPVKRMVWQTILIIKEVEKIMGAPPRRLFVEMTRSGEKEKN |
| | RKDSRKQQFLELYKNIKDEEHDWKSIIVDADEKGTLRSKKMFLYLTQRGRSMYTGN |
| | PIDLDDLFNDNLYDIDHIYPRHFVKDDNLANNLVLVEKELNAHKSDTYPLEHDIYTS |
| | QLPMWRELHRQHLISDEKLKRLTGRNPFTDEQKAGFIARQLVETSQGTKGITTILQHA |
| | LPATTIVYAKASNVSEFRQKRDLLKNRTVNDFHHAQDAYLNIVVGNVYFVKFTQNP |
| | LKFIQKEYEKDRDKYNYNLSRMFDWDVKRGDEVAWVAQHGKEESGTIVTVRKMM |
| | EKNTPLLTRLNFEGHGGIAEQTLYSAKKASGVGYIPLKEKDTRMQDVTKYGGFNGV |
| | STAYYILVEHGKTGKRIRTLETVPVLWKDRIEKNLQLLQEYCEKELQLTEPSVRMKKI |
| | KMQSLVKKDGYYMHLSGKTGNQIYVRNAVPLCLKQNWMAYLKELEKGERNEMVS |
| | VEKNMELYDILCGKHSEGIFKRKPTPMGKKLQNGRVVFERLSCEEQGKVLIEMLNLT |
| | KIGEKFANLSLIGDVAKTGVMRIGKKISDAEEFVLINQSVTGVYENRIDLLTV (SEQ ID |
| | NO: 22) |
| 2181509 | MQETGKDYYLGLDMGTNSVGWAVTDENYVLMRAKGKDLWGIREFDEALTAVDRR |
| | THRVARRRRQRETARIGLLKEYFHDAIAEVDPDFYQRLDNSKYHEEDKDSAVKGKN |
| | GIFNDANYNDKDYFKQYPTIFHLRKELIESTGKHDVRLVYLALLNMFKHRGHFLNAG |
| | LSTESENTMDTAYHNFVETAAQTIECNFMETVDIEKIKEILGSRDYSRSKKAELVAQL |
| | LHVDSKNKVQMACIKCICGLKVTAAAIFGDKMAADEEKKTDICFSDFGYDEKVPVIL |
| | EIVGEENFELVLAMQEIYDIGSLAGIMKDSLYLSMARVKEYEQHGKDLRILKGIVKK |
| | YGTKEEYDTLFRTMEEGTYSAYVNSVNTKKKSRRDVKKRTRDEFYGRLKKIISKMP |
| | QDDSDVLYIKDKMEKEAFLPKQLTASNGVIPNQIHAREMKKILQNAEIYLPFLKEKDT |
| | SGLTVSERILKLFSFQIPYYVGPVSEKSKLNGGNGWVVRKEEGTVLPWNIEEKIDMTA |
| | TSREFVERMVRRCSYLSDERVLPKGSLLYERFCVLNEINAIKVDGVKIPVEIKQDLYS |
| | KYFKKGKKVTKKKIAEFLIARGVIKEDQTELISGIDIAVNSTLSTYAKFYAIFGEKMEQ |
| | DSYKKMVEQIVEWCTIYGDAKKFLEQKIKECYGNILTQEEIKRITGFKFRDWGRLSKE |
| | FLELSGCSKATGEIMPLISMMWETNDNLMELLDDNYYTYREALEEKQNNAFKLLAD |
| | IEPEDLDEMYFSAPVKRMVWQTLCIIKELEKVLGKAPKRLFVEMTRNEQKDKKRTDS |
| | RKQQFLDLYKNIKDESRDWKELITRADENGTLKSKKMYLYLTQMGRCMYTGHPIDL |
| | EDLFKDNRYDIDHIYPRHFVKDDNIMNNLVLVEKEKNAHKSDNYPLEEEIYKKQLPM |
| | WKDLHQRKLITDEKFKRLTGRNEFTDEQKAGFIARQLVETSQGTKGVTTILERALPD |
| | TKIVYAKASNVSEFRQKRDLLKSRVVNDFHHAQDAYLNIVVGNVYFVKFTQNPLHFI |
| | KAEYDKNKAQYEYNLSRMFDWDVRRGNETAWIGCKKGEEAGTILTVKKMMAKNT |
| | PMLTRLSFEGKGGLTQKQTLYSAKQAKEDSYMPLKSSDYRMLNVKKYGGFMATST |
| | AYFFLVEYEVDGKKVRTLEMVPNTWKDRIEKNENMLLKYCTEVLKLENPSIRLKKIK |
| | LQSYIKKDGYFMYLSGRSEDRITVKNAVQLCMNSFWMKYIREVEKTVEMKRITEKIS |
| | YEENMKLYKELCEKYNNGIYSKRPTAVGEKLKGAEQAFAKISLYEQCIVISQILALSR |
| | IENPSADLTLIGLSKQTGTMKISKNITNCKEFILINQSVTGLFEQKVDLLTV (SEQ ID |
| | NO: 23) |
| 2181587 | MNERYYVGLDMGTASLGWAVTDEKYRLIRRKGKDLWGVRLFDTANTAAERRTNR |
| | VAKRRLARERAREGYLRMVFAPAIEAVDPGFYQRLDDSKYCEEDKIVKQPFALFAG |
| | NGYTDKDYYEQYPTVFHLRKELLTSKEPHDVRLVFLALLNMYKHRGHFLNSNIGEG |
| | STGSIEEMYENLRSDISQYMELELPDIDDYDKLKNILTSKEYSASRRVELIIESLNLSKS |
| | KDKNIVEIWKLTCGLKGKLSNLFAVNTEDDEAKNFSISFRDGSFDEKVTVVENILGEE |
| | QFDIFMLIKEIHDHCVLANIMKGYEYLSFARVDSYEKHGKDLAILKKLIKENAPGEYC |
| | GMFREMANDNYSAYVGSVNYKNGPVRRGAKCDRDAFFKKVSTLVSKMPESQDRE |
| | YVLQEISKETFLPKQLTASNGVIPNQVHKKEMKKILKNAEGYLPFLLDKDESGYTVSE |
| | RIIKMFEFQIPYYVGPIAEKVGEDSKSKNVWSVRLESGRVYPWNFEQKIDVKESAEKF |
| | ISRMVNKCTYLNGEQVLPKNSLLYERFTLLNELNNLRINGEKISVELKQNIYNDLFKS |
| | GKKVTARKLKEYLKTNGYVEAKEDPVISGIDGDFTNRLANYGKFAAVFGVETLTYD |
| | QEKMAEDIIKWSTLYGDTKSFLKQKITENYGEDKLTKDQLKRILGFKFKDWGRLSKA |
| | LLETRGCSKDDGVIDTVIGRMWNENYNLMELIESRNFTYKEEISERSMAVDKLLGEI |
| | KYEDLDELYISAPVKRMTWQTILALKEICKVMGYGPSKIFVEMARDPNAEKKRTESR |
| | KSKFAYLYRSVKDDTFDWQKAIKETDEGQFRSKKLYLYYTQKGRCMYTGERIELSD |
| | LFKDNLYDIDHIYPRHFVKDDSLDNNMVLVKKEKNAHKSDTFPIEADIRGKMSGFW |
| | HALADGGFITKEKLKRLMRTDGFSDDERVGFINRQLVETRQGTKVITDLFKQTFPDT |
| | DIVYVKAPNVSDFRHEFDLIKCRMINNFHHANDAYLNIVVGNVYDVKFTRNPYNFIK |
| | DYEKGKEKYNMAKMFKYNVSRNGVTAWSIDGEKSIDTVRKVMARNTPLVTRMNY |
| | EAHGALYDQNLVTKRGITKANRDAYLPAKATDDRLAAVEKYGGYRKYTGTYFFLV |
| | EYTEKGKRVRSLEALPLYLKDHYNTKEQLEEYCRERLGLKEPSVRYRRIKMYSKIRV |
| | NGFEVLLTGRSGKQLVISNAQELKISYEYAKYIKKLAEFKEKDVQEEIYEENGITREE |
| | NKKVFLVLKDKFVKEIYSKRVNPIGNRLDEDKFDGLSVKAQAEIILQVVGITQQVNY |
| | GADLSLFDESKTSGVMFLNKMISKQREVVLLNQSVLGIYEERVDLLTI (SEQ ID NO: |
| | 24) |
| 2181613 | MSKLVLGLDIGITSVGYGVIDLDENSFVDYGVRLFKEGTAADNETRRAKRSHRRLLS |
| | RKKTRLADLRKLLKAYQIIDDEQFESLDNVYEIRKLGLTSQLTNEQLASAMLHIAKH |
| | RGSSIDTIDDQKENEKEAEALKDTLSKNAILLSSGKYVCEIQLERMKEDGKVRGHQN |
| | NFKTSDYLNEAKEILNHQQIDENLKTQILEIIARKRAYYEGPGSFNSPTPYGRFIEVDG |
| | QIHEIDLIEKMRGKCSIYPNELRAPKMSITAELFNFLNDLNNLSVKDHKLDIASKKEIL |
| | MAVSQNGGITFKQLAKKIGVNEEDIKGYRIDTKKKPLLTEFKGFKVMRKIFSQCSQIV |
| | SLEDYEMLDDLIEIITKKKGIEERKEAFVQTGYPLSDELIEKLAVVSGVTGYHSLSLKA |
| | MRELNTEMYETTFNQMQLISQMENNSQKQVSFKGKKEIMADETAILSPVTKRAQRE |
| | AFKVINALRKQYGEFDTIVIETTRDKNSEERKKRLRETQKFYEQQNKQVDELLKERG |
| | YDLTKLSGKTKTKVRLYLQQEGKSAYTLKPLDLDSVIRDETYTEIDHIIPISISLDDSFN |
| | NKALITHGENQAKGNMTPYGAYQNGKFADSGCDYAKYNTFVKNAKNIPYRKKQNL |
| | LFEENINKFSVIQEFINRNLVDTSYANRVVLNTLTQYFKDNEIPTKVHTIRGALTDKFR |
| | TQIQLKKDREEDYLHHAIDALIVASVKKMNLLNGYLSKYSFNQLYDETTGEVLEIPN |
| | EKAYFDTKYIQFIVGLKNLYYESNQFYNGLIAREKMQFAPIKLSHKVNTKPNRQVAD |
| | ETIYSTRKVDGQDILIEKIKNIYDPKMTRLTDDIINQNSQKYLMAKHDPQTFELIEKIII |
| | DHFNTYKDSPKHYKANTKKGIITYQLVGENPLTAYKNEFGFVCKYSKKGNGPAIISM |
| | KYESEQLGNHIDISSNYNVSDKKVILKQLSPYRTDFYQCSDGKYRFVTIYYKDVHYQ |
| | KSIGKYIIDKTWYQKEMDKKKIDDKAQFLFSLHRDELLGVVKNDGEKYIYDLSKEND |
| | GDIRYHDGQNVEILKFTATNNDKKGIVEVKPIYTYCKKQLMISPLNFKIISKYATDVL |
| | GNLYEVRQNILKLEFD (SEQ ID NO: 25) |
| 2181731 | MEYNLGLDIGVGSVGWCVTDINGNIIKKNNKNLWGSRLFNEAQTAKQRRTYRGSRR |
| | RLERRKERINMLQSLLLDDMEKQYPNFFPMLRDTSLFIEDRKVNKDKYNLFNDTSFS |
| | DPIYYDKYPTIYHLRKHLVESKEKEDIRLVYLAIHHIIKYRGNFLYEGDLNENFDNTK |
| | LSILVDFLKENDIDENNNEEDIKKIITNVADNKATKKELFKSLFHTQDTITKNILDNVS |
| | KALFGYKFDLNKIFKSDYEIKSISFREEIPDEEDIIEMLGDDAELYVILKDIYNYMVLY |
| | DIKKDFKYVSDAFIEKYNQFKIDLKLLKSIYRKYYASEYSDMFRKKKENNYWHYNK |
| | EILVKSDKKQSKFSEEEFFKTIKKKIENLPDSFNEKENILSRINEGDFLKKLNTTDNAAI |
| | PYQLNKIELEEILKNQSQYYETLSKNNEKIISLLIFKIPYYAGPLSQGDSSKWGWVIRN |
| | SDEKIYPWNYDKVVNVDKTAEEFITRMTNNCTYLVNESVIPKNSLLYSEFCVLNELN |
| | NIRINGYPIAKDFKIKIVKQIFKNKNSVSEKDIRKFYKENGITVTSFTGLSKDDGKFISN |
| | LKSYRDFEKIFTNINDNNKDLCEEIIYWITIFEDKDILKQKLENINLNQQQIKAILSLNY |
| | SGWSRLSKKLINELKSNTGETIMDILSNTSMNFMQIITDEELGFDKQIDAMLPKGSNK |
| | LSYTELIDELPTSPANKRAIWQTLKVVKELKKVMKGSPSNIFIEFARREDEKIRTTDKL |
| | KTLKKIYDEIDKYNRDENVYNELKAHINDKNISEKLYLYFMQHGKCLYSGTPLSINEL |
| | NLYEVDHILPQSYTSNDSIDNKALVIKRENQRKKDSLLLSDTIINKQINWWKNLLDNG |
| | LISSIKYANLTRREIIKTENDGEKFVKRQLVETRQITKSVANILKNNFENTNVLTIRAYL |
| | VTNVRKKYDLYKNRNVNDYHHAYDAFILCQVGNIINNKFKHKEKFVYGDYVKKYI |
| | EEEYEKYLKTKKLSSDGIVIGLIRKYIDATKLKKVLYYKDCYISRMLEENTGEFYNQT |
| | IYSHKFSDNLIPLKKYLPANKYGGFSNLNKAYMAVYSYEEIMKNSIKKRYDLIGIPINI |
| | SYDIRNGKITLYQYLEETLKDKKIQNINIIIERLLIGQQYSDQNGNTFKLCSEQEIRTDK |
| | QLILNELVSKCVSIINKDETKRSQEENGFLKDNYEIVYDYLVEKINNHYTFDTFSSIAN |
| | KLQIKKNDYNSLESNEKEEVLKEIIKLMSTSQGNLKKLGLGERAGRMSGKSFKDETL |
| | LGMTFINKSVTGIYEKRVTINGLENNSNKSSM (SEQ ID NO: 26) |
| 2182400 | MEYYLGLDMGTNSVGWAVTDRQYHILRAKGKDLWGIREFDEACSAEDRRATRTSR |
| | RRWQREKARIGLLQDYFKDEIDKVDPSFYQRLDNSFFKIEDKDSEVQYRNILFNDQN |
| | FTDADYNRQYPTVFHLRCELIKDPSPHDVRLVYLAALNIFKHRGNFLNPSLSVSGTQI |
| | DLTETYSVFRNMLNTYTEIQFPNAFDETLFRSIMCSRRIGRTQRFEALKQFFGVEKAE |
| | KQKLEILRALAGLKSSAAKLFSIETEEKLEFCFSDAGYDEKLSELEAAIGDDDSSILGS |
| | MKQIFDACAWDTIMDGYQYLSEARIASYDKHKKDLKVLKSVFRKYKTPEDYDRFFR |
| | SDADGSYGDYVGSYNSGEKHRRRGSKHTAEDFYSAVKKVLKDCSQDDENVRYILSE |
| | IELGTFMPKQLTASNGIIPFQAHYKELKAILENASGYLQFLNNINPESGLTKKEEILRIF |
| | SFKIPYYIGPLTEQSKKNGGNGWVIRKEAGPVRPWNLDKKVDIAATSEEFIQRMVRR |
| | CTYMAGEKVLPKASLAYERFSLLNELNNIRIDGERLDTSLKQEIYEELFLGGKGVTRK |
| | RLASYLAGRGLIENENQISGIDERIHGQLASYCRFYNIFGDELKKDSCREMVEDIIYLS |
| | TIFGDEKKFLREKLNKKYGDRLDRNTMKRILALKFTDWGKLSRAFLNLQGVQHGAN |
| | ECMTLSEALWRTPYNIMELINSDEFSFKENLSREINNQLGVLQDVTFDDLDEMYFSAP |
| | VKRMVWQTLLVMKELEKVLGAAPSRVFVEFTREHQESKRTVSRKQQFLKLFQGSEF |
| | EQEWKDLIERSEENGTIRSKKMYLYLHQKGIDLYTGQPIALEDLFDSGRYDIDHIYPQ |
| | SLTKDDNLDNNMVLTDKTVNNRKQDNYPLPGEMRRKMQGLWFSLHQSHFINDEKY |
| | KRLTGTEPLTDDQLAGFIARQLVETSQGTKAVADILKCVLPADSKVIYSKAHTVSDFR |
| | NQYGLVKSRAVNDLHHAHDAYLNIVVGNSYYVKFTDSPYRYIKERYSKGRDRYNLP |
| | RLFETDISNGKETAWIGSGKQNGNSENMKPEKGTIVTVRKMLSRPSPLMTRLTFTGH |
| | GGLTNQTIYSAEKSKPQIYLPLKGTDARYDVTKYGGVTSASTAYFILVESVKGKKKIR |
| | TLEAIPIYVASQVEKDSSVLEKYCKDFLKIEKPVIVLRKIKLQSLVKWNGYFLNINGK |
| | YDDSRISVRNATQMLLSQESVKYIKKLEKYSEKRELDTAITEEHNLELYDELTMKHT |
| | DGIFSRRPNPIGDVLTKDRPVFEALSVTDQITALLEIIKTTAIVVGGQADLTLIGEKKGS |
| | GRMKIPKTISSASEMKLYYQSITGLFCEVVDLLKI (SEQ ID NO: 27) |
| 2182512 | MKIGLDIGIASVGWAVVDDDYKVLEAGSNLFESADASKNVERRSFRQLKRLHRRQQ |
| | NRISDFNKLWKKQGLEIPQKVCNTQLSLRVKGLTEKLTNDEFYYVLVNMLKHRGISY |
| | LDDAIEDEGKSSSKYEMGIKANKEQLDEGKFPCQIQMARLDKYGKYRGEIIDGDMIM |
| | SNVYTVSSYEKEIEAIFEKQKQENNCITDNFEKEYISIFNRKRKYYEGPGNELSRTDYG |
| | IYTTKIDENTGRYKTELNLFDKLIGKCTIYPDLRRAAGASYTAQEFNLLNDLNNLIINN |
| | RKLEEEEKHKIVQTILVSNTVNMRKIIEKVIGEKIEVMSGARIDKDEKEIFHKFEQYNK |
| | MRKELNEISVDITSFERDELDKIGEILTLNTDKEAILLAFENEGLNYSEEIKECFVQIRK |
| | KNGSLFSKWQSFSLQIMNELIPEMYIQPKNQMELLTDMGVFKTSADVYKNYKYIPKD |
| | IITQEIYNPVVGRSIRITIDILNALIKKYGYPEKIVIEMPRDRNSEEEKQRISSEQSKNEKE |
| | LAKIIKSVHDEYGIEITKKDFYRHKQLGLKLKLWDEQGRRCPYSGKPIAIEDILNDPY |
| | KFEIDHIIPRSISFDDSRSNKVLVYGVENQEKGNRTPYMYLTNVKREWDWHSYMSYI |
| | LELKANKLIDKSKVDKLLFTEDITKISVLKGFIARNLNDTRYASRVVLNTVQSFMKAK |
| | EADTKVKVIRGSFTHQMRKAMRLDKNRDESFVHHAVDAMLICYSQMGYDTFYKLQ |
| | QGFIDFEDEKVTDKSSWDECMNEEVFERYMYTDKWSKIRENISDAEKRVKYWHKV |
| | DKKANRGLCNQTIRGTREYNGKIEKINKINIYSRDGYKTFVNMINRGKEEQFLMYKN |
| | DRRTFEDILKIYNQYKDAVNAFTEYEKETGDYVRKYAKNHKGCRIENLKYRDGEVG |
| | ACIDISNKYGCKEGSKKVILESLNPYRMDVYYNTNDKKYYFVGLKMSDLKFSGGKY |
| | MLDMNKYNNVLVKEKMLKEGENREDLLRKGYEFRLSFYKNEYIKYDKDGELYIERF |
| | LSRTMPKARNYIETKPIEASKFEKQKLVGLSKTEYVSKVRVDILGNQYECKREKFELY |
| | IDNTF (SEQ ID NO: 28) |
| 2182747 | MGIRIGVDIGIASVGWAVVDDEYQVLESGSNLFDSADASRNVDRRGFRQIKRLHRRR |
| | KNRVHDYNSLWCAYGNKIPDENIVDVLDARIKGLSSELSLLELYCVLRSALLHRGISY |
| | MDDALDDASSGKSDYQKGIQINQEKLGQDLFPCQIQKERLEKYGSYRGTITVTENNE |
| | KVTLSNIFTTSAYVKEVKSILENQMAYHSFIDSDFVDSYVQILMRKRKYYEGPGNEFS |
| | RTDYGIYTTKKDETGQYITEKNIFEKLIGRCSVYPEEIRAAGASYTAQEFNLLNDLNN |
| | LVVNERKLTEEERRKIVDVIKNSNSVNMRKIIAKVIGEDIETMSGARIDKNDKEIFHTF |
| | EQYRAMKKEFAVNGWNIDDFTRDEQDEIAYILTLNTEKEGIMEAFNTAKINLLPEQK |
| | ELLVQFRKKRGSFYSKWQSFSLKIMKEIIPDMYITGKNQMQILTDMGVFKTSKEKFV |
| | NTNKIPADTLLENIYNPVVCRSIRITVNVLNALIKKYGYPDQIVVEMPRDKNSDEEKA |
| | RIKDMQKKNEKELAGIIKKIRAEHGITIKDEDFHHHKNLALKLKLWNEQKGKCIYSG |
| | RPIEIEQLLKDQWAFEIDHIIPKSISFDDSRMNKVLVYEGENRDKGNKTPFMYLSKLK |
| | REWDFEMFKNFVCSMEIPSGKKQKLLFMQDITKVEVLQGFIARNINDTRYASRVVLN |
| | ALQDYFMAKETNTKVRVIRGSFTSQLRKVLQLEKDREESFSHHAVDAMIMCYSQMG |
| | FDVYHKMQMEIVDEETGEIFKVGEFDRVMSNKVYNEMMYQNKLITIRKNIAAAKEK |
| | TKYWHKVDKKANRSLGNQTIRGTRNKNGKVIKINKLDLYDISDVKRFKEKIKKGKE |
| | SSFLMFEKDPKTWEQLMLLMSDYADSPNPLQDYLEETGDYFRKYAKKHNGPKIQCI |
| | KYYDGEVKSCLDISHKYGFSKGSKKVILEGINPFRMDVYCNEKKKEYILVGVKYADL |
| | KYTQGRYVIDEDAYTQILRNEKMIQNDQVYTDVEENGYEFQMSFYKNEIIQYEKDGE |
| | FFTERFSSRTMPNKKNYIETKPVDRPKFGEKKQNLVGLSKTNLIEKIRTDILGNRYKA |
| | NKEKFSLWVDLY (SEQ ID NO: 29) |
| 2182914 | MTICNDYYLGLDIGTSSVGWAVTDPQYNLIRAKGKDLWGVRLFREAETGEQRRYFR |
| | ISRRRRQREIARIGLLKEFFADEIIKVDPSFYQRLSESKYHYEDKEVSSKHSLFSDKLFT |
| | DKEYYKKYPTIFHLREELIENQEQHDIRLVFLTILNIFKHRGHFLNSSLEVNSNNNFIFA |
| | YEELKTLIADRLEIELDSSVEAKEFEKILSAKGFSRTRLAEKAADLLGIKKTKDKQVY |
| | EIIKSLCGLSFSVDIIYGEKFESDGQKTPGISFRESDYEEKIVGIKEVIDEDYSEILDLLKR |
| | LHDQALLSNILQGKDYLSQARVASYEKHKKDLLVLKRAVKEFCPEQYNILFRSNEEG |
| | SYSAYIGSVNSDIKQRRSLKKGKQDDIYSTIKKILVEAPKEHPDINYILNEIEAENFLAK |
| | QLTTSNGVIPNQLHLSELHAILENAKNHFPFLQATDDNEITVAEKIMELFKFQIPYYVG |
| | PLNGYHKGKGGNAWVVRKRQGQVLPWNFDEMIDSKKSSQEFIERLIRKCTYLKDES |
| | VMPKSSLLYEKFMLLNELNNLKIRGVKIDVQLKQKIYCDLFLSGKKVTRKKLTNYLV |
| | SEGIILPNEEGAISGVDGDFKNYLASYGKFISVLGDIVRREDTQNRIERIIFWGTVYGG |
| | DKSYFKENLCEHYSEELSKEQIKRILGLKFRDWGRLSKEFLEMPGVNKETGEMLPLIQ |
| | MLWSYNENLMELMGERFTFKEELDKEVGAITKTLTEMEHSDLDDLYLSSPVKRMV |
| | WQTLLVVKELEKVMECPPKRIFVEMARGSGEKGKRTSSRKEKLLSLYKSCKEDGRD |
| | WASEINTKTDSELRGKKLYLYYIQHGLCMYTGEPIEINQLFDNNLYDIDHIYPRRIVK |
| | DDSIENNLVLVKKQTNAHKSDIYPIEDNIYNRQKNFWKSLLSKDMKDGFITREKYNR |
| | LINRTPLSDEQLAGFISRQIVETRQGTKMLTQILERVFNESDTVFVKAGNISDFRHDFD |
| | LLKVRSVNDFHHAQDAYLSIVVGNVYYIKFTRDPMNFIKNFRGGASNESYNMNKLF |
| | SSNIRRGNETAWVADGSIENVNSIAIVKKHVNKNTPIITKMIYEGHGELFNATLYSSK |
| | KANQKAYVPLKASDNKLVDVTKYGGYSSVSTAYFFLVEHEVKGQKIRTIEALPLIYK |
| | EAIGNDVDNLHNYCVESLGYINPDVRVPKIKMQSLIKRNGFYLNITGKTGDQFIVSNA |
| | VPLILAPSWAKYIKKIEKTIETQFLEREVSKEKNNGLYEELESKHLSCIFKTRPNPIGNI |
| | LKEGKAQFKELDEKSQCLVLAQIMQISQLSNMGANLELIGGSAKSGISLISKKISGVDE |
| | FVLVNQSVTGLFRSEIDLLTI (SEQ ID NO: 30) |
| 2183116 | MAQNYYLGLDMGTSSLGWAVTDRDYHLLRAKGKDLWGVRLFPEAESAAGRRTQR |
| | VSRRRLQREKARIGFVKDVFADAINEVDPGFYQRLDDSKYFPEDKTEMQRFALFSGN |
| | GFTDKEYYDQYKTIFHLRSKLIHSAEPMDVRLVYLAVANIFRHRGHFLNANLTDGQS |
| | AELEEVYARLVDATSNLPSTVNIEKLKEILSSNKVTNSLRNEQLMELLQISKKMPEAE |
| | MLKLVCGLKGMLSKAFTEVEFDEEHQKYSVSFRDGNYEEKDAEMQTLLDEESYGV |
| | VQLLKQMHDWGLLANIMTGEQYLSDARVKSYEKHAYDLKILKDLYKEYGNGQYN |
| | KMFRVMEDHNYSAYVGSVNSDKEKGKKRRGAKKDRDGFYKKLKAEMQTMQKKA |
| | PDDVRIRYILDEIDKEALLPKQLTSANGVIPYQVHLKELKTILSNAEQYLPFLRDKDET |
| | GLTKSEKIQQLFAFQIPYYIGPLYNDGNASHNAWVCRKEAGKVFPWNFDQKIDVKAS |
| | SEEFIRRLVRKCTYLTSENVLPKSSLLYQKFMVLNELNNLKINGVSIDTELKQEMYRD |
| | LFCREKKITSKKIKEYLITRGVCPKKTGAEISGIDGDFRSSLSSYLLFCEILKVSVLTYE |
| | QEKMAEQIIFWATVYGDTKSFLKEKIEENYGDKLTAAQIKRICGIKFKDWGRLSKAM |
| | LELEGADRETGEVLTIISRMWNENVNLMECLSDKYTYLDTIQDEARAISKSFDEIHYD |
| | DLEELYISAPVKRMVWQTILVVKELVKVMGGPPAKIFVEMARDVDGKNDKIRKDSR |
| | QKKFADLYKKCKDDGRDWSKEIAETPESKFRSKKLYLYYTQKGRCMYTGEPIDLGN |
| | LFNDNLYDIDHIYPRHFVKDDSIEKNLVLVKKQINSHKSDNYPLETEIRKSQFGWWK |
| | HLCDNGFITKEKFDRLIRSTQFSDEELAAFISRQIVETRQGTKMVTELFENSFPEAEVIY |
| | SKAGVVSDFRHKFNLLKCREINNFHHANDAYLNIVVGNTYNVKFTKHPANFIKDYQ |
| | RDPEHFKYHMDKLFDYPVSRGSEFAWKTKGGESIKTVRKVMSRYTPLVTRMNYEE |
| | HGQLWDQTIYSADDAAKAKGVGYVAVNTSDPKMSDVAKYGGYKKYTGAYFFLVE |
| | HTKKEKKVRSLEAMPLYLKDSLDSKEKIEAYCREELGYQEPSVRIEKIKMYSLIKVDG |
| | FLLYLTGRTGNQLNVINAVEMKLPNEWNTYVKRLFSSSTVIDSNNDISTSGDLISKEK |
| | NISLYSILAEKHCAGIYSKRPNPIGTKLKDWKDKFEELPVDKQVYVLKQIMQLSSNSN |
| | QGADLQCLGGAAKTGVSLLNKVISGYKEFVLVSYSPTGIYKSENELLTI (SEQ ID NO: |
| | 31) |
| 2183137 | MEYYLGLDMGTNSVGWAVTDPQYRLMRAKGKDLWGARLFQDAKTSATRRTFRTN |
| | RRRKQRERARIGFLQECFSDEINAKDAGFFQRLEDSKFFEEDKTEKQPYALFADTGYT |
| | DVEYFKDYPTIFHLRKELIQSSEEHDVRLVYLAVLNIFKHRGHFLNQNLSDSGLPDLE |
| | RIYTELVQNYAESLSLDFSEMSDLDYSAIKDLLSTKKLSNSRRAEELTKQFGINKKSA |
| | SGEMIKAMCGLSATVVKMFPNETIEEESDVYKMKFSFRDSDLEGKLAQLESALSDEA |
| | YECLILIKQIHDWGLLASIMTGNGKNYTYLSEARVDQYNKHHRDLILLKKVVRKYSP |
| | DKYNEVFRIMKDNNYSAYIGHVNSRIEKTRRGGTKRQEDFYKTINSLLPNNTEDPDV |
| | RQIKDEMERGTFLPKLLTNSNGVIPMQLNKIELSAILHNAENYLSFLKKKDENGLTVS |
| | EKIVKMFSFQIPYYVGPLLNTENNNAWVVRKEGGKVYPWNFEQKIDVKKSSEEFIAR |
| | MVKHCTYLGEETVLPKASLLYEKFMVLNELNNLRINGEKIAPELKQELYKALFLKGK |
| | KVKAKQVVEYLQSRGLVEKEREVELTGIDGDFVNTLSSNAKFCAIFGVDQLSAEQEK |
| | MAEQIIYWSTIYNDSKKFLQEKIQESYGDILSTSQIKRITGIRFREWGRLSREMVELEG |
| | ADKENGEIKTIIHRMWDENVNLMELLSNRFTYTQAIENRTRLIDKTLNEIEYEDLDGM |
| | YLSAPVRRMIWQTILIMKELYFVLKKAPAKIFVEMAREGNAEKVRKQSRKKKFAEL |
| | YKNIKDEEINWKELIDSTDEGKFSSKKFYLYLTQCGRCMYTGERIELEDLFKDNLYDI |
| | DHIYPRHFVKDDSIENNLVLVKKEKNSHKSDVFPLERSIRSQQASFWKTLCDHKLITK |
| | EKYNRLMRTESFSEGEMAQFINRQLVETRQGTKMIAKLMQRTFPDSKIVYAKAENVS |
| | LFRQKYNLIKCRRVNDFHHAQDAYLNIVVGNTYSVKFTDDPRRFIQEFKKDPNHNK |
| | YHMYKLFDYDVVRGVEKAWIAENGESIGIVRNMMAKNTPLVTQYSYKAHGGFADQ |
| | TVYSAKKAGAAKDGAYMPLKSKDPRLCDVAKYGGVTKISGSYFFLVEYTEKKKRIR |
| | SIEAVPIYLAEELNSTGALKEYAEKTLGYTDPDVRIPCIKINSLLEVNGFFATLTGRTG |
| | DRLILPSATQLKMDKSSVDYIRLLENNAGEKDDSRLQYKKVSREANEILYGLFIEKHL |
| | YGIYSRKPNAIGQKLKDKESVFKNLTIGEQVEVLLQILQISCRVNTGADLKLLKESSST |
| | GITLISKHISNQQSIFMINQSVTGLYQKKIDLLKI (SEQ ID NO: 32) |
| 2183156 | MEYYLGLDMGTSSVGWAVTDSSYHLLRAKGKDLWGVRLFNEANTAADRRVFRTN |
| | RRRLQREKARVGFLRECFADSINQIDPNFYQRLDDSKYFLEDKTLNQPFALFSDKEFT |
| | DKDYYQKYPTIFHLRKELIESSEPHDVRLVFLAILNIYKHRGHFLNQNLSDDGIGDLN |
| | ELYCLLSQDDYFCFPDLKETEEIKSILSSKSISNLQRSEEIVKFLNLDKKSKEAEAVKMI |
| | CGLTGTISKIFAISEDDEEGQKLKISFRDGTLDEKLNQAETTLSEEDYERLLVFKQIHD |
| | FGLLSSIMPSSGSKYLSYARVESYEKHHRDLLLLKKVYKKYMPDKFDEMFRIMRDN |
| | NYSAYSGSVNSKIQKERRGGKAKTEDFYKGIITDIAKMPKDEDTEYITNEINKETFLP |
| | KQLTNSNGVIPMQVHKMELAAILKNAENYLDFLKVKDETGIAVSQKILDVFSFQIPY |
| | YIGPLVNNGDNNAWVVRKEEGKVYPWNFEQKIDVKASSEEFIKRMVNHCTYLTGET |
| | VLPKQSLIYQRFMVLNELNNLKINGEDITVELKQDIYNDLFKKGNKVKGSTIIKYLKS |
| | RNLIDAKDTPEITGIDGDFVNSLSSLKKFRDVYGVDELSGEQQKMTEQIIYWSTVYGD |
| | SRKFLNEKITENFGDILKKEQIKRITSLKFKDWGRLSRQMLTLEGASKGTGEIKSLIGR |
| | MWDENLNLMELLSDRFTYLEEINKRVKAINKSLSEITYEDLDELYISAPVRRMTWQTI |
| | LIIKELYSVLGEAPRKIFVEMARDTDAKKERKDSRKKKFIDLYKSCKDDGVNWSKQI |
| | KDTPDERFRSKKLYLYYTQKGRCMYTGEIIELQNLFNDNLYDIDHIYPQSVVKDDSL |
| | ENNCVLVKKTENAGKSNTFPIAESIRHKQASFWKTLLDGEFITKEKYNRLMRREAFS |
| | DDELAQFVNRQIVETRQGTKFIAELFKEVFPDSDTVYVKAGNVSEFRKRFDLLKCRSI |
| | NDFHHANDAYLNIVVGNVYDVKFTKHPINFIKEYRKDPKSHPYHMNKIFDYSVRRG |
| | DQIAWIAENGESIAVVKKMMQKNTPLVTQMNYEVHGGFAEQTIYSADLVNKAGGE |
| | GYIPVKCNDKVANINKYGGYSSVKGTYYFLVEFTKGKKRIRKLEFLPLYLKNSLNDI |
| | KALEEYCKDKLHYEKPSIRLCRIKMNSLVKRNGYFVTLNGRTGMQIYASNAVQLIM |
| | DKKHQDYVHLLDKYSGINDSEYLKRNNISKESNEEIYQYLVEKLNNKIYDNRPASILS |
| | KLKSKYNVFKELELSEQNYVLKQIVLYFSRANISIDLKLLNEKSSPMKISNTVSNEKEF |
| | ILINQSVTGLFKNEINLLTI (SEQ ID NO: 33) |
| 2183168 | MNYNVGLDIGVASVGSAVVTEDYEIVESTSNIFPEADAAENLTRRSMRQNRRLTRRR |
| | KTRITDFVKLWKRMGFDVPESFDCNVLELRVRGLTEELSMDELFLVLQNDLKHRGIS |
| | YLEDATDVSDSSQYAKGLKKNHEEMEDKYPCEIQLERLSKYGFYRGQHTIKESNEEV |
| | VLSNVFTVSAYREEIEQLFKTQRKYNEKITETFEEKYLDIFRRKREYYVGPGNEYSRT |
| | DYGKYTTRVNPVTGEYITEENIFEKLVGKCSVYTEERRASAASYTAQEFNVLNDLNN |
| | LVVNGRKLEKDEKIEIIRLMKTEKTVSVPRFLKKVMGETVESIEGARIDKNDKQIFHS |
| | FEIYNKQRKAFDELGMNIDDIFSVDELDLIGDILTLNTDKDSILSALNRNGFLLDEQIS |
| | DCLISIRKKNTNLFNKWQSFSIKIMRELIPAMYEESKNQMQLLSDIGVFKSNTDKYKG |
| | LKYIPDKELLEEIYNPIVRRSVHTSIKVLNAYIKKYGYPQKIVIEMPRDKNSDEEKKFI |
| | KKIQDENEKELDNIKKKCKSEYGFEIKDEHFRNHKKLALKLKLWNEQKGKCLYSGR |
| | DIDIKELVENSSSFEVDHIIPLSISLDDSRNNKVLVYAIENQRKGNNTPYKYLKGIDRD |
| | WDYEKYREYVLSLGLTNSKRKKIQNLLFEQDITKLEVVRGFINRNLNDTAYASKLVL |
| | NTLQSFFKANDTNTKVYVIRGAFTHQMRMNLKLDKDREINSHHAVDAMLIAFSKMG |
| | YDAYHKLQENVIDFETGEILDGHDENDIVDDATYKKMLYEDKWFEIRTRISKAEKEV |
| | KYNYYVDKKINRGLCNQTIRGSREYDGKIYKINKLNIYTQDGIKTFKKKIEGGKEDQF |
| | LMKKNDPKTFEDLMSIYNQYKDAANPFVEYEKETGEYPRKYSKKHNGPRITNMKYT |
| | DGEVGSCIDISHKYGYEKGAKKVFLESLVPYRMDVYFNKNDNLYYFVGLKHSDFKY |
| | IKGKYMLDDDAYNRVLQEEKLIDSTQTHKDLEARGFEYRLSFYKNDLISYEKNGEIY |
| | ERRFISRTMPQKRNYIEIKSVDGKELDKRTDGLSKAMNVTKIQTDILGNKFYVKREKF |
| | LK (SEQ ID NO: 34) |
| 2183254 | MGLRFGFDIGIASVGVAVVDDNYDVKEAISYIFPAAEASENVSRRSFRQMKRLHRRK |
| | ANRLNDFNKLWIKYGYKIPDIKINEVIELKVRALSQEISMDELYAVLHHSLKMRGISY |
| | LEDAIDENGAKGDYQRGIAINQKELEEKEYPCEIQYERLRKYGSYRGENTVEIDGEK |
| | VTLSNVFTTGAYKKEIEVILKKQSEFHGEISESFIKDFMDIFSRKREYFVGPGNELSRT |
| | DYGKYTTKKDAEGNYITEDNIFEKLIGKCSINPDETRAAAASYSAQEFNLLNDLNNLT |
| | VNERKLSEEEKRKIIDIVKNSNSVNMEKIIKKVIGEDIYKWTGFRVDKNDKPEFHTFK |
| | QYNLIRKALEEIGVDIQDFDRDTLDNIAYVLTINTDKEAILKGFDRENIELSDEVKEVL |
| | ISVRKKNSSYFNKWHSFGLTIINELIEPLYKEPKNQMELLTNMGYFKPDVERYKDYNS |
| | VPVNELLDNIYNPVVTRAVRITIKALNALIKKYGYPDEVVIEMPRDKNSDEEKKRIDS |
| | NNKANAKELDEIVKKIKLEYGIEISESTFYKHKGLVMKLKLWNEQGGKCLYSGKTID |
| | VKDLINNFDMFQIDHVIPKSISFDDSRNNKVLVYGTENQNKGNMTPFMYLSTVNRD |
| | WNYDKYKEYIKSLGYSRNKRKNLFFEEDITKMEVLQGFINRNLNDTRYASKVVLNT |
| | LQSFFRAKDVDTKIKVIRGSFTHMMRVNLRLDKDRDESYSHHAVDAMLMCFAQMG |
| | YEAYRKLNHDIVDYESGEILDEKELIKSFDDNKFDDLLYQDKWMNIKNGIKKAEQEV |
| | KFWFKKDTKVNRGLCNQTIYGTREVDGKTIKVNYLNIYTKDGYKRLLNLIKKGKQE |
| | DFLMYRNDPKTWENLMTILEDYKDSANAFVEYEKETGDCLRKYSKKHNGPRIEKIK |
| | YLDNEVNSCIDISHKYGFKKNSKKVILESLNPYRTDVYYDLEKKIYYLIGIKYSDIIMQ |
| | HGEYYIDEKKYNELLIKEGAKLDRINTEIVFKFSLYKNDIIKYEKAGEIYLERFSARIM |
| | PNVKGYIETKPVNKSKFEKRNLTGVNKSLFIEKINLDDLGNQYKSVGEKFSLKVDM |
| | (SEQ ID NO: 35) |
| 2183262 | MEQKYYLGLDMGTSSLGWAVTDQNYHLLRAKGKDLWGVRLFQEANVSASRRTNR |
| | VSRRRLQREKARIGFVRAVFADAINEVDPGFYQRLEDSKYYPEDKAEHQKYALFTGE |
| | RFTDKEYYEKYPTIFHLRSDLLKASEPRDVRLVYLAVLNIFKHRGHFLNANLSDEKT |
| | NGFEHIYAQIAERIEKFPSNPKFEYLKEILSSNKGTKSQKAEQMIAALHFNKKDPEAE |
| | MAKMVCGLKAVLPKAFPDCYFDEEHTKYSVSFRDGNYEEKETEMQEFLDEETFEIV |
| | QLLKQMHDWGLLANIMNGEEYLSDARVASYEKHASDLKILKDLYKQYGCGSYNK |
| | MFRVMEDHNYSAYVGSVNSDKERGKERRGAKGNRDDFYKRIKNDLKKMLEKASD |
| | DARISYVLDEIEKETFLPKQLTFSNGVIPYQVHFRELKQILENAETYLPFLKEVDETGL |
| | SNTEKILRLFSFQIPYYIGPLYNDGNPAHNAWVVRKSRGKVFPWNFEEKVDVKASSE |
| | AFIQKMVRDCTYLQGETGLPKESLLYEKYMVLNELNNLKVNGVPIDVELKQNIYLD |
| | LFTHHKKVTEKKIRDYLITNGAVAKNIPTELSGIDGDFKSSLRSRIIFQDIFETETLTFEQ |
| | EQMAEKIVFWSTIYGDSKPFLKERIEEHFGGKLSEKQIKRICGLRFRDWGRLSKAFLQ |
| | LEGADIETGEIKTIIERMWDENLNLMECLSGRFTYIQEIREQGASINKSFHEFRYEDLE |
| | DLYVSAPVRRMIWQTILVVREITKVMGCPPDKIFVEMARDPGDKNEKVRKDSRKKK |
| | LFDLYKNCKGERDWCKEIDSIEEARFRSKKLYLYYTQMGRCMYTGEPIDLEDLFNDN |
| | LYDIDHIYPRHFVKDDSIEKNLVLVKKQVNSHKSDTFPIEKEIRVAQSNWWRQLCDL |
| | QLITKEKYERLMRNTEFSEAEMAAFISRQIVETRQGTKVITTLFENSFPETDVIYTKAS |
| | TVSDFRHKYGLLKCREVNNLHHANDAYLNIVVGNVYNTKFTNNPINFIKEYQRDPE |
| | KYKYHMDRLFDFTVVRGDQIAWVAKGGESIRTVRTTMAKNTPLVTRMNYEAHGQL |
| | WDVQICSAKDAEKAKGVGYIPIKTTDRKLHDVMRYGGYRKYTGGYFFLVEHVVKG |
| | NTIRTIETMPIYLKRNLDTKEKMETYCRDVLNLEEPHIRVERIKMYSLIKVNGFPMYL |
| | TGRTGNRLVVVNAAELKLSKEWMTYIKRIFSSADEGEQAEGGLNYITRDKNVELYDI |
| | LMVKHCKGIYSKRPNPVGAKLSEWRITFQKLSVDRQIYVLKQILMLSSNTNQGSDLH |
| | DLGGASKTGVSLLSKKVNDYQEFKLISQSPTGIFETERDLLTV (SEQ ID NO: 36) |
| 2183288 | MARKYYLGLDMGTSSLGWAVTDPEYHLIRAKGKDLWGVRLFQEAESAAGRRTQRV |
| | SRRRLQREKARIGLVKEIFADAIDKVDPGFYQRLDDSKYFPEDKTEEQRFALFAGDSY |
| | SDKDYYKEYPTVFHLRSDLIHSADPKDVRLVYLAILNIFKHRGHFLNANLSDGDADG |
| | LEEIYAQLVERTVNLPATYDAGKMKDILSSRKVGNSRRSEQLMELLQINKKMPEAE |
| | MVKLICGLKGVLPKVFPDIEFDEENKKFSVSFREGNYEEKDAEIQSMLDEDSYETVQL |
| | LKQMHDWGLLAHIMGGEQYLSDARIQSYNKHADDLRLLKELYREYGKGQYNKMF |
| | RIMADNNYSAYVGSVNSDKEKGKVRRGAKKNRDEFYSRIKKDLKAFQEKVPQDERI |
| | QYVLDEIDKESFLPKQLTSANGVIPNQVHLKELRKILSNAEGYLPFLRNTDESGLTNS |
| | EKIVALFSFQIPYYIGPLYNDGAHNAWVVRREAGRVLPWNFNKKIDVKASSEEFIQK |
| | MVRHCTYIQSEYVLPKNSLLYEKFMVLNELNNLKINGNPIGVELKQSIYNDLFRREK |
| | KVTDKKIREYLIVRGLASKKDEIEITGIDGDFKNSLSSYLRFREIFSTDVLSYEQEKMA |
| | EKIIFWATVYGDSKSFLKERIKEKFGDQITDKQIKRICGIKFRDWGRLSKAVLELEGA |
| | DRETGEVLTLIGRMWNENVNLMECLSGHYTYIDELQEKGREINKSFEEIEYEDLEDL |
| | YISAPVRRMVWQTILVVREIVKVMKCPPAKIFIEMARDAEGKNDKTRKDSRKKKFEE |
| | LYKGCKEDGREWSKEISDTPDSRFRSKKLYLYYTQKGRCMYTGEPIELGDLFNDNLY |
| | DIDHIYPRHFVKDDSIEKNLVLVKKQLNSHKSDTFPIEAEIRRVRFAWWKNLCDQGFI |
| | TKEKFERLVRSTQFSEEELASFISRQIVETRQGTKTVTDIFENSFPDSEIIYSKAGVVSD |
| | FRHRFDLIKCREVNNFHHANDAYLNIVVGNAYNVKFTKNPINFVKQYKRDPEHYKY |
| | HMDKLFDYPVIRGDEVAWKTKGWESIRTVRSVMARYTPLVTRMNYEEHGQLWDIN |
| | ICSADDATKAKGVGYVPVSSTDGRMHDVTRYGGYKKYTGAYFFLVEHTVKKQRIR |
| | TLEAMPLYLKDKLNSKEKMEQYCKDTLGYQDPSVRLKKIKMYSLIKVNGFLLYLTG |
| | RTNKQLTVTSAVETKLQAEMNEYVSRLIKCTEDPKNVSGAVEKWNELVNKNRSEGK |
| | NNRNTRSLINKENNKTLYDALKEKHTNGIYKNRPNSIGDKLAKYEEEFENITIAEQLL |
| | VLKQILQLTSGSKKQADLSLIGGSPNEGVPTLNKKINGYNQFMLVSMSPSGIYEYETD |
| | MLTI (SEQ ID NO: 37) |
| 2183304 | MEKRDYYLGLDLGTDSVGWAVTDTNYNLLRAKGKDLWGIREFNEALVAADRRSH |
| | RVNRRRRQRELVRIGLLKGYFSDALQSVDPLFLIRLQESMFHKEDKDRALSGNVNGI |
| | FNDKDYTDKDYYKEYPTIFHLRKELIDNKSPHDVRLVFLAILNLFKHRGHFLNATLSS |
| | EGEIRPFISVYKELLVNLQENFEINLPESINEDEILHVLGDRSLSRTRKKEQISNALSIDK |
| | KEKSKDAIIKLFCGLAVKASALIGDSFDSEEDINICFSDAGYDEKSEEIYSKVGEEWSN |
| | VIELCKEIYDIGSLSEILRGNNYLSFARVEDYNKHKKDLEILKKLFHQTSEEAFDHMFR |
| | SNNPGTYSAYVNSTNSNKKTRRNVKERKRDDLYKTIKSYAKGWNPEDPDVKYVLA |
| | EIESENFLPKQLTFANGVIPNQVHLKELKAILRNAEGYLPFLLEKDEYGNTVSNRIEQL |
| | FSFQIPYYIGPTSEKSKEMNGNGWVVRKEAGPVLPWNIDEKIDIKATSEAFIEKMVKK |
| | CSYISDEKAMPKNSLLYEKYRVLNEINNIRINDHRITTELKQDIFNDLFLSGKRVTRKK |
| | LESYLIGRGAIENAAQLSGIDININNYLSSYGKFKDIFGESIKEDSTKNMAEDIIYWCTI |
| | YGDSKKFLKERISEKYKDKLSEKQIKSIMGIKFSDWGRLSKTFLELPGTDKETGEKITL |
| | IRAMWDNNLNMMELINDTEHFTFKESLEDMVISGTKVLSEFSHEDLNDYYFSAPVKR |
| | MIWQTLSIIKELESVLGAPPKRIFIEMTRKPDDKKERKESRGKQLIDLYKNIKAEDKA |
| | WKNEMISKIESADSDGSLRSKKLYLYYTQMGRDMYTGEVIDLDELFNNNLYDIDHIY |
| | PQQLVKDDNLSNNLVLVKKQKNSEKQNVYPLDEKIRKSPKVRDLWNMLHLNNLIN |
| | DEKYKRLTRNTGFDDDELAGFIARQIVETGQATKGVADLLKTLLPEPETKVVYAKAS |
| | AVSEFRKDNGFLKSRLINDNHHAQDAYLNIVVGNAYYVKFTQNPINYIKKEYKDKK |
| | DSYHLGKMFKNTITRGKETAWIAAGEGEEGTIKTVKKVMSRNTPLLTRLSFVGHGQI |
| | SEATLYGADTTKEKGYLPLKEKDDRFLNMAKYGGFSGISVAYFFLVEHISKKKPVRT |
| | LETVPIYIADRIESDPSALKRYCIEKLKLVDPNIRLRKIKLHSLVEKDGYFGHITGVTGN |
| | RIYFRNAVSLILPIEWNNYIKTIENSIEKKSLNDIITANKNISLYDLLTNKLSNSIFAKRP |
| | NPIGDNLQVSRDRFITLNLEEQITLLEQILHLTAIGMAKADLTSLGYSKLSGISLLNKEI |
| | TKSNSFTLVYQSITGLYEKRIDLLTV (SEQ ID NO: 38) |
| 2183312 | MGKIIGLDIGIASVGWAVLDKDSYKIVEAGSNIFPAAEASKNQERREFRGLRRMTRRR |
| | RTRLDDFKKLWKNYYKNEYSVTPNNVTELKVKGLSEQLSELELYTVLYSYLKHRGI |
| | SYLEDAVDEGKTGSDYLKGLQTNQKEMNTKFPSQIQYERLEKYGCFRGENKVSDDE |
| | VLSNVFTTSAYKKEIEELISVQDNLGNELDKDFIKKYFEIFSRKRKYYEGPGNELSRTD |
| | YGIYTTKIDENGNYITDKNLFEKLIGKCSVYPEEMRASAASYTAQEFNALNDLNNLIV |
| | NGRKLTKEEKISIIECYKTEKTINVRKIIKNVIGENIDQLSGYRMDKNDKELYHTFEVY |
| | NKLRKAFEEAGISFDFSREDFDEIGRILTLNIEREGIEDAIRISNLVLSSDAVDVLISVRK |
| | KNASLFSKWHSFSLKIMNELIPEMYEQPKNQMELLSDMKSIKSKMDEFKGLEDIPVE |
| | TIIEDIYNPVVKRSIRITIKIINALKKKYSDIEKIVIEMPRDKNSDEEKERIKKFQKDQEN |
| | ELPNIIKRVKDEYGITLTPELIARKKGLAFKLKLWNEQNGRCPYSGRMIDIESLINDGD |
| | LFEVDHIIPLSISFDDSRSNKTLVYRVENQEKKNNTPFMYLSRLNRQWNYDKFAAFIK |
| | ELYGSSGKDRKKFRKKMNNFLFTEDINKIDVLKGFINRNINDTRYASRVVLNSLQGY |
| | FRANDNNVKVSVINGSFTHQMRVNLSLGKDRDESFAHHAVDAMLIALSQMGYDAF |
| | RARQKDIVDFETGEIINGSAWENQDEMYKTVLYQEKWKNIKDNIIEAEPNIKYWHKV |
| | DKKCNRGLCNQTIYSTRKCDDKIWKVDKLNIRDKDDVKKLIYMIQNGKEDKILMAQ |
| | NDPKTFANLKYILDEYSDCENPFVSYEKETGDLVRKYSKKHNGPKITYLKYMVEEV |
| | NSCIDISHKYGHEKGSKKVFLAQLVPFRTDVYYNNNSGKYYLIGIKQSDIKCKGNQYI |
| | IDEEKYTDNLIQNGLLKEGQKIEDLEKLGYLFKFSIFKNDTIEFEKKGELIKCRFLSQNS |
| | ASKNRIEVKPIGKKDYESRNLPTLSTTTVINKYVKDILGNEKQVSGEKFEIIVDSK |
| | (SEQ ID NO: 39) |
| 2183526 | MKYYLGLDMGTDSVGWAVTDEHYNIVRAKGKDLWGIREFEEAKTAADRRIQRTSR |
| | RRLEREKARIGLLKDYFKDEIDKVDPSFYARLDNSFLKIEDKAEEVQYKNILFNDEDF |
| | TDRDYYKKYPTIFHLISELIHNTTPHDVRLVYLACLNIFKHRGNFLNPALSKDNTRIDI |
| | SDKYTELRQALSEQTNIILSEEFNENSFKAIMCDSSKSRTLKYEGLKDLFSIEKSDKQK |
| | QEILKAIAGLKVNAKNIFDLDTEEKIEFSFYEGNFDDKAAELEKEMGEDFFNIIALMK |
| | EIYDACEWDTVMAGYEYLSDSRVASYEKHKKDLVILKRAFRNNKSKDDYERFFRTD |
| | EDGTYGDYTGSNNSREKHRRRGSKHRQEDFYDSVKKEFKGLEKNGDPDIDYILTEIS |
| | NGTFMPKQLTPSNGVIPFQAHYKELNAILNNASNYLGFLNDIDSESGQSVKADILQIFE |
| | FKLPYYIGPVTTKSAENGGNGWVIRRENGKVLPWNIEQKIDTKATSEAFIQRMVRKC |
| | TFLSDEKVLPKSSLLYEKFSVLNAINAISIKGERISTNIKQDLYHDKFENGKKVTKTQI |
| | VRYLNGRGLDVTENDITGIDDIITSQLSTYHRFYQIFGDDMKKDSYKNMAEDIIYLSTI |
| | FGDEKKFLKDKIKEKYGDKLSDEQIKKVLSIKCTDWGKLSKEFLELKGCRKNTGEEIS |
| | LIDAMWETSLNLMELINSDEFNFNEVLNERQTKAFSSLKELKPEDLDDMYFSAPVKR |
| | MVWQTLLIIKELEQVIGSAPERVFVEVTRSHEESKRTVSREKLLLDLYKNEDKYWKD |
| | IISEASVNGRIRSKRMYLYLKQQGKDLYTGKEIPLDEVFNKGKYDIDHIYPQSLTKDD |
| | SLDNNMVLTDKSVNNHKQNIYPLEKSIRDNCYALWTALHAKGFINDEKYKRLTGNQ |
| | PLTDGQLEGFIARQLVETSQGTKAICDILTQALPATKVIYSKAKVVSDFRNQYHIYKS |
| | RIANDLHHAHDAYLNVVVGNSYYVKFTDSPHRFIKERFRCDGGKYNYNLATFFESD |
| | VSNGKETAWIGSGKEYNNDNEKGTILTVRKMLAKTSPMMTRLNFVNKGSIAKEQLIS |
| | ADKCKKDSYLQIKVNDARLDVTKYGGYQKVSAAYFFLVESAGKKGEKIRTLETVPV |
| | YLADKIEKNPALLEQYCKDILKLNNIRISVRKIKIQSLIKRNGYFLHISGKSSNQILLRN |
| | ATNIFYNQEKINYIKKIEKYVDKQEVDEEINTEKNIELYDYLTAKHNGKILLHRPNPIG |
| | EKLTKRKDKFAELSLAEQILCLYEILTSTAIVAGGAANLKQLGESPSTGVMTMNKNIS |
| | KEKELLLINQSVTGIYTNTIDLLKI (SEQ ID NO: 40) |
| 2183548 | MEEKEYYLGLDMGTNSVGWAVTDPQYIILRAKGKDMWGIREFESAETAETRRVHRT |
| | SRRRRQREVVRMGLVKDYFSDAINNVDGNFYARLENSKYHLEDKDETVRNKNGIFA |
| | DEHYTDKEYYAEYPTIYHLIMELINNPAPHDVRLVYLAVANLFKRRGHFLNAGLSED |
| | GGNQSMQTAYDEFNTTCNETLEFSFPGIDATQIEEVLKNRNYSKTVKSEKLAELLGV |
| | EKKEKQRIEILRAIAGRKVNAKVIFPNVETEDKVEFSFSDAAFDDNATELMTLLGDEC |
| | YSLVLCMREMYDIGTLAEIMHGYQYLSESRMADYQKHHEDLVLLKSVIKSECTQKQ |
| | YDELFRLDGEGSYSAYVNSILAKNHKQRRSMKGRTRDDFYKKVREFIGKSSNPDAIK |
| | ICTEIENETFMPKMLTSANGIIPNQVHYKELKAILSNAEKYLPFLLEKDESGLTTSDRIQ |
| | RLYKFRIPYYVGPVSNRDGKGNGWAVRKEEGMVLPWNLEEKIDLSKTKEAFIENLIR |
| | RCTYISDEKVLPKNSLIYEKFTVLNEINNIRINDERISVELKQDIYNDLFKKGKKVTRK |
| | TLAKYLINRGAIKAENQISGIDIAINNSLSSYGKFKAVFGESMDTDSTKKMVEDIIKTS |
| | TIYGDDKRELKNILRDKYGEILSEDQIKRIAGFRCKDWGRLSRAFLELAGTNCGTGEV |
| | TSIIQALWESQLNLMELLHSEDYTFGESLNEKLVKNEKSLSEITAEDLDDYYFSAPVK |
| | RMAWQTILLIKEISKIKGCEPSKIFIEMTRTDEEKGDKGRKNSRAKELTALYKNIKKE |
| | GKAWIELISKEDASGRLKSKKMYLYIKQMGRDMYTGEQIDLDRLFDDNLYDIDHIYP |
| | RHFVKDNNIENNLVLVNKAKNSRKSDNYPLDSDIVRNPKVRELWEMLHTNHLMSDE |
| | KYHRLTSRKAFTDEDMAGFIARQLVETGQATKGVADLLKDLTNSEIIYSKAGNVSDF |
| | RHTYDIPKSRIVNEFHHAHDAYLNIVVGNAYYTKFTSNPLNYIRKEYKKNTKENNYH |
| | LNKIFASTISRNGYVAWFAGSDKNPGTIQTVKKMLSRNTPLMTRMTFVVHGSIAEET |
| | LYGKNTAKEDSYLPLKGSDPRMSVEKYGGFKKASGAYFFLVEHELKGKKVRTIETV |
| | PVYLMKKLSDKRELQKYCEEKLGLINPSIRLKQIKMQSLIKLNGYFVNVTGRTNKQL |
| | TLRNAVNLKLDKEDIYIIHLVEKCIGEERDDRLSEESILSLYQTLTEKQVSGIYRNRPN |
| | SVYEKLNEGMIRFKKLERKQQIKVLANMLNLTMVGKAQADLKDIGGSANSGVMLM |
| | AKKINNYNEVKLINQSVTGLYQTEVDLLTV (SEQ ID NO: 41) |
| 2183594 | MEEKEYYLGLDMGTNSVGWAVTDPQYRILRAKGKDMWGIREFESAETAEKRRGHR |
| | TSRRRRQREVVRMGLVKDYFSDAINIVDGNFYARLENSKYHLEDKDEAVRNKNGIF |
| | ADEHYTDKEYYAEYPTIYHLIMELINNPAPHDVRLVYLAVANLFKRRGHFLNAGLSE |
| | DGGNQSMQTAYVEFNTTCNETLGFSFPGIDATQIEEVLKNRNYSKTVKSEKLAELLG |
| | VEKKEKQRIEILRAIAGRKVNAKVIFPNVETEDKVEFSFSDAAFDDNATELMTLLGDE |
| | CYSLVLCMREIYDIGTLAEIMHGYQYLSESRMADYQKHHEDLVLLKSVIKSECTQKQ |
| | YDELFRLDGEGSYSAYVNSTLAKNHKQRRSMKGRTRDDFYKKVKGLIGKSSNPDAI |
| | KICTEIENETFMPKMLTSANGIIPNQVHYKELKAILSNAEKYLPFLLEKDESGLTTSDRI |
| | QRLYKFRIPYYVGPVSNRDGKGNGWAVRKEEGMVLPWNLEEKIDLSKTKEAFIENLI |
| | RRCTYISDEKVLPKNSLIYEKFTVLNEINNIRINDERISVDLKQDIYNELFKKGKKVTR |
| | KTLAKYLINRGVIEAENQISGIDIAINNSLSSYGKFKAVFGESMDTDTTKKMVEDIIKT |
| | STIYGDDKRELKNILRDKYGEILSEDQIKRIAGFRFKDWGRLSRAFLELAGADRRTGE |
| | VTSIIQALWESQLNLMELLHSEDYTFGESLNEKLVKNEKSLSEITAEDLDDYYFSAPV |
| | KRMAWQTILLIKEISKIKGCEPSKIFIEMTRTDEEKGDKGRKNSRAKELTALYKNIKK |
| | EGKAWIELIGKEDASGRLKSKKMYLYIKQMGRDMYTGEQIDLDRLFDDNLYDIDHI |
| | YPRHFVKDNNIENNLVLVNKAKNSRKSDNYPLDSDIVRNPKVRELWEMLHTNHLMS |
| | DEKYHRLTSRKAFTDEDMAGFIARQLVETGQATKGVADLLKELTSSEIIYSKAGNVS |
| | DFRHAYDIPKSRIVNEFHHAHDAYLNIVVGNAYYTKFTSNPLNYIRNEYKKNTKENN |
| | YHLNKIFESTISRNGYVAWLAGSDKNPGTIQTVKKMLSRNTPLMTRMTFVVHGSIAD |
| | ETLYGKNTAKEDSYLPLKGSDPRMSVEKYGGFNKASGAYFFLVEHELKGKKVRTIE |
| | TVPVYLMKKLSDERELQKYCEEKLGLINPSIRLKQIKMQSLIKLNGYFVNVTGRSGN |
| | QVKLRNAVNLILSNKQVEYCRLVEKTTSNNSLDERVNAEANLNLYNVLVAKQGQGI |
| | YQHRPNSVYQKLKDGREVFTKLSLMEQCSVLMQVLNLTIVGQTLGQLTEIGASSKTG |
| | TMKISKRIDLNDTVFLINQSVTGLYQTEVDLLTV (SEQ ID NO: 42) |
| 2183638 | MAQKYYLGLDMGTSSLGWAVTDSEYHLLRAKGKDLWGVRLFPEAEAAAGRRTNRI |
| | SRRRLQREKARIGFVRDVFADAIGKVDPGFYQRLDDSKYFPEDKTEVQRFALFSGNG |
| | FTDREYYDQFKTIFHLRSKLIHSTEPVDVRLVYLAVANIFRHRGHFLNTNLTDGQTAE |
| | LEDIYAQLVDVTSNLPASVDTEKMKEILSSNKITNSRRNEQLMELLQVSKKMPEAEM |
| | LKMICGLKGTLSKAFTDIEFDEDQQKFSISFRDGNYDEKDAEMQTLLDEDSYETVQL |
| | LKQIHDWGLLANIMRGEQYLSDARVKSYEKHAQDLKILKELYKEYGSGQYNKMFRI |
| | MEDHNYSAYVGSVNSDKEKGKVRRGSKKDRDGFYKKIKSDMLGMQKKAPEDDRIR |
| | YILDEIDKESLLPKQLTSANGVIPYQVHLKELRKILSNAEQYLEFLKESDETGLTNSEK |
| | ILQLFSFQIPYYIGPLYNEGNASHNAWVCRKESGKVFPWNFEQKIDVKSSSEEFIRRLV |
| | RKCTYLTSENVLPKSSLLYQKFMVLNELNNLKINGVSIDTELKQDIYRELFCREKKITS |
| | KKIKEYLIMHGVCSKKTGVEISGIDGDFKSSLSSYLLFREILNVSVLSYEQEKMAEQIIF |
| | WATVYGDTKSFLKEKIEESYGDILSPAQIKRICSIKFRDWGRLSKAVLELEGADCETG |
| | EVLTIISRMWNENVNLMECLSDKYTYLEAIRNEGRSISHSFEEIQYEDLEELYISAPVR |
| | RMVWQTILVVKELVKVMGGPPEKIFVEMARDVDGKNEKVRKDSRQKKFADLYKK |
| | CKDEDRDWSKEIADTPEAKFRSKKLYLYYTQKGRCMYTGEPIDLGDLFNDNLYDID |
| | HIYPRHFVKDDSIEKNLVLVKKQVNSHKSDNFPLEADIRTNQYSWWKYLCDGHFITK |
| | EKFERLTRSTPFSAEELAAFISRQIVETRQGTKVVTELFENSFPNAEVVYSKAGVVSDF |
| | RHKFNLLKCREINNFHHANDAYLNIVVGNTYNVKFTKHPANFIKEYQRDPKHFEYH |
| | MDKLFDYPVTRGNEVAWKTKGGESIKTVRKVMSRYTPLVTRMNYEEHGQLWDQTI |
| | YSADDAAKAKGVGYVPVNTNDPKMQDVTKYGGYKKYTGAYFFLVEHLKKGKKVR |
| | TLEAMPLYLKDSLDSKEKMEEYCREKLGYQDPSVRVERIKMYSLIKVNGFFMYLTG |
| | RTGNQLNVINSVEMKLPAHWSAYIKRLFAYSPSSDSDIEESDVKDLVTNERNCTLYSI |
| | LAEKHYSGIYSKRPNPVGTKLKSWEKSFEALPIDKQVYILKQLLQLSSNSNQGADLQ |
| | YLGGAAKTGVALLNKMINGYREFRLVSLSPTGVYKSETDLLTI (SEQ ID NO: 43) |
| 2183868 | MERNYYLGLDMGTSSLGWAVTDPEYHLLRAKGKDLWGVRLFPEAESAAGRRTQRV |
| | SRRRIQRERARINFIREVFSDAINEIDPGFYQRLDDSKYFPEDKTEKQPFALFAGGMFT |
| | DKEYYQRYKTIFHLRSDLLHSGEARDVRLVYLAVLNIFKHRGHFLNANLTDSGTLEI |
| | GEIYAQLLEKTVNLPSDIDLDALKMILSSGKITNSRRNQQLMELLQINKKMPEAEMLK |
| | LICGLKGTLSKAFPEVEFEEEYQKYSVSFRDGNYEEKETEMMSFLDEDCYETVQILK |
| | QIHDWGLLANIMQGEQYLSDARVKAYEKHAQDLNILKSVYKEYGKGRYNKMFRV |
| | MEDNNYSAYVGSVNSDKEKGKERRGAKKDRDGFYKKLKVELHKMQETAPDDDRI |
| | KYLLDEIDKETLLPKQLTSANGVIPYQVHLKELRKILSNAENYLPFLKEKDETGLSVS |
| | EKIAKLFAFQIPYYIGPLYNDGKPGHNAWVCRKEPGKVTPWNFEQKVDVKASSEEFI |
| | RRMVKHCTYMQSENVLPKCSLLYEKFMVLNELNKLKINGIPIDVDLKHDIYRDLFCK |
| | GKRITARKIRDYLVVRGIVSKKADLELSGIDGDFKSELTSYLRFREIFGVDVLTYEQEQ |
| | MAEKIIFWSTIYGDARAFLKERLKEHYGDQLTEAQIKRICGIKFRDWGRLSRAFLELE |
| | GADRETGEVLTLINRMWQENVNLMECLSDRYTYVDVIREEGNGINKAFAEIRHEDL |
| | DELYISAPVRRMVWQTILVVRELVKVMGGPPAKIFVEMARDVNGKNEKERKDSRQ |
| | KKFLDLYRKCKEEGRDWSKEIADTPESRFRSKKLYLYYTQKGRCMYTGEPIDLGDLF |
| | NDNLYDIDHIYPRHFVKDDSIEKNLVLVKKQVNSHKSDSFPIEPEIRKARYIWWKDLC |
| | EKGFITKEKFERLTRSTEFTADELAAFISRQIVETRQGTKTITDIFENSFPDAKVIYSKA |
| | GVVSDFRHKFDMLKCREINNFHHANDAYLNIVVGNTYNVKFTRNPVNFIKEYQRDP |
| | EKYQYHMDKLFDYPVSRGGEIAWKTKGWESIKTVRSVMSRNTPLVTRMNYEEHGQ |
| | LWDQTIYSADDAAKAKGVGYVPINTSDPKMRDVSKYGGYKKFTGAYFFLVEHTVK |
| | KDRIRSLEAMPLYLKDVLDTNDKIEEYCREKLGYIDPSVRMKKIKMYSLVKVNGAFF |
| | YLTGRTGNQLIIANAMELKLPYKWCKYIKRMLTFAYEKDDEDSTISRETNIELFDILN |
| | KKHKNTVFGKRPNSIGKKLSESVDKFKELNINEQVYVLKQILQISSFGNQGADLQLLG |
| | GAAKTGISLVSKKINSQKQFQLISISPTGLYRKEIDLLQV (SEQ ID NO: 44) |
| 2184029 | MMSKYYLGLDMGTATVGWAVTDENYELLRKKGKDMWGVRLFREAETAVARRTL |
| | RVSRRRLQRKIARIGYLKEIFAEEIGKVDAGFYQRLEDSKYYGEDKKENQPYALFTN |
| | RGYTDREYHKEYPTIFHLRKELIESKEPHDVRLVFLSLLNMYKHRGHFLNTNLGDSGI |
| | GSFKDLYFMWQTTAAELMDVNIPKIEDSKWLEEILASRKYSNSGKLYEIIKKFELVKS |
| | VEKQQIEMWKLICGLTSTIATIFQNEQLEEEQKKFSISFRDGNYEEKATQVETMLGEE |
| | SFELFLLLKQLHDWGVLANIMKGEDKTYEYLSFARVDSYDKHGKDLDILQGLYKTY |
| | AKKSQYDEMFRIMKDHNYSAYVGSVNSGKEIIRRGGKCNAEEFFKGLKKQVESFPE |
| | VPDRDYVLKEIEKGSFLPKQLTAANGVIPNQIHKNEMKKILENAENYLPFLGEKDETG |
| | LTYSEKLVRVFEFQIPYYIGPLANGGKNEDSKNHWSVRKEPGQIFPWNFEDKINVKES |
| | AEKFIEKMVNHCTYISGEMVLPKNSLLYEKFTLLNELNNLRINGDKISSELKQRIYCEL |
| | FQGGRKITGKKLEQYLKINGHIDARQEVVISGIDQDFVNRRANYSKFQSVFETEILTY |
| | EQENIAEKIIFWSTIYGDTKKFLREKIQEKYGQVLSETQIKKIIGFKFRDWGRLSRELLE |
| | LEAADKETGEISSVIGRMWNENYNLMEIIASDKFTYREEIVKKQKTIEKTLSEVEYED |
| | LEELYCSSPVRRMVWQTLLILKEIQEVMEDEPSKIFVEMARDPNAEKKRTVSRKRKF |
| | AELYKNCKEDGINWSEEIANREEAAFRSKKLYLYYTQKGRCMYTGERIELYDLMND |
| | NKYDLDHIYPRHFVKDDSIENNLVLVKKEKNAHKSDVFPIESQIRREQYRLWKSLRE |
| | GGFITEEKYKRLNRNDEFSDDERVGFISRQLVETRQGTKLITDLFVRTFPESEVVYVK |
| | AGNVSAFRQQHKFVKCRNVNDFHHAQDAYLNIVVGNVYHVKFTKDAKRFIKEYRK |
| | EPEKNAYHMYKMFDYDVTRAGQTAWRAKSGESIRMVKSIMRKNTPLVTRMNFEVH |
| | GQFSDQNIIGTDQIIKANGKGYLPIKTTDGRLLDVTKYGGFNKMTGAYFFLVEHKQK |
| | KELVRTVEAFPLYLKGKLDTKEKLESYCREVLELNEPCIKLEKIKMYSLLKVNGFYL |
| | YLTGRTGERLLVCNAVQLVLPEKQLNYIKKITKEGIYSEEDYTRLEVTKQQNQELYN |
| | ELEQKHCKLIYAKRPNPIGEKLTKGKAIFEKLPLDRQIYVLLQILQLSQLINQGADLRD |
| | IDEAKGTGVTLIAKRISSNQEIKLINQSVAGLFENEIDLLSV (SEQ ID NO: 45) |
| 2184261 | MSRLVFAYDLGIGQTGWAVVDADSRDIIESGVNNFPSGDAAENQERRGFRQMRRM |
| | HRRKKTRMADFEKLWVSSGFSVPTLSYTDVLSLRNRGLSENLTEDEIFYVLRFMLKC |
| | RGISYLEDELNEDATGEYERAIALNQKELQNKLPCQIQKERMDKYGAYRGEREVEIE |
| | GEKVALRNIFTTDAYKKEIELFLQVQARGNKKITDSFVKSYQEIFSRKREYYVGPGNE |
| | LSRTDYGIYTTIKNPDGTFHTDENLFDKLIGKCSVYPEERRAAAASYTAQEFNALNDL |
| | NDLVINGRKLDEEEKRLIIEEYKSSGVVNVNRILKKAIGENIDSITGFRVDKDGKELFH |
| | TMEIYRKMVKFFVEKGMDITTFSIDELDRIGEILTINTEKDGIIKAFQNSFVKCDANIV |
| | DAFVEFRRKNVRMFTKWHSFSVKIMSELIPEMYAQPKNQMVLLTDMGVFRSRSERFI |
| | ECKEIPIRETTEEIYNPVVRRSVSMAYRITNALIEKYGYPYKVVIEMPRETNSDEEKKK |
| | IGEIQRKNEKELSTIIKKIRDDYGFEIKDEDFRNHKGLVLKLKLWNEQGGKCLYSGKE |
| | IPIDQLVKNQGLFEVDHIIPISISFNDSRSNKVLVYASENQKKGNRTPLSYLSGVNREW |
| | NDEVFTAYVKSLGYGKDKQDYSGKEKLKNLLFAEDITKQEVVQEFVNRNLNDTRY |
| | ASREVLNTLKDYFKAKEAGTIVSVIRGAFTHQMRVNLELSKDRDESYRHHAVDAML |
| | IAFSQLGYDAYLARQKQIIDFETGEILDKASWEKQDSLYRKLVYQETWYQTRIKINEA |
| | EKQVKFYHKVDRKCNRSLCKQTIYGTREYEGDKYVIDKLDLYVNSDVDRFRKMISD |
| | GKEKDFLMYENDPKTFENLMTVFHQYETEKNPFVAYNKETGDFVRKYAKNHNGPK |
| | IEKIKFRKEKLGSCIDISHKYGLEKGSGRVALRSLKPYRMDVYRNKNTGLYRFVGLK |
| | QSDIKCQGNNYVIDEEAYAKILLSEKMIESGQTRTDLGDRGWEFQLSFYRNDIIQYEK |
| | KGVIYTERFLSRTMPEDCNYIETKPIDAPDFKKRNLVGLSQTKSVEKIITSILGNQRNV |
| | KREKFSEIVDTIR (SEQ ID NO: 46) |
| 2184558 | MGGITMDNKYVLGLDIGISSCGYGVIDLETGKFVDYGVRLFKEGTAEDNGKRRDAR |
| | SRRRLTSRRHTRIMDMQKLLKENGIMSNDYHPLQNVYELRCKGLNEKLTNDELTAAI |
| | LHITKHRGSVIETVEEDVKKSDDELSLKATLQHNEQLIKQGKYICQIQLDNLNNKDHI |
| | RGHENNFSTKDYVNELNEILHHQELDEQLINKIIDIVARRRAYYEGPGSEKSPTPYGRF |
| | IEVDGQIKEIDLIEKMRGKCSVFPDELRAPKMTVTADLFNFLNDMNNLTVDGNKLCP |
| | EEKKGILTIVSQKGNITLRQLAKELNVDEVDIKGYRIDKNQKAIFTEFKGYKKIKSILE |
| | KEGYSISLNDYEMLDRIIEILTNKKGIQERKDYLYHLEYRLSDSVVDTLANTTGITGY |
| | HSLSFKALNLLNKELFESEMNQMQLLHELKLFDKNRVSHKGKKNIESDPEAILSPVA |
| | KRAQNETFKVINALRKKYGEFDSIVVEMTRDKNSDEKKKRISNYQKNRENGSKEMD |
| | KFLNEKGYDPTKINGKTRMKISLYLQQGGKSAYTFEPLDLNRVIKDPKYTEIDHIIPISI |
| | SLDDSQNNKALALHSENQVKGNLTPFMAYAANKFNGIGCSYPEFKTNVLSNKNIPY |
| | KKKLNLLNEENITKEEVAKKFINRNLVDTSYACRVVLNTLSDYFKDNEIDTKVHTIN |
| | GRVTDLFRKKIHLEKDRDENYLHHAVDALIVASVKKMNLLNGYLLKQEHDENNLY |
| | NEETGEIITIPDDKVFYDERYIQYIVNLKTLFEESSKYYDHVIEKENMCFNPIKISHKVN |
| | TKPNRQIADETIYSTRNVDGTDYLIEKIPNIYDPKDKKCIRLIQDILNGEDEKYLMERH |
| | DPQTFDLIRDIVKYHYEQFKSDSKMYKVTKKKGEEIITLVGENPLTKYKEENGVIHKY |
| | SKKGNGPEIVSMKYESEKLGNHLDISSNYQTNNKKVILKQIKPYRTDFYQCEDGKFR |
| | FVTVRYTNVSYHKAKGMYVIDEQWYKQEKANKKIKETDKFLFSMHRDELMGIKRK |
| | EGQPYYYDSSTENVDGVTCYHDGITTEMLKFTATNNDTTGKIEVNPLYTYCKKQIM |
| | VGVANLYGVQKYATDVLGNIYEVKDNKLKLEFK (SEQ ID NO: 47) |
| 2184818 | MEGITMDNKYVLGLDIGISSCGYGVIDLETGKFVDYGVRLFKEGTAEENEKRRGARS |
| | RRRLTSRRHTRIMDMQKLLKENGIMSDDYHPLQNVYELRCKGLNEKLTNDELTTAIL |
| | HITKHRGSVIETVEEDAKKSDDELSLKATLQHNEQLIKQGKFVCQIQLDNLNNKDHIR |
| | GHENNFSTKDYVNELNEILHHQELNEQLIQKIIDIVARRRAYYEGPGSEKSPTPYGRFI |
| | EVDGQIKEIDLIEKMRGKCSVFPDELRAPKMAVTADLFNFLNDMNNLTVDGNKLCP |
| | EEKKGILTIVSQKGNITLRQLAKELNVDEVDIKGYRIDKNQKAIFTEFKGYKKIKSILE |
| | KEGYSISLNDYEMLDRIIEILTNKKGIQERKDCLYHLEYKLSDSTVDTLANTTGITGYH |
| | SLSFKALNLLNKELFESEMNQMQLLHELKLFDKNRVSHKGKKNIESDPEAILSPVAK |
| | RAQNETFKVVNALRKKYGEFDSIVVEMTRDKNSDEKKKRISNYQKNRENGSKEMD |
| | KFLNEKGYDPTKINGKTRMKISLYLQQGGKSAYTFEPLDLNRVIKDPKYTEIDHIIPISI |
| | SLDDSQNNKVLALHSENQVKGNLTPFMAYAANKFNGMGCSYPEFKTNVLSNKNIPY |
| | KKKLNLLNEENITKEEVAKKFINRNLVDTSYACRVVLNTLSDYFKDNEIDTKVHTIN |
| | GRVTDLFRKKIHLEKDRDENYLHHAVDALIVASVKKMNLLNGYLARQEHDENDLY |
| | NEETGEIITIPDDKVFFDERYIQYIVNLKTLYQESNQYYNRMIDKEDMHFNPIKISHKV |
| | NTKPNRQIANETIYSTRNVDGTDYLIERIPNIYDSKDKRLVEDILNGNDTKYLMSKHD |
| | PQTFDLIRDIVKYHYEQFKSDSKMYKVTKKKDKEIITLVGENPLTKYKEENGSIRKYS |
| | KKGNGPEIVTMKFESEVLGNHLDISSNYHTNNKKVILKQIKTYRTDFYKCADGKFRF |
| | VTIYFKDVSFNRTKGTYVIDRKWYDGEKEKKRIKSTDKFLFSMHRDELLGVVKEQG |
| | QPYFYDVYDEVPDKKILHDGVNPEILLFALTGNDKTNVVNLYPIFAYKKRQQKVLTT |
| | AKDIRKYATDVLGNIYEVKDNKLKLEFK (SEQ ID NO: 48) |
| 2185070 | MEQKNCKKVDAYNIGLDIGTNSVGWAVTDLDNNILKRGNKNMWGARIFDEGSTAA |
| | QTRILRGTRRRIERRKERINILQSLLLDDMEKEYPNFFPMLRETSKTKEDKNSDSINGK |
| | KYNLFSELELSDPIYYQKYPTIYHLRRELMESKEKHDIRLVYLAIHHIIKYRGNFLYEG |
| | DLKNNNNEIIESINELLDFIQNTLEISFISEAEKIQEILKDKTKSKGEKKDLIIGLFDYDK |
| | DEKSVITGIVNAILGYKFDVDKIFDIEVENSNISFSNEIANEEEIKENLQDHAYAYDVLL |
| | KIYNWFILQDILQGNNSISEAFINKYDKYKKDLSNIKSLYRNYLKDEYNNMFRKEKD |
| | DSYACFEKHTSACPIENLYKRIKKDLADVPDCKEKEEILKDIEDDAFLVKINTTANSAI |
| | PYQLHYQELEKILENQAQYYPTIQENKQHILELMKFRIPYYVGPLAQEGKSRFAWIVR |
| | NSNEKILPWTFDDVVNVDETAEKFIKRMTNKCTYLINEDVIPKQSILYSEFCVLNELA |
| | NIRVNNHKLTPKTKQLIIDKLFKVKKTVKLRDLKQVLTDFQVFPEVTSITGLAEVDKF |
| | MSNMSSYIDIENILGCINDENIEMVENIIEWITIFEDKKILKRKIKNTYNLEDKVVSQLI |
| | KKNYSGWSRLSKELLVGLKAADDGKSIMEKLRTTKDNFMQIINNDMYGFNKQIECR |
| | MKLQEKQITYKDVDEIPTSPANKRAIWQSLKIVEEIKKVMKKDPKNIYIEFAREEQEK |
| | NKRKDNRAKALLKIYEKYEEEIKMLKDYNPNVYKELKKKQNEKDFNERLYLYFIQN |
| | GRCMYSSRPLNIDTLYLYEVDHILPQSYVKDDSLSNKALVYKEENQRKSGSLLIDEKI |
| | INRQETWWKQLRKNGLIDEKKYYNLTRRKMFETDKEKVKFVSRQLIETRQSTKYITN |
| | LLVNQYSNTDVFAIRSNLTHNFRKFFEVYKNRNVNDYHHAQDAYIISVIGNVIDQKL |
| | QYKDEYKYTEYVKKYIKKSEEEANKVWIIMGMVSNNINKEKVKKALEYKDCFITHK |
| | LEEQTGAFYNQTLYSPKDKNIEIPLKEGLDVKKYGGYSGEIKAYFTIYSYIDQKGKMQ |
| | IEMIGIPVKTKYDIENGKTTLIKYIQSKNCEASKIEIIRPKILKYQEYIDENNEPMMLLSD |
| | SEIRTNRQLIVNNEMSKLIYLMNQEKVADDEKEKLQEKMEEIYNYLLGKLKNEYKTF |
| | NSIYQKLSSEETKEVFQKLEYKDKVSAINGIIDLMHRGQGNLTKLKQGDRAGRKSGK |
| | SFKTEALNKMIFIDKSVTGMYERRYKVNGMENCSSK (SEQ ID NO: 49) |
| 2185320 | MGVIIGLDIGIASVGWAVVDRESYKVIESGSNLFTSAEASQNVARREFRQKKRLLRRE |
| | YNRIKDFEVLWMKSGYSIPTGSRNDILQLRVKGLQEKLSQDELFLVLRNLLKHRGISY |
| | LDEALDGADSGNSDYEKGIVKNEQELKEGKFPCEIQLKRLNVYGKYRGRIMIAKDEE |
| | RITLSNVYTTSAYQKEAEQILETQRKEGVGITEDVVQQYMMIFSRKRKYYEGPGNEK |
| | SRTDYGKYTTRINPETGEYVTEDNIFEKLIGKCSVKPNLMRAAGATYTAQEFNVLND |
| | LNNLMVNGRKLEEMEKIEIVEAMKSATSVTVNVEKIIRQVIGTDILSLTGARLDKDDK |
| | PEFHQFELYRKLKREFGKCNIDIDKFSIEQLDKIGNILTLNTEKEGILQGFVRQELILTE |
| | DEKECLIQFRKKNGKLFNKWQSFSLEVMQELIPDMYEQPKNQMELLTEKKMFQSNM |
| | EKFKDCTRIPSACITEKIYNPVVCRAIRVTISIVNALKKKYKSIDQIIVEMPRDRNEDEQ |
| | KKRITKAQKTNAKELENIIKKIKGEYGITISNEEFRGHKKLALKLKLWNEQEGKCLYS |
| | GKAIEILDLLHNPNMFEIDHIIPLSISFDDSRSNKVLVYGTENQEKGNLTPFMYLQRVN |
| | RDWDFDEFMSFVLELKERKSISKAKVDKLLEYRDITKVEVLKGFIARNINDTRYASR |
| | VVLNTLQEYFKAKGANTKVKVIRGNFTHQMRNALYLEKNREASYAHHAVDAMLLC |
| | YSQMGFDAYHKLQEKFIDFEQEIILDTKVWEQQMDSRTYKEVMYQDKWMTIRKNIV |
| | DAEKRIKYWHKVDKKPNRALCNQTIRGTRQIDGKIMKINKVDLYSKNGWKTLKKLL |
| | DSGKEERFLMYEKDQRTWTDMLKIIEEYRDAENPFVEYERETGDYLRKYSKKHNGP |
| | RIVQLRYTDGEVGSCIDISHKYGFDRGSQKVILEKLNPFRSDVYYNRQEQKYYIVGIK |
| | YSDLKFEKGKYVISEEAYNQILVQEKMMSVSDTRLDLERLGYEFCFSLYKNDFIEYE |
| | KDGKYFTERFLSRTNPKAKNYIETKPIAAAKYEKQRLVGLSKTKSIKKIYVDILGNRY |
| | YCKKEKFIKEVDI (SEQ ID NO: 50) |
| 2185440 | MYSGAGGIFDVKKIGGNVMNYVLGLDIGIASVGWAVIDLETLQVIESCSNIFTAAEAE |
| | NNKDRRSFRQMRRLKRRLRTRISDFAKMWKSLGFSIPGDIDNMPLLLRVKGISEELS |
| | MDEIYTVLLNELKHRGISYLEDFGDNANDGTDYSKSIAINQKELEEKFPCQIQYERYC |
| | KYGNYRGQFVADINGEKVTLCNVFTTSSYRKEITSILDTQKKFHSEITDKFIENYMKIF |
| | DRKREYYVGPGNDNSRTDYGIYTTKLNDDGTYKTDKNLFEKLIGKCSVYNEELRAA |
| | AASYTAQEFNVLNDLNNLIVNGRKLEEDEKRKIVDTLKTSDSINMRKIIKKIIGEDIET |
| | LEGARIDKDEKEIFHCFEIYNKLRKELKKIEYDINLLSRDTLDDIGRILTINTERDMIVN |
| | SLSELGLDNDVVNCLADVRKKNGKLFSKWHSFSLKIMQELIPAMYEQPKEQMQLLT |
| | EMGVFKKKGDLFKDCQYIPVDIAKDDIYNPVVVKSIHIAIRIVNAIIKKYGMPEKVVV |
| | EMPRERNTEEQKKRIKDTQRKNEKELEEITKKIYAEYELRIEDKDFRNHKGLALKLKL |
| | WNEQGGVCPYSGRKIDIFDLVNHPDMFEVDHIIPLSVSLVDSRINKTLVYKVENQEK |
| | GVNTPYMYLSKITRDWGWEEYKNYILGLKIEKAKKANYLFMEDITKYDVLKKFINR |
| | NLNDTRYASRVILNTLQDFFSTKQTGTIVKVIRGSFTHQMRVNLHIEKDRDESYSHHA |
| | VDAMLLCYSQLGYDAFHKLQGSFIDFETGEILDKRMWNENMQEDVYKDYLYGNK |
| | WSQIKRHIEDAEKKVKYWHAVDSKCNRGLCNQTIYGTREIDGKIYKISRTADIRTKS |
| | GFESLKKLLAESNGKSKKSDILMYRNDPKTFQILMGIMKQYADATNPFVKYEEETGD |
| | VIRKYSKKHNGPKVTKISYMDGEVGSCIDISHKYGYAKDSKKVILESLVPYRMDVYY |
| | NITDKLYYFVGIKQSDIKCKGDEEIVNMEAYRKALVSEKMILPEQTIDDLEKLGYEFR |
| | LSFYKNDIIKYEKNGELFTERFLSRTMPAVRNYIETKPVDAPKFEKQHLIGLSKTKMV |
| | KKIRTDILGNCYECEKEEFSFICK (SEQ ID NO: 51) |
| 2185446 | MGIRFAVDIGVASVGWAVVNDEYVVLESGVNIFPCADASKNAERRNFRQLKRLHRR |
| | RKNRVSDFEKLWQEENLEIPKNNQINVLQLRVKGLSEKLTQEELFWVLKNELLHRGI |
| | SYLDDALEEGAANESDYKKALERNQTELTQNEYPCIIQQKRFESYGEYRGNRSFEKD |
| | GEIISLSNVFTTNSYKKEIQAILKRQKEFHLFLTEKFQDKYIEIFSRKRKYYEGPGNEKS |
| | RTNYGKYTLRINPKTGEYITEKNIFEKLIGKCTIYKDQVRAAGASYTAQEFNLLNDLN |
| | NLTVNNRKLTEEEKRNVVNEVKTAKSVNVKRILKKVIGEDIETLTGLVLDKNDKEKI |
| | HSFEQYNLMRRAFEKNGWDIEKYTREELDRIGEILTLNTEKEGIIAAFQNKDDQDDFK |
| | MINISDEQIECLIELRRKNSAKFSKWQNFSLKLMKEIIPDLYENSKNQMQILTEKELLK |
| | NNKEKYKEYKYLPEEDIIEQIYNPVVVRAIRITIRVVNALLKKYGSPRQIIVEMPRDKN |
| | EEEQKKKIKDAQNKNKKELDDIIKQVKEEYGIAITEQHFYHHKKLGLKLKLWKEQNE |
| | QCLYSGKKIEIYDLLNDQKKFEIDHIIPRSISFDDSRSNKVLVYSSENQQKGNLTPYLY |
| | LRGLKREWNFDRFKAVVVELNKKKSITKNKLDKLLCMENITKQDVLQGFVERNIND |
| | TRYASRVVLNALQDYFYSHEANTSVRVVRGSFTHQMRVALELNKDRDESFVHHAV |
| | DAMLMCYSQMGYETFHRQQKNLIDIETGEILDVEEWDKLMDDEKCTEIMYQQKRM |
| | RIIREIKVAQSRVKYWFKVDKKPNRGLCNQTIYGTRNVDGETYKINKLNLYNAEDVK |
| | KIRQMIAENQQSKFLMYDLDRKTWDGLVKILEEYSDSRNPFLDYEKETGDAFRKYA |
| | KKNNGPKVVKLKYKKEKVGSCIDISHKYGFEKGSRKVLLDSLTPFRTDVYYQPETKQ |
| | YFLVGLKHSDFKFEGGHYLIKEEVYNQNLIKEKVIQTGQTWKDLGKMGIEFCFTFYR |
| | NDIIQYEKNGEVYTERFISRTKKNLNYIEKKPLKKPDWGKEEKRYRTVGLVKTCSIRK |
| | IRTDILGNQYVCEREKFRLEVDC (SEQ ID NO: 52) |
| 2185499 | MEQKNLKKYPEYSIGLDIGTNSVGWAVTDSENNILKHGGKNMWGARLFDAGDTAA |
| | QTRTFRGTRRRIERRRERVNILQSLLLDDMEREYPNFFPMLRESSKVEDEKQQEKLD |
| | GKKYNLFSELNFSDQDYYAKYPTIYHLRKELTTSKEKHDIRLVYLAIHHIIKYRGNFL |
| | YEGDLKSNTDEVLESIEFLVNFIRENFEIEFNSTSEKLRNILKDKETTKAEKKDALMRL |
| | FDYSKEDKAIINGIICAILGYKFDINKIFDVELENSNISFSSDIANEEEIKDALQENADVY |
| | DVLQKIYNWFVLQDILQGNSSISEAFIKKYEKYKKDLENLKNIYKTYLKTEYNNMFR |
| | QEEIGSYAMYDKNISKCPIEELYKKIKKDFASIPDCKEKQEILQEIDNEAFLIKINTTAN |
| | AAIPFQLHYQELEKILENQAKFYSTINENKENILNLMKFRIPYYVGPLAKNDNSRFAW |
| | IVRKSDDAIRPWNFEECVDVDATAEMFIRRMTNKCTYLLSEDVIPKQSILYSEFCVRN |
| | ELANVRVNNHKLSPQTKDLIIEKLFKMKKTAKAKDLKQILIDNQIFKKISSVTGLAEV |
| | DKFMSNMSSYIDMKEIFGIVDENSIEMIENIIEWITIFEDKKILKRKIEREYHLDEKIINR |
| | LAKKNYSGWSRLSKELLIGLKSFDDGKNIMEKLQSTKDNFMQIINNDQYGFNKQIED |
| | RMKNNDTKITYEMVDEIPTSPANKRAIWQSVKIVKEITKIMKSEPKNIYVEFAREEQK |
| | KKVRKDNRAKALLKIYDTFAEEMKMLRDYNPKVYQELKRKQNDRDFNERLYLYFT |
| | QNGRCMYSSRPLNIDTLYLYEVDHILPQSYVKDDSLSNKALVYKEENQRKSGSLLLD |
| | EKIINKQELWWKQLHKNGLIDDKKLRNLTRRKMYETNDDKVKFVSRQLIETRQSIKY |
| | ITNLLVNQYKDSDVFAIRAELTHNFRTFFEVYKNRNVNDYHHAQDAYIISVVGNVID |
| | TKLHFKDEYKYTEYVKNYIKKNEEEQSNKRKVWIIMEMVSNNIDKEKVKKTLNYKD |
| | CFVTRKLEEQTGEFYKQTLYGPNDKNVKPVIPLKKGMNVEKYGGYSGENKSYFTIFS |
| | YIDKKNKKQVEMIGIPVKTSYDIKNNKITLKEYVESQIDNTTEVKIIKPKILKYQEYLD |
| | ENNESMVLLSDRELRTNKQLVLPSKVNKLIYLMNKEKATEDEKIEVEMNIEEVYNYL |
| | IKKLNDEYKSFSSIYEKAKDDKTKNKFNEMSYDDKVVTINGLLDLMNKGQGNLSKI |
| | GLGDRAGRTRKSAFKTKDLENMTFIDKSVTGIYERRYKVNGMENGSSK (SEQ ID |
| | NO: 53) |
| 2185882 | MKVIDKYYVGLDLGTSSVGWAVTDDKYQLLRAKGKDLWGVRLFEEADVAAGRRV |
| | NRVSRRRRQREVARIGLLKEYFAEELSKVDSNFLERLAESKYHREDKKVATKYGIFA |
| | DDNYTDEDYFREYPTIFHLRYELIQNPQAHDVRLVYLALLNMFKHRGHFLNAGLKT |
| | GGETESMEDIYGLLYMTAQEWEIELPETVDFIKMKDILSRRDISRTVMTEQLSEAFAIE |
| | KKKNKQVYEILKGFCGLAVSLNTLFSREEFDENHKKVQISFRDSKYDEKIEEYSSFLS |
| | EEEVELIGRIKQIHDLGVLSNVMKGSKYLSEARIREYDKHKQDLKILKRVIREYSPDK |
| | YDSMFRELEPGSYSAYVGSVNSGESHRRNVKNRQREDLYATIKKILKDIPDNEDKEYI |
| | LKEMELEAFLPKQLTASNGIIPNQVHVIEMKKILSNAEGYLPFLKETDESNLTVSERIL |
| | QLFEFQIPYYVGPLNLQHKNMPNGHAWAVRKKKGRVLPWNIEEMIDIKQSSEEFINR |
| | MVRHCTYLQGERAIPKDSLLYERYCVLNELNNLQIHGERISPQLKQNIFCTLFEKGKH |
| | VTQKQLFQYLVREGILEETELDAISGIDKDFKNALTSYGKFKAVFGESIKLDSTKKMV |
| | EDIIYWGTIYGDDKKFLREKIKDEYGECVTEEQLRRIAGFKFHDWGRLSRAFLQLSGC |
| | EKGIGEKLSLIDMMWENNYNLMELMSDKFTYQDSLAEMCENSEKVLKELEYEDLED |
| | LYLSAPVKRMIWQTVLVLKDIEHVLGKEPSRVFVEMPREDGDKGVRKDSRKQKLKE |
| | LYKGCKVDSKNWIEEIDGWSDQELRSKKLYLYYMQQGRCMYTGEIIDLHDLFNDNL |
| | YDIDHIYPRHFVKDDSLENNLVLVKKEVNAHKSDTYPIEKSIYDARHNWWRSLLTAD |
| | MQSKFITREKYNRLVNRNEFTDEQLTGFISRQIVETRQGTKVVAHLFEEMFPTSEVVY |
| | VKAGNVSAFRQERKLLKSRVVNDFHHAQDAYLNIVVGNTYFVKFTRNPANFIREYR |
| | KNATANRYHINKMFDNTVERNGEIAWVAAEKNGASGTIQTVRAVMAKNSPLITRM |
| | NFESHGQIAEATLYGAKNANEESYIPLKESDERLQDVSKYGGENSISITYFFLVEHTKG |
| | KKRIRTIEAMPLYLKDKIGTDKKALVKYCEEQLGLIEPDIRLEKIKIQSLIKRNGFYLYI |
| | SGKSNKQLIVRNAVPLCLKQDWITYIKGVEKVNETGREYSLVTAEKNLELYEMLMD |
| | KHMTGVYAKRPNPVGEKLVHAKDRFEQLEVKEQCKVIMQILQLSQLSNMGADLSLI |
| | GEAKKTGICMISKNVSEADEFKLIHQSPSGLFETEIDLKTI (SEQ ID NO: 54) |
| 2185938 | MEKFKKYNIGLDIGTTSVGWAVVETENQKILRKGNKPLWGIRLFEEASKAEVRRNFR |
| | STRRRYDRRRQRISLLQEEFKEEINKVDSNFYIKLQESKYVTSDTINKTIKITDSEKEKL |
| | KKYLKECPTIYHLREKLINNTEKEDIRLVYLAIHHIIKYRGNFLYNSKGFNVNNLNIEE |
| | KIKEAFDTYFSYINTEYDDYSSLLDIKKLSEALMQQSKKDAREEIKNVLTESGLFENKI |
| | VNEFIKMMIGNKFKITTLLSLEDSDSKLEISFDGTEFEDKYDEIESITLDKIEILETMKEL |
| | YDTLFLKKLFKGSNETNISSLMVKKYNEHKEDLKFLKKILKSDKELYKKFFKNDCIY |
| | EKYITNNITYDEFIKEVNNAISKVNIADYNLSNEYVTKIAPEIANGNFMPRITSTENGK |
| | YPYQLNKDELLKIIENQGKHYPFLLNKTNDGTYKIVKLLEFRIPYFVGPLVNSNQSKF |
| | AWMERNIQNVKITPYNFNEVINKELTAEKFIKRMISNCTYLLNEPALPNNSISYNNFK |
| | VLNELKQIRINGELLEINLQHKLIEELFKKTNGTITEKKFKDYLLSNNEFPMHGTELNI |
| | TGYSADGAFANNMQSYIDFFGKNGIFEDTSYTINDAENIIEWITIFDDKDILEQKVRKK |
| | YPKLNDLKIKSLLSKKYKGWGSLSSKLLNTKYYIDKKSNTKKSILDLMYETKENFMQ |
| | IINNDEYNFQQMINEINNTNNDIEKLSYKVVEPLATSPANKRGIYQALKIVDEIVNYIG |
| | YEPESIAIEMARNEDKKERKDNKKKYIENLYKKSKDEIENYNKLQKELNQEEINSQK |
| | MFLYFIQEGKCLYSGKSLNIEDLDSYEIDHIIPRTLIKDDSIDNKALVYRECNQNKAAS |
| | YVLPQEYRNKYNQEWWKHLRKIGLMSAKKYHNLIRKEYSEEDIEGFINRQLVETRQI |
| | TKHVANILNTYYKNTKVIYLKAKLSHDYRDRYDLFKFREINDYHHAHDAYLAAVLG |
| | EYKEKYLNKDVNFEVVKELNYMLINSGNKDKIKYGYVINSLNEQAHDLIFELSKKY |
| | MNEETGEVIFNAEEFNKRIEDTLYRNDILVSKKTEIRTGEFYNQTKNSKGNKGIELKK |
| | NMPTELYGSYTSLNPAYAIMVKYTKKNKTSQKLIGIPIYLTKQNDETIKDNYIRNILNI |
| | SMNDSYEFTSKPIPFFSKLNWNGQICYLVGASDTVEVCNAQEFNYDKEFFKYHKLAL |
| | QKLFNNKNLEINDNIYSNMLDEIIKYIVDKIEKQYKLFGNLIFELKIMINYDNIESNSVE |
| | NKENIIKQLTKLLNCKSDNANFKFLNNKFSSAFGKKNGRTIEKFSLINLSVSEIYQKETI |
| | IGE (SEQ ID NO: 55) |
| 2185986 | MRYVLGLDIGIASVGWAVIDLETLKVIDACSNIFAAAEAENNKDRRSFRQMRRIKRR |
| | MRTRISDFAKMWKLFGFNIPEEVNNMPLVLRVKGINEELSMDEIYAVLLNELKHRGI |
| | SYLEDFGDNTNDGTNYSKSLEINQKELEDKYPCQIQYDRYCKYGNYRGQFVADING |
| | EKVTLCNVFTSSSYRKEIIKILDTQKKYHSEINDEFIEKYMDIFNRKREYYVGPGNEYS |
| | RTDYGIYTTQLNEDGTYKTDENLFEKLIGKCSVYSEELRASAASYTAQEFNVLNDLN |
| | NLIINGRKLEEKEKYKIVDIIKSSDSINMRKIIKKAINEEIETLEGARIDNNEKEIFHCFET |
| | YNKMRKEFKKIDVDINSLSRDELDEIGKILTINTEKDMIVKKLSEMGLDDKVVDCLT |
| | NIRKNNGKLFTKWHSFSLKIMNELIPTMYEQPKEQMQLLTEMGVFKKQSDLFNNCK |
| | YIPSDIVKDDIYNPVVAKSVHIAIRIVNAVIKKYGMPEKVVVEMPRERNSEEQKKKIK |
| | DIQNKNKNELDGIIKKIDSEYDLKIGEKDFRNHKGLVLKLKLWNEQGGVCPYSGRKI |
| | DIFDLVKHPDMFEVDHIIPLSVSLVDSRINKTLVYKVENQEKGVNTPYMYLSKITRD |
| | WGWDEYKDFIHGLKIEKAKKVNYLFMEDITKYDVLKKFINRNLNDTRYASRIILNTL |
| | QDFFNANKTGTIVKVIRGNFTHQMRTNLNIEKDRDESFSHHAVDAMLLCYSQLGYD |
| | AFHKLQGSFIDFETGEILDKRMFNENMQEDVYKDYLYGNKWGQIKHHIEDAEKKVK |
| | YWHAVDTKCNRGLCNQTIYGTREIDGKTYKISRTADIRTKGGFDSLKKLLAEKNCKS |
| | KINNILMYRNDPKTFQFLMDIMEQYSDASNPFVKYEEETGDIIRKYSKKHNGPKITKIS |
| | YIDNEVGSCIDISHKYGYEKASKKVILESLVPYRMDVYYNTKDERYYFVGLKQSDVK |
| | CKGNEKIINTENYKRSLVMEKMISDDQTIEDLEKQGFKFKLTFYKNDIIKYEKDGRIY |
| | VERFLSRNLSARNRIEIKPINAEKFDKPQVFVGLSNTKMVKKIRTDILGNYYECEQEK |
| | FSLICK (SEQ ID NO: 56) |
| 2186029 | MGRLALGIDEGIASVGYGIIDLDMLEFVDYGVRLFEESTAADNKTRRTTRGHRRLISR |
| | KVTRLDDMKHLLKSLGILTDGFHPLNNVYEIRAKGLKEKLSNDELVAAILHITKRRG |
| | STIETADDTEEASKETGELKEILQANSKKLAGGKFVCEVQLELLNNGKSIRGHHNNFK |
| | TKEYVKELKEILSHQELPEEACEAIIQIVSRRRAYYEGPGGRKSPTPYGRYIEANQGEP |
| | IDLIEKMRGKCSVYPDEPRAPKLAVTSDIFNLLNDLNNLAYDGDNKITMDQKDKVFK |
| | IVDKKAKVEPKDIAKIFGTDLSNISGFRIDKKKKPLKVEFKGYAKLKKCFKDCGETISL |
| | KDYEVLDQIIEILTNRKGIEERKQLLNLVKSSLITDKSVELLANLNGISAYHSVSLKAM |
| | RELNKEMYVTNYNQMQLLQQIHKGNDVSALKGKKYIEADDTAILSPVAKKSQRETF |
| | KVINELRREYGEFDAIVVEMAREKNSLDKKKRITERQKFFEEKNKEVDKLLKESGYD |
| | PGEINGKTKMKIRLYLEQDGKSAYTLEPLILKEVINDPDYTEIEHIIPISISLDDSYSNKV |
| | LATAAENKVKGNRTPIDAFNKHLFNDSDNGLHGDLQLYISSVKSSKKYSKKKKRYL |
| | LFKDDITKMDVIQKFIARNLVDTRYANRVVLNTLTDYFKANDIPTKVFTINGNITNIFR |
| | KKIHMTKDRDADFLHHAVDALIAASVKTLGLLNGYLGKYDLYDLYDEVTGEVKKV |
| | PDEDEFFDEKYITFISALKNIYDESSKFYRGIMTRNQLAYAPIKISHKVDTKPNRQVAN |
| | DTIYSTRIVDGEEKRVMKFKDIYDPKFKELTEDVINNAAENKYIMAKKDPATFEEISRI |
| | ILDHYETFKDDPKQYVMGKDGVKLKGENPLTAYKEEFGKIRKFSKKGNGSEVTSIKC |
| | YYETLGNCIDISKNYNLTQGKRVVLVQVSPYRTDFYRDVDEKIKIVTIRYKDVFFKEN |
| | LGLYCIDHDWYKSEKSKKNISDKAVFLCSLHHNELIGIVKKEGQKYIYDDSRESDGN |
| | TKCHDGITPEILKFTATANDLENKIEVKPVYKKAKSRKSPSVGTFINVKKYATDVLGN |
| | LYEIKDNNLKLEFK (SEQ ID NO: 57) |
| 2186035 | MEQKSLKKLIDYNIGLDIGTNSVGWAVTDFDNNILKHGNKNMWGARLFNEGDTAE |
| | QTRTFRGTRRRIERRKERINILQSLLLSDMEREYPNFFPMLRETSKVKEEKKEESLNGK |
| | KYNLFSELKFSDATYYAKYPTIYHLRRALIENNEKHDFRLVYLAIHHIIKYRGNFLYE |
| | GNLKNNSNEILESIDELLNFIQNTLEIVYVCRAEDIQNILQDKTKSKGEKKDDLMSCFD |
| | YEKDEKTIINGIICAILGYKFDVNKIFDTNIENSSITFSGEILNEEEIRDCLQDQAYVYDV |
| | LQKIYNWFILQDILQGNASISTAFVNKYEKYKKDLQNLKSLYREYLRNEYNDMFRKE |
| | NNSSYASFDKNVSSCPIENLYKRIKLDLEKIPECKEKNDILKEIENQNFLIKINTTANAA |
| | IPYQLHYQELEKILTNQEPYYATIRENKQHILELMKFRIPYYVGPLAKEGESRFAWVV |
| | RNSKERILPWTFDDVVDVDATAEKFIKRMTNKCTYLINEDVIPKQSLLYSEFCVMNE |
| | LANIRVNNHKLSSKEKKLIIDKLFKVKKTVKLKDLKQILLDYQVYSEITSITGLAEVD |
| | KFMSNMTSYIDMENILGEINETNIEMVENIIEWITIFEDKKILKRKIKKEYNLEDNIINQ |
| | LSKKNYSGWSRLSKRLLVGLKSYDDGKNIMEKLQTSKDNFMQIINNDKYGFNKQIEE |
| | LMQIQEKEMTYEAIEEIPTSPANKRAIWQSLKIVKEITKVMKKEPKNIYIEFAREEQEK |
| | KKRKDNRAKALLKIYDKYEEEIKMLRDYNPNVYKELKKKQNEKDFNERLYLYFTQ |
| | NGKCMYSSRPLNIDTLYLYEVDHILPQSYIKDDSLSNKALVYKEENQRKSGNLLLDE |
| | KIINKQENWWKQLRKNGLIDEKKYFNLTRRKMFETDKEKIKFVSRQLIETRQSTKYIT |
| | NLLVNQYPKTDVFAIRSNLTHNFRNFFEIYKNRNINDYHHAQDAYIISIIGNVIDKKM |
| | HYKDEYRYTEYVKNYIKKSEEELNKNKTWMIMGMVSNNIDKSKVKSTLCYKDCFIT |
| | HKLEEQTGAFYNQTLYGPRDKKVTAVIPLKDNMSVEKYGGYSGENKAYFVIYSYTD |
| | KKNRQQTEMIGIPVKIAYDIKEKKITLSKYIKNKIKDGSNIKIIKSKILKYQEYLDENKE |
| | PMMLLSDSEIRINKQLVVTPEINRLIYVMNQEKTTEEDRKEIEEQIENIYDYLLDKLKK |
| | EYRFFNSIYTKLNAPETKELFEKLEYQDKVLTINGIIDLMHKGQGNLSKLKQGDRAG |
| | RKSGKTFKTETLNNMIFIDKSITGMYERRYKVNGMENSCSK (SEQ ID NO: 58) |
| 2186149 | LKNEKNYNIGLDLGTGSVGWCVTDENNNIVKHNSKNMWGARIFEEAHTAETRRNFR |
| | SARRRADRRKERINILQSLLKDDMDKEYENFFPMLRNTYLVPEEKGFEYEMNGKKY |
| | NLFSDSNFTDENYYKKFPTIYHLRNYLIETTEKVDFRLVYLAIHHIIKYRGNFLHEGNF |
| | SNDNNSINESLIELTDFLKEKYDISLINDNEDIISILLNKNIKRQNKKQELLKMFDFDKE |
| | DKKVITNIVNAIFGYKFELSTIFDIEYDGKIDFSTEIVDEDSIIENLNEESSIYEILKSIYS |
| | WSVLQEILNYDDSANEENKKYISKAFMKKYDDYADDLKCLKEFYRKYLPKNKYKE |
| | MFKIYDEKKNNYVAYNGKNKGESNKKCDRYALYSYIKNDIDEVKESTDKKYIIDKM |
| | DNCDFLKKLNVTDNGAIPYQLHRDELLKIIENQEKYYKTLRENKTKIKDLLEFRIPYY |
| | VGPLEKNEENCNAWVVRKSNEKIRPWNFDDVVDKDATAEKFIRKMTNKCTYLINED |
| | VMPKQSILYSKYCVLNELNNIKLDENRISKDTKKKVFCRLFLTKTKVSANDIKKLLA |
| | QDGLVIDNITGFTDESSKVFNSNMKSYIDLKSIIGDITENNFDDCEKMIYWVTIFEDKSI |
| | LKKKLKENFEYLSDKQINKISKLNYSGWSRLSKKLINGLKSYDKNETIMEKLENTNM |
| | NFMQIINKDEFGFKKQIEDFMPKTEKGIHYKDIEDIPTSPANKRAIWQAMLVVKEIVK |
| | VMKCEPQNIFIEFARNEGKKGEITKKRFNKIIDIYNEIDNIKKNDELYKELKSHQSDKE |
| | FSERLYLYFIQLGKSLYSGKPIEINRLSEYQVDHIIPRSYTKDDSIDNKALVYADENQR |
| | KADDLLLDSSIQRNMRSWWEYLLKNGLISPAKFNRLTKTKMFTNDDDRSKFVKRQL |
| | VETRQITKYVTNILNNNYKNTNVFSLRSNITHNFRNQYKIYKNRNVNDCHHAQDAYI |
| | MALTGNILNKEWKNINEFKYGKYVKDYMNDKNFSKNKNGIIVGFISKYVDAGKLRK |
| | VMEYKDFYISTMLMEQTGAFYKQTICSPKKHPTISLKNGLDTSKYGGYTNEFKAYCA |
| | IYKYLDKKKIERYRMIGIPVKIASDIKNNKITLQQYLEEYSKEKNEVFVNIFKSKIMKY |
| | QEYLDENGNKMILKSDSEIKSNKQLILSLDMNELIYAINNDQADKEEYLNKYGNVFE |
| | FVYDKLAEKMHIEYPIFETNYKKIIESKNTFNELDLKDKKDTINGIINLMSGEEGNLTK |
| | IGLGDRTGRMSKQKFEDKRVRNIIFIEKSVTGMYERRYKIDGMENSCSN (SEQ ID NO: |
| | 59) |
| 2186167 | MGVRFGLDIGIASVGWAVVSDEYQILESGANLFEAAEASKNADRRTYRQTKRLLRR |
| | RRTRVKDFEKLWIKYGLPVPEHMCNNQLDMRIKGQHEELSKEEIYFVLKYMLLHRGI |
| | SYLDDVVDDTNVTGEYKKGLLYNQKELNENKLPCEIQYERLCKYGKYRGEITTTNN |
| | EEKITFSNVFTKSAYKTEITNFLDMQKKFHPFISAGFISEYMVIFERKREYYEGPGNEK |
| | SRTDYGKYTTLINPETGQYITEENIFEKLIGKCSIYTEERRISAASYTAQEFNLLNDLNN |
| | INVNNRKLSKEEKCAIICKVKTEKVVNMKRIISDVMGEKIEYLTGCRIDKNGKEIFHK |
| | MEQYNKIRREFEKVGLDVDYFSRDELDQIGDILTLNTEKEAILCAFKRNGVVMSTEM |
| | QETLMEIRRKNASMFSKWHKFSKKLMLELIPELYEQPKNQMQLLSEMGVFKLDTEK |
| | YKQYSKLPKEEILDDIYNPVVRRSVSVTIDIINALIKKYGWPKEIVIEMPRDRNTEEQK |
| | QRIRDFQKNNEKELKAIIKKVKDEYGYNITEDHFYKHKKLAMKLKLWNEQDGLCLY |
| | SGKTIDIYDLVNNRNAFEIDHIIPRSISFDDSRTNKVLVYATENQLKGNQTPYMYLRET |
| | KKMWGLDDFVDYCCTLKDKKKISKTKLDKLLFREDITKIDVLKRFISRNINDTRYAS |
| | RVVLNSIQSFIKAKESETKVKVVRGSFTSQMRMAMQLPKDREKDYSHHAVDAMLIC |
| | FSQMGYEAYHLIQKEFIDFENEEITNYSLWSKNMDDATYQKVMFSQRWKLIRDNILN |
| | AEKKVKYWHRVDKKANRQLCRQTIRGTRRIDDKNMIVYKLNIYDKTGYDTLKKKL |
| | SNGKESDFLMFHNDPKTWEQLKMVLNEYSDAKNAFVQYELETGDCFRKYSKKHNG |
| | PKVTNLKYISGEVGSCIDISHKYNLSPNCQKVVLENINPYRTDVYYSQKEKRYFLVGL |
| | KYSDFKYTSEGYRIDEKAYCNALKNEEMLQSGMSLKDLSRQGFEYLFSLYKNDYIEY |
| | ELKGKVYVERFLSRTMPQKANYIETKPIECANYEKRHPVGLKKAISIKKIRTDILGNR |
| | YYAQKEKFTLDVDN (SEQ ID NO: 60) |
| 2186199 | MEKEYYLGLDMGTNSVGWAVTDDAYNLIRFKGKDMWGIREFDEANPAVERRTHRT |
| | SRRRKQRNAVRMGLIRSYFADEIQKVDSNFYQRLDNSFFHVEDKQDNVKDKNGIFN |
| | DKNYKDADYYKQYPTIFHLRKELIEDASQKDVRLVYLAVANMFKHRGHFLLSGIGS |
| | ESSKQTITDLYNQVSGLITEEYGISLKTDIAGTDFEQVLCSREYSRTRKSEKVCEILEIK |
| | NSDKLKKAFVKSMCGLSVKITDIIDDTFTEISDEDKKITFSFVDSDYDEKAGVLEEKL |
| | GTDVYDIISKLKEIHDKAQVAEIMKNYQYLSFARVADYEKHKKDLKILKRVVKKYFE |
| | PTVYDKLFRSEENGTYSAYVNSFNSEEKSRRNYKERKQEDLYNSIKKLLKSVDNVDD |
| | TDIEYIKNEMDKENFLPKQLTFKNGVIPNQIHASELKAILKNAEKYLPFLNEKDESGY |
| | TVSERILMLFSFQIPYYVGPVSSNSNGWVKRLEPGQVLPWNIDKKIDLKETSEQFIQR |
| | MVRNCTYIAGEKVLPKASLLYENFKVLNEINNLSINGERITVNLKQDLYNTLFLKGK |
| | RVTRKQIFNYFHSRGLVEEDSQISGIDIAVNNSLSTYGKFLPLFGEHLKEDKYKKMVD |
| | DIVFWCTVYGDSRKFLREQLEEKYSDVLDENQIKRITGMKFKDWGNLSKEFLELSGC |
| | CKSTGEVMPLIRAMWETNYNLMELLHSEEFTYSEELEEKQGKIEKVLSEYTIDDLNE |
| | EYFSAPVKRMVWQTLLIIKEIEKIMKCPPTRIFVEMTRKDDEKGDNGRKSSRKDQLL |
| | ALYKKIQSEDKEWKKEMSSRIEDYDKNGALRSKKLYLYFIQKGLDMYTGKPIDLDN |
| | LMSANSNYDIDHIYPRHFVKDDNINNNLVLASKPANAEKSDIYPIKDSIYQKMKYTW |
| | KVLRDEGLITEEKYKRLTGRNPFTDEQKAGFIARQLVETGQGTKGVADLLTALLPDT |
| | KLVYSKAGNVSDFRKRYDIPKSRLINDFHHAHDAYLNIVVGNIYFTKFTSNPLNYIRK |
| | ECSSKDNKYQYNLGNMYKWDVKRDGKTAWVAPEEGETGTLATVKKMLSRNTPILT |
| | RLTIEGHGGIADATLYSHKKAKQDTYIPLKSSDARMADVTKYGGYSSVTTAYLFLVE |
| | HEIKGKKIRTLETVPIYLKDRLENSKEKLEEYCRETLGYTNPSVRMKKIKLQSLIKLNG |
| | YYMQISGRTGNRFTLRNYVQLCLNGKWVDYIHKLEKYQDKGVIDDLITDERNVELY |
| | NVLCEKHSVGIFTLRPNLVGDKIKNGYDKFVNLTTEEQIKLLIEIIKLTCVGIVSANLTL |
| | IGGAAISGKMLMSKNITYANECILINQSVTGLYENRIDLLRV (SEQ ID NO: 61) |
| 2186215 | MKYYIGLDMGTSSLGWAVTDDKYRLLRKKGKDMWGVRLFDEADTAAARRTNRVA |
| | KRRRNREKARIGYLKELFATEIEKIDSGFYQRLDDSKFWEEDKTVQQKFALFADTGY |
| | TDVDYYKEYPTIFHLRKKLLESTEPQDVRLVYLAILNIYKHRGHFLNANLDENGNDD |
| | FADIYANFVKMAKDVVDIDFSQDISDNWLRETLSSSELSKTGKHSAIIEHYGIKKSAE |
| | KKKVEMLKLICGLKGVLSKIFEDTEWGDEDKKFSISYRDSNYDETSQKAEELLSESA |
| | YELFCTMKQMHDWSLLANIMKGEDGEYKYLSLARVAAYEKHQKDLKILKKLYKDN |
| | DLKKYNEMFRIMNKNNYSGYVGSVNSDKDKTRRGAQCSRDDLFKKIKEDIKDFAET |
| | AEKNYVLEEIGKENFLPKQLTASNGVIPNQIHKKELKKILENAENYLPFLREKDESNL |
| | TVSERIVKMFEFQIPYYIGPISPIVNGDMKYSKNVWSVRKEAGKVYPWNFEQKIDTEE |
| | SAQRFISRMVKHCTYIGGEEVLPKNSLLYEKFMVLNELNNLRINGERISVKLKQDIYE |
| | YLLKKEKKVTKSKLIEFLRIHGEVEPQATPEISGIDDGFTNKRANYCKFCRLFGVDTL |
| | TYEQEKIAEQIISWSTVYGDTKKFVKKKIQDNYGDVLSDKQIAQAAGYKFKDWGRL |
| | SEELLKVEGIDKSTGEESTVIQKMWSDNYNLMELIESDNFTYKQNIEERAKVIDKTLS |
| | EIVYEDLDELYISAPVRRMTWQTILVLKEITEVMGEAPARVFVEMARDPNAEKKRTE |
| | SRKNKFLALYKNCKKEERELYNQINKRDEADFRSKKLYLYYTQKGRCMYSGERIDL |
| | DDLFNDNLYDIDHIYPRHFVKDDSIDNNLVLVKKEINAHKSDGYPLADGIQKKCRVF |
| | WNELKSGGFITAEKYERLTRTFEFTEDERVSFINRQLVETRQGTKAITDLFKKTFPQA |
| | DIVYVKAPNVSAFRQKYKLLKCRNVNDFHHAQDAYLNIAVGNVYHVKFTKNPYNFI |
| | REYKKAPEKNPYHMDKIFNYPVVRNDEVAWQTKNDESITMVKSMMKKNTPLVTRR |
| | NFEVHGALENKLGIKSAQLAKKANGVGYIGVKTSDARLQNVCRYGGVTDIKGAYFC |
| | LVEHTQKKKRIRTIEVVPLYLKHQLDTKEKLEQYFAESYGYENPNVRIEKIKMYSLIK |
| | VNGFYMYLTGRSKKRLLVCNAVQMVLTQEWMMYIKEITKAYNAQYSDEYMQKQK |
| | LISKLKNEKLYDVLCEKHLHSIYRLRPNGVGQKLLEGRQKFEEISLLEQIYVILQILQL |
| | SQLINNGANLDIIEKGAHAGVATLNKMISECNEFKLINQSITGLYENEVDLLTI (SEQ |
| | ID NO: 62) |
| 2186264 | MEKIKKYNIGLDIGTNSVGWAVVESGQQKIIKKGGKALWGVRLFDAASTAKDRRLK |
| | RSIRRRYDRRRQRIKLLQDEFANEINKVDSNFYKKLQDSFISPNDKNNSKTELTDFDK |
| | FNIFSNNIRNKINNISEEKKYPTIYHLRNDLVNSKDKKDIRLVYLAIHHIIKYRGNFLYE |
| | NSDFNVNNINLKEKIEETFNHYIEECMPGEFNIPINYEKIANILLKDSKNEIKDLLKDEL |
| | LVIDTYNKGFSSEFAKMIVGNKFKIERLIPVSNEEKSMSISFNGNDFDEEYEKLEELLG |
| | QNIEILDELKELYDNLFLKRLFKGSESNNISSLMISIYDEHKKDLKMLKKIFKNNKKIY |
| | NELFKGENCKYEEYIHNKITQEDFCKIILNSYEKLGMSEETIEFYNKNLKSKIENNSLL |
| | PRISEPSNGKYPFQLNRNELIKIIDNQKEYYKFLGEKINNKYKLVKLLEFKIPYYVGPL |
| | VSSDKSCFAWMERNNNEKITPYNFDEVVNKELTAEKFIKRMISHCTYLINEPALPSNS |
| | LLYNKFKVLNELKQIKVNDRKMTLEQQQNAIKDLFEKTSGSITEKKFINYLKQTKEW |
| | DMYSEINVKGYSADKKFANNMQSYVDFFGKNGIFDNTNYNEENAEEIIEWITIFNDK |
| | EILESKVRDTYPDLTDDQVTKILRKKYSGWGELSKKLLTLPYYKDKETEVYKSIIDLM |
| | FETKENFMQILNNDEYKFQNMIKENNKITNTKELNYKDTVSELATSPAVKRGIFQSLK |
| | IIEEIVDYMGYEPDSVSIEMSRGDDKKERKPDRKKYLTELYKKSKDSIENYNTLNKQL |
| | VEKEIDSQKLFLYFIQEGKCMYCGKSLNVEDMISYEIDHIIPRTLIKDDSIDNKVLVHK |
| | ECNQNKAASFVLPPYYRSDYNKKWWTRLKNIGLVSNKKYNNLVREKYSEDDINGFI |
| | NRQLVETRQIIKHVANIIENYYDTKVIYLKTNLSHNYRERYELFKFRELNDYHHAHD |
| | AYLAAVLGEYKEKYMKYNITYDLVRTLNNKIKEKGQTEKLKYGFIINSLDSDVSSVI |
| | DGLNKNKINKETGEMIFSPDAFNKTIENNLYRNDILISYKTEYNTGEFYNQTIYGKGK |
| | GNIRLKDNLPTEIYGGYSGVNVSYFMLVEYKGKRKIIGIPILIAKNNNEKDLNDYIINQ |
| | INFSEYRVLKNKIPYNTLFEYKGQQVYIKGYSSKNKNGEISNAIQLKISKELYLKWKY |
| | ALNRILNNKENNLNESEYKENLIEILNYLLQLNDYYPLFDNEIIQIKKYVEKNYQNLGI |
| | KDFEKIIKQLLILYKCSSENANLKEFGLKDRIGRLTGKVISEGTIIYKSITGIREKKYEF |
| | (SEQ ID NO: 63) |
| 2186290 | MNNNYVLGLDIGITSVGYGVIDIENGEFVDYGVRLFEEGTPAENKKRRDSRSRRRLT |
| | ARRHNRIHDMQKLLKENGIMLDSYHPFQNIYELRCKGLTKELTKDELTAVILNITKH |
| | RGSVIETVEEDASKSEDELSLKATLQQNEQLLKNGKFVCQIQLDRLNNKNSVRGHEN |
| | NFSTKDYLRELEVILQHQNLNDDLINKILDIVGRRRAYYEGPGSEKSPTPYGRFVEVD |
| | GQIQYFDMIEKMRGKCSVFPEELRAPKMSIRADLFNILNDFNNLSVNGNKLTVEEKE |
| | RLLKIISKKGKISLGELAKELDVDKNSIKGFRVDKKDKPIFTEFKGYKKIKSILEKEKPI |
| | SLEDCEMIDSVIEILTNKKGIQERKEALQHLEYHFSNAVVETLANTTGITGYHSLSFKA |
| | LRLLNKELFESEMNQMQLLHELKLFDKNRVSYKGKKRIEANSDAILSPVAKRAQNET |
| | FKVVNALRKKYGEFDSIVVEMTRDKNSKEKQKRIKAFQSMREEGSQNMNKMLEEW |
| | GYDPSEVNGKTRMKIRLYEQQNGKSAYTLQPLDLNRVIKDPSYTEIDHIIPISISLDDS |
| | QNNKVLALRSENQMKGNLTPFLAYSSNKFQGMGCSYSEYKTNVLSNKNIPYRKRLN |
| | LLNEEDIIKEDIAKKFINRNLVDTSYACRVVLNTLSNYFKDNEIDTKVHTINGRVTDIF |
| | RKKIHLEKQRDEDYLHHAIDALIVASIKKMNLLNGYLAKQDHDFNDLYNEETGEIRT |
| | IPGDKVFFDAKYIQYIINLKTIYQESSQYYNHVIDKEAMNFNPIKISYKIDTKPNRQLFD |
| | EQIKGVRTFDNKKYVIEKISDIYNPKENTKLIEDILNNNCEKYFINKNDPRTFEVIKNIV |
| | KEDYEIYQSNKEIYKVQNGKIKYVNKYEKDVDTKKVKIENPLKKFYEENGAITKFSN |
| | KKMGPPVKSLKYKGKLLKEHAEAIDHTQNRTIGVKLKLKPYRTDFYKCKDGKVRFV |
| | TIRYMDVEYKKNDNMYHINQETYEELLTRKKIDSHAIFLFSLHTNELLMIERDEGTNF |
| | YFNPNKVSSLKYDGKNFELFMFLGTNNDSMNRIEVKPRYKFTKDRINPITTTFLQCK |
| | KMATDVLGNLYEMKDNKLKLDFK (SEQ ID NO: 64) |
| 2186416 | MGRLVLGLDIGITSVGYGVIDIDKYEFVDYGVRLFKEGTAADNEIRRNTRGHRRLISR |
| | KATRINDMKHLLKSLGIINEDFHSLDNVYEIRVKGLKEKLSNDELATAILHITKRRGST |
| | IETVDDTEEASKETGELKEILQANSKKLADGKFICEVQLELLNNGESIRGHHNNFKTK |
| | DYIKELNEILSHQELPEEACEAIVQIVSRKRAYYEGPGSQKSPTPYGRYIEANQKEPID |
| | LIEKMRGKCSVYPDEPRAPKLAVSSDIFNLLNDLNNLTYDGNRKITMDQKDEVFKIV |
| | DKKAKVEPKDIAKILGTDLSNISGFRIDKKKNPLKSEFKGYAKLKKCFKECGETISLK |
| | DYEALDQIIEILTNRKGIEERKQLLNLVNSSLITDETVNKLASLDGIANYHSLSLKAMR |
| | ELNEEMYVSNYNQMQLIQQIHKGKDVSALKGKKSIEADDTAILSPVTKRAQRETFKV |
| | INALRRKYGEFDTIVVEMTREKNSADQKKSINNRQKFYEEKNKEVDTLLKENGYNP |
| | DKINGKTKMKVRLYLEQDGKSAYTREPLSLGEVITNPRYTEIDHIIPISISLDDSYNNKI |
| | LATANENKAKGNRTPIDAYNKHLFDNPTLGLSGDLQTYISVVQSSKRFSRRKKNYLL |
| | YKDDITKMDVVQKFIARNLIDTSYANRVVLNTLTDYFKANEIPTKVFTLNGNITHKFR |
| | TQINLPKDREEDYLHHAVDGLIIASVKTMGLLNGCLAKYELKDLYDKDTGEVKKVP |
| | GKEEFFDPKYIEFISTLKNIYDESNKYYRGIMTRDRLAYAPIKISHKVDTKPNRQVADE |
| | TIYSTRVVDGQEILVQKYKDIYDPKFKNLVEDIVNGKAEDKYIMAKKDPATFEEIVRI |
| | VLNHYETFKDDPKQYVKDKKGVKLKGENPLTAYKEEFGKIRKFSKKGNGPEITCIKY |
| | YAEKLGNHIDISKNYDTKHGKKVILKQISPYRTDFYRDTDGKVKMITIRYKDVSFKES |
| | LGLYCIDRDWYQTEKTKKKISDEAVFLCSLHHDELVGIVKKEGQKYIYDDSTESDGN |
| | TKYHDGVTPEVVKFTATNNDIKNIVEFKPRYTKSKKQLMVAIGTFVDVKKYATDVL |
| | GNLYEVKDNNLKLEFK (SEQ ID NO: 65) |
| 2186474 | MENKKNKKIKEYNIGLDIGTNSVGWAVTDLDNNILKHNSKNMWGARLFEEGKTAA |
| | ETRSFRGARRRIERRRERINILQSLMLDDVEKEYPNFFPMLRETSKIKEEKNAEAINGK |
| | KYNLFSELNFSDATYYKEYPTIYHLREDLIKTQKKLDIRLVYLAIHHIIKYRGNFLYEG |
| | DLKSSNDKILDSVNILVSFIQENLDINFNSKAENICRILMNKEKTKVEKKDELLKLFDY |
| | DKSEKSIISNIISAILGYKFDVNKIFESNLDNSSISFSNEIANEEEIKEAIKEQCEIYEILQRI |
| | YSWFVLQDILQGNTNISTAFINKYNKYKKDLKLLKKVYKDNLKKEYNKMFRIEDNN |
| | SYAVYDKNISKCSRENLYKRIKQDLDGIDGCKEKEEILKYIENDEFLVKINTTENAAIP |
| | YQLHYQELEKILENQSRYYLTLKENKQHILELMKFRIPYYIGPLAKSGDGKFSWIVRN |
| | SDEKILPWTFEKIVDTDKTAEKFIKRMTNKCTYLLNEDVIPKQSILYSEFCVLNELANI |
| | KVNNHKLAPKEKEMIIEKLFKYRKTVKLKEFKQFLIDYQIYKEVDSIIGLAEEDRFISN |
| | MSSYIEMKEILGNIDEKNIEKVENIIEWITIFEDKKILKRKIKKEYHLDDNTIEKLAKKN |
| | YNGWSRLSKKLLTEMKSYDDNKSVMEKLKSTRENFMQIINNTKYGFDKQIEEKMRE |
| | KQKDNITYEEVNEIPTSPANKKAIWQCLKIVKEITKIMKREPKNIYIEFAREEQEKKKR |
| | NDTRAKQLLKIYEKYEEQIQILRDYNPKIYKELKNKQNDKDFNERLYLYFTQNGRCM |
| | YSSKPLNIDTLHLYEVDHILPQSYVKDDSLSNKVLVYKKENQRKSDSLLLNEEIINRQ |
| | QTWWKQLRKSGLIDDKKYNNLIRRKMFETNYDKVTFISRQLVETRQSTKYIANLLLA |
| | QYKNTDVFAIRSQLTHRFREYFEIYKNRNINDYHHAQDAYIISVIGNVIDTKFYFRNE |
| | YKYAEYVKSYIKKEEEEKDKKNKPWIIMKMISNNINHDEVKRTLNYKDCYVTRKLE |
| | EQTGAFYNQTLYSPKDKNALIPLKENMQAEKYGGYSNEYKAYFVIYSYIDNKGKKQ |
| | VEMIGVPVKIAYDIKNKRITLVDYITTIVKNISEIKIIKGKILKYQEYINKENEPMVLLS |
| | DSEVRTNKQLIVVPMINRLIYIMNKEKITEEEKEEIQKEMQNIYDYLLEKLKNEYKVY |
| | GSIYQKLNDKKTRKVYENLTYEDKKIIINGLIDLMHKGQGNLKKLNQGDRAGRKTG |
| | KSFKTKELSEMTFVDKSITGMYERRYRVNGMEDHCSN (SEQ ID NO: 66) |
| 2186478 | MKKEYNIGLDIGTTSVGWAVVEVDNQKVMKKGKKALWGARLFEEADTAADRRTS |
| | RSTRRRYDRRRERIKLLQEEFKDEINKVDQEFFTKLQNSKYSKKDKNNKRLRYSEEEI |
| | NGAAAYYKVYPTIYHLRKALMEEQKQFDIRLVYLAIHHIIKYRGNFLYEGKNFSVKD |
| | ISLEDQLRKLIELLIKELPELELTEDDAMDIDFKEFAEAFFKETRNDVKVTLKSILKSID |
| | STFASEFSKLMVGNKANIPKMLKQDSSEKIEVTFLKSDYENKYDKCLTALGEKIEIYD |
| | LIKKIYDSVTLKKILGKEDNPSISNKMIKMYDKFSKDFRWLKETLRKDREVYNAFFKS |
| | SKGEKSPYEKYMSSDIDNNELKNSIDKSISALIEHNNISQENLDKYKNEILPQLENNTF |
| | LCKTNSTDNGIYPYQLNENELIQIIENQGKYYPFLLEKYNEDTYRIVRLLEFKIPYYIGP |
| | LVTKEKSKNAWLIKTTKTGKITPFNFDTMIDKEKTAIEFIKRMSSHCSYLLDEYAMPT |
| | NSILYSEFKVRNELKQIKINEKSIDLNLQQKIFEEFFKTTTGSITERKFINYIKTTGEFSM |
| | YNDDIKITGFSSTNSFANNMQTYIDFFGTNGIFENTKYNIADAEQIIEWITIFEDKGILE |
| | KTVKDNYPLLSNEQIQLILNKKYKGWGNLSKKLLTTKYYKDKQTEVYKSIMDLLKE |
| | TKENFMQIINNKEYKFQKMIEEYNNNSNATLSKLEIVNKLATSPANKKGIYQALKVV |
| | DEIVGYLGYEPKNIMIEMSREEGKKVRTVDRKSKLQNLYNECKNQIVDYKRLRHEL |
| | DSRNITTEKLYLYFIQEGKCLYSGKPLEIEKIEDVSMYEVDHIIPRSLVKDDSIDNKAL |
| | VIRKYNQEKKDNLVLPKAYRNLAQIQWWQRLKNNGLMSAKKFHNLTRDEYSDEDI |
| | DGFINRQLVETRQIIKHVANILGNEYRKTKILYPKASLSHNYRERYELYKFRNINDYH |
| | HAHDAYLAAVLGEYSDKYMKKSFDFSQIKEINKKIIELDEKEKIKYGYVINSLDERVS |
| | NIVINSSKNMTDEKTGEVLFDAKKFNETVAKNLYRNDILISRKTEIRSGQFYKKTIYK |
| | RCVGTIPIKKDMPVDIYGGYSNNETKYLKLVKYGNGKKKLIGIPMNLAALNDTKIID |
| | NYIKYQLKLKNSDTYEVLKNKIPFETEIIYNNQNVYIKGYSVMGRTCEVSNAHELKIK |
| | KDLLYNWRYALNYFINGKEKDVEEAKKHSIEIYNFLVESIDYPLFNNELKKVRENIKF |
| | DTLEIEKQKKVILELFKLFKCSSTNANLKEFGLSDRIGRLSGINIKDGYIITKSTTGLKE |
| | FKYKI (SEQ ID NO: 67) |
| 2186573 | MEGITMDNKYVLGLDIGISSCGYGIIDLETGKFVDYGVRLFKEGTAEDNGKRRDARS |
| | RRRLTSRRHTRIMDMQKLLKENGIMSNDYHPLQNVYELRCKGLSEKLTNDELTAAIL |
| | HITKHRGSVIETVEEDAKKSEDELSLKATLQHNEQLIKQGKYICQIQLDNLNNRNHIR |
| | GHENNFSTKDYVNELNEILHHQELDEQFINKIIDIVARRRAYYEGPGSEKSPTPYGRFI |
| | EVDGQIKEIDLIEKMRGKCSVFPDELRAPKMAVTADLFNFLNDMNNLTVDGNKLCP |
| | EEKKGILTIVSQKGNITLRQLAKELNVDEVDIKGYRIDKNQKAIFTEFKGYKKIKSILE |
| | KEGYSISLNDYEMLDRIIEILTNKKGIQERKDYLYHLEYRLSDSVVDTLANTTGITGY |
| | HSLSFKALKLLNKELFESEMNQMQLLHELKLFDKNRVSHKGKKNIESDPEAILSPVA |
| | KRAQNETFKVVNALRKKYGEFDSIVVEMTRDKNSDEKKKRISNYQKIRESGSKEMD |
| | KMLIEKGYDPTKINGKTRMKIRLYEQQGGKSAYTFEPLDLNRVIKDSKYTEIDHIIPISI |
| | SLDDSQNNKVLALHSENQVKGNLTPFMAYAANKFNGMGCSYSEFKKNVLSNDNIP |
| | YKKKLNLLNEENITKEEVAKKFINRNLVDTSYACRVVLNTLSDYFKDNEINTKVHTI |
| | NGRVTDLFRKKIHLEKDRDEDYLHHAVDALIVASVKKMNLLNGYLAKQEHDFNDL |
| | YNEETGEIITIPDDKVFYDERYIQYIVNLKTLYQESNQYYNRMIDKEDMHFNPIKISHK |
| | IDTKPNRQLFNEQIKGFRKIEGKNYVIEKISDIYNSKENIKLIEDILNGNCEKYFIAKND |
| | PRTFDIIKNIVLADFEIYKDNKDIYKIDKNKIKYVNAYKADKETKEIRIKNPLTKFFEEN |
| | GPVTKFSPKRKGPPIKSMKYKGKILNEHVEAVDRTQNKLLGARLTLKPYRTDFYRCE |
| | DGKIRFVTVRHVDVEYKKSDGMYHIDNGLYNELLNKKKIGSQAEFLFSLHTDELLYI |
| | KKEIGESFCFSPKEKFVYKDSDYELFTFLGTNNDVRNVIEVKPRYMFTKTRINPYTTT |
| | FVECKKYATDVLGNLYEVKDNKLKLEFK (SEQ ID NO: 68) |
| 2186655 | MKYNYILGLDVGISSVGWGLLALDENNNPYKIIDVGSRIFPPGEVEKTGDSRAKERRE |
| | KRGARRVTRRREFRIDRVRNLLFEEGYLKGNITKSKVSEKNEELTIIYNDMINSYYKN |
| | KNTNPYKLKVEALDRKLTNDELCIILVHYAKKRGYKSNREESADKENGKVLSAIKEN |
| | EEIIKTKHYRSISEMYIKDEKFKDKIKNSAGNYKISVTNEMYLDEINKVLDKQITFGLI |
| | TNEFKQKYLEIYNSRRHYSEGPGNYYTYDESGKRITNRSKYGGNLIEKMIGKCAFDK |
| | KPRAPKYAPSSELFVAISKLVNLRYKQENEQYTKLSSEEIKQIIAEAKQKNKITYKTLI |
| | KILNKEKIKIKDLNLSKKEYAKVIEELKNQLKISKDEKLDIYSLEDSKREIYEKIYNDK |
| | LYSKALIELKGYHTLRKVITDCYDKSIWTEVSNNIEFLDELALYCTNYKLNDEILKSIK |
| | ESQIIEEKFEDYNFVEKLPNFKDHLMLSTEIIRKIIPLMEEGNTYDKAMAKIGYDHSNL |
| | YDKIEKKDLLVPVCVDRNITNQRVIRSLTQTRKVINAIIKKYGTPKIVNVETARELAKS |
| | RRERNEISKNQEQKQEENEQDKNHLVELGFFSDINKISSNDLLKYRLWKEQKEFCGY |
| | SMEKIKIEDLFRNNIVQIDHILPYSRTYNDNYLNKTLVYTKYNQDKGNKTPYEWFGK |
| | TSKWEEYESYINNLSISQKKKDNYLLKKLDFETEREMREQNLNDTKYISKELATLLKS |
| | HLNVEEVNTYPGAITSKLRARWGFNRLTHSYISKTYIMPENMKKDINKDRDNHLHH |
| | AMDALVIASITKSLQQKITLYEKFSRYIDGKLKEKISDITINDEAITYDNETGEVISTNY |
| | KEYLKEQLSKDNIFYNKHNIGKLKFPLPYENFVQEAEARVYEQDINTLRYELQNLRT |
| | YTKKELKNVHILTPSLAKTKATGAMHKETYYGIYEEKDKDETIRTWKTLRTPIEKVK |
| | RKDLENIQGKDGGSKDIYNALVEWFGKDETGEKALKRHEGKYPINPNDKEQKEIKK |
| | VKIYTEYKNTGHMIKDANVEKGGIYRIEIYKSKDEKDNKLYFAAYDMYEIKTIERIKK |
| | STKENKPVPALRVKVDYGRDKNTMIIPYEKLTTDYKLYKVLNKSDLIKVILKNGKES |
| | MGYVVGFTSGMLEIKSIIGDGYDLISEGNIFTQFRTQYTITVSTIKEIEKLSISILGEISGL |
| | (SEQ ID NO: 69) |
| 2186742 | MKKEKYVLGLDIGISSVGWAAVRLDENDNPIRIIDANSKIFSPGEVPKTGASKNLIRRT |
| | KRDTGRVIRRRSFRIDRVRHLLNLNGYLGDDIIEGLVSDVNDKLTLIYSNMLNKYYK |
| | NKNTNPYELKVAALDRKLTPDELSIILVHYAKHRGYRSNREDNTDDKETGKVKKSIT |
| | DNEELMQNKNYRIISEMFLKDEKFKDRIRNSTDDYKMCVSREMYQKEIEIVLDSQIK |
| | YGLITEEFKNDYIKIWSSQRHYAKGPGKPSKYGGDLIVRMTGKCKFDNNLRAPKYAP |
| | SSEIFVALTNLVNLRYHKFGNINYIGLTKEEIEKVIALAKSKDNVTYASLFKELGYTN |
| | VTIKGLSLSKKEYNKALTKFKSKYKIDEKSTIDFNTLSGEKKIEFNELKTKEVLSRNFIS |
| | LKAYSDMRKAFSTKLNTEEWYKISDNYELLDTIATILTNYKLNEDVEKAIKENNIDER |
| | YKDIIKSLPNYKEHMMLSLDLIRKLNKIMITGKTYDTAMEELGYNHSLVNENSVKED |
| | LLIPINQDNELTNQRVIRSLSQARSIINSVIKKYGMPERINIETARELAKSMEERREIEN |
| | KQKENHENNEKTREEIFNLGIFKSKDDIKSFDLLKYKLWKEQLEHCTYSGEPITIEDLF |
| | DNNIVQVDHILPYSRTFDDTYFNKTLVLSKYNQDKKNNTPYEWFGKDKEKWNNFV |
| | GYISSLNIPTKKKDNYLLTNLTPEMEAEYRNQNLNDTKYIARYLVSYIKANLNVKKV |
| | GCPSGSITGKLRNYWQLNGLTHSLESETYYVKNRDENTKKNRENHLHHAMDAILIAI |
| | TNEKIIKRVSDYERYKRYLDNKPISYIINYAKSMDDSFEINNFTDEETGELYENSLKKY |
| | IADLRTNEFFQTNKKNNTIKTYLPTPYADFREEAIVRVYEQNKEILRNKLRAFHYNEE |
| | DLENAVPLIPKFAKNKVGGPLHGETYYGLKTYKDGNETKYYKTSRISLNSDKFTKDK |
| | LDNIIDKDSGSRIVYDTISAWLENDNGKEAFAKHNGYPINPTTNAPIKKIKIKSDYDGK |
| | GHIIGNKIVEKEDIYQIDIYKKKDEDRLYFVAYDAMELYQIKERKDKNGNIIKPLYNIP |
| | VDLWWGQGKNHKKMKYLDLQENYEFYVSLQKNEFIEIILNNGNKGYAYVKGFSSG |
| | QFEVGSALGDSLDIIGEKNLFQGARERYQLTISTIKSIKKVKLNNLGKIE (SEQ ID NO: |
| | 70) |
| 2186752 | MKVHFGLDIGIASVGWAVVNENYEILEAASNIFSSADASKNVERRNFRQSKRLLRRR |
| | RTRILDFNKLWCANGMTIPETRCNNQLELRVLGLKEELSQEEIYYVLSNMLTHRGISY |
| | LEDAIDDSNAGKSDYEKGLIYNQKELDTKYPCEVQFERLQKYGKYRGHIVVNDNNG |
| | EKITLSNIFTISAYEREIVDFFNEQRCYHNFLTDDFEEEYLDIFRRKREYYEGPGNEKSR |
| | TDYGKYTTKINPENGEYLTEDNIFEKLIGKCSVYPQFRRAAAASYTAQEFNVLNDLN |
| | NLIINGRKLKQEEKEEIIRELLSAPARIDMRKIIKKVIGEDIESFSGARIDKDEKEIFHTFE |
| | VYRKMKKAFMDKDLSIDRFEMNQLDLIGEILTINTDKKSIVEAFKREQEKIQATDEEI |
| | DIFVEFRKKNNQLFSKWHAFSIQIMDELIPEMYNQPKNQMELLTEMGVFRNKGKAFA |
| | EMTSLTPNVVTEEIYNPVVKRSVRVAVDIINALVKKYGYPEEIVIEMPRDNNSEEEQK |
| | RIKDTQKKNEKELADIISKVKKEYGRDITGADFREHKKLALKLKLWNEQQERCLYSG |
| | KVISINDLLDNPNLFEIDHIIPRSISFDNSRSNKVLVYATENQKKGNFTPYMYLSAVNR |
| | EWNYEKYKAYVLSLKASKSIGSGKINNLLYTEDITKVQVLKGFISRNINDTRYASRTI |
| | LNVLQAYFKEKNPDTKIKVVRGGFTHQMRENLKLDKDREKSYAHHAVDAMLICYS |
| | QMGYEAYHKLQSQFIDFEEERIIDKNAWQQQMNDRVYEDTLYQNKWQQIKSNIVEA |
| | EKHVKYWHKVDKKANRGLCNQTIRGTRVSEGEVYKINKINIYTKDGIKTFRNMIEGG |
| | KEKSFLMYENDPKTFENLMIIYNQYKDTDNPFAAYEKETGDFVRKYSKNHNGPRIEC |
| | IKYRDGIVNSCIDISHKYGYEKGSKKVILESLKPYRMDVYYNKEKQQYYFIGIKYSDC |
| | KFEKGGYTISEEAYTKALISEKIIKEGQSRKDLDSLGWEYQFSFYENDIIEYEKDGIIYT |
| | ERFLSRTMPQARNYIETKPIDAPKFEKQNLVGLSKTTSVRKLYMDILGNWHYCRGEK |
| | FKLHIDNV (SEQ ID NO: 71) |
| 2186833 | MMEFKVALDIGVASVGWAIVQNDLKVVDSGARLFDSADPSKNVERRDNRGTRRLL |
| | RRKAHRLTVFDRFWVGKGLDSMDEITENSLILRNKAIIERIDIKEVYCVLRNMLKHRG |
| | ISYLDDAIDDNVKGAYKEALNNNALELKVKFPCEIQLERFNEFGKFRGDVIIELDKKE |
| | QRALSNVFTKSGYRREIEQFFEVQGAYYPFMDETFITEYMDIFESKRDYYVGPGNEK |
| | SRTDYGRFTTKIDTETGKYITEDNIFEKLIGKCSVYNDELRASSASFTAQEFNVLNDLN |
| | NLTVNGRELEEDEKRKIIKSIFTAKSMRMRKFITEAIGEPIIDMKGARIDGKDKEIFHTF |
| | EAYREIKKALNTIDVDINDLSLESMNLSAHILTINTEKDAIIKAFAKTDMHLANEVVE |
| | CFIKTRKKTPQLFNKWHSLSLKIMNELIDELYKRPVNQMVLLTEMGLFKDNIEKFKG |
| | LLKIPVDSVLEELYNPVVRKAVRQSIEVLNAIMKTYGQPTDIIIEMARDKNEEDQKKR |
| | IKKIQGDNEKELAKIIKKISLEYGVDIEQKHYHQNKGLKTKLRLWNEQGGVCLYSGK |
| | PIVIADLLYDSEKFEIDHVIPISISFDDSRKNKVLVYRSENQTKGNRTPYMYLSTVSRE |
| | WDFERYAYHISELEKKKLITTYKKRNLLFKENITKQDVVKGFIQRNLNDTRYASRVV |
| | LNCVQDYMRANDKPTKVKVIRGSFTSQLRKRLNLPKDRDESYAHHAVDAMIMCYS |
| | LMGLDAYKSSQKDVIDYETGEILDQKVYDKLSEEELYNRYMFYMRLMEIKLAIKKG |
| | EERIKYSHKVDKKVNRKLSNETIYGVREKIDGTKQKISKINNIYDINGYVNFKNKKDK |
| | NKLNDFLMYHHDPKTFEKLLKILEEYADQPNPFLTYKQEIGEPIRKYSKKDNGPFINS |
| | LKYYDGEVGSCIDISHKYGHEKGSKKVVLDSLSPYRTDVYYREEDKSYHLVGIKYA |
| | NFKFMKGEYILDEESYNEILVSEKLLKSGECYTDIEKLGYEFRFSLYKNDIILYEKNGE |
| | EKIERFLSRTMPKVKNYIETKPIDANKFSKQNPVGLGKTTNVVKINTDILGNKHFSQK |
| | EKFIIKFKLDR (SEQ ID NO: 72) |
| 2186873 | VKERGNYFLGLDLGTSSIGWAVTDQNYRLIRAKGKDLWGVRLFKEAETSESRRTFR |
| | VSRRRRQREVARIGLLKEFFSEEISKVDPSFYQRLNESKYHRDEKLVDSRHALFSDKS |
| | FTDQDYFSCYPTIFHLRKELILSKEPHDVRLVFLAILNLFKRRGHFLNTNLDAEQSLGF |
| | NTLFDKLLYLLSDRFDLRLDTSIVIQNIEETLSDKTYSRTQKAEQIAAELSVKKTSDRM |
| | FYELIRAITGLKFTIDVMFGDKYRDSDNNKMSLSFRDNNYDEEVINISEIIGEENMEIIN |
| | ILKLIHDQALLSNVMQGHKYLSEARVESYQKHHEDLKLLKKTIKKFTIEDYDILFRSD |
| | EDGSYSAYVGSVNSRKKQRRNMKKRNSEEFYVNLKKILKKCPSTDADVKYILKEIK |
| | NENLLPKQLTSDNGVIPNQVHLAELKAILCNASKYLNFLNEKDEKGLTISDKIIGLFSF |
| | RIPYYLGPLNNYHSNKGGNAWIVRKSKGQVLPWNIEEMVDEEETSKVFIERLVRKCT |
| | YIKDETVLPKSSLLYERFLVLNELNNLKIRGVKPDVELKQEIFNELFLAGKKVSRKKL |
| | NSFLVSRGHINPNEIEAITGIDGDFANSLSTFGKFHSVLGDLACEDEGKAIIEQIVFWGT |
| | VYGDDKKFYKEKLKRELADKLNAEQIKKIIGFKFRDWGRLSKEFLEMQGVNKSTGE |
| | MLPMIQMLWNYNENIMELLSDRYTYTETLINEYSAISKTLSELEYDDLEGLNLSSPVK |
| | RMVWQTLLLVKEIEKTLGNPPERIFVEMARERGERGKRTTSRKQRLIDLYKNCKKDT |
| | RDWVGELENLEDSILRSKKLYLYYVQNGRCMYTGEPIDLDKLMNDNLYDIDHIYPR |
| | RYVKDDSLDNNLVLVKKQTNASKSDQYPIDNSIYIKQHAYWKSLLTKDMTDGFITRE |
| | KYNRLINRNPLSEEQLAGFINRQIVETRQGTKVITQILKQAFSGSEMVYVKARNISDFR |
| | DEFDLIKVRSVNDFHHAQDAYLSCVVGNTYYVKFTKDSLNFVKESRRNNNSKYHMS |
| | KIFHYTVKRGEEVAWIAEEQSGDNSIRIVKTMTNKNTPIITKMTLEGHGKLFDATLYS |
| | SQKAKGESYVPLKTTDSRIQNVQKYGGYTSVSTAHFFLVEHVEKGKRIRTIEALPLMF |
| | RDSIGTNLTKLNDYCKNVLQLKEPDVRLAKIKLQSLIKRDGFYMNITGKTLNRNIVSN |
| | AVPLILSRKWLDYIKKVEKTVERDYTDHKITSELNMRLYEELTEKHKNSIYSKRPNPI |
| | GKVLKTGQEYFAKLDLMEQCFVLSQILQISQLSNMGANLKLIQGSKKSGISLIPKNIS |
| | MAEEFLLVNQSITGLYCSEIDLKTI (SEQ ID NO: 73) |
| 2186985 | LGIRFGLDIGIASVGWAVVNDNYEVEEAGSNIFSSADANQNTERRMFRQTKRLHRRR |
| | KNRISDFKKLWIDTGLEIPDLNINNILELRIKGLKEKLSQEEIFHVLRNELLHRGISYLD |
| | DAIDSSAINQSDYAKGINENQKELDNGCFPCEIQAKRLKKYGKYHGNTSVGTEEDKII |
| | LSNVFTTGAYKKEIQAILEQQQQFSSFLTESFCGKYMEIFLRKREYYVGPGNEKSRTD |
| | YGIFTTKIDKTTGKYFTDKNIFEKLIGKCSVYKNKLRAAGATYTAQEFNLLNDLNNISI |
| | NGRKLSKQEKEAVIEEVKTANSFNMRKILKKITGEEIETLTGARLDKNEKEDFHKFAQ |
| | YNTMRKEFEKNGWDITTLSREELDNIGNILTLNTEKEAIIKEFIHKDLSLTEEMTACLV |
| | NLRKQKSSLFNKWQSFSLEIMLELIPGLYEQPKNQMELLTDMGAFKSNKDLFTEYKN |
| | IPAEIILENIYNPVVSRSIRVTIQIINALIKKYGYPDQIIIEMPRDKNEDEMKKRISDCQK |
| | NNQKELDNIIKRISSEYGIKITDQDFYQHKKLVLKLKLWNEQGGKCLYSGKKIDIHDL |
| | LYNPTLFEIDHIIPKSISFDDSRSNKVLVYSTENQKKGNTTPYIYLNSIKKEWGYNEYM |
| | AYVLQLKKDKLITKNKMEKLLFTEDITKIQVLKGFISRNINDTRYASRVVLNTLQNYF |
| | KAKNAITKIKVVRGSFTHQMRDNLHLDKNREESYSHHAVDAMIMCYSQMGFDNYH |
| | KLQTDLIDVETGEIIDIHKWQNEINEKTYNELIYQNKMIHIRANIQKAEKQIKFWHKR |
| | DTKPNRGLCNQTIRGTRTENGVIKKVNKLNIYNPSDVETLKKMIKNGKEDSFLMYQH |
| | DPRTWENMIQIMQDYRSSKNPFADYENETGGYFRKYSKKHNGPRITKLKYLNGEVN |
| | SCIDISHKYGLETGSKKVILESLKPYRMDVYYNEKEQKYYLIGIKYSDLKFSKNKCIIN |
| | EEAYKKILVYEKLITPEQNRADLEKLGYKFMLSFYKDEIIQYEKNGEYFTERFLSRTM |
| | PKQRNYIETKPIDAPQFEKQPRHLVGLGKTTSIKKIRLDILGNRYYCTQEKFQTEC |
| | (SEQ ID NO: 74) |
| 2187023 | VKENEEYNIGLDIGTASVGWCVTDLNNNVLKIKGKNMWGSRIFEEAQTAAERRNFR |
| | SARRRLTRRKERINILQDLMREDIEKEYSNFLPRLKETSLKYEDKIFSNSKYNLFSDNN |
| | RHEISYYEEFPTIYHLRNKLIKEENKVDIRLVYLAIHHIIKYRGNFLYDGDFSNDTSKV |
| | DDNLEIILEFLNQNYQIELKSKDDLKNILLSKNLSKAQKKDELMACFNFVSSDKTIITN |
| | IINAILGYSFEINKIFDCDLEKSKIKFSNEIDNEEEIENSLESNIDIYYALKDLYSWYILQD |
| | IMSNCNYISESFMKRYEKFKEDLKLLKKIYKEYFPNQYDEMFKVKGKNNFVNYIGKN |
| | KGEKFGKCDPEDFFKMLKSKIKELPDTCTYKEQIELDLQNDDFLRKLNIIDNGAIPYQ |
| | LHKNELSIIIKNQSKYYNTLKENGEKIISLLEFRIPYYVGPLANDKFNKWNWVVRNNE |
| | NEKIRPWNFSEIINEEETAEKFINRMTNKCTYLLNEKVMPKESLLYSKFCVLNELNNIR |
| | INEKHIPRDLKKKIVDKLFKQNKKVTIDMFKELCKIEGLPECDIIGIQDGKNFMSNMSS |
| | YMDMSKIFGKVDENNYDMCETLIYWITIFEDKKILKKKIKATYSTITEEQLNKLLKLK |
| | YSGWSRLSKKLLVGLKSNDYETIMDKLEKTSMNFMQIINNKNFGFDKKINDLAPVID |
| | KNITYKDVSEIPTSPANKRGIWQAICVVKEITKVMNAEPKNIFIEFARSEEFNKNMSQK |
| | RAKKLLKIYDNIQDELATLKEYDSNVYEKLKENKSNKDFSERMQLYFMQLGKCLYS |
| | GEPLNIEDLNLYEVDHILPQAYIKDDSIDNKALVKRIENQTKKDNLLLSDKIINNMSG |
| | YWKKLLDLGLITKEKYLRLTKRKIFENDYEQENFVKRQLVETRQITKYVTNLLKNSY |
| | TNTDIFTIRAELTHAFREEFGIYKNRNVNNYHHAHDAYIISIIGNVMNTYIRNNEEFKY |
| | GDFVKKYLKENKNKAEKNGMIIGSIKKNIDIGNIKKVFYYKDCFISRMLIEGTGAFYD |
| | QTLYSPKINKNKLISLKDDKDSTKYGGYSGEKKAYFSILEYINENGLKEYKLLGIPIKV |
| | AYDIKAKKYSEEEYIKKVFFKNNDITELKVIKKKVLKNQQYLDENKEVMRFCSDSEI |
| | RVDKELVLNYKMNELIYLMNMKENQLKDNEKDLLNNNFEYMFDYLLEKIKTEYKV |
| | FSSIYKKLLDKKEQYMLLENEDKKCVINGLVDLMKNGQGNLKKVGLTEREGRKSKQ |
| | NFDTKRLTNMIFIDKSVTGIYERRINIYGMENSSSK (SEQ ID NO: 75) |
| 2187103 | MSFCQIFIKVGGKTMKKKYYIGLDMGTSSCGWAVTDENYCLVRAKGKDMWGVRLF |
| | SEAQTAAERRTHRIARRRLQREKARIGYLREYFSNEINKVDPGFYQRMDDSKYYPED |
| | KREEQKNTIFADRDYTDQDYYKEYPTIFHLRKELIDSEEKHDIRLIYLAILNMFKHRG |
| | HFLNANLGDGEVGNLNELYQSLTEMLSEFMEISLVSLDSTKELEHILVSKEYSNTAK |
| | WEQLMEVLNVKKNKNKSEAEMLRLICGLSGRLSKIFVNDAFDEESAKLSISFRDGNF |
| | EDKCVTVEQILSEESYEIFLLLKQIHDWGVLAGIMKGYEYLSYARVESYEKHKKDLEI |
| | LKRVFQKYDKKQYDEMFRCMKKDNYSAYVGSVNSGKESARRTEKTTATDFYKNLE |
| | KRVKKLPVDEDTSYILDEISKETFLPKQLTPSNGVIPNQVHKKELIKILKNAEKHYVFL |
| | SRKDETGLSVSEKIVQLFEFQIPYYIGPFDNADGKGNGWAVRKENGPVMPWNIEEKI |
| | DTKQSAEKFIEKMVNHCTYLNGETVLPKNSLLYEKFMVLNELNNLKINGESISVDLK |
| | QQIYTQLFQKGKKVTAKKLINFLKIHNYVDQEDVEVSGIDGDFVNTLSNHKKFCEVF |
| | QVDCLSQSQLEMAEKIIFWSTIYGDSKKFLQEKILEEYGEVLDAAQIKRITGYKFKDW |
| | GRLSKQFLQLEGADTDTGEVQTIISRMWNENYNLMELLSNHFTYLDEIENKSAKIEK |
| | TLQDIMYEDLDELYISAPVKRMVWQTILILKEIYQVMGCAPERIFVEMARDQDAPKK |
| | RTISRKNKFAELYKKCKEDGRDWLKEIKNIDESRFRSKKLYLYYTQKGRCMYTGEPI |
| | DLEDLFNDNLYDIDHIYPRHFTKDDSIENNLVLVKKEKNAHKSDSFPIENSIRMKQHG |
| | WWKTLQDGGFITKEKYKRLVRNTEFTNEELANFVSRQIVETRQGTKVITQLFEKTFP |
| | ESDIVYAKAGNVSAFRQKFDLLKCRIVNDFHHANDAYLNIVVGNTYFVKFTKNPYQ |
| | FINGYRKDPSKYRFHMYKIFDYLVRNRNEIAWQTENDQSIRIVKKMMRKNTPLITRM |
| | NYEEHGGIADQTIYSAADAKKAKGVGYLPIKASDNKLANTEKYGGYKKFTGTYFFL |
| | VEHTVKKKRIRTLEAMPLYKVENLDTIEKLENYCRQELKLDDPSIRMKKIKMYSLIK |
| | VNGFYLYLTGRSGNQLLVSNAVPMVLSYDYVKYVKKISQIQERDHSLEEIEEMEEISS |
| | KKNLELYNQLTYKYSKAIYAQRPNPIGDKLSNWKEKFISLNIKDQINVLLQILQLSML |
| | SNMGADLTAIGGVKKTGVATLNKKISDKDEFKLINQSVTGLYESEIDLLKV (SEQ ID |
| | NO: 76) |
| 2187334 | MAKRYNIGLDIGTSSVGWAVVEADTQKIIKKGNKKTALWGVRLFDGAQTAESRRQS |
| | RSTRRRYDRRRQRIQLLQEEFKNEINKVDVNFYTKLKESKYNKSDKINKTIIISKDEQK |
| | LINEYNKKYPTIYHLRNKLVISDEKEDIRLVYLAMHHIIKYRGNFNYQGDHFNVNDL |
| | NIKDKLIFLLTSLSEQYPELEIDEEYSSKITDDIIQNSLLNPSKNDMKINTKELLLELTSN |
| | SKFVSEFIKLLNGNKFSIKKLFGIEELEEDIQISFSNSDFDDNYEKLEQTVPDKIETLNIM |
| | KELYDMIFLKKLFVGSDSTNLSALMVERYNQHKEDLKFLKSIFNIDRKIYNKIFRDGK |
| | EKCLYTKYITNEISNDEFIKEIEKYLPKKYEGSNEKIRMDNGIFLPRITSTENGKYPYQL |
| | NEDELIKIIEKQGKYYKFLLDKVDDKYKIVKLLEFKIPYYVGPLNKNSQFAWLEKTIN |
| | EKITPYNFDKIVDKESTAEKFIKRMISHCTYLDDKVDNNGNIIEENYALPNCSILYSEF |
| | KVRNELKQIKINGDSIKNENQHKIFDELFKKTNGTITEKKFKDYLKVCPDYYPFEGNF |
| | NITGYSADGRFANNMQSYVDFFGENGIFDGTDYTLDDAEKIIEWITIFEDKDILKSKV |
| | LNNYDKLSEEAVKKILNKKYSGWGSLSRRLLCGIVTKDKKSGIGKTIMDYMMETKE |
| | NFMQILNNDEYDFQGKISEYNKVNNHVKLNYSLVENLATSPSTKRGIYQALKVVDEL |
| | TKYIGYAPENIMVEMAREDEKKVRKDDRKTYLEKLYKENKIEIDNYNRLYSELESRD |
| | KIDSEKLYLYFIQEGKCLYTGEPINIDELNNDEYEVDHIIPRTLIKDDSIDNKALVLRRC |
| | NQDKKDSFVLPKEYRTERMLDWWHHLKKMKLMTAKKFYSLKRKEYTDDDIQGFIN |
| | RQLVETRQITKHVADILGSLYSKTKIVYLKANISHNYRERYELFKFREINDYHHAHDA |
| | YLAAVLGEYKEKYMKCKIDFESVKELNKKFYENGDYKKLKYGFVINCLDENEFATIS |
| | KIIKNFVDDKTGEVLFDVSKFNRTIENTLYRNDILISKKTEIRTGEFYNQTKKKKCNDG |
| | IKLKGNMPTAMYGAYTSVNPSYAVMVEYDYKKCKNRKMVGMPIYLKTDAEKVSY |
| | LKMKLGLNEGDSLDIISKPIPFYAYLNWDNQLCYLVGASDKVEVVNAKEFNYNKEF |
| | YIKHKYTLHKVFHNKNYEIEDSKYSQELSEIIKYIVDKIENEYELFKNLVPELKSIVKY |
| | NEKIDFSVENKEKIIIQLTKLLNCTSDNANFKFLNDSYSSAFGKKHGRTIEHCLFVSKS |
| | VTSIYEATNEF (SEQ ID NO: 77) |
| 2178264 | MKDDNEKAKFILGIDVGISSVGWAIVDLDELDSPYKIKDLGVRIFTAAEVPKTGDSKA |
| | KSRREKRGIRRLIRRKAFRMDRVRYLLNQNGFLGEKITDGKVDIINHNLEKEAAKIIE |
| | NYNIGKNIMPYELKVKGLTEKLLKEELLIIMLHYAKHRGYKSNREDNEAAKESTSES |
| | GKTLSAINDNRKIMIENKYEIVSELFLNDDKFKGRIRNTNNDYKMCVTREMYLEEIN |
| | KVLDKQISLEVITSEFKDEFIKIWSDQRHYSKGPGGNSKYGGDLIERMVGKCQFTGEP |
| | RAPKCAPSSEVFVSVSKLVNLRYKQPDDKEYKSLTENEITIAIELAKEQDKVTYKKLI |
| | KELKLPDNTIIKGLELSKKDFIEFIVKFKKEVLNREDKEKVDKATLTEEESNEYDKRY |
| | FDKLNSKTCLELKSYSIIRKAFIKQLGTEKWDNIKNDFDLLDEFARLLTYYKLNEEIKR |
| | ELDKIENLRDEYKDVVISLPTFKDHLSISLSLVKSLIPLISSGMRYDEAMNELGYNHSD |
| | LNGDIEKYDLLVSLNKGTSINNQRVMRSLAQTRKVVNAVIKKYGIPYQINVETAREL |
| | AKSRQERNEIEKKQRENQENNEKIKNDLVDKFPNIFSDTSKVSGVDIQKYKLWIEQNE |
| | TCAYSLERITIEKLFDKNEVQIDHILPYSRTMNDNYSNRTLVLTKANQEKGNRTPYE |
| | WFGKTDKWANFENFINNINISTSKKDNYLLINLTQEVEQEMRDQNLNDTKYISRHLS |
| | TMIRKYLNVEKFQMVSGSITGRLRARWGLNNLTHSLVSPTYYQSGKNKDREHHLHH |
| | AMDALVIAVTTPSLIQKVSHYEKFRRYIVGKTETEIRNATSDEVFTFYETGEVLTFRE |
| | YLNKTIEEKYLFQNKKGTMDVLFPLPYDKFITEAKLRVYERNEEVLKRELLELKTYT |
| | REEIDNATVIIPSFMKIKPSGAYHAETFYGIKKVEDTTYKIEKKKVTSLKKKDLEDIVE |
| | KNGGSKEVYNTLVDWFEDNEEGSEVFKQKGYPVNKKSGNLIKAVRLQEEYKGKGHI |
| | INGAVVEKDNIYQIEIYKKDGSDKLYFIGLDNLEINKIKNGKEFNVTLWYGRDNNKID |
| | NISYKKVNEEYTKVASLKKNDLIEIELNSGVKGKCCANGFSGGKFEISSLIGDGYDLV |
| | SSKLFGNLIERYQIAISTIKSIEILKISVLGKIE (SEQ ID NO: 78) |
| 2178932- | MGLRFGIDIGVASVGWAVVNDEYEVVESGANLFPSADASKNVDRRTFRQMKRVHR |
| H596A | RRKTRVSDFDKLWKNHGRKLPESNPEEVLELRVKGLTQALTEEELYAVLKNALLHR |
| | GISYLDDAGDEAVSGRSDYERGIMLNRQALDENMHPCEIQFKRLKEYGSYRGNLTID |
| | VDGEKVMLSNVFTTGAYRKEIFEILERQQESHAFLTDDFVENYLKIFSRKREYYVGPG |
| | NEKSRTDYGKYTTRIDAETGQYVTEDNIFEKLIGKCSVYKDLTRAAGATYTAQEFNL |
| | LNDLNNLTVNGRKLEKEEKVQIIEEVKTANTVNMRKILKRVIGEELETLTGARIDKKE |
| | KELFHHFEQYNAIRKAFEKQGWDISELSREDLDSIGDILTLNTERESILGGFQREGLTL |
| | SEEKKDCLIALRKKSGALFSKWQSFSLKIMQELIPELYAQPKNQMQLLTDMGVFKTN |
| | QEKFAEYHQIPDEMILENIYNPVVSRSVRITVKVLNALVKKYGNPSQIVIEMPRDNSE |
| | EEEKKRTKEIQAKNEKELEGIVKRISEEYGIKITEQDFHQHKKLVLKLKLWQEQDGIC |
| | VYSGRAISIQDLLKNPYLFEIDAIIPKSISFDDSRTNKVLVYRTENQEKRNQTPYLYLK |
| | NLRRSWGYDEYRSYVLELKRKGLIPKAKLDKLLFTEDITKIEVLKGFLSRNLNDTRY |
| | ASRMVLNILQSYFKAKKADTTIRVVRGSFTHQLRNVMRLEKDREESYAHHAVDAML |
| | MCYSQMGFDAYHARQKEIIDFETGEILDENKWNQIMSDKAYDELMYQNQWMRIRA |
| | NIQKAEKEVKYWHKRDSKPNRGLCNQTIRGTREKDGATMKVNKLNIYEPSGWAKL |
| | KKMIEQGKQERFLMYLHDRKTWDDMLRIIEEYQDAANPFVEYEKETGDYLRKYAK |
| | KHNGPRIRQLKYLDGEVGSCIDISHKYGHEQGSRKVILESLNPYRMDVYYHAGKKA |
| | YFFVGVKYADLKFEKGRYILNEEAYQRALVSEKLIAASQNRFDLEELGYRFVLSFYR |
| | NEIIEYEKDGEYYTERFLSRTMPKQKNYIETKPMDAPKFEFVDRHFVGLRKTKSVKKI |
| | RLDILGNRFYCSQEKFTLKVDVV (SEQ ID NO: 499) |

TABLE 1.1 lists exemplary NLS sequences.

TABLE 1.1
Exemplary Nuclear Localization
Signal Sequences

| SEQ | | |
|---|---|---|
| ID | Descrip- | |
| NO: | tion | Sequence |
| 433 | NLS | KRPAATKKAGQAKKKK |
| 434 | NLS | PKKKRKV |
| 435 | NLS | LPPLERLTL |
| 500 | NLS | KR(K/R)R |
| 501 | NLS | (P/R)XXKR(\hat{ }D/E)(K/R) |
| 502 | NLS | KRX(W/F/Y)XXAF |
| 503 | NLS | (R/P)XXKR(K/R)(\hat{ }D/E) |
| 504 | NLS | LGKR(K/R)(W/F/Y) |
| 505 | NLS | KRX₁₀K(K/R)(K/R) |
| 506 | NLS | K(K/R)RK |
| 507 | NLS | KRX₁₁K(K/R)(K/R) |
| 508 | NLS | KRX₁₂K(K/R)(K/R) |
| 509 | NLS | KRX₁₀K(K/R)X(K/R) |
| 510 | NLS | KRX₁₁K(K/R)X(K/R) |
| 511 | NLS | KRX₁₂K(K/R)X(K/R) |
| 512 | NLS | APKKKRKVGIHGVPAA |

*wherein X is any naturally occurring amino acid; and \hat{ }D/E is any naturally occurring amino acid except Asp or Glu

TABLE 2 provides consensus PAM sequences.

TABLE 2
Consensus PAM sequences

| Consensus | |
|---|---|
| PAM sequence | SEQ ID NO |
| NNNAAT | SEQ ID NO: 79 |
| NNGNMA | SEQ ID NO: 80 |
| RNGKNK | SEQ ID NO: 81 |
| NNNAWWT | SEQ ID NO: 82 |
| NNG | SEQ ID NO: 83 |
| NNGRM | SEQ ID NO: 84 |
| WNRRAW | SEQ ID NO: 85 |
| NNGRA | SEQ ID NO: 86 |
| HNYAR | SEQ ID NO: 87 |
| NNGKT | SEQ ID NO: 88 |
| NGG | SEQ ID NO: 89 |
| NGGR | SEQ ID NO: 90 |
| NGGNR | SEQ ID NO: 91 |
| NNNMAWT | SEQ ID NO: 92 |
| NRNACYN | SEQ ID NO: 93 |
| NNNAHAA | SEQ ID NO: 94 |
| NNNWWWT | SEQ ID NO: 95 |
| NRNAAY | SEQ ID NO: 96 |
| NNRACY | SEQ ID NO: 97 |
| NNNGMWM | SEQ ID NO: 98 |
| NRNAWC | SEQ ID NO: 99 |
| NNGNNNN | SEQ ID NO: 100 |
| NNAARY | SEQ ID NO: 101 |
| NNNAAWT | SEQ ID NO: 102 |
| HNRNWW | SEQ ID NO: 103 |
| HNYAAR | SEQ ID NO: 104 |
| NGGWY | SEQ ID NO: 105 |
| WNAWMWW | SEQ ID NO: 106 |
| WNGR | SEQ ID NO: 107 |
| NNRAAA | SEQ ID NO: 108 |
| WNWRRM | SEQ ID NO: 109 |
| NNYGMNY | SEQ ID NO: 110 |
| WRG | SEQ ID NO: 111 |
| NNNGHWW | SEQ ID NO: 112 |
| NAAAAW | SEQ ID NO: 113 |
| WGGNR | SEQ ID NO: 114 |
| HNNAA | SEQ ID NO: 115 |
| WNGRW | SEQ ID NO: 116 |
| NNNRANK | SEQ ID NO: 117 |
| WNWWHWW | SEQ ID NO: 118 |
| NNGTAW | SEQ ID NO: 119 |
| NNAMRWA | SEQ ID NO: 120 |
| NYAAR | SEQ ID NO: 121 |
| NNKRR | SEQ ID NO: 122 |

TABLE 3 provides ambiguity codes.

TABLE 3
Nucleotide Ambiguity Codes

| Code | Represents |
|---|---|
| A | Adenine |
| G | Guanine |
| C | Cyosine |
| T | Thymineridine |
| Y | Pyrimidine (C or T) |
| R | Purine (A or G) |
| W | weak (A or T) |
| K | keto (T or G) |
| M | amino (C or A) |
| V | A, C, G (not T) |
| H | A, C, T (not G) |
| N | any base |

TABLE 4 provides exemplary repeat sequences.

TABLE 4
Exemplary Repeat Sequences

| Effector Protein(s) | RNA sequence |
|---|---|
| 2178307 (SEQ ID NO: 1), | GUUUGAGAGACUUGUA (SEQ ID NO: 123) |
| 2181509 (SEQ ID NO: 23) | |
| 2178521 (SEQ ID NO: 2) | AUUUAAGUACCUGGAGA (SEQ ID NO: 124) |
| 2178891 (SEQ ID NO: 5), | GUUUAAGAUCUAUGUCA (SEQ ID NO: 125) |
| 2179042 (SEQ ID NO: 10), | |
| 2179107 (SEQ ID NO: 14), | |
| 2179109 (SEQ ID NO: 15), | |
| 2181365 (SEQ ID NO: 21), | |
| 2186264 (SEQ ID NO: 63) | |
| 2179018 (SEQ ID NO: 9) | GUUUGAGAGUCUUGUU (SEQ ID NO: 126) |
| 2179044 (SEQ ID NO: 11) | GUUUUAGCUCUAUGUUA (SEQ ID NO: 127) |
| 2180100 (SEQ ID NO: 16) | GUUUAGUUACCAUAUG (SEQ ID NO: 128) |
| 2180100 (SEQ ID NO: 16) | GUUUUGUUACCAUAUGG (SEQ ID NO: 129) |
| 2180475 (SEQ ID NO: 17) | GUUUUAGAAGUAUGUUA (SEQ ID NO: 130) |
| 2180475 (SEQ ID NO: 17) | GUUUAAGAAGUAUGUUA (SEQ ID NO: 131) |
| 2181249 (SEQ ID NO: 20) | GUUUUAGAAUUAUGUUA (SEQ ID NO: 132) |
| 2181249 (SEQ ID NO: 20) | GUUUAAGAAUUAUGUUA (SEQ ID NO: 133) |
| 2181445 (SEQ ID NO: 22) | GUUUGAGAGUAUUGUA (SEQ ID NO: 134) |
| 2182747 (SEQ ID NO: 29) | AUUUAAGUACCUAGUAG (SEQ ID NO: 135) |
| 2183116 (SEQ ID NO: 31), | GUUUGAGAGCCCUGUA (SEQ ID NO: 136) |
| 2183288 (SEQ ID NO: 37), | |
| 2183638 (SEQ ID NO: 43), | |
| 2183868 (SEQ ID NO: 44) | |
| 2183137 (SEQ ID NO: 32), | GUUUGAGAACCUUGUA (SEQ ID NO: 137) |
| 2183156 (SEQ ID NO: 33), | |
| 2184029 (SEQ ID NO: 45), | |
| 2185882 (SEQ ID NO: 54), | |
| 2186215 (SEQ ID NO: 62), | |

2186873 (SEQ ID NO: 73),

| | |
|---|---|
| 2187103 (SEQ ID NO: 76) | |
| 2183168 (SEQ ID NO: 34) | GUUUAAGUACCUAGUAA (SEQ ID NO: 138) |
| 2183262 (SEQ ID NO: 36) | GUUUGAGAACCCUGUA (SEQ ID NO: 139) |
| 2183304 (SEQ ID NO: 38) | GUUUGAGAGACUUGUU (SEQ ID NO: 140) |
| 2183526 (SEQ ID NO: 40) | GUUUGAGAGCCUUGUU (SEQ ID NO: 141) |
| 2183548 (SEQ ID NO: 41) | GUUUGAGAGCUUUGUU (SEQ ID NO: 142) |
| 2184261 (SEQ ID NO: 46) | AUUUAAGUACCCGGUGA (SEQ ID NO: 143) |
| 2184558 (SEQ ID NO: 47) | GUUUAGUUACCAUAAGG (SEQ ID NO: 144) |
| 2184558 (SEQ ID NO: 47) | GUUUUGUUACCAUAAGG (SEQ ID NO: 145) |
| 2185070 (SEQ ID NO: 49) | GUUUUAGUAUUGUGUUA (SEQ ID NO: 146) |
| 2178932 (SEQ ID NO: 6), | AUUUAAGUACCUGUAAG (SEQ ID NO: 147) |
| 2185440 (SEQ ID NO: 51), | |
| 2185986 (SEQ ID NO: 56) | |
| 2185499 (SEQ ID NO: 53) | GUUUUAGAAUUGUGUUA (SEQ ID NO: 148) |
| 2185499 (SEQ ID NO: 53) | GUUUAAGAAUUGUGUUA (SEQ ID NO: 149) |
| 2185938 (SEQ ID NO: 55) | GUUUAAGUUCUAUGUCA (SEQ ID NO: 150) |
| 2186029 (SEQ ID NO: 57) | GUUUAGCUACCAUCUAU (SEQ ID NO: 151) |
| 2186035 (SEQ ID NO: 58) | GUUUAAGUAUUGUGUUA (SEQ ID NO: 152) |
| 2186149 (SEQ ID NO: 59) | GUUUUAGAAAUAUGUUA (SEQ ID NO: 153) |
| 2186149 (SEQ ID NO: 59) | GUUUAAGAAAUAUGUUA (SEQ ID NO: 154) |
| 2186167 (SEQ ID NO: 60) | GUUUAAGUACCUGGUAG (SEQ ID NO: 155) |
| 2186416 (SEQ ID NO: 65) | GUUUAGCUACCAUAUGG (SEQ ID NO: 156) |
| 2186474 (SEQ ID NO: 66) | GUUUUAGAGUUGUGUUA (SEQ ID NO: 157) |
| 2186573 (SEQ ID NO: 68) | GUUUAGUUACCAUAAAG (SEQ ID NO: 158) |
| 2186752 (SEQ ID NO: 71) | AUUUAAGUACCUGUAGA (SEQ ID NO: 159) |
| 2186752 (SEQ ID NO: 71) | AUUUUAGUACCUGAAGA (SEQ ID NO: 160) |
| 2187023 (SEQ ID NO: 75) | GUUUUAGAUUUAUGUUA (SEQ ID NO: 161) |
| 2187023 (SEQ ID NO: 75) | GUUUAAGAUUUAUGUUA (SEQ ID NO: 162) |
| 2178958 (SEQ ID NO: 7) | AUUUAAGUACCUGUAAA (SEQ ID NO: 163) |
| 2181613 (SEQ ID NO: 25) | GUUUUAUUACCAUUAAU (SEQ ID NO: 164) |
| 2186290 (SEQ ID NO: 64) | GUUUAGUUACCAUAUGA (SEQ ID NO: 165) |

TABLE 5 provides exemplary sgRNA sequences.

TABLE 5
Exemplary sgRNA Sequences

| Effector | |
|---|---|
| protein | sgRNA Sequence without spacer sequence |
| 2178307 (SEQ | GUUUGAGAGACUUGUAGAAAUACAAGACGAGUGCAAAUAAGGAUUUA |
| ID NO: 1) | UCCGAAAUCGUCGCAAGACUCUACAUGGUGUAGAACUUAAAAGACUUG |
| | GUUUCAAG (SEQ ID NO: 166) |
| 2178521 (SEQ | AUUUAAGUACCUGGAGAGAAAUUAUCUUCAGACCUACUUAAACAAGGC |
| ID NO: 2) | UUUAUGCCGAAAG (SEQ ID NO: 167) |
| 2178891 (SEQ | GUUUAAGAUCUAUGUCAGAAAUGACAUAGAGAGUUUAAAUAAAGCUU |
| ID NO: 5) | UAAGCCUAAAUGCUCACGUAUGUGUUCGAACAGUUA (SEQ ID NO: 168) |
| 2179018 (SEQ | GUUUGAGAGUCUUGUUGAAAAACAAGACGAGUGCAAAUAAGGUUUAA |
| ID NO: 9) | CCGUAAUCGUCGAAAGACUCUGCAUGGUGCAGAAAGAAAGAGGCUGUA |
| | UAAAGCCU (SEQ ID NO: 169) |
| 2179042 (SEQ | GUUUAAGAUCUAUGUCAGAAAUGACAUAGAGAGUUUAAAUAAAGCUU |
| ID NO: 10) | UAAGCCUAAAUGCUCACAUAUGUGUUCAGACGGUUA (SEQ ID NO: 170) |
| 2179044 (SEQ | GUUUUAGCUCUAUGUUAGAAAUGACAUAGAUAAGUUAAAAUAAAAGU |
| ID NO: 11) | UUCCUAUAUGGAUACUCUAAACACUUACUAUUGUAGGUUGCUGCGUGU |
| | GCAGCUCUUU (SEQ ID NO: 171) |
| 2179107 (SEQ | GUUUAAGAUCUAUGUCAGAAAUGACAUAGAAAAGUUUAAAUAAAGGC |
| ID NO: 14) | UUAGCCCUAAAUACUCCUUUGGAGAACGACUGUGUA (SEQ ID NO: 172) |
| 2179109 (SEQ | GUUUAAGAUCUAUGUCAGAAAUGACAUAGAGAGUUUAAAUAAAGGUU |
| ID NO: 15) | UUACCCUAAAUACUCUUCUGAGUUGCUGCUUAGGCA (SEQ ID NO: 173) |
| 2180100 (SEQ | GUUUAGUUACCAUAUGGGAAACUAUAUGACCUAACUAAACAAGGGUUU |
| ID NO: 16) | AUCCCGGAUUCGGCUCUUCGGAGCC (SEQ ID NO: 174) |
| 2180100 (SEQ | GUUUUGUUACCAUAUGGGAAACUAUAUGACCUAACAAAACAAGGGUUU |
| ID NO: 16) | AUCCCGGAUUCGGCUCUUCGGAGCC (SEQ ID NO: 175) |
| 2180475 (SEQ | GUUUUAGAAGUAUGUUAGAAAUAACAUACCGAGUUAAAAUAAGGUUU |
| ID NO: 17) | AACCUUAAAUACUGGCUAUGCCAGAUUCAUUUAGAU (SEQ ID NO: 176) |
| 2180475 (SEQ | GUUUAAGAAGUAUGUUAGAAAUAACAUACCGAGUUUAAAUAAGGUUU |
| ID NO: 17) | AACCUUAAAUACUGGCUAUGCCAGAUUCAUUUAGAUGAAGUAGCGCCU |
| | CUUUGGAGGUGCUUUA (SEQ ID NO: 177) |
| 2181249 (SEQ | GUUUUAGAAUUAUGUUAGAAAUAGCAUAAUGAGUUAAAAUAAGGUUA |
| ID NO: 20) | AACCUUUAAUGCUGAUUUAUAUCAGUUUCGCUUAGG (SEQ ID NO: 178) |
| 2181249 (SEQ | GUUUAAGAAUUAUGUUAGAAAUAGCAUAAUGAGUUUAAAUAAGGUUA |
| ID NO: 20) | AACCUUUAAUGCUGAUUUAUAUCAGUUUCGCUUAGGUGAAAUGAGCUC |
| | UUUGAGCUCUUUUU (SEQ ID NO: 179) |
| 2181365 (SEQ | GUUUAAGAUCUAUGUCAGAAAUGACAUAGAGAGUUUAAAUAAAGCUU |
| ID NO: 21) | UAAGCCUAAAUGCUCACUUGUGUUCGGACGGUUAGC (SEQ ID NO: 180) |
| 2181445 (SEQ | GUUUGAGAGUAUUGUAGAAAUACAAGACGAGUGCAAAUAAGGUUUAU |
| ID NO: 22) | CCGAAAUCGUCGUAUGACUCUACAUGGUGUAGAUAAUAAAAAGGACUU |
| | GCGUGCAA (SEQ ID NO: 181) |
| 2181509 (SEQ | GUUUGAGAGACUUGUAGAAAUACAAGACGAGUGCAAAUAAGGAUUUA |
| ID NO: 23) | UCCGAAAUCGUCGCAAGACUCUACAUGGUGUAGAACUUAAAAGGCUUG |
| | GUUUCAAG (SEQ ID NO: 182) |
| 2182747 (SEQ | AUUUAAGUACCUAGUAGGAAAUAUUAGAUCUACUUAAACAAGGCUUUA |
| ID NO: 29) | UGCCGAAAUCAAGGACACCGACGGGUGUCC (SEQ ID NO: 183) |
| 2183116 (SEQ | GUUUGAGAGCCCUGUAGAAAUACAGGGUGAGUUCAAAUAAGGCUUUAG |
| ID NO: 31) | CCGAAAUCGCGCCUCGUGUCCGCAUUGUGCGGGUUUAAAAGAGAAUCG |
| | UAAGAUU (SEQ ID NO: 184) |
| 2183137 (SEQ | GUUUGAGAACCUUGUAGAAAUACAAGGUGAGUUCAAAUAAGGAGCAA |
| ID NO: 32) | UCCGAAAUUACAUUGACCGCAUUGUGCGGAUAUGAAAGACGUAGCCUA |
| | UGGCUAAG (SEQ ID NO: 185) |
| 2183156 (SEQ | GUUUGAGAACCUUGUAGAAAACAAGGUGAGUUCAAAUAAGGAUUUAU |
| ID NO: 33) | CCGAAAUUACAUUUGCCCGCAUUGUGCGGAUUGAAAAGGUUCGACUUA |
| | UGUCGAAU (SEQ ID NO: 186) |
| 2183168 (SEQ | GUUUAAGUACCUAGUAAGAAAUUAUUAGACCUACUUAAAUAAGGCUUU |
| ID NO: 34) | AUGCCGAAAUCAAGAACACCCGACGGGGUGUUC (SEQ ID NO: 187) |
| 2183262 (SEQ | GUUUGAGAACCCUGUAGAAAUACAGGGUGAGUUCAAAUAAGGCAUUAG |
| ID NO: 36) | CCGAAAUUGCACCUGAUGUCCGCAUUGUGCGGGAUUAAAGAGGAUCGA |
| | AGGAUUC (SEQ ID NO: 188) |
| 2183288 (SEQ | GUUUGAGAGCCCUGUAGAAAUACAGGGUGAGUUCAAAUAAGGCUUUAG |
| ID NO: 37) | CCGAUAUCGCACCUCGUGUCCGCAUUGUGCGGGAUUUUAAGAGAAUCG |
| | UUUCGAU (SEQ ID NO: 189) |
| 2183304 (SEQ | GUUUGAGAGACUUGUUGAAAAACAGGUCGAGUGCAAAUAAGGUUUAU |
| ID NO: 38) | CCGAACUCGUCAAUUUGAUCCGCAUUGUGCGGUUUUUAAAAUCCGAUG |
| | UUUCAUCG (SEQ ID NO: 190) |
| 2183304 (SEQ | GUUUGAGAGACUUGUUGAAAAACAGGUCGAGUGCAAAUAAGGUUUAU |
| ID NO: 38) | CCGAACUCGUCAAUUUGAUCCGCAUUGUGCGGUUAUUAAAAUCCGAUG |
| | UUUCAUCG (SEQ ID NO: 191) |
| 2183526 (SEQ | GUUUGAGAGCCUUGUUGAAAAACAAGGCGAGUUCAAAUAAGGAUUUU |
| ID NO: 40) | UCCGAAAUCGCUAUUUAGUCUGCAGUGUGCAGUUUCGUGUCCGGCUUU |
| | GCCGGGCA (SEQ ID NO: 192) |
| 2183526 (SEQ | GUUUGAGAGCCUUGUUGAAAAACAAGGCGAGUUCAAAUAAGGAUUAU |
| ID NO: 40) | UCCGAAAUCGCUAUUUAGUCUGCAGUGUGCAGUUUCGUGUCCGGCUUU |
| | GCCGGGCA (SEQ ID NO: 193) |
| 2183548 (SEQ | GUUUGAGAGCUUUGUUGAAAAACAAAGCGAGUGCAAAUAAGGUUUAU |
| ID NO: 41) | CCGAAAUCGCUUUUAUAGCAAGCAUUGUGCUUAUUAAAUCUGGCUUUC |
| | CAGCCAGA (SEQ ID NO: 194) |
| 2183638 (SEQ | GUUUGAGAGCCCUGUAGAAAUACAGGGUGAGUUCAAAUAAGGCUUUAG |
| ID NO: 43) | CCGAAAUCGCACCUCGUGUCCGCAUUGUGCGGGUUUAAAGAGAAUCGU |
| | UUGAUUC (SEQ ID NO: 195) |
| 2183868 (SEQ | GUUUGAGAGCCCUGUAGAAAUACAGGGUGAGUUCAAAUAAGGCUUUAG |
| ID NO: 44) | CCGAAAUCGCACCUGAGUGUCCGCAUUGUGCGGGAUUAAGAGAAUCGA |
| | AAGAUUC (SEQ ID NO: 196) |
| 2184029 (SEQ | GUUUGAGAACCUUGUAGAAAUACAAGGUGAGUUCAAAUAAGGAUUUA |
| ID NO: 45) | UCCGACAUCGCCGUAAGGCCCACAUUGUGUGGUUUAAAAGACUUGAGA |
| | AAGUCUUU (SEQ ID NO: 197) |
| 2184261 (SEQ | AUUUAAGUACCCGGUGAGAAAAUUUCUUCGGACCUAUUUAAACAAGGC |
| ID NO: 46) | UUUAUGCCGAAAG (SEQ ID NO: 198) |
| 2184558 (SEQ | GUUUAGUUACCAUAAGGGAAAUUUUAUGACCUAACUAAACAAGGGUUU |
| ID NO: 47) | AUCCCGGAUUCGGCUCUUCGGAGCC (SEQ ID NO: 199) |
| 2184558 (SEQ | GUUUUGUUACCAUAAGGGAAAUUUUAUGACCUAACAAAACAAGGGUUU |
| ID NO: 47) | AUCCCGGAUUCGGCUCUUCGGAGCC (SEQ ID NO: 200) |
| 2185070 (SEQ | GUUUUAGUAUUGUGUUAGAAAUAGCACAAUGAGUUAAAAUAAGGUUU |
| ID NO: 49) | AAACCUUAAAUGCCAACUUGAUGUUGGUUUCACUUA (SEQ ID NO: 201) |
| 2185440 (SEQ | AUUUAAGUACCUGUAAGGAAAUUUACAGAUCUACUUAAAUAAGGCUUU |
| ID NO: 51) | AUGCCGAAAUCAAGGAGUGCCGACGGGCACUCC (SEQ ID NO: 202) |
| 2185499 (SEQ | GUUUUAGAAUUGUGUUAGAAAUAGCACAAUGAGUUAAAAUAAGGGUA |
| ID NO: 53) | ACCUUAAAUGCCAAUUCAUAUUGGUUUCACUUAGGU (SEQ ID NO: 203) |
| 2185499 (SEQ | GUUUAAGAAUUGUGUUAGAAAUAGCACAAUGAGUUUAAAUAAGGGUA |
| ID NO: 53) | ACCUUAAAUGCCAAUUCAUAUUGGUUUCACUUAGGUGAAUAAAGAGUU |
| | CUAGCUUGCUAGGGCUUUU (SEQ ID NO: 204) |
| 2185882 (SEQ | GUUUGAGAACCUUGUAGAAAUACAAGGUGAGUGCAAAUAAGGAUUUA |
| ID NO: 54) | UCCGACAUCGCCGUUUGGUCUGCAUGGUGCAGGUAAAAGACUUGGUUU |
| | CAAGUCUU (SEQ ID NO: 205) |
| 2185938 (SEQ | GUUUAAGUUCUAUGUCAGAAAUGACAUAGAGAGUUUAAAUAAAGGUU |
| ID NO: 55) | UUACCCUAAAUGCUCACGUAUGUGUUCGGCUGCUUA (SEQ ID NO: 206) |
| 2185986 (SEQ | AUUUAAGUACCUGUAAGGAAAUUUACAGAUCUACUUAAAUAAGGCUUU |
| ID NO: 56) | AUGCCGAGAUUAAGGAGUGCCGACGGGUACUCC (SEQ ID NO: 207) |
| 2186029 (SEQ | GUUUAGCUACCAUCUAUGAAAGAUAUAUGAUCUAGCUAAACAAGGGUU |
| ID NO: 57) | UAUCCCGGAUUCGGCUCCUAUAAGGAGCC (SEQ ID NO: 208) |
| 2186035 (SEQ | GUUUAAGUAUUGUGUUAGAAAUAGCACAAUGAGUUUAAAUAAGGUUU |
| ID NO: 58) | AACCUUAAAUGCCAAUUUAUGUUGGUUUCACUUAGGUGAAUUUAAGUU |
| | CUAGUUAUCUAGAGCUUUU (SEQ ID NO: 209) |
| 2186149 (SEQ | GUUUUAGAAAUAUGUUAGAAAUAGCAUAUUGAGUUAAAAUAAGUGUA |
| ID NO: 59) | AACUAUAAUGCCAAGUAAUUGGUUUCACUUAGGUGA (SEQ ID NO: 210) |
| 2186149 (SEQ | GUUUAAGAAAUAUGUUAGAAAUAGCAUAUUGAGUUUAAAUAAGUGUA |
| ID NO: 59) | AACUAUAAUGCCAAGUAAUUGGUUUCACUUAGGUGAAGUUAGAGCUUU |
| | UCGAAGCUCUUUU (SEQ ID NO: 211) |
| 2186167 (SEQ | GUUUAAGUACCUGGUAGGAAAUUAUCAGAUCUACUUAAACAAGGCUUU |
| ID NO: 60) | AUGCCGAAAUCAAGGACAUCGACGGAUGUCC (SEQ ID NO: 212) |
| 2186215 (SEQ | GUUUGAGAACCUUGUAGAAAUACAAGGUGAGUUCAAAUAAGGAUUUA |
| ID NO: 62) | UCCGAAAUUGCAUUUUGCCCACAUUGUGUGGAUUUAAAAAGACCGUUA |
| | GGGUCUUU (SEQ ID NO: 213) |
| 2186264 (SEQ | GUUUAAGAUCUAUGUCAGAAAUGACAUAGAGAGUUUAAAUAAAGGCU |
| ID NO: 63) | UAGCCCUAAAAAUUUACACUUAUGUGGGUGGUUGUU (SEQ ID NO: 214) |
| 2186416 (SEQ | GUUUAGCUACCAUAUGGGAAAUUAUAUGACCUGGCUAAACAAGGGUUU |
| ID NO: 65) | AUCCCGGAUUCGGCUCCUAUAAGGAGCC (SEQ ID NO: 215) |
| 2186474 (SEQ | GUUUUAGAGUUGUGUUAGAAAUAGCACAACGAGUUAAAAUAAGGUUU |
| ID NO: 66) | AACCUUAAAUGUCAAUUAAAUAUUGAUUUUCACUUA (SEQ ID NO: 216) |
| 2186573 (SEQ | GUUUAGUUACCAUAAAGGAAAUUUUAUGACCUAACUAAACAAGGGUUU |
| ID NO: 68) | AUCCCGGAUUCGGCUCUUCGGAGCC (SEQ ID NO: 217) |
| 2186752 (SEQ | AUUUAAGUACCUGUAGAGAAAUUUACAGAUCUACUUAAACAAGGCUUU |
| ID NO: 71) | AUGCCGAACUCAAGGACAUCGAAAGAUGUCC (SEQ ID NO: 218) |
| 2186752 (SEQ | AUUUUAGUACCUGAAGAGAAAUUUUCAGAUCUACUAAAACAAGGCUUU |
| ID NO: 71) | AUGCCGAACUCAAGGACAUCGAAAGAUGUCC (SEQ ID NO: 219) |
| 2186873 (SEQ | GUUUGAGAACCUUGUAGAAAACAAGUGUGAGUGCAAAUAAGGAUUUU |
| ID NO: 73) | CCGAAAUCGCCGAAAGGCCUGCAUGGUGCAGAUUAAAAGACUUGACUU |
| | UUCAAGUC (SEQ ID NO: 220) |
| 2187023 (SEQ | GUUUUAGAUUUAUGUUAGAAAUAGCAUAAAUGAGUUAAAAUAAGGUA |
| ID NO: 75) | AAACCUUAAAUGCCAAUUUAAUAUUGGUUUUGCUUA (SEQ ID NO: 221) |
| 2187023 (SEQ | GUUUAAGAUUUAUGUUAGAAAUAGCAUAAAUGAGUUUAAAUAAGGUA |
| ID NO: 75) | AAACCUUAAAUGCCAAUUUAAUAUUGGUUUUGCUUAGGCAAGUAAAGU |
| | CUUAGUUAUCUAAGGCUUUU (SEQ ID NO: 222) |
| 2187103 (SEQ | GUUUGAGAACCUUGUAGAAAUGCAAGGUGAGUUCAAAUAAGGAUUUA |
| ID NO: 76) | UCCGAAAUCGCCGUACGGCCCACAUUGUGUGGAUGAAAAGACUUGGUU |
| | UCCAGGUC (SEQ ID NO: 223) |
| 2178932 (SEQ | AUUUAAGUACCUGUAAGGAAAUUUACAGAUCUACUUAAACAAGGCUUU |
| ID NO: 6) | AUGCCGAAAUCAAGGACAUCGACGGAUGUCC (SEQ ID NO: 224) |
| 2178958 (SEQ | AUUUAAGUACCUGUAAAGAAAUUUACAGAAUCUACUUAAACAAGGCUU |
| ID NO: 7) | UAUGCCGAAAUCAAGGACAUCGACGGAUGUCC (SEQ ID NO: 225) |
| 2181613 (SEQ | GUUUUAUUACCAUUAAUGAAAUUAUAUGACCUAAUAAAACAAGGGUU |
| ID NO: 25) | UAUCCCGGAUUCGGCUCUUCGGAGCC (SEQ ID NO: 226) |
| 2186290 (SEQ | GUUUAGUUACCAUAUGAGAAAUUAUAUGACCUAACUAAACAAGGGUUU |
| ID NO: 64) | AUCCCGGAUUCGGCUCUUCGGAGCC (SEQ ID NO: 227) |
| 2182747 (SEQ | AUUUAAGUACCUAGUAGGAAAUUAUUAGAUCUACUUAAACAAGGCUUUAUGC |
| ID NO: 29) | CGAAAUCAAGGACACCGACGGGUGUCC (SEQ ID NO: 497) |
| 2184261 (SEQ | AUUUAAGUACCCGGUGAGAAAUUAUCUUCGGACCUAUUUAAACAAGGCUUUAU |
| ID NO: 46) | GCCGAAAG (SEQ ID NO: 498) |

TABLE 6 provides exemplary guide nucleic acid sequences.

TABLE 6
Exemplary Guide Nucleic Acids

| Effector | Guide Nucleic Acid- | Guide Nucleic Acid- |
|---|---|---|
| protein | DNA sequence | RNA sequence |
| 2178307 | TATTAAATACTCGTATTGCTGTTTG | UAUUAAAUACUCGUAUUGCUGUU |
| (SEQ ID NO: 1) | AGAGACTTGTAGAAATACAAGAC | UGAGAGACUUGUAGAAAUACAAG |
| | GAGTGCAAATAAGGATTTATCCGA | ACGAGUGCAAAUAAGGAUUUAUC |
| | AATCGTCGCAAGACTCTACATGGT | CGAAAUCGUCGCAAGACUCUACA |
| | GTAGAACTTAAAAGACTTGGTTTC | UGGUGUAGAACUUAAAAGACUUG |
| | AAG (SEQ ID NO: 228) | GUUUCAAG (SEQ ID NO: 327) |
| 2178521 | TATTAAATACTCGTATTGCTATTTA | UAUUAAAUACUCGUAUUGCUAUU |
| (SEQ ID NO: 2) | AGTACCTGGAGAGAAATTATCTTC | UAAGUACCUGGAGAGAAAUUAUC |
| | AGACCTACTTAAACAAGGCTTTAT | UUCAGACCUACUUAAACAAGGCU |
| | GCCGAAAG (SEQ ID NO: 229) | UUAUGCCGAAAG (SEQ ID NO: 328) |
| 2178770 | TATTAAATACTCGTATTGCTGTTTT | UAUUAAAUACUCGUAUUGCUGUU |
| (SEQ ID NO: 3) | AGAATTGTGTTAGAAATAGCACAA | UUAGAAUUGUGUUAGAAAUAGCA |
| | TGAGTTAAAATAAGGTTAAACCTT | CAAUGAGUUAAAAUAAGGUUAAA |
| | AAATGCCAGTTTTCTGGTTTCACTT | CCUUAAAUGCCAGUUUUCUGGUU |
| | AGGTG (SEQ ID NO: 230) | UCACUUAGGUG (SEQ ID NO: 329) |
| 2178770 | TATTAAATACTCGTATTGCTGTTTA | UAUUAAAUACUCGUAUUGCUGUU |
| (SEQ ID NO: 3) | AGAATTGTGTTAGAAATAGCACAA | UAAGAAUUGUGUUAGAAAUAGCA |
| | TGAGTTTAAATAAGGTTAAACCTT | CAAUGAGUUUAAAUAAGGUUAAA |
| | AAATGCCAGTTTTCTGGTTTCACTT | CCUUAAAUGCCAGUUUUCUGGUU |
| | AGGTGAATTAAGTTCTGTGTAAAC | UCACUUAGGUGAAUUAAGUUCUG |
| | AGAACTTTT (SEQ ID NO: 231) | UGUAAACAGAACUUUU (SEQ ID |
| | | NO: 330) |
| 2178869 | TATTAAATACTCGTATTGCTGTTTA | UAUUAAAUACUCGUAUUGCUGUU |
| (SEQ ID NO: 4) | AGATCTATGTCAGAAATGACATAG | UAAGAUCUAUGUCAGAAAUGACA |
| | AGAGTTTAAATAAAGGTTTAACCC | UAGAGAGUUUAAAUAAAGGUUU |
| | TAAATACTCTATTTGAGCGACTGC | AACCCUAAAUACUCUAUUUGAGC |
| | TTTGGC (SEQ ID NO: 232) | GACUGCUUUGGC (SEQ ID NO: 331) |
| 2178891 | TATTAAATACTCGTATTGCTGTTTA | UAUUAAAUACUCGUAUUGCUGUU |
| (SEQ ID NO: 5) | AGATCTATGTCAGAAATGACATAG | UAAGAUCUAUGUCAGAAAUGACA |
| | AGAGTTTAAATAAAGCTTTAAGCC | UAGAGAGUUUAAAUAAAGCUUUA |
| | TAAATGCTCACGTATGTGTTCGAA | AGCCUAAAUGCUCACGUAUGUGU |
| | CAGTTA (SEQ ID NO: 233) | UCGAACAGUUA (SEQ ID NO: 332) |
| 2178932 | TATTAAATACTCGTATTGCTATTTA | UAUUAAAUACUCGUAUUGCUAUU |
| (SEQ ID NO: 6) | AGTACCTGTAAGGAAATTTACAGA | UAAGUACCUGUAAGGAAAUUUAC |
| | TCTACTTAAACAAGGCTTTATGCC | AGAUCUACUUAAACAAGGCUUUA |
| | GAAATCAAGGACATCGACGGATG | UGCCGAAAUCAAGGACAUCGACG |
| | TCC (SEQ ID NO: 234) | GAUGUCC (SEQ ID NO: 333) |
| 2178958 | TATTAAATACTCGTATTGCTATTTA | UAUUAAAUACUCGUAUUGCUAUU |
| (SEQ ID NO: 7) | AGTACCTGTAAAGAAATTTACAGA | UAAGUACCUGUAAAGAAAUUUAC |
| | ATCTACTTAAACAAGGCTTTATGC | AGAAUCUACUUAAACAAGGCUUU |
| | CGAAATCAAGGACATCGACGGAT | AUGCCGAAAUCAAGGACAUCGAC |
| | GTCC (SEQ ID NO: 235) | GGAUGUCC (SEQ ID NO: 334) |
| 2178992 | TATTAAATACTCGTATTGCTGTTTA | UAUUAAAUACUCGUAUUGCUGUU |
| (SEQ ID NO: 8) | GTTACCATATGGGAAACTATATGA | UAGUUACCAUAUGGGAAACUAUA |
| | CCTAGCTAAACAAGGGTTTATCCC | UGACCUAGCUAAACAAGGGUUUA |
| | GGATTCGGCTCTTCGGAGCC (SEQ | UCCCGGAUUCGGCUCUUCGGAGC |
| | ID NO: 236) | C (SEQ ID NO: 335) |
| 2179018 | TATTAAATACTCGTATTGCTGTTTG | UAUUAAAUACUCGUAUUGCUGUU |
| (SEQ ID NO: 9) | AGAGTCTTGTTGAAAAACAAGAC | UGAGAGUCUUGUUGAAAAACAAG |
| | GAGTGCAAATAAGGTTTAACCGTA | ACGAGUGCAAAUAAGGUUUAACC |
| | ATCGTCGAAAGACTCTGCATGGTG | GUAAUCGUCGAAAGACUCUGCAU |
| | CAGAAAGAAAGAGGCTGTATAAA | GGUGCAGAAAGAAAGAGGCUGUA |
| | GCCT (SEQ ID NO: 237) | UAAAGCCU (SEQ ID NO: 336) |
| 2179042 | TATTAAATACTCGTATTGCTGTTTA | UAUUAAAUACUCGUAUUGCUGUU |
| (SEQ ID NO: 10) | AGATCTATGTCAGAAATGACATAG | UAAGAUCUAUGUCAGAAAUGACA |
| | AGAGTTTAAATAAAGCTTTAAGCC | UAGAGAGUUUAAAUAAAGCUUUA |
| | TAAATGCTCACATATGTGTTCAGA | AGCCUAAAUGCUCACAUAUGUGU |
| | CGGTTA (SEQ ID NO: 238) | UCAGACGGUUA (SEQ ID NO: 337) |
| 2179044 | TATTAAATACTCGTATTGCTGTTTA | UAUUAAAUACUCGUAUUGCUGUU |
| (SEQ ID NO: 11) | AGCTCTATGTTAGAAATGACATAG | UAAGCUCUAUGUUAGAAAUGACA |
| | ATAAGTTTAAATAAAAGTTTCCTA | UAGAUAAGUUUAAAUAAAAGUU |
| | TATGGATACTCTAAACACTTACTA | UCCUAUAUGGAUACUCUAAACAC |
| | TTGTAG (SEQ ID NO: 239) | UUACUAUUGUAG (SEQ ID NO: 338) |
| 2179044 | TATTAAATACTCGTATTGCTGTTTT | UAUUAAAUACUCGUAUUGCUGUU |
| (SEQ ID NO: 11) | AGCTCTATGTTAGAAATGACATAG | UUAGCUCUAUGUUAGAAAUGACA |
| | ATAAGTTAAAATAAAAGTTTCCTA | UAGAUAAGUUAAAAUAAAAGUU |
| | TATGGATACTCTAAACACTTACTA | UCCUAUAUGGAUACUCUAAACAC |
| | TTGTAGGTTGCTGCGTGTGCAGCT | UUACUAUUGUAGGUUGCUGCGUG |
| | CTTT (SEQ ID NO: 240) | UGCAGCUCUUU (SEQ ID NO: 339) |
| 2179052 | TATTAAATACTCGTATTGCTGTTAC | UAUUAAAUACUCGUAUUGCUGUU |
| (SEQ ID NO: 12) | AGTACCCTGTGAGAAATTACAGGT | ACAGUACCCUGUGAGAAAUUACA |
| | TACTGTAATAAGGCATAATGCCTT | GGUUACUGUAAUAAGGCAUAAUG |
| | AAGTGTTTGTCGACCACTACTTGT | CCUUAAGUGUUUGUCGACCACUA |
| | CGAC (SEQ ID NO: 241) | CUUGUCGAC (SEQ ID NO: 340) |
| 2179061 | TATTAAATACTCGTATTGCTGTTTA | UAUUAAAUACUCGUAUUGCUGUU |
| (SEQ ID NO: 13) | AGATCTATGTCAGAAATGACATAG | UAAGAUCUAUGUCAGAAAUGACA |
| | AGAGTTTAAATAAAGCTTTAAGCC | UAGAGAGUUUAAAUAAAGCUUUA |
| | TTAATACTCACTTAGTGAGGGACG | AGCCUUAAUACUCACUUAGUGAG |
| | GATAGC (SEQ ID NO: 242) | GGACGGAUAGC (SEQ ID NO: 341) |
| 2179107 | TATTAAATACTCGTATTGCTGTTTA | UAUUAAAUACUCGUAUUGCUGUU |
| (SEQ ID NO: 14) | AGATCTATGTCAGAAATGACATAG | UAAGAUCUAUGUCAGAAAUGACA |
| | AAAAGTTTAAATAAAGGCTTAGCC | UAGAAAAGUUUAAAUAAAGGCUU |
| | CTAAATACTCCTTTGGAGAACGAC | AGCCCUAAAUACUCCUUUGGAGA |
| | TGTGTA (SEQ ID NO: 243) | ACGACUGUGUA (SEQ ID NO: 342) |
| 2179109 | TATTAAATACTCGTATTGCTGTTTA | UAUUAAAUACUCGUAUUGCUGUU |
| (SEQ ID NO: 15) | AGATCTATGTCAGAAATGACATAG | UAAGAUCUAUGUCAGAAAUGACA |
| | AGAGTTTAAATAAAGGTTTTACCC | UAGAGAGUUUAAAUAAAGGUUU |
| | TAAATACTCTTCTGAGTTGCTGCTT | UACCCUAAAUACUCUUCUGAGUU |
| | AGGCA (SEQ ID NO: 244) | GCUGCUUAGGCA (SEQ ID NO: 343) |
| 2180100 | TATTAAATACTCGTATTGCTGTTTA | UAUUAAAUACUCGUAUUGCUGUU |
| (SEQ ID NO: 16) | GTTACCATATGGGAAACTATATGA | UAGUUACCAUAUGGGAAACUAUA |
| | CCTAACTAAACAAGGGTTTATCCC | UGACCUAACUAAACAAGGGUUUA |
| | GGATTCGGCTCTTCGGAGCC (SEQ | UCCCGGAUUCGGCUCUUCGGAGC |
| | ID NO: 245) | C (SEQ ID NO: 344) |
| 2180100 | TATTAAATACTCGTATTGCTGTTTT | UAUUAAAUACUCGUAUUGCUGUU |
| (SEQ ID NO: 16) | GTTACCATATGGGAAACTATATGA | UUGUUACCAUAUGGGAAACUAUA |
| | CCTAACAAAACAAGGGTTTATCCC | UGACCUAACAAAACAAGGGUUUA |
| | GGATTCGGCTCTTCGGAGCC (SEQ | UCCCGGAUUCGGCUCUUCGGAGC |
| | ID NO: 246) | C (SEQ ID NO: 345) |
| 2180475 | TATTAAATACTCGTATTGCTGTTTT | UAUUAAAUACUCGUAUUGCUGUU |
| (SEQ ID NO: 17) | AGAAGTATGTTAGAAATAACATAC | UUAGAAGUAUGUUAGAAAUAACA |
| | CGAGTTAAAATAAGGTTTAACCTT | UACCGAGUUAAAAUAAGGUUUAA |
| | AAATACTGGCTATGCCAGATTCAT | CCUUAAAUACUGGCUAUGCCAGA |
| | TTAGAT (SEQ ID NO: 247) | UUCAUUUAGAU (SEQ ID NO: 346) |
| 2180475 | TATTAAATACTCGTATTGCTGTTTA | UAUUAAAUACUCGUAUUGCUGUU |
| (SEQ ID NO: 17) | AGAAGTATGTTAGAAATAACATAC | UAAGAAGUAUGUUAGAAAUAACA |
| | CGAGTTTAAATAAGGTTTAACCTT | UACCGAGUUUAAAUAAGGUUUAA |
| | AAATACTGGCTATGCCAGATTCAT | CCUUAAAUACUGGCUAUGCCAGA |
| | TTAGATGAAGTAGCGCCTCTTTGG | UUCAUUUAGAUGAAGUAGCGCCU |
| | AGGTGCTTTA (SEQ ID NO: 248) | CUUUGGAGGUGCUUUA (SEQ ID |
| | | NO: 347) |
| 2180553 | TATTAAATACTCGTATTGCTGTTAC | UAUUAAAUACUCGUAUUGCUGUU |
| (SEQ ID NO: 18) | AGGACCCTGTATGAAAATACAGGT | ACAGGACCCUGUAUGAAAAUACA |
| | TTCTGTAATAAGGCATAATGCCTT | GGUUUCUGUAAUAAGGCAUAAUG |
| | AGGCTTAATCCCCCTTAAGGATTT | CCUUAGGCUUAAUCCCCCUUAAG |
| | (SEQ ID NO: 249) | GAUUU (SEQ ID NO: 348) |
| 2180948 | TATTAAATACTCGTATTGCTGTTTA | UAUUAAAUACUCGUAUUGCUGUU |
| (SEQ ID NO: 19) | AGTTCTATGTCAGAAATGACATAG | UAAGUUCUAUGUCAGAAAUGACA |
| | AGAGTTTAAATAAAGGATTACCTA | UAGAGAGUUUAAAUAAAGGAUU |
| | ATTACTCATTATGTGATGAGAGGC | ACCUAAUUACUCAUUAUGUGAUG |
| | TGCTTA (SEQ ID NO: 250) | AGAGGCUGCUUA (SEQ ID NO: 349) |
| 2180948 | TATTAAATACTCGTATTGCTGTTTT | UAUUAAAUACUCGUAUUGCUGUU |
| (SEQ ID NO: 19) | AGTTCTATGTCAGAAATGACATAG | UUAGUUCUAUGUCAGAAAUGACA |
| | AGAGTTAAAATAAAGGATTACCTA | UAGAGAGUUAAAAUAAAGGAUU |
| | ATTACTCATTATGTGATGAGAGGC | ACCUAAUUACUCAUUAUGUGAUG |
| | TGCTTAGGCTGGCCTTTT (SEQ ID | AGAGGCUGCUUAGGCUGGCCUUU |
| | NO: 251) | U (SEQ ID NO: 350) |
| 2181249 | TATTAAATACTCGTATTGCTGTTTT | UAUUAAAUACUCGUAUUGCUGUU |
| (SEQ ID NO: 20) | AGAATTATGTTAGAAATAGCATAA | UUAGAAUUAUGUUAGAAAUAGCA |
| | TGAGTTAAAATAAGGTTAAACCTT | UAAUGAGUUAAAAUAAGGUUAA |
| | TAATGCTGATTTATATCAGTTTCGC | ACCUUUAAUGCUGAUUUAUAUCA |
| | TTAGG (SEQ ID NO: 252) | GUUUCGCUUAGG (SEQ ID NO: 351) |
| 2181249 | TATTAAATACTCGTATTGCTGTTTA | UAUUAAAUACUCGUAUUGCUGUU |
| (SEQ ID NO: 20) | AGAATTATGTTAGAAATAGCATAA | UAAGAAUUAUGUUAGAAAUAGCA |
| | TGAGTTTAAATAAGGTTAAACCTT | UAAUGAGUUUAAAUAAGGUUAA |
| | TAATGCTGATTTATATCAGTTTCGC | ACCUUUAAUGCUGAUUUAUAUCA |
| | TTAGGTGAAATGAGCTCTTTGAGC | GUUUCGCUUAGGUGAAAUGAGCU |
| | TCTTTTT (SEQ ID NO: 253) | CUUUGAGCUCUUUUU (SEQ ID NO: |
| | | 352) |
| 2181365 | TATTAAATACTCGTATTGCTGTTTA | UAUUAAAUACUCGUAUUGCUGUU |
| (SEQ ID NO: 21) | AGATCTATGTCAGAAATGACATAG | UAAGAUCUAUGUCAGAAAUGACA |
| | AGAGTTTAAATAAAGCTTTAAGCC | UAGAGAGUUUAAAUAAAGCUUUA |
| | TAAATGCTCACTTGTGTTCGGACG | AGCCUAAAUGCUCACUUGUGUUC |
| | GTTAGC (SEQ ID NO: 254) | GGACGGUUAGC (SEQ ID NO: 353) |
| 2181445 | TATTAAATACTCGTATTGCTGTTTG | UAUUAAAUACUCGUAUUGCUGUU |
| (SEQ ID NO: 22) | AGAGTATTGTAGAAATACAAGAC | UGAGAGUAUUGUAGAAAUACAAG |
| | GAGTGCAAATAAGGTTTATCCGAA | ACGAGUGCAAAUAAGGUUUAUCC |
| | ATCGTCGTATGACTCTACATGGTG | GAAAUCGUCGUAUGACUCUACAU |
| | TAGATAATAAAAAGGACTTGCGTG | GGUGUAGAUAAUAAAAAGGACUU |
| | CAA (SEQ ID NO: 255) | GCGUGCAA (SEQ ID NO: 354) |
| 2181509 | TATTAAATACTCGTATTGCTGTTTG | UAUUAAAUACUCGUAUUGCUGUU |
| (SEQ ID NO: 23) | AGAGACTTGTAGAAATACAAGAC | UGAGAGACUUGUAGAAAUACAAG |
| | GAGTGCAAATAAGGATTTATCCGA | ACGAGUGCAAAUAAGGAUUUAUC |
| | AATCGTCGCAAGACTCTACATGGT | CGAAAUCGUCGCAAGACUCUACA |
| | GTAGAACTTAAAAGGCTTGGTTTC | UGGUGUAGAACUUAAAAGGCUUG |
| | AAG (SEQ ID NO: 256) | GUUUCAAG (SEQ ID NO: 355) |
| 2181587 | TATTAAATACTCGTATTGCTGTTTG | UAUUAAAUACUCGUAUUGCUGUU |
| (SEQ ID NO: 24) | AGAACCTTGTAGAAATACAAGGT | UGAGAACCUUGUAGAAAUACAAG |
| | GAGTTCAAATAAGGATTTTATCCG | GUGAGUUCAAAUAAGGAUUUUAU |
| | ACATCGCCGTAAGGTTAGCATTGT | CCGACAUCGCCGUAAGGUUAGCA |
| | GCGACAGAAAAGACTTCTTCGGA | UUGUGCGACAGAAAAGACUUCUU |
| | AGTC (SEQ ID NO: 257) | CGGAAGUC (SEQ ID NO: 356) |
| 2181613 | TATTAAATACTCGTATTGCTGTTTA | UAUUAAAUACUCGUAUUGCUGUU |
| (SEQ ID NO: 25) | ATTACCATTAATGAAATTATATGA | UAAUUACCAUUAAUGAAAUUAUA |
| | CCTAATTAAACAAGGGTTTATCCC | UGACCUAAUUAAACAAGGGUUUA |
| | GGATTCGGCTCTTCGGAGCC (SEQ | UCCCGGAUUCGGCUCUUCGGAGC |
| | ID NO: 258) | C (SEQ ID NO: 357) |
| 2181613 | TATTAAATACTCGTATTGCTGTTTT | UAUUAAAUACUCGUAUUGCUGUU |
| (SEQ ID NO: 25) | ATTACCATTAATGAAATTATATGA | UUAUUACCAUUAAUGAAAUUAUA |
| | CCTAATAAAACAAGGGTTTATCCC | UGACCUAAUAAAACAAGGGUUUA |
| | GGATTCGGCTCTTCGGAGCC (SEQ | UCCCGGAUUCGGCUCUUCGGAGC |
| | ID NO: 259) | C (SEQ ID NO: 358) |
| 2181731 | TATTAAATACTCGTATTGCTGTTTT | UAUUAAAUACUCGUAUUGCUGUU |
| (SEQ ID NO: 26) | AGAAGTATGTTAGAAATAACATAC | UUAGAAGUAUGUUAGAAAUAACA |
| | TGAGTTAAAATAAGTGTAAACTTA | UACUGAGUUAAAAUAAGUGUAAA |
| | ACCACTGATTTATCAGTTCTACTT | CUUAACCACUGAUUUAUCAGUUC |
| | AGGTAG (SEQ ID NO: 260) | UACUUAGGUAG (SEQ ID NO: 359) |
| 2181731 | TATTAAATACTCGTATTGCTGTTTA | UAUUAAAUACUCGUAUUGCUGUU |
| (SEQ ID NO: 26) | AGAAGTATGTTAGAAATAACATAC | UAAGAAGUAUGUUAGAAAUAACA |
| | TGAGTTTAAATAAGTGTAAACTTA | UACUGAGUUUAAAUAAGUGUAAA |
| | ACCACTGATTTATCAGTTCTACTT | CUUAACCACUGAUUUAUCAGUUC |
| | AGGTAGAATAAAGTCCTATTTATA | UACUUAGGUAGAAUAAAGUCCUA |
| | GGGCTTTT (SEQ ID NO: 261) | UUUAUAGGGCUUUU (SEQ ID NO: |
| | | 360) |
| 2182400 | TATTAAATACTCGTATTGCTGTTTG | UAUUAAAUACUCGUAUUGCUGUU |
| (SEQ ID NO: 27) | AGAGCCTTGTTGAAAAACAAGGC | UGAGAGCCUUGUUGAAAAACAAG |
| | GAGTTCAAATAAGGATTATTCCGA | GCGAGUUCAAAUAAGGAUUAUUC |
| | AACCGCTTTATAGTCTGCATTGTG | CGAAACCGCUUUAUAGUCUGCAU |
| | CAGGTTCTCATTGATCCGGCGAAG | UGUGCAGGUUCUCAUUGAUCCGG |
| | GCC (SEQ ID NO: 262) | CGAAGGCC (SEQ ID NO: 361) |
| 2182512 | TATTAAATACTCGTATTGCTATTTA | UAUUAAAUACUCGUAUUGCUAUU |
| (SEQ ID NO: 28) | AGTACCTGTAAGGAAATTTACAGA | UAAGUACCUGUAAGGAAAUUUAC |
| | TCTACTTAAACAAGGCTTTATGCC | AGAUCUACUUAAACAAGGCUUUA |
| | GAAATCAAGGACACGCTTGCGTGT | UGCCGAAAUCAAGGACACGCUUG |
| | CC (SEQ ID NO: 263) | CGUGUCC (SEQ ID NO: 362) |
| 2182512 | TATTAAATACTCGTATTGCTATTTT | UAUUAAAUACUCGUAUUGCUAUU |
| (SEQ ID NO: 28) | AGTACCTGAAAGGAAATTTTCAGA | UUAGUACCUGAAAGGAAAUUUUC |
| | TCTACTAAAACAAGGCTTTATGCC | AGAUCUACUAAAACAAGGCUUUA |
| | GAAATCAAGGACACGCTTGCGTGT | UGCCGAAAUCAAGGACACGCUUG |
| | CC (SEQ ID NO: 264) | CGUGUCC (SEQ ID NO: 363) |
| 2182747 | TATTAAATACTCGTATTGCTATTTA | UAUUAAAUACUCGUAUUGCUAUU |
| (SEQ ID NO: 29) | AGTACCTAGTAGGAAATATTAGAT | UAAGUACCUAGUAGGAAAUAUUA |
| | CTACTTAAACAAGGCTTTATGCCG | GAUCUACUUAAACAAGGCUUUAU |
| | AAATCAAGGACACCGACGGGTGT | GCCGAAAUCAAGGACACCGACGG |
| | CC (SEQ ID NO: 265) | GUGUCC (SEQ ID NO: 364) |
| 2182914 | TATTAAATACTCGTATTGCTGTTTG | UAUUAAAUACUCGUAUUGCUGUU |
| (SEQ ID NO: 30) | AGAACCTTGTAGAAAACAAGGTG | UGAGAACCUUGUAGAAAACAAGG |
| | AGTGCAAATAAGGATTTTCCGAAA | UGAGUGCAAAUAAGGAUUUUCCG |
| | TCGCCGTAAGGCTCTGCATGGTGC | AAAUCGCCGUAAGGCUCUGCAUG |
| | AGATTCAAAAGGCCTGTTTACAGG | GUGCAGAUUCAAAAGGCCUGUUU |
| | TCT (SEQ ID NO: 266) | ACAGGUCU (SEQ ID NO: 365) |
| 2183116 | TATTAAATACTCGTATTGCTGTTTG | UAUUAAAUACUCGUAUUGCUGUU |
| (SEQ ID NO: 31) | AGAGCCCTGTAGAAATACAGGGT | UGAGAGCCCUGUAGAAAUACAGG |
| | GAGTTCAAATAAGGCTTTAGCCGA | GUGAGUUCAAAUAAGGCUUUAGC |
| | AATCGCGCCTCGTGTCCGCATTGT | CGAAAUCGCGCCUCGUGUCCGCA |
| | GCGGGTTTAAAAGAGAATCGTAA | UUGUGCGGGUUUAAAAGAGAAUC |
| | GATT (SEQ ID NO: 267) | GUAAGAUU (SEQ ID NO: 366) |
| 2183137 | TATTAAATACTCGTATTGCTGTTTG | UAUUAAAUACUCGUAUUGCUGUU |
| (SEQ ID NO: 32) | AGAACCTTGTAGAAATACAAGGT | UGAGAACCUUGUAGAAAUACAAG |
| | GAGTTCAAATAAGGAGCAATCCG | GUGAGUUCAAAUAAGGAGCAAUC |
| | AAATTACATTGACCGCATTGTGCG | CGAAAUUACAUUGACCGCAUUGU |
| | GATATGAAAGACGTAGCCTATGGC | GCGGAUAUGAAAGACGUAGCCUA |
| | TAAG (SEQ ID NO: 268) | UGGCUAAG (SEQ ID NO: 367) |
| 2183156 | TATTAAATACTCGTATTGCTGTTTG | UAUUAAAUACUCGUAUUGCUGUU |
| (SEQ ID NO: 33) | AGAACCTTGTAGAAAACAAGGTG | UGAGAACCUUGUAGAAAACAAGG |
| | AGTTCAAATAAGGATTTATCCGAA | UGAGUUCAAAUAAGGAUUUAUCC |
| | ATTACATTTGCCCGCATTGTGCGG | GAAAUUACAUUUGCCCGCAUUGU |
| | ATTGAAAAGGTTCGACTTATGTCG | GCGGAUUGAAAAGGUUCGACUUA |
| | AAT (SEQ ID NO: 269) | UGUCGAAU (SEQ ID NO: 368) |
| 2183168 | TATTAAATACTCGTATTGCTGTTTA | UAUUAAAUACUCGUAUUGCUGUU |
| (SEQ ID NO: 34) | AGTACCTAGTAAGAAATTATTAGA | UAAGUACCUAGUAAGAAAUUAUU |
| | CCTACTTAAATAAGGCTTTATGCC | AGACCUACUUAAAUAAGGCUUUA |
| | GAAATCAAGAACACCCGACGGGG | UGCCGAAAUCAAGAACACCCGAC |
| | TGTTC (SEQ ID NO: 270) | GGGGUGUUC (SEQ ID NO: 369) |
| 2183254 | TATTAAATACTCGTATTGCTGTTTA | UAUUAAAUACUCGUAUUGCUGUU |
| (SEQ ID NO: 35) | AGTACCTGGTAGGAAATTATCAGA | UAAGUACCUGGUAGGAAAUUAUC |
| | TCTACTTAAACAAGGCTTTATGCC | AGAUCUACUUAAACAAGGCUUUA |
| | GAAATCAAGGACATCGACGGATG | UGCCGAAAUCAAGGACAUCGACG |
| | TCC (SEQ ID NO: 271) | GAUGUCC (SEQ ID NO: 370) |
| 2183262 | TATTAAATACTCGTATTGCTGTTTG | UAUUAAAUACUCGUAUUGCUGUU |
| (SEQ ID NO: 36) | AGAACCCTGTAGAAATACAGGGT | UGAGAACCCUGUAGAAAUACAGG |
| | GAGTTCAAATAAGGCATTAGCCGA | GUGAGUUCAAAUAAGGCAUUAGC |
| | AATTGCACCTGATGTCCGCATTGT | CGAAAUUGCACCUGAUGUCCGCA |
| | GCGGGATTAAAGAGGATCGAAGG | UUGUGCGGGAUUAAAGAGGAUCG |
| | ATTC (SEQ ID NO: 272) | AAGGAUUC (SEQ ID NO: 371) |
| 2183288 | TATTAAATACTCGTATTGCTGTTTG | UAUUAAAUACUCGUAUUGCUGUU |
| (SEQ ID NO: 37) | AGAGCCCTGTAGAAATACAGGGT | UGAGAGCCCUGUAGAAAUACAGG |
| | GAGTTCAAATAAGGCTTTAGCCGA | GUGAGUUCAAAUAAGGCUUUAGC |
| | TATCGCACCTCGTGTCCGCATTGT | CGAUAUCGCACCUCGUGUCCGCA |
| | GCGGGATTTTAAGAGAATCGTTTC | UUGUGCGGGAUUUUAAGAGAAUC |
| | GAT (SEQ ID NO: 273) | GUUUCGAU (SEQ ID NO: 372) |
| 2183304 | TATTAAATACTCGTATTGCTGTTTG | UAUUAAAUACUCGUAUUGCUGUU |
| (SEQ ID NO: 38) | AGAGACTTGTTGAAAAACAGGTC | UGAGAGACUUGUUGAAAAACAGG |
| | GAGTGCAAATAAGGTTTATCCGAA | UCGAGUGCAAAUAAGGUUUAUCC |
| | CTCGTCAATTTGATCCGCATTGTG | GAACUCGUCAAUUUGAUCCGCAU |
| | CGGTTTTTAAAATCCGATGTTTCAT | UGUGCGGUUUUUAAAAUCCGAUG |
| | CG (SEQ ID NO: 274) | UUUCAUCG (SEQ ID NO: 373) |
| 2183304 | TATTAAATACTCGTATTGCTGTTTG | UAUUAAAUACUCGUAUUGCUGUU |
| (SEQ ID NO: 38) | AGAGACTTGTTGAAAAACAGGTC | UGAGAGACUUGUUGAAAAACAGG |
| | GAGTGCAAATAAGGTTTATCCGAA | UCGAGUGCAAAUAAGGUUUAUCC |
| | CTCGTCAATTTGATCCGCATTGTG | GAACUCGUCAAUUUGAUCCGCAU |
| | CGGTTATTAAAATCCGATGTTTCA | UGUGCGGUUAUUAAAAUCCGAUG |
| | TCG (SEQ ID NO: 275) | UUUCAUCG (SEQ ID NO: 374) |
| 2183312 | TATTAAATACTCGTATTGCTGTTTA | UAUUAAAUACUCGUAUUGCUGUU |
| (SEQ ID NO: 39) | AGTACCTGGAGAGAAATTTACTTC | UAAGUACCUGGAGAGAAAUUUAC |
| | AGACCTACTTAAATAAGGATTTAT | UUCAGACCUACUUAAAUAAGGAU |
| | TCCGAAAGAA (SEQ ID NO: 276) | UUAUUCCGAAAGAA (SEQ ID NO: |
| | | 375) |
| 2183526 | TATTAAATACTCGTATTGCTGTTTG | UAUUAAAUACUCGUAUUGCUGUU |
| (SEQ ID NO: 40) | AGAGCCTTGTTGAAAAACAAGGC | UGAGAGCCUUGUUGAAAAACAAG |
| | GAGTTCAAATAAGGATTTTTCCGA | GCGAGUUCAAAUAAGGAUUUUUC |
| | AATCGCTATTTAGTCTGCAGTGTG | CGAAAUCGCUAUUUAGUCUGCAG |
| | CAGTTTCGTGTCCGGCTTTGCCGG | UGUGCAGUUUCGUGUCCGGCUUU |
| | GCA (SEQ ID NO: 277) | GCCGGGCA (SEQ ID NO: 376) |
| 2183526 | TATTAAATACTCGTATTGCTGTTTG | UAUUAAAUACUCGUAUUGCUGUU |
| (SEQ ID NO: 40) | AGAGCCTTGTTGAAAAACAAGGC | UGAGAGCCUUGUUGAAAAACAAG |
| | GAGTTCAAATAAGGATTATTCCGA | GCGAGUUCAAAUAAGGAUUAUUC |
| | AATCGCTATTTAGTCTGCAGTGTG | CGAAAUCGCUAUUUAGUCUGCAG |
| | CAGTTTCGTGTCCGGCTTTGCCGG | UGUGCAGUUUCGUGUCCGGCUUU |
| | GCA (SEQ ID NO: 278) | GCCGGGCA (SEQ ID NO: 377) |
| 2183548 | TATTAAATACTCGTATTGCTGTTTG | UAUUAAAUACUCGUAUUGCUGUU |
| (SEQ ID NO: 41) | AGAGCTTTGTTGAAAAACAAAGC | UGAGAGCUUUGUUGAAAAACAAA |
| | GAGTGCAAATAAGGTTTATCCGAA | GCGAGUGCAAAUAAGGUUUAUCC |
| | ATCGCTTTTATAGCAAGCATTGTG | GAAAUCGCUUUUAUAGCAAGCAU |
| | CTTATTAAATCTGGCTTTCCAGCC | UGUGCUUAUUAAAUCUGGCUUUC |
| | AGA (SEQ ID NO: 279) | CAGCCAGA (SEQ ID NO: 378) |
| 2183594 | TATTAAATACTCGTATTGCTGTTTG | UAUUAAAUACUCGUAUUGCUGUU |
| (SEQ ID NO: 42) | AGAGCTTTGTTGAAAAACAAAGC | UGAGAGCUUUGUUGAAAAACAAA |
| | GAGTGCAAATAAGGTTTATCCGAA | GCGAGUGCAAAUAAGGUUUAUCC |
| | ATCGCTTTTATAGCAAGCATCGTG | GAAAUCGCUUUUAUAGCAAGCAU |
| | CTTATTAAATCTGGCTTTTTAGCCA | CGUGCUUAUUAAAUCUGGCUUUU |
| | GA (SEQ ID NO: 280) | UAGCCAGA (SEQ ID NO: 379) |
| 2183594 | TATTAAATACTCGTATTGCTGTTTG | UAUUAAAUACUCGUAUUGCUGUU |
| (SEQ ID NO: 42) | AGAGCTTTGTTGAAAAACAAAGC | UGAGAGCUUUGUUGAAAAACAAA |
| | GAGTGCAAATAAGGTTTATCCGAA | GCGAGUGCAAAUAAGGUUUAUCC |
| | ATCGCTTTTATAGCAAGCATCGTG | GAAAUCGCUUUUAUAGCAAGCAU |
| | CTTATTAAATCTGGCTTATTAGCC | CGUGCUUAUUAAAUCUGGCUUAU |
| | AGA (SEQ ID NO: 281) | UAGCCAGA (SEQ ID NO: 380) |
| 2183638 | TATTAAATACTCGTATTGCTGTTTG | UAUUAAAUACUCGUAUUGCUGUU |
| (SEQ ID NO: 43) | AGAGCCCTGTAGAAATACAGGGT | UGAGAGCCCUGUAGAAAUACAGG |
| | GAGTTCAAATAAGGCTTTAGCCGA | GUGAGUUCAAAUAAGGCUUUAGC |
| | AATCGCACCTCGTGTCCGCATTGT | CGAAAUCGCACCUCGUGUCCGCA |
| | GCGGGTTTAAAGAGAATCGTTTGA | UUGUGCGGGUUUAAAGAGAAUCG |
| | TTC (SEQ ID NO: 282) | UUUGAUUC (SEQ ID NO: 381) |
| 2183868 | TATTAAATACTCGTATTGCTGTTTG | UAUUAAAUACUCGUAUUGCUGUU |
| (SEQ ID NO: 44) | AGAGCCCTGTAGAAATACAGGGT | UGAGAGCCCUGUAGAAAUACAGG |
| | GAGTTCAAATAAGGCTTTAGCCGA | GUGAGUUCAAAUAAGGCUUUAGC |
| | AATCGCACCTGAGTGTCCGCATTG | CGAAAUCGCACCUGAGUGUCCGC |
| | TGCGGGATTAAGAGAATCGAAAG | AUUGUGCGGGAUUAAGAGAAUCG |
| | ATTC (SEQ ID NO: 283) | AAAGAUUC (SEQ ID NO: 382) |
| 2184029 | TATTAAATACTCGTATTGCTGTTTG | UAUUAAAUACUCGUAUUGCUGUU |
| (SEQ ID NO: 45) | AGAACCTTGTAGAAATACAAGGT | UGAGAACCUUGUAGAAAUACAAG |
| | GAGTTCAAATAAGGATTTATCCGA | GUGAGUUCAAAUAAGGAUUUAUC |
| | CATCGCCGTAAGGCCCACATTGTG | CGACAUCGCCGUAAGGCCCACAU |
| | TGGTTTAAAAGACTTGAGAAAGTC | UGUGUGGUUUAAAAGACUUGAGA |
| | TTT (SEQ ID NO: 284) | AAGUCUUU (SEQ ID NO: 383) |
| 2184261 | TATTAAATACTCGTATTGCTATTTA | UAUUAAAUACUCGUAUUGCUAUU |
| (SEQ ID NO: 46) | AGTACCCGGTGAGAAAATTTCTTC | UAAGUACCCGGUGAGAAAAUUUC |
| | GGACCTATTTAAACAAGGCTTTAT | UUCGGACCUAUUUAAACAAGGCU |
| | GCCGAAAG (SEQ ID NO: 285) | UUAUGCCGAAAG (SEQ ID NO: 384) |
| 2184558 | TATTAAATACTCGTATTGCTGTTTA | UAUUAAAUACUCGUAUUGCUGUU |
| (SEQ ID NO: 47) | GTTACCATAAGGGAAATTTTATGA | UAGUUACCAUAAGGGAAAUUUUA |
| | CCTAACTAAACAAGGGTTTATCCC | UGACCUAACUAAACAAGGGUUUA |
| | GGATTCGGCTCTTCGGAGCC (SEQ | UCCCGGAUUCGGCUCUUCGGAGC |
| | ID NO: 286) | C (SEQ ID NO: 385) |
| 2184558 | TATTAAATACTCGTATTGCTGTTTT | UAUUAAAUACUCGUAUUGCUGUU |
| (SEQ ID NO: 47) | GTTACCATAAGGGAAATTTTATGA | UUGUUACCAUAAGGGAAAUUUUA |
| | CCTAACAAAACAAGGGTTTATCCC | UGACCUAACAAAACAAGGGUUUA |
| | GGATTCGGCTCTTCGGAGCC (SEQ | UCCCGGAUUCGGCUCUUCGGAGC |
| | ID NO: 287) | C (SEQ ID NO: 386) |
| 2184818 | TATTAAATACTCGTATTGCTGTTTA | UAUUAAAUACUCGUAUUGCUGUU |
| (SEQ ID NO: 48) | GTTACCATAAAGGAAATTTTATGA | UAGUUACCAUAAAGGAAAUUUUA |
| | CCTAACTAAACAAGGGTTTATCCC | UGACCUAACUAAACAAGGGUUUA |
| | GGATTCGGCTCTTCGGAGCC (SEQ | UCCCGGAUUCGGCUCUUCGGAGC |
| | ID NO: 288) | C (SEQ ID NO: 387) |
| 2185070 | TATTAAATACTCGTATTGCTGTTTT | UAUUAAAUACUCGUAUUGCUGUU |
| (SEQ ID NO: 49) | AGTATTGTGTTAGAAATAGCACAA | UUAGUAUUGUGUUAGAAAUAGCA |
| | TGAGTTAAAATAAGGTTTAAACCT | CAAUGAGUUAAAAUAAGGUUUAA |
| | TAAATGCCAACTTGATGTTGGTTT | ACCUUAAAUGCCAACUUGAUGUU |
| | CACTTA (SEQ ID NO: 289) | GGUUUCACUUA (SEQ ID NO: 388) |
| 2185070 | TATTAAATACTCGTATTGCTGTTTA | UAUUAAAUACUCGUAUUGCUGUU |
| (SEQ ID NO: 49) | AGTATTGTGTTAGAAATAGCACAA | UAAGUAUUGUGUUAGAAAUAGCA |
| | TGAGTTTAAATAAGGTTTAAACCT | CAAUGAGUUUAAAUAAGGUUUAA |
| | TAAATGCCAACTTGATGTTGGTTT | ACCUUAAAUGCCAACUUGAUGUU |
| | CACTTAGGTGAATTTAAGTTCTAG | GGUUUCACUUAGGUGAAUUUAAG |
| | TTATCTAGAGCTTTT (SEQ ID NO: | UUCUAGUUAUCUAGAGCUUUU |
| | 290) | (SEQ ID NO: 389) |
| 2185320 | TATTAAATACTCGTATTGCTATTTA | UAUUAAAUACUCGUAUUGCUAUU |
| (SEQ ID NO: 50) | AGTACCTGGTAAGAAATTATCAGA | UAAGUACCUGGUAAGAAAUUAUC |
| | TCTACTTAAACAAGGCTTTATGCC | AGAUCUACUUAAACAAGGCUUUA |
| | GAAATCAAGGACATCGATGGATG | UGCCGAAAUCAAGGACAUCGAUG |
| | TCC (SEQ ID NO: 291) | GAUGUCC (SEQ ID NO: 390) |
| 2185320 | TATTAAATACTCGTATTGCTATTTT | UAUUAAAUACUCGUAUUGCUAUU |
| (SEQ ID NO: 50) | AGTACCTGGAAAGAAATTTTCAGA | UUAGUACCUGGAAAGAAAUUUUC |
| | TCTACTAAAACAAGGCTTTATGCC | AGAUCUACUAAAACAAGGCUUUA |
| | GAAATCAAGGACATCGATGGATG | UGCCGAAAUCAAGGACAUCGAUG |
| | TCC (SEQ ID NO: 292) | GAUGUCC (SEQ ID NO: 391) |
| 2185440 | TATTAAATACTCGTATTGCTATTTA | UAUUAAAUACUCGUAUUGCUAUU |
| (SEQ ID NO: 51) | AGTACCTGTAAGGAAATTTACAGA | UAAGUACCUGUAAGGAAAUUUAC |
| | TCTACTTAAATAAGGCTTTATGCC | AGAUCUACUUAAAUAAGGCUUUA |
| | GAAATCAAGGAGTGCCGACGGGC | UGCCGAAAUCAAGGAGUGCCGAC |
| | ACTCC (SEQ ID NO: 293) | GGGCACUCC (SEQ ID NO: 392) |
| 2185446 | TATTAAATACTCGTATTGCTATTTA | UAUUAAAUACUCGUAUUGCUAUU |
| (SEQ ID NO: 52) | AGTACCTGGTAGGAAATTATCAGA | UAAGUACCUGGUAGGAAAUUAUC |
| | TCTACTTAAACAAGGCTTTATGCC | AGAUCUACUUAAACAAGGCUUUA |
| | GAAATGAAAAGGACATCCTCGGA | UGCCGAAAUGAAAAGGACAUCCU |
| | TGTCC (SEQ ID NO: 294) | CGGAUGUCC (SEQ ID NO: 393) |
| 2185499 | TATTAAATACTCGTATTGCTGTTTT | UAUUAAAUACUCGUAUUGCUGUU |
| (SEQ ID NO: 53) | AGAATTGTGTTAGAAATAGCACAA | UUAGAAUUGUGUUAGAAAUAGCA |
| | TGAGTTAAAATAAGGGTAACCTTA | CAAUGAGUUAAAAUAAGGGUAAC |
| | AATGCCAATTCATATTGGTTTCAC | CUUAAAUGCCAAUUCAUAUUGGU |
| | TTAGGT (SEQ ID NO: 295) | UUCACUUAGGU (SEQ ID NO: 394) |
| 2185499 | TATTAAATACTCGTATTGCTGTTTA | UAUUAAAUACUCGUAUUGCUGUU |
| (SEQ ID NO: 53) | AGAATTGTGTTAGAAATAGCACAA | UAAGAAUUGUGUUAGAAAUAGCA |
| | TGAGTTTAAATAAGGGTAACCTTA | CAAUGAGUUUAAAUAAGGGUAAC |
| | AATGCCAATTCATATTGGTTTCAC | CUUAAAUGCCAAUUCAUAUUGGU |
| | TTAGGTGAATAAAGAGTTCTAGCT | UUCACUUAGGUGAAUAAAGAGUU |
| | TGCTAGGGCTTTT (SEQ ID NO: 296) | CUAGCUUGCUAGGGCUUUU (SEQ |
| | | ID NO: 395) |
| 2185882 | TATTAAATACTCGTATTGCTGTTTG | UAUUAAAUACUCGUAUUGCUGUU |
| (SEQ ID NO: 54) | AGAACCTTGTAGAAATACAAGGT | UGAGAACCUUGUAGAAAUACAAG |
| | GAGTGCAAATAAGGATTTATCCGA | GUGAGUGCAAAUAAGGAUUUAUC |
| | CATCGCCGTTTGGTCTGCATGGTG | CGACAUCGCCGUUUGGUCUGCAU |
| | CAGGTAAAAGACTTGGTTTCAAGT | GGUGCAGGUAAAAGACUUGGUUU |
| | CTT (SEQ ID NO: 297) | CAAGUCUU (SEQ ID NO: 396) |
| 2185938 | TATTAAATACTCGTATTGCTGTTTA | UAUUAAAUACUCGUAUUGCUGUU |
| (SEQ ID NO: 55) | AGTTCTATGTCAGAAATGACATAG | UAAGUUCUAUGUCAGAAAUGACA |
| | AGAGTTTAAATAAAGGTTTTACCC | UAGAGAGUUUAAAUAAAGGUUU |
| | TAAATGCTCACGTATGTGTTCGGC | UACCCUAAAUGCUCACGUAUGUG |
| | TGCTTA (SEQ ID NO: 298) | UUCGGCUGCUUA (SEQ ID NO: 397) |
| 2185986 | TATTAAATACTCGTATTGCTATTTA | UAUUAAAUACUCGUAUUGCUAUU |
| (SEQ ID NO: 56) | AGTACCTGTAAGGAAATTTACAGA | UAAGUACCUGUAAGGAAAUUUAC |
| | TCTACTTAAATAAGGCTTTATGCC | AGAUCUACUUAAAUAAGGCUUUA |
| | GAGATTAAGGAGTGCCGACGGGT | UGCCGAGAUUAAGGAGUGCCGAC |
| | ACTCC (SEQ ID NO: 299) | GGGUACUCC (SEQ ID NO: 398) |
| 2186029 | TATTAAATACTCGTATTGCTGTTTA | UAUUAAAUACUCGUAUUGCUGUU |
| (SEQ ID NO: 57) | GCTACCATCTATGAAAGATATATG | UAGCUACCAUCUAUGAAAGAUAU |
| | ATCTAGCTAAACAAGGGTTTATCC | AUGAUCUAGCUAAACAAGGGUUU |
| | CGGATTCGGCTCCTATAAGGAGCC | AUCCCGGAUUCGGCUCCUAUAAG |
| | (SEQ ID NO: 300) | GAGCC (SEQ ID NO: 399) |
| 2186035 | TATTAAATACTCGTATTGCTGTTTT | UAUUAAAUACUCGUAUUGCUGUU |
| (SEQ ID NO: 58) | AGTATTGTGTTAGAAATAGCACAA | UUAGUAUUGUGUUAGAAAUAGCA |
| | TGAGTTAAAATAAGGTTTAACCTT | CAAUGAGUUAAAAUAAGGUUUAA |
| | AAATGCCAATTTATGTTGGTTTCA | CCUUAAAUGCCAAUUUAUGUUGG |
| | CTTAGG (SEQ ID NO: 301) | UUUCACUUAGG (SEQ ID NO: 400) |
| 2186035 | TATTAAATACTCGTATTGCTGTTTA | UAUUAAAUACUCGUAUUGCUGUU |
| (SEQ ID NO: 58) | AGTATTGTGTTAGAAATAGCACAA | UAAGUAUUGUGUUAGAAAUAGCA |
| | TGAGTTTAAATAAGGTTTAACCTT | CAAUGAGUUUAAAUAAGGUUUAA |
| | AAATGCCAATTTATGTTGGTTTCA | CCUUAAAUGCCAAUUUAUGUUGG |
| | CTTAGGTGAATTTAAGTTCTAGTT | UUUCACUUAGGUGAAUUUAAGUU |
| | ATCTAGAGCTTTT (SEQ ID NO: 302) | CUAGUUAUCUAGAGCUUUU (SEQ |
| | | ID NO: 401) |
| 2186149 | TATTAAATACTCGTATTGCTGTTTT | UAUUAAAUACUCGUAUUGCUGUU |
| (SEQ ID NO: 59) | AGAAATATGTTAGAAATAGCATAT | UUAGAAAUAUGUUAGAAAUAGCA |
| | TGAGTTAAAATAAGTGTAAACTAT | UAUUGAGUUAAAAUAAGUGUAA |
| | AATGCCAAGTAATTGGTTTCACTT | ACUAUAAUGCCAAGUAAUUGGUU |
| | AGGTGA (SEQ ID NO: 303) | UCACUUAGGUGA (SEQ ID NO: 402) |
| 2186149 | TATTAAATACTCGTATTGCTGTTTA | UAUUAAAUACUCGUAUUGCUGUU |
| (SEQ ID NO: 59) | AGAAATATGTTAGAAATAGCATAT | UAAGAAAUAUGUUAGAAAUAGCA |
| | TGAGTTTAAATAAGTGTAAACTAT | UAUUGAGUUUAAAUAAGUGUAA |
| | AATGCCAAGTAATTGGTTTCACTT | ACUAUAAUGCCAAGUAAUUGGUU |
| | AGGTGAAGTTAGAGCTTTTCGAAG | UCACUUAGGUGAAGUUAGAGCUU |
| | CTCTTTT (SEQ ID NO: 304) | UUCGAAGCUCUUUU (SEQ ID NO: |
| | | 403) |
| 2186167 | TATTAAATACTCGTATTGCTGTTTA | UAUUAAAUACUCGUAUUGCUGUU |
| (SEQ ID NO: 60) | AGTACCTGGTAGGAAATTATCAGA | UAAGUACCUGGUAGGAAAUUAUC |
| | TCTACTTAAACAAGGCTTTATGCC | AGAUCUACUUAAACAAGGCUUUA |
| | GAAATCAAGGACATCGACGGATG | UGCCGAAAUCAAGGACAUCGACG |
| | TCC (SEQ ID NO: 305) | GAUGUCC (SEQ ID NO: 404) |
| 2186199 | TATTAAATACTCGTATTGCTGTTTG | UAUUAAAUACUCGUAUUGCUGUU |
| (SEQ ID NO: 61) | AGAGCTTTGTTGAAAAACAAAGC | UGAGAGCUUUGUUGAAAAACAAA |
| | GAGTGCAAATAAGGTTAATCCGA | GCGAGUGCAAAUAAGGUUAAUCC |
| | AATCGCCACTTTGTGGCTCTGCAT | GAAAUCGCCACUUUGUGGCUCUG |
| | GGTGCAGATTTTATTGGTCTCATA | CAUGGUGCAGAUUUUAUUGGUCU |
| | TGAG (SEQ ID NO: 306) | CAUAUGAG (SEQ ID NO: 405) |
| 2186215 | TATTAAATACTCGTATTGCTGTTTG | UAUUAAAUACUCGUAUUGCUGUU |
| (SEQ ID NO: 62) | AGAACCTTGTAGAAATACAAGGT | UGAGAACCUUGUAGAAAUACAAG |
| | GAGTTCAAATAAGGATTTATCCGA | GUGAGUUCAAAUAAGGAUUUAUC |
| | AATTGCATTTTGCCCACATTGTGT | CGAAAUUGCAUUUUGCCCACAUU |
| | GGATTTAAAAAGACCGTTAGGGTC | GUGUGGAUUUAAAAAGACCGUUA |
| | TTT (SEQ ID NO: 307) | GGGUCUUU (SEQ ID NO: 406) |
| 2186264 | TATTAAATACTCGTATTGCTGTTTA | UAUUAAAUACUCGUAUUGCUGUU |
| (SEQ ID NO: 63) | AGATCTATGTCAGAAATGACATAG | UAAGAUCUAUGUCAGAAAUGACA |
| | AGAGTTTAAATAAAGGCTTAGCCC | UAGAGAGUUUAAAUAAAGGCUUA |
| | TAAAAATTTACACTTATGTGGGTG | GCCCUAAAAAUUUACACUUAUGU |
| | GTTGTT (SEQ ID NO: 308) | GGGUGGUUGUU (SEQ ID NO: 407) |
| 2186290 | TATTAAATACTCGTATTGCTGTTTA | UAUUAAAUACUCGUAUUGCUGUU |
| (SEQ ID NO: 64) | GTTACCATATGAGAAATTATATGA | UAGUUACCAUAUGAGAAAUUAUA |
| | CCTAACTAAACAAGGGTTTATCCC | UGACCUAACUAAACAAGGGUUUA |
| | GGATTCGGCTCTTCGGAGCC (SEQ | UCCCGGAUUCGGCUCUUCGGAGC |
| | ID NO: 309) | C (SEQ ID NO: 408) |
| 2186416 | TATTAAATACTCGTATTGCTGTTTA | UAUUAAAUACUCGUAUUGCUGUU |
| (SEQ ID NO: 65) | GCTACCATATGGGAAATTATATGA | UAGCUACCAUAUGGGAAAUUAUA |
| | CCTGGCTAAACAAGGGTTTATCCC | UGACCUGGCUAAACAAGGGUUUA |
| | GGATTCGGCTCCTATAAGGAGCC | UCCCGGAUUCGGCUCCUAUAAGG |
| | (SEQ ID NO: 310) | AGCC (SEQ ID NO: 409) |
| 2186474 | TATTAAATACTCGTATTGCTGTTTT | UAUUAAAUACUCGUAUUGCUGUU |
| (SEQ ID NO: 66) | AGAGTTGTGTTAGAAATAGCACAA | UUAGAGUUGUGUUAGAAAUAGCA |
| | CGAGTTAAAATAAGGTTTAACCTT | CAACGAGUUAAAAUAAGGUUUAA |
| | AAATGTCAATTAAATATTGATTTT | CCUUAAAUGUCAAUUAAAUAUUG |
| | CACTTA (SEQ ID NO: 311) | AUUUUCACUUA (SEQ ID NO: 410) |
| 2186474 | TATTAAATACTCGTATTGCTGTTTA | UAUUAAAUACUCGUAUUGCUGUU |
| (SEQ ID NO: 66) | AGAGTTGTGTTAGAAATAGCACAA | UAAGAGUUGUGUUAGAAAUAGCA |
| | CGAGTTTAAATAAGGTTTAACCTT | CAACGAGUUUAAAUAAGGUUUAA |
| | AAATGTCAATTAAATATTGATTTT | CCUUAAAUGUCAAUUAAAUAUUG |
| | CACTTAGGTGATTTTAAGTTCTAG | AUUUUCACUUAGGUGAUUUUAAG |
| | TTTTCTAGAACTTTT (SEQ ID NO: | UUCUAGUUUUCUAGAACUUUU |
| | 312) | (SEQ ID NO: 411) |
| 2186478 | TATTAAATACTCGTATTGCTGTTTA | UAUUAAAUACUCGUAUUGCUGUU |
| (SEQ ID NO: 67) | AGTTCTATGTCAGAAATGACATAG | UAAGUUCUAUGUCAGAAAUGACA |
| | AGAGTTTAAATAAAGGCTTAGCCC | UAGAGAGUUUAAAUAAAGGCUUA |
| | TAAATGCTCATTAATGTGAGAATG | GCCCUAAAUGCUCAUUAAUGUGA |
| | AACCGT (SEQ ID NO: 313) | GAAUGAACCGU (SEQ ID NO: 412) |
| 2186478 | TATTAAATACTCGTATTGCTGTTTT | UAUUAAAUACUCGUAUUGCUGUU |
| (SEQ ID NO: 67) | AGTTCTATGTCAGAAATGACATAG | UUAGUUCUAUGUCAGAAAUGACA |
| | AGAGTTAAAATAAAGGCTTAGCCC | UAGAGAGUUAAAAUAAAGGCUUA |
| | TAAATGCTCATTAATGTGAGAATG | GCCCUAAAUGCUCAUUAAUGUGA |
| | AACCGTTAGCTGGTTCTTT (SEQ ID | GAAUGAACCGUUAGCUGGUUCUU |
| | NO: 314) | U (SEQ ID NO: 413) |
| 2186573 | TATTAAATACTCGTATTGCTGTTTA | UAUUAAAUACUCGUAUUGCUGUU |
| (SEQ ID NO: 68) | GTTACCATAAAGGAAATTTTATGA | UAGUUACCAUAAAGGAAAUUUUA |
| | CCTAACTAAACAAGGGTTTATCCC | UGACCUAACUAAACAAGGGUUUA |
| | GGATTCGGCTCTTCGGAGCC (SEQ | UCCCGGAUUCGGCUCUUCGGAGC |
| | ID NO: 315) | C (SEQ ID NO: 414) |
| 2186655 | TATTAAATACTCGTATTGCTGTTAC | UAUUAAAUACUCGUAUUGCUGUU |
| (SEQ ID NO: 69) | AGGACCCTGTGAGAAATTACAGGT | ACAGGACCCUGUGAGAAAUUACA |
| | TTCTGTAATAAGGCATAATGCCTT | GGUUUCUGUAAUAAGGCAUAAUG |
| | AGGTTTGATCTAACCTTAAAGATC | CCUUAGGUUUGAUCUAACCUUAA |
| | AT (SEQ ID NO: 316) | AGAUCAU (SEQ ID NO: 415) |
| 2186742 | TATTAAATACTCGTATTGCTGTTAC | UAUUAAAUACUCGUAUUGCUGUU |
| (SEQ ID NO: 70) | AGTACCCTGTGAGAAATTACAGTA | ACAGUACCCUGUGAGAAAUUACA |
| | CCCTGTAGATTTTAAGCAATCTTA | GUACCCUGUAGAUUUUAAGCAAU |
| | AACACGGAACATTTGATTTATTTC | CUUAAACACGGAACAUUUGAUUU |
| | TTTG (SEQ ID NO: 317) | AUUUCUUUG (SEQ ID NO: 416) |
| 2186752 | TATTAAATACTCGTATTGCTATTTA | UAUUAAAUACUCGUAUUGCUAUU |
| (SEQ ID NO: 71) | AGTACCTGTAGAGAAATTTACAGA | UAAGUACCUGUAGAGAAAUUUAC |
| | TCTACTTAAACAAGGCTTTATGCC | AGAUCUACUUAAACAAGGCUUUA |
| | GAACTCAAGGACATCGAAAGATG | UGCCGAACUCAAGGACAUCGAAA |
| | TCC (SEQ ID NO: 318) | GAUGUCC (SEQ ID NO: 417) |
| 2186752 | TATTAAATACTCGTATTGCTATTTT | UAUUAAAUACUCGUAUUGCUAUU |
| (SEQ ID NO: 71) | AGTACCTGAAGAGAAATTTTCAGA | UUAGUACCUGAAGAGAAAUUUUC |
| | TCTACTAAAACAAGGCTTTATGCC | AGAUCUACUAAAACAAGGCUUUA |
| | GAACTCAAGGACATCGAAAGATG | UGCCGAACUCAAGGACAUCGAAA |
| | TCC (SEQ ID NO: 319) | GAUGUCC (SEQ ID NO: 418) |
| 2186833 | TATTAAATACTCGTATTGCTGTTTA | UAUUAAAUACUCGUAUUGCUGUU |
| (SEQ ID NO: 72) | AGTACCCTTAGAGAAATTTAAGGA | UAAGUACCCUUAGAGAAAUUUAA |
| | CCTACTTAAATAAGGCTTTATGCC | GGACCUACUUAAAUAAGGCUUUA |
| | GGATTCAAAGACACCGACGGGTG | UGCCGGAUUCAAAGACACCGACG |
| | TC (SEQ ID NO: 320) | GGUGUC (SEQ ID NO: 419) |
| 2186873 | TATTAAATACTCGTATTGCTGTTTG | UAUUAAAUACUCGUAUUGCUGUU |
| (SEQ ID NO: 73) | AGAACCTTGTAGAAAACAAGTGT | UGAGAACCUUGUAGAAAACAAGU |
| | GAGTGCAAATAAGGATTTTCCGAA | GUGAGUGCAAAUAAGGAUUUUCC |
| | ATCGCCGAAAGGCCTGCATGGTGC | GAAAUCGCCGAAAGGCCUGCAUG |
| | AGATTAAAAGACTTGACTTTTCAA | GUGCAGAUUAAAAGACUUGACUU |
| | GTC (SEQ ID NO: 321) | UUCAAGUC (SEQ ID NO: 420) |
| 2186985 | TATTAAATACTCGTATTGCTATTTA | UAUUAAAUACUCGUAUUGCUAUU |
| (SEQ ID NO: 74) | AGTACCTGTAAAGAAATTTACAGA | UAAGUACCUGUAAAGAAAUUUAC |
| | TCTGCTTAAACAAGGCTTTATGCC | AGAUCUGCUUAAACAAGGCUUUA |
| | GAAATCAAGGACATCGACGGATG | UGCCGAAAUCAAGGACAUCGACG |
| | TCC (SEQ ID NO: 322) | GAUGUCC (SEQ ID NO: 421) |
| 2187023 | TATTAAATACTCGTATTGCTGTTTT | UAUUAAAUACUCGUAUUGCUGUU |
| (SEQ ID NO: 75) | AGATTTATGTTAGAAATAGCATAA | UUAGAUUUAUGUUAGAAAUAGCA |
| | ATGAGTTAAAATAAGGTAAAACCT | UAAAUGAGUUAAAAUAAGGUAA |
| | TAAATGCCAATTTAATATTGGTTTT | AACCUUAAAUGCCAAUUUAAUAU |
| | GCTTA (SEQ ID NO: 323) | UGGUUUUGCUUA (SEQ ID NO: 422) |
| 2187023 | TATTAAATACTCGTATTGCTGTTTA | UAUUAAAUACUCGUAUUGCUGUU |
| (SEQ ID NO: 75) | AGATTTATGTTAGAAATAGCATAA | UAAGAUUUAUGUUAGAAAUAGCA |
| | ATGAGTTTAAATAAGGTAAAACCT | UAAAUGAGUUUAAAUAAGGUAA |
| | TAAATGCCAATTTAATATTGGTTTT | AACCUUAAAUGCCAAUUUAAUAU |
| | GCTTAGGCAAGTAAAGTCTTAGTT | UGGUUUUGCUUAGGCAAGUAAAG |
| | ATCTAAGGCTTTT (SEQ ID NO: 324) | UCUUAGUUAUCUAAGGCUUUU |
| | | (SEQ ID NO: 423) |
| 2187103 | TATTAAATACTCGTATTGCTGTTTG | UAUUAAAUACUCGUAUUGCUGUU |
| (SEQ ID NO: 76) | AGAACCTTGTAGAAATGCAAGGT | UGAGAACCUUGUAGAAAUGCAAG |
| | GAGTTCAAATAAGGATTTATCCGA | GUGAGUUCAAAUAAGGAUUUAUC |
| | AATCGCCGTACGGCCCACATTGTG | CGAAAUCGCCGUACGGCCCACAU |
| | TGGATGAAAAGACTTGGTTTCCAG | UGUGUGGAUGAAAAGACUUGGUU |
| | GTC (SEQ ID NO: 325) | UCCAGGUC (SEQ ID NO: 424) |
| 2187334 | TATTAAATACTCGTATTGCTGTTTA | UAUUAAAUACUCGUAUUGCUGUU |
| (SEQ ID NO: 77) | AGATCTATGTCAGAAATGACATAG | |
| | AGAGTTTAAATAAAGCTTTAAGCC | |
| | TAAATGCTCACTTATGTGTTCTGA | UAAGAUCUAUGUCAGAAAUGACA |
| | ACCGTT (SEQ ID NO: 326) | UAGAGAGUUUAAAUAAAGCUUUA |
| | | AGCCUAAAUGCUCACUUAUGUGU |
| | | UCUGAACCGUU (SEQ ID NO: 425) |

TABLE 7 provides exemplary disease and syndromes for compositions, systems and methods described herein.

TABLE 7
Diseases and Syndromes
Exemplary Diseases and Syndromes

11-hydroxylase deficiency; 17,20-desmolase deficiency; 17-hydroxylase deficiency; 3-
hydroxyisobutyrate aciduria; 3-hydroxysteroid dehydrogenase deficiency; 46,XY gonadal dysgenesis;
AAA syndrome; ABCA3 deficiency; ABCC8-associated hyperinsulinism; aceruloplasminemia;
acromegaly; achondrogenesis type 2; acral peeling skin syndrome; acrodermatitis enteropathica;
adrenocortical micronodular hyperplasia; adrenoleukodystrophies; adrenomyeloneuropathies; Aicardi-
Goutieres syndrome; Alagille disease (also called Alagille Syndrome); Alexander Disease; Alpers
syndrome; alpha-1 antitrypsin deficiency (AATD); alpha-mannosidosis; Alstrom syndrome; Alzheimer's
disease; amebic dysentery; amelogenesis imperfecta; amish type microcephaly; amyotrophic lateral
sclerosis (ALS); anaplastic large cell lymphoma; anauxetic dysplasia; androgen insensitivity syndrome;
angiopathic thrombosis; antiphospholipid syndrome; Antley-Bixler syndrome; APECED; Apert
syndrome; aplasia of lacrimal and salivary glands; arginase-1 deficiency; argininosuccinic aciduria;
argininemia; arrhythmogenic right ventricular dysplasia; Arts syndrome; ARVD2; arylsulfatase
deficiency type metachromatic leukodystrophy; ataxia telangiectasia; atherosclerotic cardiovascular
disease; autoimmune lymphoproliferative syndrome; autoimmune polyglandular syndrome type 1;
autosomal dominant anhidrotic ectodermal dysplasia; autosomal dominant deafness; autosomal
dominant polycystic kidney disease; autosomal recessive microtia; autosomal recessive renal glucosuria;
autosomal visceral heterotaxy; babesiosis; balantidial dysentery; Bardet-Biedl syndrome; Bartter
syndrome; basal cell nevus syndrome; Batten disease; benign recurrent intrahepatic cholestasis; beta-
mannosidosis; B-thalassemia; Bethlem myopathy; Blackfan-Diamond anemia; bleeding disorder
(coagulation); blepharophimosis; Byler disease; C syndrome; CADASIL; calcific aortic stenosis;
calcification of joints and arteries; carbamoyl phosphate synthetase I deficiency; cardiofaciocutaneous
syndrome; Carney triad; carnitine palmitoyltransferase deficiencies; cartilage-hair hypoplasia; cblC type
of combined methylmalonic aciduria; CD18 deficiency;
CD3Z-associated primary T-cell
immunodeficiency; CD40L deficiency; CDAGS syndrome; CDGIA; CDGIB; CDGIM; CDG2C;
CEDNIK syndrome; central core disease; centronuclear myopathy; cerebral capillary malformation;
cerebrooculofacioskeletal syndrome type 4; cerebrooculogacioskeletal syndrome; cerebrotendinous
xanthomatosis; Chaga's Disease; Charcot Marie Tooth Disesase; cherubism; CHILD syndrome; chronic
granulomatous disease; chronic recurrent multifocal osteomyelitis; cirrhosis; citrin deficiency;
citrullinemia type I; citrullinemia type II; classic hemochromatosis; CNPPB syndrome; cobalamin C
disease; Cockayne syndrome; coenzyme Q10 deficiency; Coffin-Lowry syndrome; Cohen syndrome;
combined deficiency of coagulation factors V; common variable immune deficiency 3; complement
hyperactivation; complete androgen insentivity; cone rod dystrophies; conformational diseases;
congenital adrenal hyperplasia; congenital bile adid synthesis defect type 1; congenital bile adid synthesis
defect type 2; congenital defect in bile acid synthesis type; congenital erythropoietic porphyria;
congenital generalized osteosclerosis; congenital hyperplasia (CAH); congenital muscular dystrophy 1A
(MDCIA); Cornelia de Lange syndrome; coronary heart disease; Cousin syndrome; Cowden disease;
COX deficiency; Cri du chat syndrome; Crigler-Najjar disease; Crigler-Najjar syndrome type 1; Crisponi
syndrome; Crouzon syndrome; Currarino syndrome; Curth-Macklin type ichthyosis hystrix; cutis laxa;
cystic fibrosis; cystinosis; d-2-hydroxyglutaric aciduria; DDP syndrome; Dejerine-Sottas disease;
Denys-Drash syndrome; Dercum disease; desmin cardiomyopathy; desmin myopathy; DGUOK-
associated mitochondrial DNA depletion; diabetes Type I; diabetes Type II; disorders of glutamate
metabolism; distal spinal muscular atrophy type 5; DNA repair diseases; dominant optic atrophy; Doyne
honeycomb retinal dystrophy; Dravet Syndrome; Duchenne muscular dystrophy; dyskeratosis congenita;
Ehlers-Danlos syndrome type 4; Ehlers-Danlos syndromes; Elejalde disease; Ellis-van Creveld disease;
Emery-Dreifuss muscular dystrophies; encephalomyopathic mtDNA depletion syndrome; encephalitis;
enzymatic diseases; EPCAM-associated congenital tufting enteropathy; epidermolysis bullosa with
pyloric atresia; epilepsy; fabry disease; facioscapulohumeral muscular dystrophy; Factor V Leiden
thrombophilia; Faisalabad histiocytosis; familial atypical mycobacteriosis; familial capillary
malformation-arteriovenous; Familial Creutzfeld-Jakob disease; familial esophageal achalasia; familial
glomuvenous malformation; familial hemophagocytic lymphohistiocytosis; familial mediterranean
fever; familial megacalyces; familial schwannomatosis; familial spina bifida; familial splenic
asplenia/hypoplasia; familial thrombotic thrombocytopenia purpura; Fanconi disease (Fanconi anemia);
Feingold syndrome; FENIB; fibrodysplasia ossificans progressiva; FKTN; Fragile X syndrome;
Francois-Neetens fleck corneal dystrophy; Frasier syndrome; Friedreich's ataxia; FTDP-17; Fuchs
corneal dystrophy; fucosidosis; G6PD deficiency; galactosialidosis; Galloway syndrome; Gardner
syndrome; Gaucher disease; Gitelman syndrome; GLUT1 deficiency; GM2- Gangliosidoses (e.g., Tay
Sachs Disease, Sandhoff Disease) glycogen storage disease type 1b; glycogen storage disease type 2;
glycogen storage disease type 3; glycogen storage disease type 4; glycogen storage disease type 9a;
glycogen storage diseases; GM1-gangliosidosis; Greenberg syndrome; Greig cephalopolysyndactyly
syndrome; hair genetic diseases; hairy cell leukemia; HANAC syndrome; harlequin type ichtyosis
congenita; HDR syndrome; hearing loss; hemochromatosis type 3; hemochromatosis type 4; hemolytic
anemia; hemolytic uremic syndrome; hemophilia A; hemophilia B; hereditary angioedema type 3;
hereditary angioedemas; hereditary hemorrhagic telangiectasia; hereditary hypofibrinogenemia;
hereditary intraosseous vascular malformation; hereditary leiomyomatosis and renal cell cancer;
hereditary neuralgic amyotrophy; hereditary sensory and autonomic neuropathy type; Hermansky-Pudlak
disease; HHH syndrome; HHT2; hidrotic ectodermal dysplasia type 1; hidrotic ectodermal dysplasias;
histiocytic sarcoma; HNF4A-associated hyperinsulinism; HNPCC; homozygous familial
hypercholesterolemia; human immunodeficiency with microcephaly; Human monkeypox (MPX);
human papilloma virus (HPV) infection; Huntington's disease; hyper-IgD syndrome; hyperinsulinism-
hyperammonemia syndrome; hypercholesterolemia; hypertrophy of the retinal pigment epithelium;
hypochondrogenesis; hypohidrotic ectodermal dysplasia; ICF syndrome; idiopathic congenital intestinal
pseudo-obstruction;
immunodeficiency
13; immunodeficiency 17; immunodeficiency 25;
immunodeficiency with hyper-IgM type 1; immunodeficiency with hyper-IgM type 3;
immunodeficiency with hyper-IgM type 4; immunodeficiency with hyper-IgM type 5; immunoglobulin
alpha deficiency; inborn errors of thyroid metabolism; infantile myofibromatosis; infantile visceral
myopathy; infantile X-linked spinal muscular atrophy; intrahepatic cholestasis of pregnancy; IPEX
syndrome; IRAK4 deficiency; isolated congenital asplenia; Jeune syndrome; Johanson-Blizzard
syndrome; Joubert syndrome; JP-HHT syndrome; juvenile hemochromatosis; juvenile hyalin
fibromatosis; juvenile nephronophthisis; Kabuki mask syndrome; Kallmann syndromes; Kartagener
syndrome; KCNJ11-associated hyperinsulinism; Kearns-Sayre syndrome; Kostmann disease; Kozlowski
type of spondylometaphyseal dysplasia; Krabbe disease; LADD syndrome; late infantile-onset neuronal
ceroid lipofuscinosis; LCK deficiency; LDHCP syndrome; Leber Congenital Amaurosis Teyp 10; Legius
syndrome; Leigh syndrome; lethal congenital contracture syndrome 2; lethal congenital contracture
syndromes; lethal contractural syndrome type 3; lethal neonatal CPT deficiency type 2; lethal
osteosclerotic bone dysplasia; leukocyte adhesion deficiency; Li Fraumeni syndrome; LIG4 syndrome;
limb girdle muscular dystrophies (LGMD1B, LGMD2A, LGMD2B); lipodystrophy; lissencephaly type
1; lissencephaly type 3; Loeys-Dietz syndrome; low phospholipid-associated cholelithiasis; Lynch
Syndrome; lysinuric protein intolerance; a lysosomal storage disease (e.g., Hunter syndrome, Hurler
syndrome); macular dystrophy; Maffucci syndrome; Majeed syndrome; mannose-binding protein
deficiency; mantle cell lymphoma; Marfan disease; Marshall syndrome; MASA syndrome; mastocytosis;
MCAD deficiency; McCune-Albright syndrome; MCKD2; Meckel syndrome; MECP2 Duplication
Syndrome; Meesmann corneal dystrophy; megacystis-microcolon-intestinal hypoperistalsis;
megaloblastic anemia type 1; MEHMO; MELAS; Melnick-Needles syndrome; MEN2s; meningitis;
Menkes disease; metachromatic leukodystrophies; methymalonic acidemia due to transcobalamin
receptor defect; methylmalonic acidurias; methylvalonic aciduria; microcoria-congenital nephrosis
syndrome; microvillous atrophy; migraine; mitochondrial neurogastrointestinal encephalomyopathy;
monilethrix; monosomy X; mosaic trisomy 9 syndrome; Mowat-Wilson syndrome; mucolipidosis type
2; mucolipidosis type Ma; mucolipidosis type IV; mucopolysaccharidoses; mucopolysaccharidosis type
3A; mucopolysaccharidosis type 3C; mucopolysaccharidosis type 4B; multiminicore disease; multiple
acyl-CoA dehydrogenation deficiency; multiple cutaneous and mucosal venous malformations; multiple
endocrine neoplasia type 1; multiple sulfatase deficiency; mycosis fungoides; myotonic dystrophy;
NAIC; nail-patella syndrome; nemaline myopathies; neonatal diabetes mellitus; neonatal surfactant
deficiency; nephronophtisis; Netherton disease; neurofibromatoses; neurofibromatosis type 1; Niemann-
Pick disease type A; Niemann-Pick disease type B; Niemann-Pick disease type C; NKX2E; non-alcoholic
fatty liver disease (NAFLD); non-alcoholic steatohepatitis (NASH); Noonan syndrome; North American
Indian childhood cirrhosis; NROB1 duplication-associated DSD; ocular genetic diseases; oculo-auricular
syndrome; OLEDAID; oligomeganephronia; oligomeganephronic renal hypolasia; Ollier disease; Opitz-
Kaveggia syndrome; ornithine transcarbamylase deficiency (OTCD); orofaciodigital syndrome type 1;
orofaciodigital syndrome type 2; osseous Paget disease; osteogenesis imperfecta; otopalatodigital
syndrome type 2; OXPHOS diseases; palmoplantar hyperkeratosis; panlobar nephroblastomatosis;
Parkes-Weber syndrome; Parkinson's disease; partial deletion of 21q22.2-q22.3; Pearson syndrome;
Pelizaeus-Merzbacher disease; Pendred syndrome; pentalogy of Cantrell; peroxisomal acyl-CoA-oxidase
deficiency; Peutz-Jeghers syndrome; Pfeiffer syndrome; Pierson syndrome; pigmented nodular
adrenocortical disease; pipecolic acidemia; Pitt-Hopkins syndrome; plasmalogens deficiency; platelet
glycoprotein IV deficiency; pleuropulmonary blastoma and cystic nephroma; polycystic kidney disease;
polycystic ovarian disease; polycystic lipomembranous osteodysplasia; Pompe disease; including
infantile onset Pompe disease (IOPD) and late onset Pompe disease (LOPD); porphyrias; PRKAG2
cardiac syndrome; premature ovarian failure; primary erythermalgia; primary hemochromatoses; primary
hyperoxaluria; progressive familial intrahepatic cholestasis; propionic acidemia; protein-losing
enteropathy; pyruvate decarboxylase deficiency; RAPADILINO syndrome; renal cystinosis; retinitis
pigmentosa; Rett Syndrome; rhabdoid tumor predisposition syndrome; Rieger syndrome; ring
chromosome 4; Roberts syndrome; Robinow-Sorauf syndrome; Rothmund-Thomson syndrome; severe
combined immunodeficiency disorder (SCID); Saethre-Chotzen syndrome; Sandhoff disease; SC
phocomelia syndrome; SCAS; Schinzel phocomelia syndrome; severe hypertriglyceridemia; short rib-
polydactyly syndrome type 1; short rib-polydactyly syndrome type 4; short-rib polydactyly syndrome
type 2; short-rib polydactyly syndrome type 3; Shwachman disease; Shwachman-Diamond disease;
sickle cell anemia; Silver-Russell syndrome; Simpson-Golabi-Behmel syndrome; Smith-Lemli-Opitz
syndrome; SPG7-associated hereditary spastic paraplegia; spherocytosis; spinocerebellar ataxia; spinal
muscular atrophy; split-hand/foot malformation with long bone deficiencies; spondylocostal dysostosis;
sporadic amyotrophic lateral sclerosis; sporadic visceral myopathy with inclusion bodies; storage
diseases; Stargardt macular dystrophy; STRA6-associated syndrome; stroke; Tay-Sachs disease;
thanatophoric dysplasia; thrombophilia due to antithrombin III deficiency; thyroid metabolism diseases;
Tourette syndrome; transcarbamylase deficiency; transthyretin-associated amyloidosis; trisomy 13;
trisomy 22; trisomy 2p syndrome; tuberous sclerosis; tufting enteropathy; ullrich congenital muscular
dystrophy (UCMD); urea cycle diseases; Usher Syndrome; Van Den Ende-Gupta syndrome; Van der
Woude syndrome; variegated mosaic aneuploidy syndrome; VLCAD deficiency; von Hippel-Lindau
disease; von Willebrand disease; Waardenburg syndrome; WAGR syndrome; Walker-Warburg
syndrome; Werner syndrome; Wilson's disease; Wiskott-Aldrich Syndrome; Wolcott-Rallison
syndrome; Wolfram syndrome; X-linked agammaglobulinemia; X-linked chronic idiopathic intestinal
pseudo-obstruction; X-linked cleft palate with ankyloglossia; X-linked dominant chondrodysplasia
punctata; X-linked ectodermal dysplasia; X-linked Emery-Dreifuss muscular dystrophy; X-linked
lissencephaly; X-linked lymphoproliferative disease; X-linked visceral heterotaxy; xanthinuria type 1;
xanthinuria type 2; xeroderma pigmentosum; XPV; and Zellweger disease.

TABLE 8 provides exemplary target nucleic acids that are useful in the compositions, systems and methods described herein.

TABLE 8
Exemplary Target Nucleic Acids
Exemplary target genes

AAVS1, ABCA4, ABCB11, ABCC8, ABCDI, ABCG5, ABCG8, ACAD9, ACADM, ACADVL, ACAT1,
ACTA1, ACOX1, ACSF3, ADA, ADAMTS2, ADGRGI, AGA, AGL, AGPS, AGXT, AHI1, AIRE,
ALDH3A2, ALDOB, ALG6, ALK, ALKBH5, ALMS1, ALPL, AMRC9, AMT, ANAPC10, ANAPC11,
ANGPTL3, ANGPTL4, APC, Apo(a), APOCIII, APOE&4, APOL1, APP, AQP2, AR, ARFRP1, ARG1,
ARH, ARL13B, ARL6, ARSA, ARSB, ASL, ASNS, ASPA, ASSI, ATM, ATP6V1B1, ATP7A, ATP7B, ATRX,
ATXN1, ATXN10, ATXN2, ATXN3, ATXN7, ATXN8OS, AXIN1, AXIN2, B2M, BACE-1, BAK1, BAP1,
BARD1, BAX2, BBS1, BBS10, BBS12, BBS2, BCKDHA, BCKDHB, BCL2L2, BCS1L, BEST1, Betaglobin
gene, BLM, BMPRIA, BRAF, BRAFV600E, BRCA1, BRCA2, BRIP1, BSND, C9orf72, CA4, CACNAIA,
CAH1, CAPN3, CASR, CBS, CCNB1 CC2D2A, CCR5, CD1, CD2, CD3, CD3D, CD3Z, CD4, CD5,
CD6, CD7, CD8A, CD8B, CD9, CD14, CD18, CD19, CD21, CD22, CD23, CD27, CD28, CD30, CD33,
CD34, CD36, CD38, CD40, CD40L, CD44, CD46, CD47, CD48, CD52, CD55, CD57, CD58, CD59,
CD68, CD69, CD72, CD73, CD74, CD79A, CD80, CD81, CD83, CD84, CD86, CD90, CD93, CD96,
CD99, CD100, CD123, CD160, CD163, CD164, CD164L2, CD166, CD200, CD204, CD207, CD209,
CD226, CD244, CD247, CD274, CD276, CD300, CD320, CDC73, CDH1, CDH23, CDK11, CDK4,
CDKN1A, CDKN1B, CDKN1C, CDKN2A, CDKN2B, CEBPA, CELA3B, CEP 290, CERKL, CFB, CFTR,
CHCHD10, CHEK2, CHM, CHRNE, CIDEB, CIITA, CLN3, CLN5, CLN6, CLN8, CLRN1, CLTA,
CMT1A, CNBP, CNGB1, CNGB3, COL1A1, COL1A2, COL27A1, COL4A3, COL4A4, COL4A5,
COL6A1, COL6A2, COL6A3, COL7A1, CPS1, CPT1A, CPT2, CRB1, CREBBP, CRX, CRYAA,
CTNNA1, CTNNB1, CTNND2, CTNS, CTSK, CXCL12, CYBA, CYBB, CYP11B1, CYP11B2, CYP17A1,
CYP19A1, CYP21A2, CYP27A1, DBT, DCC, DCLRE1C, DERL2, DFNA36, DFNB31, DGAT2, DHCR7,
DHDDS, DICER1, DIS3L2, DLD, DMD, DMPK, DNAH5, DNAI1, DNAI2, DNM2, DNMT1, DPC4,
DYSF, EDA, EDN3, EDNRB, EGFR, EIF2B5, EMC2, EMC3, EMD, EMX1, EN1, EPCAM, ERCC6,
ERCC8, ESCO2, ETFA, ETFDH, ETHE1, EVC, EVC2, EYS, F5, F9, FX1, FAH, FAM161A, FANCA,
FANCB, FANCC, FANCD1, FANCD2, FANCE, FANCF, FANCG, FANC1, FANCJ, FANCL, FANCM,
FANCN, FANCP, FANCS, FBN1, FGF14, FGFR2, FGFR3, FGA, FGB, FGG, FH, FHL1, FIX, FKRP,
FKTN, FLCN, FMR1, FOXP3, FSCN2, FSHD1, FUS, FUT8, FVIII, FXII, FXN, G6PC, GAA, GALC,
GALK1, GALT, GAMT, GATA2, GATA-4, GBA, GBE1, GCDH, GCGR, GDNF, GFAP, GFM1, GHR,
GJB1, GJB2, GLA, GLB1, GLDC, GLE1, GNE, GNPTAB, GNPTG, GNS, GPAM, GPC3, GPR98,
GREM1, GRHPR, GRIN2B, H2AFX, H2AX, HADHA, HAX1, HBA1, HBA2, HBB, HBV cccDNA, HER2,
HEXA, HEXB, HFE, HGSNAT, HLCS, HMGCL, HAO1, HOGA1, HOXB13, HPRPF3, HPRT1, HPS1,
HPS3, HRAS, HRD1, HSD3B2, HSD17B4, HSD17B13, HTT, HUS1, HYAL1, HYLS1, IDS, IDUA,
IFITM5, IKBKAP, IL2RG, IL7R, IMPDH1, INPP5E, IRF4, ITGB2, ITPR1, IVD, JAG1, JAK1, JAK3,
KCNC3, KCND3, KCNJ11, KLKB1, KLHL7, KRAS, LAMA1, LAMA2, LAMA3, LAMB3, LAMC2, LCA5,
LDHA, LDLR, LDLRAP1, LHX3, LIFR, LIPA, LMNA, LMOD3, LOR, LOXHD1, LPA, LPL, LRAT,
LRP6, LRPPRC, LRRK2, MADR2, MAN2B1, MAPT, MARC1, MAX, MCM6, MCOLN1, MECP2,
MED17, MEFV, MEN1, MERTK, MESP2, MET, METex14, MFN2, MFSD8, MIA3, MITF, MKL2,
MKS1, MLC1, MLH1, MLH3, MMAA, MMAB, MMACHC, MMADHC, MMD, MPI, MPL, MPV17,
MSH2, MSH3, MSH6, MTHFD1L, MTHFR, MTM1, MTRR, MTTP, MUT, MUTYH, MYC, MYH7,
MYO7A, NAGLU, NAGS, NBN, NDRG1, NDUFAF5, NDUFS6, NEB, NF1, NF2, NKX2-5, NOG,
NOTCH1, NOTCH2, NPC1, NPC2, NPHP1, NPHS1, NPHS2, NRAS, NR2E3, NTHL1, NTRK, NTRK1,
OAT, OCT4, OFD1, OPA3, OTC, PAH, PALB2, PAQR8, PAX3, PC, PCCA, PCCB, PCDH15, PCSK9,
PD1, PDCD1, PDE6B, PDGFRA, PDHA1, PDHB, PEX1, PEX10, PEX12, PEX13, PEX14, PEX16,
PEX19, PEX2, PEX26, PEX3, PEX5, PEX6, PEX7, PFKM, PHGDH, PHOX2B, PKD1, PKD2, PKHD1,
PKK, PLEKHG4, PMM2, PMP22, PMS1, PMS2, PNPLA3, POLD1, POLE, POMGNT1, POT1,
POU5F1, PPM1A, PPP2R2B, PPT1, PRCD, PRKAG2, PRKAR1A, PRKCG, PRNP, PROM1, PROP1,
PRPF31, PRPF8, PRPH2, PRPS1, PSAP, PSD3, PSD95, PSEN1, PSEN2, PSRC1, PTCH1, PTEN, PTS,
PUS1, PYGM, RAB23, RAD50, RAD51C, RAD51D, RAG1, RAG2, RAPSN, RARS2, RB1, RDH12,
RECQL4, RET, RHO, RICTOR, RMRP, ROS1, RP1, RP2, RPE65, RPGR, RPGRIP1L, RPL32P3, RS1,
RTCA, RTEL1, RUNX1, SACS, SAMHD1, SCN1A, SCN2A, SDHA, SDHAF2, SDHB, SDHC, SDHD,
SEL1L, SEPSECS, SERPINA1, SERPINC1, SERPING1, SGCA, SGCB, SGCG, SGSH, SIRT1, SLC12A3,
SLC12A6, SLC17A5, SLC22A5, SLC25A13, SLC25A15, SLC26A2, SLC26A4, SLC35A3, SLC35B4,
SLC37A4, SLC39A4, SLC4A11, SLC6A8, SLC7A7, SMAD3, SMAD4, SMARCA4, SMARCAL1,
SMARCB1, SMARCE1, SMN1, SMPD1, SNAI2, SNCA, SNRNP200, SOD1, SOX10, SPARA7, SPTBN2,
STAR, STAT3, STK11, SUFU, SUMF1, SYNE1, SYNE2, SYS1, TARDBP, TAT, TBK1, TBP, TCIRG1,
TCTN3, TECPR2, TERC, TERT, TFR2, TGFBR2, TGM1, TH, TLE3, TMEM127, TMEM138, TMEM216,
TMEM43, TMEM67, TMPRSS6, TNNI2, TNNT1, TNNT3, TOP1, TOPORS, TP53, TPM2, TPM3, TPP1,
TRAC, TRMU, TSC1, TSC2, TSFM, TSPAN14, TTBK2, TTC8, TTPA, TTR, TULP1, TYMP, UBE2G2,
UBE2J1, UBE3A, USH1C, USH1G, USH2A, VEGF, VHL, VPS13A, VPS13B, VPS35, VPS45, VRK1,
VSX2, VWF, WAS, WDR19, WDR48, WNT10A, WRN, WS2B, WS2C, WT1, XPA, XPC, XPF, XRCC3,
YAP1, ZAC1, ZEB1, ZFYVE26, and ZNF423

### EXAMPLES

The following examples are included for illustrative purposes only and are not intended to limit the scope of the invention.

### Example 1. PAM Screening for Effector Proteins

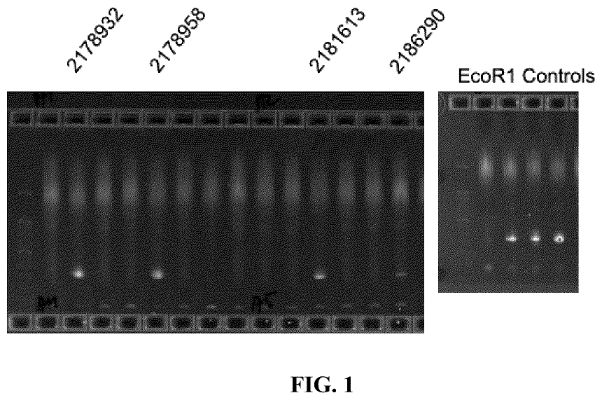

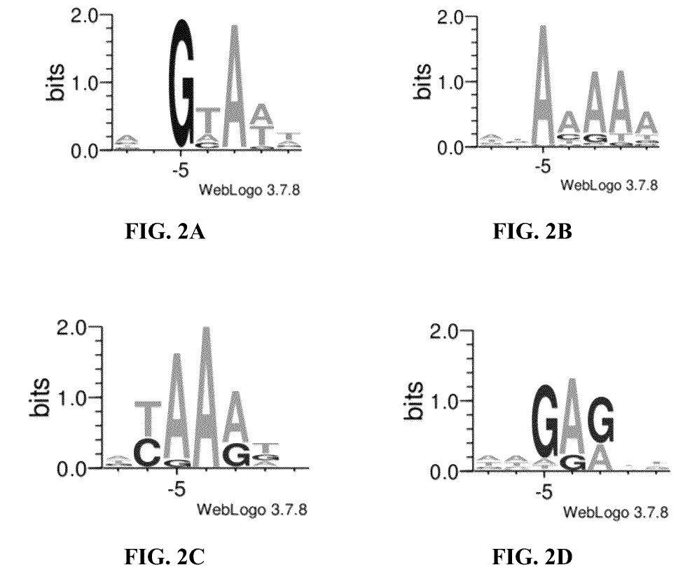

To assess the ability of these protein to carry out cis-cleavage, an in vitro enrichment experiment was carried out. Effector proteins and guide RNA combinations shown in TABLE 9 below were screened by an in vitro enrichment (IVE) assay to determine PAM recognition by each effector protein-guide RNA complex. Briefly, effector proteins were complexed with corresponding guide RNAs for 15 minutes at 37° C. The complexes were added to an IVE reaction mix. PAM screening reactions used 10 μl of RNP in 100 μl reactions with 1,000 ng of a plasmid library containing a randomized PAM sequence 3′ of a target protospacer (5′-NNNNNNN-3′, where N is any of A, C, G, T) in 1× Cutsmart buffer and were carried out for 15 minutes at 25° C., 45 minutes at 37° C., and 15 minutes at 45° C. Reactions were terminated with 1 μl of proteinase K and 5 μl of 500 mM EDTA for 30 minutes at 37° C. Any target plasmid that was successfully cleaved had an adapter ligated to the cut end, enabling PCR amplification. Bands on the gel shown in FIG. 1 show effector protein-sgRNA complexes that were capable of cis-cleavage. An EcoRI site was included near the spacer and this was used as a positive control. Next generation sequencing (NGS) was performed on cut sequences to identify enriched PAMs. FIGS. 2A-2D show the consensus PAM sequences associated with each effector/guide complex indicated in TABLE 9. Consensus sequences are shown in TABLE 9.

TABLE 9
Effector Protein Guide Complexes

| Effector | | | |
|---|---|---|---|
| Protein | Guide Nucleic | | |
| Name | Acid Name | Consensus | |
| (SEQ ID | (SEQ ID NO | 3' PAM | |
| NO) | for DNA/RNA) | sequence | Web Logo |
| 2178932 | R7766 (SEQ ID | NNGTAW | FIG. 2A |
| (SEQ ID | NO: 234/ | (SEQ ID | |
| NO: 6) | SEQ ID NO: 333) | NO: 119) | |
| 2178958 | R7775 (SEQ ID | NNAMRWA | FIG. 2B |
| (SEQ ID | NO: 235/ | (SEQ ID | |
| NO: 7) | SEQ ID NO: 334) | NO: 120) | |
| 2181613 | R7903 (SEQ ID | NYAAR | FIG. 2C |
| (SEQ ID | NO: 259/ | (SEQ ID | |
| NO: 25) | SEQ ID NO: 358) | NO: 121) | |
| 2186290 | R7790 (SEQ ID | NNKRR | FIG. 2D |
| (SEQ ID | NO: 309/ | (SEQ ID | |
| NO: 64) | SEQ ID NO: 408) | NO: 122) | |

### Example 2. Detecting Target Nucleic Acids

Effector proteins are tested for trans cleavage. Briefly, partially purified (e.g., nickel-NTA purified) effector proteins are incubated with crRNA and tracrRNA sequence (or an sgRNA) in a trans cleavage buffer (e.g., 20 mM Tricine, 15 mM MgCl₂, 0.2 mg/ml BSA, 1 mM TCEP (pH 9 at 37° C.)) at room temperature for about 10 to about 30 minutes, followed by addition of a target nucleic acid to produce effector-protein guide complexes. Trans cleavage activity is detected by fluorescence signal upon cleavage of a fluorophore-quencher reporter. Dilutions of the effector-protein guide complexes are performed, and the assay repeated at various concentrations of the effector-protein guide complexes.

### Example 3. Effector Proteins Edit Genomic DNA in Mammalian Cells

Effector proteins are tested for their ability to produce indels in a mammalian cell line (e.g., HEK293T cells). Briefly, a plasmid encoding the effector proteins and a guide RNA are delivered by lipofection to the mammalian cells. This is performed with a variety of guide RNAs targeting several loci adjacent to biochemically determined PAM sequences. Indels in the loci are detected by next generation sequencing of PCR amplicons at the targeted loci and indel percentage is calculated as the fraction of sequencing reads containing insertions or deletions relative to an unedited reference sequence. “No plasmid” and Cas9 are included as negative and positive controls, respectively.

### Example 4. Base Editing

A nucleic acid vector encoding a fusion protein is constructed for base editing. The fusion protein comprises a catalytically inactive variant of an effector protein fused to a deaminase. The fusion protein and at least one guide nucleic acid is tested for its ability to edit a target sequence in eukaryotic cells. Cells are transfected with the nucleic acid vector and guide nucleic acid. After sufficient incubation, DNA is extracted from the transfected cells. Target sequences are PCR amplified and sequenced by NGS and MiSeq. The presence of base modifications are analyzed from sequencing data. Results are recorded as a change in % base call relative to the negative control.

### Example 5. Activation of Gene Expression with Cas Effector Fusion Polypeptide

A single stranded reporter nucleic acid encoding a fluorescent protein (e.g., enhanced green fluorescent protein (EGFP)) and a eukaryotic promoter is generated with a target sequence that is known to be recognized by complexes of effector proteins disclosed herein and corresponding guide nucleic acids. A nucleic acid vector encoding the Cas effector fused to a transcriptional activator; a guide nucleic acid; and the single stranded reporter nucleic acid encoding EGFP are introduced to eukaryotic cells via lipofection and EGFP expression is quantified by flow cytometry. Relative amounts of RNA, indicative of relative gene expression, are quantified with RT-qPCR.

### Example 6. Reduction of Gene Expression with Cas Effector Fusion Polypeptide

A single stranded reporter nucleic acid encoding a fluorescent protein (e.g., enhanced green fluorescent protein (EGFP)) and a pSV40 promoter that drives constitutive expression of EGFP is generated with a target sequence that is known to be recognized by complexes of effector proteins disclosed herein and corresponding guide nucleic acids. A nucleic acid vector encoding the Cas effector fused to a transcriptional repressor; a guide nucleic acid; and the single stranded reporter nucleic acid encoding EGFP are introduced to eukaryotic cells via lipofection and EGFP expression is quantified by flow cytometry. Relative amounts of RNA, indicative of relative gene expression, are quantified with RT-qPCR.

### Example 7. Generating a Catalytically Inactive Variant of a CRISPR Cas Effector Protein

Extensive work has been done to evaluate the overall domain structure of the CRISPR Cas enzymes in the last decade. These data can be an effective reference when trying to identify a catalytic residue of a Cas nuclease. By selecting the residue of a Cas nuclease of interest that aligns at the same relative location as the catalytic residue of a known nuclease when the Cas nuclease and known nuclease are aligned for maximal sequence identity, one can identify the catalytic residue of the Cas nuclease.

Sequence or structural analogs of a Cas nuclease provide an additional or supplemental way to predict the catalytic residues of the novel Cas nuclease relative to the previous description in this Example. Catalytic residues are usually highly conserved and can be identified in this manner.

Alternatively, or additionally to the description already provided in this Example, computational software may be used to predict the structure of a Cas nuclease.

### Example 8. Additional PAM Screening for Effector Proteins

To assess the ability of these protein to carry out cis-cleavage, an in vitro enrichment experiment was carried out. Effector proteins and guide RNA combinations shown in TABLE 10 below were screened by an in vitro enrichment (IVE) assay to determine PAM recognition by each effector protein-guide RNA complex. Briefly, effector proteins were complexed with corresponding guide RNAs for 15 minutes at 37° C. The complexes were added to an IVE reaction mix. PAM screening reactions used 10 μl of RNP in 100 μl reactions with 1,000 ng of a plasmid library containing a randomized PAM sequence 3′ of a target protospacer (5′-NNNNNNN-3′, where N is any of A, C, G, T) in 1× Cutsmart buffer and were carried out for 15 minutes at 25° C., 45 minutes at 37° C., and 15 minutes at 45° C. Reactions were terminated with 1 μl of proteinase K and 5 μl of 500 mM EDTA for 30 minutes at 37° C. Any target plasmid that was successfully cleaved had an adapter ligated to the cut end, enabling PCR amplification. An EcoRI site was included near the spacer and this was used as a positive control. Next generation sequencing was performed on cut sequences to identify enriched PAMs.

TABLE 10
Effector Protein Guide Complexes

| | Guide Nucleic Acid | |
|---|---|---|
| Effector Protein | Name (SEQ ID NO for | Consensus |
| Name (SEQ ID NO) | DNA/RNA) | 3' PAM sequence |
| 2178307 (SEQ ID NO: 1) | R7793 (SEQ ID NO: 228/327) | NNNAAT (SEQ ID NO: 79) |
| 2178512 (SEQ ID NO: 2) | R7776 (SEQ ID NO: 229/ | NNGNMA (SEQ ID NO: 80) |
| | SEQ ID NO: 328) | |
| 2178891 (SEQ ID NO: 5) | R7821 (SEQ ID NO: 233/332) | RNGKNK (SEQ ID NO: 81) |
| 2179018 (SEQ ID NO: 9) | R7795 (SEQ ID NO: 237/336) | NNNAWWT (SEQ ID NO: 82) |
| 2179042 (SEQ ID NO: 10) | R7822 (SEQ ID NO: 238/337) | NNG (SEQ ID NO: 83) |
| 2179044 (SEQ ID NO: 11) | R7833 (SEQ ID NO: 240/339) | NNGRM (SEQ ID NO: 84) |
| 2179107 (SEQ ID NO: 14) | R7825 (SEQ ID NO: 243/342) | WNRRAW (SEQ ID NO: 85) |
| 2179109 (SEQ ID NO: 15) | R7826 (SEQ ID NO: 244/343) | NNGRA (SEQ ID NO: 86) |
| 2180100 (SEQ ID NO: 16) | R7784 (SEQ ID NO: 245/344) | HNYAR (SEQ ID NO: 87) |
| 2180100 (SEQ ID NO: 16) | R7785 (SEQ ID NO: 246/345) | HNYAR (SEQ ID NO: 87) |
| 2180475 (SEQ ID NO: 17) | R7837 (SEQ ID NO: 247/346) | NNGKT (SEQ ID NO: 88) |
| 2180475 (SEQ ID NO: 17) | R7847 (SEQ ID NO: 248/347) | NGG (SEQ ID NO: 89) |
| 2181249 (SEQ ID NO: 20) | R7838 (SEQ ID NO: 252/351) | NGG (SEQ ID NO: 89) |
| 2181249 (SEQ ID NO: 20) | R7848 (SEQ ID NO: 253/352) | NGGR (SEQ ID NO: 90) |
| 2181365 (SEQ ID NO: 21) | R7828 (SEQ ID NO: 254/353) | NGGNR (SEQ ID NO: 91) |
| 2181445 (SEQ ID NO: 22) | R7805 (SEQ ID NO: 255/354) | NNNMAWT (SEQ ID NO: 92) |
| 2181509 (SEQ ID NO: 23) | R7794 (SEQ ID NO: 256/355) | NRNACYN (SEQ ID NO: 93) |
| 2182747 (SEQ ID NO: 29) | R7771 (SEQ ID NO: 265/364) | NNNAHAA (SEQ ID NO: 94) |
| 2183116 (SEQ ID NO: 31) | R7813 (SEQ ID NO: 267/366) | NNNWWWT (SEQ ID NO: 95) |
| 2183137 (SEQ ID NO: 32) | R7815 (SEQ ID NO: 268/367) | NRNAAY (SEQ ID NO: 96) |
| 2183156 (SEQ ID NO: 33) | R7819 (SEQ ID NO: 269/368) | NNRACY (SEQ ID NO: 97) |
| 2183168 (SEQ ID NO: 34) | R7773 (SEQ ID NO: 270/369) | NNNGMWM (SEQ ID NO: 98) |
| 2183262 (SEQ ID NO: 36) | R7811 (SEQ ID NO: 272/371) | NRNAWC (SEQ ID NO: 99) |
| 2183288 (SEQ ID NO: 37) | R7814 (SEQ ID NO: 273/372) | NNNMAWT (SEQ ID NO: 92) |
| 2183304 (SEQ ID NO: 38) | R7797 (SEQ ID NO: 274/373) | NNGNNNN (SEQ ID NO: 100) |
| 2183304 (SEQ ID NO: 38) | R7798 (SEQ ID NO: 275/374) | NNAARY (SEQ ID NO: 101) |
| 2183526 (SEQ ID NO: 40) | R7802 (SEQ ID NO: 277/376) | NNNMAWT (SEQ ID NO: 92) |
| 2183526 (SEQ ID NO: 40) | R7803 (SEQ ID NO: 278/377) | NNNMAWT (SEQ ID NO: 92) |
| 2183548 (SEQ ID NO: 41) | R7801 (SEQ ID NO: 279/378) | NNNMAWT (SEQ ID NO: 92) |
| 2183638 (SEQ ID NO: 43) | R7812 (SEQ ID NO: 282/381) | NNNAAWT (SEQ ID NO: 102) |
| 2183868 (SEQ ID NO: 44) | R7809 (SEQ ID NO: 283/382) | NNNMAWT (SEQ ID NO: 92) |
| 2184029 (SEQ ID NO: 45) | R7807 (SEQ ID NO: 284/383) | NNNMAWT (SEQ ID NO: 92) |
| 2184261 (SEQ ID NO: 46) | R7778 (SEQ ID NO: 285/384) | HNRNWW (SEQ ID NO: 103) |
| 2184558 (SEQ ID NO: 47) | R7786 (SEQ ID NO: 286/385) | HNYAAR (SEQ ID NO: 104) |
| 2184558 (SEQ ID NO: 47) | R7787 (SEQ ID NO: 287/386) | HNYAAR (SEQ ID NO: 104) |
| 2185070 (SEQ ID NO: 49) | R7840 (SEQ ID NO: 289/388) | NGGWY (SEQ ID NO: 105) |
| 2185440 (SEQ ID NO: 51) | R7769 (SEQ ID NO: 293/392) | WNAWMWW (SEQ ID NO: 106) |
| 2185499 (SEQ ID NO: 53) | R7841 (SEQ ID NO: 295/394) | WNGR (SEQ ID NO: 107) |
| 2185499 (SEQ ID NO: 53) | R7851 (SEQ ID NO: 296/395) | NNG (SEQ ID NO: 83) |
| 2185882 (SEQ ID NO: 54) | R7816 (SEQ ID NO: 297/396) | NGG (SEQ ID NO: 83) |
| 2185938 (SEQ ID NO: 55) | R7829 (SEQ ID NO: 298/397) | NNRAAA (SEQ ID NO: 108) |
| 2185986 (SEQ ID NO: 56) | R7768 (SEQ ID NO: 299/398) | WNWRRM (SEQ ID NO: 109) |
| 2186029 (SEQ ID NO: 57) | R7792 (SEQ ID NO: 300/399) | NNYGMNY (SEQ ID NO: 110) |
| 2186035 (SEQ ID NO: 58) | R7852 (SEQ ID NO: 302/401) | NGG (SEQ ID NO: 89) |
| 2186149 (SEQ ID NO: 59) | R7843 (SEQ ID NO: 303/402) | WRG (SEQ ID NO: 111) |
| 2186149 (SEQ ID NO: 59) | R7853 (SEQ ID NO: 304/403) | WRG (SEQ ID NO: 111) |
| 2186167 (SEQ ID NO: 60) | R7767 (SEQ ID NO: 305/404) | NNNGHWW (SEQ ID NO: 112) |
| 2186215 (SEQ ID NO: 62) | R7810 (SEQ ID NO: 307/406) | NAAAAW (SEQ ID NO: 113) |
| 2186264 (SEQ ID NO: 63) | R7830 (SEQ ID NO: 308/407) | WGGNR (SEQ ID NO: 114) |
| 2186416 (SEQ ID NO: 65) | R7791 (SEQ ID NO: 310/409) | HNNAA (SEQ ID NO: 115) |
| 2186474 (SEQ ID NO: 66) | R7844 (SEQ ID NO: 311/410) | WNGRW (SEQ ID NO: 116) |
| 2186573 (SEQ ID NO: 68) | R7789 (SEQ ID NO: 315/414) | NNNRANK (SEQ ID NO: 117) |
| 2186752 (SEQ ID NO: 71) | R7764 (SEQ ID NO: 318/417) | WNWWHWW (SEQ ID NO: 118) |
| 2186752 (SEQ ID NO: 71) | R7765 (SEQ ID NO: 319/418) | WNWWHWW (SEQ ID NO: 118) |
| 2186873 (SEQ ID NO: 73) | R7817 (SEQ ID NO: 321/420) | NGG (SEQ ID NO: 89) |
| 2187023 (SEQ ID NO: 75) | R7845 (SEQ ID NO: 323/422) | NGG (SEQ ID NO: 89) |
| 2187023 (SEQ ID NO: 75) | R7855 (SEQ ID NO: 324/423) | NGG (SEQ ID NO: 89) |
| 2187103 (SEQ ID NO: 76) | R7806 (SEQ ID NO: 325/424) | NNNMAWT (SEQ ID NO: 92) |

### Example 9. Effector Proteins Edit Genomic DNA in HEK293T Cells

Effector proteins were tested for their ability to produce indels in HEK293T cells. Briefly, a pair of a guide RNA and a plasmid encoding the effector proteins, as recited in TABLE 11, were delivered to the HEK293T cells by lipofection. The guide RNAs were engineered to target different loci adjacent to biochemically determined PAM sequences. Indels in the loci were detected by next generation sequencing of PCR amplicons at the targeted loci and indel percentage was calculated as the fraction of sequencing reads containing insertions or deletions relative to an unedited reference sequence. TABLE 11 summarizes % indel generated by each pair of the guide RNA and the plasmid encoding the effector proteins in the HEK293T cells. “No plasmid” and Cas9 were included as negative and positive controls, respectively.

TABLE 11
Effector Protein Guide Complexes

| Effector | | |
|---|---|---|
| Protein | | |
| Name | | |
| (SEQ | Guide Nucleic Acid | Indel |
| ID NO) | (SEQ ID NO for RNA) | % |
| 2179018 | UGGAUGAAACCCAGACACAUGUUUGAGAGUCUUGUUGAAAAACAA | + |
| (SEQ ID | GACGAGUGCAAAUAAGGUUUAACCGUAAUCGUCGAAAGACUCUGC | |
| NO: 9) | AUGGUGCAGAAAGAAAGAGGCUGUAUAAAGCCU (SEQ ID NO: 436) | |
| | AGAUACAUCUCAUAAAUAGUGUUUGAGAGCCCUGUAGAAAUACAG | +++ |
| | GGUGAGUUCAAAUAAGGCUUUAGCCGAAAUCGCACCUGAGUGUCC | |
| | GCAUUGUGCGGGAUUAAGAGAAUCGAAAGAUUC (SEQ ID NO: 437) | |
| | AUUCCUCCUCAGAUGCCACCGUUUGAGAGCCCUGUAGAAAUACAG | ++ |
| | GGUGAGUUCAAAUAAGGCUUUAGCCGAAAUCGCACCUGAGUGUCC | |
| | GCAUUGUGCGGGAUUAAGAGAAUCGAAAGAUUC (SEQ ID NO: 438) | |
| 2183868 | GGGAUCAAGCCGCUGCCACCGUUUGAGAGCCCUGUAGAAAUACAG | +++ |
| (SEQ ID | GGUGAGUUCAAAUAAGGCUUUAGCCGAAAUCGCACCUGAGUGUCC | |
| NO: 44) | GCAUUGUGCGGGAUUAAGAGAAUCGAAAGAUUC (SEQ ID NO: 439) | |
| | UGGAUGAAACCCAGACACAUGUUUGAGAGCCCUGUAGAAAUACAG | +++ |
| | GGUGAGUUCAAAUAAGGCUUUAGCCGAAAUCGCACCUGAGUGUCC | |
| | GCAUUGUGCGGGAUUAAGAGAAUCGAAAGAUUC (SEQ ID NO: 440) | |
| | CCCAGACACAUAGCAAUUCAGUUUGAGAGCCCUGUAGAAAUACAG | + |
| | GGUGAGUUCAAAUAAGGCUUUAGCCGAAAUCGCACCUGAGUGUCC | |
| | GCAUUGUGCGGGAUUAAGAGAAUCGAAAGAUUC (SEQ ID NO: 441) | |
| | AGAAUGGAAAGUCAAAUUUCGUUUGAGAGCCCUGUAGAAAUACAG | + |
| | GGUGAGUUCAAAUAAGGCUUUAGCCGAAAUCGCACCUGAGUGUCC | |
| | GCAUUGUGCGGGAUUAAGAGAAUCGAAAGAUUC (SEQ ID NO: 442) | |
| | AUCCAGCAGAGAAUGGAAAGGUUUGAGAGCCCUGUAGAAAUACAG | +++ |
| | GGUGAGUUCAAAUAAGGCUUUAGCCGAAAUCGCACCUGAGUGUCC | |
| | GCAUUGUGCGGGAUUAAGAGAAUCGAAAGAUUC (SEQ ID NO: 443) | |
| | CCACCAAAAGGCAAAGGAAUGUUUGAGAGCCCUGUAGAAAUACAG | +++ |
| | GGUGAGUUCAAAUAAGGCUUUAGCCGAAAUCGCACCUGAGUGUCC | |
| | GCAUUGUGCGGGAUUAAGAGAAUCGAAAGAUUC (SEQ ID NO: 444) | |
| 2183262 | ACCUUCUCCUUUGGGUCCAGGUUUGAGAACCCUGUAGAAAUACAG | ++ |
| (SEQ ID | GGUGAGUUCAAAUAAGGCAUUAGCCGAAAUUGCACCUGAUGUCCG | |
| NO: 36) | CAUUGUGCGGGAUUAAAGAGGAUCGAAGGAUUC (SEQ ID NO: 445) | |
| | GGGAAAGCCAGGGGCUCCAGGUUUGAGAACCCUGUAGAAAUACAG | ++ |
| | GGUGAGUUCAAAUAAGGCAUUAGCCGAAAUUGCACCUGAUGUCCG | |
| | CAUUGUGCGGGAUUAAAGAGGAUCGAAGGAUUC (SEQ ID NO: 446) | |
| | GGCAGAAGCCAUCUGCAAGGGUUUGAGAACCCUGUAGAAAUACAG | ++ |
| | GGUGAGUUCAAAUAAGGCAUUAGCCGAAAUUGCACCUGAUGUCCG | |
| | CAUUGUGCGGGAUUAAAGAGGAUCGAAGGAUUC (SEQ ID NO: 447) | |
| | CAUGGUAAACGGGGCCAUUAGUUUGAGAACCCUGUAGAAAUACAG | + |
| | GGUGAGUUCAAAUAAGGCAUUAGCCGAAAUUGCACCUGAUGUCCG | |
| | CAUUGUGCGGGAUUAAAGAGGAUCGAAGGAUUC (SEQ ID NO: 448) | |
| | UGUAUAUCCCAGGUUUCCCCGUUUGAGAACCCUGUAGAAAUACAG | +++ |
| | GGUGAGUUCAAAUAAGGCAUUAGCCGAAAUUGCACCUGAUGUCCG | |
| | CAUUGUGCGGGAUUAAAGAGGAUCGAAGGAUUC (SEQ ID NO: 449) | |
| 2186167 | CCCUGGCCCCACCAAGUGGGGUUUAAGUACCUGGUAGGAAAUUAU | + |
| (SEQ ID | CAGAUCUACUUAAACAAGGCUUUAUGCCGAAAUCAAGGACAUCGA | |
| NO: 60) | CGGAUGUCC (SEQ ID NO: 450) | |
| | GGCCCCACCAAGUGGGACAAGUUUAAGUACCUGGUAGGAAAUUAU | ++ |
| | CAGAUCUACUUAAACAAGGCUUUAUGCCGAAAUCAAGGACAUCGA | |
| | CGGAUGUCC (SEQ ID NO: 451) | |
| | UGAGGGCAAAGCCUUGCUGAGUUUAAGUACCUGGUAGGAAAUUAU | + |
| | CAGAUCUACUUAAACAAGGCUUUAUGCCGAAAUCAAGGACAUCGA | |
| | CGGAUGUCC (SEQ ID NO: 452) | |
| 2185986 | GGCCCCACCAAGUGGGACAAAUUUAAGUACCUGUAAGGAAAUUUA | ++ |
| (SEQ ID | CAGAUCUACUUAAAUAAGGCUUUAUGCCGAGAUUAAGGAGUGCCG | |
| NO: 56) | ACGGGUACUCC (SEQ ID NO: 453) | |
| | GAUAGAAAUUGGAUGUGAGGAUUUAAGUACCUGUAAGGAAAUUUA | +++ |
| | CAGAUCUACUUAAAUAAGGCUUUAUGCCGAGAUUAAGGAGUGCCG | |
| | ACGGGUACUCC (SEQ ID NO: 454) | |
| | UUUACAACAUAGCCACGAGGAUUUAAGUACCUGUAAGGAAAUUUA | + |
| | CAGAUCUACUUAAAUAAGGCUUUAUGCCGAGAUUAAGGAGUGCCG | |
| | ACGGGUACUCC (SEQ ID NO: 455) | |
| | UGAGGGCAAAGCCUUGCUGAAUUUAAGUACCUGUAAGGAAAUUUA | ++ |
| | CAGAUCUACUUAAAUAAGGCUUUAUGCCGAGAUUAAGGAGUGCCG | |
| | ACGGGUACUCC (SEQ ID NO: 456) | |
| | UCCUUCACAGGCAGAUAUUAAUUUAAGUACCUGUAAGGAAAUUUA | ++ |
| | CAGAUCUACUUAAAUAAGGCUUUAUGCCGAGAUUAAGGAGUGCCG | |
| | ACGGGUACUCC (SEQ ID NO: 457) | |
| 2182747 | CAAGUGGGGAAUGGCAAGCAAUUUAAGUACCUAGUAGGAAAUUAU | ++ |
| (SEQ ID | UAGAUCUACUUAAACAAGGCUUUAUGCCGAAAUCAAGGACACCGA | |
| NO: 29) | CGGGUGUCC (SEQ ID NO: 458) | |
| | UGGGGAAGAAGGUGUCUUCUAUUUAAGUACCUAGUAGGAAAUUAU | + |
| | UAGAUCUACUUAAACAAGGCUUUAUGCCGAAAUCAAGGACACCGA | |
| | CGGGUGUCC (SEQ ID NO: 459) | |
| | UGAUAGCAUUAGCUGGCCGCAUUUAAGUACCUAGUAGGAAAUUAU | ++ |
| | UAGAUCUACUUAAACAAGGCUUUAUGCCGAAAUCAAGGACACCGA | |
| | CGGGUGUCC (SEQ ID NO: 460) | |
| | AGCCACAGCUCUGUCUCUGAAUUUAAGUACCUAGUAGGAAAUUAU | + |
| | UAGAUCUACUUAAACAAGGCUUUAUGCCGAAAUCAAGGACACCGA | |
| | CGGGUGUCC (SEQ ID NO: 461) | |
| | UAACCUAAAAGGAGCCUACCAUUUAAGUACCUAGUAGGAAAUUAU | + |
| | UAGAUCUACUUAAACAAGGCUUUAUGCCGAAAUCAAGGACACCGA | |
| | CGGGUGUCC (SEQ ID NO: 462) | |
| 2184261 | CGGGAACCAGAUCUCUCACCAUUUAAGUACCCGGUGAGAAAUUAU | + |
| (SEQ ID | CUUCGGACCUAUUUAAACAAGGCUUUAUGCCGAAAG (SEQ ID NO: | |
| NO: 46) | 463) | |
| | CGAUUCAAGUGGGGAAUGGCAUUUAAGUACCCGGUGAGAAAUUAU | + |
| | CUUCGGACCUAUUUAAACAAGGCUUUAUGCCGAAAG (SEQ ID NO: | |
| | 464) | |
| | UUUUGGAAACCAGCAGAAAGAUUUAAGUACCCGGUGAGAAAUUAU | + |
| | CUUCGGACCUAUUUAAACAAGGCUUUAUGCCGAAAG (SEQ ID NO: | |
| | 465) | |
| 2186573 | GGCACCAAUGUAGAUACCAAGUUUAGUUACCAUAAAGGAAAUUUU | + |
| (SEQ ID | AUGACCUAACUAAACAAGGGUUUAUCCCGGAUUCGGCUCUUCGGA | |
| NO: 68) | GCC (SEQ ID NO: 466) | |
| 2179109 | AGGGAAGGACCAGGGGAUGGGUUUAAGAUCUAUGUCAGAAAUGAC | + |
| (SEQ ID | AUAGAGAGUUUAAAUAAAGGUUUUACCCUAAAUACUCUUCUGAGU | |
| NO: 15) | UGCUGCUUAGGCA (SEQ ID NO: 467) | |
| | GAGGCGGUGAGCCGGGCAGGGUUUAAGAUCUAUGUCAGAAAUGAC | + |
| | AUAGAGAGUUUAAAUAAAGGUUUUACCCUAAAUACUCUUCUGAGU | |
| | UGCUGCUUAGGCA (SEQ ID NO: 468) | |
| | GGAGAAGAGGGGCAAUGCAGGUUUAAGAUCUAUGUCAGAAAUGAC | +++ |
| | AUAGAGAGUUUAAAUAAAGGUUUUACCCUAAAUACUCUUCUGAGU | |
| | UGCUGCUUAGGCA (SEQ ID NO: 469) | |
| | GUCUUGCUGCUCCUCCCGGCGUUUAAGAUCUAUGUCAGAAAUGAC | + |
| | AUAGAGAGUUUAAAUAAAGGUUUUACCCUAAAUACUCUUCUGAGU | |
| | UGCUGCUUAGGCA (SEQ ID NO: 470) | |
| | CCACCUGGCUUCCCAAUGCCGUUUAAGAUCUAUGUCAGAAAUGAC | + |
| | AUAGAGAGUUUAAAUAAAGGUUUUACCCUAAAUACUCUUCUGAGU | |
| | UGCUGCUUAGGCA (SEQ ID NO: 471) | |
| | CCUGGAGGGCCUGGGGGUCCGUUUAAGAUCUAUGUCAGAAAUGAC | + |
| | AUAGAGAGUUUAAAUAAAGGUUUUACCCUAAAUACUCUUCUGAGU | |
| | UGCUGCUUAGGCA (SEQ ID NO: 472) | |
| | GCCCAACUAAUAUAAACACCGUUUAAGAUCUAUGUCAGAAAUGAC | + |
| | AUAGAGAGUUUAAAUAAAGGUUUUACCCUAAAUACUCUUCUGAGU | |
| | UGCUGCUUAGGCA (SEQ ID NO: 473) | |
| | ACCGGCGCCAAGAAUGGGCAGUUUAAGAUCUAUGUCAGAAAUGAC | ++ |
| | AUAGAGAGUUUAAAUAAAGGUUUUACCCUAAAUACUCUUCUGAGU | |
| | UGCUGCUUAGGCA (SEQ ID NO: 474) | |
| | AGCUGUUUUGAAGACUCUCCGUUUAAGAUCUAUGUCAGAAAUGAC | + |
| | AUAGAGAGUUUAAAUAAAGGUUUUACCCUAAAUACUCUUCUGAGU | |
| | UGCUGCUUAGGCA (SEQ ID NO: 475) | |
| | GAGGUGCAGUAAUUCAGGCCGUUUAAGAUCUAUGUCAGAAAUGAC | ++ |
| | AUAGAGAGUUUAAAUAAAGGUUUUACCCUAAAUACUCUUCUGAGU | |
| | UGCUGCUUAGGCA (SEQ ID NO: 476) | |
| 2181249 | CAGUUUGGCAGGCGACCAGUGUUUUAGAAUUAUGUUAGAAAUAGC | +++ |
| (SEQ ID | AUAAUGAGUUAAAAUAAGGUUAAACCUUUAAUGCUGAUUUAUAUC | |
| NO: 20) | AGUUUCGCUUAGG (SEQ ID NO: 477) | |
| 2185882 | AGAUACAUCUCAUAAAUAGUGUUUGAGAACCUUGUAGAAAUACAA | + |
| (SEQ ID | GGUGAGUGCAAAUAAGGAUUUAUCCGACAUCGCCGUUUGGUCUGC | |
| NO: 54) | AUGGUGCAGGUAAAAGACUUGGUUUCAAGUCUU (SEQ ID NO: 478) | |
| | AUUCCUCCUCAGAUGCCACCGUUUGAGAACCUUGUAGAAAUACAA | ++ |
| | GGUGAGUGCAAAUAAGGAUUUAUCCGACAUCGCCGUUUGGUCUGC | |
| | AUGGUGCAGGUAAAAGACUUGGUUUCAAGUCUU (SEQ ID NO: 479) | |
| | GGGAUCAAGCCGCUGCCACCGUUUGAGAACCUUGUAGAAAUACAA | +++ |
| | GGUGAGUGCAAAUAAGGAUUUAUCCGACAUCGCCGUUUGGUCUGC | |
| | AUGGUGCAGGUAAAAGACUUGGUUUCAAGUCUU (SEQ ID NO: 480) | |
| | UGGAUGAAACCCAGACACAUGUUUGAGAACCUUGUAGAAAUACAA | +++ |
| | GGUGAGUGCAAAUAAGGAUUUAUCCGACAUCGCCGUUUGGUCUGC | |
| | AUGGUGCAGGUAAAAGACUUGGUUUCAAGUCUU (SEQ ID NO: 481) | |
| | CCCAGACACAUAGCAAUUCAGUUUGAGAACCUUGUAGAAAUACAA | +++ |
| | GGUGAGUGCAAAUAAGGAUUUAUCCGACAUCGCCGUUUGGUCUGC | |
| | AUGGUGCAGGUAAAAGACUUGGUUUCAAGUCUU (SEQ ID NO: 482) | |
| | GACUUACUGAAGAAUGGAGAGUUUGAGAACCUUGUAGAAAUACAA | +++ |
| | GGUGAGUGCAAAUAAGGAUUUAUCCGACAUCGCCGUUUGGUCUGC | |
| | AUGGUGCAGGUAAAAGACUUGGUUUCAAGUCUU (SEQ ID NO: 483) | |
| | AUCCAGCAGAGAAUGGAAAGGUUUGAGAACCUUGUAGAAAUACAA | +++ |
| | GGUGAGUGCAAAUAAGGAUUUAUCCGACAUCGCCGUUUGGUCUGC | |
| | AUGGUGCAGGUAAAAGACUUGGUUUCAAGUCUU (SEQ ID NO: 484) | |
| 2178932 | CCCCAAAUUCUAAGCAGAGUAUUUAAGUACCUGUAAGGAAAUUUA | +++ |
| (SEQ ID | CAGAUCUACUUAAACAAGGCUUUAUGCCGAAAUCAAGGACAUCGA | |
| NO: 6) | CGGAUGUCC (SEQ ID NO: 485) | |
| | CCCUACAUUUUGUGCAUAAAAUUUAAGUACCUGUAAGGAAAUUUA | + |
| | CAGAUCUACUUAAACAAGGCUUUAUGCCGAAAUCAAGGACAUCGA | |
| | CGGAUGUCC (SEQ ID NO: 486) | |
| | AAAUACAGAGGAAAUUAAGCAUUUAAGUACCUGUAAGGAAAUUUA | +++ |
| | CAGAUCUACUUAAACAAGGCUUUAUGCCGAAAUCAAGGACAUCGA | |
| | CGGAUGUCC (SEQ ID NO: 487) | |
| | CCUCCCUGGUCUCAGCCACCAUUUAAGUACCUGUAAGGAAAUUUA | +++ |
| | CAGAUCUACUUAAACAAGGCUUUAUGCCGAAAUCAAGGACAUCGA | |
| | CGGAUGUCC (SEQ ID NO: 488) | |
| | UCUCAAACAAAUGUGUCACAAUUUAAGUACCUGUAAGGAAAUUUA | +++ |
| | CAGAUCUACUUAAACAAGGCUUUAUGCCGAAAUCAAGGACAUCGA | |
| | CGGAUGUCC (SEQ ID NO: 489) | |
| | UUUGUACAAGAUCCGCAGACAUUUAAGUACCUGUAAGGAAAUUUA | +++ |
| | CAGAUCUACUUAAACAAGGCUUUAUGCCGAAAUCAAGGACAUCGA | |
| | CGGAUGUCC (SEQ ID NO: 490) | |
| | GGCAACUCAGAAGCAGGUGAAUUUAAGUACCUGUAAGGAAAUUUA | +++ |
| | CAGAUCUACUUAAACAAGGCUUUAUGCCGAAAUCAAGGACAUCGA | |
| | CGGAUGUCC (SEQ ID NO: 491) | |
| | AAGUAGGUUCAUCUUUCUCCAUUUAAGUACCUGUAAGGAAAUUUA | + |
| | CAGAUCUACUUAAACAAGGCUUUAUGCCGAAAUCAAGGACAUCGA | |
| | CGGAUGUCC (SEQ ID NO: 492) | |
| | CAUCUUGAAUCUGUUCCAUCAUUUAAGUACCUGUAAGGAAAUUUA | +++ |
| | CAGAUCUACUUAAACAAGGCUUUAUGCCGAAAUCAAGGACAUCGA | |
| | CGGAUGUCC (SEQ ID NO: 493) | |
| | UGCUCCCGGAGCCAAGGCUGAUUUAAGUACCUGUAAGGAAAUUUA | +++ |
| | CAGAUCUACUUAAACAAGGCUUUAUGCCGAAAUCAAGGACAUCGA | |
| | CGGAUGUCC (SEQ ID NO: 494) | |
| | UGGAGUAAGCGCCUGAGGUGAUUUAAGUACCUGUAAGGAAAUUUA | +++ |
| | CAGAUCUACUUAAACAAGGCUUUAUGCCGAAAUCAAGGACAUCGA | |
| | CGGAUGUCC (SEQ ID NO: 495) | |
| | CUUGGUGGAUACCCUGAAGUAUUUAAGUACCUGUAAGGAAAUUUA | ++ |
| | CAGAUCUACUUAAACAAGGCUUUAUGCCGAAAUCAAGGACAUCGA | |
| | CGGAUGUCC (SEQ ID NO: 496) | |

* Magnitude of Aligned Read percentage data: “+” represents a value under 5, “++” represents a value over 5 but under 10, “+++” represents a value over 10.

### Example 10. nD2L-932 and Reverse Transcriptase for Gene Editing in HEK293T Cells

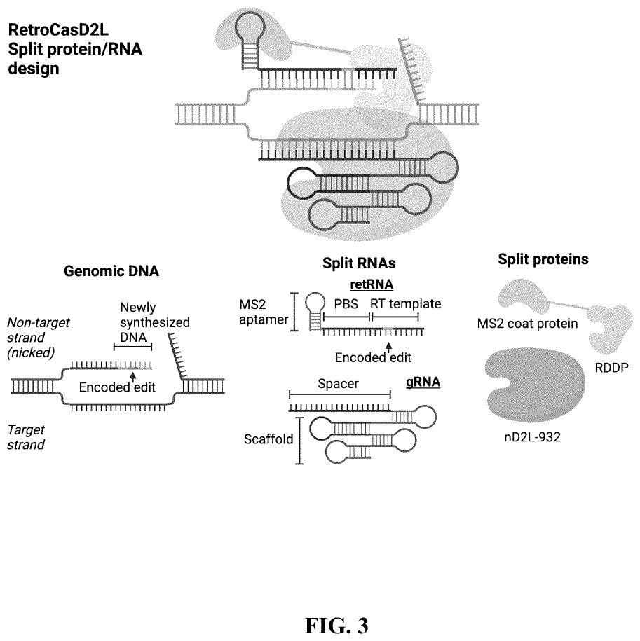

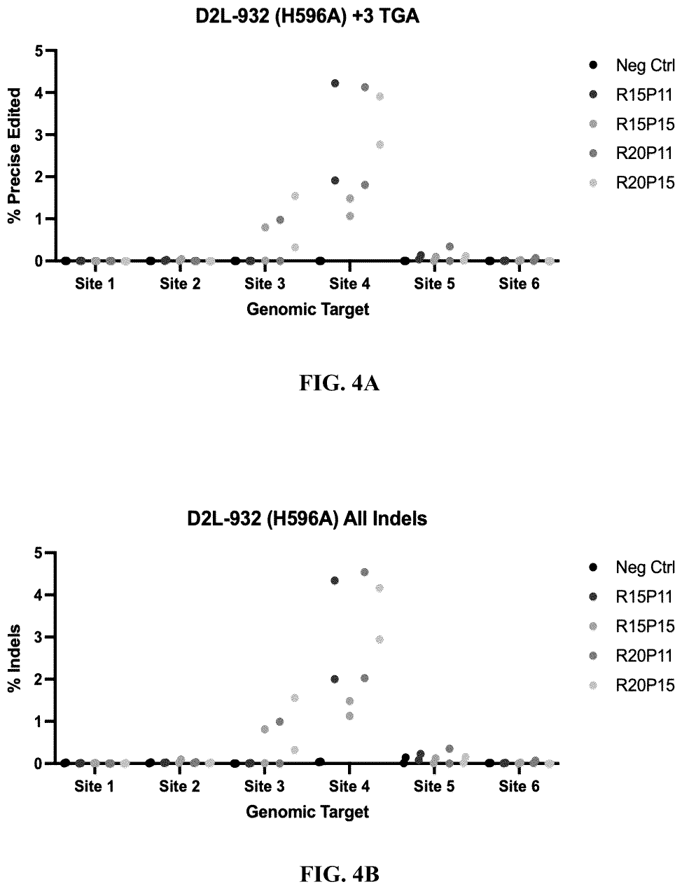

This Example demonstrates the ability of the effector protein 2178932-H596A (nD2L-932) to perform precise editing of a target nucleic acid. This experiment employed a split protein design and split RNA design, as shown in FIG. 3. Components of the system included: nD2L-932; a reverse transcriptase fused to a MS2 coat protein; one of several guide nucleic acids comprising a repeat sequence located 3′ of a 20 nt spacer sequence; and a template RNA (retRNA) comprising in order from 5′ to 3′ an MS2 aptamer, an RT template, and a primer binding sequence. The reverse transcriptase that was used included RNA-dependent DNA polymerase (RDDP) activity. The retRNA comprised an encoded edit of a 3 nucleotide insertion (5′-TGA-3′) relative to the target sequence. HEK293T cells were transiently transfected with these components. NGS sequencing revealed that the insertion was obtained with this system. Data are provided in FIGS. 4A-4B.

### Example 11. nD2L-932 and Reverse Transcriptase for Enhanced Gene Editing in HEK293T Cells

The ability of any one of the effector proteins of TABLE 1 to perform enhanced gene editing of a target nucleic acid is assayed as follows. Components of the system include: an effector protein; a reverse transcriptase fused to a MS2 coat protein; a first guide nucleic acid; a second guide nucleic acid; and a template RNA (retRNA) comprising in order from 5′ to 3′ an MS2 aptamer, an RT template, and a primer binding sequence. The reverse transcriptase that is used include RNA-dependent DNA polymerase (RDDP) activity. The two guide nucleic acids are designed to bind opposite strands, a target strand and a non-target strand, of the target nucleic acid, wherein the second site is downstream to the first site. HEK293T cells are transiently transfected with these components. This will generate nicking of the target strand at a first site and the non-target strand at a second site downstream of the first site. NGS sequencing is used for assessing gene editing of a target nucleic acid with this system. Without being bound by theory, such a system will advantageously enhance editing signal relative to a system that uses only one guide nucleic acid because in resolving whether to retain the edited or unedited strand of the target nucleic acid, a nick on the unedited strand signals to the cell's repair system that it's damaged and therefore leads to preferential removal.

What is claimed is:

1-340. (canceled)

341. A composition comprising:
(a) an effector protein, or a nucleic acid encoding the effector protein, wherein the effector protein comprises an amino acid sequence that is at least 95% identical to SEQ ID NO: 6; and
(b) a guide nucleic acid, or a nucleic acid encoding the guide nucleic acid, wherein the guide nucleic acid comprises a repeat sequence and a spacer sequence,
wherein the spacer sequence is complementary to a target sequence within a eukaryotic sequence.

342. The composition of claim 341, wherein the effector protein comprises an amino acid substitution of H596A relative to SEQ ID NO: 6.

343. The composition of claim 341, wherein the effector protein comprises an amino acid sequence of SEQ ID NO: 499.

344. The composition of claim 341, wherein the repeat sequence comprises a nucleotide sequence that is at least 90% identical to SEQ ID NO: 147.

345. The composition of claim 341, wherein the guide nucleic acid comprises a nucleotide sequence that is at least 90% identical to SEQ ID NO: 224.

346. The composition of claim 341, wherein the target sequence is adjacent to a protospacer adjacent motif of 5′-NNGTAW-3′ (SEQ ID NO: 119).

347. The composition of claim 341, wherein the repeat sequence is located 3′ of the spacer sequence.

348. The composition of claim 341, wherein the effector protein is linked to a fusion partner protein.

349. The composition of claim 348, wherein the effector protein exhibits nickase activity upon contacting with a double-stranded nucleic acid.

350. The composition of claim 348, wherein the fusion partner protein comprises a reverse transcriptase.

351. The composition of claim 350, wherein the reverse transcriptase is fused to the effector protein.

352. The composition of claim 351, wherein the composition comprises a template RNA.

353. The composition of claim 352, wherein the template RNA comprises an aptamer, and wherein the fusion partner protein further comprises an aptamer binding protein that binds the aptamer.

354. The composition of claim 341, comprising a lipid nanoparticle.

355. A pharmaceutical composition comprising the composition of claim 341, and a pharmaceutically acceptable excipient.

356. A cell comprising or modified by the composition of claim 341.

357. A method of modifying a target nucleic acid in a cell comprising contacting the cell with the composition of claim 341, thereby modifying the cell.

358. A method of treating a disease in a subject in need thereof, wherein the method comprises administering the composition of claim 341 to the subject.

359. The method of claim 358, wherein the disease is selected from a disease in TABLE 7.

360. The method of claim 358, wherein the disease is associated with a mutation in a gene selected from TABLE 8.
